(19) United States
(12) Patent Application Publication
(10) Pub. No.: US 2025/0325469 A1
(43) Pub. Date: Oct. 23, 2025

(54) Inhibitors of atp synthase - cosmetic and therapeutic uses

(71) Applicant: Michael David Forrest, Poole (GB)

(72) Inventor: Michael David Forrest, Poole (GB)

(21) Appl. No.: 18/271,488

(22) PCT Filed: Jan. 24, 2021
(86) PCT No.: PCT/IB2021/050529

§ 371 Date: Jul. 10, 2023

(51) Int. Cl. A61K 8/64; A61K 8/60; A61K 38/17; A61K 48/00; A61Q 19/08; C07K 14/47

(52) U.S. Cl. CPC A61K 8/64; A61K 8/606; A61K 38/17; A61K 48/005; A61Q 19/08; C07K 14/47; A61K 2800/86; C07K 2319/07; C07K 2319/10

(57) ABSTRACT

With supporting experimental data, this disclosure teaches that IF1 protein activity is a molecular determinant of lifespan, therein explaining why different species have different maximal lifespans, and it teaches a IF1 protein/fragment (or sequence variant thereof), or a fusion protein thereof, optionally a fusion protein comprising a Cell Penetrating Peptide (CPP) sequence, as an agent to slow/delay/reduce aging in a subject, optionally as a component of a cosmetic, optionally to treat an age-correlated disease/disorder. Moreover it teaches other inhibitors of F1F0 ATP hydrolysis, including small molecules, of a number of different scaffolds, for this purpose. Furthermore, with supporting experimental data, it teaches that compounds that slow the ATP-hydrolysing mode of ATP synthase are useful for treating various diseases and disorders, including cancer, particularly cancers that utilise the Warburg effect.

### RELATED APPLICATIONS

PCT/EP2018/051127 (published as WO2018/134265A1), PCT/EP2018/069175 (published as WO2019/012149A1), and their corresponding 371 national entry applications into the USA (U.S. application Ser. No. 16/478,497 [published as US2020/0247758A1] and U.S. application Ser. No. 16/629,390 [published as US2020/0306253A1] respectively), Canadian application number 3,050,553 and Australian application number AU2019208238 are all by the same inventor as the present application and are all herein incorporated in their entirety by reference (and the entire content of references [cited papers, patents and applications thereof] therein). Also incorporated in its entirety by reference is applicant's reply to the “written opinion of the International Search Authority” for PCT/EP2018/069175, which is publically available on the European Patent Register file for the EP entry of this PCT: EP application number 18746115.7 [published as EP3652156].

### FIELD OF THIS DISCLOSURE

This application discloses compounds that preferentially slow the ATP-hydrolysing mode of ATP synthase, pharmaceutical compositions of these compounds, and methods of use for slowing/delaying/reducing aging in a subject, which has both cosmetic and therapeutic applications, and treating subjects known to have various diseases or disorders, including cancer (e.g. diagnosed with), subjects suspected of having various diseases or disorders, including cancer, or subjects at risk of developing various diseases or disorders, including cancer. In particular embodiments, the subject is a human. In further embodiments, the subject is a companion/pet, or farm or laboratory animal.

### BACKGROUND OF THIS DISCLOSURE

### ATP Synthase

ATP synthase (also known as F₁F₀ ATP synthase, F₀F₁ ATP synthase, F₁F₀-ATPase, F₀F₁-ATPase, F₁F₀ ATP hydrolase) is located at the inner mitochondrial membrane (IM). It can use the proton motive force (pmf) to generate ATP from ADP and Pi [1-3]. ATP synthase is reversible and—depending on its substrate/product concentrations, the pmf and the voltage across inner mitochondrial membrane {Ψ_{IM}}—it can work “forwards” (passaging protons, making ATP) or “backwards” (pumping protons, consuming ATP): its “forward” and “reverse” modes respectively, which may also be termed F₁F₀ ATP synthesis and F₁F₀ ATP hydrolysis respectively.

### IF1 Protein

IF1 (or IF₁) is an endogenous protein, encoded by the ATPIF1 gene, which selectively blocks the reverse mode of ATP synthase [4]. Its activity is pH sensitive and low, but non-zero, at normal matrix pH, and significant upon matrix acidification, caused by collapse of the proton motive force across the mitochondrial inner membrane.

### SUMMARY OF THIS DISCLOSURE

A teaching of this disclosure is that reducing F₁F₀ ATP hydrolysis in a subject can slow/delay/reduce aging in that subject. Any anti-aging drug that targets/inhibits/reduces F₁F₀ ATP hydrolysis is componentry to this disclosure. This application discloses numerous anti-aging drug examples, many of which are also new compositions of matter, and discloses rationale and methods to find further examples, which are, in turn, encompassed by this disclosure and componentry to this application.

Compounds of this disclosure, which reduce F₁F₀ ATP hydrolysis in a subject, can be used to (a) slow/delay/reduce aging in a subject (which has cosmetic applications) and/or (b) treat/ameliorate/prevent/combat diseases, disorders and conditions, including age-correlated thereof (risk of incidence increases with age), and including cancer, wherein—without seeking restriction by theory—the abnormally glycolytic metabolism of cancers (Warburg effect), especially used by the most dangerous thereof (e.g. most refractory to present chemo/radio-therapies), hinges upon abnormally high rates of F₁F₀ ATP hydrolysis (consuming glycolytic ATP, releasing glycolysis from ATP negative feedback inhibition, yielding higher glycolytic rate, thence more glycolytic intermediates available to be shunted into biosynthesis, enabling faster proliferation, and more NADPH produced, enabling more Reactive Oxygen Species {ROS} mitigation, and less ROS produced because oxidative phosphorylation {OXPHOS} disfavoured by high proton motive force {pmf} across mitochondrial inner membrane created by F₁F₀ ATP hydrolysis, so [ROS] is lower, enabling enduring information fidelity of DNA and “limitless replicative potential” (i.e. immortality, which is distinctive trait of cancer, which normal cells don't share)), which a compound of this disclosure disrupts, conferring anti-cancer activity. Moreover compounds of this disclosure attack cancer characteristics shared with embryonic stem cells, which incidentally are immortal, and which aren't found in the adult human body, but are in the blastocyst ˜5 days after fertilization. Thus compounds of this disclosure have utility as emergency contraceptives, for preventing unwanted pregnancy, with a later time window than the “morning after pill”. In normal adult cells, reducing F₁F₀ ATP hydrolysis reduces a futile cycle of ATP synthesis and hydrolysis, used by the body for heat generation. If exogenous heat replaces this reduced endogenous heat (higher room temperature, wearing more clothes, geographical relocation to the tropics etc.), this reduces energy (food) consumption and treats/ameliorates/prevents/combats cachexia, cancer driven cachexia and/or weight loss, wherein cachexia is the biggest cause of death in cancer patients. Reducing this ATP synthesis/hydrolysis cycle means the oxidative phosphorylation rate is slower, less ROS are produced and the body accumulates less ROS damage per unit time i.e. aging slows. Therefore, F₁F₀ ATP hydrolysis inhibitors of this disclosure extend lifespan and healthspan, can treat/ameliorate/prevent/combat accelerated aging diseases, progeroid syndromes and the diseases of aging (e.g. Alzheimer's disease, dementia, Parkinson's disease, cancer etc.). It is noteworthy that compounds of this disclosure both treat cancer and slow aging, whereas many present cancer treatments accelerate aging, causing greater incidence of age related disease(s) and ailments. Also, it is noteworthy that compounds of this disclosure both treat and prevent cancer, whereas many present cancer treatments (e.g. radiotherapy) increase cancer risk. Activated macrophages are distinct from resting macrophages, and other normal adult cells, because the nitric oxide they produce to kill pathogens switches off their use of oxidative phosphorylation and they rely on F₁F₀ ATP hydrolysis to maintain Ψ_{IM}. Compounds of this disclosure inhibit F₁F₀ ATP hydrolysis and so depolarise Ψ_{IM} in activated (not resting) macrophages, which triggers their apoptosis. Compounds of this disclosure treat/ameliorate/prevent/combat macrophage associated diseases or disorders (e.g. Macrophage Activation Syndrome, HIV hides safely in activated macrophages during anti-retroviral therapy {ART} and from here repopulates HIV virus in blood plasma when ART is interrupted or discontinued, virus neuroinvasion via macrophages, thence HIV-associated neurocognitive disorders). F₁F₀ ATP hydrolysis inhibitors, by increasing metabolic/bioenergetic efficiency (less heat produced), can cause energy/weight gain in a subject, which has therapeutic, aesthetic, physical/mental performance applications, and commercial applications in livestock and farming. Compounds of this disclosure reduce F₁F₀ ATP hydrolysis and can reduce body temperature to a value controlled by intersection of compound dosage and ambient temperature (even at maximum possible effect, compound can't make body fall below, only to, ambient temperature; body temperature controlled by controlling ambient temperature), which can treat/ameliorate/prevent/combat a disease or disorder that drives to and/or causes a higher than normal body temperature (e.g. fever, infection, sepsis, malignant hyperthermia, neuroleptic malignant syndrome etc.) and a disease or disorder combated (or surgery or medical treatment helped) by hypothermia (e.g. neuroprotection/cardioprotection/tissue protection after a stroke or ischemia, deep hypothermic circulatory arrest for surgery etc.). Disclosed herein are the first drugs to treat/ameliorate/prevent/combat emergency grade hyperthermia, wherein hyperthermia is an extremely dangerous aspect to many Emergency Room (ER) admissions e.g. in some trauma patients. This is a valuable contribution to the art. Inhibiting F₁F₀ ATP hydrolysis reduces body temperature, which slows/reduces neural activity, wherein as regards body temperature, large reduction confers sedation, with applications to sleep and surgery etc., and smaller reduction confers anti-hyperactivity, anti-anxiety, anti-depression, anti-pain and treatment for premature ejaculation, epilepsy, Tourette's syndrome, Attention Deficit Hyperactivity Disorder (ADHD), Post Traumatic Stress Disorder (PTSD), homicidal/criminal/suicidal/self-harm ideation/tendency/thoughts etc. The intersection between F₁F₀ ATP hydrolysis inhibitor drug dose, and ambient temperature, dictates how much body temperature falls and thence depth of the sedation, wherein if ambient temperature equals 37° C., the drug can't reduce body temperature below this, no matter the dose, and no sedative action can occur. Drug action against a fundamental physiological parameter (body temperature), which dictates a further fundamental physiological parameter (action potential characteristic(s): firing threshold/conduction velocity/firing frequency etc.), yields incredibly broad therapeutic application. It combats any pathology/condition characterized by too much/inappropriate/undesired signals/activity/electrical activity in the nervous system. Juxtaposition of sedation with anti-aging action, which a compound of this disclosure confers, has applications to space travel, especially because the sedation can be turned on and off by settings of the ambient temperature.

By reducing metabolic heat production and body temperature a compound of this disclosure can treat all the many conditions in which the body gets too hot, can treat all the many conditions assisted by a lowered body temperature, which (because neuron firing characteristics are very temperature dependent) includes many neurological/mental disorders characterized by too much/unwanted neural activity, wherein the body temperature drop doesn't occur if the subject's ambient temperature and/or bodily insulation is sufficient to compensate for the lower metabolic heat production, wherein this lower metabolic rate slows the subject's aging, which can then slow/delay/prevent/treat the many diseases of aging (diseases/conditions with increased risk of occurrence with age), wherein the increased metabolic efficiency (less chemical energy of food dissipated as heat) can help treat cachexia, wasting and similar, wherein the compound adversely impacts the distinctive metabolic program of cancer, conferring anti-cancer activity, and selectively kills activated macrophages, and so it can treat the many diseases/disorders caused or exacerbated by activated macrophages, wherein many pathogens hide in safety from the immune system, and drug treatment, inside macrophages (which they inherently activate), for example HIV. When the compound is administered topically (e.g. to skin region), instead of systemically, the drop in metabolic heat production (and slower aging) is local, wherein the temperature of this region is maintained by heat transfer from other body areas, especially via blood flow, and so there is no temperature change, but there is slower aging in the administered region, which has cosmetic utility.

In some embodiments, a compound employed of this disclosure is an IF1 protein/fragment (or sequence variant thereof), or a fusion protein thereof, optionally a fusion protein comprising a Cell Penetrating Peptide (CPP) sequence, as an agent to slow/delay/reduce aging in a subject, optionally as a component of a cosmetic, optionally as a component of a therapeutic to treat at least one age-correlated disease/disorder.

### BRIEF DESCRIPTION OF THE DRAWINGS

FIG. 1 shows anti-cancer activity of compounds 8a and 8b in the NCI one-dose (10 μM) assay.

FIG. 2 compares the anti-cancer activities of compounds 6a, 6b, 7a, 7b, 8a and 8b in the NCI one-dose (10 μM) assay.

FIG. 3 consolidates anti-cancer activity data for 8a and 8b from the NCI one-dose and five-dose assay.

FIG. 4 presents experimental data showing that IF1 protein activity is a determinant of lifespan.

FIG. 5 relates to the in vivo effect of compound 6b.

FIG. 6 shows that, in vivo, inhibiting F₁F₀ ATP hydrolysis safely reduces the rate of oxidative phosphorylation and ROS generation, shown with forebrain neurons.

FIG. 7 shows that, in vivo, inhibiting F₁F₀ ATP hydrolysis safely reduces the rate of oxidative phosphorylation, shown with hepatocytes.

FIG. 8 shows that, in vivo, inhibiting F₁F₀ ATP hydrolysis safely reduces the rate of oxidative phosphorylation, shown in intestine (colonic cells).

FIG. 9 is a diagram illustrating how decreasing [ROS] in a cell, for example by inhibiting F₁F₀ ATP hydrolysis which reduces the oxidative phosphorylation/ROS generation rate, can prolong/increase the information fidelity of DNA, which slows/reverses aging.

FIG. 10 presents some peptide/protein sequence embodiments of this disclosure.

### DETAILED DESCRIPTION OF THE INVENTION

### Some Guidance and Definitions

All publications, patents and patent applications mentioned or cited in this disclosure (or the corresponding Application Data Sheet (ADS) and/or Information Disclosure Statement [IDS]) are herein incorporated, in entirety, by reference. This disclosure uses IC₅₀ and EC₅₀ interchangeably, for a process being inhibited or reduced. Chemical structures were drawn using the chemical drawing feature in [5], and if a drawing feature is unknown to the reader they are referred to its documentation, or to explore the software themselves: all clear to those of the art. Hydrogen on structures is typically not shown, present implicitly, but it is shown for some presented structures “On Hetero and Terminal” [5] groups. Herein, the symbol D is used for deuterium (²H). For compound synthesis schemes herein, starting materials are commercially available or can be readily prepared by one of ordinary skill in the art using known methods or derived by procedures analogous to those described in the literature. Examples and preparations herein describe the manner and process of making and using this disclosure. It should be understood that there will be other embodiments which fall within the spirit and scope of the disclosure. Where a term is provided in the singular, the inventor also contemplates the plural of that term. A phrase in the form “A/B” or in the form “A and/or B” means (A), (B), or (A and B). Herein, wherever “and” is used, in an alternative embodiment(s), “or” is used/substituted in its place. And wherever “or” is used, in an alternative embodiment(s) “and” is used/substituted in its place. Herein, when something is referred to in the singular (e.g. by prefixing with “a”/“an”/“the”), unless specifically stated otherwise, in alternative embodiments the plural form/plurality is also contemplated and componentry to this disclosure. As used herein with reference to the utilities described, the terms “treating” or “treatment” encompass both responsive and prophylaxis/preventative measures designed to inhibit/eradicate/prevent, reduce risk of and/or delay the onset/cause of the disease or disorder (or one or more of its symptoms), or to cure/eradicate, alleviate, abrogate, palliate, reverse, prevent, ameliorate, lessen, reduce, modulate, stabalize, delay, suppress, manage, reduce predisposition to, reduce risk of, prevent, reduce reoccurrence of, lengthen time to remission of, or slow progression/spread of the disease or disorder and/or one or more of its symptoms and/or increase quality/length of life and/or improve subject outcome/wellness. The terms “subject” and “patient” refer to organisms to be treated by the compounds/methods of the present disclosure and can refer to a human or animal. The terms “subject” and “patient” are used interchangeably herein, in reference, for example, to a mammalian subject, such as a human patient. The term “subject” refers to an animal, including, but not limited to, a primate (e.g. human, monkey, chimpanzee, gorilla, and the like), a rodent (e.g. rat, mouse, gerbil, hamster, ferret, and the like), a lagomorph, a swine (e.g. pig, miniature pig), an equine, a canine, a feline, and the like, a companion/exotic/farm/laboratory animal. As used herein, the term “therapeutically effective amount” or “effective amount” refers to the amount of a compound (e.g. a compound of the present disclosure) sufficient to effect a therapeutically/cosmetically/aesthetically beneficial/desired result including, for example, mitigating/alleviating to some extent (reducing frequency/duration/severity, and/or prevent development of) or eliminating one or more symptoms of the disease/disorder/condition/sub-optimum, or treating at least one physiological defect or pathology or etiology that causes or contributes to the disease/disorder/condition/sub-optimum being treated. In the case of aging and/or an aging correlated/driven disorder, an effective amount is that which slows the rate of aging, optionally which can slow the rate of one or more aging correlated/driven disorders. In the case of cancer, a therapeutically effective amount can be, for example that which slows/halts/stabalizes/regresses cancer proliferation/spread/invasion/malignancy/danger in the subject and/or which slows/halts/stabalizes/regresses cancer associated cachexia. A therapeutically effective amount accounts for treatment variables including, for example, dose, duration, timing and route of administration. Some disclosure embodiments are to administer a compound(s) of this disclosure to a subject diagnosed with cancer, suspected of having cancer, exhibiting symptoms of a cancer, at risk of cancer (e.g., a human who is genetically or otherwise predisposed to developing a cancer), susceptible to cancer, recovering/recovered from cancer or free of cancer. Palliative use of a compound(s) of this disclosure, optionally in a subject with cancer, is contemplated by, and componentry to, this disclosure. The term “therapeutically effective amount” or “effective amount” can also refer to the amount of compound that is sufficient to elicit the biological/medical/clinical response of a cell/tissue/system/animal/human that is being sought by a researcher/veterinarian/medical doctor/clinician. The term “therapeutically effective amount” or “effective amount” of a compound can also refer to a sufficient amount of the compound that provides a desired effect but with no, or acceptable, toxicity. This amount may vary from subject to subject, depending on the species, age, and physical condition of the subject, the severity of the disease that is being treated, the particular compound used, its mode of administration, and the like. A suitable “effective amount” may be determined by one of ordinary skill in the art. Further definition(s) of “therapeutically effective amount”/“effective amount” is found herein, in the disclosure section titled “Dosage”, which is also valid for use with this disclosure, wherein if this is no correspondence/overlap, or if there is a contradiction of definition(s), all definitions are valid but for different embodiments of the disclosure. This deconfliction, wherein conflicting definitions of a single word/phrase define different disclosure embodiments, is applicable to any conflicting/non-corresponding plurality of definition for a single word/phrase found herein.

In some embodiments, where the word “subject” is used in a sentence of this disclosure, it is substituted with “subject in need of treatment” or “subject in need thereof” or “subject in need/want thereof”. In some embodiments, where the word “effective” is used in a claim or statement in this disclosure, it is substituted with “therapeutically effective” or “cosmetically effective”. Three different claim types: method of medical treatment, Swiss-type and Product by process (purpose-limited-product format, EPC 2000); in this disclosure, when a claim or statement is given in one of these forms it also incorporates by reference the same subject matter in both the other claim forms.

The meaning of “concatenate” or “concatenated” at each point of use will be clear to one of the art given each context of its use. For example, when used with amino acid sequences it can refer to these sequences being covalently bound to one another (e.g. peptide bonded to one another). For example, when used with nucleotide sequences it can refer to these sequences being covalently bound to one another (e.g. by a phosphodiester linkage, or two thereof if the sequences are double stranded).

Cancer is herein used to mean any member of a class of diseases/disorders characterized by uncontrolled/undesirable/abnormal/dysregulated/unregulated, including harmful/dangerous (to health and/or lifespan), division of cells, including that independent of normal regulatory mechanisms (e.g. loss of contact inhibition). A “tumor” comprises one or more cancerous cells. Cancer cells, in some cases, gain the ability to invade other tissues, either by direct growth into adjacent tissue through invasion or by implantation into distant sites by metastasis. Metastasis is defined as the stage in which cancer cells are transported through the bloodstream or lymphatic system. The cancer may be, for illustrating example, a solid tumor, metastatic cancer, non-metastatic cancer, malignant cancer, benign cancer or pre-cancer. In some embodiments, the cancer may be a chemo-resistant or multidrug resistant cancer, i.e. a refractive form of cancer. It should be appreciated that a composition/compound of this disclosure may be used alone or in combination with one or more additional anti-cancer agents or treatments (e.g. chemotherapeutic agents, targeted therapeutic agents, pseudo-targeted therapeutic agents, hormones, radiation, surgery, etc., or any combination of two or more thereof), optionally a further composition(s)/compound(s) of this disclosure. In some embodiments, a composition(s)/compound(s) of this disclosure may be administered to a subject who has undergone a treatment involving one or more of surgery, radiation, chemotherapy. In certain embodiments, a composition or compound of this disclosure may be administered chronically to prevent, or reduce the risk of, a cancer recurrence. According to one embodiment, the subject to be treated is characterized by the presence of a precancerous condition, and the administering of the compound is effective to prevent development of the precancerous condition into the cancerous condition. This can occur by destroying the precancerous cell prior to or concurrent with its further development into a cancerous state. For the purpose of this disclosure, beneficial or desired results in the subject include, but are not limited to, alleviation of symptoms, diminishment of extent of disease, stabilized (i.e., not worsening) state of disease, delay or slowing of disease progression, amelioration or palliation of the disease state, remission (partial or total), disease prevention, or reducing predisposition to the disease, prolonging survival as compared to expected survival if not receiving treatment. In some embodiments, a compound(s) of the present disclosure is used to prevent the growth of a tumor or cancer, and/or prevent the metastasis of a tumor or cancer, and/or to shrink or destroy a cancer and/or treat complications of cancer. A treatment using one or more of the disclosed therapeutic compounds and compositions disclosed herein may decrease the growth rate of tumor cells, decrease the cell division rate of tumor cells, decrease the extent of invasion of tumor cells into adjacent tissue or organs, decrease the extent of metastasis, decrease angiogenesis, increase apoptosis, increase tumor cell death, increase tumor cell necrosis, or all or any combination thereof. A treatment using one or more of the disclosed therapeutic compounds and compositions disclosed herein may decrease hyperplasia, decrease the growth rate of hyperproliferating cells, decrease the cell division rate of hyperproliferating cells, decrease the extent to which hyperproliferating cells becomes cancerous, decrease angiogenesis, decrease nodule formation, decrease cyst formation, increase apoptosis, increase tumor cell death and/or increase tumor cell necrosis, or all or any combination thereof.

### Some Formalities

A pharmaceutical composition comprising at least one (optionally more than one) compound, as described herein, and a pharmaceutically-acceptable carrier or excipient or diluent. A pharmaceutical composition comprising at least one (optionally more than one) compound, as described herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and a pharmaceutically-acceptable carrier or excipient or diluent.

Use of a compound, specified herein, for treatment of a disease, specified herein. Use of a compound(s) defined herein, and/or use of a composition containing at least one compound defined herein, and/or use of a pharmaceutical composition defined herein, for treatment of one or more diseases or disorders specified herein. Use of a compound(s) defined herein, or a pharmaceutically-acceptable salt, solvate, hydrate or prodrug thereof, and/or use of a composition containing at least one compound defined herein, and/or use of a pharmaceutical composition defined herein, optionally a compound(s) and/or composition(s) that reduces F₁F₀ ATP hydrolysis in a subject, optionally at least one compound of at least one of Formula (I), (II), (III), (IV), (V), (VI), (VII), (VIII), [X], for treatment/amelioration/prevention/reversal/combat of one or more diseases or disorders or physiological processes (and/or one or more of its consequences) or an unwanted/undesirable aesthetic(s) specified herein.

Use of a compound(s) specified herein for the manufacture of a medicament. Use of a compound, specified herein, for manufacture of a medicament for treatment of a disease, specified herein. Use of a compound(s) defined herein, and/or use of a composition containing at least one compound defined herein, and/or use of a pharmaceutical composition defined herein, for the manufacture of a medicament for treatment of one or more diseases or disorders specified herein. Use of a compound(s) defined herein, or a pharmaceutically-acceptable salt, solvate, hydrate or prodrug thereof, and/or use of a composition containing at least one compound defined herein, and/or use of a pharmaceutical composition defined herein, optionally a compound(s) and/or composition(s) that reduces F₁F₀ ATP hydrolysis in a subject, optionally at least one compound of at least one of Formula (I), (II), (III), (IV), (V), (VI), (VII), (VIII), [X], for the manufacture of a medicament for treatment/amelioration/prevention/reversal/combat of one or more diseases or disorders or physiological processes (and/or one or more of its consequences) or an unwanted/undesirable aesthetic(s) specified herein.

A compound(s) and/or composition(s) described herein for use in a method of treatment of the human or animal body by therapy.

A compound(s) and/or composition(s) described herein for use in a method of treatment/improvement/enhancement of the human or animal body by therapy.

A method of treating, ameliorating, preventing or combating a disease or disorder by administering a therapeutically effective amount to the subject of at least one compound as defined herein. A method of treating, ameliorating, preventing or combating a disease or disorder by administering a therapeutically effective amount to the subject in need thereof of at least one compound defined as herein. A method of treating, ameliorating, preventing or combating a disease or disorder by administering to a subject in need of treatment an effective amount of at least one compound defined herein. A method of treating/ameliorating/preventing/reversing/combating one or more of a disease/disorder or physiological process (and/or one or more of its consequences) or an unwanted/undesirable aesthetic(s), specified herein, in a subject wherein the method comprises administering an effective amount to the subject of at least one compound defined herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and/or a pharmaceutical composition defined herein. A method of treating/ameliorating/preventing/reversing/combating one or more of a disease/disorder or physiological process (and/or one or more of its consequences) or an unwanted/undesirable aesthetic(s), specified herein, in a subject wherein the method comprises administering an effective amount to the subject in need/want thereof of at least one compound defined herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and/or a pharmaceutical composition defined herein.

Use of a compound(s) defined herein, or a pharmaceutically-acceptable salt, solvate, hydrate or prodrug thereof, and/or use of a composition containing at least one compound defined herein, and/or use of a pharmaceutical composition defined herein, optionally a compound(s) and/or composition(s) that reduces F₁F₀ ATP hydrolysis in a subject, optionally at least one compound of at least one of Formula (I), (II), (III), (IV), (V), (VI), (VII), (VIII), [X], for treatment/amelioration/prevention/reversal/combat of one or more diseases or disorders or physiological processes (and/or one or more of its consequences) or an unwanted/undesirable aesthetic(s) specified herein, in subject, wherein the administration to the subject is topical/local (not systemic). A method of treating/ameliorating/preventing/reversing/combating one or more of a disease/disorder or physiological process (and/or one or more of its consequences) or an unwanted/undesirable aesthetic(s), specified herein, in a subject, wherein the method comprises topically/locally (not systemically) administering an effective amount to the subject in need/want thereof of at least one compound defined herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and/or a pharmaceutical composition defined herein.

Use of a compound(s) defined herein, or a pharmaceutically-acceptable salt, solvate, hydrate or prodrug thereof, and/or use of a composition containing at least one compound defined herein, and/or use of a pharmaceutical composition defined herein, optionally a compound(s) and/or composition(s) that reduces F₁F₀ ATP hydrolysis in a subject, optionally at least one compound of at least one of Formula (I), (II), (III), (IV), (V), (VI), (VII), (VIII), [X], for treatment/amelioration/prevention/reversal/combat of one or more diseases or disorders or physiological processes (and/or one or more of its consequences) or an unwanted/undesirable aesthetic(s) specified herein, in a subject, wherein the subject is also administered with one or more compounds or compositions approved for human use, optionally for anti-cancer use, by the United States Food and Drug Administration (FDA) and/or European Medicines Agency (EMA), optionally in the same pharmaceutical composition. A method of treating/ameliorating/preventing/reversing/combating one or more of a disease/disorder or physiological process (and/or one or more of its consequences) or an unwanted/undesirable aesthetic(s), specified herein, in a subject, wherein the method comprises administering an effective amount to the subject in need/want thereof of at least one compound defined herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and/or a pharmaceutical composition defined herein, wherein the subject is also administered with an effective amount (which can be less than when administered alone) of one or more compounds or compositions approved for human use, optionally for anti-cancer use, by the United States Food and Drug Administration (FDA) and/or European Medicines Agency (EMA), optionally in the same pharmaceutical composition.

Use of a compound(s) defined herein, or a pharmaceutically-acceptable salt, solvate, hydrate or prodrug thereof, and/or use of a composition containing at least one compound defined herein, and/or use of a pharmaceutical composition defined herein, optionally a compound(s) and/or composition(s) that reduces F₁F₀ ATP hydrolysis in a subject, optionally at least one compound of at least one of Formula (I), (II), (III), (IV), (V), (VI), (VII), (VIII), [X], for treatment/amelioration/prevention/reversal/combat of one or more diseases or disorders or physiological processes (and/or one or more of its consequences) or an unwanted/undesirable aesthetic(s) specified herein, in a subject, wherein the mg/kg drug dose administered to the subject is comparable with or larger than the mg/kg dose administered to a subject of smaller bodily size (optionally a subject of another, smaller, species), which is very distinct from most drugs, and optionally the mg/kg dosage administered to adult humans is comparable or greater than the No Observed Adverse Effects Level (NOAEL) mg/kg dosage in mice housed at 22° C. A method of treating/ameliorating/preventing/reversing/combating one or more of a disease/disorder or physiological process (and/or one or more of its consequences) or an unwanted/undesirable aesthetic(s), specified herein, in a subject, wherein the method comprises administering an effective amount to the subject in need/want thereof of at least one compound defined herein, or a pharmaceutically-acceptable salt, solvate, hydrate or prodrug thereof, optionally at least one compound of at least one of Formula (I), (II), (III), (IV), (V), (VI), (VII), (VIII), [X], optionally a compound(s) that reduces F₁F₀ ATP hydrolysis in a subject, and/or a composition containing at least one compound defined herein, and/or a pharmaceutical composition defined herein, wherein the mg/kg drug dose administered to the subject is comparable with or larger than the mg/kg dose administered to a subject of smaller bodily size (optionally a subject of another, smaller, species), which is very distinct from most drugs, and optionally the mg/kg dosage administered to one or more adult humans is comparable or greater than the No Observed Adverse Effects Level (NOAEL) mg/kg dosage in mice housed at 22° C. This would greatly surprise someone of the art because it is very distinct from most other drugs, wherein bigger species are administered much lower mg/kg doses, not comparable or larger.

### Some Non-Limiting Aspects of the Teaching

In some of the most dangerous cancers, refractory to present [chemo/radio] therapies, during some or all of their cell cycle, Reactive Oxygen Species (ROS) decrease [NADPH], because NADPH is consumed in ROS mitigation processes, and this then pulls through increased pentose phosphate pathway (PPP) and glycolytic flux. But such a pivotal increase in glycolytic/PPP flux can only occur because of F₁F₀ ATP hydrolysis, a distinctive feature to these cancers, which stops ATP produced by glycolysis from accumulating and slowing glycolysis by negative feedback inhibition of key glycolytic enzymes. This increased PPP flux maintains [NADPH] and ROS mitigation. In this way, these cancers can maintain a very high ROS mitigation capability, maintain very low intracellular [ROS], and tend to be the most resistant to conventional [chemo/radio] therapies, which work, or often don't work (!), by increasing [ROS]. Compounds of this disclosure undermine this process/resistance. By inhibiting/reducing F₁F₀ ATP hydrolysis, they increase the anti-cancer efficacy of any chemical or treatment that increases Reactive Oxygen Species (ROS) in cancer cells. Some embodiments of this disclosure is any such co-treatment(s). Indeed, a compound(s) of this disclosure increases the success rate of standard of care [chemo/radio] therapies and optionally permits their use at lower dosing, which reduces their horrendous side-effects. This disclosure encompasses a compound(s) of this application, for example at least one compound of at least one of Formula (I), (II), (III), (IV), (V), (VI), (VII), (VIII), [X], in co-therapy/administration with one or more of surgery, chemotherapy, immunotherapy, immuno-oncology, radioimmunotherapy, biological therapy, hormone therapy, radiotherapy or any US Food and Drug Administration (FDA) and/or European Medicines Agency (EMA) approved drug(s) or treatment(s), for example, a drug/treatment approved for cancer therapy. In some embodiments the anti-cancer activity of a compound(s) of this disclosure, for example at least one compound of at least one of Formula (I), (II), (III), (IV), (V), (VI), (VII), (VIII), [X], adds to/synergizes with (potentiates) the anti-cancer activity of an FDA and/or EMA approved anti-cancer treatment(s) e.g. one or more of chemotherapy, radiotherapy, immunotherapy, surgery etc. In other words, their combined anti-cancer effect is greater than simply being the sum of each alone. In some embodiments, a compound(s) of this disclosure is used as an adjuvant or neoadjuvant to another cancer treatment(s) e.g. used as an adjuvant or neoadjuvant to chemo and/or radiotherapy and/or surgery. In some embodiments a compound(s) of this disclosure, for example at least one compound of at least one of Formula (I), (II), (III), (IV), (V), (VI), (VII), (VIII), [X], makes a cancer(s) more radiosensitive/less radioresistant and/or more chemosensitive/less chemoresistant i.e. more amenable to treatment by radio- and/or chemo-therapy, acts as a radiosensitizer and/or chemosensitizer. This is very valuable for treating radio- and/or chemo-resistant cancers. Chemotherapies are well known to those of the art, including, but not limited to, cisplatin, carboplatin, taxol, oxaliplatin etc, and tend to be (very) toxic. Encompassed herein is a method of reducing, treating and/or preventing adverse or undesired effects associated with conventional therapy including, but not limited to, chemotherapy, radiotherapy, immunotherapy, wherein a compound(s) provided herein, e.g. a compound(s) of one or more of Formula (I), (II), (III), (IV), (V), (VI), (VII), (VIII), [X], or a pharmaceutically acceptable salt, solvate, hydrate, prodrug thereof, is administered to a subject prior to, during, or after the occurrence of the adverse effect associated with conventional therapy, optionally wherein the dosage/frequency/use of the conventional therapy is decreased. In certain embodiments, a compound(s)/composition(s) disclosed herein can be administered to a subject in combination/co-therapy with one or more monoclonal antibodies such as one or more cancer immunotherapy monoclonal antibodies known in the art, including, but not limited to, at least one “checkpoint inhibitor” monoclonal antibody. In other embodiments, a compound(s) of this disclosure is used as cancer therapy alone.

### Body Temperature

Administered to a subject, a F₁F₀-ATP hydrolysis inhibitor(s) conserves ATP, so less ATP needs to be synthesized, therefore respiration rate slows, thence metabolic heat production declines and body temperature can fall towards ambient temperature (if ambient<body temperature). So, when the ambient temperature isn't arduous (not requiring significant energy consuming physiological/behavioural adaptations to maintain body temperature) and dietary intake stays constant, weight gain/maintenance can occur, which can assist cachexia, for example cancer driven cachexia. This is clinically valuable because cachexia is the leading cause of death in cancer patients. If the ambient temperature is sufficiently close to the required body temperature, then the aforementioned decrease in heat generation is safe, because the body temperature can't fall below the ambient temperature. So, for example, if the ambient temperature is 37° C., inhibiting F₁F₀-ATP hydrolysis could make body temperature fall to this ambient temperature, but not below it, and this is safe because ˜37° C. body temperature is safe. Inhibiting F₁F₀-ATP hydrolysis will reduce, but not abolish, metabolic heat production. So, body metabolism will still contribute to heating the body, just less so, which will shift the thermoneutral and thermal comfort zones (terms well known to those of the art [6], temperatures vary by species, as is well known to those of the art) to higher temperature(s). If the subject is located at a higher temperature to account for this shift, for example at their updated, higher thermoneutral temperature, or make behavioural adaptations (e.g. wearing more clothes), then this shift is harmless. An embodiment of this disclosure is setting the dosage of a compound(s) that inhibits F₁F₀ ATP hydrolysis with consideration of the ambient temperature, wherein higher dosages are permissible at higher ambient temperatures. The preferred ambient temperature for a dosage permits the subject to be thermoneutral, and/or thermal comfortable, without the metabolic heat (respiration) fraction driven by the F₁F₀ ATP hydrolysis that is lost because of this dosage. This temperature management issue is more important for smaller than larger animals, because surface area scales to mass by a fractional power (e.g. refer Kleiber's law) and so larger animals retain their generated heat better, and so a given percentage drop in (per unit mass) metabolism will cause a smaller drop in body temperature in a bigger animal. The aforementioned weight gain can be of great clinical/health/nutritional value, or aesthetic value (by non-limiting example: bodybuilders), or commercial value when applied to livestock/farm animals or any animal with a commercial value e.g. racing animals, such as horses. This disclosure encompasses a method/process of using a compound(s) of this disclosure for these applications, or any others wherein weight, nutritional or energetic gain is wanted in an animal or human.

In an embodiment, the amplitude of hypothermia is controlled by setting the ambient temperature, wherein an effective amount of administered F₁F₀ ATP hydrolysis inhibitor reduces subject body temperature to slightly higher than ambient temperature, and so hypothermic amplitude is controlled by controlling ambient temperature. Another embodiment is that the body temperature that the body falls to, upon administration of an effective amount of F₁F₀ ATP hydrolysis inhibitor, is controlled by controlling feature(s) of electromagnetic radiation upon the subject, for example emergent from a radiation heater(s), optionally controlled by servocontrol, with the set point set at the desired hypothermic body temperature, used as a body heating system alone or in combination with other body warming devices and methods (many possibilities known to those of the art), which are optionally controlled by servocontrol, optionally integrated into the same control loop, optionally used by themselves alone or in combination for this body heating purpose, to “catch” and offset the hypothermic drive, of an effective amount of F₁F₀ ATP hydrolysis inhibitor(s) in the body, at some desired hypothermic body temperature.

Componentry to this disclosure is any method in which a subject is administered with an effective amount of a compound(s) of this disclosure, for example a compound(s) of Formula [X], and/or a compound(s) selected from one or more of Formula (I), (II), (III), (IV), (V), (VII), (VIII), and/or any compound(s) that selectively/preferentially inhibits F₁F₀ ATP hydrolysis, and/or a pharmaceutically-acceptable salt, solvate, hydrate or prodrug thereof, and/or a pharmaceutical composition thereof, to reduce their body temperature. For any purpose. Or for no purpose at all. Or to make animals/humans feel more comfortable in hot weather, climates and geographies.

Methods to Find Further Compounds Componentry to this Disclosure

A method to find a compound(s) of this disclosure is by screening for/seeking a compound(s) that preferentially inhibits the reverse mode of ATP synthase. For example, by separately assaying (in space and/or time) a compounds's effect upon ATP synthesis and ATP hydrolysis by ATP synthase (in its entirety or, less preferably, a component part of it). Then comparing these assay results. The greater the inhibition of reverse vs. forward mode, the more preferred a compound is for at least one use of this disclosure. To illustrate, by the teaching of this disclosure, the greater a compound inhibits the reverse vs. forward mode of ATP synthase, the more preferred this compound is for anti-cancer and/or anti-aging use. A disclosure embodiment is the process/method of seeking a new compound(s) of this disclosure by assaying whether a candidate molecule can depolarise Ψ_{IM}, when Ψ_{IM} is maintained by F₁F₀ ATP hydrolysis (e.g. when OXPHOS is blocked by a respiratory chain inhibitor(s) or insufficient O₂), but that can't hyperpolarize Ψ_{IM} and/or decrease O₂ consumption, when Ψ_{IM} is maintained by proton pumping by complexes of the respiratory chain. Such an assay is described in [7]. A further method is screening a number of compounds to find one or more with this activity in this assay. A disclosure embodiment is seeking a compound(s) of this disclosure by assaying whether a candidate molecule inhibits/reduces ATP hydrolysis more than ATP synthesis in Sub-Mitochondrial Particles (SMPs), wherein a further method is screening a number of compounds to find one or more with this activity in this assay. ATP hydrolysis can be assayed by (non-limiting example) a spectroscopic assay for NADH fluorescence that incubates the SMPs with pyruvate kinase and lactate dehydrogenase enzymes (assay well-known to those of the art). ATP synthesis can be assayed by (non-limiting example) a spectroscopic assay for NADPH fluorescence that incubates the SMPs with hexokinase and glucose-6-phosphate dehydrogenase enzymes (assay well-known to those of the art). These assays are reported in in any one of [8, 9, 10, 11, 7, 12, 13], and/or as referenced therein, all of which are herein incorporated in their entirety. In these SMP assays, the criteria for a candidate anti-cancer compound is a low EC₅₀ against ATP hydrolysis (thence anti-cancer activity) and a higher EC₅₀ against ATP synthesis (thence safe for normal cells). These SMP assays deliver high signal-to-noise because non-specific protein inhibiting compounds (Pan-assay interference compounds, PAINS), which are the bane of drug discovery screening assays, inhibit both ATP hydrolysis and synthesis, and thus are dismissed by the screening algorithm. So, the screening assay inherently screens out PAINS. This is distinctive and valuable.

In preferred embodiments, the SMP assay is conducted at alkaline pH (e.g. pH 8). In some embodiments, endogenous/native IF1 protein is removed as a preliminary step of the SMP assay. But in alternative embodiments (more preferred) it is not removed. Which permits the finding of compounds that inhibit F₁F₀ ATP hydrolysis indirectly, by acting upon IF1 protein rather than ATP synthase: by acting to break up IF1 protein tetramers (and higher oligomers; which cannot inhibit F₁F₀ ATP hydrolysis), releasing IF1 protein dimers/monomers, which can inhibit F₁F₀ ATP hydrolysis.

Componentry to this disclosure is screening, using one or more screening assays herein described, compound(s) from one or more compound collections/libraries known to, or findable by, one of the art, optionally a proprietary compound collection(s) {optionally a collection(s) or sub-collection(s) that belongs to, or is sourced from, a major/multinational pharmaceutical company and/or a pharmaceutical company with >$50 million in annual sales and/or a Contract Research Organisation [CRO, illustrative example would be Charles River Laboratories]} and/or a publically/commercially available compound collection(s) (or a fraction thereof), for example, without limitation, eMolecules, Zinc, MMsINCdatabase, Pubchem, Chemspider, chEMBL, Chemical Structure Lookup Service, CoCoCo, Broad Institute compound collection(s), NIH Molecular Libraries Probe Production Centers Network (MLPCN), Joint European Compound library at the European Lead factory, ScreeningPort at Fraunhofer Institute for Molecular Biology and Applied Ecology (IME), Microsource Spectrum collection (contains human approved/trialed drugs), a screening library from Chembridge Inc., San Diego, CA, USA or a similar company (numerous such companies are known to those of the art) and/or by a compound collection/library generated by “diversity-oriented synthesis” and/or by one of the art. A combinatorial library, generated by combinatorial chemistry, may be used, wherein these terms are well known in the art (e.g. refer PCT/US94/08542, EP0774464, U.S. Pat. Nos. 5,798,035, 5,789,172, 5,751,629); and refer to patents with the combinatorial chemistry: sub-class “C40B” in the International Patent Classification; refer GLARE software, available on sourceforge.net website, for combinatorial library design).

Componentry to this disclosure is screening, using one or more screening assays herein described, linear/cyclic (optionally bicyclic, or higher cycle number) peptides using a method(s) of one or more of PCT/US91/08694, PCT/US91/04666, WO2009/098450, U.S. Pat. No. 8,680,022B2, U.S. Pat. No. 9,657,288B2, U.S. Ser. No. 10/501,496B2 or similar, or a method(s) found in a patent application/patent that cites one or more of the aforementioned filings.

A method to find antibody embodiment(s) of this disclosure is to raise antibodies against an ATP synthase component(s), and/or the entirety of ATP synthase, and then assay each in one or more of the aforementioned assays, looking for the ability to preferentially/specifically inhibit F₁F₀ ATP hydrolysis as compared to F₁F₀ ATP synthesis. A disclosure embodiment is to administer a nucleotide sequence coding for such an antibody to a subject, optionally by gene therapy, optionally wherein this antibody coding gene is integrated into the subject's genome in one or more cells, optionally into the subject's mitochondrial DNA (mtDNA) in one or more cells. In an embodiment, one or more antibody embodiments of this disclosure, and/or one or more nucleotide sequences encoding one or more of such antibodies, are administered to a subject to convey to them therapy/enhancement, optionally cancer treatment/amelioration/prevention/combat, optionally wherein one or more of said nucleotide sequences are incorporated into the subject's genome, and/or mitochondrial DNA, in one or more of their cells, optionally wherein the expression of this nucleotide sequence, to protein(s), is limited to a certain cell type/tissue type/organ/area/sub-section of the subject, optionally by the character of the promotor region incorporated with the protein(s) coding sequence and/or by where the sequence is targeted to insert into the genome and/or by where in the subject the nucleotide sequence (optionally in a vector) is introduced and/or by the nature of the vector selected. Incidentally, a disclosure embodiment is for an ATP synthase component(s)/entirety to be administered to a subject, optionally via intravenous administration, wherein this acts as an epitope in the subject, wherein the subject produces antibodies against it, which then convey therapy/enhancement to the subject. What the terms “antibody” and “antibodies” can refer to, and how to produce them (illustrative e.g. refer US2008/0089950A1, Methods and compositions for modulating the immune system and uses thereof, Lan Bo Chen is one of the inventors, also refer to the patents and publications that it cites), is well known in the art and can include, without restriction, monoclonal antibodies, multispecific antibodies, human antibodies, humanized antibodies, camelised antibodies, chimeric antibodies, single domain antibodies, single-chain FVS (ScPv), single chain antibodies, Fab fragments, F(ab′) fragments, disulfide-linked FVs (sdFv), and anti-idiotypic (anti-Id) antibodies, and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The present teaching isn't limited to a particular type of compound. In certain embodiments, a compound of the present teaching can be, but isn't limited to, an inorganic molecule, organic molecule, small organic molecule, small molecule, drug compound, large molecule, nucleic acid, LNA (locked nucleic acid), polynucleotide, oligonucleotide, DNA molecule, gene, protein coding sequence of DNA and/or RNA, plasmid, virus, morpholino, RNA molecule, mRNA, hairpin RNA, siRNA (small interfering RNA), miRNA, antagomir, ribozyme, aptamer, amino acid, amino acid chain, peptide, cyclic peptide, bicyclic peptide, tricyclic (or higher number of cycles) peptide, peptidomimetic, polypeptide, protein, fusion protein, glycopeptide, glycoprotein, antibody, antibody fragment, antibody-drug conjugate, PNA (peptide nucleic acid), lipid, sugar, carbohydrate.

### In Claim Format:

A method of identifying a compound(s)/agent(s) to treat/ameliorate/prevent/combat a disease/disorder/physiological process (and/or one or more of its consequences) selected from: any disease/disorder/physiological process (and/or one or more of its consequences) mentioned herein in this disclosure (in its entirety);
comprising:
independently assaying the compound's effect upon ATP synthesis and ATP hydrolysis by ATP synthase; preferably in vitro; preferably in Sub-Mitochondrial Particles (SMPs); wherein a desired/sought compound inhibits/reduces ATP hydrolysis more than it inhibits/reduces ATP synthesis (by ATP synthase), wherein greater disparity is more preferred; optimally where there is a sizeable differential between a compound's EC₅₀ F₁F₀ ATP synthesis and a smaller valued EC₅₀ F₁F₀ ATP hydrolysis, for example (in order of increasing preference) one or more of >10, >100, >1000, >5000, >10000 times difference; optionally wherein a number of different compounds, optionally from a compound library/libraries, optionally one or more compounds of Formula [X] herein, optionally one or more compounds of one or more of Formula (I), (II), (III), (IV), (V), (VI), (VII), (VIII) herein, are independently tested in this assay, screening/selecting for a compound(s) that inhibits/reduces ATP hydrolysis more than it inhibits/reduces ATP synthesis (by ATP synthase), wherein one or more compounds with this characteristic are selected, especially (but not restrictively) if smaller than 800 Daltons; preferably wherein these selected compounds are ranked according to which have the greatest differential between their EC₅₀ F₁F₀ ATP synthesis and their smaller valued EC₅₀ F1F0 ATP hydrolysis, wherein one or more of the most highly ranked are selected for further optional step(s) of this claim; optionally wherein one or more compounds (and/or analogue/homolog/derivative/salt/solvate/hydrate/prodrug thereof) selected by the aforementioned step is combined with at least one of a pharmaceutically-acceptable carrier(s), additive(s), diluent(s) to produce/manufacture a pharmaceutical composition(s); optionally wherein this selected compound(s)/composition(s) is used to treat/ameliorate/prevent/combat, in a subject (by administering an effective amount), a disease or disorder or physiological process (and/or one or more of its consequences) aforementioned in this claim (i.e. any disease/disorder/physiological process (and/or one or more of its consequences) mentioned herein in this disclosure (in its entirety));
optional use of this selected compound(s)/composition(s) in the manufacture of a medicament to treat/ameliorate/prevent/combat a disease or disorder or physiological process (and/or one or more of its consequences) aforementioned in this claim (i.e. any disease/disorder/physiological process (and/or one or more of its consequences) mentioned herein in this disclosure (in its entirety).

### Yeast Two-Hybrid Screen

[14] built a yeast construct wherein the DNA-binding and transcriptional activator domains of yeast Gal4 transcription factor were divided and associated with Myc and Max. When Myc and Max were free to combine by their known protein-protein interaction, then there was a read out of this association by expression of the β-galactosidase reporter gene. Using this system they screened for compounds that could disrupt the Myc and Max protein-protein interaction. Wherein such a compound stops expression of the β-galactosidase reporter gene (and without it these yeast cannot utilize galactose). In the same way, the DNA-binding and transcriptional activator domains of yeast Gal4 transcription factor can each be associated with an IF1 protein. And this system can then screen for compounds that disrupt IF1 protein dimerization. A compound that can disrupt IF1 protein dimerization in turn prevents IF1 protein tetramerization, therein preventing IF1 protein inactivation by its tetramerization (and higher oligomerization) at pH 8 (normal pH of mitochondrial matrix), wherein an IF1 monomer can (potently) inhibit F₁F₀ ATP hydrolysis. So, this is a screen for compounds that can increase IF1 protein inhibition of F₁F₀ ATP hydrolysis at pH 8 (normal pH of mitochondrial matrix). Note that the yeast nucleus is not at pH 8. But then it doesn't need to be for this screen to work. Because IF1 protein dimerization is not (at least not strongly) pH dependent. Compounds selected by this yeast 2-hybrid screen, which is extremely high throughput, can then be tested in an aforementioned Sub-Mitochondrial Particle (SMP) assay (at pH 8 and wherein endogenous/native IF1 is not depleted beforehand). Wherein if they don't reduce F₁F₀ ATP hydrolysis in this SMP assay, they can be discounted. And, optionally, if they do reduce F₁F₀ ATP hydrolysis, but also reduce F₁F₀ ATP synthesis by a sizeable degree also, they can also be discounted.

Some Cancer Types Especially Targeted by this Teaching

This application discloses a method of using a compound(s) that preferentially inhibits/reduces the ATP-hydrolysing mode of ATP synthase, for example at least one compound of at least one of Formula (I), (II), (III), (IV), (V), (VII), (VIII), [X], or a pharmaceutically-acceptable salt, solvate, hydrate or prodrug thereof, to treat/ameliorate/prevent/combat a cancer, especially a cancer that preferentially uses glycolytic rather than oxidative metabolism, for example a cancer exhibiting the Warburg effect.

A compound of the present disclosure can treat adult cancer, childhood/pediatric cancer, cancer in a child/adolescent, cancer that causes/drives cachexia, cancer occurring/associated with inflammation and/or with Tumour Associated Macrophages (TAMs), chemotherapy and/or radiotherapy and/or immunotherapy resistant/refractory cancer, tumour growth, metastasis, metastatic cancer, non-metastatic cancer, treat a cancer that has spread to the lymph nodes (a “lymph node positive”/“node-positive” cancer), treat a cancer that has not spread to the lymph nodes (a “lymph node negative”/“node-negative” cancer), treat tumour implantation, treat cancer at all clinical stages (e.g. at any stage within stages I-IV, treating pre-cancer in Stage 0 also, e.g. at any stage in the Tumor Node Metastasis [TNM] staging system), treat all grades (e.g. Grades I-III of cancer) of cancer, treat cancer of all degrees of differentiation/de-differentiation/undifferentiation, are useful as an adjunct to chemo-/radio-therapy, treat cancers including, but not limited to, solid tumour/tumor, blood borne tumour/tumor, hematological malignancy, malignancy, advanced malignancy, multiple brain metastase, poor prognosis malignant brain tumor, metastatic hepatocellular carcinoma, hepatocellular carcinoma, liver cancer, primary liver cancer, mesothelioma, malignant melanoma, malignant mesothelioma, malignant pleural effusion mesothelioma syndrome, neuroendocrine tumor, amyloidosis, meningioma, hemangiopericytoma, chondrosarcoma, neurofibroma, Ewing's sarcoma, malignant fibrous histiocytoma of bone/osteosarcoma, osteosarcoma, rhabdomyosarcoma, heart cancer, brain cancer, astrocytoma, neuronal & mixed neuronal-glial tumors, glioma, brainstem glioma, pilocytic astrocytoma, ependymoma, HPV induced/driven/caused/associated/related cancer/tumor, oncogenic DNA virus induced/driven/caused/associated/related cancer, primitive neuroectodermal tumor, craniopharyngioma, cerebellar astrocytoma, cerebral astrocytoma, malignant glioma, recurrent malignant glioma, medulloblastoma, neuroblastoma, schwannoma, oligodendroglioma, anaplastic oligodendroglioma, pineal astrocytoma, anaplastic astrocytoma, pituitary adenoma, visual pathway and hypothalamic glioma, glioblastoma, glioblastoma multiforms, breast cancer, hormone resistant breast cancer, invasive ductal carcinoma, ductal carcinoma in situ (DCIS), invasive lobular carcinoma, tubular carcinoma, invasive cribriform carcinoma, medulloblastoma, medullary carcinoma, male breast cancer, phyllodes tumor, inflammatory breast cancer, adrenocortical carcinoma, islet cell carcinoma, multiple endocrine neoplasia syndrome, parathyroid cancer, pheochromocytoma, thyroid cancer, medullary thyroid carcinoma, papillary thyroid carcinoma, follicular thyroid carcinoma, merkel cell carcinoma, intraocular melanoma, retinoblastoma, ocular neoplasm, anal cancer, appendix cancer, cholangiocarcinoma, carcinoid tumor, colon cancer, extrahepatic bile duct cancer, gallbladder cancer, gastric/stomach cancer, gastrointestinal cancer, gastrointestinal carcinoid tumor, gastrointestinal stromal tumor (GIST), hepatocellular cancer, pancreatic cancer, rectal cancer, bladder cancer, cervical cancer, endometrial cancer, extragonadal germ cell tumor, ovarian cancer, ovarian epithelial cancer (surface epithelial-stromal tumor), ovarian germ cell tumor, uterine cancer, penile cancer, renal cell carcinoma, renal pelvis and ureter, transitional cell cancer, prostate cancer, androgen independent prostate cancer, androgen dependent stage IV non-metastatic prostate cancer, hormone-refractory cancer, hormone-insensitive prostate cancer, hormone resistant prostate cancer, chemotherapy-insensitive prostate cancer, castration-resistant prostate cancer (CRPC), testicular cancer, gestational trophoblastic tumor, ureter and renal pelvis, genitourinary cancer, transitional cell cancer, urethral cancer, uterine sarcoma, vaginal cancer, vulvar cancer, wilms tumor, esophageal cancer, head and neck cancer, nasopharyngeal carcinoma, oral cancer, oropharyngeal cancer, paranasal sinus and nasal cavity cancer, pharyngeal cancer, salivary gland cancer, hypopharyngeal cancer, acute biphenotypic leukemia, acute eosinophilic leukemia, acute lymphoblastic leukemia (ALL), acute myeloid leukemia (AML), chronic myeloid leukemia (CML), acute myeloid dendritic cell leukemia, karotype acute myeloblastic leukemia, primary myelofibrosis, myelodysplastic syndromes (MDS), myeloid sarcoma, myeloproliferative neoplasms (MPNs), lymphoma, AIDS-related lymphoma, anaplastic large cell lymphoma, angioimmunoblastic T-cell lymphoma, B-cell prolymphocytic leukemia, low grade follicular lymphoma, Burkitt's lymphoma, chronic lymphocytic leukemia (CLL), chronic myelogenous leukemia, cutaneous B-Cell lymphoma, cutaneous T-cell lymphoma, diffuse large B-cell lymphoma, follicular lymphoma, hairy cell leukemia, hepatosplenic T-cell lymphoma, Hodgkin's lymphoma, non-Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, mast cell leukemia, mediastinal large B cell lymphoma, multiple myeloma/plasma cell neoplasm, myelodysplastic syndromes, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sézary syndrome, splenic marginal zone lymphoma, T-cell prolymphocytic leukemia, basal-cell carcinoma, melanoma, skin cancer (non-melanoma), bronchial adenomas/carcinoids, small cell lung cancer, mesothelioma, Non-Small Cell Lung Cancer (NSCLC), tobacco-associated NSCLC, pleuropulmonary blastoma, adenocarcinoma, rectal adenocarcinoma, unresectable colorectal carcinoma, laryngeal cancer, thymoma and thymic carcinoma, peritoneal carcinoma, peritoneal cancer, papillary serous carcinoma, AIDS-Related Cancers (ADCs), Kaposi sarcoma, Non-Hodgkin lymphoma (NHL), Burkitt's lymphoma, Burkitt's-like lymphoma, diffuse large B-cell lymphoma (DLBCL), Non-AIDS Related Cancers (NADCs), Hodgkin lymphoma (HL), epithelioid hemangioendothelioma (EHE), desmoplastic small round cell tumor, leiomyoma, leiomyosarcoma, Liposarcoma, fallopian tube cancer, smoldering myeloma, indolent myeloma, Waldenstrom's macroglobulinemia, fibrodysplasia ossificans progressive, breast carcinoma, non-small cell lung carcinoma, ovarian carcinoma, pancreatic carcinoma, prostate carcinoma, colorectal cancer, colorectal carcinoma, squamous cell carcinoma, hepatocellular carcinoma benign prostatic hyperplasia (BPH) and polycystic ovary syndrome, dedifferentiated chordoma, any neoplasm disclosed by the International Classification of Diseases (ICD) in ICD-10 Chapter II: Neoplasms (World Health Organisation, WHO) and/or the International Classification of Diseases for Oncology (WHO).

A compound of the present disclosure can treat cancers including, but not limited to, those that originate in, or spread to, the testis, cerebral cortex, cerebellum, skin, fallopian tube, parathyroid gland, small intestine, large intestine, caecum, kidney, skeletal muscle, muscle, connective tissue, synovium, duodenun, spleen, epididymis, bone, bone marrow, lymphoid, peripheral blood, blood, lymph node, adrenal gland/cortex, esophagus, thyroid gland, heart muscle, tonsil, lung, bronchus, pleura, retroperitoneal, prostate, rectum, anus, adipose tissue, colon, stomach, cervix, gallbladder, seminal vesicle, breast, ovary, endometrium, vulva, smooth muscle, salivary gland, pancreas, urinary bladder, blood, brain, gum, mouth, throat, liver, nasopharynx, other pharynx, pharynx, larynx, neck, tongue, uterus, penis, vagina, chest, eye, retina, head, neck, lip, oral cavity.

A compound of the present disclosure can treat adenomas, carcinomas, leukemias, lymphomas, melanomas, myelomas, sarcomas, and teratomas.

As shown by anti-cancer activity against the cancer cell lines used in NCI-60 testing at the National Cancer Institute (NCI, USA), a compound of this disclosure, which inhibits/reduces F₁F₀ ATP hydrolysis, can treat cancers including, but not limited to, cancer originating in one of peripheral blood, bone marrow, lung, colon, Central Nervous System (CNS), brain, skin, ovary, kidney, prostate, breast/mammary gland; including metastatic forms of these cancers; cancer found in lymph node/bone/soft tissue/metastatic site(s) and/or found in/causing pleural effusion, ascites; Carcinoma, Adenocarcinoma, Squamous cell carcinoma, Large cell carcinoma, Cystadenocarcinoma, Clear cell carcinoma, Sarcoma, Blastoma, cancer of epithelial/fibroblast/promyeloblast/lymphoblast/T lymphoblast/B lymphocyte cell type, Multi Drug Resistant (MDR) cancer, Anaplastic cancer, Hematopoietic cancer, Acute Lymphoblastic Leukemia (ALL), Childhood/Adult T acute lymphoblastic leukemia, Precursor T-cell acute lymphoblastic leukemia, Acute Myeloid Leukemia (AML), Acute promyelocytic leukemia, Chronic Myeloid Leukemia/Chronic Myelogenous Leukemia (CML), CML in blast crisis, Cancer with Philadelphia chromosome (BCR-ABL1 positive), Myeloma, Multiple myeloma, Plasma cell myeloma, Plasmacytoma, Lymphoma, Large cell immunoblastic lymphoma, Anaplastic large cell lymphoma, ALK-positive anaplastic large cell lymphoma, ALK positive cancer, Non-Small Cell Lung Cancer (NSCLC), lung carcinoma, lung adenocarcinoma, Minimally invasive lung adenocarcinoma, Non-small cell lung carcinoma, lung squamous cell carcinoma, Mesothelioma, Pleural epithelioid mesothelioma, Pleural mesothelioma, Bronchioalveolar carcinoma, Large cell lung cancer, Large cell lung carcinoma, Colon carcinoma, Colorectal carcinoma, Colon adenocarcinoma, Colorectal adenocarcinoma, Dukes' type C colorectal adenocarcinoma, Dukes' type D colorectal adenocarcinoma, Astrocytoma, Glioblastoma, Gliosarcoma, Glioblastoma multiforme, Melanoma, Malignant melanoma, Cutaneous melanoma, Amelanotic melanoma, Ovarian adenocarcinoma, Ovarian endometrioid adenocarcinoma, Endometrioid carcinoma of ovary, High grade ovarian serous adenocarcinoma, Ovarian serous cystadenocarcinoma, Renal cell carcinoma, Renal cell adenocarcinoma, Papillary renal cell carcinoma, Clear cell renal cell carcinoma, Clear cell renal carcinoma, Multi Drug Resistant (MDR) renal cancer, Prostate carcinoma, Prostate adenocarcinoma, Androgen Receptor negative (AR−) prostate cancer, Breast carcinoma, Breast adenocarcinoma, Ductal carcinoma, Invasive ductal carcinoma, Luminal A breast cancer, Estrogen Receptor positive (ER+) breast cancer, Progesterone Receptor positive (PR+) breast cancer, Hormone Receptor positive (HR+) breast cancer, Hormone responsive breast cancer, Triple Negative Breast Cancer (TNBC), Hormone Receptor negative (HR−) breast cancer, Hormone resistant breast cancer, Estrogen Receptor negative (ER−) breast cancer, Progesterone Receptor negative (PR−) breast cancer, HER2 negative (HER2−) breast cancer.

Local Administration of a Compound(s) of this Disclosure, Optionally for Cancer Treatment

In some disclosure embodiments a compound(s) of this disclosure, optionally a compound(s) of at least one of Formula (I), (II), (III), (IV), (V), (VI), (VII), (VIII), [X], or a pharmaceutically-acceptable salt, solvate, hydrate or prodrug thereof, is administered to a subject locally rather than systemically, optionally to convey therapy, optionally to treat/ameliorate/prevent/combat cancer in a subject, optionally wherein the local administration is to the cancer(s) itself. For non-limiting example, wherein the local administration is to a skin cancer(s) and/or pre-cancer, optionally basal-cell skin cancer (BCC), squamous-cell skin cancer (SCC), melanoma, dermatofibrosarcoma protuberans, Merkel cell carcinoma, Kaposi's sarcoma, keratoacanthoma, spindle cell tumor, sebaceous carcinoma, microcystic adnexal carcinoma, Paget's disease of the breast, atypical fibroxanthoma, leiomyosarcoma, angiosarcoma, hemangioma, Melanocytic nevus, Bowen's disease, Actinic keratoses, optionally administered via a liquid/solution/cream/lotion/ointment/emulsion/foam/spray/patch/transdermal patch/adhesive bandage/time release technology or some other drug administration route known to one of the art. Skin cancer is the most prevalent cancer globally. This local drug administration can locally reduce F₁F₀ ATP hydrolysis, thence F₁F₀ ATP synthesis, oxidative phosphorylation rate and metabolic heat generation, which is not detrimental when ambient temperature is 37° C., and not detrimental when ambient temperature is lower because heat transfer from the rest of the body, especially via blood flow, maintains the drug administered area at or near 37° C.

In a disclosure embodiment, one or more F₁F₀ ATP hydrolysis inhibitors of this disclosure, or a pharmaceutically-acceptable salt, solvate, hydrate or prodrug thereof, are administered to a subject topically/locally rather than systemically, optionally to a cancer(s) or close to a cancer(s) or to a blood vessel perfusing a cancer(s), wherein this cancer can be a tumour, and thence the compound(s) conferred reduction in heat generation (and slower aging) is disproportionally applied to this localized region, wherein its lesser heat generation is offset by heat transfer from surrounding body area(s), especially given the heat distributing nature of blood flow. In a particular embodiment, the cancer is suspected rather than diagnosed. In an embodiment, a compound(s) of this disclosure is applied topically to the skin, optionally to a skin cancer(s).

### Aging

An embodiment is a method in which a subject takes or is administered an effective amount of a compound(s) of this disclosure, for example at least one compound of at least one of Formula (I), (II), (III), (IV), (V), (VII), (VIII), [X], and/or another compound(s) that selectively inhibits F₁F₀ ATP hydrolysis, and/or a pharmaceutically-acceptable salt, solvate, hydrate or prodrug thereof, to slow their aging and/or delay the onset and/or delay/slow the progression of an age-related disease(s) and/or condition(s) and/or to extend the lifespan (and/or healthspan) of the subject (e.g. relative to the lifespan of a control subject(s) {optionally a mean/median/mode of a number of control subjects} of the same species), and/or to treat/ameliorate/prevent/combat an accelerated aging disease or progeroid syndrome. It is to be understood that “age-related” refers to diseases/disorders/conditions frequently associated with aging, however, a given subject need not be of advance age, but rather the methods, compounds and compositions of this disclosure can be used regardless of the subject's age.

Not only does an F₁F₀ ATP hydrolysis inhibitor compound of this disclosure treat/ameliorate/combat cancer in a subject, it also prevents cancer in a subject, which is distinct from many other cancer treatments (e.g. radiotherapy) which are a drive to further cancer, and so compounds of this disclosure are especially preferred for cancer treatment in children (pediatric cancers), who have enough lifespan left for secondary cancers, as a result of radiotherapy for example 1151, to be a very severe concern. Also it is noteworthy that compounds of this disclosure both treat cancer and slow aging, whereas many present cancer treatments accelerate aging 1161, causing greater incidence of age related disease(s) and ailments.

An anti-aging compound does one or more of slowing/reversing aging, slowing/reversing a sign(s) of aging, extending lifespan and/or healthspan, delaying/preventing/treating one or more diseases that have an increased incidence with age (such as the neurodegenerative diseases), treating accelerated aging diseases. Any anti-aging compound that targets/inhibits F₁F₀ ATP hydrolysis is componentry to this disclosure, preferably those that preferentially inhibit F₁F₀ ATP hydrolysis as compared to F₁F₀ ATP synthesis, and most preferably those that don't inhibit F₁F₀ ATP synthesis at all. This application discloses numerous such drug examples, many of which are also new compositions of matter, and discloses rationale and methods to find further drug examples (e.g. SMP studies, looking for compounds that inhibit F₁F₀ ATP hydrolysis more than F₁F₀ ATP synthesis), which are, in turn, encompassed and componentry to this disclosure, for example for an anti-aging use, or for other disclosed use(s) herein.

### Anti-Aging Skin Cream

F₁F₀ ATP hydrolysis inhibitor compound(s) of this disclosure slow aging but can reduce body temperature. A disclosure embodiment is to target an F₁F₀ ATP hydrolysis inhibitor compound(s) to a part/area of the subject/body where slower aging is desired, optionally for aesthetic/cosmetic or medical/therapeutic desire or need. This body part or area will have slower aging and lesser heat production, but heat transfer from surrounding body areas (especially via blood flow) will maintain the temperature of this body part/area at an acceptable value. So, the temperature issue is mitigated and slower aging endures in that body part/area. A disclosure embodiment is a method in which a subject takes or is administered an effective amount of a compound(s) of this disclosure, for example at least one compound of at least one of Formula (I), (II), (III), (IV), (V), (VII), (VIII), [X], and/or another compound(s) that selectively inhibits F₁F₀ ATP hydrolysis, or a pharmaceutically-acceptable salt, solvate, hydrate or prodrug thereof, to treat/ameliorate/prevent/combat skin aging, optionally administered to the skin, optionally by skin and/or subcutaneous injection/implant, optionally as a skin cream, optionally to the face. In another embodiment, administered to the scalp and/or hair, optionally in a hair treatment, optionally in a shampoo, to treat/ameliorate/prevent/combat hair follicle and hair aging/loss/greying/baldness. All means of applying a compound(s) of this disclosure to the skin, and/or scalp and/or hair are contemplated by, and componentry to, this disclosure.

Some Cosmetic/Aesthetic Embodiments of this Disclosure

A F₁F₀ ATP hydrolysis inhibitor compound(s), optionally at least one compound of at least one of Formula (I), (II), (III), (IV), (V), (VI), (VII), (VIII), [X], and/or a salt, solvate, hydrate, prodrug, precursor, liposome, nanoparticle (e.g. lipid nanoparticle, LNP) or other vector of the art thereof, and/or a pharmaceutical/cosmetic composition/formulation thereof, as the entirety, or as at least one component/ingredient, of a cosmetic;

For non-limiting example, a cosmetic wherein one or more of the following features apply to it (all combinations contemplated except those that are mutually exclusive):

Optionally wherein the cosmetic also contains:
one or more ingredients in a cosmetic for sale in the USA/Canada/European Union/Japan/China/Korea/Australia/Brazil,
and/or one or more ingredients in a cosmetic made by a top 100 (by market cap/sales) multinational cosmetic company/conglomerate,
and/or one or more ingredients in a cosmetic(s) made/marketed by one or more of Sederma SAS (France), Lipotec SA (Barcelona, Spain), L'Oréal, Unilever, Estée Lauder, Proctor and Gamble, Coty, Shiseido, Beiersdorf, Johnson & Johnson, Amore Pacific, Kao Corporation, Colgate-Palmolive, Chanel, Revlon, or similar,
and/or one or more ingredients in a commercially available cosmetic(s) that has one or more peptide ingredients,
and/or one or more ingredients listed in the International Nomenclature of Cosmetic Ingredients (INCI, INCI names are developed by the International Nomenclature Committee, INC) and/or included in the CTFA “International Cosmetic Ingredient Dictionary and Handbook” [wherein CTFA is “Cosmetic, Toiletry, and Fragrance Association, Inc.”, Washington, DC, USA],
and/or one or more ingredients listed in CosIng (the European Commission database for information on cosmetic substances and ingredients),
and/or one or more cosmetic ingrediants taught by one or more of: (i) “Harry's Cosmeticology” [e.g. its 9^{th} edition by Meyer R. Rosen (Editor)], Chemical Publishing Company, USA, (ii) Milady's “Standard Textbook of Cosmetology” (Delmar Learning), (iii) “Formulation Technology. Emulsions, Suspensions, Solid Forms” by Hans Mollet, Arnold Grubenmann and Helen Payne, published by John Wiley & Sons, (iv) “Chemistry and Technology of the Cosmetics and Toiletries Industry” by Clifford Williams Schmitt, Kluwer Academic Publishers, (v) Fiedler's “Encyclopedia of Excipients”, Cantor Verlag Aulendorf,
and/or one or more ingrediants listed in U.S. Pharmacopeia (USP)₂₅-NF20 (2002),
and/or one or more pharmaceutical/cosmetic ingredients/vehicles/carriers/additives/diluents/excipients/adjuvants/active agents (especially, but not restrictively, to those pertaining to treatment, health and/or care of the skin, including those increasing percutaneous absorption of peptides) listed in U.S. Pat. No. 8,946,166B2 (and/or in one or more of U.S. Pat. No. 9,067,967B2, U.S. Pat. No. 9,315,564B2, US2013/0078295A1, US2014/0322307A1, WO2014/170347A1, U.S. Pat. No. 6,372,717B1, U.S. Pat. No. 6,620,419B1, U.S. Pat. No. 6,974,799B2, U.S. Pat. No. 7,182,963B2, U.S. Pat. No. 7,998,493B2, U.S. Pat. No. 8,404,648B2, U.S. Ser. No. 10/660,839B2, U.S. Ser. No. 10/668,000B2, U.S. Ser. No. 10/668,000B2, US2004/0132667A1, US2018/0000717A1, WO2019149450A1, WO00/62743, U.S. Pat. No. 7,863,417B2, U.S. Pat. No. 7,671,009B2, and references therein),
and/or one or more ingredients commonly used in compositions for the treatment and/or care of the skin,
and/or one or more anti-aging/anti-wrinkle compounds/ingredients/agents: e.g., without limitation, botulinum toxin, epidermal growth factor, rapamycin, Vitamin A, one or more Retinoids {e.g., without limitation, retinol, retinal, tretinoin [all-trans-retinoic acid], isotretinoin [13-cis-retinoic acid], alitretinoin [9-cis-retinoic acid], etretinate, acitretin, adapalene, bexarotene, tazarotene, seletinoid G}, one or more retinoid complexes/salts/esters/ethers (e.g., without limitation, Retinyl palmitate), one or more Alpha Hydroxy Acids [AHAs], one or more Beta Hydroxy Acids [BHAs], Vitamin C, Vitamin E, Coenzyme Q10, one or more antioxidants, one or more peptides (e.g., without restriction, acetyl hexapeptide-3, acetyl hexapeptide-8, Matrixyl™ [palmitoyl pentapeptide-4], OS-01 peptide from company called OneSkin, heptapeptide-7, one or more defensins), one or more copper peptides e.g. copper peptide GHK-Cu, Matryxil, Nicotinamide adenine dinucleotide (NAD⁺), Nicotinamide mononucleotide [NMN], Nicotinamide riboside (NR), Nicotinamide (Nam), Nicotinic acid (NA), Nicotinic acid adenine dinucleotide (NaAD), Nicotinic acid mononucleotide (NaMN), platelet-rich plasma, rapamycin),
and/or one or more compounds/ingredients to treat/ameliorate/prevent/combat hair loss: e.g., without limitation, one or more 5a-Reductase inhibitors {5-ARIs, also known as dihydrotestosterone (DHT) blockers}, Finasteride, Dutasteride, Epristeride, Saw palmetto extract, Serenoa repens extract, Alfatradiol (also known as 17a-estradiol), one or more antiandrogens (e.g., without limitation, steroidal antiandrogens and non-steroidal antiandrogens), Bicalutamide, Bimatoprost, Cyproterone acetate, Flutamide, Ketoconazole, Latanoprost, Minoxidil, MK-434, Nepidermin, Nonsteroidal antiandrogen, RU-58841, Spironolactone, Steroidal antiandrogen, Topilutamide, Kopexil, Latanoprost, bimatoprost, Pinacidil, Diazoxide, one or more corticosteroids, IGF-1 (optionally in liposomes),
and/or one or more natural products (e.g. plant, marine, tissue) extracts,
and/or one or more of Acetone, Acetyl hexapeptide-3, Allantoin, Aloe, Alpha hydroxy acid, Aluminium zirconium tetrachlorohydrex gly, Argan oil, Azulene, Behentrimonium chloride, Bimatoprost, Bisabolol, Canthaxanthin, Carnauba wax, Castor oil, Ceteareth, Cetyl alcohol, Cocamide DEA, Cocamide MEA, Cocamidopropyl betaine, Cocamidopropyl hydroxysultaine, Cocoa butter, Conditioner, Copernicia Cerifera (Carnauba) Wax, Copper peptide GHK-Cu, Decamethylcyclopentasiloxane, Dihydroxyacetone, Dioxalin, Dipropylene glycol, Disodium cocoamphodiacetate, DMDM hydantoin, Erythrulose, Ethyl macadamiate, Ethylhexyl palmitate, Film-forming agent, Glycerol, Glyceryl behenate, Glycol distearate, Guaiazulene, Guanine, Hydrogenated jojoba oil, Hydrolyzed jojoba ester, Iodopropynyl butylcarbamate, Isoceteth-20, Isopropyl jojobate, Isopropyl myristate, Isopropyl palmitate, Jojoba alcohol, Jojoba ester, Jojoba oil, Jojoba Wax PEG-80 Esters, Jojoba Wax PEG-120 Esters, Lapyrium, Macadamia oil, Malic acid, Marula oil, Microbead, Microcrystalline wax, Mineral cosmetics, Mineral oil, Myristamine oxide, Oleyl alcohol, denatured alcohol, Palmitoyl pentapeptide-4, Panthenol, Paraben, PEG-10 sunflower glycerides, PEG-16 macadamia glycerides, PEG-80 Jojoba, PEG-120 Jojoba, PEG-150 hydrogenated jojoba, Petroleum jelly, Polyacrylic acid, Polydimethylsiloxane, Polyethylene glycol propylene glycol cocoates, Polyquaternium, Polyquaternium-7, Propylene glycol, Quaternium-15, Rice bran wax, Sculptra, Selenium disulfide, Silicone, Simmondsia Chinensis (Jojoba) Seed Oil, Simmondsia chinensis (jojoba) seed powder, Sodium laureth sulfate, Sodium lauroamphoacetate, Sodium lauroyl sarcosinate, Sodium myreth sulfate, Spermaceti, Stearalkonium chloride, Stearamidopropyl dimethylamine, Sunflower oil, Talc, 1-Tetradecanol, Tetramethyl acetyloctahydronaphthalenes, Tocopherol, 1-Tridecanol, Triethanolamine, Vitellaria, Zinc pyrithione, Zinc ricinoleate, Amiloxate, 4-Aminobenzoic acid, Avobenzone, Bemotrizinol, Benzophenone-n, Bisdisulizole disodium, Bisoctrizole, Cerium(IV) oxide, Cinoxate, Dibenzylideneacetone, Diethylamino hydroxybenzoyl hexyl benzoate, Dioxybenzone, Drometrizole trisiloxane, Ecamsule, Ensulizole, Enzacamene, Ethylhexyl triazone, Homosalate, Iscotrizinol, Menthyl anthranilate, Mexenone, Octocrylene, Octyl methoxycinnamate, Octyl salicylate, Oxybenzone, Padimate A, Padimate O, Polysilicone-15, Sulisobenzone, Titanium dioxide, Titanium dioxide nanoparticle, Trolamine salicylate, Umbelliferone, Zinc oxide, alkyl benzoate C12-C15, allantoin, water, ascorbyl palmitate, butane, Butyrospermum parkii, shea butter, cocoamide dea, dodecanol, egg oil, hydroxyethyl cellulose, hydroxypropyl cellulose, isobutane, isopentane, lauryl glucoside, Polysorbate 20, propane, sodium hydroxide, triethanolamine, honey, shea butter, almond oil, argan oil, rosehip oil, beeswax, stevia, glycerin, essential oil(s), benzyl alcohol, dehydroacetic acid, Glyceryl Caprilate, Potassium sorbate, Caprylhydroxamic Acid, Caprylyl Glycol, Glycerin, Xanthan Gum, EDTA, Emulsifying wax, Olive oil, Evening primrose oil, Tocopheyl Acetate, Cetearyl Alcohol, Caprylyl Glycol, Phenoxyethanol, Hexylene Glycol, Glycyrrhiza Glabra (Licorice) Root Extract, Sodium Hyaluronate, oleic pau mulato extract, pracaxi oil, allantoin, niacinamide, hyaluronic acid, andiroba oil,
and/or one or more compounds conferring one or more of the following activities: anti-aging, anti-wrinkle, rejuvenating, moisturizing/hydrating, revitalizing, conditioning, skin restructuring, skin relaxing, oily skin treatment, for reducing sebum production by sebocytes, exfoliating, anti-oxidant, free radical scavenging, pigment, colourant, skin whitening, skin tanning, anti-microbial, anti-bacterial, anti-fungal, anti-parasitic, antipsoriasis agents, anti-hair loss, hair growth induction, anti-cellulite, anti-stretch mark, anti-scar, anti-acne, anti-spot, anti-eczma, anti-dermatitis, perfume, anti-persperant, lubricant, anti-itch, anti-inflammatory, anti-histamine, DNA repair/protect, wound healing, epidermic hydrolytic enzyme, sun (UVA and/or UVB) protection;
optionally wherein the cosmetic is intended to be rubbed/poured/sprinkled/sprayed on/introduced into or otherwise applied to the human body;
for example (to illustrate, and NOT restrict) a skin and/or hair care product, shampoo, antidandruff shampoo, conditioner, a product delivered by micro-needling/dermaroller/plasma-needling/DermaPen, hair tonic, hair colour, hair dye, soap, soap substitute, shower gel, bath oil, bubble bath, toothpaste, mouthwash, moisturizer, emollient, face cream, eye cream, skin cream, skin/face/body/hair/moisturizing/anti-aging/anti-wrinkle/crow's-feet/masking/whitening/tanning/age spot/liver spot cream/oily cream/aqueous cream/lotion/powder/spray/aerosol/butter/gel/hydrogel/oil/salve/liquid/alcohol/emulsion/anhydrous-cream/stick/wax/ointment/foam/paste/solution/drop/gum/jelly/serum/scrub/mask/balm, stretch mark/cellulite/thigh cream/treatment, varicose vein cream, chapstick, lipstick, lip protector, lip gloss, lip liner, lip plumper, lip balm, lip stain, lip conditioner, lip primer, lip booster, lip butter, make-up, makeup, make-up foundation (such as fluid/compact foundation), rouge, make-up removal lotion/milk, under-eye concealer, under-eye cream, eye cream (for application around the eyes), eye shadow, mascara, mascara primer, eye shadow, eye liner, eyebrow pencil/cream/wax/gel/powder, foundation, concealer, bronzer, fake tan, complexion enhancer, rouge, blush, blusher, highlighter, setting spray, cleanser, skin cleanser, foaming wash, toner, eye mask, facial mask (non-limiting e.g. clay-based masks, e.g. using kaolin clay or fuller's earth, peel masks, sheet masks), exfoliant, perfume, cologne, aftershave, shaving foam, beard balm, fragrance, deodorant, antiperspirant, hairstyling product(s), hairspray, hair dye, nail polish, massage oil, barrier cream, sunscreen/sunblock/sun cream (e.g. offering protection against UVA and/or UVB radiation), spot/acne cream;
optionally wherein the cosmetic is for one or more of cleansing, beautifying, promoting attractiveness and/or altering the appearance of a subject (e.g., without restriction, making a more youthful/younger appearance, reducing the appearance of lines and wrinkles, preventing/reducing the sign(s) of aging/premature aging, making skin/hair look visibly younger).

Componentry to this disclosure is a compound of Formula (VII) or (VIII), e.g. at least one IF1 protein/fragment (e.g. from a human and/or another mammalian species, or sequence variant thereof), in liposomes (or lipid nanoparticles) in a fluid gel formulation (e.g., without limitation, as used in 1171 for (IGF)-1), optionally in use as a cosmetic, optionally wherein a more youthful/younger appearance is desired in a subject.

Both therapeutic and/or non-therapeutic use of a cosmetic of this disclosure is componentry to this disclosure.

Skin (e.g. Facial Skin) Administration, Including for Cosmetic Purpose(s)

A teaching/ingredient(s)/vehicle(s)/carrier(s)/additive(s)/diluent(s)/excipient(s)/adjuvant(s)/active agent(s) of a pharmaceutical/cosmetic composition (especially, but not restrictively, for skin administration) in U.S. Pat. No. 8,946,166B2 (and/or in one or more of U.S. Pat. No. 9,067,967B2, U.S. Pat. No. 9,315,564B2, US2013/0078295A1, US2014/0322307A1, WO2014/170347A1), but as applied to/implemented for/combined with a compound(s) of the present disclosure, is pharmaceutical/cosmetic composition of the present disclosure.

A compound(s), and/or cosmetic/pharmaceutical composition thereof, of this disclosure can be applied to the skin (e.g. facial skin) by iontophoresis, sonophoresis, electroporation, microelectric patch(es), mechanical pressure, osmotic pressure gradient, occlusive cure, microinjection(s), needle-free injection(s) by means of pressure, such as injection(s) by oxygen pressure, or any combination thereof.

At least one compound of this disclosure, and/or at least one cosmetic/pharmaceutical composition thereof, optionally for topical/transdermal application, can be produced in any solid, liquid or semi-solid formulation, for example, and not restricted to, one or more of (or combination thereof) cream, multiple emulsion (for example, and not restricted to, oil and/or silicone in water emulsion, water-in-oil and/or silicone emulsion, water/oil/water or water/silicone/water type emulsion, and oil/water/oil or silicone/water/silicone type emulsion), anhydrous composition, aqueous dispersion, oil, milk, balsam, foam, lotion, gel, cream gel, hydroalcoholic solution, hydroglycolic solution, hydrogel, liniment, sera, soap, shampoo, conditioner, serum, polysaccharide film, ointment, mousse, pomade, powder, bar, pencil, spray, aerosol (spray), including leave-on and rinse-off formulations. These topical/transdermal application formulations can be incorporated, using techniques known by a person of the art, into different make-up products such as one or more of make-up foundation (such as fluid foundations and compact foundations), make-up removal lotion, make-up removal milk, under-eye concealer, eye shadow, lipstick, lip protector, lip gloss and powder among others. These topical/transdermal application formulations can be incorporated, using techniques known by one of the art, into a fabric, non-woven fabric, medical device, which is in direct contact with the skin (optionally which can release active agent(s) by biodegradation of the binding system to the fabric, non-woven fabric or medical device, or by the friction between them and the body, and/or due to one or more of body moisture, the skin's pH, body temperature etc.), for example into different types of solid accessories for example, and not restricted to one or more of bandage, gauze, t-shirt, socks, tights, underwear, girdle, gloves, diaper, sanitary napkin, dressing, bedspread, wipes, adhesive patch, non-adhesive patch, occlusive patch, micro-electric patch or face mask.

In particular embodiments, at least one compound of/in this disclosure, optionally in a cosmetic/pharmaceutical composition, can be adsorbed on one or more of a solid organic polymer, solid mineral carrier such as, but not limited to, talc, bentonite, silica, starch or maltodextrin, among others.

### Eye Aging

F₁F₀ ATP hydrolysis inhibitor compound(s) of this disclosure slow aging but can reduce body temperature. A disclosure embodiment is to target an F₁F₀ ATP hydrolysis inhibitor compound(s) to one or both eyes of a subject, optionally by intravitreal injection(s) and/or eye drop(s) and/or contact lens coating/solution (optionally wherein the contact lens has little to no refractive ability or wherein the contact lens is prescriptive to the refractive defect/error of the subject's eye[s]) and/or some other drug administration route/device to the eye(s), known or findable to those of the art, wherein the eye(s) then has slower aging and lesser heat production, but wherein heat transfer from surrounding body areas (especially via blood flow) maintains eye(s) temperature at acceptable value. So, the temperature issue is mitigated and slower aging in the eye(s) endures. An embodiment is a method in which a subject takes or is administered an effective amount of a compound(s) of this disclosure, for example at least one compound of at least one of Formula (I), (II), (III), (IV), (V), (VII), (VIII), [X], and/or another compound that selectively inhibits F₁F₀ ATP hydrolysis, or a pharmaceutically-acceptable salt, solvate, hydrate or prodrug thereof, optionally by a local drug administration route to the eye(s) (e.g. by an eye delivery route known or findable to those of the art e.g. an eye(s) administration route used for an FDA/EMA licensed/sanctioned drug(s)/treatment(s) e.g. as described in the patent/scientific literature e.g. refer [18, 19, 20] and the papers they cite and the papers that cite them, e.g. refer U.S. Pat. No. 8,729,010B2 and references therein, which also teaches pharmaceutical compositions for eye delivery), to treat/ameliorate/prevent/combat eye(s) aging and/or an eye aging related disease/disorder, including any eye disease/disorder whose likelihood of onset increases with age and/or worsens with age, including, without limitation, age-related macular degeneration (AMD, early/intermediate/late), age-related wet macular degeneration, neovascular/wet AMD, dry AMD, Geographic atrophy (GA), wet and dry AMD in the same eye(s), Stargardt's macular degeneration, Best vitelliform macular dystrophy, diabetic retinopathy, proliferative diabetic retinopathy, diabetic macular edema, vision loss, progressive vision impairment, myopia (short-sightedness), degenerative myopia, hyperopia (far-sightedness), accommodative dysfunction, glaucoma, progressive glaucoma, cataract formation, retinal degeneration, progressive retinal degeneration, retinitis pigmentosa, leber hereditary optic neuropathy, Fuchs spot, Best's disease, Sorsby's fundus dystrophy. In an embodiment, one eye of the subject is treated and the other not (optionally administered drug vehicle control), optionally for a course of administrations over a period of time, and the anatomical/physiological/functional difference(s) between them is then compared after some period. Non-limiting example eye function tests are using the Snellen chart, or LogMAR chart, for visual acuity testing and/or the Amsler grid to investigate central vision. In an embodiment, a subject genetically predispositioned to age-associated eye disease(s)/disorder(s), e.g. macular degeneration, optionally discovered by genetic testing and/or family history analysis, is administered a compound(s) of this disclosure prophylactically. When a compound(s) of this disclosure is administered by intravitreal injection(s), optionally antibiotic(s) eye drop(s) (alternatively/in addition oral antibiotic(s)) is administered one or more times on the same day and/or in the same week and/or in the same month. Given increasingly aging societies in many countries, more and more people are succumbing to macular degeneration. It is noteworthy that there is presently no treatment on the market for dry AMD, which comprises 90% of macular degeneration cases, affecting millions globally. The projected number of people with age-related macular degeneration in 2020 is 196 million, increasing to 288 million in 2040 [21].

### Ear Aging

F₁F₀ ATP hydrolysis inhibitor compound(s) of this disclosure slow aging but can reduce body temperature. A disclosure embodiment is to target an F₁F₀ ATP hydrolysis inhibitor compound(s) to one or both ears of a subject, optionally by intratympanic and/or intracochlear administration and/or trans-oval window delivery and/or by ear drops and/or some other drug administration route/device to the ear(s), known or findable to those of the art, wherein the ear(s) then has slower aging and lesser heat production, but wherein heat transfer from surrounding body areas (especially via blood flow) maintains ear(s) temperature at acceptable value. So, the temperature issue is mitigated and slower aging in the ear(s) endures. A disclosure embodiment is a method in which a subject takes or is administered an effective amount of a compound(s) of this disclosure, for example at least one compound(s) of at least one of Formula (I), (II), (III), (IV), (V), (VII), (VIII), [X], and/or another compound(s) that selectively inhibits F₁F₀ ATP hydrolysis, and/or a pharmaceutically-acceptable salt, solvate, hydrate or prodrug thereof, optionally by a local drug administration route to the ear(s) (e.g. by an ear delivery route known or findable to those of the art e.g. an ear(s) administration route used for an FDA/EMA licensed/sanctioned drug(s)/treatment(s) e.g. as described in the patent/scientific literature e.g. refer 122, 231 and the papers they cite and the papers that cite them), to treat/ameliorate/prevent/combat ear(s) aging and/or an ear aging related disease/disorder, including any ear disease/disorder whose likelihood of onset increases with age and/or worsens with age, including, without limitation, age-related hearing loss, presbycusis, tinnitus.

Joint (e.g. Knee) Aging

F₁F₀ ATP hydrolysis inhibitor compound(s) of this disclosure slows aging but can reduce body temperature. A disclosure embodiment is to target/administer/apply an F₁F₀ ATP hydrolysis inhibitor compound(s) to one or more joints (optionally an osteoarthritic joint[s]) of a subject, e.g. one or both knees (optionally an osteoarthritic knee[s]), administered directly into the joint(s), optionally administered intra-articularly to the joint(s), optionally administered intra-articularly to an osteoarthritic joint(s), optionally administered topically/transdermally/intradermally to the (optionally osteoarthritc) joint(s)/knee(s) wherein the joint(s) then has slower aging and lesser heat production, but wherein heat transfer from surrounding body areas (especially via blood flow) maintains joint(s) temperature at acceptable value. So, the temperature issue is mitigated and slower aging in the joint(s) endures. An embodiment is a method in which a subject takes, or is administered, an effective amount of a compound(s) of this disclosure, for example at least one compound(s) of at least one of Formula (I), (II), (III), (IV), (V), (VII), (VIII), [X], and/or another compound(s) that selectively inhibits F₁F₀ ATP hydrolysis, and/or a pharmaceutically-acceptable salt, solvate, hydrate or prodrug thereof, optionally by local administration to a joint[s](e.g. injection into a joint[s], e.g. a joint[s] administration route used for an FDA/EMA licensed/sanctioned drug[s]/treatment[s], e.g. as described in the patent/scientific literature), to treat/ameliorate/prevent/combat joint(s) aging and/or a joint aging related disease/disorder/condition, including any joint disease/disorder/condition/pain whose likelihood of onset increases with age and/or worsens with age, including, without limitation, osteoarthritis. All joints are hereby contemplated, including, to illustrate and not restrict, knee(s) and/or elbow(s) and/or wrist(s) and/or shoulder(s) and/or ankle(s) and/or hip(s) and/or one or more joints of the hand(s) and/or foot/feet.

### Brain Aging

Neurodegenerative diseases have an aging component to their etiology [24] as their onset is a function of age (oxidative stress [24]). Indeed, all these diseases (prototypical examples include Parkinson's disease, dementia, Alzheimer's disease, amyotrophic lateral sclerosis {ALS}, Huntington's disease, Friedreich's ataxia, hereditary spastic paraplegia) can be thought of as the brain aging faster and dying before the rest of the body (adult brain mass decreases with age [25]). In our rapidly greying societies these diseases are a demographic time bomb. Indeed, beyond immeasurable personal suffering, they stand to decimate whole economies (healthcare spending becomes unsustainable percentage of GDP, already ˜30% in the USA). For example, nearly half of Americans, over 85, have dementia, which in time is an age that an increasing proportion of the population will surpass, it has no cure and can be completely debilitating, which strains families and communities [25]. Thus, any treatment that can slow brain aging, to make brain function last as long as the rest of the body, will greatly assist in matching “healthspan” to lifespan, which is arguably the Holy Grail in modern medicine.

A disclosure embodiment is a method in which a subject takes or is administered an effective amount of a compound(s) of this disclosure, for example at least one compound of at least one of Formula (I), (II), (III), (IV), (V), (VII), (VIII), [X], and/or another compound that selectively inhibits F₁F₀ ATP hydrolysis, and/or a pharmaceutically-acceptable salt, solvate, hydrate or prodrug thereof, to treat/ameliorate/prevent/combat brain aging and neurodegenerative disease(s). Optionally wherein the compound(s) is disproportionally delivered to the brain or central nervous system (CNS), or to specific brain/CNS area(s) or cell type(s), by administration route, strategy or targeting. Illustratively, not restrictively, brain targeting had been shown with exogenous dopamine [26-27]. Preferred brain structures/cells/neurons to target are those whose failure drives a neurodegenerative disease e.g. dopamine neurons in the pars compacta (in the substantia nigra). There are few of them, only 7,200 in rat [28], and in humans their number decline by aging at 5-10% per decade [29], which is a predisposing drive to Parkinson's disease (PD). A disclosure embodiment is a method in which a subject takes or is administered an effective amount of a compound(s) of this disclosure, for example at least one compound of at least one of Formula (I), (II), (III), (IV), (V), (VII), (VIII), [X], and/or another compound that selectively inhibits F₁F₀ ATP hydrolysis, or a pharmaceutically-acceptable salt, solvate, hydrate or prodrug thereof, to treat/ameliorate/prevent/combat Parkinson's disease, optionally wherein the compound(s) is disproportionally administered to dopamine neurons in the substantia nigra. If a compound of this disclosure decreases their heat generation, heat transfer from neighbouring brain and/or body regions will substitute this heat.

Lifespan and/or Healthspan Extension in a Subject by Administrating A Compound(S) of this Disclosure
Maximal Tolerated Dose (MTD) Study of a Compound(s) of this Disclosure

Three mice receive an initial dose of intravenous (i.v.) 10 mg/kg of drug. If these mice survive for 72 hours, the i.v. dose for the next cohort of three different mice is increased, whereas if one or more mice die, the i.v. dose for the next cohort of three different mice is decreased. And this schema is run iteratively. To illustrate, next dose level, 72 hours after prior dose level, can be determined by the following scheme:
10 mg/kg, if no death, 30 mg/kg, if no death, 100 mg/kg
10 mg/kg if no death, 30 mg/kg, if death, 17 mg/kg
10 mg/kg, if death, 3 mg/kg, if death, 1 mg/kg,
10 mg/kg, if death, 3 mg/kg, if no death, 5 mg/kg

But other schema can be developed by one of the art, involving different doses and/or a greater number of doses and/or using a different route of administration e.g. oral (PO), intraperitoneal (IP), intravenous (IV), subcutaneous (SC), intramuscular (IM) or other. At each dose level, animals are observed for the presence of acute toxic symptoms (mortality, convulsions, tremors, muscle relaxation, sedation, etc.) and autonomic effects (diarrhea, salivation, lacrimation, vasodilation, piloerection, etc.) during the first 60 minutes, again at 2, 24, 48 and 72 hours. Body weights are recorded pre-dose and at 72 hours after dose.

An alternative MTD determination method that better conserves compound and minimizes the number of animals sacrificed: a single mouse is given a dose (IP, IV, SC, IM or PO) of 400 mg/kg, a second mouse receives a dose of 200 mg/kg and a third mouse receives a dose of 100 mg/kg. The mice are observed for a period of 2 weeks. They are sacrificed if they lose more than 20% of their body weight or if there are other signs of significant toxicity. If all 3 mice must be sacrificed or die, the next 3 dose levels (e.g. 50, 35 and 12.5 mg/kg) are tested in a similar manner, whereas if only one or two dies, or needs to be sacrificed, the next 3 dose levels are between the highest shown safe dose thus far and the lowest lethal/toxic dose shown thus far. This process is repeated until a maximal tolerated dose (MTD) is found.

A compound of this disclosure, a compound that preferentially inhibits F₁F₀ ATP hydrolysis over F₁F₀ ATP synthesis, for example at least one compound of at least one of Formula (I), (II), (III), (IV), (V), (VII), (VIII), [X], or a pharmaceutically-acceptable salt, solvate, hydrate or prodrug thereof has the peculiarity that its MTD is higher if the animal is housed at 37° C. instead of normal room temperature (˜22° C.). The MTD and/or LD₅₀ and/or LD₃₀ and/or LD₁₀ and/or No-Observed-Adverse-Effect Level (NOAEL) of a compound(s) of this disclosure, optionally compound 7b, is investigated and recorded at both temperatures, optionally at interim temperature(s) also. This information is then useful for implementing other example embodiment(s) of this disclosure. The MTD (or other drug dose safety measure) to use, the value to use and apply in designing a study, depends on what temperature(s) the animal(s) is to be housed at in the study. This different MTD at different temperature aspect to a compound(s) of this disclosure is more pronounced the smaller the animal e.g. more pronounced/important for mice than rats.

Lifespan Extension Using a Compound(s) of this Disclosure

An example embodiment of this disclosure is to use a compound(s) of this disclosure, a compound that preferentially inhibits F₁F₀ ATP hydrolysis over F₁F₀ ATP synthesis, for example at least one compound of at least one of Formula (I), (II), (III), (IV), (V), (VII), (VIII), [X], or a pharmaceutically-acceptable salt, solvate, hydrate or prodrug thereof in an animal (e.g. mouse) lifespan study. Illustratively, compound 7b is used in a mouse lifespan study. For non-restrictive example, 300 six-week old female Mus Musculus C57BL/6 strain mice are sourced from a commercial vendor (e.g. Charles River Laboratories Inc., MA, USA). Alternatively more mice can be used to be able to statistically detect smaller percentage increases in lifespan. In an alternative embodiment male mice are used also, wherein a study with both sexes valuably permits gender difference(s) to be identified. However, males bring the additional complexity of fighting, which can lead to mice deaths (need more males than females because some males will be invariably lost to fighting). In other example embodiments another mouse strain(s) is used and/or genetically heterogeneous mice, which avoids genotype-specific effects on disease susceptibility. The mice are housed at 37° C., which is safe for mice (refer [30]), by setting the room/ambient temperature accordingly e.g. by placing their cage(s) (3-5 mice per cage) in a Plant-Growth/Veterinary or Animal Intensive Care (ICU) incubator(s) set at this temperature, wherein such incubators can be sourced from one or more of Precision Refrigerated Plant-Growth Incubators, Thermo Fisher Scientific, Darwin Chambers Inc., Powers Scientific Inc, Brinsea Products Ltd., Lyon Technologies Inc., or similar company, wherein some of these companies even make custom incubator designs. The electrical cost of this temperature maintenance is reduced by running the study in a hot geographical location/country, Singapore. Mice are kept on a 12 hour light/dark cycle, in 40-70% humidity, with corn cob bedding and have ad libitum sterilized/irradiated chow (illustratively AlN-93G standard diet or Purina 5LG6 or Purina 5001) and water. Preferably the mice are housed in a pathogen-free barrier environment (SPF conditions). One of the art knows how to successfully look after laboratory mice and there are well known guidelines and guides publically available. Mice are randomly allocated into two groups: 100 mice are in the drug treatment group, 200 mice are in the non-drug control group (twice more mice in control than drug group). Optionally a positive control (100 mice, no drug administered, calorie restricted diet) group is added. In some example embodiments the test drug is administered to the mice through drinking water/solution (in which case fluid intake of drug treatment and control groups is recorded). Indeed, there are compounds of this disclosure that are orally bioavailable e.g. 6b is 47% orally bioavailable in rats when administered in polyethyleneglycol:water:ethanol (1:1:1) solution [8]. Administering 6b as a salt, e.g. 6b HCl, increases its solubility, which is advantageous for oral administration via drinking solution. Alternatively, the drug (base and/or salt) is mixed in with previously irradiated (sterilized) chow, wherein the drug is 0.0001%, or 0.001%, or 0.01%, or 0.05% (recommended starting percentage for experimentation to find optimal percentage), or 1%, or 2%, or 3% or another percentage of chow weight, which is done by BioServ (Flemington, NJ, USA) or TestDiet Inc. (TestDiet, Richmond, IN, USA) or Dyets Inc. (Bethlehem, PA, USA) or a similar company/service, the drug content of chow is checked using HPLC, wherein this chow is produced every 2 months during the length of the study, and is (alongside untreated chow) stored refrigerated, is never permitted to exceed 40° C. and is kept away from light whenever possible to ensure drug stability (the light/dark cycle in the mouse facility is not altered). Preferably, water and chow are warmed to 37° C. before being accessible to the mice. To calculate how many mg/kg of drug any given mg/kg of drug in chow will deliver, a 30 g mouse consumes ˜5 g food/day [31] (⅙ of body weight, which is an approximate relation that can be applied to younger and lighter mice also), so illustratively, to deliver 40 mg/kg of drug to mice per day requires drug to constitute 240 mg/kg (0.024%) of chow. Optionally, further mice can be sourced for multiple drug treatment groups, all with the same number (100) of mice, which differ in the percentage weight of chow that is the test drug. So, that mice of the different drug treatment groups are administered a different drug dosage. Optionally, the test drug is microencapsulated e.g. by Southwest Research Institute (San Antonio, Texas) using a spinning disk atomization coating process with the enteric coating material Eudragit S100 (Rohm Pharma). This thermoplastic coating material increases the drug fraction that survives the chow preparation process. Because the coating material is water soluble only in non-acidic conditions, the encapsulated drug is released in the small intestine rather than in the stomach. It is prudent to verify that the compound retains activity after incorporation into mouse chow and that therapeutic blood levels of the drug can be achieved (blood drawn from tail vein). Method(s) to record the amount of a drug in blood is well known to those of the art e.g. using HPLC with ultraviolet detection [32] and/or LC-MS and/or LC-MS/MS. If eating the chow kills the mice, iteratively reduce the drug content of the chow until the mice can safely survive eating the chow. To observe if there is sufficient drug in chow to cause a physiological effect, house the mice at 22° C. and record the rectal temperature of the mice every 15 minutes and observe if their body temperature falls. If so, there is a working drug concentration in the chow. Optionally, increase the drug content of chow until the body temperature drop is sufficient to kill the mice in this 22° C. study. Then check that this drug content of chow doesn't kill the mice when they are housed at 37° C., if not, proceed with the study, if so, reduce the dose until finding the largest safe drug dose in chow at 37° C. Either use this largest safe dose or some fractional function (e.g. half e.g. 10% e.g. another percentage) of it. The recommended oral starting dose of 6b is 80 mg/kg≈0.05% weight of chow, wherein this recommendation is extended to compound 7b also. However, in other embodiments a different chow drug percentage is used and one of the art will be able to experiment with different chow drug percentages to explore the best positioning/compromise between drug safety and maximal drug effect, wherein preferably a Maximal Tolerated Dose (MTD) study, as described elsewhere herein, would have been performed prior to give further information to guide this assessment. Methods to derive a drug dose to be used in a drug trial, if the drug's MTD/LD₅₀ (drug dose that kills 50%)/LD₁₀ (drug dose that kills 10%) is known, are well known in the art. In an embodiment, the No-Observed-Adverse-Effect Level (NOAEL) is found and used, or some selected fraction (e.g. 50%, 10% or other) of it, wherein most optimally the NOAEL is found for the route (e.g. oral) and method (e.g. chow) of drug administration used in the lifespan study. MTD studies are typically single dose studies whereas this will be a long term study with the drug being administered frequently over a long period and this distinction needs to be considered. Pilot studies with small numbers of mice can be very instructive for setting the parameters of larger studies with many mice. In an alternative embodiment the drug is (e.g. daily) administered intravenously (e.g. at tail vein) through a catheter wherein control mice also have a catheter fitted and are administered vehicle at the same frequency as test mice are administered [drug+vehicle]. In other embodiments the drug is administered by some other route/method of administration. Food intake (important to record because calorie restriction extends life [33], so any difference in food intake between the drug treatment and control groups needs to be known; food intake of drug treated mice will be less than control mice because the drug renders their metabolism more efficient requiring less food and so they will choose to eat less food) and body weight are measured on a biweekly or bimonthly basis for the duration of the study. Healthspan assays (e.g. as set out in [34, 35, 36] and/or assaying homeostatic capacity and/or observing heart rate variability and/or rotarod assay(s) and/or grip assay and/or horizontal bar assay and/or GSSG/GSH ratio and/or NAD/NADH ratio determination and/or one or more of the healthspan/functional assays listed in another example embodiment of this disclosure and/or another assay of the art e.g. recording one or more of body coordination, memory, learning, movement, cognitive function) can be performed at regular intervals, especially as the mice get older. During the study, the day that each mouse dies is recorded and the study ends when all mice have died. Survival curves are plotted using the Kaplan-Meier method, which includes all available animals at each time point. Statistical analyses is performed using JMP IN (SAS, Cary, NC). The criteria for euthanasia is based on an independent assessment by a veterinarian, according to AAALAC guidelines and only cases, where the condition of the animal is considered incompatible with continued survival, are represented in the curves. Every animal found dead or euthanized is necropsied for pathology score. At study end, the mean, median and maximal lifespan is calculated separately for drug treated and control groups. Comparing the proportion of mice still alive in each group at each age when the pooled population reaches the 90% mortality point is also a useful measure. The data will show that 7b extends the lifespan of mice, especially if they are maintained at 37° C. This temperature dependence aspect can be shown by running the experiment again, or in parallel, wherein all the mice (drug treated and control) are kept at 22° C. rather than 37° C., wherein there is a lower drug dose(s) with the drug treated group(s) at this lower ambient temperature (because the tolerated drug dose is lower at lower ambient temperature), and wherein, with lower drug dose, the lifespan extension isn't as great. In an alternative embodiment, when mice are first sourced they are older e.g. older (e.g. old) mice are sourced from the National Institute on Aging Aged Rodent Colony or from the Jackson Laboratory (USA, has 19.5 months old mice available, roughly equivalent to a 50 year old human). This means that the experiment will take less time to run, because the mice will die sooner after being received. But the increase in lifespan observed will be less. Another way to shorten the duration of the study is to use mice that undergo accelerated aging [37, 38, 39, 40]e.g., without limitation, Senescence Accelerated Mouse-Prone 8 (SAMP8) mice (approximately half the lifespan of normal laboratory mice; commercially available from Harlan Laboratories, Bicester, UK; also available from the Society for Senescence-Accelerated Mouse (SAM) Research, Japan [http://www.samrc.jp], as are further senescence accelerated mouse strains) and/or BubR1^{H/H} progeroid mice [41] and/or XPD (e.g. XPD^{TTD} [42]) mutant mice (optionally carrying an additional mutation(s) in XPA and/or XPC) [43, 44](mice with a Trichothiodystrophy [TID] mutation in XPD, with XPC knocked out, have accelerated aging and only live 4-8 weeks) and/or XPC mutant mice [45.46](commercially available from The Jackson Laboratory, Stock No: 010563) and/or ERCC1 mutant mice (e.g. ERCC1^{-/-} [47]e.g. ERCC1^{Δ/-} mice carry a null mutation in one allele and a 7-amino acid truncation in the second allele, maximum lifespan is ˜6 months) 148, 49, 371 and/or Ku70 and/or Ku80 and/or Ku86 [50] and/or DNA-PK_{CS} mutant mice [51] and/or Caspase-2 mutant mice (commercially available from The Jackson Laboratory, Stock No: 007899) [52, 53] and/or ICE mice (Induced Changes in Epigenome) and/or some other accelerated aging mouse model of the art. Some of these accelerated aging mouse models, as are others not mentioned but that can be found by one of the art, are recognised models of human accelerated aging diseases. Alternatively, to shorten the study duration, one can use a smaller and shorter lived mammal species than mice (20 g) e.g. the Common Shrew (9 g) or the even smaller Etruscan shrew (1.8 g). An embodiment of this disclosure is to enter a compound(s) of this disclosure into the Major Mouse Testing Program (MMTP) and/or the National Institute on Aging's Interventions Testing Program (ITP) and/or use the same/similar/inspired testing protocol for a lifespan study using a compound(s) of this disclosure, or another lifespan study protocol in the literature or a lifespan study protocol conceived by someone of the art, optionally after their reading lifespan studies in the literature e.g., without limitation, [32, 54, 55, 56, 57, 58, 59]. A disclosure embodiment is to enter a compound(s) of this disclosure, or result(s) from using a compound(s) of this disclosure, into a mouse/rodent or other animal lifespan competition such as the Methuselah Mouse Prize (MPrize) and/or Palo Alto Longevity Prize and/or other/similar. A compound(s) of this disclosure extends lifespan by a direct anti-aging effect and also by an anti-cancer effect, reducing the incidence of, and by treating/ameliorating/preventing/combating cancer. Given the established link between age/aging and neurodegenerative disease, a compound of this disclosure, which slows aging as shown by this example, has utility as a therapeutic for neurodegenerative disease e.g. (without restriction) for Alzheimer's disease and/or dementia. Illustratively, Rapamycin extends mouse lifespan [32, 57] and exerts therapy in a mouse model of Alzheimer's disease 1601. An accelerated mouse model of aging, SAMP8, is concurrently a mouse model of Alzheimer's disease [61].

Instead of, or in addition to, using mouse death as an endpoint in this study, an aging/mortality biomarker(s) can be used, e.g. one or more listed in the database: http://mortalitypredictors.org/ [62] e.g. walking speed e.g. epigenetic/methylation/Horvath's clock. In this way, compound(s) effect on aging/mortality can be assayed before death. This is especially important for human studies with a compound(s) of this disclosure, or a pharmaceutically-acceptable salt, solvate, hydrate or prodrug thereof, optionally following on from mouse studies, given that humans have a long lifespan and so a surrogate endpoint (change in a biomarker(s) e.g. walking speed) to assess the change in aging/mortality is preferable to awaiting lifespan data.

Healthspan Assays Show that a Compound(s) of this Disclosure Slows Aging, Including Brain Aging, and Treats/Ameliorates/Prevents/Combats Neurodegenerative Disease(s), Including Alzheimer's Disease

APP/swePS1ΔE9 mice is a mouse model of Alzheimer's disease [63], available from The Jackson Laboratory (stock no: 004462). Senescence Accelerated Mouse-Prone 8 (SAMP8) mice display a phenotype of accelerated aging, with associated cognitive decline, and is a mouse model of aging driving Alzheimer's disease and/or dementia [61], available from Harlan Laboratories (Bicester, UK). In a disclosure embodiment APP/swePS1ΔE9 mice (or an alternative Alzheimer's disease mouse model {to illustrate and not restrict: from the Model-AD project and/or The Jackson Laboratory have a number of different Alzheimer's disease mouse models available [typically present learning deficit, from variable age, many including spatial learning deficit], or the PDAPP (also known as hAPP(J20) transgenic mouse model of Alzheimer's disease [60]}, or a mouse model of a different neurodegenerative disease e.g. a mouse model of Parkinson's disease, optionally sourced from The Jackson Laboratory) are used for the following study. In a different disclosure embodiment SAMP8 mice (or an alternative accelerated aging mouse model) are used in the following study. In a different disclosure embodiment normal mice are used in the following study. This study will now be described with SAMP8 mice. At all places that SAMP8 is referred to, in another embodiment, “APP/swePS1ΔE9” is substituted in its place. At all places that SAMP8 is referred to, in a further embodiment, “normal” is substituted in its place. Six-week old male SAMP8 mice are sourced and randomly assigned to the following groups: 200 SAMP8 mice are maintained on control chow (LabDiet 5015, TestDiet, Richmond, IN) and 100 SAMP8 mice are maintained on chow (LabDiet 5015) that contains a compound(s) of this disclosure: a compound that preferentially inhibits F₁F₀ ATP hydrolysis over F₁F₀ ATP synthesis, for example at least one compound of at least one of Formula (I), (II), (III), (IV), (V), (VII), (VIII), [X], or a pharmaceutically-acceptable salt, solvate, hydrate or prodrug thereof. Illustratively, compound 7b of this disclosure. How to prepare such chow has been disclosed for an earlier disclosure example. Food consumption and body weight are monitored during the study. In a further example embodiment there is more than one drug treated group, wherein these groups differ in drug dose given. Preferably all mice are housed at 37° C., as described for a prior disclosure example, and preferably the behavioural experiments are performed at 37° C. also, wherein preferably water in the water tests is also at 37° C. Mouse body weights are measured regularly. Behavioural tests are conducted every month. The study ends when all mice die. As the mice get older, drug treated SAMP8 mice start to outperform control SAMP8 mice in one or more of these tests, and/or a test variant(s), and/or a similar test(s), and/or another test(s) of mental/cognitive faculty(s) e.g. as found in the literature or as modified by someone of the art upon reading the literature: for the following test(s) the experimenter is blinded to which mice are drug treated and non-drug treated, preferably all tests are videotaped for parallel independent confirmatory analysis by another experimenter(s), wherein automatic computer software analysis is used where available/possible to aid analysis:

(1) Activity. The open field test is performed using MED Associates hardware and Activity Monitor software according to manufacturer's protocol (MED Associates Inc, St. Albans, VT, USA). Animals are individually placed into clear Plexiglas boxes (40.6×40.6×38.1 cm) surrounded by multiple bands of photo beams and optical sensors that measure horizontal and vertical activity. Mouse movement is detected and recorded for 30 minutes by breaks within the beam matrices. Old (e.g. 10 months) drug treated SAMP8 mice have a higher average velocity of movement, moving greater distance and a greater number of vertical movements than equally old SAMP8 control mice.

(2) Prudence. Elevated Plus maze. Young healthy mice have an aversion to open spaces. Mental decline is associated with dis-inhibition and greater comfort/time spent in open spaces. The elevated plus maze consists of four arms (two open without walls and two enclosed by 15.25 cm high walls) 30 cm long and 5 cm wide in the shape of a plus. A video camera mounted overhead on the ceiling linked to video tracking software (Noldus Etho Vision) is used to collect behavioural data. This software detects and records when mice enter the open or closed arms of the maze and the time spent in each. Mice are habituated to the maze for 1 minute before testing by placing them in the centre of the maze and blocking their entry to the arms. Dis-inhibition is measured by comparing time spent on open arms to time spent on closed arms over a 5 minute testing period. Old (e.g. 10 months) drug treated SAMP8 mice have less dis-inhibition than equally old control SAMP8 mice.

(3) Memory, object recognition. Young healthy mice spend more time exploring a novel object than a familiar one. Mice are tested in a standard home cage. Phase 1 (Habituation): Each mouse is placed into the apparatus (no objects present) for two 10 minute sessions separated by 1-4 hours to habituate to the testing environment. Phase 2 (Training): Two identical velcro-backed objects (object “A”) are attached into designated corners of the apparatus. The mouse is placed into the apparatus opposite to the objects and recorded by a camera for 10 minutes. Phase 3 (Test): One hour after training, the test phase begins. Only one of the objects is replaced with a new object (object “B”). The mouse is placed into the apparatus opposite to the objects and recorded for 5 minutes. The apparatus is wiped and objects cleaned with 70% alcohol to remove odours between mice. “Object recognition index” is calculated by dividing the amount of time spent with (touching with nose or nose pointing at object and within 0.5 cm of object) object B by the total time spent with objects A +B and multiplied by 100. Old (e.g. 10 months) drug treated SAMP8 mice have a greater recognition index than equally old control SAMP8 mice.

(4) Memory, learning, re-learning. Barnes maze: The maze consists of a flat circular surface (36″ diameter) with 20 equally spaced holes (2″ diameter) along the outer edge. One of the holes leads to a dark hide box while the other 19 lead to boxes that are too small to be entered. The latency to enter the hide box is recorded. The test is conducted in three phases. Phase 1 (Training): A hide box is placed under one of the holes. Animals are placed into an opaque cylinder in the centre of the maze for 30 seconds to promote spatial disorientation at the start of the test. After 30 seconds, the cylinder is removed and the animal explores the maze until it finds and enters the hide box. The number of incorrect entries (nose pokes and head deflections over any hole that did not have the hide box beneath it) is scored. If the mouse fails to enter the box within 3 minutes, it is gently led into the box. The animal then remains in the box for an additional 20 seconds before it is removed from the box and gently placed into the home cage. Training is repeated three times a day for four days. The location of the hide box remains the same during every trial but it is shifted between subjects to reduce the potential for unintended intra-maze cues. Phase 2 (Retention): This phase measures retention of spatial memory following a delay. After a two day break from training, each animal is re-tested for a one day, three-trial session using the same hide box location as before. Phase 3 (Reversal): This phase examines memory reversal. On the day following the retention phase, a new hide box location is established 180 degrees from the original location. The same method as before is used and trials are repeated three times a day over two consecutive days. Old (e.g. 10 months) drug treated SAMP8 mice find the hide box faster, better retain knowledge of where the hide box is and learn faster a new location of the hide box than equally old control SAMP8 mice.

(5) Spatial navigational memory. Two-day water maze. Mouse tracking is performed using SMART version 2.0 (Panlab). The water is painted milk-white with nontoxic paint. A platform which is visible during training on Day 1 is then submerged just under the water level during testing on Day 2 and mice use spatial cues on the wall around the pool to navigate to the platform during testing. During testing on Day 2, the time it takes each mouse to find the hidden platform is measured. Old (e.g. 10 months) drug treated SAMP8 mice find the hidden platform faster than equally old control SAMP8 mice.

(6) Morris Water Maze 1641. Mouse tracking is performed using SMART version 2.0 (Panlab). The water is painted milk-white with nontoxic paint. 4 trials per day for 5 consecutive days. For each trial, mice are placed in the pool at 1 of 4 start locations. The starting locations are separated by 90° and are termed south, west, north, and east. Mice start a trial once from each of the 4 possible start locations on each day. The goal platform is positioned 45 cm from the outside wall in the south quadrant of the maze for all groups. The latency to find and mount the hidden platform is measured. Swimming speeds are also recorded to assess drug-induced motor effects. If the mice fail to find the platform before 120 seconds expires, they are placed on the platform by the experimenter. Mean daily latency to find the goal platform is calculated for each mouse. On day 6, the platform is removed and time spent in the platform quadrant determined. The water tank is surrounded by opaque dark panels with geometric designs at approximately 30 cm from the edge of the pool, to serve as distal cues. Old (e.g. 10 months) drug treated SAMP8 mice swim faster and reach the hidden platform faster, and spend longer in the platform quadrant when it is removed, than equally old control SAMP8 mice.

(7) Fear conditioning, fear memory, associative learning. A mouse freezes if it remembers and associates that environment with an aversive stimulus. Mice are trained on Day 1 to associate their environment with an aversive stimulus (a foot shock). The amount of time spent freezing in response to the environment is measured on Day 2. Fear conditioning is performed in a conditioning chamber (Med Associates) equipped with a grid floor via which the foot shock can be administered. Each mouse is placed inside the conditioning chamber for 180 seconds. A foot shock (2 seconds, 0.4 mA) is delivered 148 seconds after placement in the chamber. Twenty-four hours later, context-dependent freezing is measured during 3 minutes. Time spent freezing is measured using Any-Maze™ software. To avoid any influence of foot shock exposure on further testing, this is the last test performed of the battery of tests and all other tests are carried out in tests rooms other than the fear conditioning test. Old (e.g. 10 months) drug treated SAMP8 mice spend more time freezing in response to the context associated with the aversive stimulus than equally old control SAMP8 mice.

(8) As measured by an assay(s) described in [65], old (10+ months) drug treated SAMP8 mice have less hearing and/or vision loss (have a lower hearing threshold and/or a greater visual contrast sensitivity) than old (10 months) control SAMP8 mice.

(9) As measured by an assay(s) described in [66], old (10+ months) drug treated SAMP8 mice have greater neuronal plasticity (e.g. greater hippocampal synaptic plasticity e.g. greater Long-Term Potentiation [LTP] with excitatory neurons), and less neuronal degeneration and reactive astrocytosis, than old (10 months) control SAMP8 mice.

(10) Social preference test (SPT). Assesses sociability and social novelty preference (i.e. social recognition memory). The apparatus consists of 3 chambers, a central chamber (length: 9 cm, width: 18 cm, depth: 20 cm) and two outer chambers (6 cm*18 cm*20 cm). The dividing walls are made of clear Plexiglas, with square passages, 4 cm high and 4 cm wide. One circular cage (i.e. mouse enclosure) is placed into each outer chamber. The mouse enclosures are 15 cm in height with a diameter of 7 cm and bars spaced 0.5 cm apart to allow nose contact between mice but prevent fighting. The chambers and enclosures are cleaned with 30% ethanol in-between trials (inter-trial interval of 5 minutes) and fresh corn cob bedding is added prior to each test trial. Test animals are isolated for an hour prior to the start of testing. During the habituation trial, two mice are placed individually in the central chamber and allowed to freely explore the apparatus and the two empty enclosures for 5 minutes. For the sociability test an unfamiliar adult male mouse is placed in one of the two enclosures (i.e. opponent chamber) in a quasi-randomised fashion. Then the test mouse is returned to the apparatus and allowed to explore all three chambers for 10 minutes. Finally, test animals are observed in a 10 minute social recognition test. For this, a second, unfamiliar mouse is placed in the previously empty chamber so that the test mouse has the choice to explore either the familiar mouse (from the previous trial) or the novel, unfamiliar mouse. AnyMaze™ tracking software is used to determine the time spent in the different chambers, number of entries and distance travelled by the test mice in each trial. Time spent sniffing the opponent is recorded manually (i.e. snout of test mouse within the enclosure containing the opponent mouse or <5 mm away from enclosure). Old (e.g. 10 months) drug treated SAMP8 mice spend more time with the novel individual, as compared to time spent with the familiar individual, than equally old control SAMP8 mice.

(11) Olfactory test (i.e. cookie test). Test mice are familiarised with a high carbohydrate food (Froot Loops: Kellogg Pty. Ltd., Strawberry Hills, Australia) in their home cages, 24 hours prior to the test. Consumption is observed by the experimenter to ensure the novel food is palatable for the mice. On test day, test mice are habituated for 5 minutes to a large opaque cage (47 cm*18 cm*13 cm) containing 2 cm deep bedding. The animal is removed from the cage thereafter, and one Froot Loop is buried randomly in the cage bedding. The animal is then returned to the cage and given 10 minutes to locate the buried food. The latency to find the Froot Loop is recorded. Old (e.g. 10 months) drug treated SAMP8 mice will find the Froot Loop faster than equally old control SAMP8 mice.

(12) In treadmill testing, old (e.g. 10 months) drug treated SAMP8 mice have a faster maximal running speed, and greater running endurance, than equally old control SAMP8 mice, whether trained or untrained at treadmill running.

(13) Old (e.g. 10 months) drug treated SAMP8 mice have one or more of better Blood Brain Barrier (BBB) homeostasis, less inflammation (e.g. in the brain), less gliosis, better vascular function (e.g. in the brain), less Amyloid beta (AP), less tau protein (and/or less hyperphosphorylation of tau protein), lower levels of Vascular Cell Adhesion Molecule 1 (VCAM-1, a protein associated with vascular endothelium inflammation), lower levels of endogenous immunoglobulin G (IgG, high levels observed in old mice as consequence of disrupted BBB permeability), less glial fibrillary acidic protein (GFAP) expression, increased brain Docosahexaenoic Acid [DHA](possibly because of less oxidation of DHA, DHA is the primary structural fatty acid in the human brain and has been linked to cognitive performance. Low plasma levels of DHA are associated with cognitive decline in elderly and Alzheimer's disease patients, higher DHA intake and plasma levels inversely correlate with Alzheimer's disease risk, DHA supplementation in aged animals enhances learning and memory [67]), increased brain glutamate levels (brain [glutamate] decrease with age [68] and low [glutamate] has been observed with Alzheimer's disease [69, 70]) and/or a lesser pro-oxidant status in the brain than equally old control SAMP8 mice. (14) Old (e.g. 10 months) drug treated SAMP8 mice have less aging (are more similar to young SAMP8 mice), at one or more of the cognitive/movement/anatomical/physiological/electrophysiological/cellular (e.g. number of senescent cells [71])/biochemical/neurochemical/protein/protein modification (e.g. carbamylation 1721)/oxidation e.g. [73, 74]/metabolite/metabolic/epigenetic/histone loss/histone modification/telomere length/gene expression/DNA/DNA modification (e.g. DNA methylation)/RNA levels, than equally old control SAMP8 mice, for example as reported using one or more of the assays described in [75, 76, 77, 78, 79, 80, 81, 82, 83] or some other aging assay(s) of the art e.g. as described in the literature e.g. using/leveraging an aging/mortality biomarker(s) reported in the database: http://mortalitypredictors.org/ [62].

(15) Transcriptional drift is an age-associated loss of coordination among groups of genes 184, 851. Aging causes genes within functional groups to change expression in opposing directions, which cause a transcriptome-wide loss in mRNA stoichiometry and loss of co-expression patterns in aging animals, as compared to young animals. Observing hippocampal gene expression data, old (e.g. 10 months) drug treated SAMP8 mice have less transcriptional drift than equally old control SAMP8 mice i.e. old (e.g. 10 months) drug treated SAMP8 mice have a transcriptome (e.g. hippocampal transcriptome) more similar to young SAMP8 mice than equally old control SAMP8 mice. Metabolomic/metabolic drift is an age-associated change in the relative/absolute amounts of metabolite(s) e.g. reduced [NAD*][146, 138], increased AMP/ATP etc. [87]. Old (e.g. 10 months) drug treated SAMP8 mice have a (e.g. plasma and/or brain [e.g. hippocampal]) metabolome more similar to young SAMP8 mice than equally old control SAMP8 mice i.e. old (e.g. 10 months) drug treated SAMP8 mice have less (e.g. plasma and/or brain [e.g. hippocampal]) metabolomic/metabolic drift than equally old control SAMP8 mice. Optionally metabolome analysis is performed using Precision Metabolomics™ (Metabolon Inc., Morrisville, NC, USA).

### Hypermetabolism

A disclosure embodiment is a method in which a subject takes or is administered an effective amount of a compound(s) of this disclosure, for example at least one compound of at least one of Formula (I), (II), (III), (IV), (V), (VII), (VIII), [X], and/or another compound that selectively inhibits F₁F₀ ATP hydrolysis, or a pharmaceutically-acceptable salt, solvate, hydrate or prodrug thereof, optionally in co-therapy with one or more of an anti-thyroid drug(s) (illustrating, without restriction, carbimazole, methimazole, propylthiouracil/PTU, potassium perchlorate), radioiodine, beta blocker(s) (illustrating, without restriction, propranolol, metoprolol), surgery (thyroidectomy), to treat/ameliorate/prevent/combat one or more of hypermetabolism, heat intolerance, thyroidal hypermetabolism, non-thyroidal hypermetabolism e.g. Luft's disease.

Compounds of this Disclosure are Anxiolytics, Hypotensives,

### Anticonvulsants, Antipsychotics, Antidepressants, Antiemetics, Analgesics/Painkillers, Sedatives, Tranquilizers, Hypnotics and Antihistamines

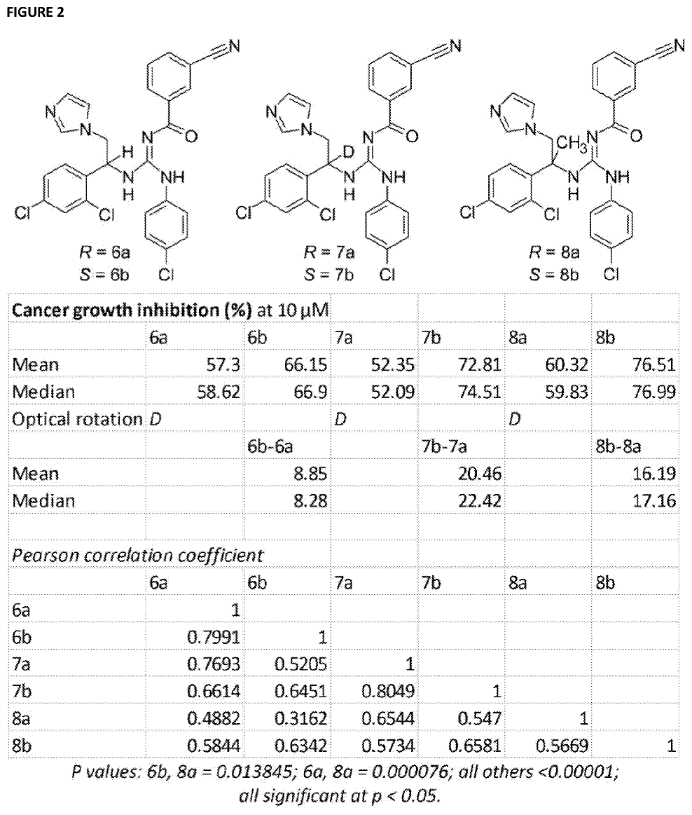

When mice were administered Compound 6b (its structure is in FIG. 2) they exhibited hypoactivity, the duration of which correlated with their drop in rectal temperature, which correlated with the administered dosage of 6b, wherein greater 6b dose caused greater rectal temperature drop and greater hypoactivity. Hypoactivity of mouse, after being administered compound 6b, was because 6b caused the mouse's body temperature to drop towards its ambient temperature (22° C.). When ambient equals optimal body temperature (37° C.), 6b cannot reduce body temperature and cannot cause hypoactivity. In some embodiments, the hypoactivity/sedation aspect to a compound(s) of this disclosure is utilized for therapy. The intersection between drug dose and ambient temperature dictates how much the body temperature falls and thence the depth of the sedation. Larger drug dose, and/or lower ambient temperature, causes deeper sedation (e.g. useful for inducing anaesthesia, pre-anaesthesia, post-anaesthesia, hypoesthesia, sedation, coma, tranquilization, behavioural submission, muscle relaxation and/or treating/ameliorating/preventing/combating insomnia, fatal insomnia, sleep onset latency, delayed sleep phase disorder, exploding head syndrome, parasomnia, sleep-maintenance insomnia, sleep disorder etc.; repeated/continuous administration, e.g. repeated i.v. injections or continuous i.v. infusion, of a compound(s) of this disclosure can sedate a subject for an extended period of time). Smaller drug dose, and/or higher ambient temperature, causes lighter sedation (e.g. useful for anti-anxiety, anti-depression, hyperactivity etc.). At ambient≥optimal body temperature (37° C.), no sedation occurs. Increasing sedation occurs with lower body temperature because action potential characteristics are temperature dependent. In some disclosure embodiments, deeper sedation is conveyed by a larger body temperature drop (body temperature<34° C.) and in other disclosure embodiments a slight calming sedation, optionally imperceptible to the subject, is conveyed by a smaller body temperature drop, optionally less than 1° C., optionally less than 0.5° C. To repeat, the intersection between dose and ambient temperature dictates the magnitude of body temperature drop, wherein this can be zero, even at high drug dose, when ambient temperature ≥37° C. So many diseases/disorders are because of too much/inappropriate/undesired signals/activity/electrical activity in the nervous system, wherein a compound(s) of this disclosure can decrease nervous system activity by a tunable degree (reduction amplitude set by intersection of drug dose with ambient temperature) and so can treat/ameliorate/prevent/combat an incredibly large number of diseases/disorders. Fundamental action equals broad applicability. Drug action against a fundamental physiological parameter (body temperature), which dictates a further fundamental physiological parameter (action potential characteristic(s): firing threshold/conduction velocity/firing frequency etc.), yields broad therapeutic application. For example, it raises the stimulus threshold for an epileptic seizure, which decreases the frequency of epileptic seizures in a subject. For example, it raises the stimulus threshold for ejaculation, therefore delaying ejaculation during sex, therefore assisting a subject with premature ejactulation. For example, it raises the threshold for pain perception and so any pain is reduced in magnitude. For example, in the brain, it raises the threshold for a “behaviour suggestion signal” to be selected for action, thence reducing the occurrence of unwanted behaviour (e.g. in Tourette's syndrome). Reduced temperature decreases action potential (AP) conduction velocity (Q₁₀=˜1.7, so AP velocity is ˜10% less at 35 than 37° C. 188, 891), decreases AP frequency (30% decrease in spike rate with 2° C. decrease in temperature [90]), increases AP firing threshold (AP firing threshold vs. temperature is U shaped because threshold increases as move away from an optimum temperature [91, 92]) and decreases neural circuit activity in vivo [93].

Because a compound(s) of this disclosure can cause sedation (when ambient<optimal body temperature {37° C.}) and slow aging, this juxtaposition makes a compound(s) of this disclosure useful for inducing hibernation/artificial hibernation/torpor/synthetic torpor/suspended animation, optionally used on a long journey, optionally during spaceflight, optionally on a journey to Mars (projected duration with present technology is ˜18 months transit time for round trip). Moreover because the compound(s) reduces food, power (e.g. reduced lighting/heating), living space and O₂ requirements in a subject, this makes the spacecraft load lighter, and the induced lower respiration rate means slower/shallower breathing, less O₂ in the body, which reduces the damaging effect of ionizing radiation (significant in space), moreover permitting a lower O₂ concentration outside the body, which reduces ionizing radiation damage to the outside of the body, and the smaller living space afforded by the sedation permits greater radiation shielding per unit living space and the induced hypometabolism reduces the rate of muscle and bone atrophy (reduces spaceflight osteopenia) and other negative health effects (e.g. sleep disturbance) of microgravity, and the sedation side-steps the anticipated worrying problem of interpersonal friction(s) during long confined spaceflight (Cosmonaut Valery Ryumin's autobiography: “If you want to instigate the art of manslaughter just shut two men up in a eighteen by twenty-foot cabin for a month. Human nature won't stand it.”). Optionally the drug can be administered by continuous intravenous infusion, wherein optionally respiratory substrates, nutrients, fluids etc. can be administered similarly (e.g. using parenteral nutrition). If there is a job/emergency that needs to be attended to in the spacecraft (and/or the subject is to eat/wash/administrate themselves etc.) the hibernation is paused by raising the ambient temperature of the subject to 37° C. Afterwards, assuming the subject still has sufficient compound(s) in their system, the hibernation can be induced again by lowering the ambient temperature. During spaceflight, the use of a compound(s) of this disclosure combats many of the problems identified for space exploration/travel by NASA Report No. IG-16-003 (“NASA's efforts to manage health and human performance risks for space exploration”, Oct. 29, 2015, audit conducted by Office of Inspector General).

An aspect of this disclosure is to use a compound(s) of this disclosure to sedate (or to help sedate) a subject undergoing treatment (e.g. surgery, e.g. surgery to remove a tumour), and/or a course of treatment, for a pathology/disease/disorder/dysfunction/unwanted characteristic(s) of the subject. For non-limiting example, for a subject undergoing drug (e.g. opoid) withdrawal, wherein a compound(s) of this disclosure is used to sedate the subject during their drug withdrawal phase so they don't suffer the, typically horrific (why many drug users can't get off drugs), withdrawal symptoms such as pain, nausea, craving etc., which are worst in the first few days of withdrawal, a common time of drug relapse.

Because a compound(s) of this disclosure can cause sedation (when ambient<optimal body temperature {37° C.}), slow aging and exert anti-cancer activity, these attributes make a compound(s) of this disclosure useful for a subject undergoing anti-cancer treatment, optionally during a hospital stay, wherein more than the time the subject loses sedated is returned to them by a longer [life/health] span. When the cancer patient has a visitor the sedation can be paused by raising the ambient temperature to 37° C. (e.g. by transferring the patient's bed trolly into a visitors area/room maintained at this temperature). If a light sedation (small body temperature drop, cancer patient remains conscious but calmer, cancer patient can go about their normal life) is instead chosen for a cancer subject, there is a useful juxtaposition in the compound(s) anti-cancer and anxiolytic and/or antidepressant effects, because many cancer patients are anxious/depressed, and there is benefit to the compound's analgesic and/or antiemetic effects also, if radio/chemo-therapy is used in co-therapy, because radio/chemo-therapy typically causes cancer patients pain and nausea/vomiting, often extreme. Optionally a compound(s) of this disclosure is taken before the subject wishes to sleep, for example at night, and so any perceptible sedation, should it occur with the dose taken at that ambient temperature, is then virtuous rather than limiting to normal life.

Co-Therapy with an Uncoupler(s)

An embodiment is to administrate an (preferably therapeutically effective) amount of at least one compound(s) that inhibits F₁F₀ ATP hydrolysis (e.g. at least one compound of at least one of Formula (I), (II), (III), (IV), (V), (VII), (VIII), [X]), optionally with an (preferably therapeutically effective) amount of the same or a different compound(s) that uncouples the proton motive force (an uncoupler), for use in a method of treatment of the human or animal body by therapy, wherein optionally the F₁F₀ ATP hydrolysis inhibitor(s) and uncoupler(s) are in a single pharmaceutical composition and/or are packaged, and/or distributed, and/or sold together, optionally for the treatment/amelioration/prevention/combat of cancer and/or a disease/disorder partially/completely driven by, or made worse, by activated macrophages (or similar activated cell type e.g. pancreatic islet macrophages/Langerhans cells/dendritic cells/monocytes/histiocytes/Hofbauer cells/Kupffer cells/phagocytes/microglia/epithelioid cells/osteoclasts/macrophage like cells/cells of the mononuclear phagocyte system, and/or any cell type(s) of the innate immune system and/or of the monocyte lineage, especially inducible nitric oxide synthase (iNOS) and/or iNOS2 expressing and/or NO producing cells {e.g. monocyte-derived inflammatory dendritic cells}; activated macrophages, unlike resting macrophages, singly use and thence completely rely upon ATP synthase in its reverse mode, hydrolysing ATP, to maintain Ψ_{IM} [94]) in a subject(s),
optionally to treat/ameliorate/prevent/combat HIV infection/transmission/drug resistance, optionally (optionally in the same pharmaceutical composition) with an effective amount of a compound(s), protein(s), antibody(s), pathogen(s) or pathogen component(s) that activates macrophages (isn't absolutely necessary because HIV activates macrophages itself [95.96], which drives the chronic inflammation pathology component to HIV infection; HIV stimulates nitric oxide production by human macrophages [97, 98, 99, 100]), optionally with a compound(s), protein(s), antibody(s), pathogen(s) or pathogen component(s) that polarises macrophages towards M1 type (examples, without restriction, include Granulocyte-macrophage colony-stimulating factor [GM-CSF]and/or a pharmaceutical version/analog(s)/biosimilar of GM-CSF such as, without limitation, one or more of sargramostim, molgramostim, regramostim, filgrastim, pegfilgrastim), optionally in co-therapy (optionally in same pharmaceutical composition) with, or after, Anti-Retroviral Therapy (ART), combination Anti-Retroviral Therapy (cART, Highly Active Anti Retroviral Therapy, HAART), optionally in co-therapy (optionally in same pharmaceutical composition) with L-arginine (substrate for inducible Nitric Oxide Synthase, iNOS, optionally wherein food(s) rich in this amino acid is eaten), optionally for pre- and/or post-exposure prophylaxis (PEP) e.g. after needlestick injury and/or sex with an HIV infected person(s), e.g. to reduce the probability of mother to baby HIV transmission during pregnancy/birth/breast feeding e.g. given to a subject without HIV to decrease the risk they will acquire HIV, optionally wherein this subject is at sizeable risk of acquiring HIV, wherein this pre-exposure prophylaxis can reduce the spread of HIV within a population (for example in sub-Saharan Africa). Even after prolonged cART, which drives plasma HIV down to undetectable levels, HIV-1 DNA and RNA is detectable in macrophages: they are an HIV reservoir that remains extant, even during cART, and that the virus can spread from during any interruption or termination of cART [101]. Moreover, HIV virus recombines and mutates in macrophages [102], which is a drive to HIV drug resistance. Thence the vital importance of the methods and compounds herein. By denying HIV a reservoir in which to hide (and mutate, developing drug resistance) from ART/cART/HAART therapy, a compound(s) of this disclosure, or a pharmaceutically-acceptable salt, solvate, hydrate or prodrug thereof, decreases the amount of HIV virus in the body, increasing the chance of HIV viral elimination from the subject, decreasing the risk the subject can transmit the HIV virus to another subject, decreasing HIV associated symptoms/pathology, decreasing the chance of HIV developing drug resistance to one or more drugs used in ART/cART/HAART therapy, improving clinical outcome. Notably, a compound(s) of this disclosure, or a pharmaceutically-acceptable salt, solvate, hydrate or prodrug thereof, treats/ameliorates/prevents/combats HIV-associated chronic inflammation and/or HIV peripheral neuropathy, wherein the latter is caused by infiltration of HIV infected monocytes/macrophages to the dorsal root ganglia (DRG) causing neuronal loss and formation of Nageotte nodules.

In some embodiments, the activity (e.g. anti-cancer and/or anti-HIV activity, incidentally wherein both these activities are pertinent for a subject with an AIDS defining or HIV-associated cancer) of the uncoupler(s) and F₁F₀ ATP hydrolysis inhibitor(s) synergize (potentiate).

### Slow Release Formulations

A disclosure embodiment is to administer to a subject a therapeutic amount of at least one compound of this disclosure, for example at least one compound of at least one of Formula (I), (II), (III), (IV), (V), (VII), (VIII), [X], in a formulation/dosage selected from modified release, extended release, long acting release, sustained release, prolonged release, controlled release, slow release or similar, as clear to someone of the art, for use in a method of treatment of the human or animal body by therapy. Such a formulation exposes the subject body to the compound(s) over a longer period of time than if the compound was applied alone. This is useful because it delivers good area under the curve for the compound, which for example exerts anti-cancer activity in the subject, without an abrupt large body temperature drop. Any body temperature drop is less in amplitude, more in duration, which is safer.

### Temperature Controlled Release

A disclosure embodiment is a temperature-sensitive pharmaceutical composition/vehicle that only releases a compound(s) of this disclosure, for example at least one compound of at least one of Formula (I), (II), (III), (IV), (V), (VII), (VIII), [X], and/or other F₁F₀ ATP hydrolysis inhibitor(s), when the body is at normal body temperature or higher. The latter is reached if the subject has a fever for example. Many cancers cause fever. Such a temperature-sensitive delivery composition/vehicle, releasing drug(s) at normal body temperature (37° C.) for example, can effect a safety feedback loop because as F₁F₀ ATP hydrolysis inhibitor is released, body temperature falls, thence less drug is released, body temperature can thence recover, further compound is released, and this loop iterates, implementing extended release and minimising the perturbation to body temperature from optimal. For non-limiting example, a F₁F₀ ATP hydrolysis inhibitor(s) is loaded into a structure incorporating biocompatible thermo-sensitive polymer which undergoes a volume change (e.g. shrinks) at a temperature exceeding its phase/volume transition temperature, releasing the compound. This volumetric change is reversible. Should the temperature subsequently fall below the phase/volume transition temperature, the volume change reverses (e.g. structure expands) and compound release doesn't occur [103]. In some embodiments the phase/volume transition temperature is tuned to be at normal body temperature, in other embodiments, at a pathologically elevated body temperature(s). Biocompatible thermosensitive polymers can be used to prepare temperature-responsive hydrogels/nanogels and thence nanoparticles, optionally with polysaccharides to modulate the drug encapsulation and release efficiency, which have a phase transition temperature, above which they release the “cargo” compound(s). Transition temperature can be readily tuned by the copolymerization conditions and by varying the content of repeating units in the copolymer. Non-limiting options for making temperature sensitive vehicles for compounds of this disclosure include thermosensitive hydrogels/nanogels, temperature sensitive liposomes [104-106](these have been used in clinical trials e.g. ThermoDox), thermosensitive micelles, polymeric micelles, core shell structures, core-shell microgel particles, thermoresponsive composite films, smart three dimensionally ordered porous materials, thermosensitive microcontainers, nanoscale drug delivery vehicles.

Some Methods of this Disclosure

Use of at least one compound according to Formula [X](optionally at least one compound of at least one of Formula (I), (II), (III), (IV), (V), (VI), (VII), (VIII); and/or a pharmaceutically-acceptable salt, solvate, hydrate or prodrug thereof, and/or a pharmaceutical composition thereof), and/or a selective/preferential F₁F₀ ATP hydrolysis inhibitor compound(s)/composition(s) (that preferably inhibits F₁F₀ ATP synthesis less or, more preferably, not at all), wherein an effective amount of compound(s)/composition(s) is administered to the subject topically/locally, wherein the reduction in metabolic heat generation at the administered body region(s) is substituted by heat transfer from other regions (e.g. by blood flow) of the subject's body, wherein this method delivers compound(s)/composition(s) conferred treatment to the administered body region(s) whilst the body temperature reduction side-effect of the compound(s)/composition(s) is mitigated/beaten, optionally wherein the administered body region(s) is one or more of the eye (or part(s) thereof), and/or ear (or part(s) thereof), and/or Central Nervous System/brain (or part(s)/cell type(s) thereof e.g. some or all dopamine neurons in the substantia nigra),
and/or skin (or part(s) thereof), and/or face (or part(s) thereof), and/or hair (or part(s) thereof), and/or a tumor/cancer (or part(s) thereof),
and/or wherein an effective amount of compound(s)/composition(s) is administered to the subject systemically,
and/or optionally the subject is monitored/observed/watched (optionally by a healthcare worker such as doctor, nurse, paramedic, EMT, vet, pharmacist, optionally by a research worker such as scientist, laboratory technician) after compound(s)/composition(s) administration for any adverse sign(s)/symptom(s)/abnormality,
and/or optionally the subject is located in an ambient temperature exceeding 30° C. and/or optionally the subject wears one or more items of clothing and/or optionally wherein the subject's body temperature is monitored (continuously or at regular/irregular time intervals),
and/or optionally wherein the greater the ambient temperature the subject is in, the greater the dose administered and/or optionally wherein the subject being in a higher ambient temperature can enable a higher dose to be administered,
and/or optionally wherein the ambient temperature to the subject, and/or their bodily insulation, is increased before they are administered with the compound(s)/composition(s),
and/or optionally wherein the subject moves to a different geography/space with a higher ambient temperature for compound(s)/composition(s) administration,
and/or optionally wherein a daily dose is spread over multiple doses per day so that any compound(s)/composition(s) driven body temperature drop in the subject is lowered in amplitude and lengthened in duration (which is safer),
and/or optionally wherein the subject stays/is inside a building, optionally which is heated (so hotter inside the building than outside), optionally their home, optionally their workplace, optionally a hospital, whilst they have an effective amount of compound(s)/composition(s) in their system,
and/or or optionally wherein the subject stays/is inside a vehicle, optionally which is heated (so hotter inside the vehicle than outside), whilst they have an effective amount of compound(s)/composition(s) in their system,
and/or optionally wherein the compound(s)/composition(s) is administered to the subject (e.g. once per day prior to sleep, e.g. once per day orally prior to sleep) at such time that the Cₘₐₓ and/or much/most/all of the “Area Under the Curve (AUC)” occur whilst the subject is asleep/resting/relaxing/trying to sleep/indoors/inside/working indoors and/or in a single place/area/home/house/building/complex/workplace/office/room/bedroom/confinement/vehicle for more than 3 hours (or for more than 5 hours, or for more than 8 hours, or for more than 10 hours, or for more than 12 hours), e.g. at night, and/or optionally wherein the subject sleeps/rests/relaxes/works in a higher ambient temperature, e.g. because of shelter and/or heating and/or insulation, than the outside/climate temperature in that location at that time,
and/or optionally wherein the temperature/climate/season/weather/weather forecast in that location at that time dictate if the compound(s)/composition(s) is, or is not, administered to the subject and at what dose, and/or optionally wherein the subject is administered (and/or self-administers) the compound(s)/composition(s) shortly before they sleep, preferably wherein they are sheltered (e.g. inside instead of outside) and/or insulated (e.g. by bedding(s)/blanket(s), and/or clothing, and the like) whilst they sleep, optionally in a heated room/building/confinement that is set to a higher (safe) temperature than outside it.
and/or optionally wherein the subject is administered with compound(s)/composition(s) in a workplace of healthcare professionals such as a hospital,
and/or optionally the subject is monitored, for example by a healthcare/research professional(s) and/or machine substitute(s), for sign(s) of reduction in body temperature and/or the subject is located at an ambient temperature that maintains their body temperature within safe limits whilst they have an effective amount of compound(s)/composition(s) in their system and/or whilst the subject has an effective amount of compound(s)/composition(s) in their system the subject wears (and/or is covered by) insulating material(s), e.g. clothing/clothes (and/or bedding/blanket(s)), and/or is in a heated/insulated confinement/building/room/space (e.g. for conferring heat/hyperthermia therapy e.g. as used for cancer therapy) and/or hot climate, optionally exceeding one or more of 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C. optionally at or around 37° C., wherein a higher (e.g. in the thirties/forties ° C.), but safe, ambient temperature (and/or greater bodily insulation, for example by clothing/clothes and/or bedding/blanket(s)) can permit a greater compound(s)/composition(s) dose(s) to be safely administered, wherein a preferred ambient temperature is the thermoneutral temperature for the subject with the amount of bodily insulation they have, e.g. the amount of clothing they are wearing, if any, and the amount of the compound(s)/composition(s) in their system; and/or optionally wherein the subject's ambient temperature is measured, and/or inferred/estimated from information sourced (e.g. local weather report/prediction), before the subject is administered with compound(s)/composition(s), optionally wherein this data is used to select, or as one factor of multiple in selecting, the compound(s)/composition(s) dose administered to the subject;
and/or optionally wherein the subject's body temperature is measured whilst they have an amount of compound(s)/composition(s) in their system;
and/or optionally wherein the administered compound(s)/composition(s) dose doesn't reduce the subject's body temperature;
and/or optionally wherein the administered compound(s)/composition(s) dose doesn't reduce the subject's body temperature below their normal/typical body temperature;
and/or optionally wherein the administered compound(s)/composition(s) dose doesn't reduce the subject's body temperature lower than normal body temperature;
and/or optionally wherein administration of the compound(s)/composition(s) dose doesn't reduce the subject's body temperature at all or not more than 0.001 or 0.01 or 0.1 or 0.5 or 1 or 2 or 3° C. lower than the subject's body temperature before the administration of said compound(s)/composition(s); and/or optionally wherein administration(s) of the compound(s)/composition(s) dose doesn't reduce the subject's body temperature at all or not more than 0.001 or 0.01 or 0.1 or 0.5 or 1 or 2 or 3° C. lower than the subject's body temperature before the administration(s) of said compound(s)/composition(s), optionally wherein the subject's body temperature is measured one or more times within 1, and/or 5, and/or 10, and/or 30 minutes, and/or 1 hour, and/or 3 hours, and/or 6 hours, and/or 24 hours after the administration(s) of said compound(s)/composition(s);
and/or optionally wherein the administered compound(s)/composition(s) dose is the highest dose, or a fraction thereof, that doesn't, or that apparently doesn't, reduce the subject's body temperature at all or, if it does reduce body temperature, it only does so by a small amount (e.g. less than 1° C., or less than 0.1° C. or less than 0.01° C.);
and/or optionally wherein a different compound(s)/composition(s) dose is administered to a subject if they are in a different ambient temperature;
and/or optionally wherein a different compound(s)/composition(s) dose is administered to a subject if they are in a different ambient temperature and the ambient temperature is less than 37° C.; and/or optionally wherein a higher compound(s)/composition(s) dose is administered to a subject if they are in a higher ambient temperature;
and/or optionally an experiment(s) is performed in which different subjects (optionally rodents/mice) are kept at different temperatures (or within different temperature ranges) whilst having an amount of the compound(s)/composition(s) in their system;
and/or optionally a method comprising the following steps is conducted:
   (a) compound(s)/composition(s) is administered to the subject,
   (b) the subject's body temperature is measured sometime after (optionally recorded in vitro/ex vivo by recording the temperature/intensity of a sample(s)/electromagnetic radiation from the subject, optionally recorded non-invasively e.g. via thermal imaging),
      (c1) if the subject's body temperature is higher than, or at, normal body temperature (e.g. ˜37° C. for a human) or is reduced, but is still within desired/acceptable limits, further compound(s)/composition(s) is administered immediately/later at higher or same dose (especially/preferably selecting “same dose” if prior iteration(s) of this loop, comprising steps (a-c), have shown a higher than this present dose to produce unacceptably low body temperature(s)), wherein this compound(s)/composition(s) administration can be a step (a) for another iteration of steps (a-c),
      (c2) but if instead the subject's body temperature is lower than the desired/acceptable limit/range, optionally which can be the normal non-pathological body temperature (or range thereof) of a subject of this species, by contrast the subject is immediately/later administered no or less (e.g. 0.05%/10%/25%/50%/5%/90% or other % of immediately prior mg/kg dose, optionally wherein the % is tailored to the amount of body temperature reduction, whereby a smaller % is administered when the body temperature reduction is greater) compound(s)/composition(s) or the subject is administered with same/no/lower/higher compound(s)/composition(s) dose and the subject's ambient temperature and/or bodily insulation is increased, wherein if there is compound(s)/composition(s) administration it can be a step (a) for another iteration of steps (a-c),
   (d) wherein this system (steps a-c) can be iterated for a desired/selected/arbitrary number of iterations, optionally only stopping iterating once the desired or an acceptable level of disease/disorder therapy/treatment/prevention has occurred,
   (e) optionally wherein there is a preceding step, which is only executed once as the very first step, and which isn't included in subsequent iterations, wherein the subject's ambient temperature (or range thereof) and/or insulation is increased before compound(s)/composition(s) administration, optionally—in an alternative schema—the only steps in this schema are this present preceding step and step (a), wherein steps (b-c) are not included,
   (f) optionally wherein there is a preceding step to the aforementioned preceding step, or alternatively wherein this preceding step replaces the aforementioned preceding step, wherein the subject's body temperature is measured (optionally recorded in vitro/ex vivo by recording the temperature/intensity of a sample(s)/electromagnetic radiation from the subject, optionally recorded non-invasively e.g. via thermal imaging), optionally wherein if the subject's body temperature is less than normal, or alternatively not higher than normal, then the subsequent steps are not implemented,
   (g) optionally wherein different iterations of this system and/or step(s) thereof can be performed when the subject is in different ambient temperatures (or ranges thereof) and/or has different amounts of bodily insulation,
   (h) optionally wherein this system is started with a low mg/kg compound(s)/composition(s) dose and/or with a compound(s)/composition(s) dose lower than that shown to reduce body temperature to an undesirable/unacceptable degree at that (or similar) ambient temperature in another subject(s) of the same species, and more preferably of the same gender and of similar mass, optionally of similar age,
   (i) in some embodiments the subject is a human and in alternative embodiments the subject is a non-human species, preferably a mammal; and/or optionally wherein one or more of the following (Roman numeral points) applies when the subject has an effective amount of the compound(s)/composition(s) in their system:
   (I) the subject is sheltered and/or heated and/or insulated and/or clothed;
   (II) the subject is in a room/building/vehicle/shelter which has a higher ambient temperature than the ambient temperature outside;
   (III) the subject is in a heated and/or insulated area/confinement/room/building/vehicle/shelter;
   (IV) the subject wears one or more items of clothing;
   (V) the subject wears one or more items of clothing in a heated and/or insulated area/confinement/room/building/vehicle/shelter;
   (VI) the ambient/air temperature experienced by the subject is hotter than the climatic/outside/apparent air temperature in their geography at the time;
   (VII) the ambient/air temperature experienced by the subject, underneath their worn clothing/clothes, is hotter than the climatic/outside/apparent air temperature in their geography at the time;
   (VIII) the ambient/air temperature experienced by the subject is hotter than the climatic/outside/apparent air temperature in their geography at the time because of one or more of the subject being sheltered, heated (e.g. by the heating system of a room/building/vehicle that the subject is in), in a confinement with elevated humidity (e.g. wherein the subject is in a sauna or similar), the subject wears one or more items of clothing, the subject is covered by one or more insulating materials (e.g. blanket(s)/sheet(s));
   (IX) the subject is sheltered from one of more of the weather elements, including wind and/or rain/snow, by a building/vehicle/shelter and/or one or more items of clothing;
   (X) liquid (e.g. water) that the subject drinks is heated and is hotter than ambient temperature when it is drunk by the subject;
   (XI) food that the subject eats is cooked/heated and is hotter than ambient temperature when it is eaten by the subject;
   (XII) the subject is in a room/budling/complex/tunnel system underground in a higher ambient temperature than the ambient temperature overground at the same latitude and longitude;
   (XIII) the subject is in an underground burrow/room/building/complex/tunnel system, preferably which is situated in a tropical/equatorial climate region;
   (XIV) the subject is in an underground burrow/room/building/complex/tunnel system, situated in a tropical/equatorial climate region (e.g. Kenya), which ensures it is consistently warm, buffered from weather fluctuations (e.g. tropical rain storms) overground;
   (XV) the subject is in an underground burrow/room/building/complex/tunnel system with rooms and/or living/working/recreation/sleeping areas/quarters at different depths underground, which can thence inherently have different temperatures, and wherein the subject can select their ambient temperature by selecting their depth;
   (XVI) the subject's body temperature reduces/is reduced;
   (XVII) the subject's body temperature is not reduced, or isn't reduced by as large an amplitude, because of one or more of the above Roman numeral points applies/is implemented; alternatively wherein the compound(s)/composition(s) is administered to the subject to deliberately reduce their body temperature, optionally to a desired/specified/controlled temperature (or range thereof), wherein the amplitude of hypothermia conferred in the subject by the compound(s)/composition(s) administration is controlled by setting the ambient temperature, wherein a sufficient amount of administered compound(s)/composition(s) reduces subject body temperature to slightly higher than their ambient temperature, such that hypothermic amplitude is controlled by controlling ambient temperature; alternatively wherein the compound(s)/composition(s) is administered to the subject to deliberately reduce their body temperature, optionally to a desired/specified/controlled body temperature (or range thereof), wherein the amplitude of hypothermia conferred in the subject by the compound(s)/composition(s) administration is set by setting the quantity of compound(s)/composition(s) administered and/or setting the ambient temperature, wherein a larger dose can lower the subject's body temperature to nearer/near (but always above, if the subject is alive) the ambient temperature and a smaller dose can lower it by a fraction thereof;
alternatively wherein the compound(s)/composition(s) is administered to the subject to deliberately reduce their body temperature to a desired/specified/controlled temperature (or range thereof), wherein a sufficient amount of administered compound(s)/composition(s) reduces subject body temperature to a temperature that it cannot fall below because the subject is heated by incident electromagnetic radiation (optionally the intensity of which is controlled by servocontrol, with the set point set at the desired hypothermic body temperature, optionally from a radiation heater), which “catches” and offsets the hypothermic drive of an administered compound(s)/composition(s), at a desired/specified hypothermic body temperature (or range thereof);
and/or optionally wherein the administered compound(s)/composition(s) dose reduces an abnormally/pathologically elevated body temperature;
and/or optionally wherein the administered compound(s)/composition(s) dose reduces an abnormally/pathologically elevated body temperature (or range thereof) to normal body temperature (or range thereof);
and/or optionally wherein the administered compound(s)/composition(s) dose is that which reduces body temperature by the desired amount;
and/or optionally a method comprising the following steps is conducted:
   (α) compound(s)/composition(s) is administered to the subject,
   (β) the subject's body temperature is measured sometime after (optionally recorded in vitro/ex vivo by recording the temperature/intensity of a sample(s)/electromagnetic radiation from the subject, optionally recorded non-invasively e.g. via thermal imaging),
      (γ1) if the subject's body temperature is not at, or not lower, than normal body temperature (e.g. ˜37° C. for a human), or is not low enough to be within a desired range of lower body temperature, compound(s)/composition(s) is administered immediately/later at higher (e.g. 110%/150%/200% or other %, that is >100%, of immediately prior mg/kg dose) or same dose (especially/preferably selecting “same dose” if prior iterations of this loop have shown that this dose can reduce body temperature to the desired value/range of body temperature) or the subject is administered with same/no/higher/lower compound(s)/composition(s) dose and the subject's ambient temperature and/or bodily insulation is decreased, wherein if there is compound(s)/composition(s) administration it can be a step (α) for another iteration of steps (α-γ),
      (γ2) but if instead the subject's body temperature is within the desired range of body temperature, by contrast the subject is immediately/later administered same, no or less compound(s)/composition(s), wherein if there is compound(s)/composition(s) administration it can be a step (a) for another iteration of steps (α-γ),
      (γ3) but if instead the subject's body temperature is reduced, and it is actually lower than the desired range of reduced body temperature, by contrast the subject is immediately/later administered no or lower (e.g. 0.05%/10%/25%/50%/5%/90% or other % of immediately prior mg/kg dose, optionally wherein the % is tailored to the amount of excess unwanted body temperature reduction, whereby a smaller % is administered when the excess unwanted body temperature reduction is greater) compound(s)/composition(s) dose or the subject is administered same/no/lower/higher compound(s)/composition(s) dose and the subject's ambient temperature and/or bodily insulation is increased, wherein increasing the subject's ambient temperature to be just below the desired body temperature is particularly effective for correcting this overshoot error, wherein if there is compound(s)/composition(s) administration it can be a step (a) for another iteration of steps (α-γ),
   (δ) wherein steps (α-γ) can be iterated for a desired/selected/arbitrary number of iterations, optionally only stopping iterating once the desired or an acceptable level of disease/disorder therapy/treatment/prevention/surgery has occurred,
   (ε) optionally wherein there is a preceding step, which is only executed once as the very first step, and which isn't included in subsequent iterations, optionally wherein the subject's insulation is decreased before compound(s)/composition(s) administration, critically wherein the ambient temperature (or range thereof) is set to be below the desired reduced body temperature (or range thereof) and optionally wherein the ambient temperature is set to be 0.1-3° C. below the desired reduced body temperature or optionally further below, optionally—in an alternative schema—the only steps in this schema are this present preceding step and step (α), wherein steps (β-γ) are not included,
   (ζ) optionally wherein there is a preceding step to the aforementioned preceding step, or alternatively wherein this preceding step replaces the aforementioned preceding step, wherein the subject's body temperature is measured (optionally recorded in vitro/ex vivo by recording the temperature/intensity of a sample(s)/electromagnetic radiation from the subject, optionally recorded non-invasively e.g. via thermal imaging),
   (η) optionally wherein different iterations of this system and/or step(s) thereof can be performed when the subject is in different ambient temperatures (or ranges thereof) and/or has different amounts of bodily insulation,
   (η) optionally wherein the subject is also administered with a drug(s), preferably (but not restrictively) FDA/EMA licensed, to prevent/reduce/treat shivering (e.g. (to illustrate and not limit) one or more of acetaminophen, buspirone, an opioid(s) including pethidine (meperidine), dexmedetomidine, fentanyl, propofol, paralytic medication like vecuronium, a general anaesthetic(s)),
   (θ) in some embodiments the subject is a human and in alternative embodiments the subject is a non-human species, preferably a mammal;
in some embodiments a compound(s) of Formula (I) is administered (and/or a pharmaceutically-acceptable salt, solvate, hydrate or prodrug thereof, and/or a pharmaceutical composition thereof).
   Some Pharmaceutical Compositions of this Disclosure

A pharmaceutical composition comprising at least one compound (and/or a pharmaceutically-acceptable salt, solvate, hydrate or prodrug thereof) as defined in Formula [X](optionally at least one compound of at least one of Formula (I), (II), (III), (IV), (V), (VI), (VII), (VIII)) and at least one of a pharmaceutically-acceptable carrier(s), additive(s), diluent(s);
optionally wherein the pharmaceutical composition confers modified/controlled/extended/sustained/prolonged/slowed/delayed/pulsed/pulsatile/accelerated/fast/target ed/programmed release of the compound(s) when administered to a subject, optionally such that its constituent compound(s) amount/dose causes a smaller maximal drop, or no drop, in the subject's body temperature (but wherein the duration of the subject's body temperature drop might be extended, all be it at less amplitude: i.e. possibly less in amplitude and more in duration) than if this same/equivalent compound(s) amount/dose was administered to the subject alone or in an uncontrolled release pharmaceutical composition;
optionally wherein the pharmaceutical composition confers temperature controlled release of the compound(s) when administered to a subject,
optionally wherein the pharmaceutical composition only/preferentially releases compound(s) when subject body (or part(s) thereof) temperature is normal (e.g. within normal/typical/physiological limits range for the subject) and/or higher than normal, optionally wherein it is higher because of exogenous heating of the subject's entire body (e.g. in a device for conferring heat/hyperthermia therapy e.g. as sometimes used clinically for anti-cancer therapy) or body part(s), wherein a body part(s) where drug release is desired, e.g. in a cancer/tumor, is exogenously heated by a method(s) of the art (e.g. by incident electromagnetic radiation),
optionally wherein it is higher because of fever,
optionally wherein it is higher at a pathologically elevated temperature;
optionally wherein the pharmaceutical composition only/preferentially releases compound(s) in vivo at one or more of >48° C., >47° C., 46° C., >45° C., >44° C., >43° C., >42° C., >41° C., >40° C., >39° C., >38° C., >37° C., >36° C., >35° C., >34° C., >33° C., >32° C., >31° C., >30° C.;
optionally wherein the pharmaceutical composition only/preferentially releases compound(s) when subject body (or part(s) thereof) temperature is normal (e.g. within normal/typical/physiological limits range for the subject) and/or higher than normal, such that when the composition is administered to the subject it releases compound(s), which reduces subject body (or part(s) thereof) temperature, such that less/no compound(s) is then released per unit time and so the reduction in subject body (or part(s) thereof) temperature is slowed/abated and/or subject body (or part(s) thereof) temperature rises, if it rises enough then more compound(s) is released per unit time, and the loop iterates again one or more times such that subject body (or part(s) thereof) temperature drop is less in amplitude than if this same/equivalent compound(s) amount/dose was administered to the subject alone or in an uncontrolled release pharmaceutical composition;
optionally wherein the pharmaceutical composition comprises/includes one or more of biocompatible thermosensitive polymer (optionally with polysaccharide(s) to modulate the drug encapsulation and release efficiency), which undergoes a volume change at a temperature exceeding its phase/volume transition temperature, which releases the compound(s), preferably wherein the volume change is reversible if the temperature subsequently falls below the phase/volume transition temperature, preferably wherein the phase/volume transition temperature is tuned/set (optionally by tuning copolymerization conditions and by varying the content of repeating units in the copolymer) to be at normal subject body temperature or at a higher temperature, for example at a pathologically elevated body temperature, and/or at a tumor temperature and/or at an exogenously heated body/body part(s) temperature;
optionally wherein the pharmaceutical composition confers a trigger, wherein the trigger is one or more exogenously controlled stimuli (e.g. selected from temperature, ultrasonic, electronic etc.), controlled release of the compound(s) when administered to a subject;
optionally wherein the pharmaceutical composition confers a trigger, wherein the trigger is one or more cancer associated stimuli, controlled release of the compound(s) when administered to a subject;
optionally wherein the pharmaceutical composition confers pH controlled release of the compound(s) when administered to a subject, optionally only/preferentially releasing compound(s) when in an acidic environment, wherein extracellular acidity is a hallmark of cancers using Warburg metabolism; optionally wherein the pharmaceutical composition confers dual temperature and pH controlled release of the compound(s) when administered to a subject,
optionally only/preferentially releasing compound(s) when the composition is in a body area hotter than normal subject body temperature and in an acidic environment;
optionally wherein the pharmaceutical composition comprises/includes one or more of temperature responsive nanoparticle, thermosensitive hydrogels/nanogel, liposome, temperature sensitive liposome, heat-activated liposome (lysolipid thermally sensitive liposome) as used in Thermadox® (optionally used with exogenous heating by one or more of radiofrequency thermal ablation {RFA}, microwave hyperthermia, or high-intensity focused ultrasound {HIFU}), thermosensitive micelle, polymeric micelle, core shell structure, core shell microgel particle, thermoresponsive composite film, smart three dimensionally ordered porous material, thermosensitive microcontainer, nanoscale delivery vehicle; optionally wherein the pharmaceutical composition is distributed/sold/administered with a verbal/written communication (optionally in a paper insert/leaflet in a packet(s) containing the composition(s) {optionally called “instructions for use”, and/or “prescribing information” and/or “patient information leaflet”}) that its administration to a subject can cause their body temperature to drop [optionally informing that this is especially the case if a large/excess (e.g. more than the recommended) dose(s) is administered, optionally informing that this is especially the case if the subject is a child/baby (optionally informing that it is unsuitable for children and/or babies {optionally with the caveat that it can be administered to them if they are in a temperature controlled environment e.g. infant incubator/radiant warmer})] and optionally with one or more instructions to carry out should this body temperature drop happen to the subject (e.g. the subject should wear more clothes, wear warmer clothes, locate in a hotter environment, tell a doctor or pharmacist, go to a hospital) and/or optionally informing that the subject should/must minimize/stop alcohol intake (and/or other drug(s) intake that can affect thermoregulation such as a phenothiazine {like chlorpromazine etc.}, thioxanthenes etc.) for a period if this pharmaceutical composition is administered to the subject;
optionally wherein the pharmaceutical composition also comprises/includes one or more of an uncoupler (an uncoupler is a molecule that can bind a proton(s) in the mitochondrial intermembrane space (IMS), move across the mitochondrial inner membrane, and release the proton(s) in the mitochondrial matrix, which dissipates the proton motive force (pmf), and that can then return to the IMS, and repeat this sequence iteratively), optionally wherein the body temperature reduction drive in a subject that administration of the pharmaceutical composition causes, because of its componentry F₁F₀ ATP hydrolysis inhibitor(s)/reducer(s), is completely/partially cancelled/offset by the body temperature increase drive caused by a componentry uncoupler(s) in the same pharmaceutical composition, such that the subject's body temperature change is smaller in absolute value (optionally zero) than if either the amount of F₁F₀ ATP hydrolysis inhibitor(s) or uncoupler(s) was administered alone and/or in the pharmaceutical composition without the other component, optionally wherein a componentry F₁F₀ ATP hydrolysis inhibitor compound is also an uncoupler, optionally wherein these two opposite subject body temperature modifying aspects of the same compound completely or partially cancel such that the subject's body temperature is not much changed, if at all, by administration of this pharmaceutical composition, optionally wherein a combination of F₁F₀ ATP hydrolysis inhibitor(s) and uncoupler(s) compounds in the same pharmaceutical composition exerts greater anti-cancer activity when administered to a subject than if the same amount of either was administered without the other in the pharmaceutical composition, optionally wherein the combination amount of an F₁F₀ ATP hydrolysis inhibitor(s) and uncoupler(s) compounds in the same pharmaceutical composition is synergistic for anti-cancer activity; optionally wherein the pharmaceutical composition also comprises/includes one or more of a compound(s) that inhibits UCP2 (e.g. genipin and/or cisplatin);
optionally wherein the pharmaceutical composition also comprises/includes one or more of a cyclodextrin(s);
optionally wherein the pharmaceutical composition also comprises/includes one or more of a fatty acid(s);
optionally wherein the pharmaceutical composition is distributed/sold/administered with a verbal/written communication (optionally in a paper insert/leaflet in a packet(s) containing the composition(s) (optionally called “instructions for use”, and/or “prescribing information” and/or “patient information leaflet”)) that it should not be administered to a female subject who is pregnant (optionally delimiting this to early pregnancy, optionally to the first two months or first month or first 3 weeks or first 2 weeks or first week or for a number of days that is less than the number of days in 2 months) and/or who is trying/wants to get pregnant over the period of administration and/or that it should not be administered within the early days and/or weeks of a subject's pregnancy and/or in the early days (e.g. up to one of 1, 2, 3, 4, 5, 6, 7, 8, 9, 10, 11, 12, 13, 14, 15 days later) and/or weeks after having (optionally unprotected) sex with the intention/want (at the time and/or retrospectively) of getting pregnant;
in some embodiments a compound(s) of Formula (I) (and/or a pharmaceutically-acceptable salt, solvate, hydrate or prodrug thereof) is part or all of the pharmaceutical composition.
   Using a Compound of this Disclosure to Appraise the Efficacy of a Drug Delivery Technology

A method comprising:
measuring the amplitude and/or duration of body temperature drop (if any) caused in a subject by their being administered one or more compounds according to Formula [X] {optionally at least one compound of at least one of Formula (I), (II), (III), (IV), (V), (VI), (VII), (VIII)} and/or a pharmaceutically-acceptable salt, solvate, hydrate or prodrug thereof, and/or a pharmaceutical composition thereof, and/or a selective/preferential F₁F₀ ATP hydrolysis inhibitor compound(s)/composition(s) (that preferably inhibits F₁F₀ ATP synthesis less or, more preferably, not at all), wherein the compound(s)/composition(s) can be administered by a drug administration route/device/technology (e.g. transdermal skin patch), optionally in a pharmaceutical composition, wherein the amplitude and/or duration of body temperature drop (and optionally what amplitude and/or duration occurs in different parts of the body e.g. viewed by thermal imaging) reports on the efficacy/associated pharmacokinetics of one or more of the drug administration route/device/technology/composition selected, which can inform upon the merit(s) (or lack thereof) of this/these for effective/desirable administration of this compound(s) to the subject, optionally wherein different routes and/or devices/technologies of drug administration and/or different pharmaceutical compositions are trialed, wherein this can particularly report on the merit(s) (or lack thereof) of a pharmaceutical composition(s) for effective/desired characteristic(s) of drug administration, optionally wherein this is leveraged in experiment(s) to research/test/tune/optimize/select/design/improve the component(s) of a pharmaceutical composition to deliver effective/desired characteristic(s) of drug administration in a subject, optionally wherein the amplitude and/or duration of body temperature drop can be measured by the energy (e.g. by ambient temperature and/or electromagnetic wave {e.g. infra-red, e.g. IR-A e.g. from 0.78 to 1.4 μm wavelength}heating, preferably wherein the amount of electrical energy the heating device(s) uses is monitored) that needs to be inputted into the subject body to keep their body temperature constant, or nearly so, and/or at a life permissive body temperature, optionally wherein adaptive heating is utilized, which adjusts the heating element output {e.g. an infrared lamp, radiant warmer, incubator or any other heating element(s)} in response to the measured body temperature {e.g. measured by rectal temperature probe or by thermal imaging, or any other body temperature measuring/recording device(s)};
it is novel for the effective amount of a drug(s) in a subject body to have such an easy simple (optionally continuous e.g. if rectal probe is kept in continuously e.g. if thermal imaging is continuous) physiological readout, as in this case with body temperature {more typically to find the amount of a drug in a subject body, blood/plasma/serum samples are taken and analysed, wherein the result(s) doesn't necessarily report on the pharmacologically effective fraction, and the view is definitely not continuous but is of snapshots which require labour to take: more snapshots requires more work, more consumables consumed and greater disturbance of the subject};
any method that uses a drug(s) that causes a body temperature drop in a subject to research/test/tune/optimize/select/design/improve a pharmaceutical composition/formulation and/or a drug(s) delivery route/device/technology is componentry to this method;
this method also encompasses its use with one or more of a candidate/trial/novel (as yet unproven) pharmaceutical composition(s), drug(s) administration/delivery route(s)/device(s)/technology(s);
an optional step of this method is to heat the pharmaceutical composition to be at or close to the normal body temperature of the subject before it is administered to the subject;
in some embodiments a compound(s) of Formula (I) is used (and/or a pharmaceutically-acceptable salt, solvate, hydrate or prodrug thereof, and/or a pharmaceutical composition thereof) in this method.

### DETAILED DESCRIPTION OF THE DRAWINGS

For purposes of clarity, not every component is labelled in every figure, nor is every component of each embodiment of this disclosure shown where illustration is not necessary to allow those of ordinary skill in the art to understand the disclosure.

Herein incorporated by reference, in entirety, are the Drawings, and corresponding figure legends, of PCT/EP2018/069175 (published as WO2019/012149A1) and Canadian application number 3,050,553, and the experimental data, and associated writing/analysis/explanation, in applicant's reply to the “written opinion of the International Search Authority” for PCT/EP2018/069175, which is publically available on the European Patent Register file for the EP entry of this PCT: EP application number 18746115.7 [published as EP3652156].

Inhibiting/Reducing F₁F₀ ATP Hydrolysis in Cancer Cells Exerts Anti-Cancer Activity

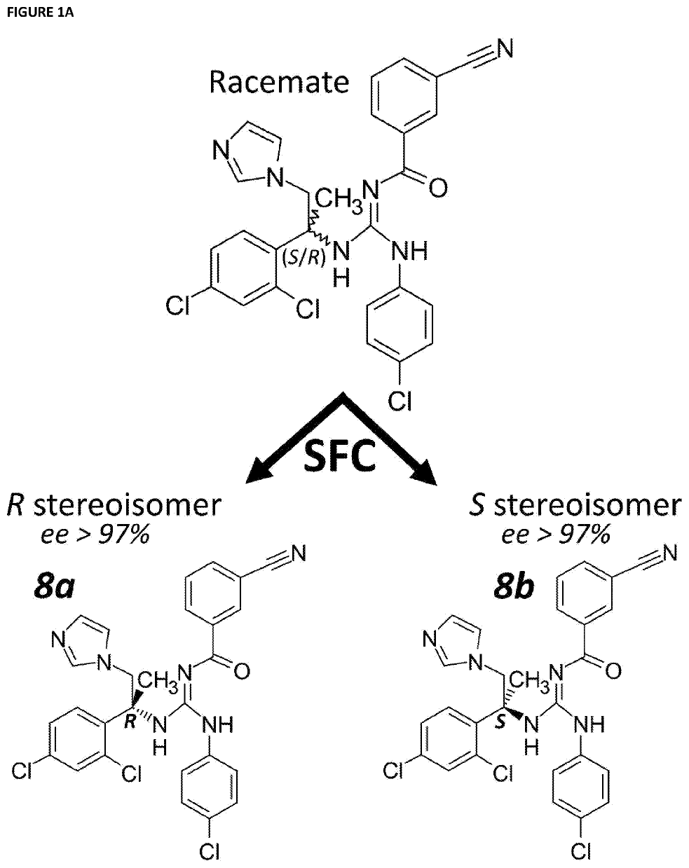

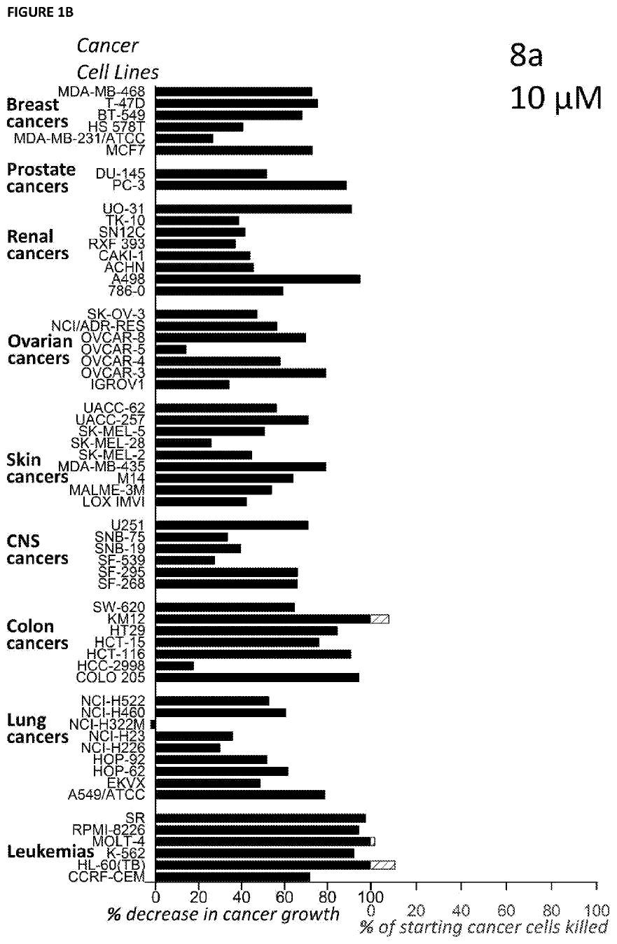

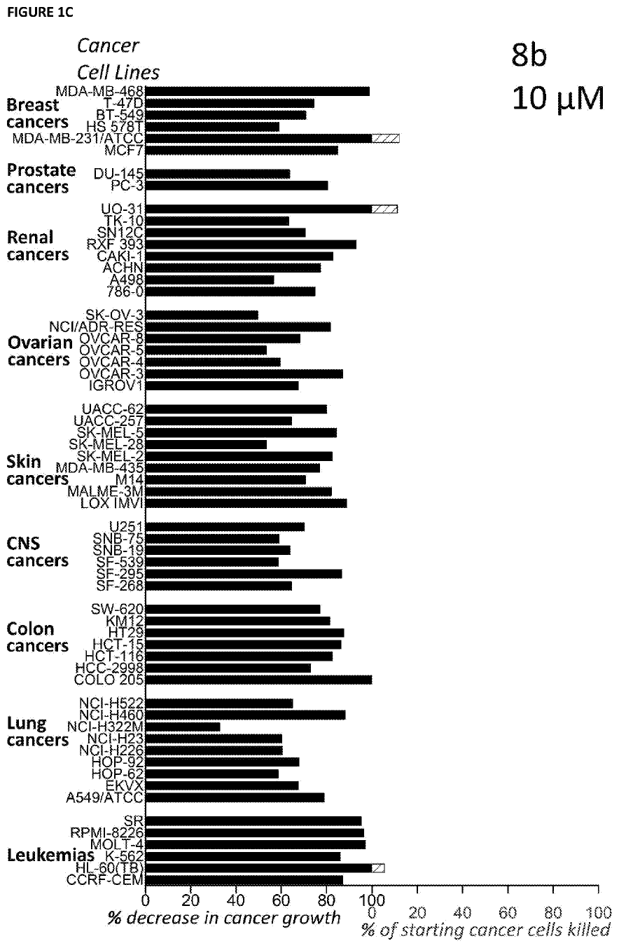

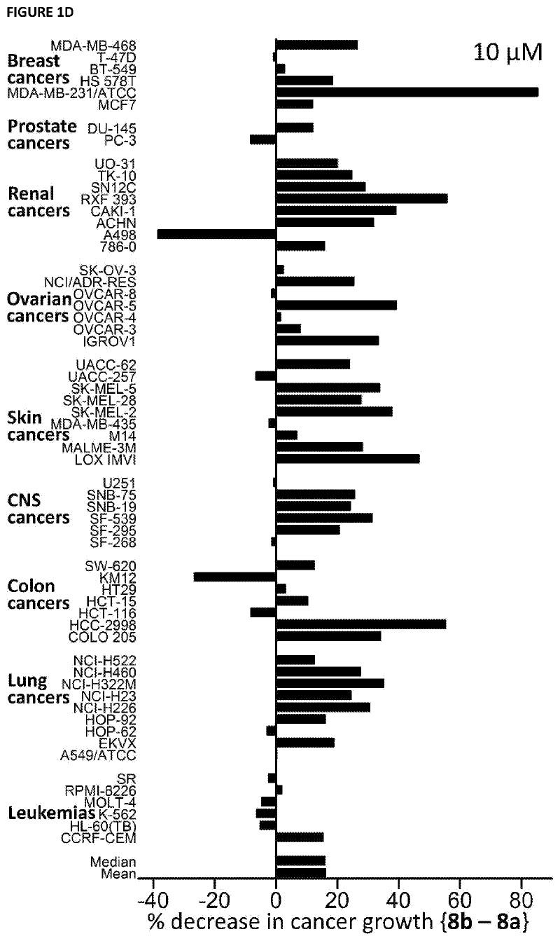

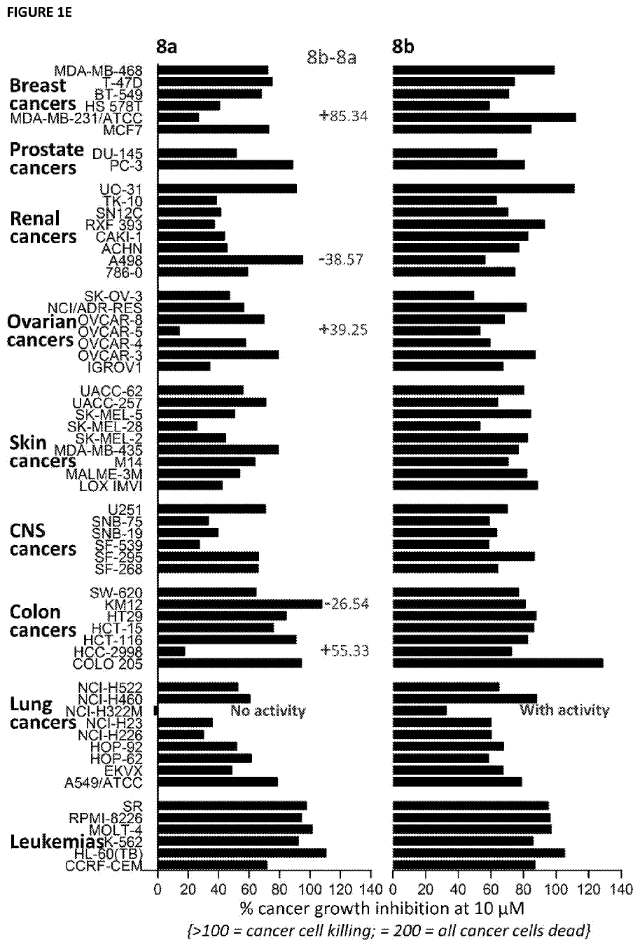

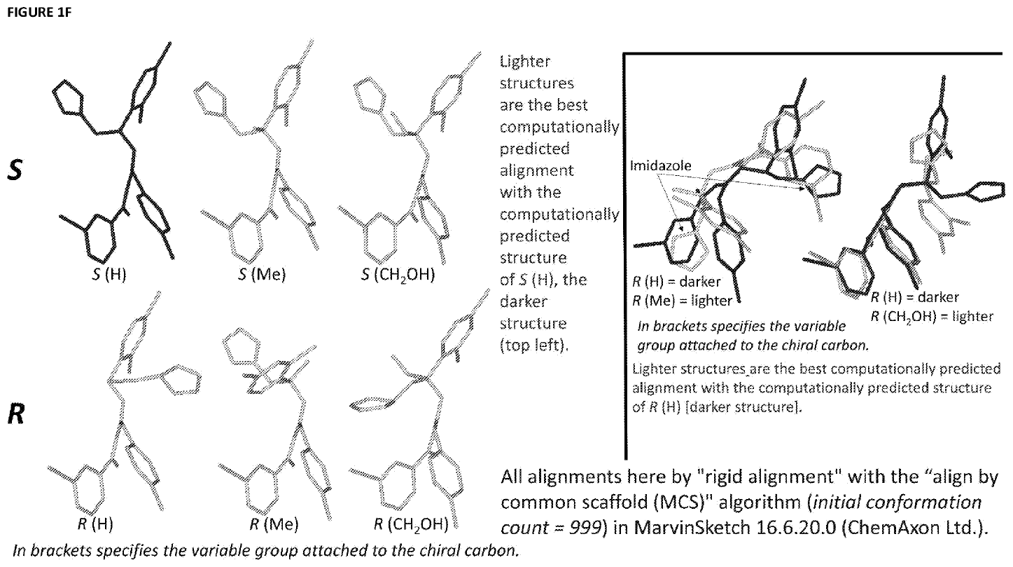

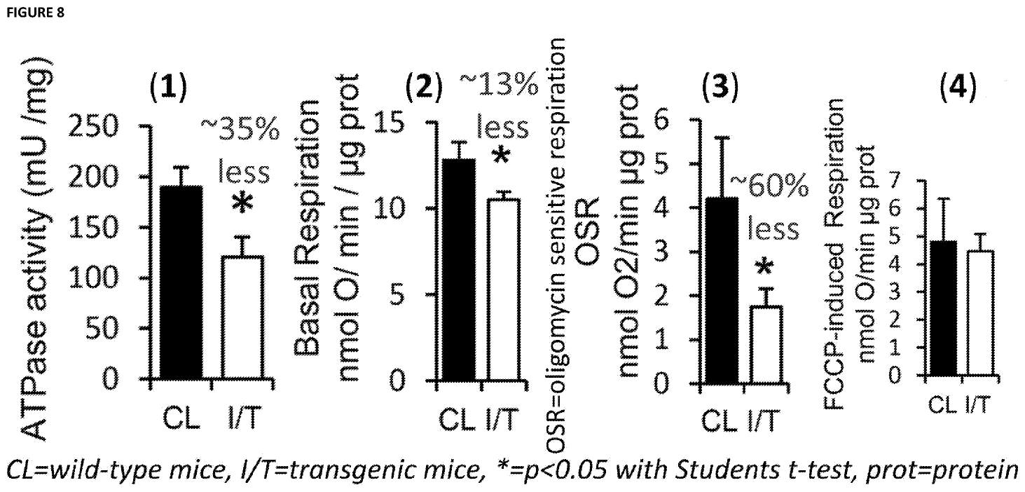

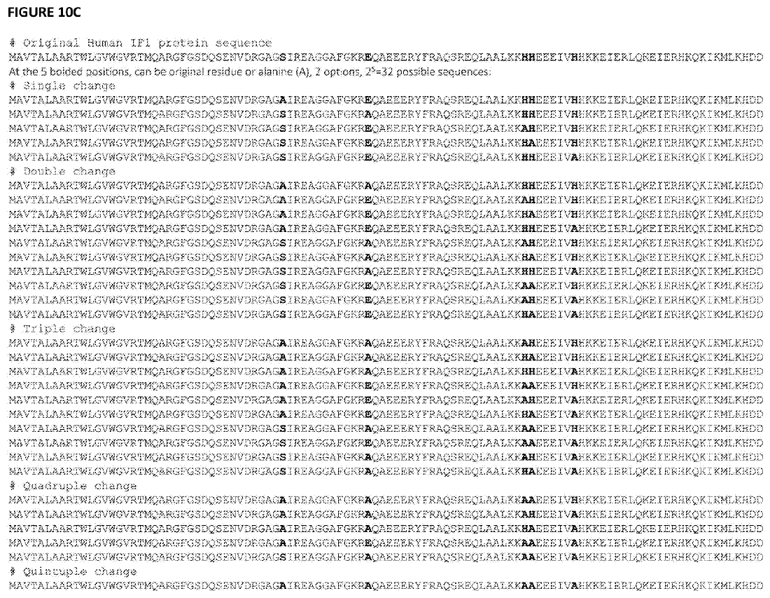

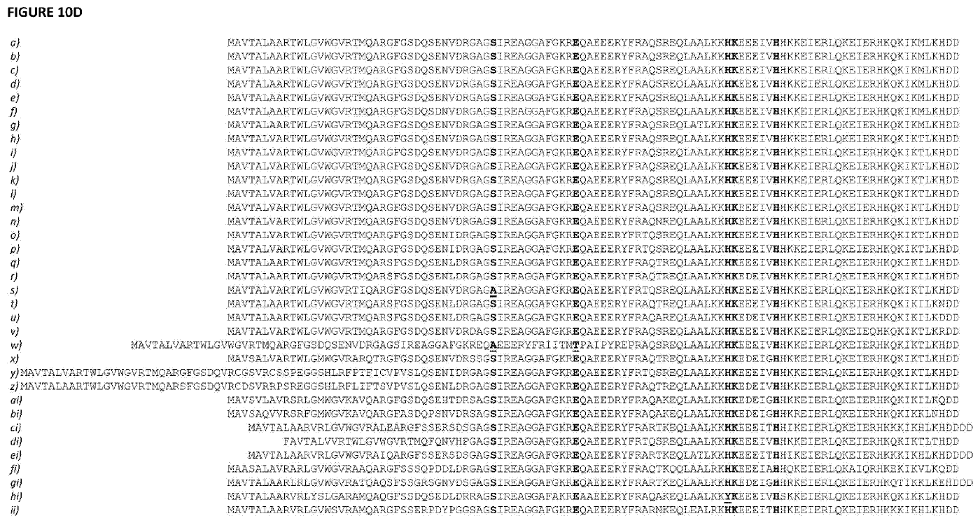

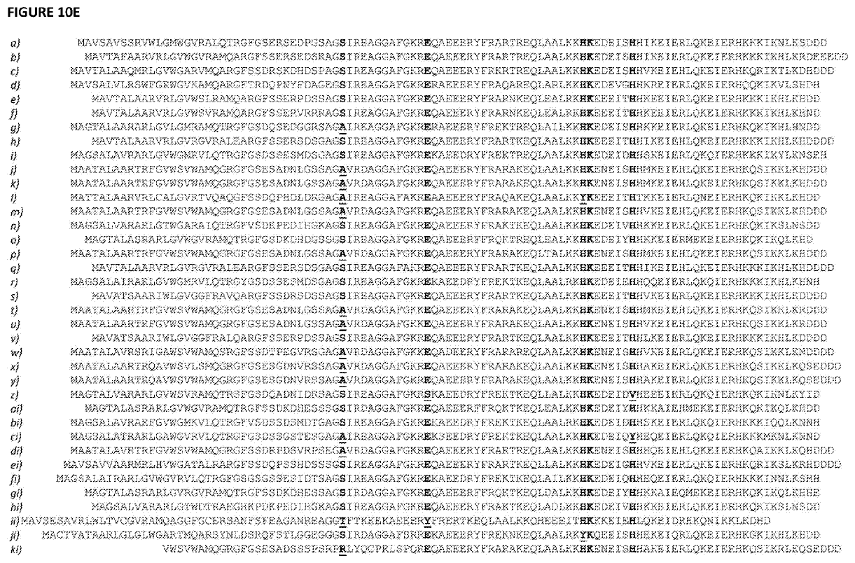

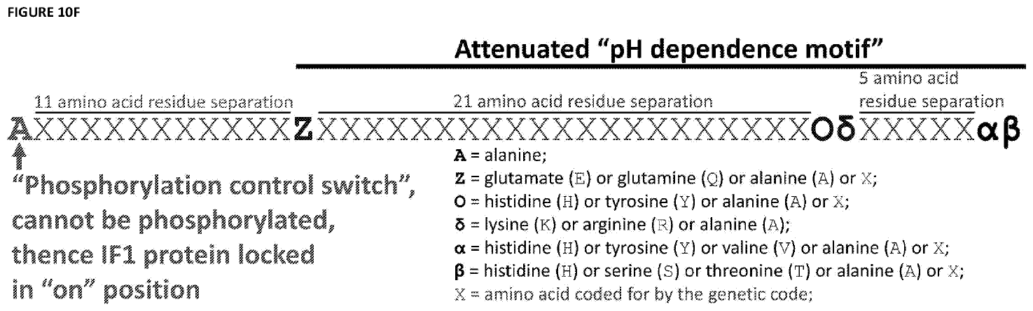

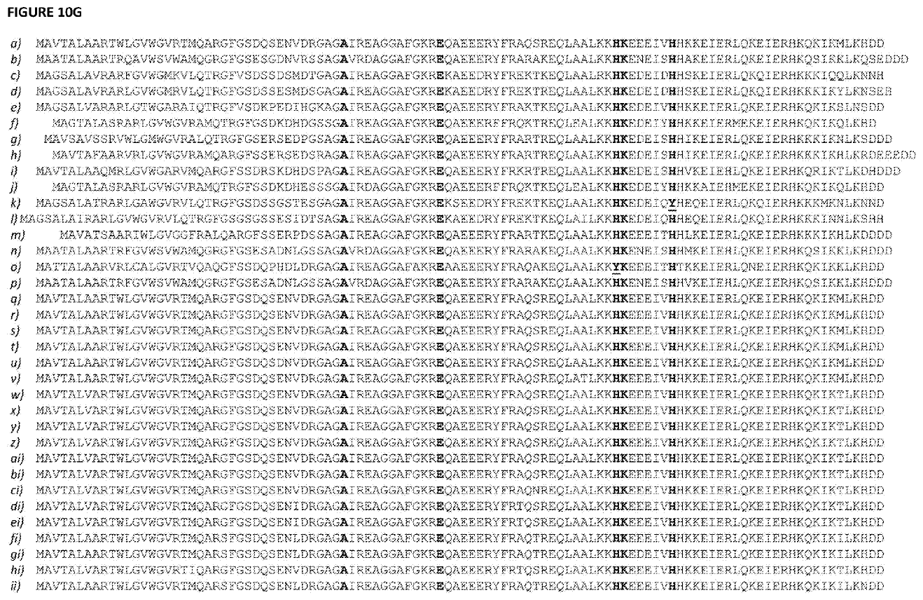

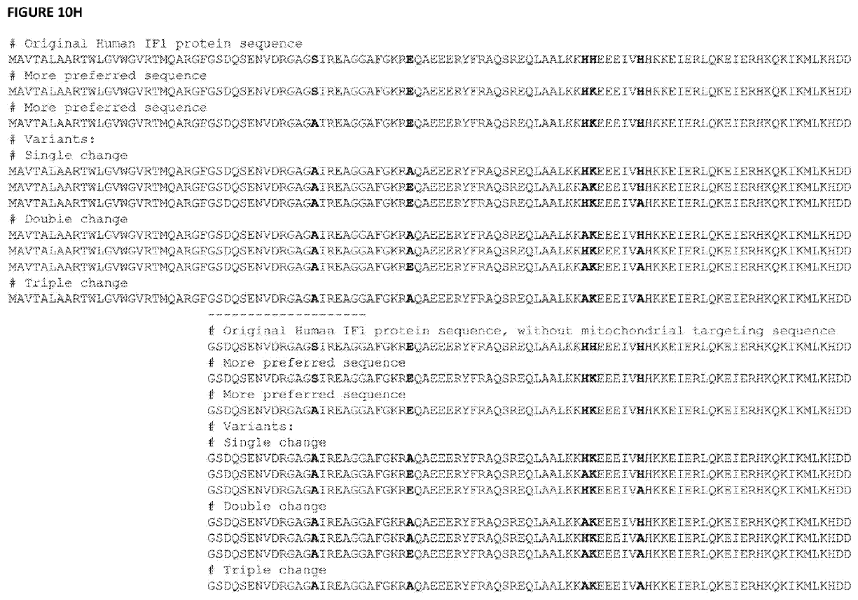

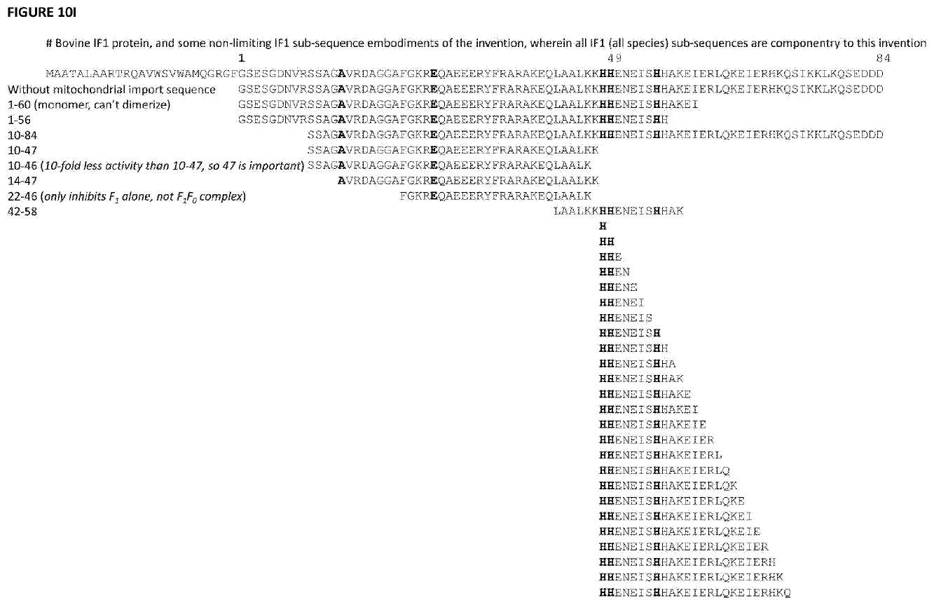

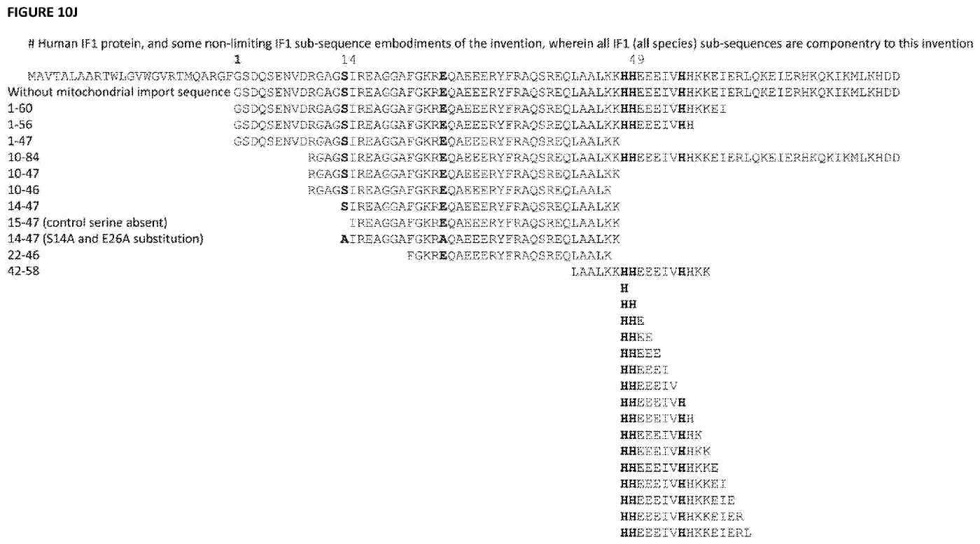

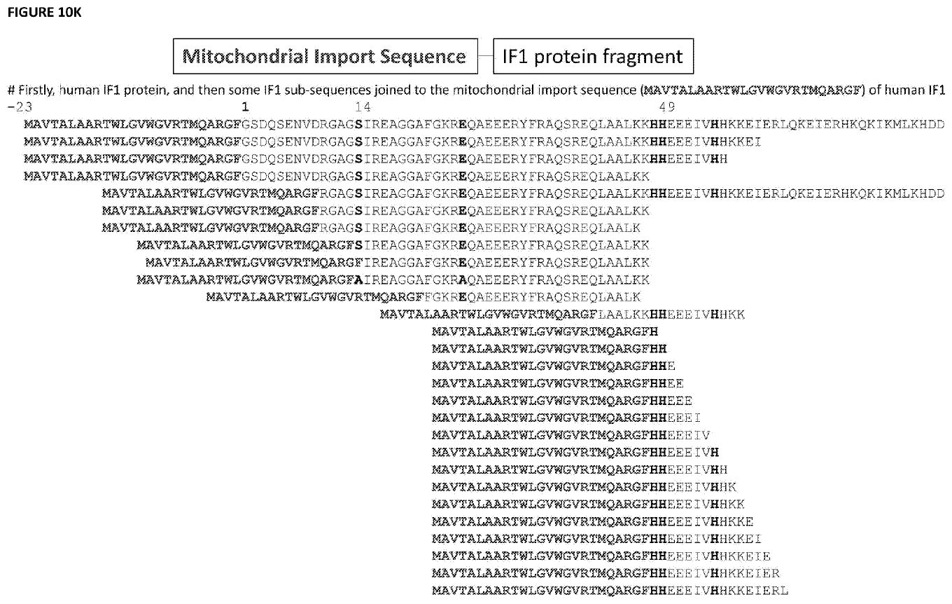

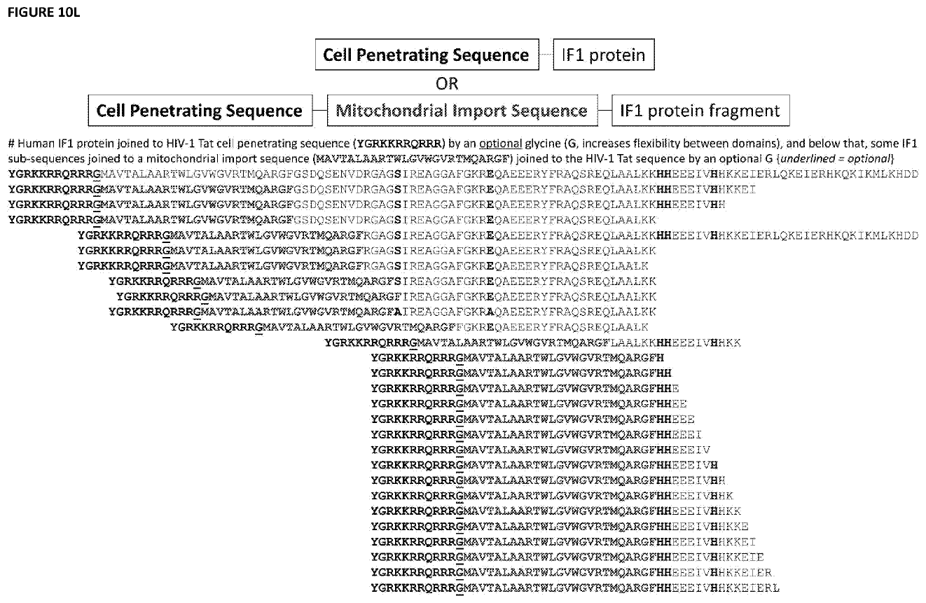

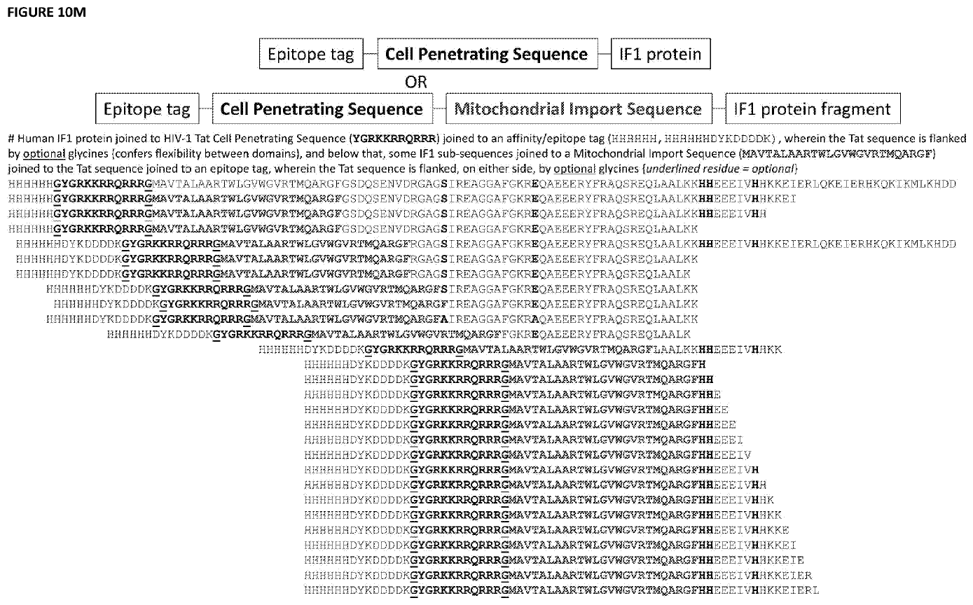

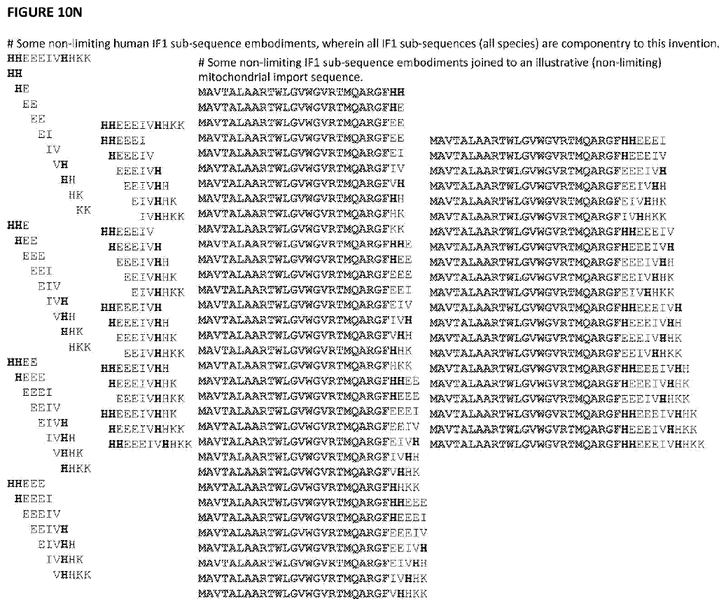

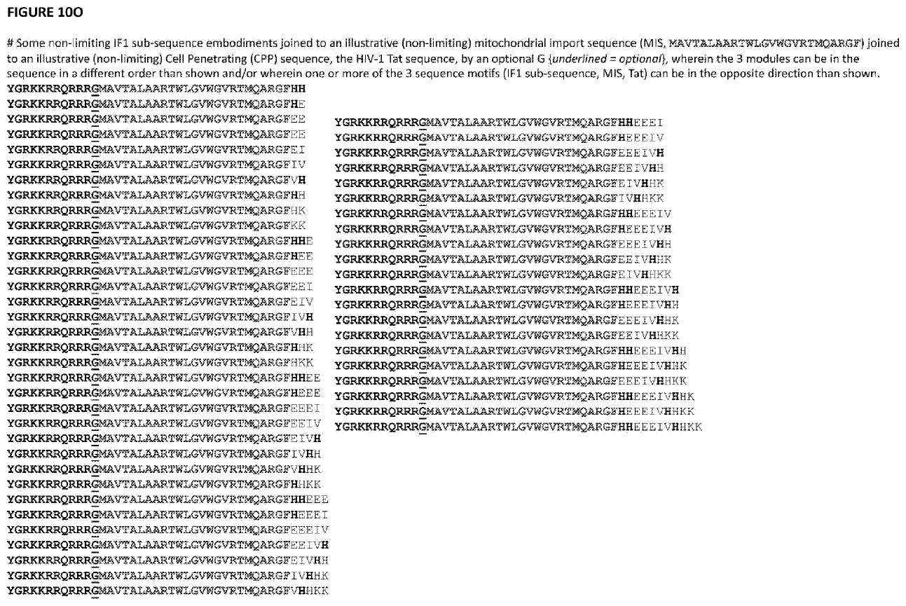

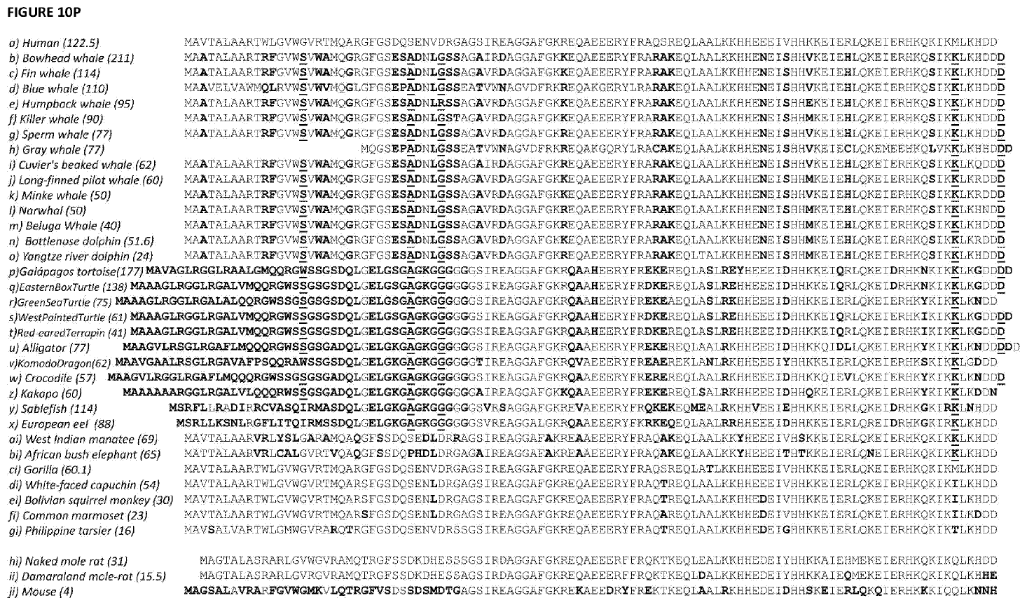

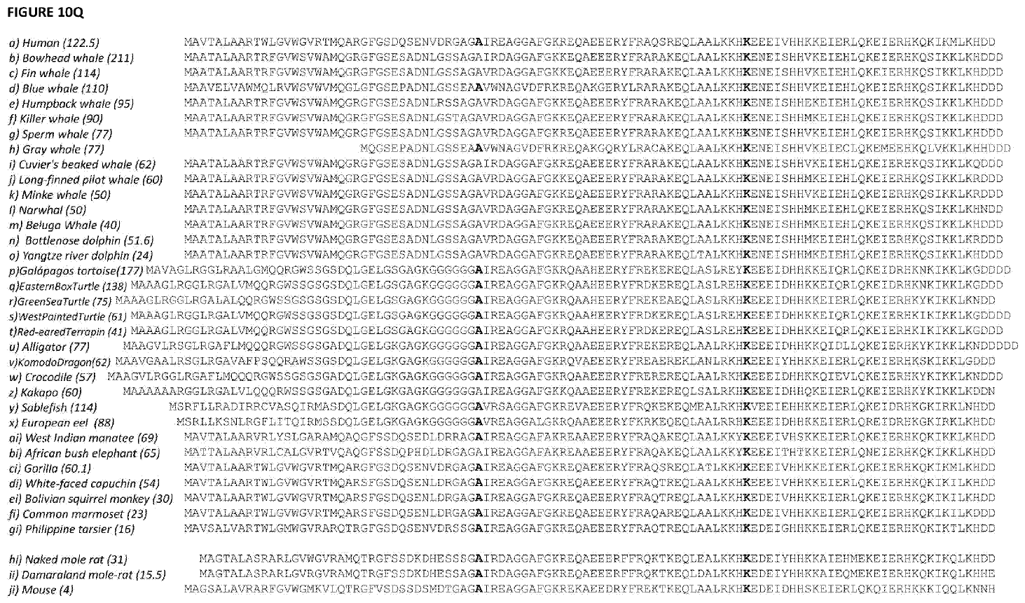

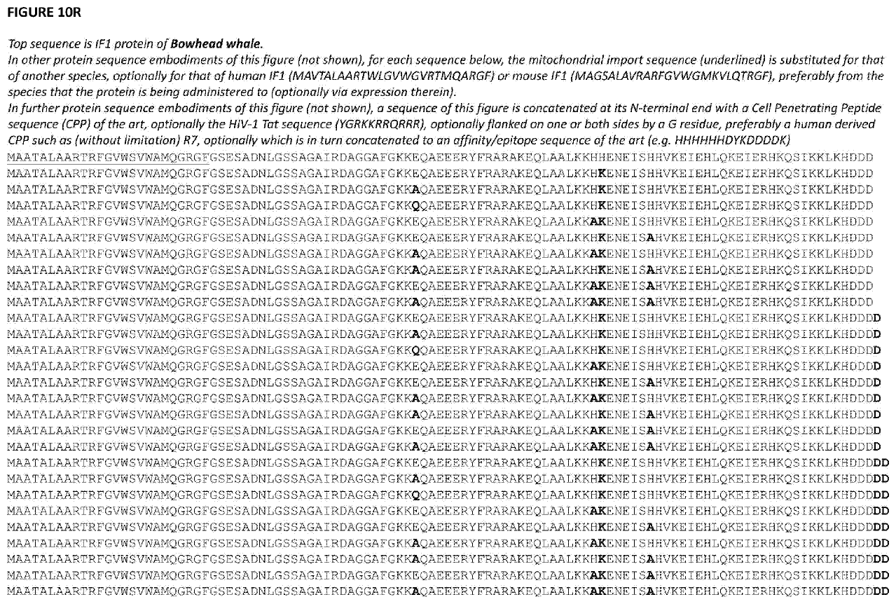

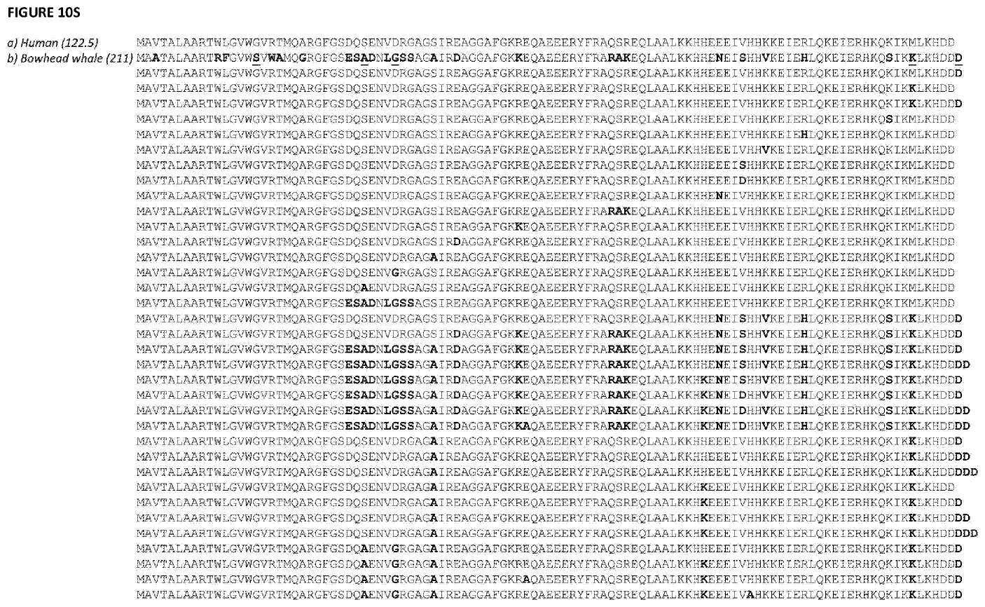

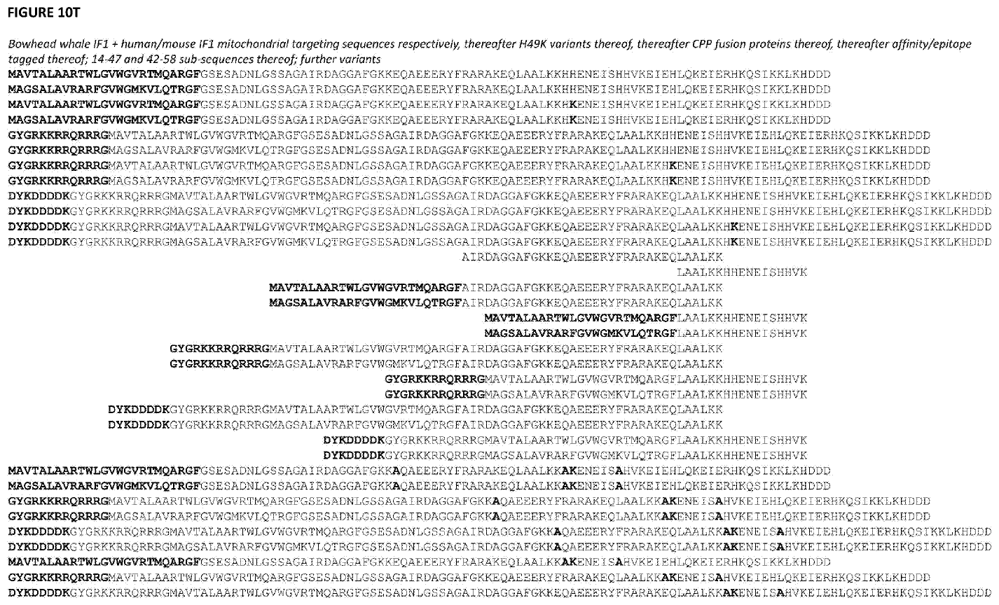

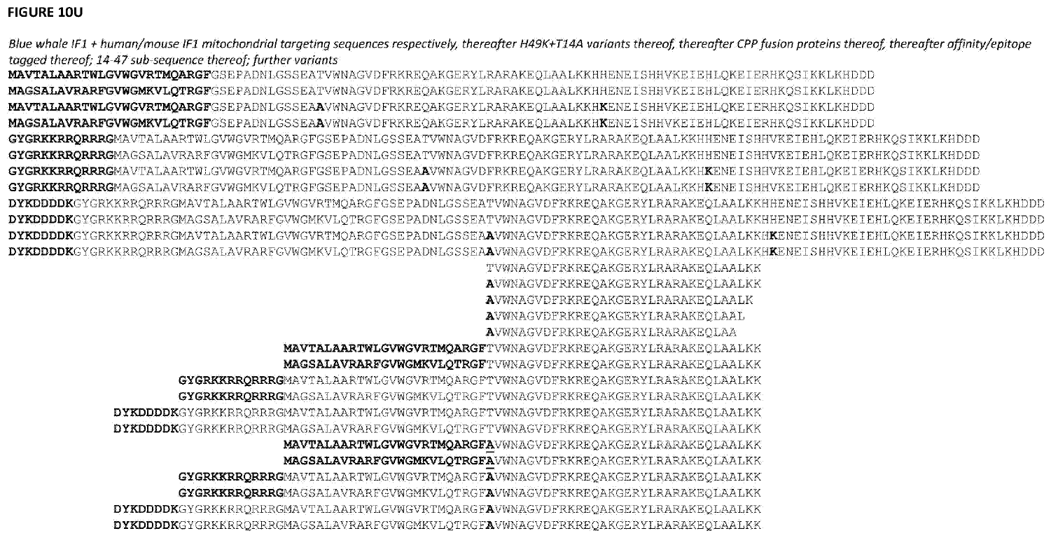

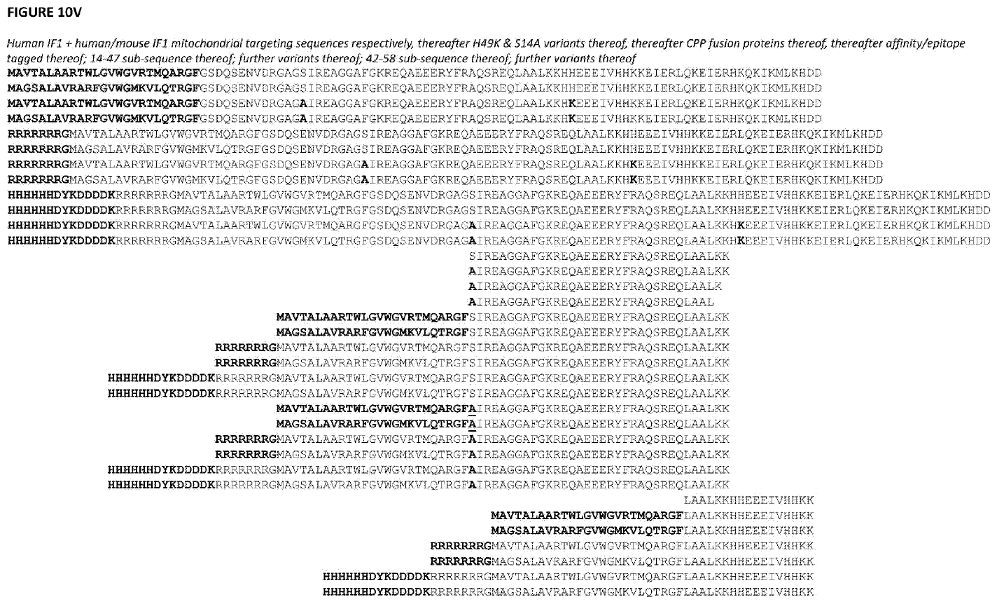

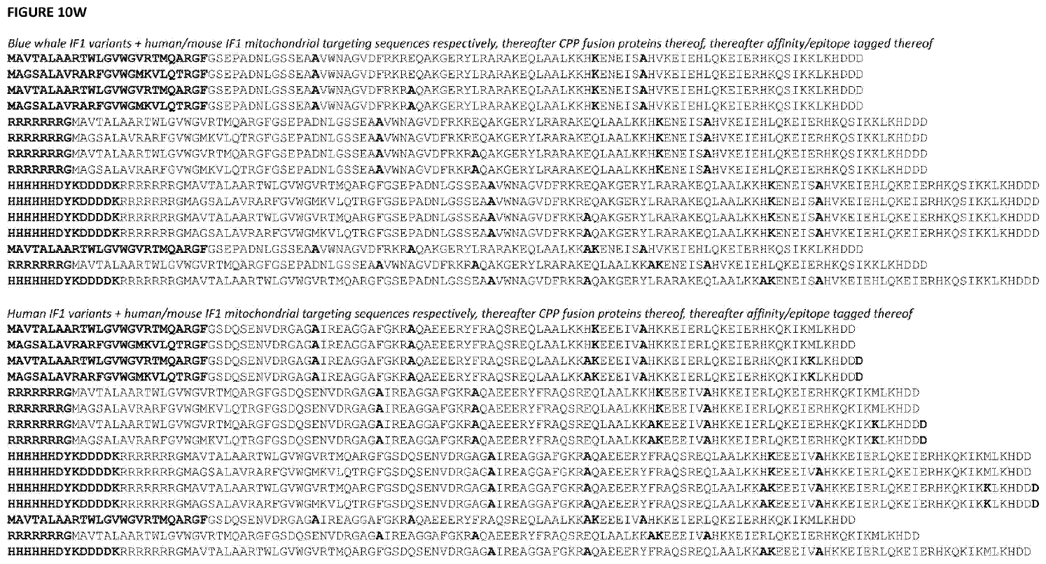

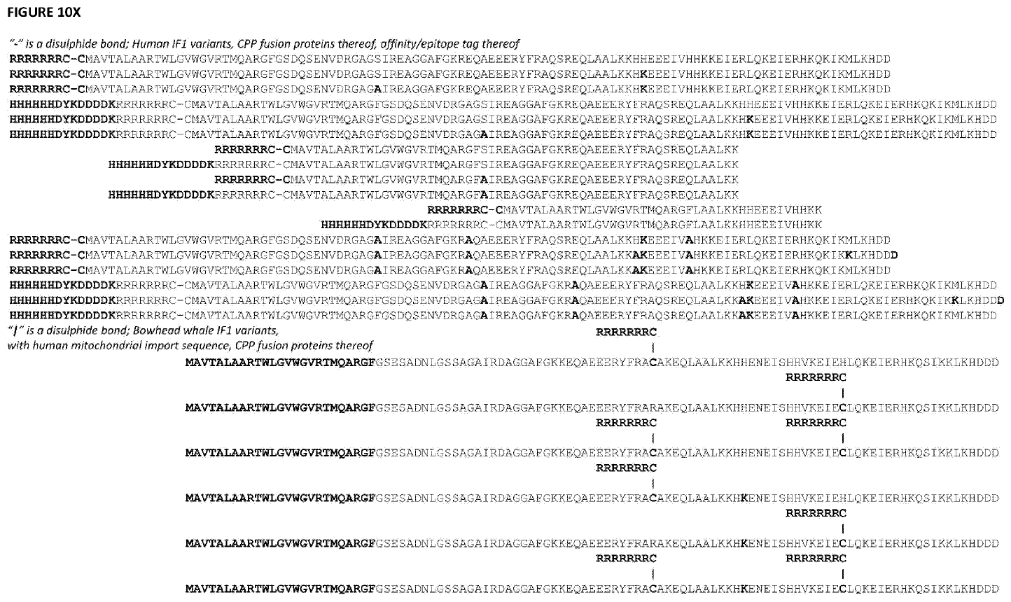

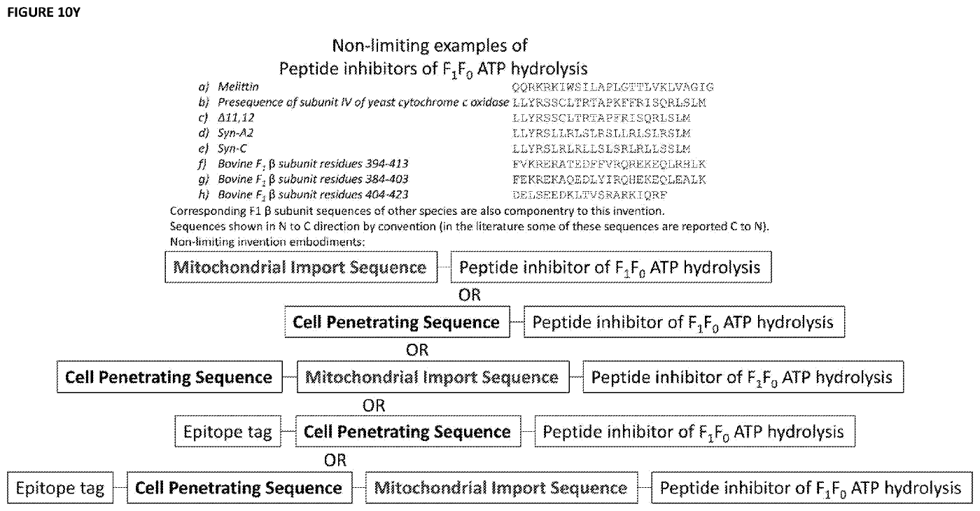

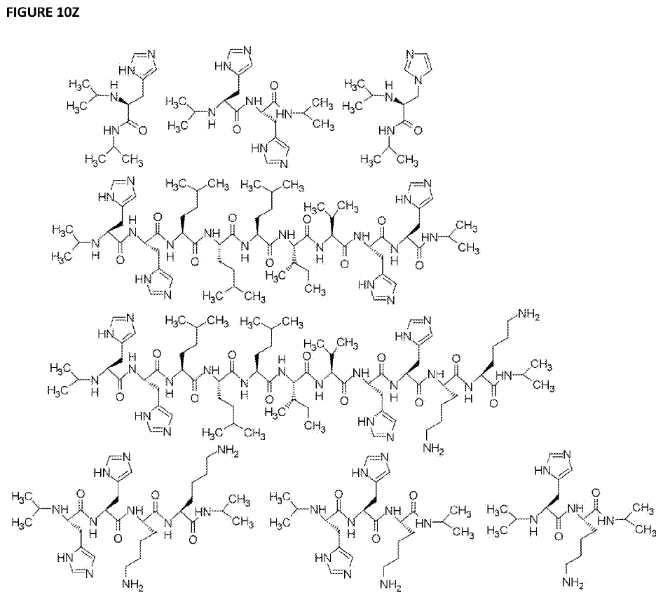

FIG. 8 in PCT/EP2018/069175 (and also FIG. 8 in Canadian application number 3,050,553) shows the structure of compounds 6a and 6b, which are opposite stereoisomers (R and S respectively) with a hydrogen on their chiral carbon, wherein 6b can potently inhibit/reduce F₁F₀ ATP hydrolysis and 6a cannot, wherein they are both shown to have anti-cancer activity in vitro in NCI-60 anti-cancer testing, wherein 6b exerts anti-cancer activity by reducing reduce F₁F₀ ATP hydrolysis in cancer cells, and 6a exerts anti-cancer activity by epimerizing to be 6b in a biological system. FIG. 10 in Canadian application number 3,050,553 (and also applicant's reply to the “written opinion of the International Search Authority” for PCT/EP2018/069175) shows the structure of compounds 7a and 7b, which are the same as 6a and 6b respectively, except that they have deuterium (enrichment) instead of hydrogen on their chiral carbon, and so by the Kinetic Isotope Effect (KIE) their racemization rate is slower, wherein 7b is shown to have greater anti-cancer activity than 6b, because 7b epimerizes to 7a more slowly than 6b epimerizes to 6a, and 7a is shown to have less anti-cancer activity than 6a, because 7a epimerizes to 7b more slowly than 6a epimerizes to 6b. FIG. 11 in Canadian application number 3,050,553 shows the structure of compounds 8a and 8b, which are the same as 6a and 6b respectively, except that they have methyl instead of hydrogen on their chiral carbon, wherein because these compounds cannot interconvert by racemization, it would be expected for 8b to have potent, and for 8a to have little, anti-cancer activity, but wherein surprisingly this isn't observed, wherein this figure is reproduced herein as FIG. 1.

FIG. 1: Chiral supercritical fluid chromatography (SFC) was used to separate the shown racemate into its component R and S stereoisomers and two samples of opposite >97% enantiomeric excess (ee) was achieved: termed 8a and 8b respectively. 8a and 8b differ from 6a and 6b because they have methyl (Me, CH₃), instead of hydrogen (H), upon their chiral carbon. 8a and 8b were independently tested in NCI one-dose (10 μM) testing [107-108]: their results are shown in FIGS. (1B) and (1C) respectively. The anti-cancer activity of 8a and 8b against the different cancer cell lines of the NCI-60 assay was correlated i.e. the greater, and lesser, of their anti-cancer activity was against the same cell lines (Pearson correlation: R =0.5669, significant at p<0.00001). This correlation is notably less than for 6a vs. 6b (0.7991) and 7a vs. 7b (0.8049).

(1D) Recasts data from FIGS. 1B and 1C. Null hypothesis: equal (0.5) probability that x-axis value is positive (+ve) or negative (−ve): that 8a or 8b is the more powerful stereoisomer for any given cell line. Binomial probability of observed number of +ve (45) and −ve (15) {n=60}=0.00004613852. P-value (one-tailed) for 45 or more +ve (n=60)=0.000091. Conclusion: 8b has greater anti-cancer activity than 8a at 10 μM. But one can see that there are notable exceptions, which are discussed now with 1E.

(1E) 8a and 8b NCI one-dose (10 μM) test results side by side. % cancer growth inhibition >100 means there are less cancer cells at experiment end than beginning (i.e. cancer killing activity), =200=all cancer cells dead at experiment end. The observed pattern of 8a vs. 8b activity is hard to explain. One would expect one stereoisomer to have greater or equal activity than the other. Whereas here, 8b mostly has greater activity, and in some cases much greater (e.g. Δ=85.34% with MDA-MB-231/ATCC), but in some cases 8a has the much greater activity (e.g. Δ=38.57 with A498). Moreover, for one cancer cell line (NCI-H322M), 8a has no activity, yet 8b does. While not wishing to be bound by theory, the following model can explain these results, wherein the anti-cancer activity of 8a and 8b is set by (1) the cancer's sensitivity to a specific F₁F₀ ATP hydrolysis inhibitor, which can vary between different cancer cell lines, and (2) the activity of an enzyme(s) that hydroxilates the methyl group (CH₃) of 8a and 8b to CH₂OH, which can vary between different cancer cell lines, wherein this enzyme(s) is referred to herein as CYP, but it needn't necessarily be a Cytochrome P450 enzyme(s) as other hydroxylase/monooxygenase enzymes are known to those of the art:

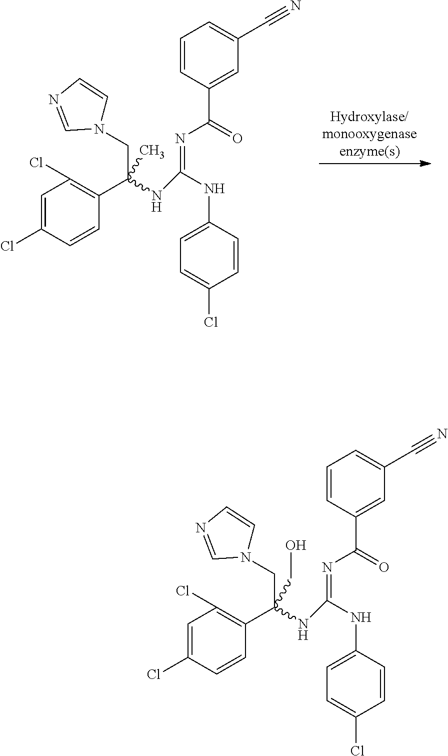

Moreover, the ability to exert specific F₁F₀ ATP hydrolysis inhibition, and thence anti-cancer activity, is ranked: R (CH₂OH)>S (CH₂OH)≈S (Me)>R (Me), wherein R/S refers to stereochemistry and the group in brackets is the group on the chiral carbon. If CYP activity is low or non-existant, R (Me) and S (Me) are the predominant intracellular species of 8a and 8b respectively, and so, for anti-cancer activity, 8a<8b. If CYP activity is high, R (CH₂OH) and S (CH₂OH) are the predominant intracellular species of 8a and 8b respectively, and so, for anti-cancer activity, 8a >8b. It is hard to delinearate which of S (CH₂OH) and S (Me) has the greater F₁F₀ ATP hydrolysis inhibitory, and thence anti-cancer, activity. S (Me) does have anti-cancer activity, as observed with MDA-MB-231/ATCC, when 8a activity is much lower than 8b and so CYP activity must be low, thence S (Me) predominates. Yet S (CH₂OH) does have anti-cancer activity because 8b can still exert anti-cancer activity when 8a can. The NCI-H322M cancer cell line, against which 8a has no anti-cancer activity, may have a mutation in, and/or especially low expression of, the relevant CYP enzyme(s), wherein this prevents it from hydroxylating the methyl of R (Me) and S (Me).

(1F) can explain this model more. 6a, R (H), and 6b, S (H), cannot and can potently inhibit F₁F₀ ATP hydrolysis respectively [8, 109]. This figure shows that S (Me) and S (CH₂OH) are likely to be very structurally similar to S (H), and thence likely to also potently inhibit F₁F₀ ATP hydrolysis, and to exert anti-cancer activity. Crucially, R (Me) and R (CH₂OH) are not likely to be very structurally similar to R (H), nor each other, and in the case of R (CH₂OH) this enables it to potently inhibit F₁F₀ ATP hydrolysis and exert anti-cancer activity. So, in this (CH₂OH) case, and in the priming CH₃ on chiral carbon case, R over S stereochemistry can, in some embodiments, be favoured/desirable.

So, 8b has greater anti-cancer activity than 8a against some cancer cell lines. But 8a has greater anti-cancer activity than 8b against some other cancer cell lines. In some embodiments, an amount of 8b (preferably a therapeutically effective amount), and/or a salt, solvate, hydrate or prodrug thereof, is administered to treat a subject with [or suspected to have, or at risk of] cancer (or to treat a subject with a different disease/disorder/condition [including aging] mentioned herein, i.e. which can be treated with an F₁F₀ ATP hydrolysis inhibitor compound). In other embodiments, an amount of 8a (preferably a therapeutically effective amount), and/or a salt, solvate, hydrate or prodrug thereof, is administered to treat a subject with [or suspected to have, or at risk of] cancer (or to treat a subject with a different disease/disorder/condition [including aging] mentioned herein, i.e. which can be treated with an F₁F₀ ATP hydrolysis inhibitor compound). In alternative embodiments, an amount of a racemate or scalemate of 8a and 8b (preferably a therapeutically effective amount), and/or a salt, solvate, hydrate or prodrug thereof, is administered to treat a subject with [or suspected to have, or at risk of] cancer (or to treat a subject with a different disease/disorder/condition [including aging] mentioned herein, i.e. which can be treated with an F₁F₀ ATP hydrolysis inhibitor compound).

Methyl (instead of hydrogen or deuterium) upon the chiral carbon was expected to block racemization and increase the anti-cancer activity of the S stereoisomer, and decrease the anti-cancer activity of the R stereoisomer. But this added methyl confers a site for metabolism, wherein the R stereoisomer of this metabolized structure can adopt a very different structure than that adopted by 6a and 7a, wherein this structure can actually inhibit F₁F₀ ATP hydrolysis. So, with 6a and 6b, and 7a and 7b, a clean demarcation of anti-cancer activity, and lack thereof, with the S and R stereoisomers respectively, isn't observed because of racemization in a biological system. With methyl on the chiral carbon, 8a and 8b cannot racemize but this methyl confers a target for metabolization which changes the adopted structure of the R stereoisomer, such that it can actually inhibit F₁F₀ ATP hydrolysis. In some embodiments, the S and/or R stereoisomer with CH₂OH on its chiral carbon is administered, wherein administration of just/disproportionally the R stereoisomer thereof is preferred (R stereoisomer in enantiomeric excess). R stereoisomer with CH₂OH on its chiral carbon can be produced, for non-limiting example, by administering 8a to in vitro (preferably human, e.g. liver) microsomes (which are commercially available [e.g. from Sigma-Aldrich, St. Louis, MO, USA {which is a subsidiary of Merck KGaA}, or similar]; preferably afforded NADPH e.g. by addition of a NADPH-generating system; microsome assays of metabolic modification of a compound are well known in the art e.g. refer [110-111], the entirety of which are incorporated herein by reference, many similar papers in the literature findable by one of the art) and retrieving the metabolized structure with CH₂OH on its chiral carbon (optionally retrieving using Solid Phase Extraction {SPE} and preparative High Perfomance Liquid Chromatography {HPLC}, optionally confirming identity with mass spectrometry, e.g. searching for m/z 567.086 [M+H]⁺, optionally further confirming with ¹H NMR e.g. searching for the absence of the distinctive methyl signature, which is 3 hydrogens with the same (or very similar) chemical shift [ppm], optionally with further separation by one or more of distillation [leveraging differential vapor pressure], extraction [leveraging differential solubility], crystallization [leveraging differential level of saturation in a solvent], reaction with a resolving agent). A sample with enantiomeric excess (preferably >70%, and more preferably >97%, ee) of the R stereoisomer, with CH₂OH on its chiral carbon, will be termed 9a. A sample with enantiomeric excess (preferably >70%, and more preferably >97%, ee) of the S stereoisomer, with CH₂OH on its chiral carbon, will be termed 9b. In some embodiments, an amount of 9b (preferably a therapeutically effective amount), and/or a salt, solvate, hydrate or prodrug thereof, is administered to treat a subject with [or suspected to have, or at risk of] cancer (or to a treat subject with a different disease/disorder/condition [including aging] mentioned herein, i.e. which can be treated with an F₁F₀ ATP hydrolysis inhibitor compound); in other, more preferred, embodiments, an amount of 9a (preferably a therapeutically effective amount), and/or a salt, solvate, hydrate or prodrug thereof, is administered to treat a subject with [or suspected to have, or at risk of] cancer (or to treat a subject with a different disease/disorder/condition [including aging] mentioned herein, i.e. which can be treated with an F₁F₀ ATP hydrolysis inhibitor compound). In alternative embodiments, an amount of a racemate or scalemate of 9a and 9b (preferably a therapeutically effective amount), and/or a salt, solvate, hydrate or prodrug thereof, is administered to treat a subject with [or suspected to have, or at risk of] cancer (or to treat a subject with a different disease/disorder/condition [including aging] mentioned herein, i.e. which can be treated with an F₁F₀ ATP hydrolysis inhibitor compound).

The structure of 8a and 8b, but with trifluoromethyl (CF₃), instead of methyl (CH₃), on the chiral carbon is less susceptible to metabolism at this position, and it cannot racemize, and thence its stereoisomer preference for cancer therapy (and therapy for a different disease/disorder/condition [including aging] mentioned herein, i.e. which can be treated with an F₁F₀ ATP hydrolysis inhibitor compound) is less complicated and more defined, wherein its S stereoisomer is more favoured than its R stereoisomer. Its S stereoisomer adopts a similar structure than 6b, 7b and 8b. Its R stereoisomer adopts a similar structure to 8a (when it is unmetabolized, with CH₃ on its chiral carbon), which is actually quite structurally distinct from 6a or 7a, but which also doesn't have good inhibitory potency for F₁F₀ ATP hydrolysis.

FIG. 2: The anti-cancer activity of compounds 6a, 6b, 7a, 7b, 8a and 8b (shown in the figure) are all correlated, which suggests that they all exert anti-cancer activity by the same mechanism, inhibition of F₁F₀ ATP hydrolysis, and the bottom table shows their pairwise Pearson correlation (R) coefficients, all significant (p<0.05). In some disclosure embodiments, one or more of the compounds shown in this figure is in stereoisomeric excess such that it rotates polarized light in the levorotatory (L) direction, optionally wherein this compound(s) in stereoisomeric excess (L-rotating) is used to convey therapy in a subject, in a method of treatment of the human or animal body by therapy, optionally to treat/ameliorate/prevent/combat one or more diseases/disorders/conditions referred to in this disclosure, optionally cancer, optionally for the treatment/amelioration/prevention/combat of cancer in a subject(s), and/or for the manufacture of a medicament, optionally for treating one or more of the diseases/disorders/conditions referred to in this disclosure, optionally cancer. Without wishing to be bound by theory, the order of anti-cancer activity can be explained if the order of inhibitory potency against F₁F₀ ATP hydrolysis is: R (CH₂OH) >S (CH₂OH)≈S (Me) >S (D) >S (H) >R (Me) >R (H) >R (D). Explaining first that R (H) and S (H) cannot, and can, potently inhibit F₁F₀ ATP hydrolysis respectively [8, 109]. Starting from the bottom of the ranking: R (H) has greater anti-cancer activity than R (D) because a CH bond is weaker than a CD bond and so it has a greater chance of racemizing to S (D) or S (H). R (Me) has greater anti-cancer activity than R (H) because in some cases, over time, some/all of it is metabolized to R (CH₂OH), wherein R (Me) itself, unmetabolized, is at the bottom of the list (not shown), with worse inhibitory potency against F₁F₀ ATP hydrolysis than R (D), which has some chance of racemizing to S (D) or S (H). S (H) has greater anti-cancer activity than R (Me) because all of it is active rather than just some metabolized fraction, especially because some cancer cell lines have poor metabolism of R (Me). S (D) has greater anti-cancer activity than S (H) because a CD bond is stronger than a CH bond and so it has less chance of racemizing to R (D) or R (H). S (Me) and S (CH₂OH) have greater anti-cancer activity than S (D) and S (H) because they have no (but non-zero) chance of racemizing to R (H) or R (D). R (CH₂OH) has a structure very suited to inhibiting F₁F₀ ATP hydrolysis, very distinct from the structure of R (H), more akin to S (H), but better.

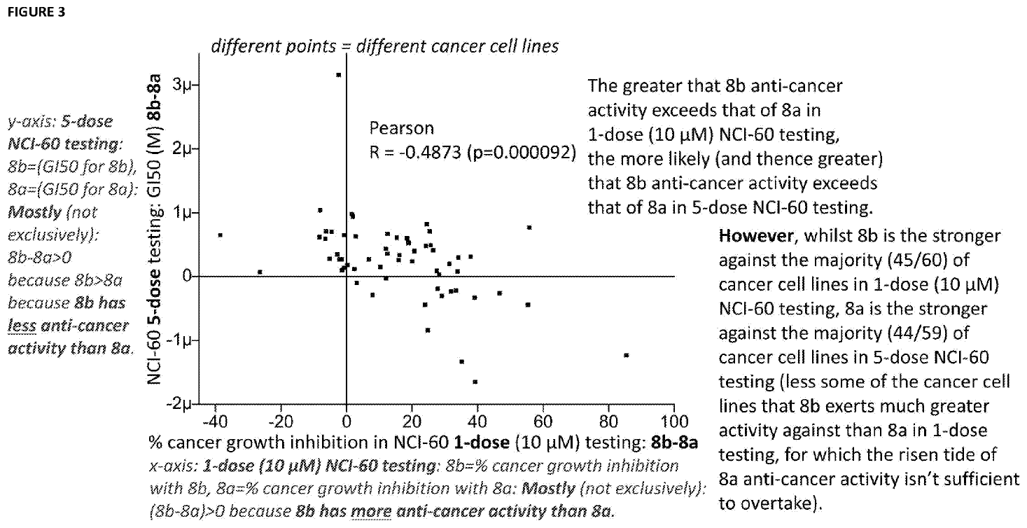

FIG. 3: FIG. 10 in Canadian application number 3,050,553 shows the anti-cancer activity of 8a and 8b in the NCI-60 five-dose in vitro assay 1107-1081. The present figure, herein, is a summary figure of that data (corresponding to FIG. 16K in the aforementioned Canadian application). Mean G150 for 8b and 8a is 3.09 and 2.85 μM respectively. So, 8a has the lower mean GI50 and so is the more potent. In the 8a case, this GI50 is lower/better than 65% of 102 FDA approved cancer drugs in [112]. A salt of 8a. e.g. 8a HCl, is likely to have an even lower G150 in NCI five-dose testing.

On average, 8b exerts greater anti-cancer activity than 8a in 1-dose (10 μM) NCI-60 testing (FIG. 1). On average, 8a exerts greater anti-cancer activity than 8b at 10 μM in 5-dose NCI-60 testing. So, the 1-dose and 5-dose results contradict. However, there hasn't been a mix up of 8a and 8b samples because in 1-dose (10 μM) testing, in FIGS. 1E and 1D, one can see that 8b exerts much greater activity against, for example, the MDA-MB-231/ATCC, OVCAR-5, HCC-2998 and NCI-H322M cell lines than 8a. In the 5-dose testing, although most GI50s are lower with 8a, the GI50 for these cancer cell lines is greater with 8a than 8b. So, this signature carries through.

In the present figure: in 1-dose (10 μM) NCI-60 testing, cancer growth inhibition (%) with 8b tends to be greater than with 8a, and 8b-8a is positive. By contrast, in 5-dose testing, GI50 tends to be lower for 8a than 8b, because 8a has the more potent anti-cancer activity, which makes G150(8b-8a) positive. The exceptions to this tend to be for cancer cell lines against which 8b exerts much greater anti-cancer activity than 8a in 1-dose NCI-60 testing, so wherein 8b-8a is large, and then in 5-dose testing, GI50 tends to be lower for 8b than 8a, which makes G150(8b-8a) negative. For these cancer cell lines, 8b has so much greater anti-cancer activity than 8a in 1-dose testing that despite the increased anti-cancer of 8a relative to 8b in 5-dose testing, it isn't sufficient to overtake that of 8b in these cases.

The Pearson correlation coefficient between 8a activity in 1-dose (10 μM) and at 10 μM in 5-dose NCI-60 testing: R=0.4544 p=0.00034. The Pearson correlation coefficient between 8b activity in 1-dose (10 μM) and at 10 μM in 5-dose NCI-60 testing: R=0.6156, p<0.00001. In 5-dose NCI-60 testing, at 10 μM, the anti-cancer activity of 8a and 8b is greater than at 10 μM in 1-dose NCI-60 testing. Mean % cancer growth inhibition at 10 μM in 1-dose testing: 8a (60.32%), 8b (76.51%); in 5-dose testing: 8a (102.97%), 8b (99.42%). Median % cancer growth inhibition at 10 μM in 1-dose testing: 8a (59.83%), 8b (76.99%); in 5-dose testing: 8a (95%), 8b (93%). A compound exerting greater anti-cancer activity at 10 ppm in 5-dose than 1-dose NCI-60 testing is typically what is observed for compounds in the DTP database 11131. So, this isn't unexpected. Not wishing to be bound by theory, in 5-dose NCI-60 testing, why 8a has a lower mean GI50 than 8b will now be explained. 8a does exert greater anti-cancer activity than 8b against some cancer cell lines in 1-dose (10 μM) NCI-60 testing (FIGS. 1D and 1E). As explained in the legend of FIG. 1, because it can be metabolized to a form (hydroxylated on the chiral carbon) with greater anti-cancer activity than 8b. In 5-dose NCI-60 testing, whatever the reason(s) that compounds tend to exert greater anti-cancer activity at 10 μM in 5-dose than 1-dose NCI-60 testing, perhaps (to speculate) relating to greater compound availability because better care/optimization is taken over compound solubilizing, means there is more 8a, so more 8a being metabolized to the more active form (increased substrate, increased reaction rate and product), and this tips 8a into exerting greater anti-cancer activity at 10 μM than 8b. Except for the cancer cell lines that 8b exerted much more activity than 8a at in 1-dose testing, wherein the increased activity of 8a can't completely make up the deficit and overtake the activity of 8b.

### If1 Protein Activity is a Molecular Determinant of Lifespan

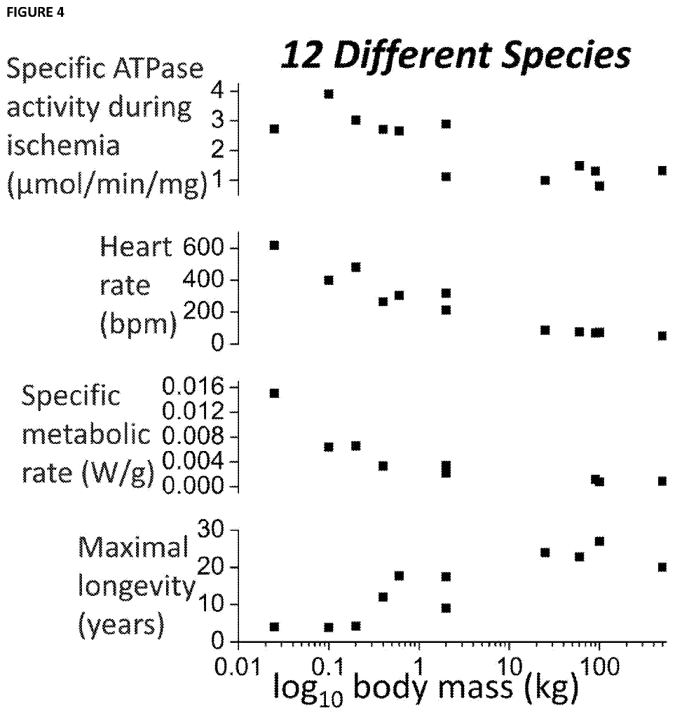

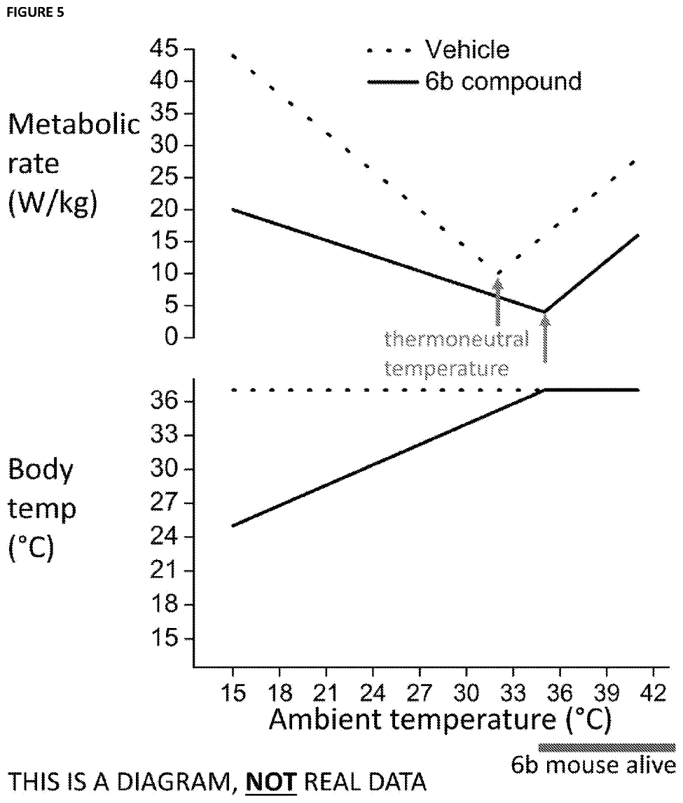

FIG. 4: Data teaching that IF1 protein activity is a molecular determinant of lifespan, therein explaining why different species have different maximal lifespans.

How can a dog be younger chronologically, but older biologically, than its human owner?Why do dogs age faster than humans? Why do different species age at different rates and have different maximal lifespans? This disclosure answers these questions. Presenting data in this figure that shows that IF1 protein activity is a molecular determinant of lifespan, therein explaining why different species have different maximal lifespans.

Smaller species tend to have shorter lifespans than larger species. Indeed, as observed in the figure, smaller species have a greater metabolic rate per unit mass, faster heartbeat and shorter lifespan than larger species. So, it seems that smaller species live fast, die young. Whilst larger species live slower for longer. Herein I disclose a reason/mechanism for this and how to manipulate it.

Figure: data in upper two figure panels is from [114], data in lower two panels is from the AnAge database [115]. Interpretation here is novel. There was some margin for error in marrying these two data sets because [114] uses imprecise terms such as sheep, hamster etc. wherein there are a number of different species in [115] that can fall into these categories. But a common sense alignment was applied in each case, by estimating which species [114] likely had easiest access to, so most likely used, and so most likely refer to. So, the 12 species that the present figure refers to are: cow (domestic cattle, Bos taurus), mouse (house mouse, Mus musculus), rat (Rattus rattus), hamster (golden hamster, Mesocricetus auratus), guinea pig (Cavia porcellus), pigeon (common wood-pigeon, Columba palumbus), chicken (red junglefowl, Gallus gallus), rabbit (European rabbit, Oryctolagus cuniculus), sheep (domestic sheep, Ovis aries), pig (wild boar, Sus scrofa), dog (Canis familiaris) and human (Homo sapiens). All warm blooded. Specific metabolic rate data wasn't available in [115] for all these species, thence the smaller number of data points in the 3^{rd} figure panel. The maximum longevity of human from [115] (122.5 years) isn't shown in the figure because arguably this value isn't fairly comparable to the others shown, because modern medicine is disproportionally applied to humans and the verifiable longevity data set for humans is much, much bigger with so many countries recording births and deaths (the bigger the data set the greater the chance a higher maximum longevity will be found). Human could perhaps be more comparably incorporated by using a maximum longevity record from a small human data set, to mirror the small data sets for the other species, wherein this data set comes from humans living in the past e.g. from 1881 Germany where life expectancy of men and women was 35.6 and 38.5 years respectively (Statistisches Bundesamt Deutschland, www.destatis.de). However, omission was chosen instead. For domestic cattle (mass=500 kg), maximum longevity of 20 years is used in my figure, which is from [115], but [115] do caution that this value is of “questionable” quality and say in this entry's “observations” section that the real value is likely to be higher, which if applied, would make the positive trend shown here, between body mass and lifespan, stronger.

The 1^{st} figure panel shows a negative correlation between species size and mass specific F₁F₀ ATP hydrolysis during ischemia. The 3^{rd} figure panel shows a negative correlation between species size and mass specific metabolic rate. The 2^{nd} figure panel shows a negative correlation between species size and heart rate, wherein bpm refers to beats per minute. The 4^{th} figure panel shows a positive correlation between species size and maximum longevity (maximal recorded lifespan). Herein disclosed, the 1^{st} panel correlation drives the 3^{rd} panel correlation which drives the 2^{nd} panel correlation which drives the 4^{th} panel correlation.

The following table presents the Pearson correlation (R) coefficients, with associated p-values (one-tailed) below, for the different relationships:

| Pearson R correlation Coefficient | Heart rate | Specific metabolic rate | Maximal longevity |
|---|---|---|---|
| Specific ATPase during ischemia | 0.8007 | 0.5331 | −0.7194 |
| | Heart rate | 0.9034 | −0.8567 |
| | | Specific metabolic rate | −0.7472 |
| P-value (one-tailed) | Heart rate | Specific metabolic rate | Maximal longevity |
| Specific ATPase during ischemia | 0.0008765 | 0.0697165 | 0.0062885 |
| | Heart rate | 0.000419 | 0.0003785 |
| | | Specific metabolic rate | 0.0165595 |

Asymptotically exact harmonic mean p-value for combining independent/dependent tests
Of all the p-values above = 0.0009447668 = 0.0009
Fisher's combined probability test
Using p-values for [Specific ATPase during ischemia vs. Maximal longevity] (=0.0062885) &
[Heart rate vs. Specific metabolic rate] (=0.000419) = 0.000036

The p-values are small, despite the small values of n (wherein Specific metabolic rate has an especially small n value, because I only had such data for 9 of the 12 species), in testament to the high R values. The asymptotically exact harmonic mean p-value was calculated according to the method of [116] (its corrected method according to: “Correction for Wilson, The harmonic mean p-value for combining dependent tests—Oct. 7, 2019”). This value is significant using either the p-value<0.05, or the more stringent <0.01, cut-off of the art. Its value indicates that there is a 0.09% probability that the observed (or more significant) correlations could have occurred by random sampling error (i.e. the sample doesn't fairly reflect the population) if the null hypothesis is true. Note that one-tailed p-values were used because the alternative hypothesis is directional (and thence the null hypothesis encompasses the anti-directional correlations and non-correlations) i.e. it doesn't merely hypothesize a correlation in either direction for each, but a correlation in a specific direction (+ or −) for each.

IF1 protein inhibits F₁F₀ ATP hydrolysis. IF1 protein inhibits F₁F₀ ATP hydrolysis more during ischemia but its inhibition of F₁F₀ ATP hydrolysis is non-zero under normal conditions. Larger species inhibit specific F₁F₀ ATP hydrolysis more than smaller species during ischemia (1^{st} figure panel). This is because larger species have greater IF1 protein abundance, indeed a greater IF1/F₁ protein ratio, and/or because their IF1 protein has greater inhibitory potency against F₁F₀ ATP hydrolysis [114, 115, 117-127]. Thence there is a positive correlation between species size and their capability to inhibit their F₁F₀ ATP hydrolysis i.e. the bigger the species, the less F₁F₀ ATP hydrolysis.

Animal mass is proportional to animal radius³, animal surface area is proportional to animal radius² [128]. So, smaller animals have a larger surface area to mass ratio and so lose a greater proportion of their heat to the environment and so need to generate more heat per unit mass than larger animals, which they do by a greater metabolic rate per unit mass, which they achieve by greater specific F₁F₀ ATP hydrolysis, which consumes more ATP per unit mass/time, which requires more ATP be created per unit mass/time, and thence a greater metabolic rate, thence a greater specific heat production. So, smaller species have greater specific F₁F₀ ATP hydrolysis capacity than larger species (1^{st} figure panel). That F₁F₀ ATP hydrolysis is used for heat production by animals is shown by greater F₁F₀ ATP hydrolysis capability, because of greater F₁F₀ ATP synthase and lesser IF1 protein abundance, in mitochondria sourced from cows in winter than summer, wherein such seasonal changes don't occur in laboratory rats kept at constant temperature [117]. Furthermore, specifically inhibiting F₁F₀ ATP hydrolysis in mice reduces their heat generation and body temperature. Because smaller species have a higher specific metabolic rate than larger animals, more fuel/waste is needed/ejected per unit mass per unit time, and they require and have a faster heart rate (2^{nd} figure panel). Smaller species have a greater specific metabolic rate (3_{rd} panel), faster heart rate (2^{nd} panel) and lower lifespan (4^{th} panel), wherein I innovatively suggest this is a function of their greater F₁F₀ ATP hydrolysis capacity (1^{st} panel) as a function of their lesser IF1 protein inhibitory capacity. And thence increasing the abundance of IF1 protein in a species, and/or expressing/administering the IF1 protein amino acid sequence of a larger and/or longer living species, increases the lifespan of a species, F exogenous heat (and/or greater body insulation) substitutes for the lower endogenous heat production that will ensue. Similarly, a specific/preferential drug inhibitor(s) of F₁F₀ ATP hydrolysis, for non-limiting example a compound(s) of Formula (I) herein, increases the lifespan of a subject, IF exogenous heat (and/or greater body insulation) substitutes for the lower endogenous heat production that will ensue. A disclosure embodiment is a method of administering a specific or preferential inhibitor(s) of F₁F₀ ATP hydrolysis, for non-limiting example a compound(s) of Formula (I-V, VII-VIII) herein, to a subject to extend their health and/or lifespan. A disclosure embodiment is a method of increasing the amount of IF1 protein in a subject to extend their health and/or lifespan. A disclosure embodiment is a method of administering a subject one or more of an IF1 protein, which has a greater inhibitory potency against F₁F₀ ATP hydrolysis than their endogenous IF1 protein, especially at pH 8, to extend their health and/or lifespan, optionally wherein one or more of a gene or polynucleotide or DNA or RNA is administered that is translated into such an IF1 protein. A disclosure embodiment is to express/administer the IF1 protein of a larger species in a smaller species to increase the health and/or lifespan of the smaller species. A disclosure embodiment is to express/administer the IF1 protein of a longer living species in a shorter living species to increase the health and/or lifespan of the latter.

So this data teaches that IF1 protein activity is a molecular determinant of lifespan, therein explaining why different species have different maximal lifespans, teaching selective F₁F₀ ATP hydrolysis inhibitors (e.g. cell-penetrating IF1 fusion proteins and functional fragments, and variants/derivatives, thereof) to extend health- and life-span. IF, when the subject has an effective amount of an exogenous/administered F₁F₀ ATP hydrolysis inhibitor compound(s) in their system, exogenous heat/temperature to the subject (and/or their body insulation e.g. by clothing) substitutes for their lower endogenous heat production, optionally wherein the administration pattern selected is such that the subject only has an effective amount of a F₁F₀ ATP hydrolysis inhibitor drug(s) in their system some of the time, e.g. when they are trying to sleep/sleeping, optionally which they do at a safe elevated/heated temperature (to their geographical location's ambient temperature at that time). An administered F₁F₀ ATP hydrolysis inhibitor drug(s) increases the thermoneutral temperature of the subject, and/or the temperature at which they feel comfortable, which can actually assist the subject, in and of itself, in a hot country, and which can be countered in a cold country by wearing more clothing and/or increasing room temperature (e.g. that in which the subject sleeps/relaxes/works/travels). When the ambient temperature to the subject is 37° C. or greater, the subject needn't endogenously generate any heat to keep their body temperature at 37° C., indeed any endogenously generated heat is then acting to move the body temperature away from the optimum temperature, and so is a curse and not a benefit. The thermoneutral/thermocomfortable temperature for a human, wearing clothes, tends to be in the range of 18 to 22° C. It requires costly air conditioning in many parts of the world, at least during summer, to set a room temperature to within this range. So, in these parts, at least during summer, there is dual benefit in using a F₁F₀ ATP hydrolysis inhibitor drug(s) to increase a human's theremoneutral/thermocomforable temperature, wherein the amount of shift is set by the dose administered, wherein there is a dual thermoregulatory and an anti-aging benefit. So, in colder climates the thermoregulatory aspect can be considered a bug that needs to be offset, but in hotter climates, the thermoregulatory aspect can be considered a feature, which actually confers benefit in and of itself: stand-alone benefit, conferring slower aging atop.

Incidentally, the newly discovered physiology of homeothermy, disclosed herein, is a very elegant system: when the body is resting and so isn't performing much work, F₁F₀ ATP hydrolysis (and its drive to F₁F₀ ATP synthesis and metabolic rate) confers endogenous heat production. But when the body is active and performing appreciable work, ATP is consumed to do this work (inherently generating heat as a by-product, 2^{nd} Law of Thermodynamics), and so there is less ATP available to F₁F₀ ATP hydrolysis, which is reduced as a result. So when performing work, which inherently generates heat, the futile (no work) process to generate heat is decreased, affording more energy to actually perform work, and reducing the chance that the body will overheat. So, in the resting state, heat generation is (fractionally) by a futile process, until more work is required, after which the heat generation by the actual performing of work substitutes for a reduction in heat generation by the futile process. Elegant. When an effective amount of a compound(s) of this disclosure, for example at least one compound of at least one of Formula (I), (II), (III), (IV), (V), (VII), (VIII), [X], and/or another compound(s) that selectively inhibits F₁F₀ ATP hydrolysis, and/or a pharmaceutically-acceptable salt, solvate, hydrate or prodrug thereof, and/or a pharmaceutical composition(s)/medicament(s)/supplement(s) thereof, is administered (and/or self-administered) to a subject whilst/before they are performing work, it reduces the amount of F₁F₀ ATP hydrolysis further, increasing the amount of ATP energy available to perform work, increasing physical and/or mental performance/endurance, wherein the additional ATP consumed for more work inherently generates more heat, which (partially/completely) substitutes for the further decrease in heat generation by F₁F₀ ATP hydrolysis.

When not performing ADP/ATP exchange across the mitochondrial inner membrane, the Adenine Nucleotide Translocator (ANT) can passage protons [129]. When the body is at rest, not enough cellular work is performed to generate the required heat as a by-product, and so heat needs to be produced by a futile (no work performed) process. Wherein at rest there is less ADP/ATP exchange by ANT (because of less cellular demand for ATP), and so ANT is more available to passage protons, and participate in a futile cycle with F₁F₀ ATP hydrolysis pumping protons into the mitochondrial intermembrane space, ANT (and/or other members of the SLC25 Mitochondrial Carrier Family) passing protons back into the mitochondrial matrix, and this ATP consumption pulling through more ATP synthesis by oxidative phosphorylation, all acting to generate sufficient heat to keep the body warm at rest. Wherein more heat needs to be generated per unit mass in smaller than larger animals because they have a larger surface-area (A) to volume (V) ratio, because in Euclidean geometry, A∝cr² and V∝r³ where r is radius, and so they tend to have a greater metabolic rate and so accumulate “miles on the clock” (age) faster. The amount of this futile process is controlled by IF1 protein activity, which curtails it, wherein smaller, shorter-living species tend to have less IF1 protein activity, as shown in the present figure.

Reducing F₁F₀ Hydrolysis Reduces Body Temperature

The data of FIG. 4 herein makes a prediction: administration of a compound that can inhibit/reduce F₁F₀ hydrolysis (thence reduce futile cycling of ATP synthesis and hydrolysis and its heat generation), e.g. compound 6b, to a subject will reduce their body temperature, if the ambient temperature is below their body temperature. FIG. 15 in PCT/EP2018/069175 (and FIG. 23 in Canadian application number 3,050,553) shows that compound 6b, when administered to a mouse, reduces its rectal temperature towards the ambient room temperature (22° C.), in a dose dependent manner, wherein a larger dose can result in a greater rectal temperature drop, wherein this rectal temperature drop conferred sedation/hypoactivity in the mice. The figure teaches that if Body Temperature (BT) is greater than Ambient Temperature (AT), compound 6b reduces BT to be closer to AT, but it canNOT reduce BT below AT; compound 6b can reduce BT to more nearly above AT (BT≈AT), but not completely reducing to BT=AT because there are other aspects to metabolic heat production than F₁F₀ ATP hydrolysis, which compound 6b does not reduce, and so BT remains higher than AT (BT≈AT<BT), unless the animal dies, in which case BT=AT. If AT is at or above optimal body temperature, upon compound 6b administration, BT will remain at this optimum because compound 6b canNOT reduce BT below AT. In some disclosure embodiments, when the subject has a compound(s) of this disclosure in its system, for example a compound(s) of Formula (I), the subject is kept in an ambient temperature at or near 37° C. i.e. at or near the optimal body temperature of a mammal. This ensures that the subject's body temperature cannot fall below this optimal body temperature. This renders a compound(s) of this disclosure safer and tolerable at higher dose, which can enable the compound(s) to safely convey greater therapeutic utility e.g. greater anti-cancer activity. To illustrate by analogous example, anaesthetic can dramatically reduce subject body temperature, but not when the body is kept at 37° C. ambient temperature [30]. An ambient temperature equal to optimal body temperature can keep body temperature at its optimum when a compound(s) that reduces body heat generation and/or increases body heat dissipation is administered to the subject.

FIG. 5: This is a diagram relating to mouse and does NOT present real data, although it is inspired by experimental data in [6]. When ambient equals thermoneutral temperature, which is ˜32° C. normally for a mouse [6], the mouse's basal heat production (heat production of the basal metabolic rate) is sufficient to maintain body temperature at ˜37° C. At lower ambient temperatures than this, greater metabolic rate/heat production (thermogenesis) is required, and at higher ambient temperatures than this, greater metabolic rate is required for cooling, all to maintain body temperature at ˜37° C. A specific F₁F₀ ATP hydrolysis inhibitor, e.g. compound 6b, reduces the mouse's basal metabolic rate and shifts its thermoneutral temperature higher, illustratively to 35° C. in this figure, which makes the mouse more comfortable (lower metabolic rate) at higher ambient temperatures. Furthermore, this figure anticipates that F₁F₀ ATP hydrolysis is integral to thermogenic metabolic rate, in addition to basal metabolic rate, and so the gradient of the thermogenic metabolic rate increase is shallower, because of reduced F₁F₀ ATP hydrolysis, and thus the mouse is unable to maintain 37° C. body temperature at lower ambient temperatures than its thermoneutral temperature. The metabolic rate at thermoneutral temperature=35° C. was selected by drawing a line from metabolic rate at thermoneutral temperature=32° C., =10 W/Kg, which is an experimental data point from classic Herrington albino mouse study described in [6], to 37° C. on the x-axis (thermoneutral temperature=37° C., metabolic rate=0 W/kg) and selecting the corresponding metabolic rate for 35° C. on this line. Accordingly the basal metabolic rate was 60% lower and, in accordance, the gradient of the thermogenic plot was reduced by 60% also, anticipating that F₁F₀ ATP hydrolysis contributes equally to basal and thermogenic metabolic rates, although it probably contributes more to the thermogenic than basal metabolic rate, in which case the ascending thermogenic metabolic gradient can be shallower, and the descending body temperature gradient steeper, than shown (although it cannot outpace the gradient of ambient temperature decline: the mouse body temperature must be higher or at {if metabolism=0 W/kg} ambient temperature). Conclusion: mice administered with 6b compound cannot survive at as low temperatures than vehicle treated mice. However, if kept at higher temperatures, at or safely greater than their thermoneutral temperature, the lower metabolic rate of 6b administered mice confers them longer lifespan. When the connection between work (unit: joule) and time is disrupted, the simple correlation between age and time is broken. Oxidative metabolism produces damaging/aging Reactive Oxygen Species (ROS) and a lower oxidative metabolic rate produces less ROS per unit time, reducing the damage/aging rate, extending lifespan. An older (in time) 6b administered body can be younger (in aging/damage) than a younger (in time) vehicle administered body. Like a car, the less miles per unit time it drives, the longer it lasts, like a body metabolism, the less oxidative work (joules) it performs per unit time, the longer it lasts (P.S. health benefit of exercise doesn't breach this: exercise provokes adaptive changes that means the body works less at rest, e.g. endurance athletes have lower resting heart rate, =net reduction in work performed by body).

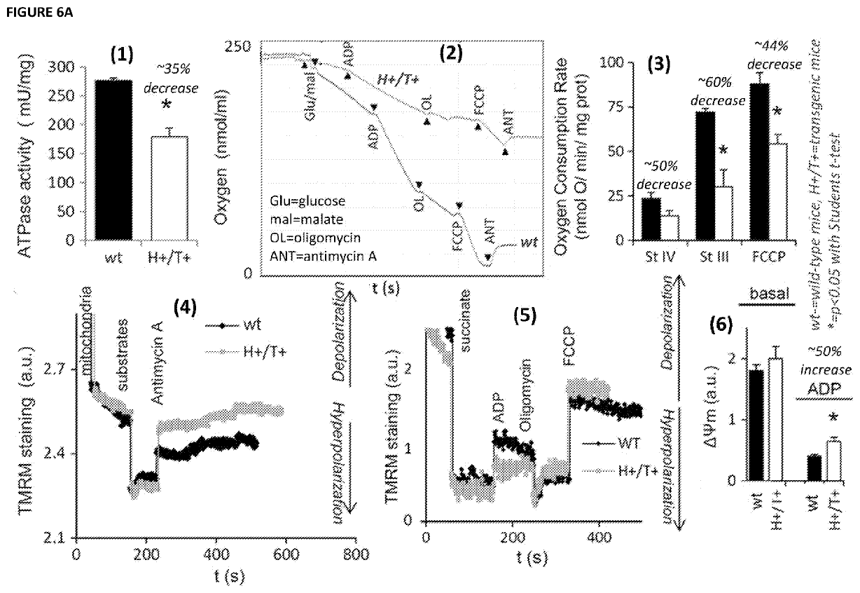

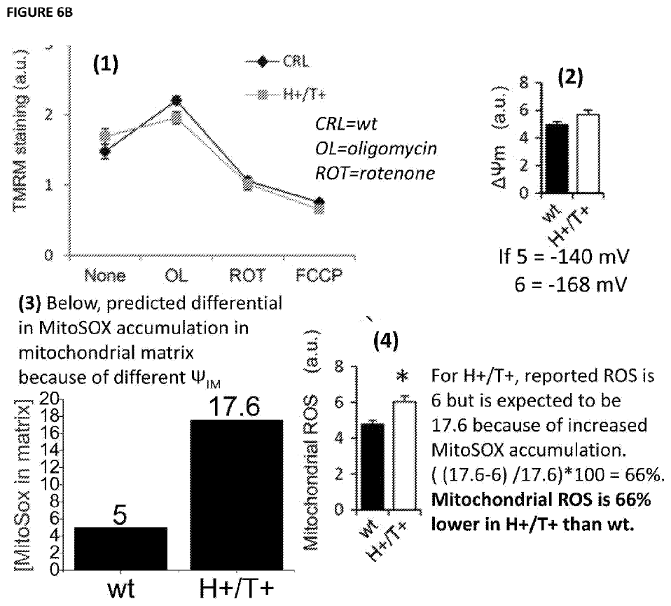

FIG. 6: In vivo, inhibiting F₁F₀ ATP hydrolysis safely reduces the rate of oxidative phosphorylation and ROS generation {and thence the rate of aging}. Shown in forebrain neurons. This figure presents re-interpreted data from [130].

[130] is an illustrative example of how an IF1 gene copy, or a mutant thereof, from the same or different species, can be transferred into an organism in order to increase its IF1 protein expression. This example shows it is safe in mouse brain (more specifically neurons in forebrain) to increase IF1 protein content by three times (300%), wherein the delta increase in [130] occurs with a mutant human IF1 protein form with increased inhibitory potency against F₁F₀ ATP hydrolysis at pH 8, which is observed to reduce F₁F₀ ATP hydrolysis capability by ˜35%, which demonstrates the safety of inhibiting F₁F₀ ATP hydrolysis in vivo, at least specifically in forebrain neurons (mice were “normal in appearance, home-cage behaviour, reproduction, and longevity up to 1-year follow-up”).

The relevance of the following will become apparent later, wherein my analysis shows that MitoSOX™, an ROS (superoxide) reporting fluorogenic dye, accumulates more in the mitochondrial matrix than Tetramethylrhodamine Methyl Ester Perchlorate (TMRM), a fluorescent dye used to report Ψ_{IM}:

```
Basic pKa (MACRO, static, T = 310K) computationally predicted from
structure using MarvinSketch (ChemAxon)

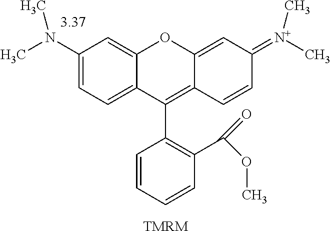

Double positively charged
@pH 6.8 = 0.04%; @PH 8 = 0%.
[matrix] = (99.96*10\hat{ }(−140/−61.5)) +
(0.04*10\hat{ }(−140/−46.1)) = 18,934

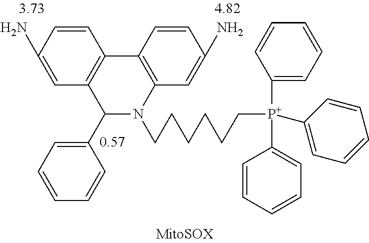 [SMILES: Nc1ccc2c(c1)C(c1ccccc1)N(CCCCCC[P+](c1ccccc1)(c1ccccc1)c1ccccc1)c1cc(N)ccc1-2]

Double positively charged
@pH 6.8 = 1.03%; @pH 8 = 0.07%.
[matrix] = (98.97*10\hat{ }(−140/−61.5) +
(0.96*10\hat{ }(−140/−46.1)) +
(0.07*10\hat{ }(-140/-30.7)) = 22,292
Disparity between TMRM and MitoSOX
accumulation in matrix increases with ψ_{IM}
Ratio = 22,292/18,934 = 1.18 (ψ_{IM} = −140 mV)
                    = 1.34 (ψ_{IM} = −160 mV)
                    = 1.68 (ψ_{IM} = −180 mV)
                    = 2.38 (ψ_{IM} = −200 mV)
$Nernst equation : [ matrix ] = [ IMS ] \cdot {10}^{( \frac{{Ψ}_{IM}}{- G} )}$
G = 1000*2.3026*(RT/zF);
where 1000 is a dimensionality factor to work in mV,
R = gas constant (8.31 J · mol⁻¹), T = temperature (K), z = species charge,
F = Faraday constant (96,485 C · mol⁻¹). G = 61.5 (cation), 30.7
(di-cation),
−61.5 (anion), −30.7 (di-anion) at T = 310K = 37° C.
Use G = (61.5 + 30.7)/2 = 46.1 for a species that is +2 on one side,
and +1 on other side, of membrane.
matrix = mitochondrial matrix, IMS = mitochondrial intermembrane
space, IM = mitochondrial inner membrane,
ψ_{IM} = voltage across IM = −140 mV, pH of IMS = 6.8 (but might be as
low as 6), pH of matrix = 8.
```

(FIG. 6A) Experiments with mitochondria extracted from the brain of wild-type mice (wt) and double transgenic mice (H+/T+) with: (i) a mutant human IF1 protein gene (with a H49K substitution, i.e. with a histidine [H] in its “pH dependence motif” {FIG. 10}substituted with lysine [K]) under a tetracycline-responsive promoter element (TRE), and (ii) a tetracycline-controlled transactivator protein gene (tTA) under the control of the CaMKIIα promoter, wherein CaMKIIα is only expressed in forebrain neurons [131], thence tTA and thence the human H49K IF1 protein gene is only expressed in the mouse's forebrain neurons (in the absence of a tetracycline(s) e.g. doxycycline), wherein their IF1 protein amount (native+mutant) is 3 times (i.e. 300%) greater than wild-type. The H49K substitution renders an IF1 protein with greater inhibitory potency against F₁F₀ ATP hydrolysis at normal mitochondrial matrix pH (8). F₁F₀ ATP hydrolysis is 35% less in brain mitochondria isolated from H+/T+ than wild-type mice. The reduction in F₁F₀ ATP hydrolysis doesn't match the increase in IF1 protein. Perhaps because an IF1 protein fraction is inactivated by phosphorylation on its “phosphorylation control switch” serine residue (FIG. 10). Wherein in some disclosure embodiments, IF1 protein is used with this serine substituted for another residue, optionally alanine, so that it cannot be phosphorylated at this position and thence cannot thereby be inactivated; and in further embodiments, this IF1 protein also has a H49K substitution. H+/T+ mice have a lower respiration (O₂ consumption, including oligomycin sensitive O₂ consumption) rate than wild-type, during State 4 (substrate [e.g. glucose, malate] stimulated) and State 3 (+ADP stimulated) respiration. Probably as a function of their lower respiration rate, H+/T+ mice have a more hyperpolarized membrane potential across their mitochondrial inner membrane, Ψₘ (also referred to as Ψ_{IM} herein), because not so much of their proton motive force (pmf) is being eroded per unit time to drive ATP synthesis. However, upon FCCP/antimycin A administration, H+/T+ mice have a more depolarized Ψ_{IM} than wild-type because the response to an uncoupler/respiratory chain inhibitor involves global reversal of ATP synthase and F₁F₀ ATP hydrolysis to pump protons, partially maintaining Ψ_{IM}, wherein F₁F₀ ATP hydrolysis capability is partially compromised in H+/T+ mice.

(FIG. 6B) Experiments with cortical neurons in culture, after being cultured for 9-10 days, after being extracted from mouse embryos. The 1^{st} and 2^{nd} panel shows disparity in Ψ_{IM} between H+/T+ and wild-type (CRL) mice again. As in FIG. 6A. But this time in cultured cortical neurons from the mice (embryos), wherein the mitochondria of H+/T+ mice have a more hyperpolarized Ψ_{IM} (accumulate more TMRM⁺) than wild-type. But, as in FIG. 6A, have a more depolarized Ψ_{IM} upon FCCP/respiratory chain inhibitor (antimycin A/rotenone) administration. A typical value for Ψ_{IM} in normal mitochondria is −140 mV and if we equate the 5 a.u. value for wild-type in the 2^{nd} panel with −140 mV, then the 6 a.u. value of H+/T+here in the 2^{nd} panel is −168 mV. This Ψ_{IM} disparity means that H+/T+ mitochondria accumulates more MitoSOX ROS (superoxide) reporting compound in their mitochondrial matrix, wherein this disparity can be calculated using the equation presented earlier, and this disparity is shown here, in the 3^{rd} panel. With this MitoSOX accumulation disparity, one would expect a greater MitoSOX signal from H+/T+ mitochondria, to wild-type mitochondria, in the proportion shown in the 3^{rd} panel. But what is actually experimentally observed is in the 4^{th} panel. Thence H+/T+ mitochondria must produce 66% less ROS (superoxide) than wild-type, which fits with their lower oxidative respiration rate observed in (FIG. 6A). Thus, given that ROS are the drive to aging [132, 133], H+/T+ cells have a slower aging rate than wild-type. Indeed, the reduced ROS in the forebrain neurons of H+/T+ mice is likely underestimated by this in vitro cultured neuron assay. H+/T+ neurons have less O₂ consumption, as shown in (FIG. 6A), and so in neuron culture, because of this lesser O₂ consumption, H+/T+ neurons experience greater pO₂ near their respiratory chain, which favours increased [ROS], wherein this is an experimental artefact because in vivo reduced O₂ consumption doesn't increase pO₂, because breathing (rate, depth etc.) maintains tissue pO₂ within a narrow range.

[130] interpret the lesser O₂ consumption of H+/T+ mitochondria as evidence that IF1 protein directly inhibits F₁F₀ ATP synthesis (which suggests no clinical utility because F₁F₀ ATP synthesis is essential to aerobic life). This is incorrect. As elucidated by the work of this disclosure, substantial F₁F₀ ATP hydrolysis is occurring under normal conditions in mice, which sets OXPHOS at high rate, to generate heat. Increased [IF1 protein] inhibits F₁F₀ ATP hydrolysis more, and so less ATP needs to be made by F₁F₀ ATP synthesis, thence less OXPHOS is required, less O₂ is consumed (when F₁F₀ ATP hydrolysis capability is reduced by ˜35% there is ˜60% less O₂ consumption during State 3 respiration), thus less ROS are generated per unit time, thence aging is slower: ˜60% slower during State 3 respiration, by the teaching of this disclosure, reinterpreting data of 11301. Less heat is produced, but because in this case this effect is limited to neurons of the forebrain, heat transfer from other mouse body regions, and from astrocytes in the forebrain (which don't express CaMKIIα, so don't express tTA, so don't express the transgenic IF1 gene), maintains an appropriate temperature in forebrain neurons. By the teaching of this disclosure, the H+/T+ mice are disclosed to have slower aging in forebrain neurons, wherein they have reduced [susceptibility to/progression of] brain diseases of aging e.g. neurodegenerative diseases like Alzheimer's disease, dementia, Parkinson's disease etc., and less cognitive decline with aging (e.g. as assayed by one or more of the mouse behavioural assays disclosed elsewhere herein, or another “brainspan”/cognitive assay known to one of the art). This feature can be stopped by administering tetracycline/doxycycline to these mice, which blocks IF1 transgene expression in their forebrain neurons. By contrast to the present disclosure, [130] claim that this mutant IF1 gene introduction increases ROS and oxidative stress in mouse forebrain neurons. Given the linkage between oxidative stress and the development/progression of neurodegenerative diseases [24], [130] teaches that these transgenic mice have increased susceptibility to/progression of neurodegenerative disease(s) than wild-type mice.

To briefly discuss some of the data in [130], that isn't replicated in this figure, but that is also re-interpreted herein. The protein carbonylation assay that [130] uses can be unreliable (“numerous problems with data reproducibility or production of spurious results” [134]) and/or, furthermore is corrupted by the large difference in [tubulin] between the H+/T+ and wild-type mice (refer the western blots; [tubulin] is used as a denominator in their carbonylation calculation). Brain of H+/T+ mice has less [ATP], but less [ADP] also, and so the ATP/ADP ratio isn't changed. There is greater [AMP]. These H+/T+ mice have less ATP and ADP because they have less cycling of F₁F₀ ATP synthesis (ATP generation) and F₁F₀ ATP hydrolysis (ADP generation), so a greater proportion of their nucleotide exists as AMP. This, greater [AMP], activates the AMP-activated protein kinase (AMPK; more phosphorylated active AMPK is observed [130]), which upregulates glycolysis, wherein the extra NADH it produces isn't very thermodynamically inclined to enter an electron into the respiratory chain because of the hyperpolarised Ψ_{IM} (no “sink drive”), which comes from a reduced rate of [pmf erosion (F₁F₀ ATP synthesis) to lesser pmf creation (F₁F₀ ATP hydrolysis) to generate heat]. More thermodynamically favourable is for lactate dehydrogenase to use this extra NADH, switching pyruvate to lactate, which is then exported, exporting this chemical energy to be accepted by a cell more in need of energy, elsewhere in the subject.

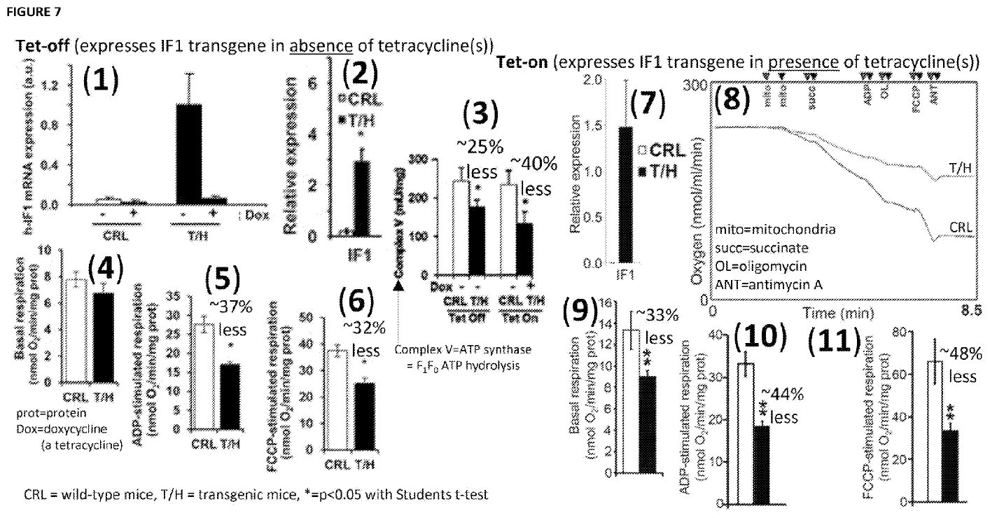

FIG. 7: In vivo, inhibiting F₁F₀ ATP hydrolysis safely reduces the rate of oxidative phosphorylation {and thence ROS generation {by extrapolation from data of FIG. 6}, and thence the rate of aging}. Shown in liver cells (hepatocytes). This figure presents re-interpreted data from [135].

[135] is an illustrative example of how an IF1 gene copy, or a mutant thereof, from the same or different species, can be safely transferred into an organism in order to increase its IF1 protein expression. Shown in this figure is data from experiments with mitochondria extracted from the brain of wild-type mice (CRL) and double transgenic mice (H/T) with: (i) a mutant human IF1 protein gene (with a H49K substitution, i.e. with a histidine [H] in its “pH dependence motif” {FIG. 10} substituted with lysine [K]) under a tetracycline-responsive promoter element (TRE), and (ii) a tetracycline-controlled transactivator protein gene (tTA) under the control of the rat liver-enriched activator protein (LAP; member of Cebpb gene family) promoter, wherein LAP is only expressed in liver cells, thence tTA and thence the human H49K IF1 protein gene is only expressed in the mouse's liver cells (in the absence of a tetracycline(s) e.g. doxycycline). In perivenous hepatocytes especially. These are “Tet-off” mice, expressing the transgenic IF1 protein gene (h-IF1) in the absence of a tetracycline e.g. doxycycline (Dox), as shown in the 1^{st} panel, wherein the 2^{nd} panel shows the presence of h-IF1 in the mouse liver cells using an antibody specific for human over mouse IF1 protein, wherein this added IF1 protein inhibits the F₁F₀ ATP hydrolysis capability by 25% (3^{rd} panel) and decreases State 3 respiration rate by 37%. Alternative transgenic mice were also generated, “Tet-on” mice, which have rtTA instead of tTA under the control of the LAP promoter, which only express the IF1 transgene in the presence of a tetracycline e.g. doxycycline (Dox), wherein this added IF1 protein inhibits the F₁F₀ ATP hydrolysis capability by 40% (3^{rd} panel) and decreases State 3 respiration rate by 44%. These experiments demonstrate the safety of inhibiting F₁F₀ ATP hydrolysis in vivo, at least specifically in liver (these transgenic IF1 mice had “no differences in weight, life span and cage behavior when compared to controls after one year of follow up”).

FIG. 8: In vivo, inhibiting F₁F₀ ATP hydrolysis safely reduces the rate of oxidative phosphorylation (and thence ROS generation {by extrapolation from data of FIG. 61, and thence the rate of aging}. Shown in intestine. This figure presents re-interpreted data from [136].

[136] is an illustrative example of how an IF1 gene copy, from the same or different species, can be safely transferred into an organism in order to increase its IF1 protein expression. Shown in this figure is data from mitochondria extracted from the colon of wild-type mice (CL), and double transgenic mice (I/T; “Tet-on”) with (i) a Non-mutant human IF1 protein gene under a tetracycline-responsive promoter element (TRE), and (ii) the intestine-specific Villin-rtTA2-M2 transactivator, wherein the human IF1 protein gene is only expressed in the mouse's intestine cells (in the presence of a tetracycline(s) e.g. doxycycline). This extra (human) IF1 protein inhibits the F₁F₀ ATP hydrolysis capability by 35% and decreases oligomycin sensitive respiration rate by 60%. This experiment demonstrates the safety of inhibiting F₁F₀ ATP hydrolysis in vivo, at least specifically in intestine.

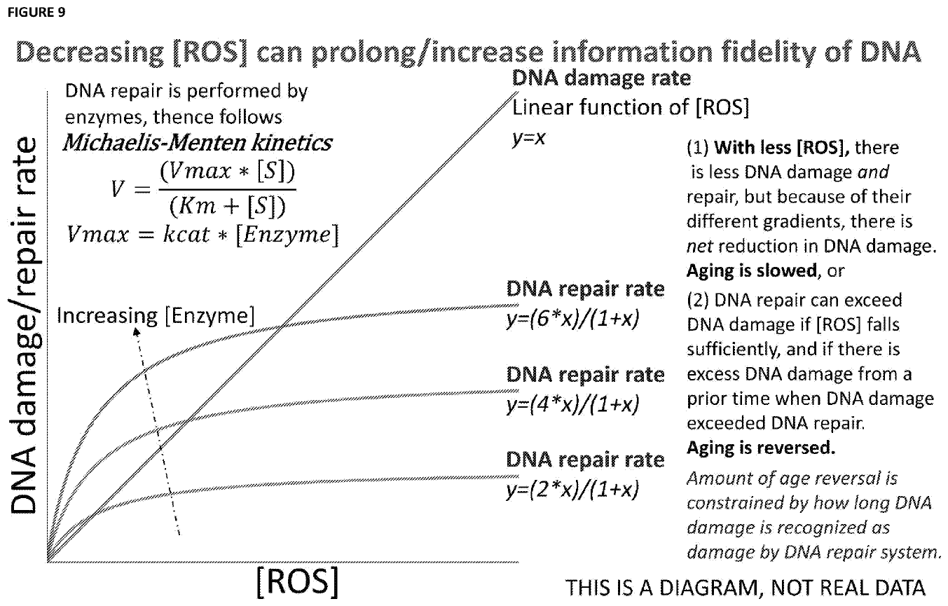

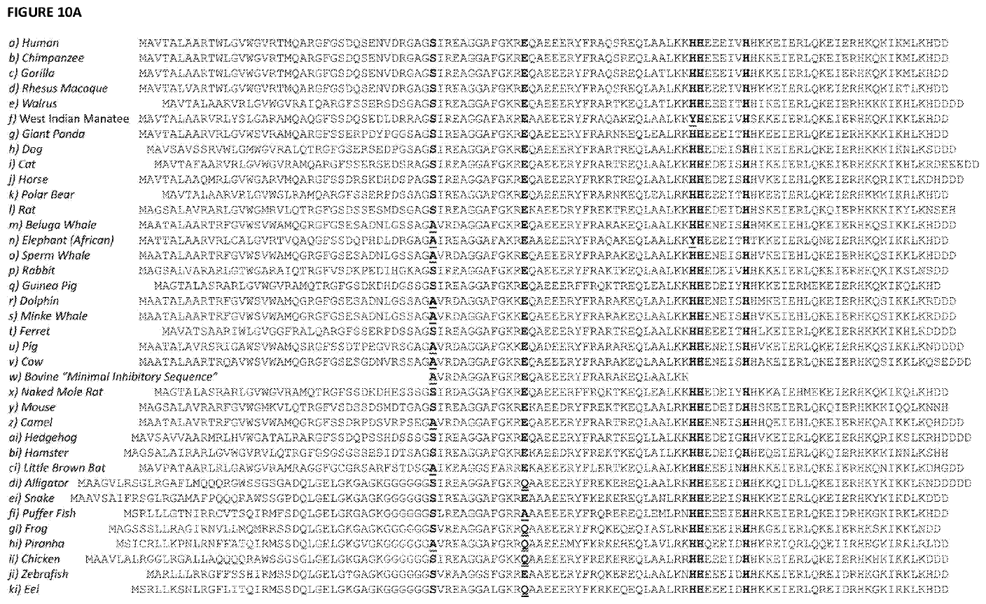

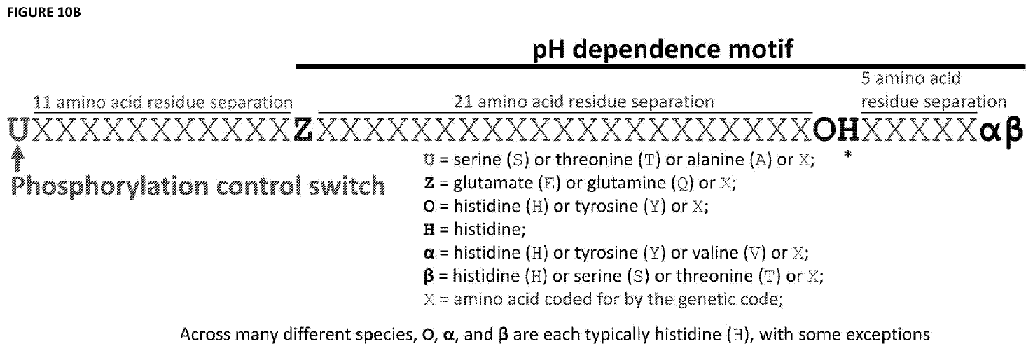

FIG. 9: Diagram (not real data) illustrating how reducing [ROS] in a cell, for example by inhibiting F₁F₀ ATP hydrolysis which reduces the oxidative phosphorylation/ROS generation rate, can prolong/increase the information fidelity of genomic/mitochondrial DNA, which slows/reverses aging. ROS=Reactive Oxygen Species. The terms in the Michaelis-Menten equation are extremely well known to those of the art. FIG. 6 shows the mechanism, (inhibiting F₁F₀ ATP hydrolysis which reduces the oxidative phosphorylation rate) and safety of reducing ROS in vivo. FIGS. 7 and 8 provide further proof of mechanism and safety. FIG. 4 elucidates that a different rate of F₁F₀ ATP hydrolysis is why different species have different maximal lifespans. Any compound(s) administered and/or method(s) that reduces/inhibits F₁F₀ ATP hydrolysis to slow/reverse aging, and/or extend lifespan/healthspan, in a subject is componentry to this disclosure, optionally wherein the expression/amount/activity of one or more DNA repair enzymes is increased in the subject also. For (non-limiting) example, any such use of a compound(s) of Formula I-V and/or VII-VIII, or a pharmaceutically-acceptable salt, solvate, hydrate or prodrug thereof. In some embodiments, a compound(s) of this disclosure, a F₁F₀ ATP hydrolysis inhibitor(s), or a pharmaceutically-acceptable salt, solvate, hydrate or prodrug thereof, is taken/administered before/during sleep, optionally wherein the subject sleeps in a temperature controlled/heated atmosphere, and/or where the subject is heated by radiative heating, optionally wherein exogenous heat substitutes for lower endogenous heat production by the subject (lower because of a compound(s) of this disclosure in their system), and wherein lesser ROS production results, thence less DNA damage per unit time, optionally wherein the rate of DNA repair thence exceeds the rate of DNA damage and so there is net DNA repair, wherein most DNA damage can still be recognised as damage (thence the possibility of being repaired) within a 24 hour period, which is a factor that permits the restorative action of sleep itself, which has a metabolism slowing/body temperature dropping (ROS reducing) component, which a compound(s) of this disclosure increases/improves. In this way, the subject doesn't need to live in a temperature controlled environment whilst awake, just when they are sleeping, and/or during some other time(s) of their choosing. In some embodiments the subject, with a compound(s) or a pharmaceutically-acceptable salt, solvate, hydrate or prodrug thereof in their system, wears heat generating/retaining clothing/device(s), optionally that monitors the subject's body temperature and adjusts its heat generating/retaining ability to maintain the subject's body at or near a desired body temperature (optionally at or near 37° C.).

In a disclosure embodiment, one or more administered F₁F₀ ATP hydrolysis inhibitors of this disclosure, or a pharmaceutically-acceptable salt, solvate, hydrate or prodrug thereof, reduces the ROS generation per unit time in a subject, which reduces their DNA damage/aging rate, such that it becomes lower than their DNA repair rate, and so their DNA (and other) repair mechanisms are under rather than overwhelmed, and their aging is stopped (repair matches damage rate) or reverses (greater repair than damage rate) so the subject becomes biologically younger, rather than older, in chronological time.

FIG. 10: Some sequence embodiments: SEQ ID NO:639 to SEQ ID NO:1425, wherein any fragment thereof (non-limiting e.g. if sequence has N-terminal Mitochondrial Import Sequence (MIS), in alternative sequence embodiments it is absent), and concatenated fragments thereof, are contemplated (as is use thereof, for at least one use disclosed herein). Presented amino acid sequences that are shorter than 4 amino acids long are not incorporated in the Sequence Listing of this application. SEQ ID NO:130, SEQ ID NO:131, SEQ ID NO:162, SEQ ID NO:163, SEQ ID NO:442, SEQ ID NO:445 are also present in this figure. Peptide/protein sequences are disclosed using one letter amino acid code. (10A) SEQ ID NO:639 to SEQ ID NO:675. IF1 proteins, from a wide diversity of species, aligned to show the incredible conservation of the bolded residues. From left to right, the 1st bolded reside is the “phosphorylation control switch” (when phosphorylated, IF1 protein cannot inhibit F₁F₀ ATP hydrolysis) and the other 4 bolded residues are part of the “pH dependence motif” [141, 138, 139] (underlining identifies a residue that deviates from the most stereotypical consensus sequence). In other protein sequence embodiments of this disclosure, one or more of these bolded residues are replaced with another amino acid, optionally coded for by the genetic code. For example, a bolded serine(S) residue, constituting the “phosphorylation control switch”, is replaced with a residue that cannot be phosphorylated (e.g. alanine), so that the IF1 protein cannot be inactivated by phosphorylation. The four other bolded residues are part of the “pH dependence motif” and amino acid substitution at one or more of its positions, optionally with alanine, increases IF1 protein inhibition of F₁F₀ ATP hydrolysis at pH 8, the normal (non-pathological) pH of the mitochondrial matrix. Also componentry to this disclosure are fragments of the shown sequences (e.g. without the Mitochondrial Import Sequence [MIS], which for human is MAVTALAARTWLGVWGVRTMQARGF [140], SEQ ID NO:162, or with a different MIS instead) and/or with a bolded residue(s) substituted for a different amino acid, optionally coded for by the genetic code. Especially favoured is a fragment with greater inhibition of F₁F₀ ATP hydrolysis at pH 8. Contemplated is a fragment that aligns with, and corresponds to, the “minimal inhibitory sequence” of bovine IF1 protein, which is the smallest, minimal fragment of bovine IF1 protein that can inhibit F₁F₀ ATP hydrolysis [141, 142], wherein a non-limiting candidate “minimal inhibitory sequence” is shown in the figure, corresponding to bovine IF1 protein residues 14-47, wherein it may actually be shorter/longer than shown (e.g. 10-47 or 16-47 or 17-47) or different (e.g. residues 42-58, or (unlikely)₂₂-46 of bovine IF1 protein). Following codes are “primary accession numbers” in UniProtKB database, SV =Sequence Version (newer sequence versions than presented herein are also hereby contemplated): (a) Q9 UII2, Homo sapiens, SV=1, (b) H2PYG9, Pan troglodytes, SV=1, (c) G3QEV8, Gorilla gorilla gorilla, SV=1, (d) F6ZXX7, Macaca mulatta, SV=1, (e) A0A2U3VIM7, Odobenus rosmarus divergens, SV=1, (f) A0A2Y9DM04, Trichechus manatus latirostris, SV=1, (g) A9XG49, Ailuropoda melanoleuca, SV=1, (h) E2QYN4, Canis lupus familiaris, SV=1, (i) M3WIS8, Felis catus, SV=2, (j) F6ZXTO, Equus caballus, SV=1, (k) A0A384CECO, Ursus maritimus, SV=1, (1) Q03344, Rattus norvegicus, SV=2, (m) A0A2Y9LD45, Delphinapterus leucas, SV=1, (n) G3SWQ8, Loxodonta africana, SV=1, (o) A0A2Y9EF27, Physeter catodon, SV=1, (p) G1SEZ3, Oryctolagus cuniculus, SV=1, (q) A0A286Y431, Cavia porcellus, SV=1, (r) A0A2U3VOR3, Tursiops truncatus, SV=1, (s) A0A383Z6R7, Balaenoptera acutorostrata scammoni, SV=1, (t) M3YVR5, Mustela putorius furo, SV=1, (u) Q29307, Sus scrofa, SV=2, (v) P01096, Bos taurus, SV=2, (w) Bovine “minimal inhibitory sequence” [141, 142], (x) G5AP86, Heterocephalus glaber, SV=1, (y) O35143, Mus musculus, SV=2, (z) S9XNE5, Camelus ferus, SV=1, (ai) A0A1S2ZPB9, Erinaceus europaeus, SV=1, (bi) A0A1U8CVF2, Mesocricetus auratus, SV=1, (ci) GINSN7, Myotis lucifugus, SV=1, (di) A0A151PGL2, Alligator mississippiensis, SV=1, (ei) AOAOB8RSH7, Boiga irregularis, SV=1, (fi) H2TBT1, Takifugu rubripes, SV=1, (gi) F7BK26, Xenopus tropicalis, SV=1, (hi) A0A3B4D9E6, Pygocentrus nattereri, SV=1, (ii) A0A1D5PBD2, Gallus gallus, SV=2, (ji) A3KNL5, Danio rerio, SV=1, (ki) A0A0E9WGC1, Anguilla anguilla, SV=1. (10B) SEQ ID NO: 676. The “phosphorylation control switch” and “pH dependence motif” of an IF1 protein [141, 138, 139]. In some embodiments, the amino acid at the “phosphorylation control switch” is substituted with a different amino acid coded for by the genetic code, preferably one that cannot be phosphorylated, optionally alanine (A). And/or one or more of the residues of the “pH dependence motif” are substituted with a different amino acid coded for by the genetic code, optionally, without restriction, tyrosine (Y), alanine (A), lysine (K), glutamate (E), glutamine (Q), valine (V), leucine (L), isoleucine (I), wherein alanine is preferred in some embodiments. Especially preferred is lysine (K) replacing the histidine (H) marked with a *, which corresponds to a H49K (“mature” [MIS cleaved off] IF1 protein numbering) substitution in the Bos taurus IF1 protein sequence. Alternatively, arginine (R) or alanine (A) replaces histidine at this position (H49R or H49A respectively). (10C) SEQ ID NO:677 to SEQ ID NO:708. Some embodiments, which are modifications of the human IF1 protein (in alternative embodiments, not shown, the N-terminal MIS sequence [first 25 residues] is absent). By the teaching of this figure, other IF1 protein(s), from human and/or other species, can be modified at one or more equivalent amino acid sequence positions i.e. at their own “phosphorylation control switch” and/or “pH dependence motif”, wherein such modified IF1 protein sequences are componentry to the present disclosure, as are nucleotide sequences that code for them by the genetic code. Any IF1 protein, e.g. any IF1 protein sequence from InterPro family “Mitochondrial ATPase inhibitor (IPR007648)”, and/or Pfam family “IATP (PF04568)”, with one or more amino acid substitutions within its “phosphorylation control switch” and/or “pH dependence motif”, wherein these sequence elements are herein defined, is componentry to the present disclosure. In further embodiments, not shown in the figure, the substituted residue at one or more of the 5 bolded positions can be any other amino acid coded for by the genetic code, wherein nucleotide sequences that encode them by the genetic code are contemplated. (10D) SEQ ID NO:709 to SEQ ID NO: 743. Preferred is an IF1 protein with the histidine (H) marked with a * in its “pH dependence motif” (FIG. 10B) replaced with lysine (K). Shown are illustrative IF1 proteins modified at this position. Wherein their site of lysine (K) substitution is bolded. As is 4 of the 5 residues of the enduring remainder of their “pH dependence motif”, which is unmodified. And their “phosphorylation control switch” residue, which is unmodified. These protein sequences are componentry to the present disclosure as are other equivalently modified IF1 proteins, not shown, as are nucleotide sequences that code for them by the genetic code, as are their sub-sequences, such as those with their N-terminal Mitochondrial Import Sequence (MIS) absent. Any IF1 protein, e.g. any IF1 protein sequence from InterPro family “Mitochondrial ATPase inhibitor (IPR007648)” and/or Pfam family “IATP (PF04568)”, with the starred (*) histidine (FIG. 10B) of its “pH dependence motif” replaced with a lysine (K), is componentry to this disclosure. Illustrative IF1 proteins modified at this position are shown in this figure (wherein the original unmodified sequences are also componentry to this disclosure, wherein later sequence versions (SV) are also contemplated): (a) Q9UII2, Homo sapiens, SV=1, (b) Q5RFJ9, Pongo abelii, SV=1, (c) A0A2R9CQC9, Pan paniscus, SV=1, (d) A0A2J8Y5Q9, Pongo abelii, SV=1, (e) H2PYG9, Pan troglodytes, SV=1, (f) A0A2I3H1P7, Nomascus leucogenys, SV=1, (g) G3QEV8, Gorilla gorilla gorilla, SV=1, (h) A0A2K6AYV8, Macaca nemestrina, SV=1, (i) A0A2K5Y149, Mandrillus leucophaeus, SV=1, (j) A0A2K5POW3, Cercocebus atys, SV=1, (k) G7NWV6, Macaca fascicularis, SV=1, (1) A0A096NQ00, Papio anubis, SV=1, (m) F6ZXX7, Macaca mulatta, SV=1, (n) A0A0D9S814, Chlorocebus sabaeus, SV=1, (0) A0A2K6N3T3, Rhinopithecus bieti, SV=1, (p) A0A2K6Q8H8, Rhinopithecus roxellana, SV=1, (q) A0A2K5SG67, Cebus capucinus imitator, SV=1, (r) A0A2K6SEK8, Saimiri boliviensis boliviensis, SV=1, (s) A0A2K5KBI5, Colobus angolensis palliatus, SV=1, (t) A0A2K5DQW7, Aotus nancymaae, SV=1, (u) F7IA10, Callithrix jacchus, SV=1, (v) A0AOD9SDU9, Chlorocebus sabaeus, SV=1, (w) A0A1D5QRM5, Macaca mulatta, SV=1, (x) A0A1U7SXJ3, Tarsius syrichta, SV=1, (y) A0A2K6Q8J3, Rhinopithecus roxellana, SV=1, (z) A0A2K6SEL7, Saimiri boliviensis boliviensis, SV=1, (ai) A0A2K6GYY1, Propithecus coquereli, SV=1, (bi) HOX2G2, Otolemur garnettii, SV=1, (ci) A0A2Y9GJM5, Neomonachus schauinslandi, SV=1, (di) A0A2K5J921, Colobus angolensis palliatus, SV=1, (ei) A0A2U3VIM7, Odobenus rosmarus divergens, SV=1, (fi) L8Y809, Tupaia chinensis, SV=1, (gi) L5JUT0, Pteropus alecto, SV=1, (hi) A0A2Y9DM04, Trichechus manatus latirostris, SV=1, (ii) A9XG49, Ailuropoda melanoleuca, SV=1. In other embodiments, not shown, instead of a H49K substitution, there is a H49A or H49R substitution instead. (10E) SEQ ID NO:744 to SEQ ID NO:780. Further IF1 proteins modified to have a lysine (K) at the starred (*) position of their “pH dependence motif” (FIG. 10B), wherein the corresponding unmodified native IF1 proteins are also contemplated (later sequence versions (SV) than shown are also contemplated): (a) E2QYN4, Canis lupus familiaris, SV=1, (b) M3WIS8, Felis catus, SV=2, (c) F6ZXTO, Equus caballus, SV=1, (d) HOXQ94, Otolemur garnettii, SV=1, (e) A0A384CECO, Ursus maritimus, SV=1, (f) D2GWK3, Ailuropoda melanoleuca, SV=1, (g) I3N8E6, Ictidomys tridecemlineatus, SV=1, (h) A0A2U3YF49, Leptonychotes weddellii, SV=1, (i) Q03344, Rattus norvegicus, SV=2, (j) A0A341D6Q8, Neophocaena asiaeorientalis asiaeorientalis, SV=1, (k) A0A2Y9LD45, Delphinapterus leucas, SV=1, (1) G3SWQ8, Loxodonta africana, SV=1, (m) A0A2Y9EF27, Physeter catodon, SV=1, (n) G1SEZ3, Oryctolagus cuniculus, SV=1, (0) A0A286Y431, Cavia porcellus, SV=1, (p) A0A340XS26, Lipotes vexillifer, SV=1, (q) A0A2U3Y890, Leptonychotes weddellii, SV=1, (r) A0A1A6FZ83, Neotoma lepida, SV=1, (s) A0A2Y9J3D1, Enhydra lutris kenyoni, SV=1, (t) A0A2U3VOR3, Tursiops truncatus, SV=1, (u) A0A383Z6R7, Balaenoptera acutorostrata scammoni, SV=1, (v) M3YVR5, Mustela putorius furo, SV=1, (w) Q29307, Sus scrofa, SV=2, (x) L8IJ24, Bos mutus, SV=1, (y) P01096, Bos taurus, SV=2, (z) A0A250Y8Y0, Castor canadensis, SV=1, (ai) G5AP86, Heterocephalus glaber, SV=1, (bi) 035143, Mus musculus, SV=2, (ci) G3H1Z3, Cricetulus griseus, SV=1, (di) S9XNE5, Camelus ferus, SV=1, (ei) A0A1S2ZPB9, Erinaceus europaeus, SV=1, (fi) A0A1U8CVF2, Mesocricetus auratus, SV=1, (gi) A0A091E4M7, Fukomys damarensis, SV=1, (hi) G1U0F8, Oryctolagus cuniculus, SV=1, (ii) G1PGS1, Myotis lucifugus, SV=1, (ji) F7BE70, Monodelphis domestica, SV=1, (ki) W5NYG6, Ovis aries, SV=1. In other embodiments, not shown, instead of a H49K substitution, there is a H49A or H49R substitution instead. (10F) SEQ ID NO:781. Compare and contrast this figure with FIG. 10B. Wherein the present figure shows features of some preferred IF1 protein variants, which have a “phosphorylation control switch” locked in the “on” position, and an attenuated “pH dependence motif”. (10G) SEQ ID NO:782 to SEQ ID NO:816. Especially preferred is an IF1 protein with its “phosphorylation control switch” residue set to alanine (A), which cannot be phosphorylated, and so cannot be switched “off”, and the starred (*) histidine (H) of its “pH dependence motif” (FIG. 10B) substituted with lysine (K). This figure shows illustrative IF1 proteins modified in this way. These sequences are componentry to the present disclosure as are other modified IF1 proteins, not shown, which are modified equivalently, as are nucleotide sequences that code for them by the genetic code, as are their protein/nucleotide sub-sequences e.g. with N-terminal Mitochondrial Import Sequence (MIS) absent. Any IF1 protein, e.g. any IF1 protein sequence from InterPro family “Mitochondrial ATPase inhibitor (IPR007648)” and/or Pfam family “IATP (PF04568)”, with its “phosphorylation control switch” residue (FIG. 10B) being alanine (A) and the starred (*) histidine (FIG. 10B) of its “pH dependence motif” substituted with lysine (K), is componentry to this disclosure. Illustrative IF1 proteins modified at these two positions are shown in this figure (wherein the original unmodified sequences are also componentry to this disclosure, later sequence versions (SV) are also contemplated): (a) Q9UII2, Homo sapiens, SV=1, (b) P01096, Bos taurus, SV=2, (c) O35143, Mus musculus, SV=2, (d) Q03344, Rattus norvegicus, SV=2, (e) G1SEZ3, Oryctolagus cuniculus, SV=1, (f) A0A286Y431, Cavia porcellus, SV=1, (g) E2QYN4, Canis lupus familiaris, SV=1, (h) M3WIS8, Felis catus, SV=2, (i) F6ZXT0, Equus caballus, SV=1, (j) G5AP86, Heterocephalus glaber, SV=1, (k) G3H1Z3, Cricetulus griseus, SV=1, (1) A0A1U8CVF2, Mesocricetus auratus, SV=1, (m) M3YVR5, Mustela putorius furo, SV=1, (n) A0A2Y9LD45, Delphinapterus leucas, SV=1, (o) G3SWQ8, Loxodonta africana, SV=1, (p) A0A2Y9EF27, Physeter catodon, SV=1, (q) Q5RFJ9, Pongo abelii, SV=1, (r) A0A2R9CQC9, Pan paniscus, SV=1, (s) A0A2J8Y5Q9, Pongo abelii, SV=1, (t) H2PYG9, Pan troglodytes, SV=1, (u) A0A213H1P7, Nomascus leucogenys, SV=1, (v) G3QEV8, Gorilla gorilla gorilla, SV=1, (w) A0A2K6AYV8, Macaca nemestrina, SV=1, (x) A0A2K5YI49, Mandrillus leucophaeus, SV=1, (y) A0A2K5P0W3, Cercocebus atys, SV=1, (z) G7NWV6, Macaca fascicularis, SV=1, (ai) A0A096NQ00, Papio anubis, SV=1, (bi) F6ZXX7, Macaca mulatta, SV=1, (ci) A0A0D9S814, Chlorocebus sabaeus, SV=1, (di) A0A2K6N3T3, Rhinopithecus bieti, SV=1, (ei) A0A2K6Q8H8, Rhinopithecus roxellana, SV=1, (fi) A0A2K5SG67, Cebus capucinus imitator, SV=1, (gi) A0A2K6SEK8, Saimiri boliviensis boliviensis, SV=1, (hi) A0A2K5KBI5, Colobus angolensis palliatus, SV=1, (ii) A0A2K5DQW7, Aotus nancymaae, SV=1. In other embodiments, not shown, instead of a H49K substitution, there is a H49A or H49R substitution instead. (10H) SEQ ID NO:817 to SEQ ID NO:836. Some preferred embodiments of this disclosure, applying teaching of FIG. 10F to the Human IF1 protein sequence. In further embodiments, shown, the N-terminal Mitochondrial Import Sequence (MIS) is absent. Not shown, but also contemplated, is wherein H56 (“mature” [without MIS] IF1 protein numbering), of the “pH dependence motif”, can optionally be substituted to alanine and/or wherein H49R or H49A is used instead of a H49K substitution. (10I) SEQ ID NO:837 to SEQ ID NO: 868. Bovine IF1 protein and some non-limiting fragment embodiments thereof. IF1 protein 1-60 fragment can't dimerize and exists as a monomer [143], 10-46 has ten-fold less activity than 10-47 showing the importance of the 47^{th} residue, 14-47 has been termed the “minimal inhibitory sequence” [142, 141], 22-46 can inhibit F1 ATP hydrolysis but not F₁F₀ ATP hydrolysis [144, 142]. 42-58 is an alternative “minimal inhibitory sequence” [144-147]. It might be that 42-58 inhibits F₁F₀ ATP hydrolysis by a different binding/mechanism than 14-47 and entire IF1 protein. (10J) SEQ ID NO:869 to SEQ ID NO:894. Human IF1 protein and some non-limiting fragment embodiments thereof. (10K) SEQ ID NO: 895 to SEQ ID NO:922. A sub-sequence/fragment of an IF1 protein, or sequence variant thereof, in either orientation (N to C, C to N), attached to a Mitochondrial Import Sequence (MIS), in either orientation, optionally the MIS of human IF1 protein (MAVTALAARTWLGVWGVRTMQARGF [SEQ ID NO: 162]) or that of a different IF1 protein, optionally that of a mammal, rodent or non-human primate, or attached to a MIS of another protein that is located to the mitochondrial matrix, is componentry to this disclosure (as are the nucleotide sequences that code for it), wherein illustrative example embodiments are shown. (10L) SEQ ID NO:923 to SEQ ID NO:950. An IF1 protein/fragment (and/or sequence variant thereof), in either orientation (N to C, C to N), attached to a (any) Cell Penetrating Peptide (CPP) sequence, in either orientation, optionally via a connecting glycine (increases flexibility between domains), wherein many such CPP sequences are known to those of the art: e.g. the HIV-1 Tat cell penetrating peptide sequence, YGRKKRRQRRR [SEQ ID NO:442], optionally flanked by glycines at one or both ends, at both ends: GYGRKKRRQRRRG [SEQ ID NO:445]. Furthermore, an IF1 protein fragment, or sequence variant thereof, in either orientation, attached to a Mitochondrial Import Sequence (MIS), in either orientation, optionally the MIS of human IF1 protein (or that of a different IF1 protein, optionally that of a mammal, rodent or non-human primate, or attached to a MIS of another protein that is located to the mitochondrial matrix), which is itself attached to a/any CPP sequence, in either orientation, optionally via a connecting glycine(s)/proline(s), is componentry to this disclosure (as are the nucleotide sequences that code for it), wherein illustrative example embodiments are shown. Encompassed by this disclosure: the domains can be ordered differently than shown in the figure e.g. the MIS can instead be “upstream” (closer to N terminus) of the CPP sequence, and all possible orientation (N to C, C to N) combinations are contemplated. (10M) SEQ ID NO:951 to SEQ ID NO: 978. An IF1 protein/fragment (or sequence variant thereof), in either orientation (N to C, C to N), attached to a/any CPP sequence, in either orientation, which is attached to an/any epitope/affinity tag sequence (many known to those of the art; non-limiting examples disclosed elsewhere herein, in the figure two examples are shown: HHHHHH [SEQ ID NO:131], HHHHHHDYKDDDDK [SEQ ID NO: 130]), in either orientation, optionally wherein the CPP sequence is flanked by 1-5 glycines and/or prolines (increases flexibility between domains). An IF1 protein/fragment (or sequence variant thereof), in either orientation (N to C, C to N), attached to a Mitochondrial Import Sequence (MIS), in either orientation, optionally the MIS of human IF1 protein (or that of a different IF1 protein, optionally that of a mammal, rodent or non-human primate, or attached to a MIS of another protein that is located to the mitochondrial matrix), which is itself attached to a/any CPP sequence, in either orientation, attached to an/any epitope/affinity tag sequence (many known to those of the art), in either orientation, optionally wherein the CPP sequence is flanked by 1-5 glycines and/or prolines. Encompassed by this disclosure: the domains can be ordered differently than shown e.g. the MIS can instead be “upstream” (closer to N terminus) of the CPP sequence and/or epitope/affinity tag sequence, the CPP sequence can be “upstream” of the epitope/affinity tag sequence etc. e.g. one or more domains can be attached to the C-terminus of IF1 protein/fragment (or sequence variant thereof). A benefit of having the MIS N-terminal to the IF1 protein/fragment (or sequence variant thereof), and the other domain(s) N-terminal to the MIS, is that when the MIS is cleaved off inside the mitochondrial matrix, the other domain(s) are cleaved off with it. Any IF1 protein/fragment (or sequence variant thereof), or concatenation(s) thereof, attached to a/any CPP and/or MIS and/or epitope/affinity tag sequence, optionally connected in one or more places by a connecting glycine(s)/proline(s) (or other connecting amino acid (aa) or short (<20 aa) amino acid sequence or linker sequence, optionally a protease/small molecule cleavage site), wherein all orientation (N to C or C to N) combinations are contemplated, is componentry to this disclosure, as are their coding nucleotide sequences. It will be appreciated by those skilled in the art that the order of these elements can be altered, and additional elements can be added so long as the functionality of the various elements is retained. How to produce these fusion proteins is known to those of the art, as for example described in [P14] (also see U.S. Pat. No. 6,498,020B1), the content and teachings of which are herein incorporated, in their entirety, by reference. (10N) SEQ ID NO:979 to SEQ ID NO:1071. Some embodiments. (10O) SEQ ID NO: 1072 to SEQ ID NO:1126. Some embodiments. (10P) SEQ ID NO:1127 to SEQ ID NO:1162. IF1 protein sequences from a number of long-lived species {are componentry to this disclosure, as is their use thereof [for at least one use disclosed herein]}: long-lived either in absolute terms and/or in relation to their size. Some of these sequences, such as that for the bowhead and blue whales, have never been reported before. Numbers in brackets are maximal lifespan in years (from [115]). Compared to the human number, which is drawn from a huge sample size, the other numbers, drawn from small sample sizes, are likely to be an underestimate of species maximal longevity. Some/all of the presented turtle/terrapin species may have “negligible senescence” [115]. The bolding highlights differences from the human IF1 protein sequence. Except for the very bottom, separated section, wherein bolding highlights differences from the presented naked mole rat IF1 protein sequence. Underlining is for a residue, different from human IF1 protein, and well conserved across whales/dolphins and long-lived reptiles/birds. By the teaching of this disclosure, a species with a longer lifespan tends to have a more potent IF1 protein, at normal mitochondrial matrix pH (8). This figure teaches some (not all) substitution(s)/addition(s) that can be made to human IF1 protein to confer a more potent IF1 protein, at normal mitochondrial matrix pH (8), which can confer slower aging and a longer human lifespan (if a higher ambient temperature and/or greater bodily insulation compensates for the upward shift in thermoneutral temperature). Or in a mouse, for example, with this modified human IF1 protein expressed. Changes in the first 25 residues of the presented human IF1 protein sequence are not desirable, because this is its Mitochondrial Import Sequence (MIS). In this figure, human is at the top, then going down: whales, dolphins, reptiles, bird, fish, “sea cow”, elephant, primates, rodents: (a) Homo sapiens. (b) Balaena mysticetus. (c) Balaenoptera physalus. (d) Balaenoptera musculus. (e) Megaptera novaeangliae. (f) Orcinus orca. (g) Physeter catodon. (h) Eschrichtius robustus. (i) Ziphius cavirostris. (j) Globicephala melas. (k) Balaenoptera acutorostrata scammoni. (l) Monodon monoceros. (m) Delphinapterus leucas. (n) Tursiops truncatus. (o) Lipotes vexillifer. (p) Chelonoidis abingdonii. The maximal lifespan shown is for a different species of Galápagos tortoise: Chelonoidis nigra. (q) Terrapene carolina triunguis. (r) Chelonia mydas. (s) Chrysemys picta bellii. (t) Trachemys scripta elegans. (u) Alligator mississippiensis. (v) Varanus komodoensis. (w) Crocodylus porosus. (x) Strigops habroptilus. (y) Anoplopoma fimbria. (z) Anguilla anguilla. (ai) Trichechus manatus latirostris. (bi) Loxodonta africana. (ci) Gorilla gorilla gorilla. (di) Cebus capucinus imitator. (ei) Saimiri boliviensis boliviensis. (fi) Callithrix jacchus. (gi) Carlito syrichta. (hi) Heterocephalus glaber. (ii) Cryptomys damarensis. (ji) Mus musculus. In other embodiments, not shown, the Mitochondrial Import Sequence (MIS) of these sequences is replaced by an MIS from a different species (preferably that for its native IF1 protein) e.g. a human/mouse MIS (e.g. that for its native IF1 protein). Preferably it is replaced by a MIS from the species that the protein sequence will be administered to/expressed in (e.g. that for its native IF1 protein). Also componentry to this disclosure is a fragment comprising (or consisting of) the 39^{th}-72^{nd} residues of the first sequence shown (human IF1, ≙14-47 with “mature” [without MIS] IF1 protein numbering, optionally wherein 1, 2, 3, 4 or 5 of its C-terminal residues are absent: ≙14-46, ≙14-45, ≙14-44, ≙14-43, ≙14-42), and the fragments that align with it in the sequences below, each as separate stand-alone peptide/protein sequences of this disclosure, wherein the concatenation of each at their N-terminal end with one or more of a MIS, CPP and affinity/epitope tag is also contemplated. If this fragment begins with a serine or threonine residue, in alternative embodiments, this is replaced with an alanine residue. Also componentry to this disclosure is the 67^{th}-83^{rd} residues of the first sequence shown (human IF1 protein, ≙42-58 “mature” IF1 protein numbering), and the fragments that align with it in the sequences below, each as separate stand-alone protein sequences of this disclosure, wherein the concatenation of each at their N-terminal end with one or more of a MIS, CPP and affinity/epitope tag is also contemplated. The IF1 protein sequence(s), and corresponding fragments thereof, of a different long-lived species (long-lived in absolute terms [preferred] and/or for its size), not shown here, is also componentry to this disclosure, as is its use thereof (for at least one use disclosed herein). In some embodiments, the protein administered/expressed is the same as the IF1 protein of the species being treated, or a fragment (or concatenation of fragments) thereof. (10Q) SEQ ID NO:1163 to SEQ ID NO:1198. The same sequences as the previous figure except that the 74^{th} residue of each is lysine (≙H49K) and, if a sequence's 39^{th} residue isn't already alanine, it is changed to be so (≙S14A, ≙T14A). In other embodiments, not shown, instead of a ≙H49K substitution, there is a ≙H49A or ≙H49R substitution instead. (10R) SEQ ID NO:1199 to SEQ ID NO: 1226. By the teaching of this application, some sequence variants of the bowhead whale IF1 protein, which are componentry to this disclosure, are shown. Note that this figure teaches more sequences than are actually shown because its accompanying text component, at its top (which includes mouse MIS sequence for its IF1 protein [SEQ ID NO:163]), teaches methods to arrive at further protein sequences of this disclosure that aren't shown (and this optional text instruction optionally applies to not just the sequences shown, but to other protein sequences in this application and/or of this disclosure). By this disclosure, one way that the inhibitory potency of an IF1 protein can be increased is by increasing the number of aspartic acid (D) residues at its C-terminus. Bowhead whale IF1 protein, and sequence variants thereof, are presented with one or more extra D residues at its C-terminus. In other protein sequence embodiments of this disclosure, one or more of the same modifications shown in this figure, at the equivalent position(s), is made for the IF1 protein of a different long-lived species e.g. a different long-lived species of whale (e.g. fin or blue whale). (10S) SEQ ID NO:1227 to SEQ ID NO:1263. At the very top is human IF1 protein and then bowhead whale IF1 protein immediately below it, with its residues distinct from human IF1 protein bolded, wherein its disparities from human IF1 protein that are well conserved across whales/dolphins and long-lived reptiles/birds are underlined. Thereafter are modified human IF1 protein sequences from the teaching of the bowhead whale sequence. This method of using an IF1 protein from a longer living species to instruct modification(s) to an IF1 protein from a shorter living species, to increase its inhibitory potency for F₁F₀ ATP hydrolysis at normal mitochondrial matrix pH (8), is componentry to this disclosure, as the use thereof of the resulting IF1 protein variant or fragment [or concatenated fragments] thereof (for at least one use disclosed herein). Some presented sequences have further modifications, beyond that by the teaching of the bowhead whale sequence, including one or more of ≙H49K, H55A, E26A substitutions, and/or one or more further D residues at the C-terminus, and/or D at the 79^{th} residue, which is what some long-living reptiles have at this position. Any combination/admixture of the bolded modifications to the human IF1 protein sequence in this figure, and not only the combinations shown, is componentry to this disclosure. When these protein sequences are administered/expressed in mice, it is preferable to substitute their human Mitochondrial Import Sequence (MIS) with such a sequence from mice, preferably that for their native IF1 protein. In other embodiments, not shown, instead of a ≙H49K substitution, there is a ≙H49A or ≙H49R substitution instead. (10T) SEQ ID NO:1264 to SEQ ID NO:1298. Some preferred (including the 14-47 and 42-58 [using “mature” {without MIS} IF1 protein numbering] sub-sequences) bowhead whale inspired sequences of the disclosure. Equivalent sequences derived using the IF1 protein sequence of a different long-lived species, e.g. a different long-lived whale (e.g. fin whale), are also componentry to this disclosure. (10U) SEQ ID NO: 1299 to SEQ ID NO:1327. Some preferred blue whale inspired sequences of the disclosure. In distinction to the bowhead whale, and like human, the blue whale has a residue at position 14 (“mature” IF1 protein numbering) that can be phosphorylated (threonine, human has serine). In some embodiments this is substituted for alanine (AT14A). The ≙42-58 sequence fragment, and associated derivatives, are not shown because these are the same as for the bowhead whale, presented earlier. (10V) SEQ ID NO: 1328 to SEQ ID NO:1362. Some preferred (including the 14-47 and 42-58 [using “mature” {without MIS} IF1 protein numbering] sub-sequences) human inspired sequences of the disclosure. Notably, for the 9^{th} sequence down, once the epitope tag sequence has been cleaved off (e.g. by Enterokinase), the sequence corresponds to an artificial concatenation of sequences that are naturally occurring in the human body. Thence, this is a very natural therapeutic. In some embodiments, it (and/or other peptide/protein sequence(s) of this disclosure, e.g. its 14-47 and/or 42-58 and/or 48-81 {or fragment thereof} fragment [“mature” (without MIS) IF1 protein numbering] with a concatenated human IF1 Mitochondrial Import Sequence (MIS) at its N-terminal end and then a CPP sequence concatenated to the N-terminal end of this, preferably a CPP correspondent to a sequence found in a protein naturally occurring in the human body) is administered topically/locally (thence bypassing proteases in systemic circulation), optionally to the skin/scalp, optionally as a cream, optionally as an anti-aging cosmetic/supplement (the resultant local reduction in metabolic heat production is mitigated by heat transfer from other body areas, especially via blood flow). In the sequences of this figure the CPP is R7, with a flanking glycine added to its C-terminal end (which is a “natural” sequence found within a human protein). In other embodiments, not shown, just R7 is used instead. Or R7 flanked at one or both of its ends with one or more glycine and/or proline residues (preferably less than 5, ideally 1 to 2). In other embodiments (not shown), Tat is used as the CPP instead, optionally flanked at one or both of its ends with one or more glycine and/or proline residues (preferably less than 5, ideally 1 to 2), optionally being YGRKKRRQRRRG [SEQ ID NO:446] or GYGRKKRRQRRRG [SEQ ID NO:445]. Indeed, in all figures herein, where the CPP is shown to be R7, in other embodiments it is a Tat sequence, or other CPP, and conversely, where the CPP is shown to be Tat, in other embodiments it is R7, or other CPP. In both cases, optionally flanked at one or both of its ends with one or more glycine and/or proline residues (preferably less than 5, ideally 1 to 2). In figures herein, where an affinity/epitope tag is shown, in other embodiments it is a different such tag of the art. (10W) SEQ ID NO:1363 to SEQ ID NO:1392. Some further preferred blue whale and human inspired sequences of the disclosure. (29X) SEQ ID NO:1393 to SEQ ID NO:1417. Illustrative, non-limiting, embodiments wherein a CPP sequence (R7 in this case, but in other embodiments a different CPP is used, for example, a longer poly-Arginine sequence is used, up to R50, optionally wherein one or more of these R residues have D-stereochemistry) is attached by a disulphide bond instead of a peptide bond. The bowhead whale derived variant sequences have the advantage that they use a cysteine internal to the IF1 sequence (not at the N- or C-terminal ends; this location to substitute in an internal cysteine was selected on the basis that IF1 of Gray whale has a cysteine in the equivalent position), wherein these cysteines are proximal to an aliphatic residue (flanked by alanines in one case, proximal to leucine in other), and so for both these reasons their disulphide bond is less susceptible to the problem of “disulfide bond exchange” (U.S. Pat. No. 9,255,124B2). In other embodiments, not shown, if there is not an aliphatic residue already next to a cysteine involved in a disulphide bond then one or more is inserted on one or both sides, optionally and independently in each case selected from alanine, valine, leucine, isoleucine. The attachment of more than one CPP sequence is contemplated. In the whale IF1 sequence variant with two cysteine residues, their connection directly to each other by a disulphide bond is contemplated (not shown), as is-alternatively-their parallel connection via a CPP linker sequence with cysteine residues in it, optionally at its either end (not shown), as is, in other embodiments, their further connection, or only one of them, via a CPP linker sequence that has an available cysteine, to a cysteine residue added to or near the N-terminus. (10Y) SEQ ID NO: 1418 to SEQ ID NO:1425. Some non-limiting peptide inhibitors of F₁F₀ ATP hydrolysis: melittin, pre-sequence of subunit IV of yeast cytochrome c oxidase, Syn-A2, Syn-C and Δ11,12 [4], bovine F₁ β subunit residues 394-413, 384-403, 404-423 [148]: these, and sequence variant(s) thereof, and concatenation(s) thereof, are componentry to this disclosure, as are nucleotide sequences that code for them. Encompassed by this disclosure: the domains can be ordered differently than shown e.g. the Mitochondrial Import Sequence (MIS) can instead be “upstream” (closer to N terminus) of the Cell Penetrating Peptide (CPP) Sequence and/or epitope/affinity tag sequence, the CPP sequence can be “upstream” of the “epitope/affinity tag sequence” etc. Any F₁F₀ ATP hydrolysis inhibitory peptide (or fragment thereof) or concatenation of such sequences (or concatenated fragments thereof), optionally sequence variant(s) thereof, attached to a CPP sequence and/or MIS and/or epitope/affinity tag sequence, wherein all orientation (N to C or C to N) combinations are contemplated, is componentry to this disclosure, as are their coding nucleotide sequences. (10Z) A few non-limiting example embodiments shown. In some embodiments one or more of the E in the EEE sub-sequence is (each independently) replaced with an amino acid whose side-chain is not negative, optionally leucine (L), glutamine (Q) or asparagine (N), optionally a non-proteingenic amino acid, optionally 5,6-dehydrohomoleucine (CAS: 73322-75-5; available from suppliers on www.labnetwork.com e.g. from Arena Chemical, La Mure, France) or(S)-2-amino-5-methylhexanoic acid (CAS: 31872-98-7; available on www.labnetwork.com e.g. from Astatech Inc., Bristol PA, USA). In some embodiments, in place of one or more histidines, (S)-2-amino-3-(1H-imidazol-1-yl) propanoic acid (CAS 114717-14-5; PubChem CID: 12311022; available from BOC sciences, Shirley, NY, USA, PubChem SID: 254789149) is incorporated into the peptide/protein chain instead. Optionally, one or more of the NH are replaced with NCH₃, especially preferred at one or more places on the peptide backbone i.e. one or more N° are methylated. Optionally, instead of N(CH₃)₂ as shown here, N(CH₃)₃ is at the N and/or C terminal ends. Or N(H)R, NR₂, CH₃, C(H₂)R, C(H)R₂, CR₃, R, wherein R is independently selected at each point of use from the options for R given in this disclosure in one of its paragraphs relating to modifying an N- and/or C-terminal end of a peptide/protein. D-amino acid in place of the corresponding L-amino acid, at one or more places, is componentry to this disclosure.

### EXAMPLE EMBODIMENTS OF THIS DISCLOSURE

The Drawings present some embodiments of this disclosure. Further examples are enumerations of Markush Formulas (I), (II), (III), (IV), (V) and (VI), presented henceforth. Note: none of these formulae share Markush symbols, which can be, for example, symbols of the type: Rₓ, wherein x is an integer and/or letter. Such symbols are well recognised by those of the art. Markush Formulas (I), (II), (III), (IV), (V) and (VI), presented henceforth, each have their own Markush symbols, as specified for each, in their own respective sections of this disclosure. Further examples are peptide/protein/polynucleotide embodiments of Formula (VII) and (VIII), including embodiments wherein one or more of a gene or nucleotide/DNA/RNA sequence is administered to the subject to administer a peptide/protein embodiment of Formula (VII) and/or (VIII) to the subject.

In this disclosure, the term “Formula [X]” is used when a statement is true for Formula (I), (II), (III), (IV), (V), (VI), (VII) and (VIII), and all are being referred to independently. A compound of Formula [X] is a compound of Formula (I), or Formula (II), or Formula (III), or Formula (IV), or Formula (V), or Formula (VI), or Formula (VII), or Formula (VIII), or any compound presented in this disclosure's Drawings, or any compound componentry to this disclosure.

This disclosure is described using these example embodiments but it isn't limited to these. These merely illustrate the disclosure. Compounds of other structures, which are identified as therapeutic/cosmetic inhibitors by the rationale and methods of the present disclosure, are also encompassed by the present disclosure.

An aspect of this disclosure is at least one compound of at least one of Formula (I), (II), (III), (IV), (V), (VI), (VII), (VIII), [X], and/or a pharmaceutically-acceptable salt, solvate, hydrate or prodrug thereof, and/or a pharmaceutical/cosmetic composition(s) comprising at least one compound of at least one of Formula (I), (II), (III), (IV), (V), (VI), (VII), (VIII), [X], and/or a F₁F₀ ATP hydrolysis inhibitor(s) (that preferably inhibits F₁F₀ ATP synthesis less or, more preferably, not at all), and/or a compound(s)/composition(s) that reduces F₁F₀ ATP hydrolysis, optionally any peptide/protein/polynucleotide comprising {or consisting of} at least one amino acid/nucleotide sequence in the Sequence Listing component of this application {or sequence variant thereof and/or fragment/concatenated fragments thereof} and/or a pharmaceutical/cosmetic composition thereof, for use in treating, ameliorating, preventing, reversing or combating a disease or disorder, or physiological process (and/or one or more of its consequences) or an unwanted/undesirable aesthetic(s), selected from the following list;

Encompassed by this disclosure is a method of treating, ameliorating, preventing, reversing or combating a disease or disorder, or physiological process (and/or one or more of its consequences) or an unwanted/undesirable aesthetic(s), in a subject, selected from:
(i) cancer, any cancer, neoplasia, metastasis, tumor formation/growth/implantation, tumorigenesis, solid tumor, blood borne tumor, cancer that is refractory or resistant to conventional chemotherapy, drug resistant tumor, multidrug resistant cancer;
(ii) cancer that metabolizes much of its glucose and/or glutamine to lactate, for example a cancer exhibiting the Warburg effect and/or a cancer that can be discriminated from surrounding tissue by PET imaging (e.g. ¹⁸F-FDG PET) and/or a cancer that uses more glucose than surrounding normal tissue and/or a glycolytic cancer and/or a non-oxidative cancer and/or a cancer that favours glycolytic rather than oxidative metabolism (wherein this correlates with cancer danger and poor prognosis and so dangerous cancers with poor prognosis are hereby contemplated) and/or a cancer releasing a lot of lactate (e.g. causing elevated blood lactate in the subject) and/or a cancer residing in extracellular acidity and/or a cancer with a low bioenergetic cellular index (BEC) value/score and/or a cancer using aerobic glycolysis and/or a cancer residing in hypoxia (e.g. hypoxic tumour, e.g. solid tumour) and/or a cancer that is predominantly/disproportionally utilizing ATP synthase in its “reverse” ATP consuming, instead of its “forward” ATP producing, mode and/or a cancer that's proliferation/viability/danger is reduced by administering a compound that selectively/preferentially inhibits/reduces F₁F₀ ATP hydrolysis, wherein the compound inhibits/reduces F₁F₀ ATP synthesis much less (e.g. >1000 fold less, e.g. >5000 fold less), minimally or not at all and/or a cancer that has a relatively (e.g. as compared to normal cells of the tissue from which the cancer derives) hyperpolarized membrane potential across its mitochondrial inner membrane and/or a cancer that has relatively (e.g. as compared to normal cells of the tissue from which the cancer derives) low intracellular Reactive Oxygen Species (ROS) concentration and/or a cancer with relatively (e.g. as compared to normal cells of the tissue from which the cancer derives) less susceptibility to a polyketide F₁F₀ ATP synthase inhibitor (e.g. Oligomycin A) because its metabolism is more glycolytic and less oxidative (e.g, wherein the cancer demonstrates the Warburg effect) and/or a cancer with high HIF-la gene expression (which incidentally tends to correlate with less susceptibility to a polyketide F₁F₀ ATP synthase inhibitor);
(iii) cancer originating in one of peripheral blood, bone marrow, lung, colon, Central Nervous System (CNS), brain, skin, ovary, kidney, prostate, breast/mammary gland; including metastatic forms of these cancers; cancer found in lymph node/bone/soft tissue/metastatic site(s) and/or found in/causing pleural effusion, ascites; Carcinoma, Adenocarcinoma, Squamous cell carcinoma, Large cell carcinoma, Cystadenocarcinoma, Clear cell carcinoma, Sarcoma, Blastoma, cancer of epithelial/fibroblast/promyeloblast/lymphoblast/T lymphoblast/B lymphocyte cell type, Multi Drug Resistant (MDR) cancer, Anaplastic cancer, Hematopoietic cancer, Acute Lymphoblastic Leukemia (ALL), Childhood/Adult T acute lymphoblastic leukemia, Precursor T-cell acute lymphoblastic leukemia, Acute Myeloid Leukemia (AML), Acute promyelocytic leukemia, Chronic Myeloid Leukemia/Chronic Myelogenous Leukemia (CML), CML in blast crisis, Cancer with Philadelphia chromosome (BCR-ABL1 positive), Myeloma, Multiple myeloma, Plasma cell myeloma, Plasmacytoma, Lymphoma, Large cell immunoblastic lymphoma, Anaplastic large cell lymphoma, ALK-positive anaplastic large cell lymphoma, ALK positive cancer, Non-Small Cell Lung Cancer (NSCLC), lung carcinoma, lung adenocarcinoma, Minimally invasive lung adenocarcinoma, Non-small cell lung carcinoma, lung squamous cell carcinoma, Mesothelioma, Pleural epithelioid mesothelioma, Pleural mesothelioma, Bronchioalveolar carcinoma, Large cell lung cancer, Large cell lung carcinoma, Colon carcinoma, Colorectal carcinoma, Colon adenocarcinoma, Colorectal adenocarcinoma, Dukes' type C colorectal adenocarcinoma, Dukes' type D colorectal adenocarcinoma, Astrocytoma, Glioblastoma, Gliosarcoma, Glioblastoma multiforme, Melanoma, Malignant melanoma, Cutaneous melanoma, Amelanotic melanoma, Ovarian adenocarcinoma, Ovarian endometrioid adenocarcinoma, Endometrioid carcinoma of ovary, High grade ovarian serous adenocarcinoma, Ovarian serous cystadenocarcinoma, Renal cell carcinoma, Renal cell adenocarcinoma, Papillary renal cell carcinoma, Clear cell renal cell carcinoma, Clear cell renal carcinoma, Multi Drug Resistant (MDR) renal cancer, Prostate carcinoma, Prostate adenocarcinoma, Androgen Receptor negative (AR−) prostate cancer, Breast carcinoma, Breast adenocarcinoma, Ductal carcinoma, Invasive ductal carcinoma, Luminal A breast cancer, Estrogen Receptor positive (ER+) breast cancer, Progesterone Receptor positive (PR+) breast cancer, Hormone Receptor positive (HR+) breast cancer, Hormone responsive breast cancer, Triple Negative Breast Cancer (TNBC), Hormone Receptor negative (HR−) breast cancer, Hormone resistant breast cancer, Estrogen Receptor negative (ER−) breast cancer, Progesterone Receptor negative (PR−) breast cancer, HER2 negative (HER2−) breast cancer;
(iv) cancer that causes/drives fever, for non-limiting example, Non Hodgkin Lymphoma (NHL), Hodgkin Lymphoma (HL), Multiple Myeloma (MM), acute or chronic leukaemia, Acute Myelogenous Leukemia (AML), hairy cell leukemia, blast crisis of Chronic Myelogenous Leukemia (CML), ovarian cancer, kidney cancer (renal cell cancer), liver cancer (hepatocellular carcinoma), cancer that has spread to the liver, soft tissue sarcoma, bone cancer, adrenal gland tumour (such as phaeochromocytoma), pancreatic carcinoma, bronchogenic carcinoma, atrial myxoma, brain tumour, glioblastoma multiforme, tumour in the hypothalamus (e.g. chordoid glioma), solid tumour, tumour causing an obstruction or blockage somewhere in the body, Castleman's disease;
(v) HIV associated cancer: AIDS-defining cancer (ADC, e.g. Kaposi Sarcoma (KS), Non-Hodgkin Lymphoma (NHL), aggressive B-cell non-Hodgkin lymphoma, primary lymphoma of brain, primary central nervous system lymphoma, Burkitt's lymphoma, Burkitt's-like lymphoma, diffuse large B-cell lymphoma (DLBCL), cervical cancer, cervical carcinoma, invasive cervical carcinoma), Non-AIDS Defining Cancer (NADC, type of cancer more likely to occur in people who are infected with HIV than in people who are not infected, e.g. Hodgkin lymphoma, HPV related cancer/neoplasm, oncogenic DNA virus associated/driven/generated cancer, cancer of the mouth, throat, liver, lung, head, neck, anus, rectum, colorectal cancer);
(vi) cancer that causes/drives cachexia;
(vii) cancer in a child/adolescent, childhood/pediatric cancer;
(viii) chemotherapy and/or radiotherapy and/or immunotherapy resistant/refractory cancer, chemoresistant and/or radioresistant cancer;
(ix) cancer occurring/associated with inflammation and/or with Tumour Associated Macrophages (TAMs);
(x) cachexia, cancer driven/associated cachexia, cachexia occurring with an end-stage illness (e.g. cancer, heart failure, chronic obstructive pulmonary disease (COPD), liver failure, kidney failure, stroke, rheumatoid arthritis, severe burn injury and HIV/AIDS), cancer fatigue, weight loss, weight loss for known or unknown reason, chronic wasting disease, atrophy, brown atrophy, frailty, frailty syndrome, aging frailty, geriatric syndrome, age-related cachexia and/or sarcopenia, weakness, wasting, Intensive Care Unit (ICU)-Acquired weakness, muscle wasting following severe trauma in ICU patients, post-intensive care syndrome (PICS), anorexia-cachexia, anorexia nervosa, bulimia nervosa, eating disorder, amenorrhea, underweight, low body mass index (BMI, e.g. <18.5), low body fat percentage, body composition change, wasting syndrome, HIV wasting syndrome, malnutrition, clinical malnutrition, starvation, kwashiorkor syndrome, marasmus syndrome, malabsorption, malabsorption due to parasitic/bacterial infection (e.g. helminthiasis, Whipple's disease, small intestine bacterial overgrowth (SIBO), giardiasis etc.), anemia, refeeding syndrome, appetite loss, catabolysis, muscle atrophy, asthenia, muscle weakness (myasthenia), weakness, sarcopenia, osteoporosis, cachexia associated with HIV, AIDS, multiple sclerosis, rheumatoid arthritis, familial amyloid polyneuropathy, chronic kidney disease, cystic fibrosis, multiple sclerosis, motor neuron disease, Parkinson's disease, dementia, Addison's disease, mercury poisoning (acrodynia), chronic pancreatitis, untreated/severe type 1 diabetes mellitus, hormonal deficiency, tuberculosis, gastroenteritis, diarrhea, dysentery, any digestive disease or disorder, any gastrointestinal disease or disorder including functional gastrointestinal disorders, coeliac disease, tropical sprue, irritable bowel syndrome, inflammatory bowel disease, Crohn's disease, ulcerative colitis, short bowl syndrome, congestive heart failure, constrictive pericarditis, bradycardia, chronic obstructive pulmonary disease (COPD), altitude sickness, hyperthyroidism (subclinical hyperthyroidism, Graves' disease, multinodular goiter, toxic adenoma, inflammation of the thyroid {thyroiditis}, pituitary adenoma), fatigue, chronic fatigue syndrome or any disease or disorder or pathology in which a body tissue(s) is undersupplied or underutilises (vs. its need) an energetic/chemical substrate(s), including O₂;
(xi) cancer associated fever, which is especially associated with, but not limited to, Non Hodgkin Lymphoma (NHL), Hodgkin Lymphoma (HL), Multiple Myeloma (MM), acute or chronic leukaemia, Acute Myelogenous Leukemia (AML), hairy cell leukemia, blast crisis of Chronic Myelogenous Leukemia (CML), ovarian cancer, kidney cancer (renal cell cancer), liver cancer (hepatocellular carcinoma), cancer that has spread to the liver, soft tissue sarcoma, bone cancer, adrenal gland tumour (such as phaeochromocytoma), pancreatic carcinoma, bronchogenic carcinoma, atrial myxoma, brain tumour, glioblastoma multiforme, tumour in the hypothalamus (e.g. chordoid glioma), solid tumour, tumour causing an obstruction or blockage somewhere in the body, Castleman's disease;
(xii) disease or disorder or physiological process or condition that drives to and/or that causes a higher than normal body temperature such as (to illustrate and not restrict) high environmental temperature (e.g. hot climate), ingesting an uncoupler (e.g. 2,4-dinitrophenol), infection, sepsis, neutropenic sepsis, stroke, fever, pyrexia, hyperpyrexia, hyperthermia, malignant hyperthermia, neuroleptic malignant syndrome, serotonin syndrome, toxic serotonin syndrome, thyroid storm, heatstroke, surgery related, menopause (“hot flushes”), infection (non-limiting e.g. roseola, measles, enteroviral infections, parasitic, viral, fungal, Chlamydial, Rickettsial, bacterial, mycobacterial, systemic bacterial, intravascular, HIV associated, nosocomial), pyrogenic infection, thermoregulatory disorder(s), connective tissue disease(s), Kawasaki syndrome, drug overdose, drug or drug withdrawal induced hyperthermia, alcohol/drug withdrawal, idiosyncratic drug reaction, fever of known or unknown or uncertain origin (non-limiting e.g. infectious disease(s), inflammation, immunological disease(s), non-infectious inflammatory disease(s) {non-limiting eg. systemic rheumatic and autoimmune diseases, vasculitis, granulomatous diseases, pyogenic granuloma(s), autoinflammatory syndromes}, tissue destruction, reaction to incompatible blood product(s), metabolic disorder(s), inherited metabolic disorder(s), cancer, neoplasm, endogenous or exogenous pyrogen(s), injury, head injury);
(xiii) normal or higher than normal body temperature and feeling hot, optionally uncomfortably hot (e.g. because of sweating more than desired/comfortable e.g. because the subject's body is overly exerted to stop/slow body temperature rising e.g. because the body's physiological cooling mechanisms are being heavily utilized), because of high environmental temperature (e.g. because of hot/tropical climate e.g. because of summer season);
(xiv) disease/disorder/injury/pathology/surgery treatable/ameliorated/prevented/combated/helped by conferring hypothermia in a subject for some medical or other purpose which can include (to illustrate and not restrict) extending survivability after injury/trauma (e.g. extending the time that a subject can safely await specialist medical facilities/attention/treatment after injury e.g. extending, beyond an hour, the “golden hour” for getting a seriously injured soldier [e.g. by gunshot(s) and/or injury/injuries due to explosion(s)] to a medical facility), slowing a chemical reaction(s) rate in a subject for therapeutic benefit, preventing/minimizing/slowing brain and/or tissue damage, slowing physiological/pathological processes (reaction rates are temperature dependent) and so “buying time” to get the subject to treatment for their emergency (e.g. trauma/traumatic blood loss/septic shock or other medical emergency), treating/reducing the severity of/preventing a secondary injury or secondary tissue/psychological damage resulting from a primary injury/disease/trauma/surgery (non-limiting e.g. where the secondary injury or tissue damage is one or more of systemic inflammatory response syndrome (SIRS), sepsis, bacterial/fungal/viral infection, post-intensive care syndrome {PICS}, depression, anxiety, post-traumatic stress disorder, intensive care unit (ICU)-acquired neuromuscular weakness), slowing the progress of sepsis until a sufficient concentration of a working antibiotic(s) can be built up in the subject (furthermore hypothermia, by slowing sepsis progression, buys time to observe which antibiotic(s) can work, yielding time to try alternative further antibiotic option(s) if required), used soon after or just before clinical/legal death to preserve the subject's organs/tissues until the subject can be frozen/cryogenically frozen or the pathology that caused clinical/legal death (e.g. wound) can be fixed and the subject resuscitated, administered to a subject when a first responder (e.g. ambulance crew, e.g. soldier) deems the subject dead or unlikely to survive the journey to a medical facility (e.g. hospital) wherein this administration helps to preserve the subject which is helpful if hospital staff subsequently assess that they can, or might be able to, save the subject, stabilizing surgical/trauma/Emergency Room (ER) patients, deep hypothermic circulatory arrest for surgery (DHCA, non-limiting applications of DHCA include repairs of the aortic arch, repairs to head and neck great vessels, repair of large cerebral aneurysms, repair of cerebral arteriovenous malformations, pulmonary thromboendarterectomy, resection of tumors that have invaded the vena cava, brain tumor resection {wherein the anti-cancer activity of a compound(s) of this disclosure juxtaposes well}), Emergency Preservation and Resuscitation (EPR), hypothermia for a surgical purpose, protective hypothermia during surgery and/or surgery complication, hypothermia to slow/reduce blood loss, hypothermia for neuro- and/or cardio- and/or organ/tissue and/or life protection in a subject that has trauma/brain trauma/polytrauma/surgery/stroke/ischemic stroke/hemorrhagic stroke/cardiac arrest/myocardial infarction/hypoxia/shock (including, without limitation, low volume, cardiogenic, obstructive, and distributive shock)/sepsis/septic shock/multiple organ dysfunction syndrome/systemic inflammatory response syndrome (SIRS)/organ failure/cytokine storm/anaphylactic shock/seizure/disseminated intravascular coagulation/blocked airway/croup/rhabdomyolysis/[head/facial/spinal/chest/abdominal/ballistic/knife injury/trauma], or some other medical emergency/condition/disorder/disease/injury/operation, hypothermia for cardiac and/or cardiovascular surgery and/or open heart surgery and/or brain surgery (neurosurgery) and/or surgery using total circulatory arrest and/or surgery using cardiopulmonary bypass, Emergency Preservation and Resuscitation (EPR), preserving detached body parts such as limbs and/or organs (for example during organ storage/transport and/or transplant, thus increasing the time window for transplantation of organs to recipients; compound(s) of this disclosure is administered to organ to be transplanted [by administration to donor and/or by administration to isolated organ] and/or to organ recipient, optionally during transplant operation), protective hypothermia, targeted temperature management, therapeutic hypothermia, therapeutic hypothermia as adjunct treatment (e.g. protective adjunct) to a medical proceedure/surgery (non-limiting examples: amputation, vascular neurosurgery, aortic aneurysm repair, cardiovascular surgery, cardiac surgery with cardio-pulmonary bypass, cardioplegia for bypass surgery, coronary artery bypass graft (CABG) surgery, angioplasty, post-angioplasty), hypothermia therapy for stroke, acute ischemic stroke, acute global ischemia and hypoxia, burn(s), radiation injury, traumatic brain injury (TBI), blunt force trauma, trauma, trauma caused by an external physical source, trauma caused by surgery/elective surgery/planned surgery/emergency surgery, battlefield wound(s), bullet/knife wound, bleeding, hemorrhage, blood loss, blood clotting disorder, hypovolemia, hypovolemic shock, hemorrhagic shock, hematologic shock, shock, multi-system organ failure, multiple organ dysfunction syndrome, subarachnoid haemorrhage, aneurysm, ruptured/leaking aneurysm, aneurysmal subarachnoid haemorrhage, intracranial pressure elevation, intracranial aneurysm repair, intracerebral hemorrhage, traumatic intracranial hypertension, spinal cord injury, cardiac arrest, heart attack, myocardial infarction, acute myocardial infarction, heart failure (optionally left-sided, right-sided, systolic, diastolic or congestive heart failure), acute coronary syndrome, unstable angina, cardiogenic shock, hepatic encephalopathy, acute liver failure encephalopathy, acute liver failure, reperfusion injury, reperfusion injury post acute myocardial infarction, hypoxic/ischemia/ischemic/reperfusion injury, inflamation, neonatal peripartum encephalopathy, neonatal encephalopathy due to intrapartum asphyxia, neonate with perinatal asphyxial encephalopathy, neonatal encephalopathy, neonatal hypoxia-ischemia, neonatal hypoxia/ischemia, birth asphyxia, hypoxic-ischemic encephalopathy (HIE), haemorrhage, hypovolemia, exsanguination, decompression sickness, acute respiratory distress syndrome (ARDS), burn injury(s) including skin burn, inflammation, allergic reaction, anaphylaxis, tissue/organ rejection, hypoxia, hypoxemia, anoxemia, anoxia, anemia, hypervolemia, altitude sickness, obstructed airway, asthma attack, hypoxia in a body/tissue/organ, hypoglycemia, reperfusion injury (ischemia-reperfusion injury), upon release of a ligature or tourniquet, uraemia, crush syndrome, compartment syndrome, traumatic brain and/or spinal cord injury, major trauma, infection, bacterial and/or viral infection(s) (non-limiting e.g. meningitis), sepsis, septic shock, systemic inflammatory response syndrome (SIRS), stroke, cerebrovascular disease, ischemic brain injury, ischemic stroke, cerebral/brain ischemia, traumatic injury, brain injury, spinal cord injury, cardiac arrest, heart failure, congestive heart failure, Dilated cardiomyopathy, valvular heart disease, pulmonary embolism, adrenal crisis, Addisonian crisis, hypertensive emergency, haemorrhagic (hypovolemic) shock, cardiogenic shock, neurogenic shock, hepatic encephalopathy, blood loss, ischemic brain/heart/kidney/intestinal injury, autoimmune disease, status epilepticus, encephalitis/meningitis, chronic obstructive pulmonary disease (COPD), uremia, kidney disease, liver disease, pancreatitis, gastritis, infection (bacterial, viral or fungal), post-intensive care syndrome (PICS), intensive care unit (ICU)-acquired neuromuscular weakness (optionally in form of/because of one or more of critical illness polyneuropathy (CIP), critical illness myopathy (CIM), prolonged neuromuscular blockade, prolonged mechanical ventilation, disuse atrophy, prolonged immobility, poor mobility, recurrent falls, quadriparesis, tetraparesis), neuroprotection and/or cardioprotection and/or tissue protection during/after a stroke and/or ischemia and/or cardiac arrest and/or resuscitation and/or a period(s) of poor blood flow anywhere in a subject, hypoxic/ischemic condition caused by disease, injury, or a medical procedure (e.g. surgery);
(xv) poisoning by a toxic amount of a compound(s) in a subject (non-limiting e.g. carbon monoxide/methanol/heavy metal/ethylene glycol/pesticide poisoning, snake/spider/bee/insect/lizard venom, metabolic poison(s), nerve agent, chemical weapon, bacterial toxin(s) (e.g. food poisoning, Salmonella poisoning), endotoxemia, eukaryote produced toxin(s) e.g. (non-limiting) brevetoxin, drug(s)/substance(s) overdose e.g. (non-limiting) heroin, ethanol, a prescription medication(s), an over the counter medication(s) such as aspirin, paracetamol etc.; hypothermia is protective to toxic insult);
(xvi) hypermetabolism (optionally because of one or more of, without restriction, traumatic brain injury, injury to the body, infection, sepsis, burn, multiple trauma, fever, long-bone fracture, hyperthyroidism, prolonged steroid therapy, surgery, bone marrow transplant, recovery from anorexia/bulimia), heat intolerence, insomnia, fatal insomnia, nervousness, Luft's disease, non-thyroidal hypermetabolism, thyrotoxicosis, hyperthyroidism, overactive thyroid, subclinical hyperthyroidism, too much thyroid hormone(s) in the subject,
too much triiodothyronine (T3) and/or thyroxine (T4) in the subject, hyperthyroxinemia (including, without restriction, familial dysalbuminemic hyperthyroxinemia, familial euthyroid hyperthyroxinemia, thyroid hormone resistance syndrome), thyroid storm, hyperthyroidism caused by one or more of (without restriction) Graves' disease, thyroiditis,
Hashimoto's thyroiditis, subacute thyroiditis, postpartum thyroiditis, lumps (nodules) on the thyroid, enlarged thyroid gland (goitre), simple goitre, multinodular goiter, toxic multinodular goiter, toxic adenoma, toxic thyroid adenoma, inflammation of the thyroid, hyperplasia of thyroid, metastatic thyroid cancer, thyroid tumour, thyroid cancer (including, without restriction, papillary carcinoma, follicular carcinoma, medullary thyroid carcinoma, anaplastic thyroid carcinoma), eating too much iodine, goitrogen ingestion, consumption of ground beef contaminated with thyroid tissue (“hamburger hyperthyroidism”), too much synthetic thyroid hormone in the subject, pituitary adenoma, drug induced, Amiodarone drug induced, struma ovarii, Jod-Basedow syndrome, nonautoimmune autosomal dominant hyperthyroidism;
(xvii) low or less than desired metabolic/bioenergetic efficiency in a subject, or low or less than desired physical or mental performance (e.g. memory, IQ), or low or less than desired body weight, or fatigue/tiredness/weakness/exhaustion; compound(s) administration to cause greater metabolic/bioenergetic efficiency in the subject, enhancing their physical and/or mental performance and/or causing body weight gain;
(xviii) accelerated/premature aging, any accelerated aging disease, any progeroid syndrome, including (to illustrate and not restrict) premature aging because of chemo-/radio-/cancer therapy, Werner syndrome, Bloom syndrome, De Barsy syndrome, Rothmund-Thomson syndrome, Cockayne syndrome, xeroderma pigmentosum, trichothiodystrophy, combined xeroderma pigmentosum-Cockayne syndrome, restrictive dermopathy, Wiedemann-Rautenstrauch syndrome, Hutchinson-Gilford progeria syndrome (progeria), a laminopathy, Ataxia telangiectasia-like disorder 2, XFE progeroid syndrome, Muscular dystrophy, Muscular Dystrophy (Becker's, Duchenne, Limb-Girdle), Yamamoto's Muscular Dystrophy, Mandibuloacral dysplasia, Dilated cardiomyopathy, GAPO syndrome, Cutis laxia, Ehlers-Danlos syndrome, Lenz-Majewski hyperostatic dwarfism, SHORT syndrome, Progressive external opthalmoplegia, Nester-Guillermo progeria syndrome, MDPL syndrome, Dyskeratosis congenital, Down syndrome;
(xix) disease or disorder of aging (incidence/severity increases with increased age/senescence) and/or unwanted/undesirable aspect(s) of aging {and/or a disease/disorder associated with elevated Reactive Oxygen Species [ROS]} including (to illustrate and not restrict) age-associated decline, age-related/correlated disease/disorder/condition, aging frailty, frailty, frailty syndrome, wasting, sarcopenia, muscle weakness, weakness, muscle fatigue, weight loss, cachexia, functional decline, osteoporosis, sclerosis, kyphosis, reduction in bone density, cognitive decline, neurological decline, cognitive deficit, cognitive impairment, mild cognitive impairment, depression, degenerative diseases, neurodegenerative diseases, motor-associated neurodegenerative diseases, motor neuron disease, motor neuron dysfunction, amyotrophic lateral sclerosis (ALS), primary lateral sclerosis, progressive muscular atrophy, age-related muscular atrophy, age-related fat loss, progressive bulbar palsy, progressive supranuclear palsy, pseudobulbar palsy, hereditary spastic paraplegia, Parkinson's disease, parkinsonism, Multiple System Atrophy (MSA), Progressive Supranuclear Palsy (PSP), essential tremor, resting tremor, Alzheimer's disease, Huntington's disease, spinocerebellar ataxias, Friedreich's ataxia, cerebellar ataxia, dysautonomia, dementia, frontotemporal dementia, chronic traumatic encephalopathy, memory loss, aged cognition, age/aging related cognitive decline/impairment, congential epilepsy, Batten disease, polyglutamine diseases, atherosclerosis, atherosclerotic plaque in a blood vessel, arteriosclerosis, vascular stiffening, arterial stiffness, stiffened arteries, hypertension, cardiovascular disease(s), myocardial infarction, acute myocardial infarction, angina, arrhythmia, cardiomyopathy, congestive heart failure, coronary artery disease, carotid artery disease, endocarditis, coronary thrombosis, myocardial infarction, ischemia reperfusion injury, anemia, hypertension, aortic aneurysm, cardiac diastolic dysfunction, irregularity in heart rhythm, decrease in cardiac stress tolerance, increase in the cross-sectional area of cardiomyocyte(s), hypercholesterolemia, hyperlipidemia, mitral valve prolapsed, peripheral vascular disease, cardiac stress resistance, brain aneurysm, inflammatory or autoimmune disease, cerebrovascular disease, stroke, heart failure, heart failure with preserved ejection fraction, fibrosis, idiopathic pulmonary fibrosis (IPF), pulmonary fibrosis, fibrotic disease, cardiac fibrosis, liver fibrosis, pancreatic fibrosis, oral submucosa fibrosis, cystic fibrosis, gum recession, gingival recession, oral mucositis, pulmonary disease, age-related loss of pulmonary function, chronic obstructive pulmonary disease, emphysema, bronchiectasis, coronary artery disease, hypercholesterolemia, liver disease, fatty liver disease, metabolic syndrome, lysosomal storage disease, amyloidosis, systemic sclerosis, kidney disease, chronic kidney disease, renal disease, renal failure, end-stage renal disease (ESRD), renal insufficiency, glomerulosclerosis, cirrhosis, hepatic cirrhosis, hepatic insufficiency, immunosenscence, clonal hematopoiesis, Chronic Obstructive Pulmonary Disease (COPD), emphysema, breathlessness, asthma, hypertension, hypercholesterolemia, age-related thymic atrophy, chronic inflammatory disease(s), joint pain, arthritis, osteoarthritis, osteoarthritis of knee(s), arthritis (Osteo and Rheumatoid), Rheumatoid Arthritis, Juvenile Rheumatoid Arthritis (JRA), arthrosis, herniated intervertebral disc, kyphosis, hernia, herniated intervertebral discs, degenerative disc disease, vertebral disc degeneration, tendinopathy, androgenetic alopecia, male-pattern baldness, hair loss, Idiopathic Pulmonary Fibrosis, systemic sclerosis, Psoriasis, age-related loss of cardiac/pulmonary/cognitive/vision function, decrease in cardiac stress tolerance, insulin sensitivity, poor glycemic control, diabetes, type 1 diabetes, type 2 diabetes, diabetic ulcer, diabetic retinopathy, diabetic neuropathy, diabetic nephropathy (diabetic kidney disease), diabetic ulcer, boutonneuse fever, obesity, metabolic disease/syndrome/dysfunction, inflammatory bowel disease, andropause, retinal degeneration, sarcopenia, cachexia, age-related cachexia and/or sarcopenia, macular degeneration, age-related macular degeneration (AMD, early/intermediate/late), age-related wet macular degeneration, neovascular/wet AMD, dry age-related macular degeneration, dry AMD, Geographic atrophy (GA), dry age-related macular degeneration with geographic atrophy, wet and dry AMD in the same eye(s), Stargardt's macular degeneration, Best vitelliform macular dystrophy, retinopathy, diabetic retinopathy, proliferative diabetic retinopathy, diabetic macular edema, age/aging-related eye disease, ophthalmological/ophthalmic disease/disorder/condition, ocular disease, vision loss, blindness, progressive vision impairment, myopia (short-sightedness), degenerative myopia, hyperopia (far-sightedness), accommodative dysfunction, glaucoma, progressive glaucoma, cataract formation, cataract(s), retinal degeneration, progressive retinal degeneration, presbyopia, vision loss, retinitis pigmentosa, leber hereditary optic neuropathy, Fuchs spot, Best's disease, Sorsby's fundus dystrophy, vaso-obliteration in eye(s), oxygen induced vaso-obliteration, neovascularization in eye(s), hearing loss (e.g. age-related), deafness, presbycusis, tinnitus, naive T cell shortage, movement disability, nonalcoholic fatty liver disease (NAFLD), nonalcoholic steatohepatitis (NASH), immunosenescence, immune senescence, poor immune response to a vaccine(s) (so countering this improves vaccine response=improves the protection conferred by a vaccine), respiratory/urinary tract infection (RTI/UTI) especially in older/aged/elderly subjects, loss of bladder control, lower urinary tract symptoms (LUTS), Benign Prostatic Hyperplasia (BPH), hyperplasia, polycystic kidney disease, cancer, age-related cellular hypertrophy, dermatological disease/disorder, eczema, psoriasis, hyperpigmentation, nevi, rashes, atopic dermatitis, urticaria, diseases/disorders related to photosensitivity/photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, a hallmark(s) of aging, genomic instability, telomere attrition, epigenetic alteration(s), loss of proteostasis, deregulated nutrient sensing, mitochondrial dysfunction, cellular senescence, stem cell exhaustion, altered intercellular communication, homeostatic imbalance, reduced fitness, reduced reproductive fitness, infertility, incontinence, sleep disturbances, imbalance, fear, depression, ulcers;
(xx) aging and/or one or more signs of aging, wherein one or more of these compounds slow/delay/reduce/treat/prevent/stop/reverse aging, and/or extend lifespan and/or healthspan, and/or treat or delay the onset of geriatric aging of the human/animal body, tissue(s), or organ(s), and/or treat or delay the onset of an age-associated phenotype in a cell(s)/organism(s), and/or prolong fertility e.g. female fertility, delay menopause;
(xxi) skin aging and/or damage (including sun damage and/or photoaging), one or more signs of skin/scalp aging/age-correlated damage: non-limiting e.g. lateral canthal lines (crow's feet), liver/age spot(s), skin wrinkle(s) [e.g. facial wrinkles], fine lines in the skin (e.g. around the eyes and/or mouth), expression lines, laxity (sagging), rhytids (wrinkles), erythema (redness), dyspigmentation (brown discoloration [s]), dark circles under the eyes, “bags” under the eyes, solar elastosis (yellowing), keratoses (abnormal growths), poor skin texture, hair greying/loss etc;
(xxii) any pathology/condition/disease/disorder characterized by too much/inappropriate/undesired signals/activity/electrical activity in the nervous system including (to illustrate and not restrict) insomnia, fatal insomnia, sleep onset latency, delayed sleep phase disorder, exploding head syndrome, parasomnia, sleep-maintenance insomnia, sleep disorder, too much/inappropriate/undesired signals/activity/electrical activity in the nervous system, hyperactivity, hypersensitivity, premature ejaculation, hyperreflexia, Autonomic dysreflexia (AD), Hyperventilation syndrome, brain hyperactivity, overly sensitive sensory system, pathological crying and/or laughing, Pseudobulbar affect (PBA, emotional lability), Alzheimer's agitation, photophobia, phonophobia, temperature-sensitive, pressure-sensitive, brain hyperexcitability, overstimulation, intrusive thought(s), Perseveration, sensory overload, disorganized thinking, fantasy prone personality, malapdative daydreaming, dissociation, hyperkinetic disorder, agitation, Psychomotor agitation, restlessness, difficulty controlling behaviour, disruptive behaviour disorder, Emotional and behavioral disorder, pervasive developmental disorder, Overactive disorder associated with mental retardation and stereotyped movements, attention-deficit disorder, Attention Deficit Hyperactivity Disorder (ADHD), adult attention-deficit hyperactivity disorder, severe behavioral problem(s) in children (e.g., to illustrate and not restrict, combativeness and/or explosive hyperexcitable behavior {out of proportion to immediate provocation [s]}, hyperactive children who show excessive motor activity with accompanying conduct disorders consisting of one or more of: impulsivity, difficulty sustaining attention, aggressivity, mood lability, poor frustration tolerance), Premenstrual dysphoric disorder (PMDD), premenstrual syndrome (PMS), impulsiveness, impulsivity, impulse control disorder, lack of self-control, hysteria, histrionic personality disorder, attention difficulty, inattention, poor attention control, anxiety, paranoid anxiety, Paranoid personality disorder, distress, dysphoria, Adjustment disorder, separation anxiety, anxiety disorder, depressive anxiety, agitated depression, treatment-resistant depression, Generalized anxiety disorder, social anxiety disorder, stranger anxiety, separation anxiety (e.g. in dogs left at home), separation anxiety disorder, Mixed anxiety-depressive disorder, depression (all forms, all severities), restlessness/apprehension/anxiety before surgery, hypochondria, panic disorder, panic attack, emotional outburst, emotional instability, Intermittent explosive disorder, unreasonable/unwarranted anger/aggression, hyper-aggression, hostility, rage, poor temper control, self-hatred, poor attentional control, worry, irritability, neuroses, somatization disorder, somatic symptom disorder, pain disorder, psychological pain, psychogenic pain, psychogenic facial pain, Atypical odontalgia (AO), burning mouth syndrome, throbbing, toothache/pulpitis/dental pain, chronic lower back pain, negative emotion, persistent/enduring negative emotion, body dysmorphic disorder, factitious disorder, illness anxiety disorder, unwarrented fight-or-flight response, stress, emotional stress, emotional dysregulation, distress, psychological stress, acute stress, chronic stress, acute stress reaction, combat stress reaction, traumatic grief, grief, grief after death of loved one, Prolonged grief disorder (PGD), heartbreak, guilt, shame, remorse, emotional pain, Algopsychalia, psychalgia, suffering, emotional trauma, psychological trauma, broken heart, Post Traumatic Stress Disorder (PTSD), Complex post-traumatic stress disorder (C-PTSD), hypervigilance, sympathetic hyperactivity, inability or impaired ability to relax, flashbacks, dysphoric hyperarousal, agoraphobia, insomnia, fatal insomnia, mood disorder, irrational/unwarrented fear/terror, phobia, social phobia, Cancerophobia, thunderstorm/firework phobia, hypersexuality, hypersexual disorder, depression, clinical depression, unipolar depression, bipolar disorder, Bipolar I, Bipolar II, Bipolar disorder not otherwise specified (Bipolar NOS), cyclothymia, cyclothymic disorder, mixed affective state, atypical depression, melancholic depression, postpartum depression, double depression, seasonal affective disorder, mania, manic episode, hypomania, increase in energy of psychomotor activity, delirium, excited delirium, major depressive disorder, minor depressive disorder, recurrent brief depression, Depressive Disorder Not Otherwise Specified (DD-NOS), major depressive episode, persistent depressive disorder (PDD), dysthymia, dysthymic disorder, absence of euthymia, manic thoughts, racing thoughts, thought disorder, disordered thinking, reduced ability to plan and execute tasks, paranoia, hallucination (including, without limitation, visual, auditory, olfactory, gustatory, tactile, proprioceptive, equilibrioceptive, nociceptive, thermoceptive, chronoceptive), Charles Bonnet syndrome, delusion, persecutory delusion, hearing voices, homicidal/criminal ideation/tendency/thoughts, suicidal ideation/tendancy/thoughts, self-injury, non-suicidal self-injury, violence, attacking others, negative mood swing, personality disorder, Borderline personality disorder, Narcissistic personality disorder, malignant narcissism, dissociative disorder, dissociative identity disorder (DID), Psychosis, acute psychosis, chronic psychosis, psychosis spectrum disorder, manifestations of psychotic disorders, behavioral complications of mental retardation, stimulant psychosis, psychotic depression, hallucinogen persisting perception disorder, Psychoactive substance-related disorder, amphetamine-induced psychosis, brief psychotic disorder, Brief reactive psychosis, Menstrual psychosis, postpartum psychosis, Psychotic disorder, Psychopathy, chronic hallucinatory psychosis, manifestation(s) of psychotic disorder, neurotic/reactive/endogenous/involutional/psychotic depression/depressive disorder (optionally accompanied by anxiety or agitation), depressive neurosis, delusional depression, psychotic aggression, psychiatric symptoms of dementia, AIDS delirium, Supersensitivity psychosis, Tardive psychosis, Tardive dysmentia, Depersonalization disorder, out-of-body experience, Sociopathy, Schizophrenia, Paranoid schizophrenia, disorganized-type schizophrenia, simple-type schizophrenia, pseudoneurotic schizophrenia, prodromal schizophrenia, schizoaffective disorder, bipolar type schizoaffective disorder, depressive type schizoaffective disorder, schizoaffective psychosis, Schizotypal personality disorder, schizophreniform disorder, Delusional parasitosis, formication, paresthesias, Acroparesthesia, tinnitus, delusional disorder, delusional jealousy, inappropriate behaviour, behavioural disorder, antisocial personality disorder, Oppositional defiant disorder (ODD), conduct disorder (CD), Disruptive mood dysregulation disorder (DMDD), sadistic personality disorder, poor inhibitory control, kleptomania, Pyromania, trichotillomania, dermatillomania, pathological/problem gambling, dyskinesia, tardive dyskinesia, paroxysmal dyskinesia, Paroxysmal kinesigenic dyskinesia, Paroxysmal nonkinesigenic dyskinesia, Paroxysmal exercise-induced dystonia, Hemiballismus, tic disorder, tremor, clonus, Tourette's syndrome, coprolalia, copropraxia, Echophenomena, Echopraxia, Echolalia, Palilalia, stuttering/stammering, stereotypy, punding, Self-stimulatory behaviour (stimming), Stereotypic movement disorder (SMD), synesthesia, obsession, Obsessive-compulsive disorder (OCD), obsessive-compulsive personality disorder, anankastic personality disorder, relationship obsessive-compulsive disorder (ROCD), Scrupulosity, Primarily obsessional obsessive compulsive disorder, sexual obsession, Akathisia (including, without limitation, chronic, acute, Pseudoakathisia, Tardive akathisia, withdrawal or “rebound” akathisia), Restless legs syndrome, motor restlessness, periodic limb movement disorder (PLMD), periodic limb movements in sleep (PLMS), Sydenham's chorea, chorea, dystonia, Hypnic jerk, twitching, spasms, Tetanus, tetanus muscle spasms, tetanized state, Myoclonus, myoclonic seizure, Action myoclonus, Palatal myoclonus, Middle ear myoclonus, Spinal myoclonus, Stimulus-sensitive myoclonus, Sleep myoclonus, Cortical reflex myoclonus, Essential myoclonus, myoclonic epilepsy, Juvenile myoclonic epilepsy, Progressive myoclonus epilepsy (PME, including, without limitation, Dentatorubral-pallidoluysian atrophy, Unverricht-Lundborg disease, MERRF syndrome, Lafora disease), Reticular reflex myoclonus, Myoclonic epilepsy, diaphragmatic flutter, automatism, status epilepticus, Epilepsia partialis continua, Complex partial status epilepticus, epilepsy, epileptic seizure, simple partial seizure, complex partial seizure, generalized epilepsy, generalized seizure (including, without limitation, tonic-clonic, tonic, clonic, myoclonic, absence (including typical absence and atypical absence), atonic seizure), focal epilepsy, focal seizure, focal/partial seizure (including, without limitation, Simple partial seizure and Complex partial seizure), focal aware seizure, focal impaired awareness seizure, generalised epilepsy, temporal lobe epilepsy (including, without restriction, mesial temporal lobe epilepsy {MTLE} and lateral temporal lobe epilepsy {LTLE}), Frontal lobe epilepsy, Rolandic epilepsy, Nocturnal epilepsy, Nocturnal frontal lobe epilepsy, Autosomal dominant nocturnal frontal lobe epilepsy (ADNFLE), Generalized epilepsy with febrile seizures plus (GEFS+), Panayiotopoulos syndrome, epileptic fit, reflex epilepsy, reflex seizure, absence seizure (including, without limitation, childhood absence epilepsy, epilepsy with myoclonic absences, juvenile absence epilepsy, juvenile myoclonic epilepsy, Jeavons syndrome {eyelid myoclonia with absences}, genetic generalised epilepsy with phantom absences), complex partial seizure, atonic seizure, generalized tonic-clonic seizure, tonic-clonic seizure, extrinsic stimulus epilepsy, intrinsic stimulus epilepsy, photosensitive epilepsy, musicogenic epilepsy, thinking epilepsy, eating epilepsy, seizure(s), Febrile seizure, nerve agent induced seizure, Dravet syndrome (sometimes modest hyperthermic stressors like physical exertion or a hot bath can provoke seizures in affected individuals), acute symptomatic seizure, seizure-related disorder, drug related seizure, paroxysmal depolarizing shift, Ohtahara syndrome, Epilepsy in females with mental retardation, Rasmussen's encephalitis, Epilepsy syndrome, benign rolandic epilepsy, childhood absence epilepsy, absence epilepsy, juvenile myoclonic epilepsy, epileptic encephalopathies, Lennox-Gastaut syndrome, West syndrome (Epileptic spasms), Doose syndrome (Myoclonic astatic epilepsy, MAE), Lennox-Gestaut syndrome, pseudo-Lennox Gastaut syndrome (atypical benign partial epilepsy), Benign familial neonatal epilepsy, Benign occipital epilepsy of childhood, familial neonatal convulsions, Febrile infection-related epilepsy syndrome, interictal dysphoric disorder, euphoria sclerotic, psychogenic non-epileptic seizure, non-epileptic seizure, gelastic seizure, convulsion(s), migraine, status migrainosus, tension headache, headache, Hypnic headache, hiccups, intractable hiccups, thumps in equines, Postural orthostatic tachycardia syndrome (POTS), Pheochromocytoma, agony, pain, chronic pain, acute pain, pain due to disease/injury, neuropathic pain, fibromyalgia, postherpetic neuralgia, phantom pain, referred pain, back pain, lower back pain, pelvic pain, cancer associated pain, chemotherapy associated pain, Diabetic neuropathy, small fiber peripheral neuropathy, Mononeuritis multiplex, Wartenberg's migratory sensory neuropathy, Breakthrough pain, idiopathic pain, polyneuropathy, Neuritis, Mononeuropathy, Polyradiculoneuropathy, Radial neuropathy, neuropathy, visceral pain, Burning feet syndrome, Tarsal tunnel syndrome, Carpal tunnel syndrome, Guyon's canal syndrome, Cubital Tunnel Syndrome, Repetitive strain injury, Sciatica, Neurofibromatosis, Ulnar nerve entrapment, Erythromelalgia, Paroxysmal extreme pain disorder, Proctalgia fugax, dysesthesia, scalp dysesthesia, dysesthetic burning, hyperesthesia, hyperalgesia, Opioid-induced hyperalgesia, hyperpathia, allodynia, pain response from stimuli which do not normally provoke pain, Complex regional pain syndrome (said to be most painful condition known to man), Radiculopathy, neuralgia (including, without restriction, intercostal neuralgia, trigeminal neuralgia, atypical trigeminal neuralgia, glossopharyngeal neuralgia, occipital neuralgia, postherpetic neuralgia), ciguatera poisoning, irritable bowel syndrome (IBS), interstitial cystitis (IC), temporomandibular joint disorder, acute intermittent porphyria, Porphyria, Acute porphyria (including, without limitation, acute intermittent porphyria {AIP}, variegate porphyria {VP}, aminolevulinic acid dehydratase deficiency porphyria {ALAD}, hereditary coproporphyria {HCP}, drug induced), Chronic porphyria (including, without limitation, X-linked dominant protoporphyria {XLDPP}, congenital erythropoietic porphyria {CEP}, porphyria cutanea tarda {PCT}, and erythropoietic protoporphyria {EPP}), cutaneous porphyria, Porphyria cutanea tarda, allergy, allergic reaction, anaphylaxis, anaphylactic shock, hives, asthma, allergic rhinitis, rhinitis, urticaria, contact dermatitis, cough, sore throat, esophageal reflux disease, heartburn, chest pain, Esophageal motility disorder, Nutcracker esophagus, diseases involving gastrointestinal motility, Peptic ulcer disease, esophageal spasm, angina, itchiness, Pruritus, severe pruritus, Prurigo, Pruritic skin condition, chronic itch, eczema, pruritus in eczema, neuropathic itch, neurogenic itch, itchiness due to atopic dermatitis and/or lichen simplex chronicus, peripheral sensitization, central sensitization, sensory perception of absent stimuli, too much sensory stimulation, sensory stimulation brings discomfort, Neuromyotonia, Peripheral nerve hyperexcitability, Morvan's syndrome, Benign fasciculation syndrome, Cramp fasciculation syndrome, hyperhidrosis, undifferentiated somatoform disorder, somatoform disorder, somatic symptom disorder, conversion disorder, functional neurological symptom disorder, severe nausea and/or vomiting, severe nausea/emesis, peripheral neuropathy, chemotherapy-induced peripheral neuropathy, chemotherapy-induced nausea and vomiting (CINV), radiation therapy-induced nausea and vomiting (RINV), postnarcotic nausea, hyperemesis gravidarum, morning sickness, Cyclic vomiting syndrome, retching/dry heaving, Urinary incontinence, enuresis, nocturnal enuresis, tail chasing, reduce urine spraying/marking behavior, benzodiazepine withdrawal syndrome, barbituate withdrawal syndrome, Neuroleptic discontinuation syndrome, drug withdrawal discomfort/pain/symptoms, drug use disorder, alcohol use disorder, opoid use disorder, amphetamine use disorder, cocaine use disorder, alcohol withdrawal syndrome/symptoms, delirium tremens, opoid withdrawal syndrome/symptoms, drug craving, drug addiction, drug dependence, polysubstance dependence, drug overdose, smoking, tobacco (nicotine) addition, tobacco (nicotine) withdrawal symptoms, alcoholism, addiction, opoid addiction, cocaine/crack addiction, addictive behaviour, addictive personality, behavioural addiction, internet/computer/computer game/social media/media addiction, exercise addiction, compulsive behaviour (e.g. {non-limiting} checking, counting, washing, repeating), anti-social behaviour, criminality, sexual compulsion, impulsive sexual behaviour, compulsive buying, gambling addiction, sex related addiction, sexual urge, hunger, eating desire/compulsion, eating disorder, polyphagia, overeating, binge eating disorder, compulsive overeating, insatiable/excessive appetite, bulimia nervosa, anorexia nervosa, substance abuse, substance-induced delirium, substance-induced psychosis, substance-induced mood disorder, drug overdose, vertigo, motion sickness, seasickness, mental/nervous breakdown, Autism spectrum disorder, neurological disorder, cognitive disorder, mental disorder, mental health disorder, mental health condition involving impaired or altered neural plasticity, mood disorder, mental disorder disclosed in Diagnostic and Statistical Manual of Mental Disorders, Fifth Edition (DSM-5) or a later edition, a mental/behavioural disorder disclosed by the International Classification of Diseases (ICD) in ICD-10 Chapter V: Mental and behavioural disorders (World Health Organisation, WHO);
(xxiii) disease or disorder or condition or pathology or unwanted/undesirable effect/action/behaviour treatable/ameliorated/prevented/combated, in totality or in part (e.g. along with surgery), by anaesthesia, pre-anaesthesia, post-anaesthesia, hypoesthesia, hypoactivity, sedation, coma, tranquilization, behavioural submission, muscle relaxation, hibernation, artificial hibernation, torpor, synthetic torpor, stasis, suspended animation (e.g. used during spaceflight because e.g. reduces ionizing radiation damage to subject); compound(s) administration to confer one or more of sedation, anaesthesia, hypoactivity, hibernation, torpor, suspended animation, life extension in a subject;
(xxiv) hyperproliferative/hyperplasia disorder, non-cancerous proliferative disorder, hyperproliferative autoimmune disorder, hyperplasia, epidermal hyperplasia, dysplasia (e.g. epithelial dysplasia), nodule(s), wart(s), papilloma(s), squamous cell papilloma, genital wart(s), condyloma(s), condyloma acuminatum, cyst(s), polyp(s) {including, without restriction, digestive, colorectal, endometrial, cervical, nasal, laryngeal, inflammatory fibroid polyp [s])}, inherited/hereditary (including, without restriction, Familial adenomatous polyposis, Peutz-Jeghers syndrome, Turcot syndrome, Juvenile polyposis syndrome, Cowden disease, Bannayan-Riley-Ruvalcaba syndrome {Bannayan-Zonana syndrome}, Gardner's syndrome) and non-inherited (non-restrictive e.g. Cronkhite-Canada syndrome) polyposis syndrome, benign tumour, adenoma, organ enlargement by hyperplasia, Cushing's disease (enlarged adrenal cortex by hyperplasia), congenital adrenal hyperplasia, hyperplasia of breast, atypical ductal hyperplasia, intraductal papillomatosis, fibroadenomas, fibrocystic changes, hemihyperplasia, focal epithelial hyperplasia, sebaceous hyperplasia, sebaceous adenoma, intimal hyperplasia, unwanted/undesirable smooth muscle cell proliferation, smooth muscle cell hyperplasia, intimal smooth muscle cell hyperplasia, neointimal hyperplasia, proliferative vascular disorders, stenosis, stenosis because of cellular proliferation, vaginal stenosis, stenosis in a blood vessel, vessel stenosis, aortic valve stenosis, lessoned patency of a blood vessel, stenosis in a blood vessel because of cellular proliferation, vascular occlusion, restenosis, restenosis in a blood vessel that has been implanted with a stent, in-stent restenosis, post-angioplasty restenosis, systemic sclerosis, cirrhosis of the liver, adult respiratory distress syndrome, idiopathic cardiomyopathy, lupus erythematosus, retinopathy (e.g. diabetic retinopathy and/or other retinopathy[y/ies]), cardiac hyperplasia, fibrosis, pulmonary fibrosis, idiopathic pulmonary fibrosis, fibromatosis, neurofibromatosis, renal interstitial fibrosis, Cowden syndrome, hamartoma(s), choristoma(s), hemangioma(s), lymphangioma(s), rhabdomyoma(s), lymphangiomatosis, cystic hygroma, trichilemmoma, sarcoidosis, neurosarcoidosis, aggressive fibromatosis, desmoid tumour(s), unwanted/undesirable skin cell proliferation, hyperproliferative skin disorder, psoriasis (including, without restriction, plaque, guttate, inverse, pustular, napkin, seborrheic-like, nail, scalp and erythrodermic psoriasis), psoriatic arthritis, dactylitis, seborrhoeic dermatitis, dandruff, eczema, atopic dermatitis, rosacea, reactive arthritis (Reiter's syndrome), pityriasis rubra pilaris, hyperproliferative variants of disorders of keratinization (e.g., without restriction, actinic keratosis, senile keratosis, keratosis pilaris, seborrheic keratosis), scleroderma, benign prostatic hyperplasia, prostate enlargement, endometrial hyperplasia, atypical endometrial hyperplasia, benign endometrial hyperplasia, adenomyosis, atypical polypoid adenomyoma, endometriosis, endometriosis of ovary (endometrioma), endometrial polyp(s), polycystic ovary syndrome, ovarian cyst(s), cervical polyp(s), uterine fibroid(s), uterine hyperplasia, proliferative smooth muscle disorders (such as intimal smooth muscle cell hyperplasia, which can lead to blockage in, for non-limiting example, the urethra, the bile duct, the airway, the bronchial airways of the lung and/or a blood vessel(s), particularly following biologically or mechanically mediated tissue injury), restenosis (to illustrate, without restriction, that associated with balloon angioplasty and/or insertion of a stent, incidentally wherein a “drug-eluting balloon”/“drug-eluting stent”/“drug-eluting medical device” with at least one compound of/in this disclosure is herein contemplated);
(xxv) Tumour Associated Macrophages (TAMs) or any macrophage associated disease or disorder such as, without limitation, Macrophage Activation Syndrome (MAS), HIV, AIDS, HIV-associated neurocognitive disorders (HAND), HIV-associated dementia complex (HAD), AIDS dementia, HIV-associated chronic inflammation, HIV associated peripheral neuropathy, HIV associated cancers, AIDS-defining cancers, non-AIDS defining cancers, HIV infection/transmission/drug resistance (use for HIV pre- and/or post-exposure prophylaxis [PEP] e.g. after needlestick injury and/or sex with an HIV infected person [s], e.g. to reduce the probability of mother to baby HIV transmission during pregnancy/birth/breast feeding), any disease in which the pathogen(s) hides from the immune system in macrophages including, without limitation, HIV (HIV virus can lay latent in macrophages during antiretroviral therapy [ART], wherein HIV virus becomes undetectable in blood, and then repopulate the virus in blood when ART is interrupted or discontinued; HIV virus recombines and mutates in macrophages, which is a drive to HIV drug resistance), Mycobacterium tuberculosis (causes tuberculosis), Leishmania parasite (causes Leishmaniasis), Chikungunya virus (causes Chikungunya), Legionella pneumophila (causes Legionnaires' disease), adenoviruses, T. whipplei (causes Whipple's Disease), Brucella spp. (causes brucellosis), Staphylococcus aureus, Ebola virus, Hepatitis B virus, Hepatitis C virus, influenza virus strains, dengue virus, bacteria and antibiotic resistant bacteria (so to stress, treatment of antibiotic resistant bacteria is taught), any disease or condition in which activated macrophages are unwanted or undesirable, any disease/disorder partially/completely driven by, or made worse, by activated macrophages (or similar activated cell type e.g. pancreatic islet macrophages/Langerhans cells/dendritic cells/monocytes/histiocytes/Hofbauer cells/Kupffer cells/phagocytes/microglia/epithelioid cells/osteoclasts/macrophage like cells/cells of the mononuclear phagocyte system, and/or any cell type(s) of the innate immune system and/or of the monocyte lineage, especially inducible nitric oxide synthase (iNOS) and/or iNOS2 expressing and/or NO producing cells {e.g. monocyte-derived inflammatory dendritic cells}, any disease in which the pathogen(s) hides from the immune system in monocytes including, without limitation, Human Cytomegalovirus (HCMV), disease/disorder caused by a pathogen(s) [non-limiting e.g. e.g. Plasmodium falciparum {which can cause cerebral malaria}, e.g. Streptococcus pneumonia {which can cause bacterial meningitis} that drives macrophages/microglia and/or another cell of the mononuclear phagocyte system to drive pathology (e.g. inflammation);
(xxvi) virus/pathogen neuroinvasion via macrophage(s), as used for non-limiting example by HIV, Heptatitis C virus, SARS coronavirus, coronavirus;
(xxvii) neurocognitive or neurodegenerative diseases/disorders, for non-limiting example those caused by a virus;
(xxviii) virus/pathogen transmission from mother to fetus/baby via macrophage(s) as used for non-limiting example by zika (via Hofbauer cells) and HIV (macrophages in breast milk);
(xxix) acute or chronic or systemic inflammation or any inflammatory disease/disorder/syndrome or any autoinflammatory disease/disorder/syndrome or any autoimmune disease/disorder/syndrome;
(xxx) acute inflammation, chronic inflammation, systemic inflammation, inflammation because of infection or foreign bodies or injury or chemical or toxin or drug or stress or frostbite or burn or ionising radiation or surgery, inflammatory diseases/disorders/syndromes, Macrophage Activation Syndrome (MAS), autoinflammatory diseases/disorders/syndromes, age-related chronic inflammatory diseases (“inflammaging”), autoimmune diseases/disorders/syndromes, diseases/disorders of the innate immune system, sore throat, sore throat associated with cold or flu or fever, high-intensity exercise associated inflammation, inflammatory response to a virus/coronavirus infection (non-limiting e.g. SARS-COV-2), ulcerative colitis, inflammatory bowel disease (IBD), irritable bowel syndrome (IBS), rheumatoid arthritis, osteoarthritis, inflammatory osteoarthritis, psoriatic arthritis, atopic dermatitis, allergic airway inflammation, asthma, inflammation associated depression, neuroinflammation, neuropathic pain, exercise-induced acute inflammation, atherosclerosis, allergy, hay fever, anaphylaxis, inflammatory myopathies, drug-induced inflammation, systemic inflammatory response syndrome, sepsis-related multiple organ dysfunction/multiple organ failure, microbial infection, acute brain/lung/hepatic/renal injuries, lung inflammation, acute lung injury (ARDS), acne vulgaris, celiac disease, celiac sprue, chronic prostatitis, colitis, autoimmune hemolytic anemia, diverticulitis, glomerulonephritis, proliferative glomerulonephritis, membranous nephropathy, minimal change nephrotic syndrome, hidradenitis suppurativa, hypersensitivities, interstitial cystitis, Mast Cell Activation Syndrome, mastocytosis, otitis, pelvic inflammatory disease (PID), endometritis, reperfusion injury, rheumatic fever, rhinitis, sarcoidosis, transplant rejection, parasitosis, eosinophilia, type III hypersensitivity, ischaemia, chronic peptic ulcer, tuberculosis, Crohn's disease, hepatitis, chronic active hepatitis, immune hepatitis, alcoholic hepatitis, chronic viral hepatitis, ankylosing spondylitis, diverticulitis, fibromyalgia, systemic lupus erythematous (SLE), Alzheimer's disease, Parkinson's disease, neurodegenerative disease, cardiovascular disease, chronic obstructive pulmonary disease, bronchitis, acute bronchitis, bronchiectasis, bronchopneumonia, obliterative bronchiolitis, appendicitis, acute appendicitis, bursitis, cystitis, dermatitis, encephalitis, HIV encephalitis, gingivitis, meningitis, infective meningitis, myelitis, nephritis, neuritis, periodontitis, chronic periodontitis, phlebitis, prostatitis, RSD/CRPS, rhinitis, sinusitis, chronic sinusitis, tendonitis, testiculitis, tonsillitis, urethritis, vasculitis, respiratory bronchiolitis-associated interstitial lung disease and desquamative interstitial pneumonia, pneumonia, interstitial lung disease, Löfgren syndrome, Heerfordt syndrome, monocytosis, liver fibrosis, steatohepatitis, nonalcoholic steatohepatitis, silicosis, histiocytoses, Langerhans' cell histiocytosis, haemophagocytic lymphohistiocytosis, pulmonary langerhans cell histiocytosis, obesity, type II diabetes, type I diabetes, gout, pseudogout, Chron's disease, organ transplant rejection, epidermal hyperplasia, chronic fatigue syndrome, graft versus host disease (GvHD), transplant rejection, lymphadenopathy, rheumatoid arthritis (RA), osteoarthritis (OA), inflammatory osteoarthritis, lupus, multiple sclerosis (MS), myocarditis, uveitis, CNS disease(s), inflammation aspect to a CNS disease(s), hypothalamic inflammation, dementia, glaucoma, progressive glaucoma, amyloid related/driven disease, lipid storage disease(s), fibrosis, nonalcoholic fatty liver disease (NAFLD), nonalcoholic steatohepatitis (NASH), cirrhosis, cirrhosis of the liver, alcoholic cirrhosis, nephropath(y/ies), lupus nephritis, immune nephritis, fibrotic disorder(s), cardiovascular disease, heart disease, atherosclerosis, vulnerable plaque, plaque formation, lipid containing macrophage related disease(s)/disorder(s)/malad(y/ies), macrophage foam cells, diabetes, type 1 diabetes, type 2 diabetes, insulin resistance, macrophage aspect to insulin resistance, obesity, obesity associated inflammation, macrophage accumulation/large numbers of macrophages in adipose tissue (e.g. associated with obesity), granuloma(s), granulomatous diseases, sarcoidosis (including, without limitation, Annular sarcoidosis, Erythrodermic sarcoidosis, Ichthyosiform sarcoidosis, Hypopigmented sarcoidosis, Löfgren syndrome, Lupus pernio, Morpheaform sarcoidosis, Mucosal sarcoidosis, Neurosarcoidosis, Papular sarcoid, Scar sarcoid, Subcutaneous sarcoidosis, Systemic sarcoidosis, Ulcerative sarcoidos), neurosarcoidosis, pulmonary sarcoidosis, interstitial lung disease, pulmonary fibrosis, pulmonary tuberculosis, immune reconstitution syndrome of HIV, Jarisch-Herxheimer reaction, sepsis, Paget's disease of bone, osteolysis, monocytosis, histiocytosis, X-type histiocytoses, non-X histiocytoses, Langerhans cell histiocytosis, non-Langerhans-cell histiocytosis, malignant histiocytosis, malignant histiocytic disorders, histiocytomas, histiocytic lymphoma, hemophagocytic syndrome, hemophagocytic lymphohistiocytosis, lymphohistiocytosis, diffuse histiocytic sarcoma, Rosai-Dorfman disease, gliosis, Bergmann gliosis, Chronic Obstructive Pulmonary Disease (COPD), chronic inflammatory lung disease, recurrent fever syndrome (which can be hereditary or acquired, optionally characterized by recurrent fever associated with rash, serositis, lymphadenopathy and musculoskeletal involvement), familial mediterranean fever (FMF), TNF receptor-associated periodic syndrome (TRAPS), Hyperimmunoglobulinemia D with recurrent fever syndrome (HIDS), cryopyrin associated periodic syndrome (CAPS), Blau syndrome, Majeed syndrome, deficiency of interleukin-1 receptor antagonist (DIRA), mevalonate kinase deficiency, pyogenic-arthritis-pyoderma gangrenosum and acne syndrome (PAPA), periodic fever aphthous stomatitis pharyngitis adenitis (PFAPA) syndrome, Behcet's disease, Still's disease, Crohn's disease, Schnitzler's syndrome, Sweet's syndrome, NLRP12-associated autoinflammatory disorders, deficiency of interleukin-1 receptor antagonist (DIRA), pyoderma gangrenosum, cystic acne, aseptic arthritis, periodic Fever Associated with mevalonate kinase deficiency (hyperimmunoglobulin D Syndrome), Pyogenic Arthritis Pyoderma Gangrenosum Acne (PAPA) syndrome, Periodic Fever Aphthous Stomatitis, Pharyngitis and Adenopathy (PFAPA) syndrome, Adult-Onset Still's Disease (AOSD), Systemic Juvenile Idiopathic Arthritis (sJIA), Chronic Recurrent Multifocal Osteomyelitis (CRMO), Synovitis Acne Pustulosis Hyperostosis Osteitis (SAPHO) syndrome, Cryopyrin associated Periodic Syndrome (CAPS), Familial cold auto inflammatory syndrome (FCAS), Muckle-Wells syndrome (MWS), Familial cold urticarial, Neonatal onset multisystemic inflammatory disorder (NOMID), hereditary Periodic Fever Syndromes, Periodic Fever Syndromes, systemic autoinflammatory diseases, Addison's disease, Agammaglobulinemia, Alopecia areata, Amyloidosis, Ankylosing spondylitis, Anti-GBM/Anti-TBM nephritis, Antiphospholipid syndrome, autoimmune angioedema, autoimmune dysautonomia, autoimmune encephalomyelitis, autoimmune hepatitis, autoimmune hemolytic anemia, autoimmune inner ear disease (AIED), autoimmune myocarditis, autoimmune pancreatitis, autoimmune retinopathy, autoimmune urticaria, axonal & neuronal neuropathy (AMAN), Baló disease, Behcet's disease, benign mucosal pemphigoid, Bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss, Cicatricial pemphigoid, Cogan's syndrome, cold agglutinin disease, congenital heart block, coxsackie myocarditis, CREST syndrome, Berger's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, proliferative glomerulonephritis, membranous nephropathy, minimal change nephrotic syndrome, Goodpasture's syndrome, granulomatosis with polyangiitis, Graves' disease, Guillain-Barre syndrome, Hashimoto's thyroiditis, hemolytic anemia, immune hemolytic anemia, Henoch-Schonlein purpura (HSP), Herpes gestationis or pemphigoid gestationis (PG), hypogammalglobulinemia, IgA Nephropathy, IgG4-related sclerosing disease, Immune thrombocytopeniaurpura (ITP), Inclusion body myositis (IBM), Interstitial cystitis (IC), juvenile arthritis, juvenile diabetes (Type 1 diabetes), juvenile myositis (JM), Kawasaki disease, Multisystem Inflammatory Syndrome in Children (MIS-C), Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multiple sclerosis, Myasthenia gravis, Myositis, Narcolepsy, Neuromyelitis optica, Neutropenia, Ocular cicatricial pemphigoid, Optic neuritis, Palindromic rheumatism (PR) PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, peripheral neuropathy, perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive arthritis, Reflex sympathetic dystrophy, Relapsing polychondritis, Restless legs syndrome (RLS), Retroperitoneal fibrosis, Rheumatic fever, Rheumatoid arthritis, rheumatic arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Wegener's granulomatosis (or Granulomatosis with Polyangiitis (GPA)), idiopathic thrombocytopenia purpura, splenomegaly, systemic lupus erythematosus, lupus erythematosus, dermatomyositis, Sjorgren syndrome;
(xxxi) Systemic inflammatory response syndrome, cytokine release syndrome, cytokine storm, immune reaction to a drug(s) or therapy(s), immune reaction to an immune activating drug(s) or agent(s) or treatment(s) or intervention(s), immune reaction to immunotherapy and/or immune-oncology and/or immunomodulatory drug(s) and/or treatment(s), adverse reaction to adoptive T-cell therapy(s), adverse reaction to a chimeric antigen receptor T-cell therapy(s) (CAR-T cell therapy(s)), adverse reaction to a immune checkpoint inhibitor(s), adverse reaction to monoclonal antibody drug(s), tumor lysis syndrome;
(xxxii) cancer and Graft Versus Host Disease (GVHD) in transplantation therapy in a cancer patient;
(xxxiii) cardiovascular diseases and conditions associated with thrombosis and/or the formation of atherosclerotic plaques and/or ischemia and/or ischemic conditions and/or associated conditions including, without limitation, ischemia-reperfusion injury, myocardial ischemia, ischemic heart disease, chronic stable angina pectoris, first or recurrent myocardial infarction (MI), congestive heart failure, an acute coronary syndrome, muscle cell damage, necrosis, cardiac arrhythmia(s), non-Q wave MI, unstable angina, high blood pressure, coronary artery disease, coronary arterial thrombosis, ischemic hypoxia, cyanosis, gangrene, acute limb ischemia, stroke, ischemic stroke, cerebral/brain ischemia, vascular dementia, ischemic sudden death, transient ischemic attack (TIA), thrombophlebitis, ischemic colitis, mesenteric ischemia, angina pectoris, ischemic heart disease, ischemic neuropathy, hypoxic-ischemic encephalopathy, cerebral hypoxia, brain hypoxia, ischemia resulting from vascular occlusion, cerebral infarction, stroke and related cerebral vascular diseases (including cerebrovascular accident and transient ischemic attack), muscle cell damage, necrosis, ventricular hypertrophy, ventricular enlargement (including dilated cardiac myopathy and heart failure), Prinzmetal's angina, peripheral occlusive arterial disease (e.g., peripheral arterial disease, intermittent claudication, critical leg ischemia, prevention of amputation, prevention of cardiovascular morbidity such as MI, stroke or death), pericardial effusion, constrictive pericarditis, thrombosis, thrombotic or thromboembolic conditions, circulatory disease caused by blood clot (i.e. diseases involving fibrin formation, platelet activation, and/or platelet aggregation), thrombotic or thromboembolic symptoms of thromboembolic stroke (including that resulting from atrial fibrillation or ventricular mural thrombus), arterial cardiovascular thromboembolic disorders, venous cardiovascular thromboembolic disorders, thromboembolic disorders in the chambers of the heart, venous thrombosis (including deep vein thrombosis), arterial thrombosis, cerebral thrombosis, cerebral arterial thrombosis, pulmonary embolism, cerebral embolism, kidney embolism, arterial embolism, thrombophilia, disseminated intravascular coagulation, restenosis, atrial fibrillation, atherosclerotic vascular disease, atherosclerotic plaque formation, atherosclerosis, atherosclerotic plaque rupture, peripheral arterial disease, coagulation syndromes, intermittent claudication, transplant atherosclerosis, vascular remodeling atherosclerosis, diabetic complications comprising retinopathy, nephropathy and neuropathy, thromboembolic consequenses of surgery, interventional cardiology or immobility, thromboembolic consequenses of medication (such as oral contraceptives, hormone replacement and heparin), thrombotic consequenses of atherosclerotic vascular disease and atherosclerotic plaque rupture leading to tissue ischemia, prevention of atherosclerotic plaque formation, transplant atherosclerosis, thrombotic or thromboembolic complications of surgery including interventional cardiology, thromboembolic complications of pregancy including fetal loss, thromboembolic consequences of thrombophilia (e.g., Factor V Leiden, and homocystinenimia), prothrombotic consequences and/or complications of cancer, prevention of thrombosis on artificial surfaces (such as stents, blood oxygenators, shunts, vascular access ports, vascular grafts, artificial valves, etc.), coagulopathies (e.g., disseminated intravascular coagulation), coagulation syndromes, vascular remodeling atherosclerosis, restenosis and systemic infection, Kasabach-Merritt syndrome, occlusion (e.g. after a bypass) and reocclusion (e.g., during or after percutaneous transluminal coronary angioplasty), thromboembolic disorders resulting from conditions including but not limited to atherosclerosis, surgery or surgical complications, prolonged immobilization, arterial fibrillation, congenital thrombophilia, cancer, diabetes, effects of medications or hormones, complications of pregnancy and thrombosis resulting from prosthetic valves or other implants, indwelling catheters, stents, cardiopulmonary bypass, hemodialysis, or other procedures in which blood is exposed to an artificial surface that promotes thrombosis, acute coronary syndrome selected from myocardial infarction, congestive heart failure, and cardiac arrhythmia; or
(xxxiv) diseases or disorders or conditions treatable/ameliorated/prevented/combated by conferring/maintaining blood vessel patency in a subject, which can be useful during interventional cardiology or vascular surgery including bypass grafting, arterial reconstruction, atherectomy, vascular graft and stent patency, organ, tissue and cell implantation and transplantation, preservation of host and/or graft tissue as related to organ transplantation;
wherein the method comprises administering to the subject an effective amount (e.g. a therapeutically effective amount) of at least one compound of at least one of Formula (I), (II), (III), (IV), (V), (VI), (VII), (VIII), [X], and/or a pharmaceutically-acceptable salt, solvate, hydrate or prodrug thereof, and/or a pharmaceutical/cosmetic composition(s) comprising at least one compound of at least one of Formula (I), (II), (III), (IV), (V), (VI), (VII), (VIII), [X], and/or a F₁F₀ ATP hydrolysis inhibitor(s) (that preferably inhibits F₁F₀ ATP synthesis less or, more preferably, not at all), and/or a compound(s)/composition(s) that reduces F₁F₀ ATP hydrolysis, optionally any peptide/protein/polynucleotide comprising {or consisting of} at least one amino acid/nucleotide sequence in the Sequence Listing component of this application {or sequence variant thereof and/or fragment/concatenated fragments thereof} and/or a pharmaceutical/cosmetic composition thereof; another aspect is a method of treating, ameliorating, preventing or combating any such disease or disorder by administering to a subject in need thereof a therapeutically effective amount of a compound(s) and/or composition(s) as described herein;
this disclosure further includes the use of one or more of a compound/composition disclosed herein for the manufacture of a medicament, optionally for treating one or more of the diseases/disorders/conditions listed in the list immediately above/aforementioned.

An aspect of this disclosure is a pharmaceutical composition comprising at least one compound, as described herein, (optionally a therapeutically/cosmetically effective amount) and one or more of a pharmaceutically-acceptable carrier, excipient, diluent.

An aspect of this disclosure is a compound(s) and/or composition(s) as described herein for use in a method of treatment of the human or animal body by therapy.

An aspect of this disclosure is a compound(s) and/or composition(s) as described herein administered to a subject topically or systemically or both.

Another aspect is the use of a compound(s) and/or composition(s) as described herein for the manufacture of a medicament for the treatment, amelioration, prevention or combating of any disease or disorder, optionally a disease or disorder mentioned or inferred herein.

This disclosure encompasses at least one compound of/in this disclosure, e.g. at least one compound of at least one of Formula (I), (II), (III), (IV), (V), (VI), (VII), (VIII), [X], and/or a pharmaceutically-acceptable salt, solvate, hydrate or prodrug thereof, and/or at least one pharmaceutical composition thereof, in co-therapy/co-administration (optionally conferring synergy) with one or more treatments and/or one or more compounds/compositions approved for human use by the United States Food and Drug Administration (FDA) and/or European Medicines Agency (EMA), optionally in the same pharmaceutical composition, or sold/distributed together, optionally in the same packaging, optionally wherein the FDA and/or EMA approved treatment(s) and/or compound(s) is for treating one or more of cancer, peripheral neuropathy, HIV, AIDS, viral infection, bacterial infection, optionally wherein zero or more of the FDA and/or EMA approved drugs co-administered are a cytochrome P450 enzyme (e.g. CYP2C9) substrate/inhibitor.

A compound(s) that inhibits F₁F₀ ATP hydrolysis, for example a compound(s) of Formula (I-V, VII-VIII), administered or self-administered to a subject, for use in preventing/ending their pregnancy/conception, optionally co-administered (optionally in a pharmaceutical composition) with another compound(s) or combination of compounds with this use, many of which are known to those of the art e.g. progestin, antiprogestin, estrogen etc. Taken after unprotected sex for example, with a later window of effectiveness than present emergency contraceptives. Preferably this use is restricted to the time during which ES cells exist in embryogenesis, which is early.

### Example (I)

### Summary of Formula (I)

This disclosure embodiment relates to compounds having the following formula: Formula (I)

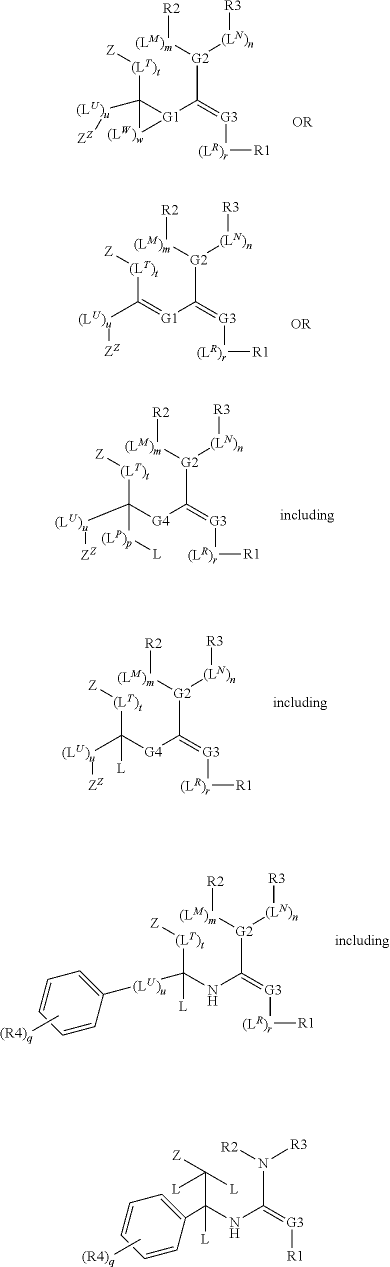

or a pharmaceutically-acceptable salt, solvate, hydrate, or prodrug thereof, wherein: G1 is N or CH;
G4 is NH or CH₂;
G2 is N or CH;
G3 is sulphur(S) or oxygen (O) or selenium (Se) or CH₂ and R1 is absent and r is 0; or
G3 is nitrogen (N), or CH or phosphorus (P) and R1 is present;
example embodiments include

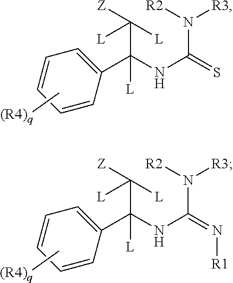

L^{M}, L^{N}, L^{U}, L^{T}, L^{W}, L^{P} and L^{R} are each independently selected from a single bond, O, S, Se, NR^{V}, PR^{V}, BR^{V}, C(R^{V})₂ or Si(R^{V})₂, wherein each RY is independently selected from hydrogen, deuterium, halogen (e.g. F), alkyl, or substituted alkyl (non-limiting examples: CF₃, CCl₃), or deuterated alkyl (non-limiting example: CD₃), or aminoalkyl, or thioalkyl, or alkoxy, or O, or OH (hydroxyl), or halogen, or haloalkyl, or haloalkoxy;
m, n, u, t, w, p and r are each independently selected from 0, 1, 2, 3 and 4;
L is independently at each point of its use alkyl, or substituted alkyl (non-limiting examples: CF₃, CCl₃), or deuterated alkyl (non-limiting example: CD₃), or aminoalkyl, or thioalkyl, or alkoxy, or halogen, or haloalkyl, or haloalkoxy, or hydroxyalkyl or any atom or isotope permitted by valence (including any accompanying hydrogen(s)/deuterium(s) by valence e.g. (non-limiting) OH, NH₂, SH, SiH₃, PH₂, BH₂ etc.) including, without limitation, La, Ti, Ce, V, Ta, Cr, Mo, Mn, Fe, Ru, Os, Co, Pd, Pt, Cu, Ag, Au, Zn, B, Al, Ga, C, Si, N, P, As, Sb, Bi, O, S, Se, F, Cl, Br, I, Hg;
R₁ is absent, Rₑₓₜᵣₐ, R₃, hydrogen, deuterium, cyano, aryl, heteroaryl, —SO₂R₈, —C(═O)R₉, —C(═CH₂)R₉, —C(—OH)R₉, —C(—SH)R₉, —C(—SeH)R₉, —C(—OL)R₉ (wherein L defined earlier), —C(═S)R₉, —C(═Se)R₉, —C(═NH)R₉, C(═PH)R₉, —S(═O)R₉, —C(═N—OH)R₉, —C(—N═O)R₉, —C(—P═O)R₉, —C(═N—O—CH₃)R₉, -(L^{J})ⱼR₉, —C(═R^{D})-(L^{J})ⱼ-R₉, -(L^{J})ⱼ-C(═R^{D})—R₉, or

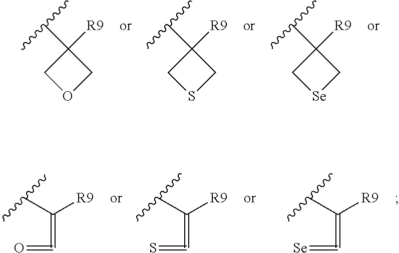

R^{D} is O, S, Se, NH or PH;
L^{J} is selected from a single bond, O, S, NR^{J} or C(R^{J})₂, wherein each R^{J} is independently selected from hydrogen, deuterium, halogen (e.g. F), alkyl, or substituted alkyl (non-limiting examples: CF3, CCl₃), or deuterated alkyl (non-limiting example: CD3), or aminoalkyl, or thioalkyl, or alkoxy, or halogen, or haloalkyl, or haloalkoxy;
j is 0, 1, 2 or 3;
Rₑₓₜᵣₐ is selected from L (defined earlier), aryl, heteroaryl, cycloalkyl, heterocyclo, arylalkyl, (heterocyclo)alkyl, (heteroaryl)alkyl, alkyl, substituted alkyl, alkenyl, substituted alkenyl, alkylene, substituted alkylene, alkynyl, substituted alkynyl, alkoxy, thioalkyl, aminoalkyl, carbamyl, sulfonyl, sulfonamide, cycloalkyl, (cycloalkyl)alkyl, hydroxyalkyl, haloalkyl, haloalkoxy, alkoxyalkyl, morpholinylalkyl, acyl, alkoxycarbonyl, substituted amino;
R₂ is (i) independently hydrogen, L (defined earlier), alkyl, or substituted alkyl,
   or (ii) taken together with R3 forms a heterocyclo;
R₃ is (i) independently R₁, alkyl, substituted alkyl, L (defined earlier), alkylthio, aminoalkyl, carbamyl, B^{B}-aryl, B^{B}-heterocyclo, B^{B}-heteroaryl, or B^{B}-cycloalkyl, or (ii) taken together with R₂ forms a heterocyclo;
Z is heteroaryl;
Z^{Z} is aryl, heteroaryl, cycloalkyl or heterocyclo;
B^{B} is a bond, C₁₋₄alkylene, C₂₋₄alkenylene, substituted C₁₋₄alkylene, substituted C₂₋₄alkenylene, —C(═O)NR₁₉—, —C₁₋₄alkylene-C(═O)N_{R19}—, or substituted C₁₋₄alkylene-C(═O)NR₁₉—;
R₄ at each occurrence is selected independently of each other R₄ from the group consisting of PH₂, OH, SH, halogen, alkyl, substituted alkyl, haloalkyl, nitro, cyano, haloalkoxy, OR₂₅, SR₂₅, NR₂₅R₂₆, NR₂₅SO₂R₂₇, SO₂R₂₇, SO₂NR₂₅R₂₆, CO₂R₂₆, C(═O)R₂₆, C(═)NR₂₅R₂₆, OC(═O)R₂₅, —OC(═O)NR₂₅R₂₆, NR₂₅C(═O)R₂₆, NR₂₅CO₂R₂₆, aryl, heteroaryl, heterocyclo and cycloalkyl;
R₈ is alkyl, substituted alkyl, aryl, or heteroaryl;
R₉ is —NR₁₀R₁₁, alkyl, substituted alkyl, alkoxy, alkylthio, cycloalkyl, aryl, heteroaryl, heterocyclo or —CO₂R₁₂;
R₁₀ and R₁₁, are (i) independently selected from hydrogen, alkyl, substituted alkyl, alkoxy, heterocyclo, cycloalkyl, aryl, and heteroaryl; or (ii) taken together form a heterocyclo or heteroaryl;
R₁₂ and R₁₉ are hydrogen or alkyl;
R₂₅ and R₂₆ are independently selected from hydrogen, alkyl, or substituted alkyl, or taken together form a heterocyclo or heteroaryl ring;
R₂₇ is alkyl or substituted alkyl, and
q is 0, 1, 2, or 3.

### Preferred Compounds of Formula (I)

Preferred methods are to use, and preferred compounds are, compounds with the following formula, or pharmaceutically-acceptable salts, solvates, hydrates or prodrugs thereof,

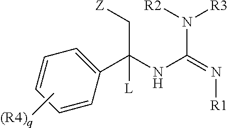

further preferred methods are to use, and preferred compounds are, compounds with the following formula, or pharmaceutically-acceptable salts, solvates, hydrates or prodrugs thereof,

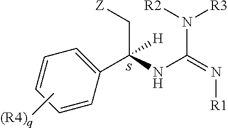

and even more preferred methods are to use, and preferred compounds are, compounds with the following formula, or pharmaceutically-acceptable salts, solvates, hydrates or prodrugs thereof,

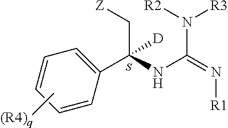

other preferred methods are to use, and preferred compounds are, compounds with the following formula, or pharmaceutically-acceptable salts, solvates, hydrates or prodrugs thereof,

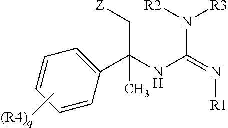

further preferred methods are to use, and preferred compounds are, compounds with the following formula, or pharmaceutically-acceptable salts, solvates, hydrates or prodrugs thereof,

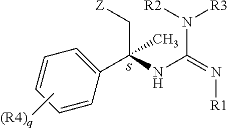

other preferred methods are to use, and preferred compounds are, compounds with the following formula, or pharmaceutically-acceptable salts, solvates, hydrates or prodrugs thereof,

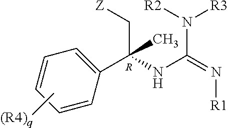

other preferred methods are to use, and preferred compounds are, compounds with the following formula, or pharmaceutically-acceptable salts, solvates, hydrates or prodrugs thereof,

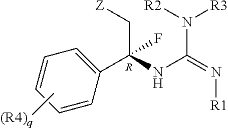

in which, in the preceding 7 structures shown:
L is hydrogen, or methyl, or alkyl, or hydroxyalkyl, or CF₃, or CD₃, or deuterium (D);
D is deuterium (enrichment, for example, exceeding 40% deuterium incorporation at shown position, and optionally at other positions also);
S symbolises the S stereoisomer, for example, in enantiomeric excess (ee) exceeding 70%;
R symbolises the R stereoisomer, for example, in enantiomeric excess (ee) exceeding 70% (following IUPAC naming rules the chiral carbon of the fluorine (F) analogue is labelled R rather than S, but note that the arrangement of which bond is up, bold wedge, and down, dashed, around the stereogenic carbon is the same as the preceeding structures labelled S at their chiral carbon, it is this molecule arrangement that is salient, and that is disclosed, rather than a mere label in a naming convention. This clarification won't be repeated at every place to which it applies in this disclosure, at every point at which there is an F in place of an H on the chiral carbon, or any other higher order of priority atom (by IUPAC rules), e.g. (non-limiting) any other halogen, because this clarification here itself is likely superfluous: all this is very clear to someone of the art. So, when there is said to be an enantiomeric excess (ee) in this disclosure in relation to this example embodiment, Formula (I), it applies to this molecular configuration, this arrangement of solid/dashed wedges, about the chiral carbon, whether this be S or R by IUPAC naming rules);
Z is triazolyl optionally substituted independently with one to two R₇ or imidazolyl optionally substituted independently with one to two R₇ and/or having fused thereto a benzene ring in turn optionally substituted independently with one to two R7;
R₁ is cyano or —C(═O)R₉;
R₂ is hydrogen, alkyl, or benzyl;
R₃ is aryl or arylalkyl optionally substituted independently with one or more groups selected from alkyl, halogen, trifluoromethyl, OCF₃, cyano, nitro, amino, hydroxy, methoxy;
R₄ is halogen, alkyl, trifluoromethyl, or OCF₃;
R₇ is alkyl, carbamyl or carbamylC₁₋₄alkyl;
R₉ is —NR₁₀R₁₁, alkyl, substituted alkyl, alkoxy, alkylthio, cycloalkyl, aryl, heteroaryl, heterocyclo or —CO₂R₁₂;
R₁₀ and R₁₁ are (i) independently selected from hydrogen, alkyl, substituted alkyl, alkoxy, heterocyclo, cycloalkyl, aryl, and heteroaryl; or (ii) taken together form a heterocyclo or heteroaryl;
R12 is hydrogen or alkyl; and
q is 0, 1, 2, or 3.

More preferred methods are to use, and preferred compounds are, compounds with the following formula, or pharmaceutically-acceptable salts, solvates, hydrates or prodrugs thereof,

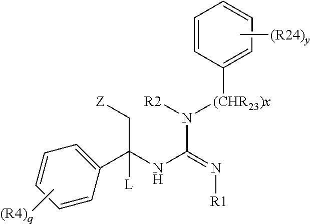

other preferred compounds are those with the following formula, or pharmaceutically-acceptable salts, solvates, hydrates or prodrugs thereof,

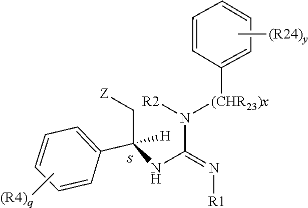

and even more preferred are compounds having the following formula, or pharmaceutically-acceptable salts, solvates, hydrates or prodrugs thereof,

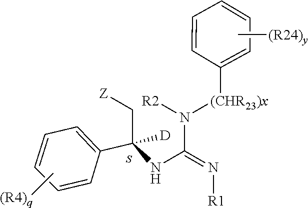

other preferred compounds are those with the following formula, or pharmaceutically-acceptable salts, solvates, hydrates or prodrugs thereof,

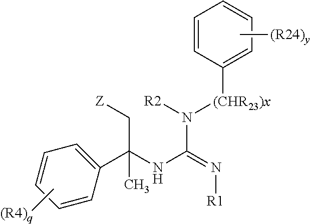

other preferred compounds are those with the following formula, or pharmaceutically-acceptable salts, solvates, hydrates or prodrugs thereof,

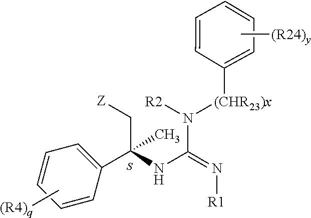

other preferred compounds are those with the following formula, or pharmaceutically-acceptable salts, solvates, hydrates or prodrugs thereof,

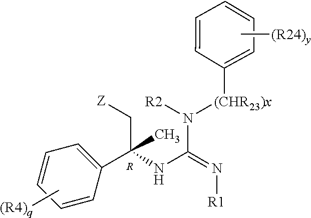

other preferred compounds are those with the following formula, or pharmaceutically-acceptable salts, solvates, hydrates or prodrugs thereof,

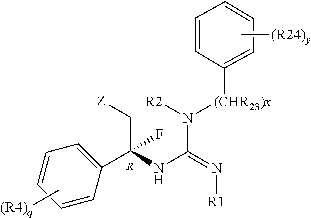

in which, for the preceding 7 structures shown:

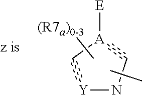

and more preferably

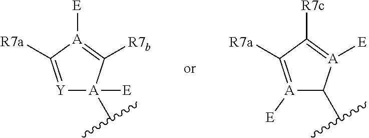

z is

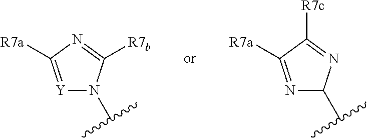

or

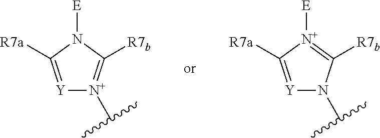

or
L is hydrogen, or methyl, hydroxyalkyl, or CF₃, or CD₃, or deuterium (D);
D is deuterium (enrichment, for example, exceeding 40% deuterium incorporation at shown position, and optionally at other positions also);
S symbolises the S stereoisomer, for example, in enantiomeric excess (ee) exceeding 70%;
A is nitrogen (N), or N⁺, or carbon;
E is absent, or alkyl, or substituted alkyl, or deuterated alkyl, or aminoalkyl, or thioalkyl, or alkoxy or any atom or isotope permitted by valence (including any accompanying hydrogens by valence e.g. (non-limiting) OH, NH₂, SH, SiH₃, PH₂ etc.), for example hydrogen, deuterium or fluorine;
Y is N, CH or CR_{7c};
R₁ is cyano or —C(═O)R₉;
R₂ is hydrogen or C₁₋₄alkyl;
R₄ is halogen, C₁₋₄alkyl, trifluoromethyl, or OCF₃;
R₇ₐ, R_{7b}, and R_{7c} are independently E (defined earlier), hydrogen, alkyl, carbamyl or carbamylC₁₋₄alkyl, or R₇ₐ and R_{7c} join to form an optionally substituted fused phenyl ring;
R₉ is —NR₁₀R₁₁, alkyl, substituted alkyl, alkoxy, alkylthio, cycloalkyl, aryl, heteroaryl, heterocyclo or —CO₂R₁₂;
R₁₀ and R₁₁ are (i) independently selected from hydrogen, alkyl, substituted alkyl, alkoxy, heterocyclo, cycloalkyl, aryl, and heteroaryl; or (ii) taken together form a heterocyclo or heteroaryl;
R₁₂ is hydrogen or alkyl;
R₂₃ is hydrogen, alkyl, hydroxyalkyl, or phenyl;
R₂₄ is (selected independently at each point of its use)alkyl, substituted alkyl, haloalkyl, halogen, trifluoromethyl, cyano, hydroxy, OCF₃, methoxy, phenyloxy, benzyloxy, cyano, or acyl, or two R₂₄ groups join to form a fused cycloalkyl or benzene ring;
q is 1 or 2;
x is 0, 1, or 2; and
y is 0, 1, 2, or 3.

More preferred methods are to use, and preferred compounds are, compounds with the following formula, or pharmaceutically-acceptable salts, solvates, hydrates or prodrugs thereof,

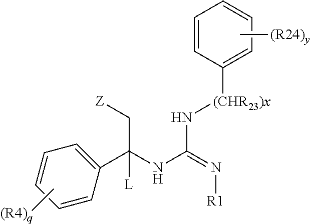

other preferred compounds are those with the following formula, or pharmaceutically-acceptable salts, solvates, hydrates or prodrugs thereof,

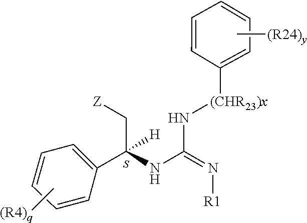

and even more preferred are compounds having the following formula, or pharmaceutically-acceptable salts, solvates, hydrates or prodrugs thereof,

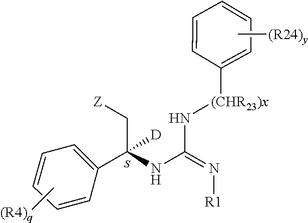

other preferred compounds are those with the following formula, or pharmaceutically-acceptable salts, solvates, hydrates or prodrugs thereof,

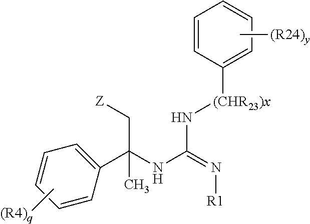

other preferred compounds are those with the following formula, or pharmaceutically-acceptable salts, solvates, hydrates or prodrugs thereof,

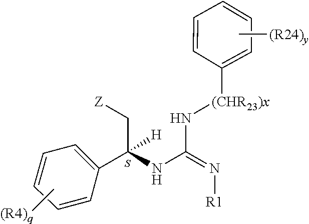

other preferred compounds are those with the following formula, or pharmaceutically-acceptable salts, solvates, hydrates or prodrugs thereof,

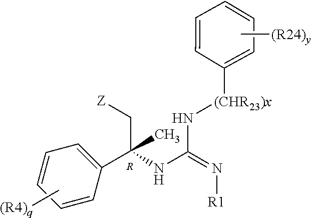

other preferred compounds are those with the following formula, or pharmaceutically-acceptable salts, solvates, hydrates or prodrugs thereof,

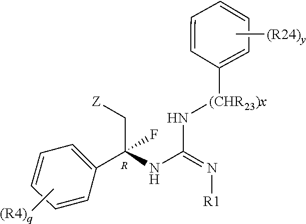

in which, for the preceding 7 structures shown:

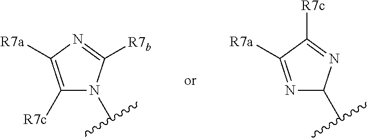

z is

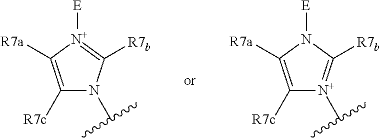

or
L is hydrogen, or methyl, or hydroxyalkyl, or deuterium;
D is deuterium (enrichment, for example, exceeding 40% deuterium incorporation at shown position, and optionally at other positions also);
S symbolises the S stereoisomer, for example, in enantiomeric excess (ee) exceeding 70%;
R₁ is cyano or —C(═O)R₉;
R₄ is halogen, C₁₋₄alkyl, trifluoromethyl, or OCF₃;

R_{7c} is hydrogen or R₇ and R_{7c} join to form a fused benzene ring optionally substituted independently with C₁₋₄alkyl or —(CH₂)₁₋₂—NHC(═O)C₁₋₄alkyl,
R_{7b} is hydrogen, C₁₋₄alkyl, or —(CH₂)₁₋₂—NHC(═O)C₁₋₄alkyl;
R₉ is a) —NR₁₀R₁₁
b) C₁₋₈ alkyl optionally substituted independently with one to two of:
i) SR₁₃, OR₁₃, NR₁₃ₐR_{13b}, halogen, trifluoromethyl, CO₂R₁₃ₐ, and C(═O)NR₁₃ₐR_{13b};
ii) cycloalkyl optionally substituted with one to two groups independently selected from C(═O)H, C₁₋₄acyl, alkenyl, carbamyl, phenyl in turn optionally substituted with halogen;
iii) phenyl or napthyl optionally substituted with one to two groups independently selected from halogen, nitro, amino, alkyl, hydroxy, C₁₋₄alkoxy, or having fused thereto a five or six membered heterocyclo;
iv) pyridinyl, thiophenyl, furanyl, tetrahydrofuranyl, or azepinyl, optionally substituted with alkyl or having fused thereto a five to six membered carbocyclic ring optionally substituted with keto and/or C₁₋₄alkoxy;
c) C1-4alkoxy;
d) C1-4alkylthio;
e) CO2alkyl;
f) 3 to 6 membered cycloalkyl optionally having up to four substituents independently selected from alkyl, halogen, cyano, alkenyl, acyl, alkylthio, carbamyl, phenyl in turn optionally substituted with halogen; or having an aryl fused thereto;
g) phenyl optionally substituted with one to four (selected independently) of halogen, cyano, trifluoromethyl, nitro, hydroxy, C₁₋₄alkoxy, haloalkoxy, C₁₋₆alkyl, CO₂alkyl, SO₂alkyl, SO₂NH₂, amino, NH(C₁₋₄alkyl), N(C₁₋₄alkyl)₂, NHC(═O)alkyl, C(═O)alkyl, C₁₋₄ alkyl in turn optionally substituted with one to three (selected independently) of trifluoromethyl, hydroxy, cyano, phenyl, pyridinyl; and/or a five or six membered heteroaryl or heterocyclo in turn optionally substituted with keto or having a benzene ring fused thereto;
h) pyridinyl, thiazolyl, furanyl, thiophenyl, and pyrrolyl optionally substituted with one to two (selected independently) of halogen, alkyl, phenyl in turn optionally substituted with halogen and/or trifluoromethyl;
R₁₀ is hydrogen, alkyl, or alkoxy;
R₁₁ is alkyl, substituted alkyl, alkoxy, heterocyclo, cycloalkyl, aryl, or heteroaryl;
or R₁₀ and R₁₁, taken together form a heterocyclo or heteroaryl;
R₂₃ is hydrogen, alkyl, hydroxyalkyl, or phenyl;
R₂₄ is (selected independently at each point of its use)alkyl, halogen, trifluoromethyl, cyano, halogen, hydroxy, OCF₃, methoxy, phenyloxy, benzyloxy, cyano, or acyl, or two R₂₄ groups join to form a fused cycloalkyl or benzene ring;
q is 0, 1, or 2;
x is 0 or 1; and
y is 0, 1, or 2.

Most preferred are compounds as immediately defined above wherein, R1 is cyano or —C(═O)R₉; R₉ is optionally substituted phenyl or phenyl C₁₋₄alkyl; x is 0 or 1; and q and y are 1 or 2. For this preferred structure, further preferred is for its L group to be methyl. Alternatively preferred is for its L group to be deuterium, wherein the S stereoisomer is preferred.

A disclosure embodiment is for L on the chiral carbon to be alkyl, or substituted alkyl (non-limiting examples: CF₃, CCl₃), or deuterated alkyl (non-limiting example: CD₃), or aminoalkyl, or thioalkyl, or alkoxy, or halogen, or haloalkyl, or haloalkoxy, or hydroxyalkyl or any atom or isotope permitted by valence (including any accompanying hydrogen(s)/deuterium(s) by valence) except hydrogen at natural abundance. A disclosure embodiment is for L on the chiral carbon to be alkyl, substituted alkyl or any atom or isotope permitted by valence except hydrogen at natural abundance. An alternative disclosure embodiment is for L to be H.

### Methyl (or Metabolized Derivative Thereof) on the Chiral Carbon

In some embodiments, a compound according to the formula

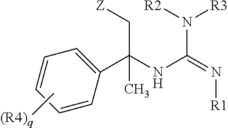

or a pharmaceutically-acceptable salt, solvate, hydrate or prodrug thereof. With a compound of this formula, in some embodiments, there is an enantiomeric excess of enantiomer. In some embodiments, the S-enantiomer of the compound is in enantiomeric excess. In other embodiments, the R-enantiomer of the compound is in enantiomeric excess. To illustrate, for (non-limiting) example, with supporting experimental data herein, against some cancers, the S-enantiomer exerts more potent anti-cancer activity and is preferred for anti-cancer use, whilst against some other cancers, the R-enantiomer exerts more potent anti-cancer activity and is preferred for anti-cancer use, wherein in some embodiments both are independently trialed against a cancer (in vivo and/or ex vivo) to see which exerts the greater anti-cancer activity, wherein administration is subsequently delimited to the enantiomer, or sample with enantiomeric excess for that enantiomer, that is found to have greater anti-cancer activity against that particular cancer, and/or the racemate or a scalemate is administered, optionally with another compound(s) of this disclosure, or a pharmaceutically-acceptable salt, solvate, hydrate or prodrug thereof.

In some embodiments, the compound is a compound according to the formula

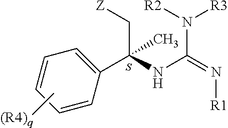

or a pharmaceutically-acceptable salt, solvate, hydrate or prodrug thereof, wherein S symbolises the S stereoisomer, for example, in enantiomeric excess (ee) exceeding 70%.

In some embodiments, the compound is a compound according to the formula

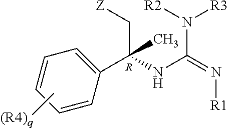

or a pharmaceutically-acceptable salt, solvate, hydrate or prodrug thereof, wherein R symbolises the R stereoisomer, for example, in enantiomeric excess (ee) exceeding 70%.

In some embodiments, the compound is a compound according to the formula

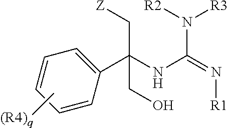

or a pharmaceutically-acceptable salt, solvate, hydrate or prodrug thereof.

In some embodiments, the compound is a compound according to the formula

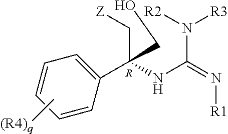

or a pharmaceutically-acceptable salt, solvate, hydrate or prodrug thereof, wherein R symbolises the R stereoisomer, for example, in enantiomeric excess (ee) exceeding 70%.

In some embodiments, the compound is

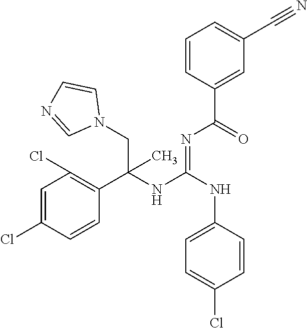 [SMILES: CC(Cn1ccnc1)(N/C(=N/C(=O)c1cccc(C#N)c1)Nc1ccc(Cl)cc1)c1ccc(Cl)cc1Cl]

or a pharmaceutically-acceptable salt, solvate, hydrate or prodrug thereof.

In some embodiments, the compound is

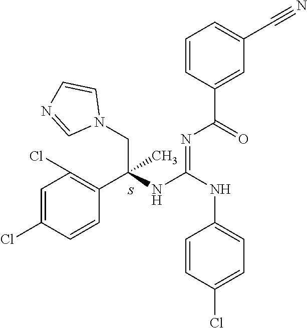

or a pharmaceutically-acceptable salt, solvate, hydrate or prodrug thereof; in some embodiments the enantiomeric excess (ee) of the S stereoisomer exceeds 70%.

In some embodiments, the compound is

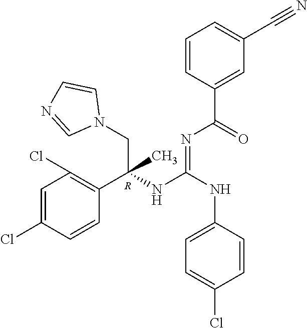

or a pharmaceutically-acceptable salt, solvate, hydrate or prodrug thereof; in some embodiments the enantiomeric excess (ee) of the R stereoisomer exceeds 70%.

In some embodiments, the compound is

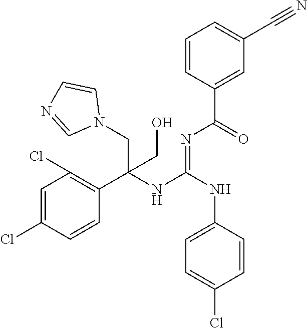 [SMILES: N#Cc1cccc(C(=O)/N=C(\Nc2ccc(Cl)cc2)NC(CO)(Cn2ccnc2)c2ccc(Cl)cc2Cl)c1]

or a pharmaceutically-acceptable salt, solvate, hydrate or prodrug thereof.

In some embodiments, the compound is

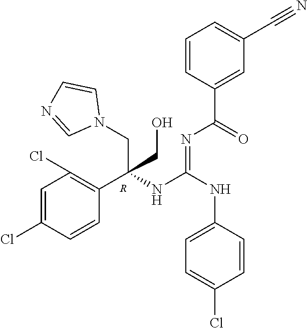

or a pharmaceutically-acceptable salt, solvate, hydrate or prodrug thereof; in some embodiments the enantiomeric excess (ee) of the R stereoisomer exceeds 70%;
optionally wherein there is an enantiomeric excess (ee) of the S stereoisomer instead, optionally exceeding 70%.
   Some example embodiments of Formula (I)

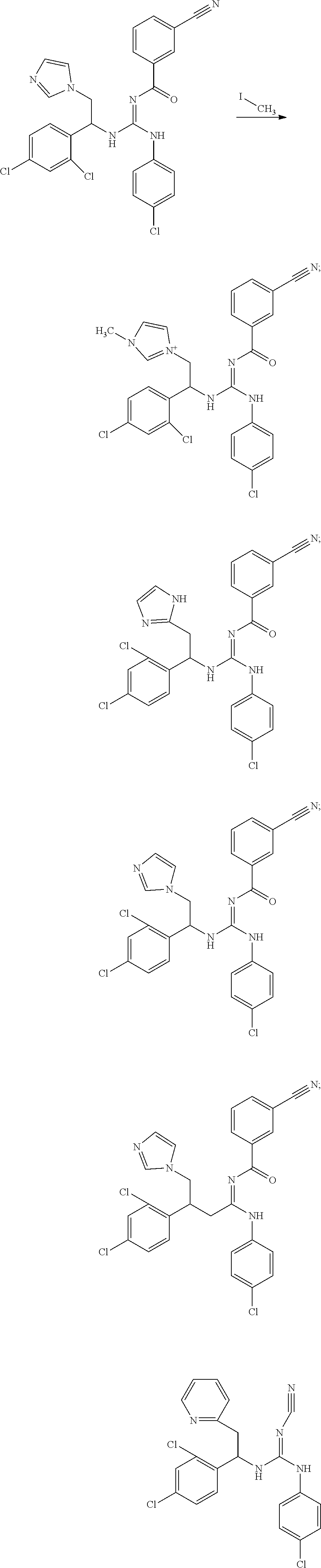

### Specific Compound Synthesis

Starting reagents for following synthesis were sourced commercially using the LabNetwork (www.labnetwork.com), which is a website that permits one to search for chemical suppliers for inputted structures/chemical names. There are numerous suppliers listed on LabNetwork for the starting compound, Compound 1 (e.g. Apollo Scientific Ltd., Stockport, UK), and for Compound 3-A (e.g. Astatech Inc., Bristol PA, USA) and Compound 5-A (e.g. Atlantic Research Chemicals Ltd., Bude, UK).

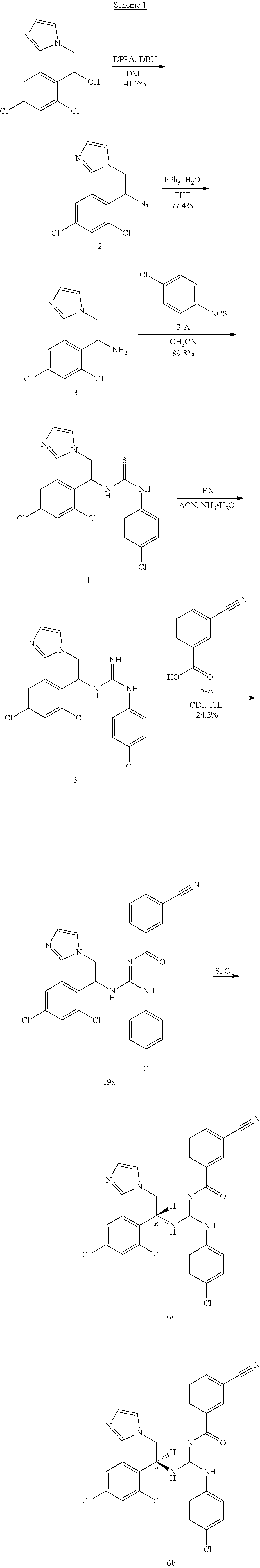

Products of Scheme 1: Stereoisomer (“Stereoisomer 1”, enantiomeric excess >97%): Liquid Chromatography-Mass Spectrometry (LC-MS): Liquid chromatography (LC) retention time (RT)=2.516 minutes, Mass spectrometry (MS; electrospray ionization, positive mode): m/z 537.1 [M+H]⁺, 559.1 [M+Na]⁺, 269.1 [M+2H]²⁺. ¹H NMR (400 MHZ, DMSO-d6) δ (ppm) 11.43 (s, 1H), 8.30 (s, 1H), 8.21 (d, J=7.9 Hz, 1H), 7.93 (d, J=7.8 Hz, 2H), 7.68-7.56 (m, 3H), 7.56-7.46 (m, 4H), 7.20 (s, 1H), 7.09 (d, J=8.1 Hz, 2H), 6.97 (s, 1H), 5.96 (s, 1H), 4.33 (s, 1H), 4.19 (s, 1H). {NMR probe temperature=298.15 K}. Opposite stereoisomer (“Stereoisomer 2”, enantiomeric excess >97%): LC-MS: Liquid Chromatography RT=2.516 minutes, Mass spectrometry (MS; electrospray ionization, positive mode): m/z 537.1 [M+H]⁺, 559.1 [M+Na]⁺, 269.1 [M+2H]²⁺. ¹H NMR (400 MHZ, DMSO-d6) δ (ppm) 11.43 (s, 1H), 8.30 (s, 1H), 8.21 (d, J=7.9 Hz, 1H), 7.93 (d, J=7.8 Hz, 2H), 7.67-7.56 (m, 3H), 7.50 (t, J=6.5 Hz, 4H), 7.20 (s, 1H), 7.09 (d, J=8.2 Hz, 2H), 6.97 (s, 1H), 5.96 (s, 1H), 4.34 (s, 1H), 4.19 (s, 1H). {NMR probe temperature=298.15 K}.

The independent reaction schemes below, Scheme 2 and Scheme 3, are each the same as Scheme 1 up until Compound 5. This shared component isn't shown, just their point of divergence from Compound 5, which comes from using a different Compound 5-A, each of which is available from multiple suppliers on www.labnetwork.com (e.g. HE Chemical, Changzhou, Jiangsu, China).

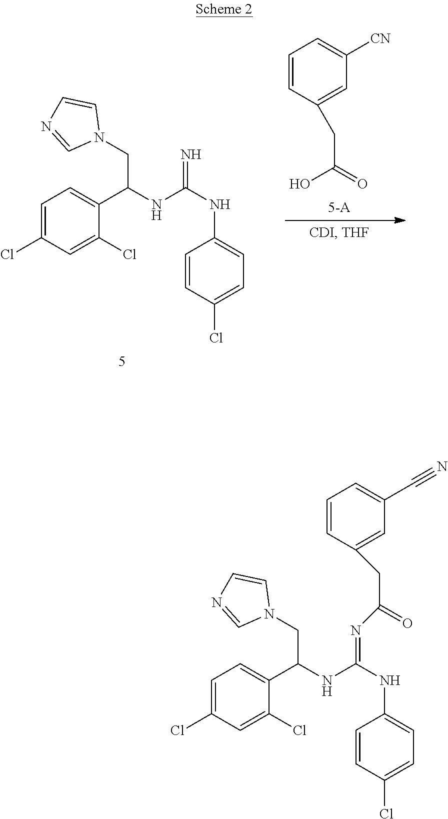 [SMILES: N=C(Nc1ccc(Cl)cc1)NC(Cn1ccnc1)c1ccc(Cl)cc1Cl ; N#Cc1cccc(CC(=O)O)c1 ; N#Cc1cccc(CC(=O)/N=C(\Nc2ccc(Cl)cc2)NC(Cn2ccnc2)c2ccc(Cl)cc2Cl)c1]

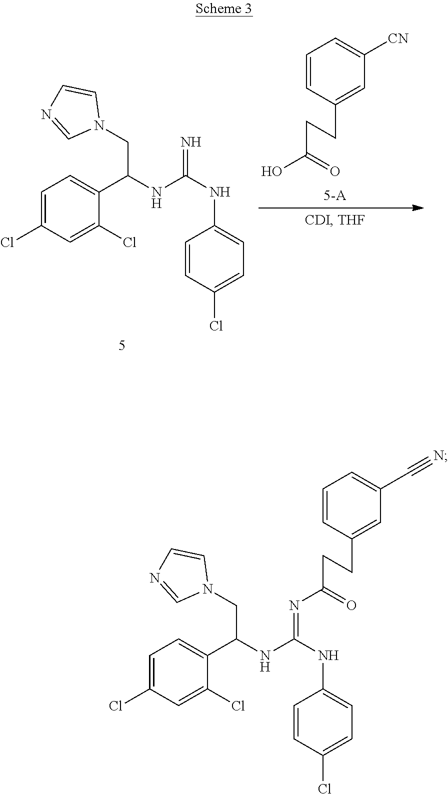 [SMILES: N=C(Nc1ccc(Cl)cc1)NC(Cn1ccnc1)c1ccc(Cl)cc1Cl ; N#Cc1cccc(CCC(=O)O)c1 ; N#Cc1cccc(CCC(=O)/N=C(\Nc2ccc(Cl)cc2)NC(Cn2ccnc2)c2ccc(Cl)cc2Cl)c1]

The reaction schemes below, Scheme 4 and Scheme 5, show only starting material and product because they use the same internal steps as Scheme 1 (not shown), but with a different starting material, a different Compound 1 than Scheme 1 and each other, as shown below, and thence different products produced, as shown. Compound 1 of Scheme 4 and Scheme 5 are available from suppliers listed on www.labnetwork.com (e.g. Toronto Research Chemicals, Ontario, Canada).

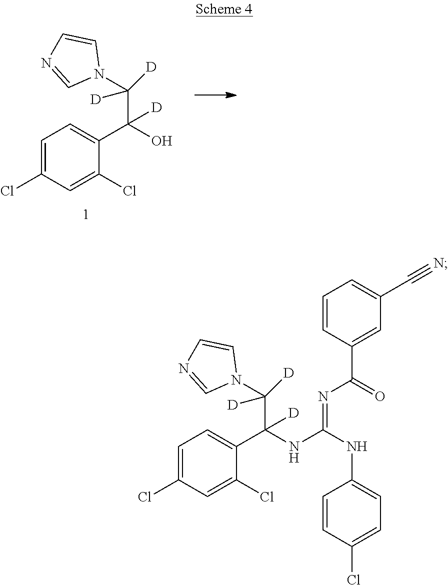 [SMILES: [2H]C(O)(c1ccc(Cl)cc1Cl)C([2H])([2H])n1ccnc1 ; [2H]C(N/C(=N/C(=O)c1cccc(C#N)c1)Nc1ccc(Cl)cc1)(c1ccc(Cl)cc1Cl)C([2H])([2H])n1ccnc1]

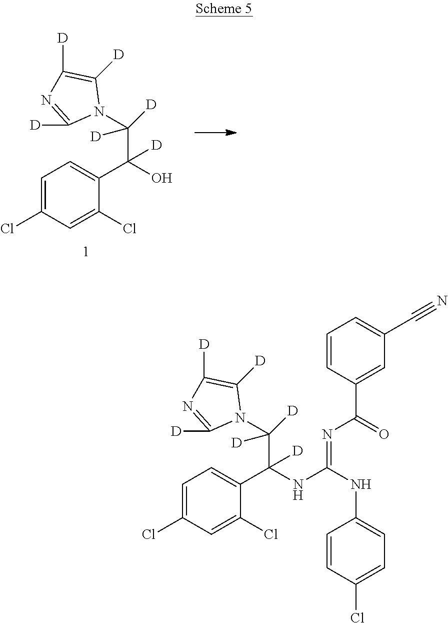 [SMILES: [2H]c1nc([2H])n(C([2H])([2H])C([2H])(O)c2ccc(Cl)cc2Cl)c1[2H] ; [2H]c1nc([2H])n(C([2H])([2H])C([2H])(N/C(=N/C(=O)c2cccc(C#N)c2)Nc2ccc(Cl)cc2)c2ccc(Cl)cc2Cl)c1[2H]]

The reaction scheme below, Scheme 6, is modified from Scheme 1, in order to produce a deuterated analogue, with deuterium in place of hydrogen on the chiral carbon. The starting compound in the scheme below, Compound 1, is available from multiple suppliers listed on LabNetwork (e.g. Apollo Scientific Ltd., Stockport, UK). The 2^{nd} compound, Compound 2, is also available from multiple suppliers listed on LabNetwork (e.g. Manchester Organics Ltd., UK). So, one of the art can choose between these two starting options. In an alternative embodiment (not shown), Compound 1A is replaced with Imidazole-¹³C,¹⁵N₂ (CAS no: 1173018-62-6; available from suppliers on www.Labnetwork.com e.g. Meihezhiku (Wuhan) Biotechnology Co., Ltd, China) in Scheme 6 to produce an alternative product of this disclosure, isotopically enriched for ¹³C and ¹⁵N at equivalent positions in the final product.

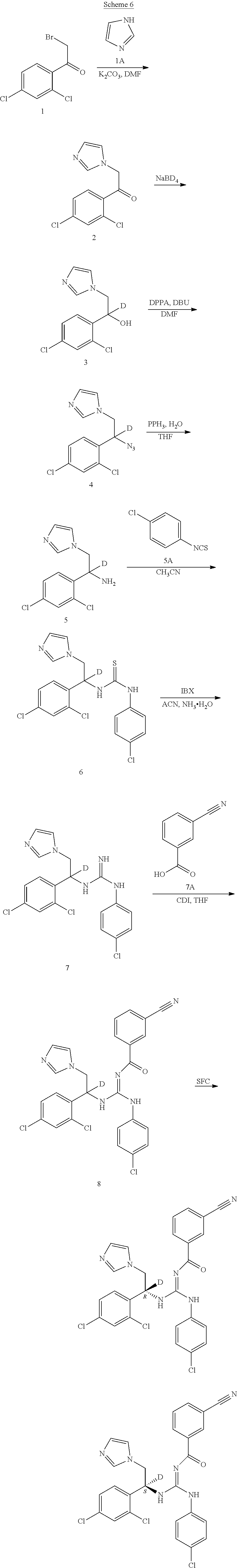

Products of Scheme 6: Stereoisomer (“Stereoisomer A”, enantiomeric excess >97%): LC-MS: LC retention time (RT)=2.685 minutes, MS (electrospray ionization, positive mode): m/z 538.1 [M+H]⁺, 560.1 [M+Na]⁺, 269.6 [M+2H]²⁺. High Resolution Mass Spectrometry (HRMS): Liquid Chromatography-Time of Flight (LC-TOF) MS (electrospray ionization, positive mode): LC Retention Time (RT)=0.166 minutes, m/z 538.0745928061 [M+H]⁺, m/z 560.0600137508 [M+Na]⁺, m/z 576.0250917093 [M+K]⁺, molar percent deuterium incorporation at chiral carbon=99.13%. ¹H NMR (400 MHZ, DMSO-d6) δ 11.48 (s, 1H), 8.33 (s, 1H), 8.25 (dt, J=7.8, 1.5 Hz, 1H), 7.96 (dt, J=7.7, 1.5 Hz, 1H), 7.92 (s, 1H), 7.71-7.60 (m, 3H), 7.60-7.49 (m, 4H), 7.23 (s, 1H), 7.14 (s, 2H), 7.00 (s, 1H), 4.38 (d, J=14.1 Hz, 1H), 4.23 (s, 1H). {NMR probe temperature=301 K}. Opposite stereoisomer (“Stereoisomer B”, enantiomeric excess >97%): LC-MS: LC retention time (RT)=2.685 minutes, MS (electrospray ionization, positive mode): m/z 538.1 [M+H]⁺, 560.1 [M+Na]⁺, 269.6 [M+2H]²⁺. HRMS: LC-TOF MS (electrospray ionization, positive mode): LC RT=0.163 minutes, m/z 538.0727757864 [M+H]⁺, m/z 560.0513502753 [M+Na]⁺, m/z 576.0327248583 [M+K]⁺, molar percent deuterium incorporation at chiral carbon=99.14%. ¹H NMR (400 MHZ, DMSO-d6) δ11.46 (s, 1H), 8.32 (s, 1H), 8.24 (d, J=7.9 Hz, 1H), 7.95 (d, J=7.7 Hz, 2H), 7.70-7.59 (m, 3H), 7.59-7.49 (m, 4H), 7.21 (s, 1H), 7.12 (d, J=7.9 Hz, 2H), 6.99 (s, 1H), 4.36 (d, J=13.9 Hz, 1H), 4.22 (s, 1H). {NMR probe temperature=300.7 K}.

The reaction scheme below, Scheme 8, is the same as Scheme 6 up until Compound 7. This shared component isn't shown, just the point of divergence from Compound 7, which comes from using a different Compound 7A, which is available from multiple suppliers on www.labnetwork.com (e.g. HE Chemical, Changzhou, Jiangsu, China).

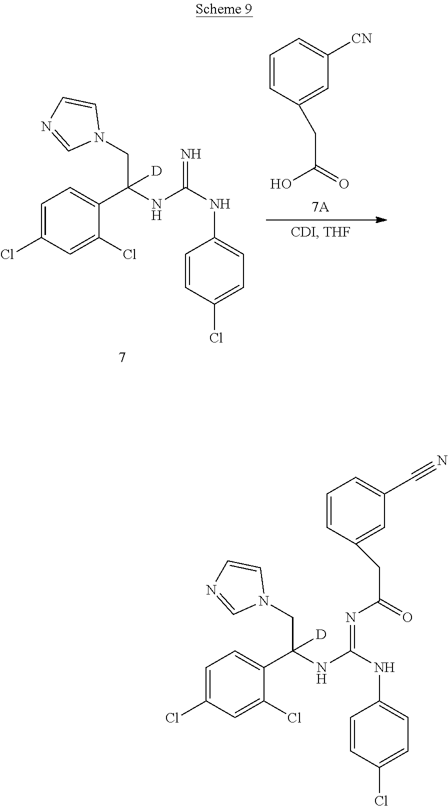 [SMILES: [2H]C(Cn1ccnc1)(NC(=N)Nc1ccc(Cl)cc1)c1ccc(Cl)cc1Cl ; N#Cc1cccc(CC(=O)O)c1 ; [2H]C(Cn1ccnc1)(N/C(=N/C(=O)Cc1cccc(C#N)c1)Nc1ccc(Cl)cc1)c1ccc(Cl)cc1Cl]

The reaction scheme below, Scheme 9, is the same as Scheme 6 up until Compound 7. This shared component isn't shown, just the point of divergence from Compound 7, which comes from using a different Compound 7A, wherein three different options of Compound 7A are shown and the three resultant products, wherein Compound 7A(i), 7A(ii) and 7A(iii) are all available from multiple suppliers on www.labnetwork.com (e.g. all available from Fluorochem, Hadfield, Derbyshire, UK).

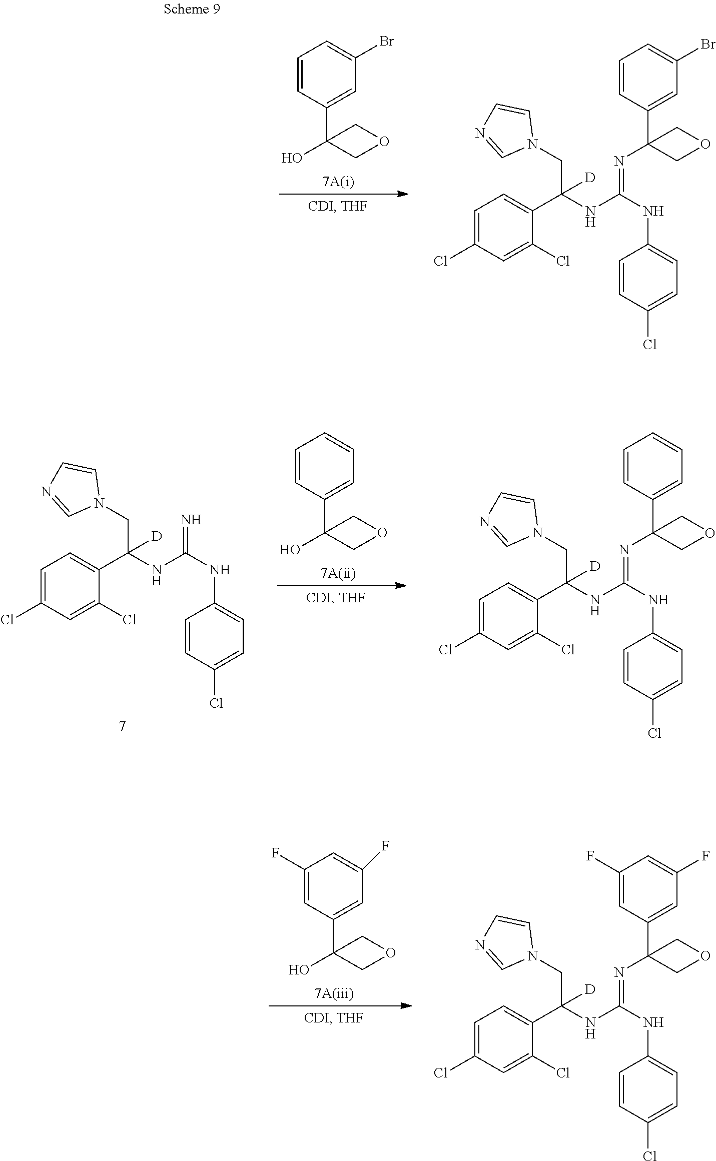

The reaction scheme below, Scheme 10, shows only starting material and product because it uses the same internal steps as Scheme 6, but with a different starting compound, as shown below (available from suppliers listed on www.labnetwork.com e.g. Matrix Scientific, Columbia, SC, USA), and thence a different product is produced, as shown. The other reaction scheme immediately below, Scheme 11, shows only starting material and product because it uses the same internal steps as Scheme 6 (starting from its Compound 2), but with a different starting compound, as shown below (available from suppliers listed on www.labnetwork.com e.g. Vitas-M Laboratory, Champaign, IL, USA), and thence a different product is produced, as shown. Product shown is the (predicted, MarvinSketch software [Chemaxon, Hungary]) predominant tautomer.

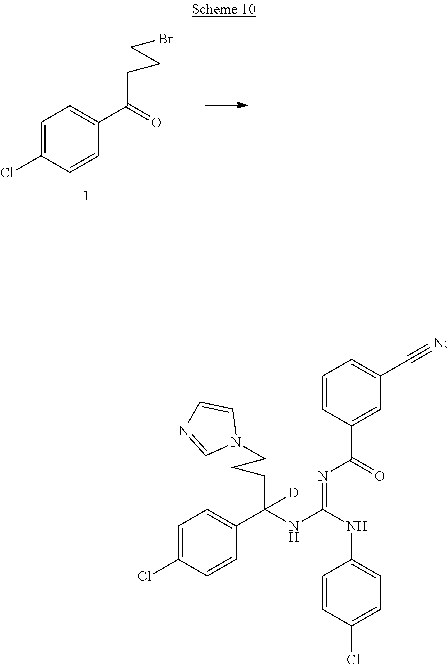 [SMILES: O=C(CCCBr)c1ccc(Cl)cc1 ; [2H]C(CCCn1ccnc1)(N/C(=N/C(=O)c1cccc(C#N)c1)Nc1ccc(Cl)cc1)c1ccc(Cl)cc1]

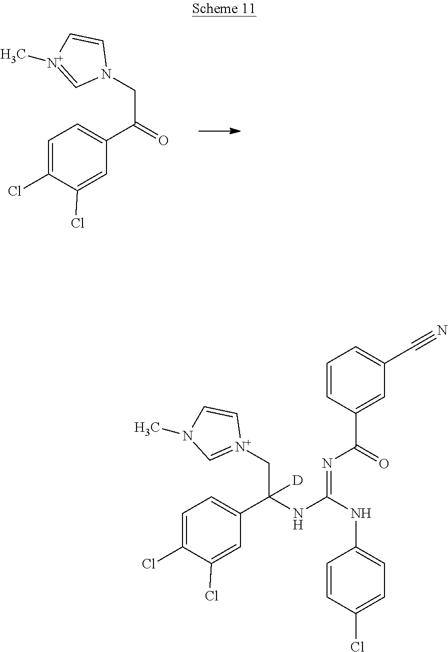 [SMILES: C[n+]1ccn(CC(=O)c2ccc(Cl)c(Cl)c2)c1 ; [2H]C(C[n+]1ccn(C)c1)(N/C(=N/C(=O)c1cccc(C#N)c1)Nc1ccc(Cl)cc1)c1ccc(Cl)c(Cl)c1]

Using NaBT₄, where T is tritium (3H), instead of NaBD₄ in Scheme 6 produces final product (Compound 8) with tritium instead of deuterium on the chiral carbon, wherein this synthetic route and its tritiated product (and intermediates) are componentry to the present disclosure. The tritiated form of Compound 5 in this tritium variant of Scheme 6 can be substituted into the synthesis schemes described in [P1] to produce tritiated molecules, with tritium on their chiral carbon, which are componentry to the present disclosure, and in a non-limiting embodiment, one of more of these new compositions of matter are used as anti-cancer medicines.

Using NaBT₄ and NaBD₄ in Scheme 6 produces final compound (Compound 8) enriched for tritium (T) and deuterium (D) at the chiral carbon. Wherein the relative amount of enrichment can be set by setting the relative amount of NaBT₄ and NaBD₄ used. Wherein it is harder to enrich for tritium than deuterium. So, for example, if equal enrichment is sought then more NaBT₄ than NaBD₄ is required. One of the art, by routine experimentation, can modulate the relative amounts of NaBT₄ and NaBD₄ to confer their desired relative amount of enrichment. Wherein this synthetic route, its product and intermediates are componentry to the present disclosure. The tritium and deuterium enriched form of Compound 5 from this NaBT₄ and NaBD₄ variant of Scheme 6 can be substituted into the synthesis schemes described in [P1] to produce tritium and deuterium enriched compounds, which are componentry to the present disclosure, and in a non-limiting embodiment, one of more of these new compositions of matter are used as anti-cancer medicines. In an embodiment, deuterium is enriched more. In another embodiment, tritium is enriched more. In an alternative embodiment, deuterium and tritium are enriched equally (in sense that they have equal relative abundance as compared to protium, ¹H, wherein in this situation tritium could actually be said to be enriched more because it has a lower natural {so starting} abundance).

The reaction scheme below, Scheme 12, differs from Scheme 1 in order to produce methylated analogues, with methyl in place of hydrogen on the chiral carbon. The starting compound and imidazole are both available from Apollo Scientific Ltd., Stockport, UK and other suppliers listed on www.labnetwork.com. In an alternative embodiment (not shown), Compound 1A is replaced with Imidazole-¹³C,¹⁵N₂ (CAS no: 1173018-62-6; available from suppliers on Labnetwork e.g. Meihezhiku (Wuhan) Biotechnology Co., Ltd, China) in Scheme 12 to produce an alternative product of this disclosure, isotopically enriched for ¹³C and ¹⁵N at equivalent positions in the final product.

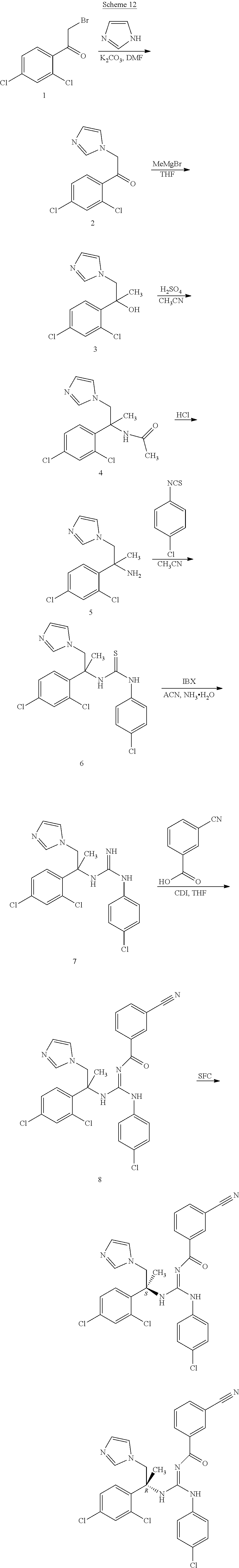

Products of Scheme 12: Stereoisomer (“Stereoisomer α”, enantiomeric excess >97%): LC retention time (RT)=2.536 minutes, MS (electrospray ionization, positive mode): m/z 551.0 [M+H]⁺, 573.0 [M+Na]⁺, 276.0 [M+2H]²⁺. ¹H NMR (400 MHZ, DMSO-d6) δ (ppm) 11.37 (s, 1H), 7.89 (dt, J=7.6, 1.5 Hz, 1H), 7.80 (d, J=7.8 Hz, 1H), 7.65-7.56 (m, 3H), 7.55-7.47 (m, 2H), 7.47-7.37 (m, 3H), 7.22 (d, J=8.4 Hz, 2H), 7.05 (s, 1H), 6.95 (s, 1H), 6.91 (s, 1H), 4.88 (d, J=13.6 Hz, 1H), 4.76 (d, J=13.7 Hz, 1H), 1.70 (s, 3H). {NMR probe temperature=298.2 K}. Opposite Stereoisomer (“Stereoisomer B”, enantiomeric excess >97%): LC retention time (RT)=2.540 minutes, MS (electrospray ionization, positive mode): m/z 551.1 [M+H]⁺, 573.0 [M+Na]⁺, 276.1 [M+2H]²⁺;

In some embodiments, Scheme 12 is implemented with each intermediate purified by HPLC, especially the last 3 intermediates, wherein this yields a lower fraction of impurit[y/ies] in the final product. Two alternatives for a step in Scheme 12:

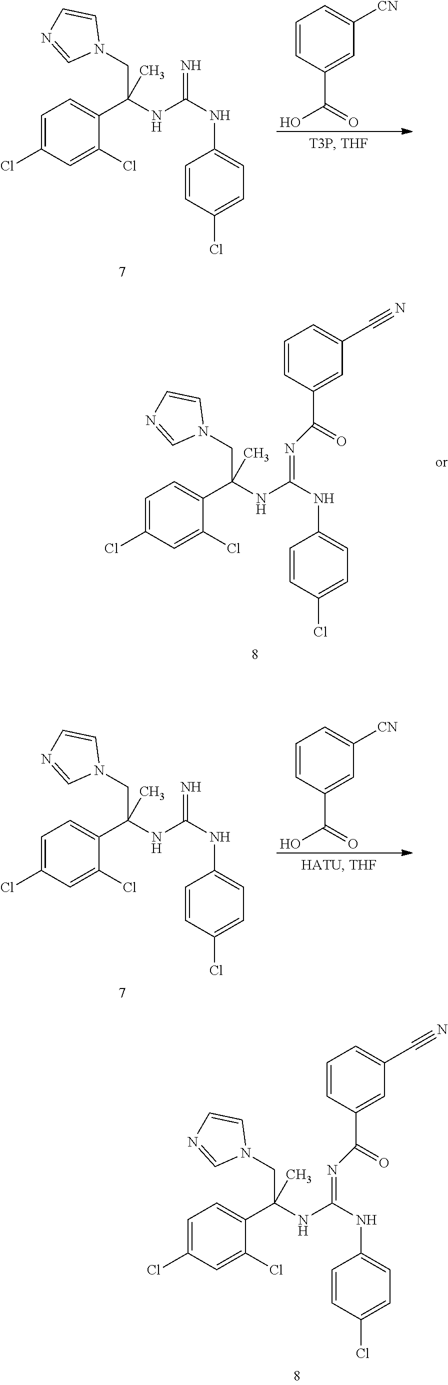 [SMILES: CC(Cn1ccnc1)(NC(=N)Nc1ccc(Cl)cc1)c1ccc(Cl)cc1Cl ; N#Cc1cccc(C(=O)O)c1 ; CC(Cn1ccnc1)(N/C(=N/C(=O)c1cccc(C#N)c1)Nc1ccc(Cl)cc1)c1ccc(Cl)cc1Cl ; CC(Cn1ccnc1)(NC(=N)Nc1ccc(Cl)cc1)c1ccc(Cl)cc1Cl ; N#Cc1cccc(C(=O)O)c1 ; CC(Cn1ccnc1)(N/C(=N/C(=O)c1cccc(C#N)c1)Nc1ccc(Cl)cc1)c1ccc(Cl)cc1Cl]

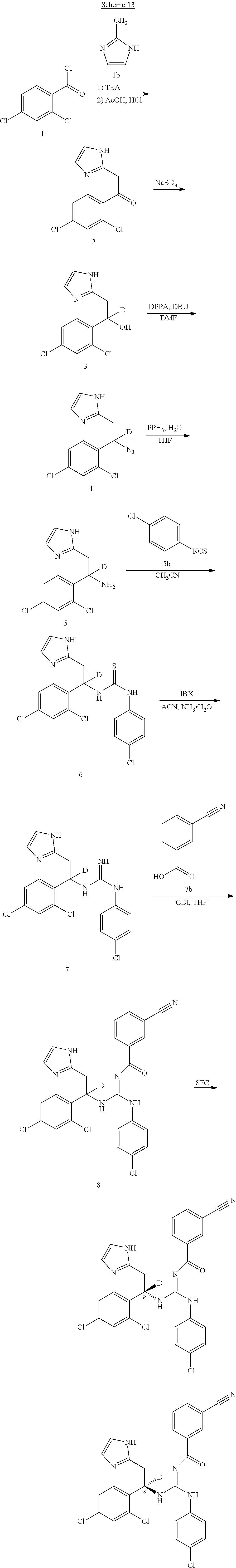

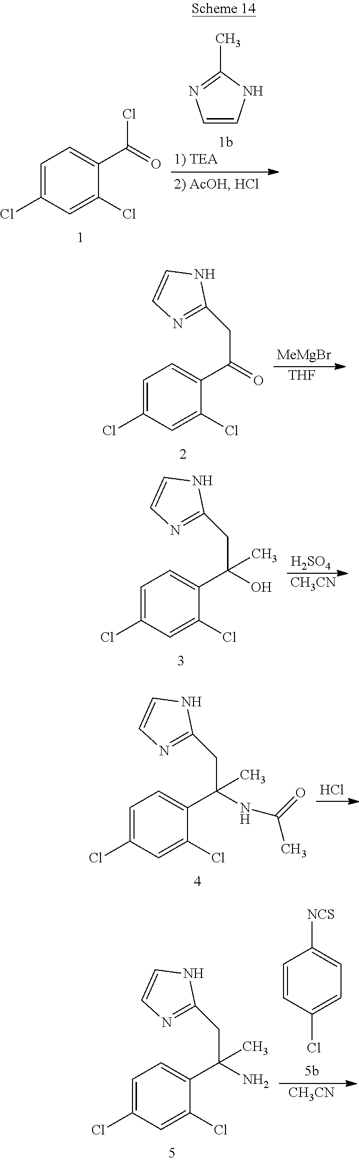

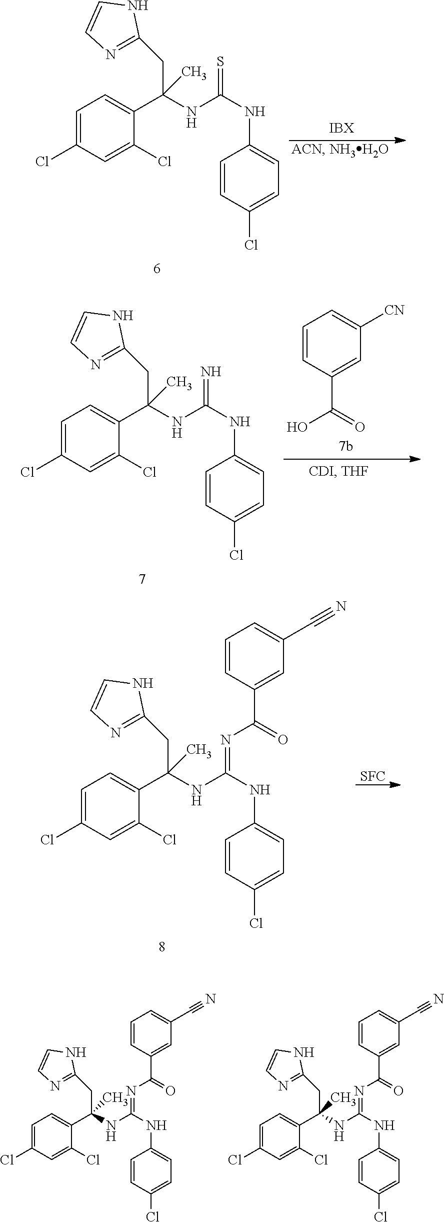

For Scheme 13 and Scheme 14 above, starting materials are available from suppliers listed on www.labnetwork.com. For example: Compound 1 (e.g. Apollo Scientific Ltd., Stockport, UK), Compound 1b (e.g. ChemScene, Monmouth Junction, NJ, USA), Compound 5b (e.g. Astatech Inc., Bristol PA, USA) and Compound 7b (e.g. Atlantic Research Chemicals Ltd., Bude, UK). For Scheme 15 below, starting materials are available from suppliers listed on www.labnetwork.com: e.g. Compound 1b from ChemScene, Monmouth Junction, NJ, USA; e.g. Compound 5b from HE Chemical, Changzhou, Jiangsu, China.

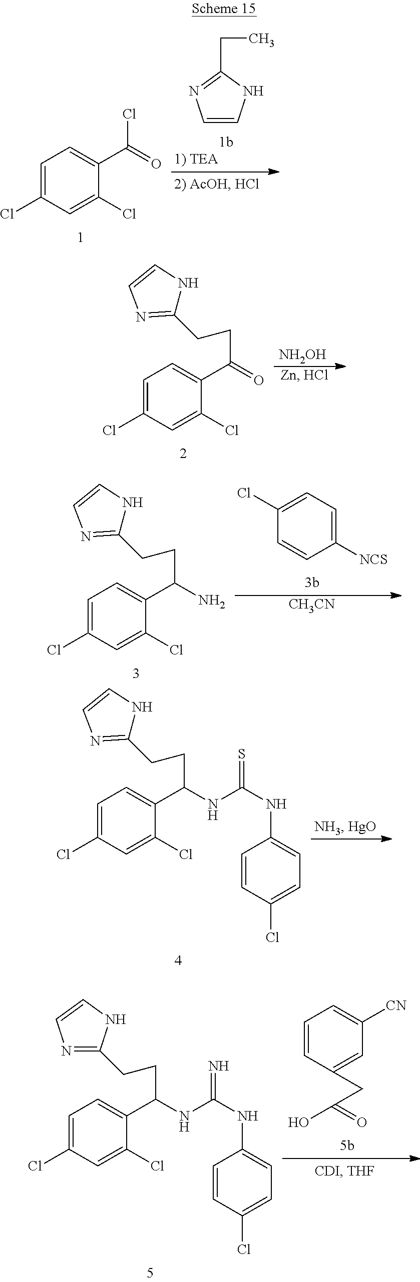

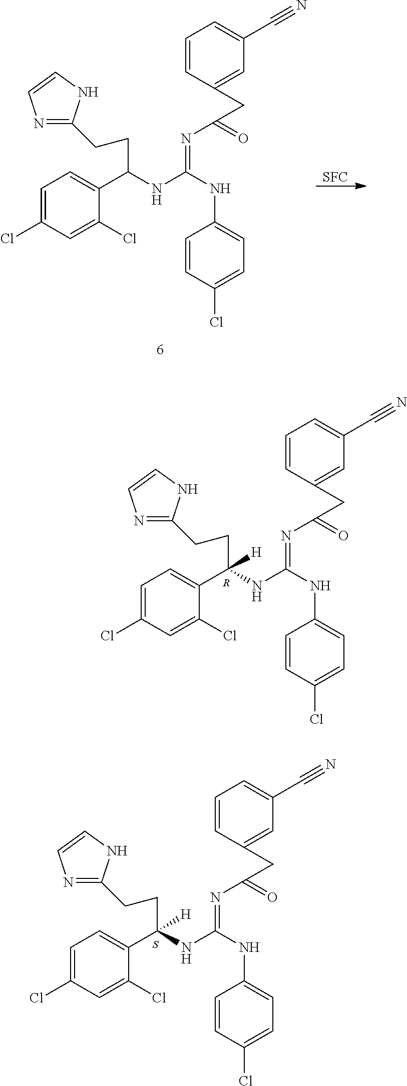

For Scheme 16 below, starting materials are commerically available from suppliers listed on www.labnetwork.com: e.g. Compound 1 (Toronto Research Chemicals, Ontario, Canada), Compound 4 and Compound 7 (Astatech Inc., Bristol PA, USA).

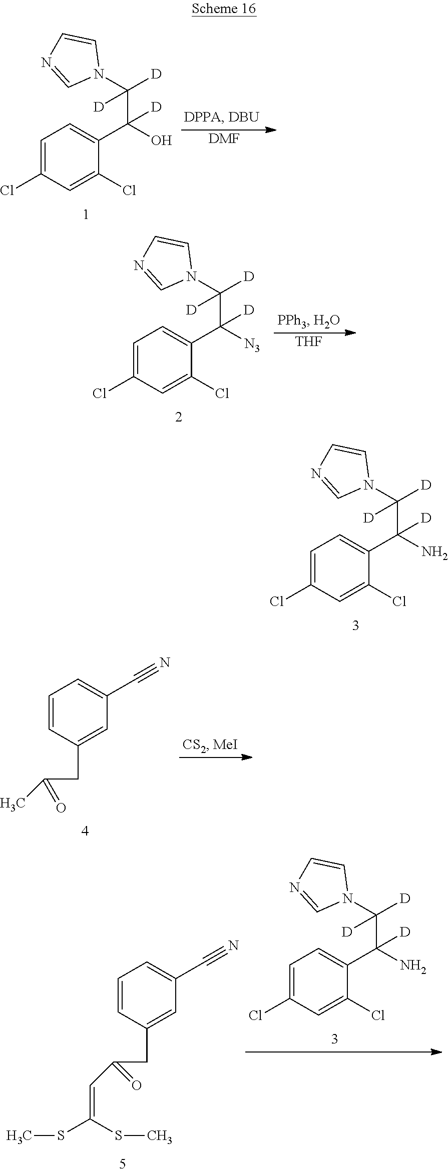

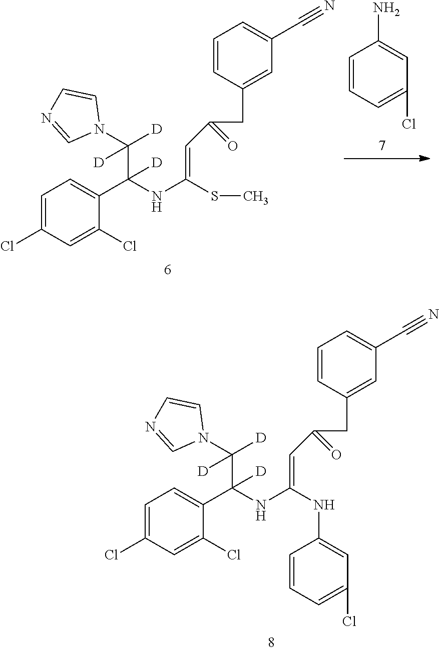 [SMILES: [2H]C(N/C(=C/C(=O)Cc1cccc(C#N)c1)SC)(c1ccc(Cl)cc1Cl)C([2H])([2H])n1ccnc1 ; Nc1cccc(Cl)c1 ; [2H]C(N/C(=C/C(=O)Cc1cccc(C#N)c1)Nc1cccc(Cl)c1)(c1ccc(Cl)cc1Cl)C([2H])([2H])n1ccnc1]

For Scheme 17 below starting materials are commerically available from suppliers listed on www.labnetwork.com: e.g. Compounds 1, 3, 9, 11 (Toronto Research Chemicals, Ontario, Canada).

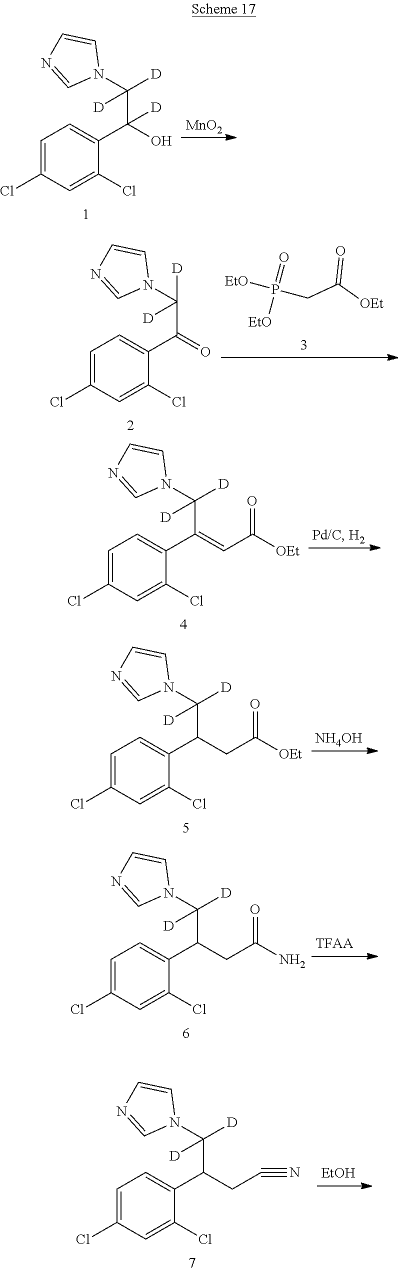

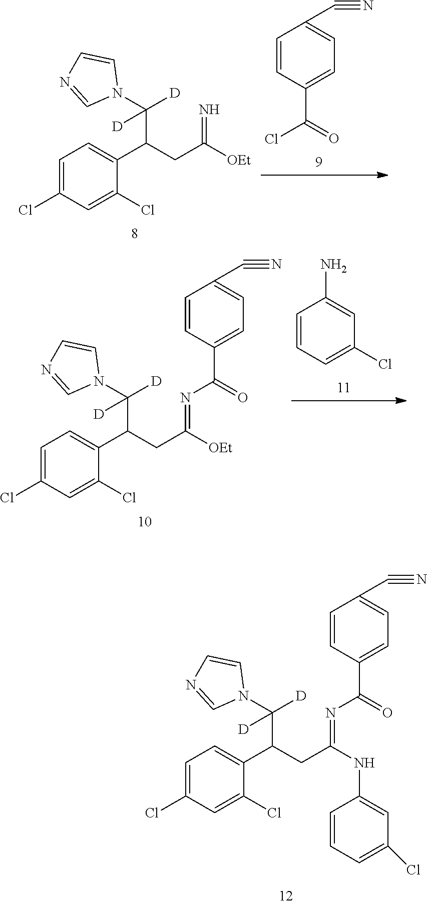 [SMILES: [2H]C([2H])(C(CC(=N)OCC)c1ccc(Cl)cc1Cl)n1ccnc1 ; N#Cc1ccc(C(=O)Cl)cc1 ; [2H]C([2H])(C(C/C(=N/C(=O)c1ccc(C#N)cc1)OCC)c1ccc(Cl)cc1Cl)n1ccnc1 ; Nc1cccc(Cl)c1 ; [2H]C([2H])(C(C/C(=N/C(=O)c1ccc(C#N)cc1)Nc1cccc(Cl)c1)c1ccc(Cl)cc1Cl)n1ccnc1]

For Scheme 18 below, starting compounds can be sourced from suppliers listed on www.labnetwork.com, e.g. Compounds 1 and 8 can be sourced from Apollo Scientific Ltd., Stockport, UK, Compounds 3, 11 and 14 can be sourced from Astatech Inc., Bristol PA, USA.

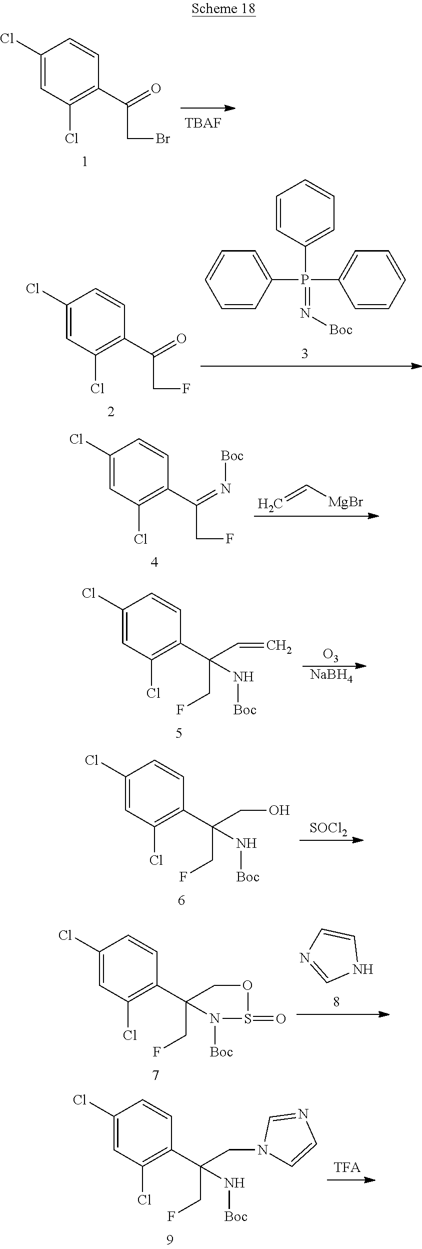

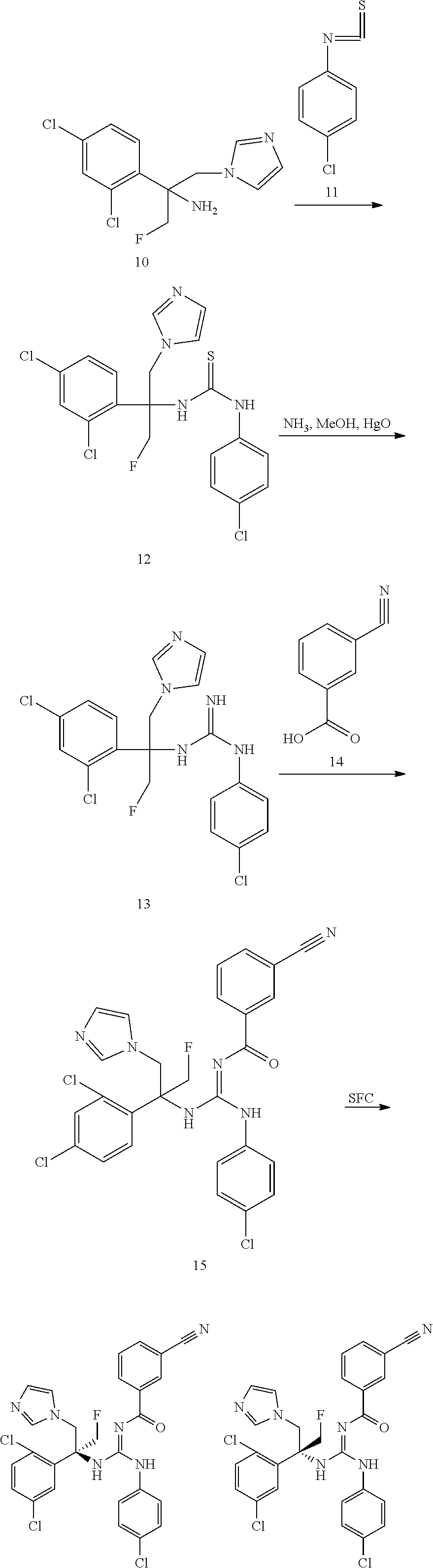

For Scheme 19 below, starting compounds can be sourced from suppliers listed on www.labnetwork.com, e.g. Compounds 1, 5, 7, 14 can be sourced from Astatech Inc., Bristol PA, USA and Compound 3 from Apollo Scientific Ltd., Stockport, UK.

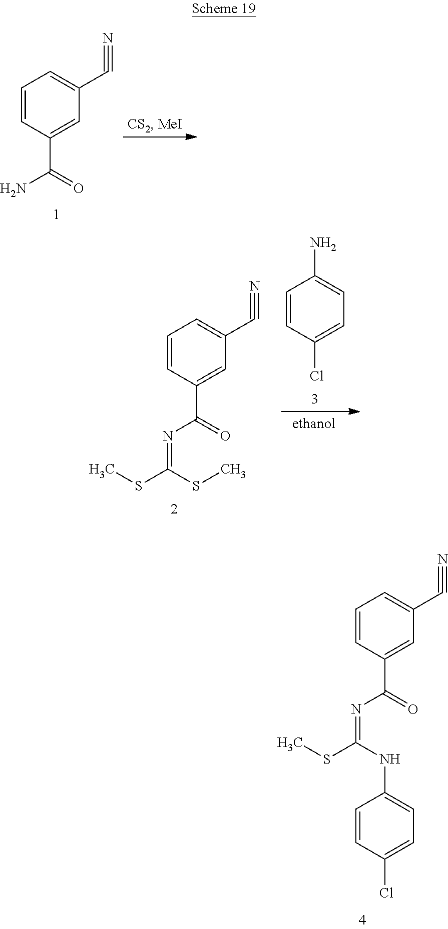

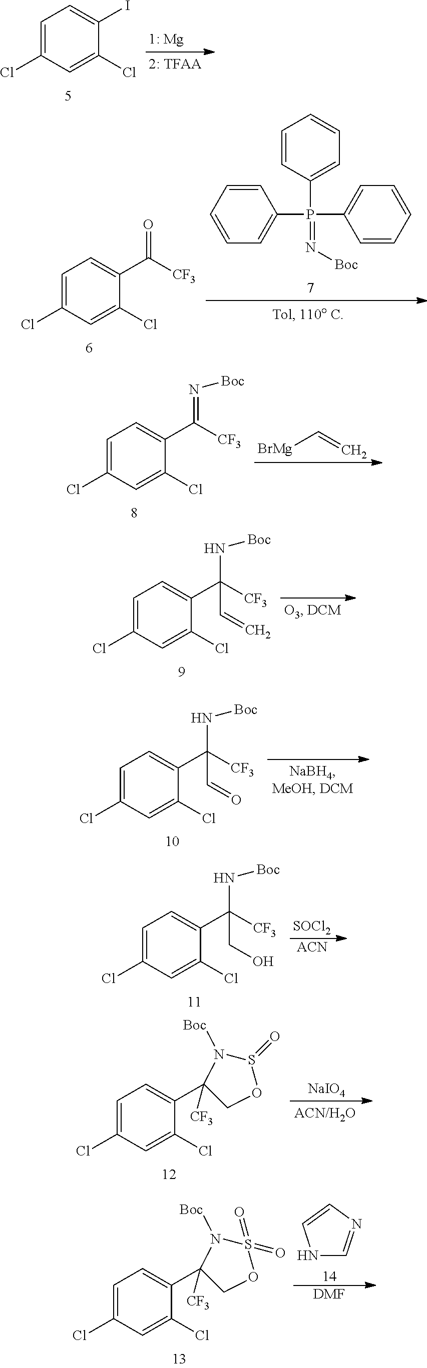

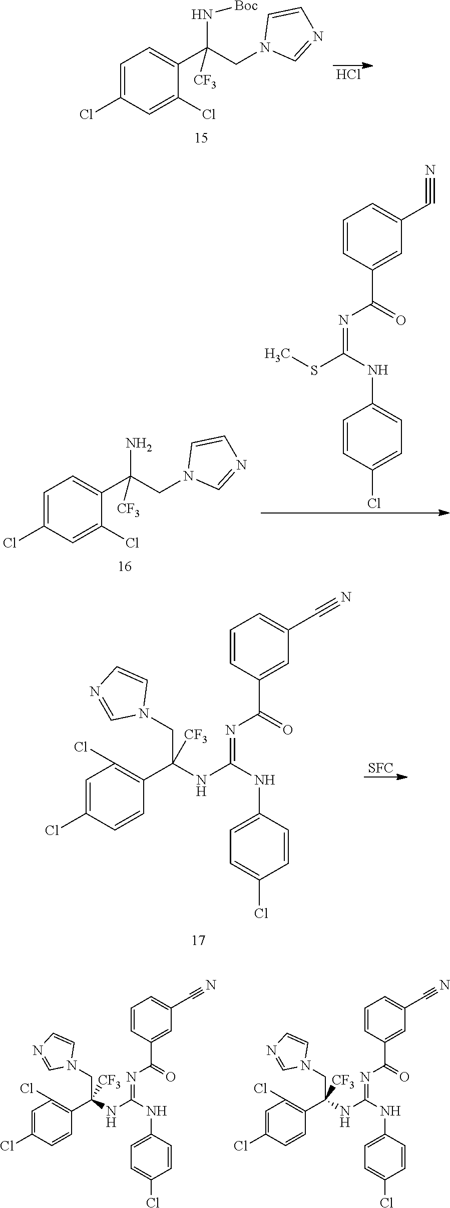

For Scheme 20 below, wherein its starting Compound 16 is from Scheme 19 above, other inputted compounds can be sourced from suppliers listed on www.labnetwork.com, e.g. Compounds 17 and 18 can be sourced from Astatech Inc., Bristol PA, USA.

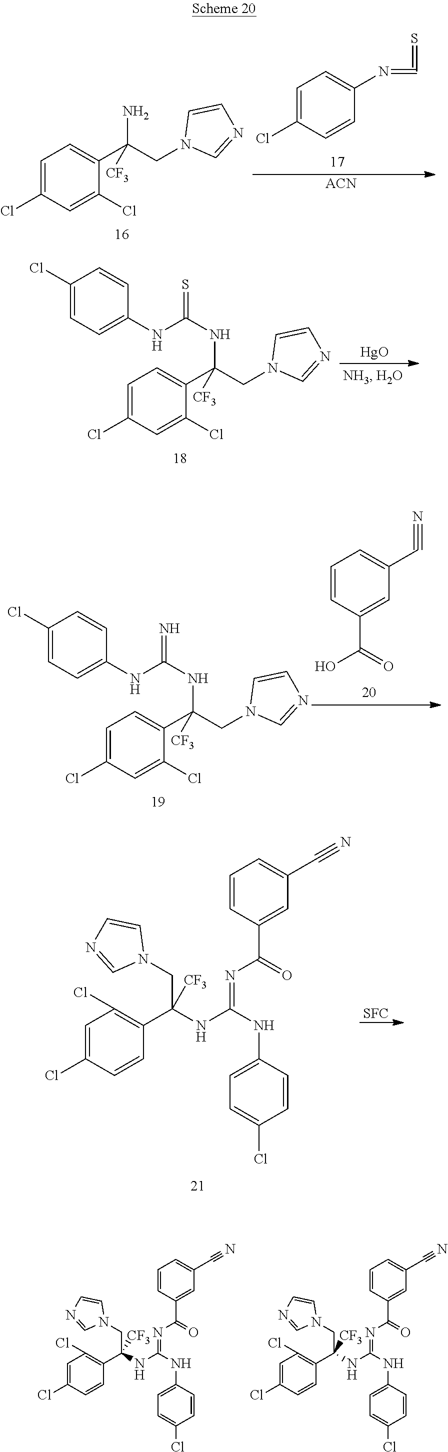

Compound 16 in Scheme 20 above is of the form of Compound 1, the starting compound, in the molecule synthesis embodiments of [P1] (presented in its “Process of Preparation” section), BUT with the exception that has CF₃ on its chiral carbon. This trifluoromethylated form can be substituted into the synthesis schemes described in [P1] to produce trifluoromethylated molecules, with CF₃ on their chiral carbon, that are componentry to the present disclosure, and in a non-limiting embodiment, one of more of these new compositions of matter are used as anti-cancer medicines. Solvents, temperatures, pressures, and other reaction conditions may readily be selected by one of ordinary skill in the art. Starting materials are commercially available or can be readily prepared by one of ordinary skill in the art using known methods.

### Example (II)

### Summary of Formula (II)

This disclosure embodiment relates to compounds having the formula: Formula (II)

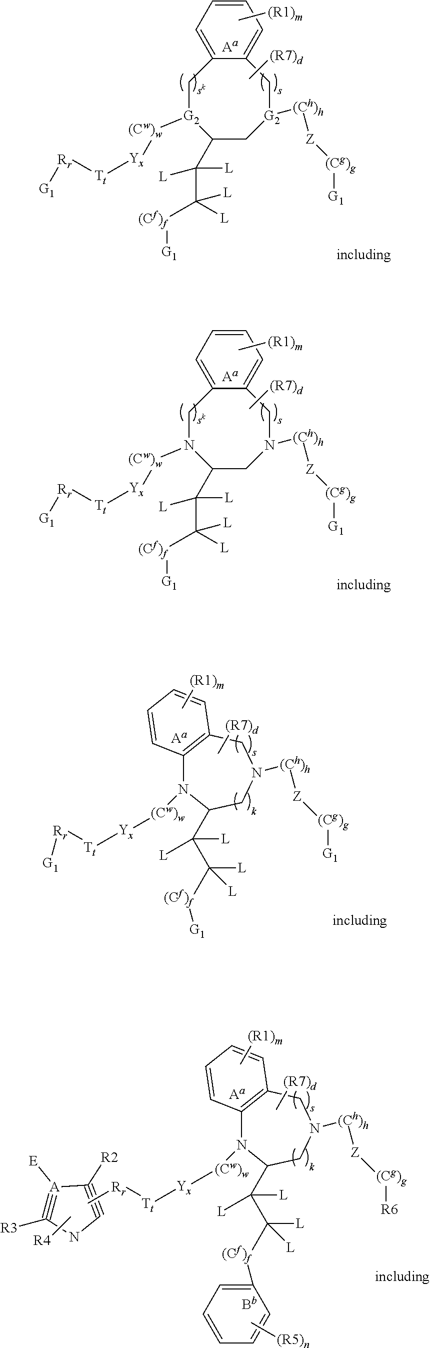

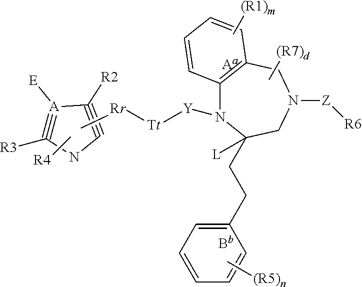

or pharmaceutically-acceptable salts, solvates, hydrates or prodrugs thereof, wherein:
G₁ is, independently at each point of use, aryl or heteroaryl;
G₂ is, independently at each point of use, N or CH;
L is independently at each point of use alkyl, or substituted alkyl, or deuterated alkyl, or aminoalkyl, or thioalkyl, or alkoxy, or halogen, or haloalkyl, or haloalkoxy, or hydroxyalkyl, or any atom or isotope permitted by valence (including any accompanying hydrogens by valence e.g. (non-limiting) OH, NH₂, SH, SiH₃, PH₂ etc.), for example hydrogen, or deuterium, or fluorine;
A is nitrogen (N), or N⁺, or carbon;
E is absent, or alkyl, or substituted alkyl, or deuterated alkyl, or aminoalkyl, or thioalkyl, or alkoxy or any atom or isotope permitted by valence (including any accompanying hydrogens by valence e.g. (non-limiting) OH, NH₂, SH, SiH₃, PH₂ etc.), for example hydrogen, or deuterium or fluorine;
C^{f}, C^{g}, C^{h} and C^{w} are each independently selected from a single bond, O, S, Se, NR^{V}, PR^{V}, BR^{V}, C(R^{V})₂ or Si(R^{V})₂, wherein each RY is independently selected from a constituent group of L (defined earlier);
x, w, f, g, h are independently selected to be 0, 1, 2 or 3;
d is a selected integer between 0 and 7;
k, s and s^{k} are independently selected to be 0, 1, 2, or 3;

The 5-sided ring structure is attached by any one of its available ring atoms, and none, one or two of its bonds can be a double bond, for example at locations shown by the “single or double bond” symbol; R₁ and R₅ are attached to any available carbon atom of phenyl rings A^{a} and B^{b}, respectively, and at each occurrence are independently selected from PH₂, OH, SH, hydrogen, deuterium, alkyl, substituted alkyl, trifluoromethoxy, halogen, haloalkyl, cyano, nitro, OR⁸, NR⁸R⁹, C(═O)R₈, CO₂R₈, C(═O)NR₈R₉, NR₈C(═O)R₉, NR₈C(═O)OR₉,
S(O)ₒR₉, S(O)R₉, SO₂R₉, S(O)₂R₉, SR₉, NR₈SO₂R₉, SO₂NR₈R₉, cycloalkyl, heterocyclo, aryl, and heteroaryl, and/or two of R1 and/or two of R5 join together to form a fused benzo ring;
R₂, R₃ and R₄ are independently selected from E (defined earlier), hydrogen, or deuterium, or alkyl, or deuterated alkyl, and substituted alkyl, or one of R₂, R₃ and R₄ is a bond to R, T or Y and the other of R₂, R₃ and R₄ are independently selected from hydrogen, alkyl, and substituted alkyl;
Z and Y are independently selected from C(═O), —CO₂—, —SO₂—, —CH₂—, —CH₂C(═O)—, and —C(═O) C(═O)—, or Z may be absent;
R and T are selected from —CH₂—, —C(═O)—, and —CH[(CH₂)ₚ(Q)]-, wherein Q is NR₁₀R₁₁, OR₁₀ or CN;
R₆ is selected from thienyl, alkyl, alkenyl, substituted alkyl, substituted alkenyl, aryl, substituted aryl, cycloalkyl, heterocyclo, C₂₋₄alkenyl, heteroaryl and aryl optionally substituted with a lower aliphatic group or one or more functional groups selected independently from the group consisting of —NH₂, —OH, phenyl, halogen, (C₁-C₄)alkoxy or —NHCOCH₃;
R₇ is selected from L (defined earlier), PH₂, OH, SH, hydrogen, alkyl, substituted alkyl, alkenyl, substituted alkenyl, aminoalkyl, halogen, haloalkyl, cyano, nitro, keto (═O), hydroxy, alkoxy, alkylthio, C(═O)H, acyl, CO₂H, alkoxycarbonyl, carbamyl, sulfonyl, sulfonamidyl, cycloalkyl, heterocyclo, aryl, and heteroaryl;
R₈ and R₉ are independently selected from hydrogen, alkyl, substituted alkyl, C₂₋₄ alkenyl optionally substituted, cycloalkyl, heterocyclo, aryl, and heteroaryl, or R₈ and R₉ taken together to form a heterocyclo or heteroaryl;
R₁₀ and R₁₁ are independently selected from hydrogen, alkyl, and substituted alkyl;
m and n are independently selected from 0, 1, 2 and 3
o, p and q are independently 0, 1 or 2; and
r and t are 0 or 1.

### Preferred Compounds of Formula (II)

Preferred methods are to use, and preferred compounds are, compounds with the following formula, or pharmaceutically-acceptable salts, solvates, hydrates or prodrugs thereof,

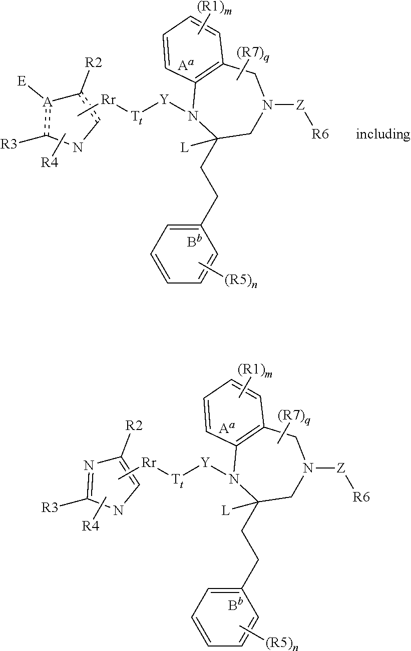

wherein:
L is hydrogen, or deuterium, or methyl, or hydroxyalkyl, or fluorine;
A is nitrogen (N), or N⁺, or carbon;
E is absent, or alkyl, or substituted alkyl, or deuterated alkyl, or aminoalkyl, or thioalkyl, or alkoxy or any atom or isotope permitted by valence (including any accompanying hydrogens by valence e.g. (non-limiting) OH, NH₂, SH, SiH₃, PH₂ etc.), for example hydrogen or deuterium;
R₁ and R₅ are attached to any available carbon atom of phenyl ring A^{a} and phenyl ring B^{b}, respectively, and at each occurrence are independently selected from hydrogen, deuterium, alkyl, aralkyl, aminoalkyl, halogen, cyano, nitro, hydroxy, alkoxy, trifluoromethoxy, alkylthio, NH₂, NH(alkyl), N(alkyl)₂, C(═O)H, acyl, CO₂H, alkoxycarbonyl, carbamyl, sulfonyl, sulfonamide, cycloalkyl, heterocyclo, aryl, and heteroaryl, and/or two of R₁ and/or two of R₅ join together to form a fused benzo ring;
R₂, R₃ and R₄ are independently selected from hydrogen and alkyl;
Z is —CO₂—, —SO₂—, or is absent;
Y, R and T are selected from —CH₂— and —C(═O)—,
R₆ is selected from:
C₁₋₄alkyl or C₁₋₄alkenyl optionally substituted with up to three (selected independently) of halogen, aryl and CO₂C₁₋₆alkyl;
phenyl optionally substituted with up to three (selected independently) R₁₂ and/or having fused thereto a benzo-ring or a five to six membered heteroaryl;
heteroaryl selected from thiophenyl, imidazolyl, pyrazolyl, and isoxazolyl wherein said heteroaryl is optionally substituted with up to two (selected independently) R₁₂,
R₇ is selected from hydrogen, alkyl, substituted alkyl, alkenyl, substituted alkenyl, aminoalkyl, halogen, cyano, nitro, keto (═O), hydroxy, alkoxy, alkylthio, C(═O)H, acyl, CO₂H, alkoxycarbonyl, carbamyl, sulfonyl, sulfonamide, cycloalkyl, heterocyclo, aryl, and heteroaryl;
R₁₂ at each occurrence is independently selected from each other R₁₂ from the group consisting of C₁₋₆alkyl, halogen, nitro, cyano, hydroxy, alkoxy, NHC(═O)alkyl, —CO₂alkyl, —SO2phenyl, aryl, five to six membered monocyclic heteroaryl, and phenyloxy or benzyloxy in turn optionally substituted (selected independently) with halogen, hydroxyl, C₁₋₄alkyl, O(C₁₋₄alkyl);
m and n are independently selected from 0, 1, 2 or 3; and
q is 0, 1 or 2; and
r and t are 0 or 1.

More preferred are compounds having the following formula, or pharmaceutically-acceptable salts, solvates, hydrates or prodrugs thereof,

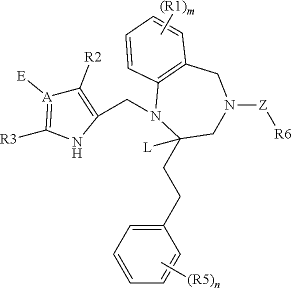

wherein
R₁ and R₅ are attached to any available carbon atom of phenyl ring A^{a} and phenyl ring B^{b}, respectively, and at each occurrence are independently selected from alkyl,
halogen, cyano, hydroxy, alkoxy, NH₂, NH (alkyl), N (alkyl)₂, C(═O)H, acyl, CO₂H, alkoxycarbonyl, and/or two of R₁ and/or two of R₅ join together to form a fused benzo ring;
R₂, R₃ and R₄ are independently selected from hydrogen and lower alkyl;
Z is —CO₂—, —SO₂—, or is absent;
R₆ is selected from:
C₁₋₄alkyl or C₁₋₄alkenyl optionally substituted with up to three (selected independently) of halogen, aryl and CO₂C₁₋₆alkyl;
phenyl optionally substituted with up to three (selected independently) R₁₂ and/or having fused thereto a benzo ring or a five to six membered heteroaryl;
heteroaryl selected from thiophenyl, imidazolyl, pyrazolyl, and isoxazolyl, wherein said heteroaryl is optionally substituted with up to two (selected independently) R₁₂,
R₁₂ at each occurrence is independently selected from each other R₁₂ from the group consisting of C₁₋₆ alkyl, halogen, nitro, cyano, hydroxy, alkoxy, NHC(═O)alkyl,
—CO₂alkyl, —SO₂phenyl, aryl, five to six membered monocyclic heteroaryl, and phenyloxy or benzyloxy in turn optionally substituted with (selected independently) halogen, hydroxyl, C₁₋₄ alkyl, and/or O(C₁₋₄ alkyl); and
m and n are independently selected from 0, 1, or 2.

Even more preferred are compounds as immediately defined above wherein R⁶ is selected from C₁₋₄alkyl, trifluoromethyl, benzyl, C₂₋₃alkenyl substituted with phenyl,

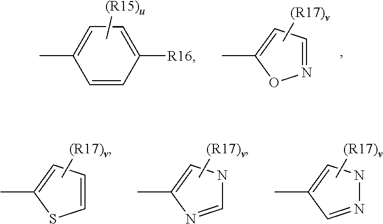

wherein:
R₁₅ is halogen, alkyl, nitro, cyano, hydroxy, alkoxy, NHC(═O)alkyl, and/or two R₁₅ groups are taken together to form a fused benzo ring or a five to six membered heteroaryl;
R₁₆ is selected from hydrogen, deuterium, halogen, alkyl, nitro, cyano, hydroxy, alkoxy, NHC(═O)alkyl, and phenyloxy or benzyloxy in turn optionally substituted with 1 to 3 groups independently selected from hydrogen, deuterium, halogen, cyano, C₁₋₄alkoxy;
R₁₇ is selected from alkyl, alkoxy, CO₂C₁₋₆alkyl, and SO₂phenyl;
and u and v are independently 0, 1 or 2.

Most preferred compounds of Formula (II) are those having the formula:

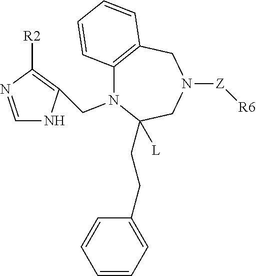

wherein
L is deuterium;
R₂ is hydrogen or CH₃;
Z is —CO₂—, —SO₂—, or is absent; and
R₆ is selected from the groups recited immediately above, most preferably

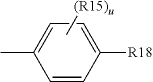

### Some Example Embodiments of Formula (II)

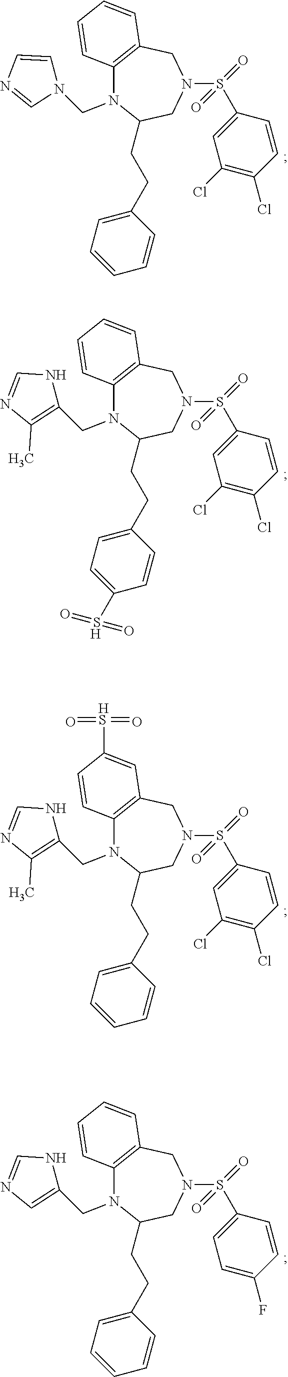

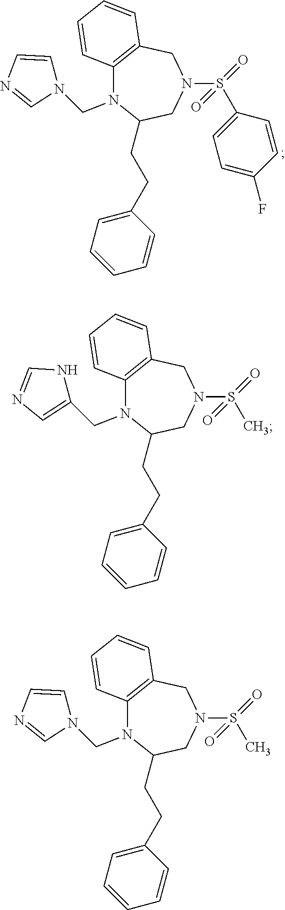

Scheme IIa is a route for synthesizing Compound 31 [10], starting reagents are commercially available from multiple suppliers listing on the LabNetwork (www.labnetwork.com) e.g. Compounds 1, 7 and 9 are available from Astatech Inc., Bristol PA, USA, Compound 2 from Stru Chem, Wujiang city, China.

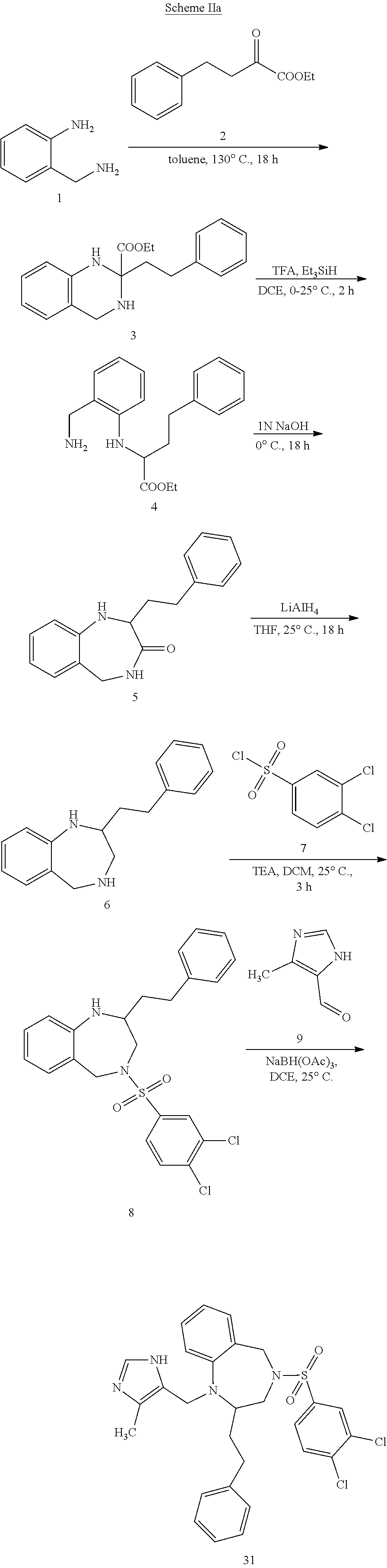

Product of Scheme IIa, Compound 31: LC retention time (RT)=0.87 minutes, MS (electrospray ionisation, positive mode): m/z 554.90 [M+H]⁺, 576.90 [M+Na]⁺, 278.90 [M+2H]²⁺, (all Observed m/z are within 0.3 Daltons of Expected: 555.14 [M+H]⁺, 577.12 [M+Na]⁺, 278.07 [M+2H]²⁺); ¹H NMR (400 MHZ, Methanol-d4) δ (ppm) 8.35 (s, 1H), 8.14-8.06 (m, 1H), 8.04 (d, J=2.1 Hz, 1H), 7.84-7.71 (m, 2H), 7.27 (dt, J=22.4, 7.4 Hz, 4H), 7.16 (dd, J=8.0, 5.6 Hz, 2H), 7.09 (d, J=7.5 Hz, 2H), 7.03 (t, J=7.4 Hz, 1H), 4.71 (d, J=13.9 Hz, 1H), 4.37 (d, J=14.2 Hz, 1H), 4.29 (d, J=14.2 Hz, 1H), 3.96 (dd, J=19.7, 13.4 Hz, 2H), 3.17-3.10 (m, 1H), 2.78 (d, J=12.8 Hz, 1H), 2.69 (ddd, J=14.5, 9.7, 5.3 Hz, 1H), 2.54 (dt, J=13.7, 8.6 Hz, 1H), 2.11 (s, 3H), 1.50 (s, 1H), 1.36 (dtd, J=14.1, 9.1, 5.1 Hz, 1H) {NMR probe temperature=297.9 K}.

¹H NMR (400 MHZ, Chloroform-d) δ (ppm) 8.21 (s, 1H), 7.93 (d, J=2.1 Hz, 1H), 7.82 (s, 1H), 7.65 (dd, J=8.4, 2.1 Hz, 1H), 7.58 (d, J=8.4 Hz, 1H), 7.30 (s, OH), 7.26 (s, 2H), 7.20 (t, J=7.3 Hz, 1H), 7.10 (d, J=7.4 Hz, 2H), 7.07-6.98 (m, 2H), 4.60 (d, J=13.8 Hz, 1H), 4.32 (d, J=14.1 Hz, 1H), 4.12 (dd, J=24.1, 14.0 Hz, 2H), 3.82 (d, J=12.8 Hz, 1H), 3.08 (s, 2H), 2.91 (d, J=13.0 Hz, 1H), 2.76-2.64 (m, 1H), 2.49 (dt, J=14.8, 8.2 Hz, 1H), 2.11 (s, 3H), 1.58-1.43 (m, 1H) {NMR probe temperature=298.5 K}.

¹³C NMR (101 MHZ, Methanol-d4) δ (ppm) 149.14, 142.91, 140.73, 138.20, 134.62, 134.51, 132.72, 132.22, 130.62, 130.05, 129.75, 129.50, 129.39, 129.20, 128.48, 127.87, 127.01, 123.78, 121.75, 58.88, 53.98, 53.69, 48.11, 33.55, 31.58, 9.53 {NMR probe temperature=298.0 K}.

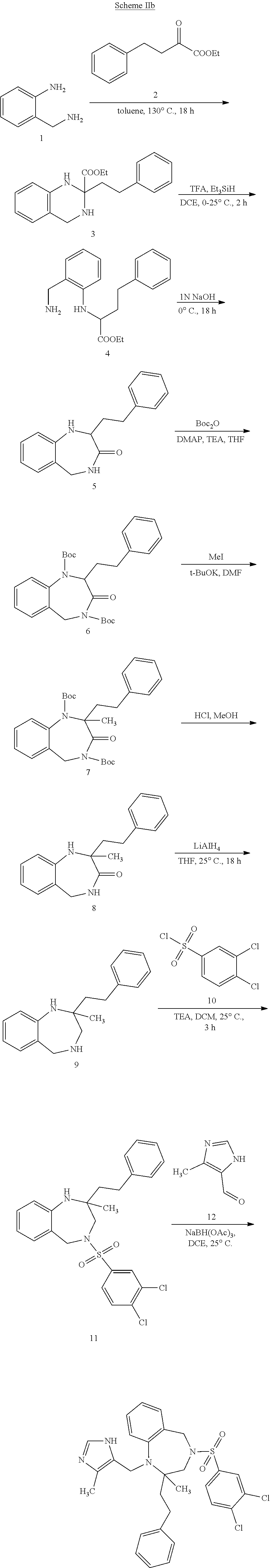

The reaction schemes below, Scheme IIc, Scheme IId, Scheme IIe and Scheme IIf, show only starting material(s) and product because they use the same internal steps as Scheme IIa (not shown), but with a different starting material, a different Compound 1 in the case of Scheme IIc, Scheme IId, Scheme IIe, or a different Compound 2 in the case of Scheme IIf, and thence different products are produced as shown. Starting compounds are available from suppliers listed on www.labnetwork.com: e.g. Compound 1 of Scheme IIc and Scheme IIe from Fluorochem, Hadfield, Derbyshire, UK, e.g. Compound 1 of Scheme IId from J&W Pharmlab LLC, Levittown, PA, USA, e.g. Compound 2 of Scheme IIf from Arena Chemical, La Mure, France, Compound 1 of Scheme IIf from Astatech Inc., Bristol PA, USA.

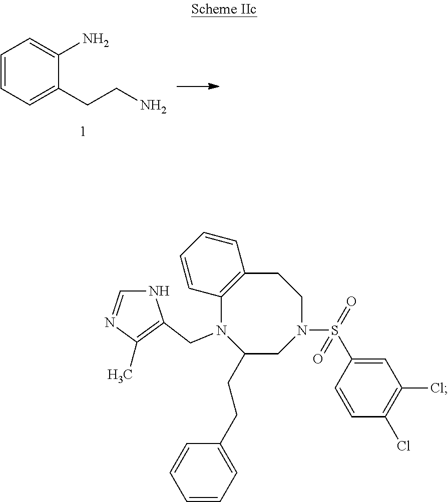 [SMILES: NCCc1ccccc1N ; Cc1nc[nH]c1CN1c2ccccc2CCN(S(=O)(=O)c2ccc(Cl)c(Cl)c2)CC1CCc1ccccc1]

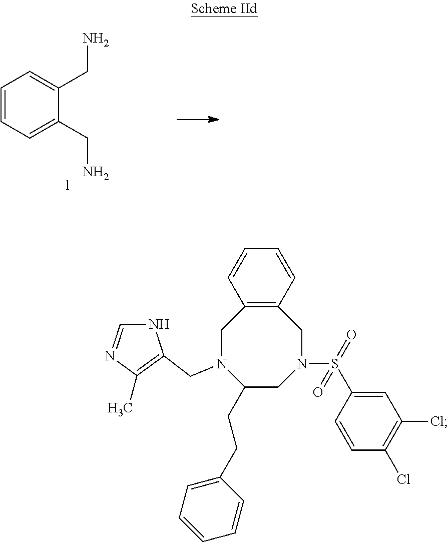 [SMILES: NCc1ccccc1CN ; Cc1nc[nH]c1CN1Cc2ccccc2CN(S(=O)(=O)c2ccc(Cl)c(Cl)c2)CC1CCc1ccccc1]

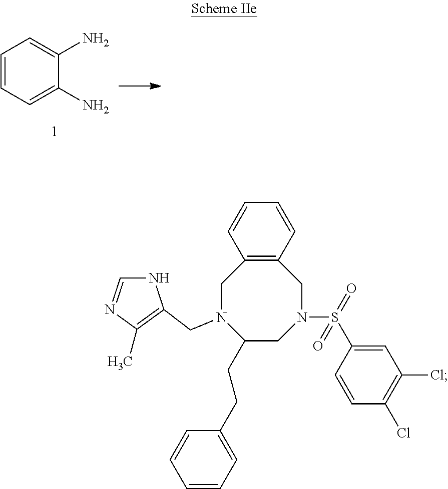 [SMILES: Nc1ccccc1N ; Cc1nc[nH]c1CN1Cc2ccccc2CN(S(=O)(=O)c2ccc(Cl)c(Cl)c2)CC1CCc1ccccc1]

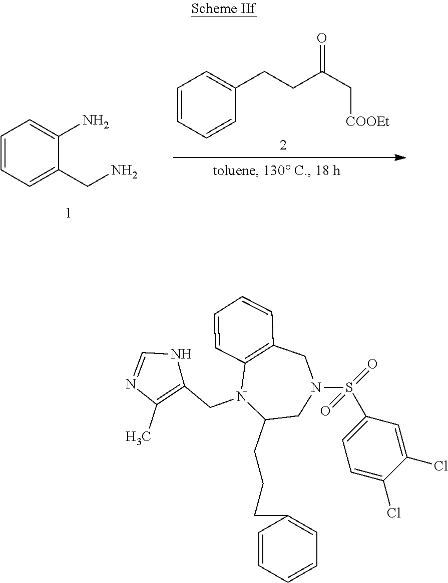 [SMILES: NCc1ccccc1N ; CCOC(=O)CC(=O)CCc1ccccc1 ; Cc1nc[nH]c1CN1c2ccccc2CN(S(=O)(=O)c2ccc(Cl)c(Cl)c2)CC1CCCc1ccccc1]

The reaction schemes below, Scheme IIg and Scheme IIh are the same as Scheme IIa up until Compound 6. This shared component isn't shown, just the point of divergence from Compound 6, which comes from using a different Compound 7, wherein the Compound 7 options shown in Scheme IIg and Scheme IIh are available from multiple suppliers on www.labnetwork.com (e.g. available from Fluorochem, Hadfield, Derbyshire, UK).

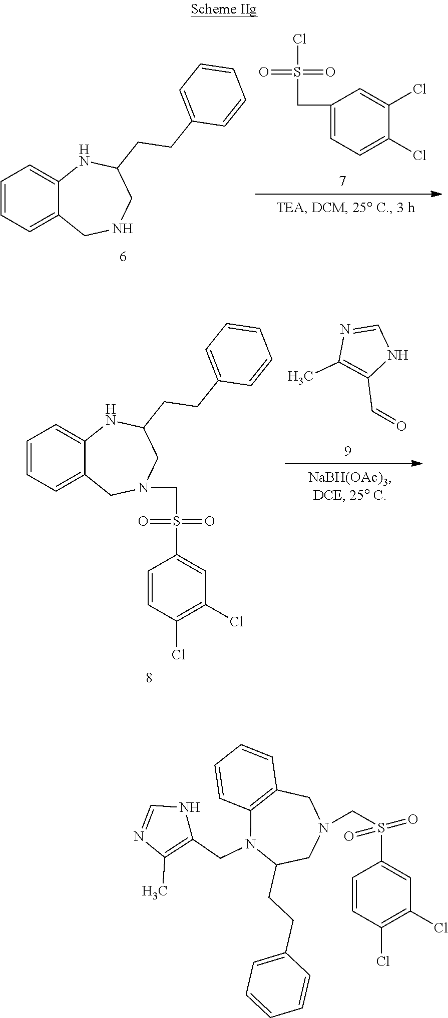

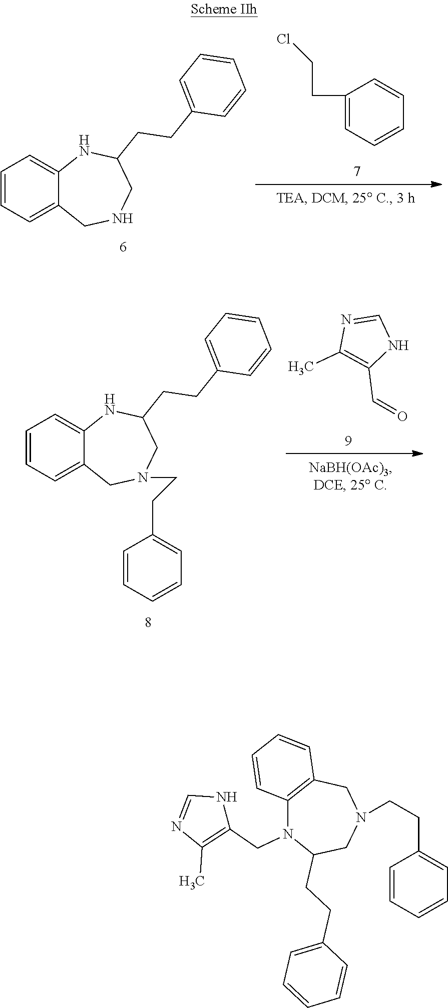

The reaction schemes below, Scheme IIi, Scheme IIj, Scheme IIk and Scheme IIl are the same as Scheme IIa up until Compound 8. This shared component isn't shown, just the point of divergence from Compound 8, which comes from using a different Compound 9, wherein the Compound 9 options shown in Scheme IIi, Scheme IIj, Scheme IIk and Scheme IIl are all available from multiple suppliers on www.labnetwork.com (e.g. all, except that for Scheme IIl, available from Fluorochem, Hadfield, Derbyshire, UK, Compound 9 from Scheme IIl available from Matrix Scientific, Columbia, SC, USA).

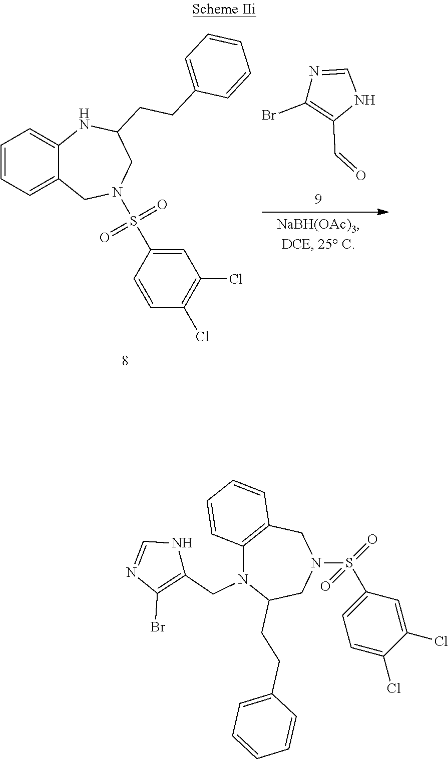 [SMILES: O=S(=O)(c1ccc(Cl)c(Cl)c1)N1Cc2ccccc2NC(CCc2ccccc2)C1 ; O=Cc1[nH]cnc1Br ; O=S(=O)(c1ccc(Cl)c(Cl)c1)N1Cc2ccccc2N(Cc2[nH]cnc2Br)C(CCc2ccccc2)C1]

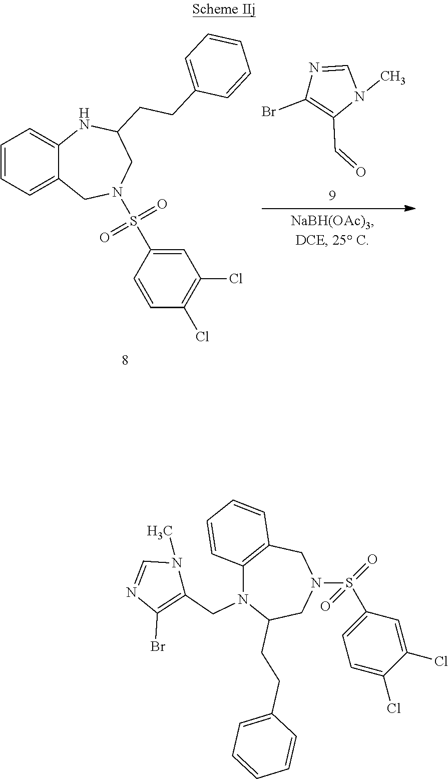

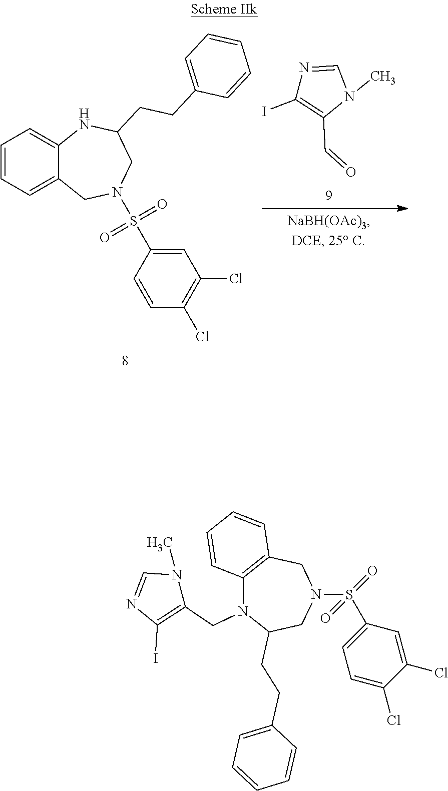

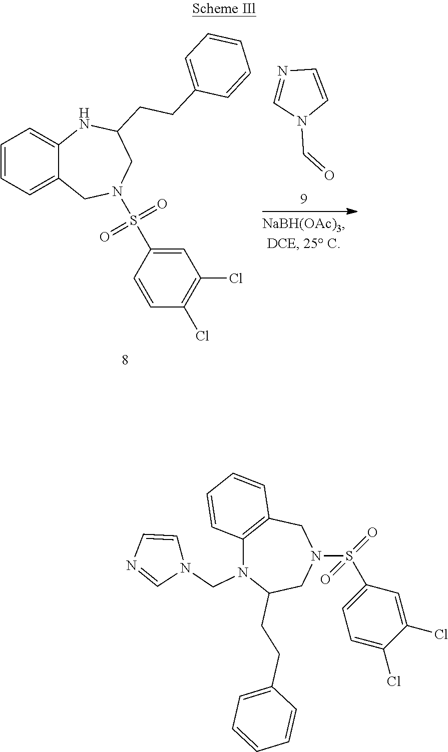

### Example (III)

### Summary of Formula (III)

This disclosure embodiment relates to compounds having the following formula: Formula (III)

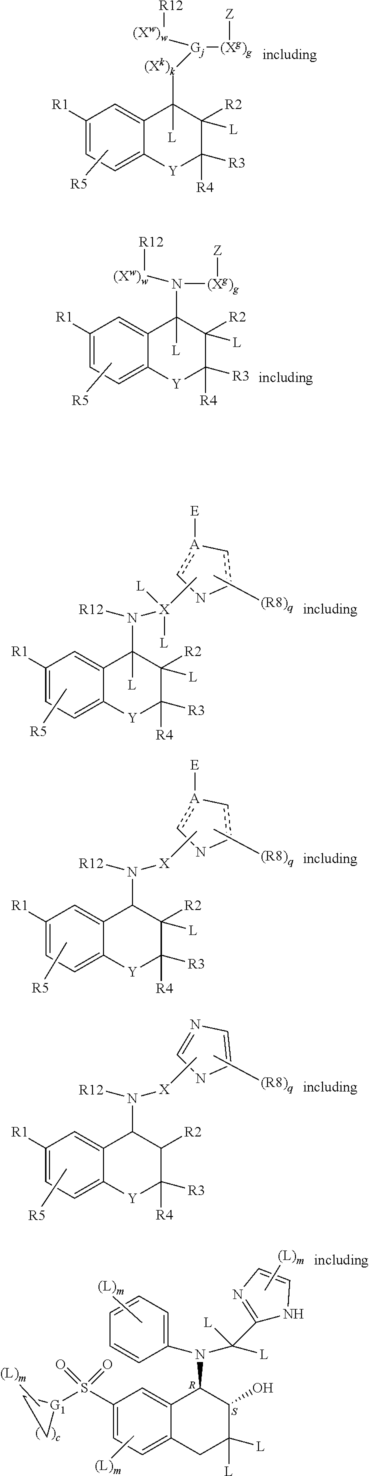

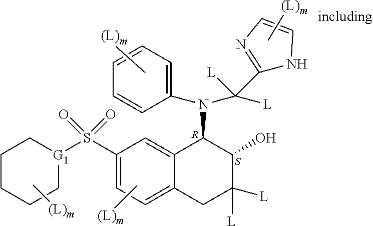

or their enantiomers, diastereomers, pharmaceutically-acceptable salts, solvates, hydrates or prodrugs thereof, wherein:

Optionally, one or more places have deuterium in place of hydrogen, at an artificially high level of deuterium incorporation, in excess of the naturally occurring abundance;

Optionally, one or more places have fluorine, or other halogen, or methyl, or alkyl, or substituted alkyl, in place of hydrogen;
Z is heteroaryl;
g, w and k are independently selected from 0, 1, 2, 3, 4;
L is independently at each point of its use hydrogen, alkyl, or substituted alkyl (non-limiting example: CF₃), or deuterated alkyl (non-limiting example: CD₃), or aminoalkyl, or thioalkyl, or alkoxy, or halogen, or haloalkyl, or haloalkoxy, or hydroxyalkyl, or any atom or isotope permitted by valence (including any accompanying hydrogens by valence e.g. (non-limiting) OH, NH₂, SH, SiH₃, PH₂ etc.); X^{g}, X^{w} and X^{k} are independently selected from a single bond, O, S, Se, NR^{V}, PR^{V}, BR^{V}, C(R^{V})₂ or Si(R^{V})₂, wherein each RY is independently selected from a constituent group of L (defined earlier);
G₁ is, independently at each point of use, N or CH;
c is selected from 1, 2, 3, 4, 5, 6, 7, 8, 9;
m is independently at each point of use selected from 0, 1, 2, 3, 4, 5, 6, as valence permits;
R2 is hydrogen, L (defined earlier), hydroxyl (—OH), SH, NH₂, methyl, alkoxy, substituted alkoxy, haloalkoxy, ether, halogen or —OC (O)R14;
R14 is hydrogen, alkyl, haloalkyl, aryl, arylalkyl, cycloalkyl or (cycloalkyl)alkyl;
R3 and R4 are each independently hydrogen, or L (defined earlier), or CF₃, or NH₂, or OH, or chlorine or other halogen, or alkyl, or substituted alkyl, or deuterated alkyl, or arylalkyl, or R3 and R4 taken together with the carbon atom to which they are attached form a 3- to 7-membered carbocyclic ring;
R5 is independently at each point of use hydrogen, L (defined earlier), PH2, OH, SH, alkyl, substituted alkyl, halogen, nitrile, haloalkyl, aryl, heteroaryl, cycloalkyl or heterocyclo;
R12 is selected from hydrogen, deuterium, alkyl, aryl, heteroaryl, cycloalkyl, heterocyclo;
X is alkyl;
Y is a single bond, —CH₂—, —C(O)—, —O—, —S—, —N(R14)- or (X^{f})_{f} where X^{f} is selected from a single bond, O, S, NR^{V} or C(R^{V})₂, wherein each R^{V} is independently selected from a constituent group of L (defined earlier);
f is 0, 1, 2 or 3;
A is nitrogen (N), or N⁺, or carbon;
E is absent, or alkyl, or substituted alkyl (non-limiting example: CF₃), or deuterated alkyl, or aminoalkyl, or thioalkyl, or alkoxy or any atom or isotope permitted by valence (including any accompanying hydrogens by valence e.g. (non-limiting) OH, NH₂, SH, SiH₃, PH₂ etc.), for example hydrogen, or deuterium, or fluorine;

R8 is independently selected at each point of use from E (defined earlier), hydrogen, alkyl, halogen, carbamyl, carbamylC₁₋₄alkyl, substituted alkyl or two R8 groups join to form an optionally substituted fused phenyl ring;
q is 0, 1, 2, 3 or 4.
R1 is selected from L (defined earlier), hydrogen, deuterium, CN, SO₂-piperidine, SO₂-piperidine substituted with 0-10 (selected independently) of R5, R9, cyano, halogen, alkyl, substituted alkyl, alkenyl, substituted alkenyl, alkylene, substituted alkylene, alkynyl, substituted alkynyl, alkoxy, thioalkyl, aminoalkyl, carbamyl, sulfonyl, sulfonamide, cycloalkyl, (cycloalkyl)alkyl, hydroxyalkyl, haloalkyl, haloalkoxy, alkoxyalkyl, morpholinylalkyl, aryl, arylalkyl, heterocyclo, heteroaryl, (heterocyclo)alkyl, (heteroaryl)alkyl, acyl, alkoxycarbonyl, substituted amino;

Most preferably R1 is smaller than 300 Daltons;
R9 is

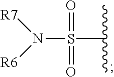

R6 and R⁷ are independently hydrogen, L (defined earlier), R1 (provided R1 is not R9), alkyl, aryl, heteroaryl, cycloalkyl, heterocyclo, arylalkyl, (heteroaryl)alkyl, haloalkyl, hydroxyalkyl, hydroxyalkyl substituted with a carboxylic ester and/or carboxylic acid, alkoxyalkyl, thioalkyl, (cycloalkyl)alkyl, morpholinylalkyl, heterocyclo or (heterocyclo)alkyl; or R6 and R7 taken together with the nitrogen atom to which they are attached form a 5- to 7-membered mono or bicyclic ring including fused rings such as 1-pyrrolidinyl, 1-piperidinyl, 1-azepinyl, 4-morpholinyl, 4-thiamorpholinyl, 4-thiamorpholine dioxide, 1-piperazinyl, 4-alkyl-1-piperazinyl, 4-arylalkyl-1-piperazinyl, 4-diarylalkyl-1-piperazinyl; or 1-piperazinyl, l-pyrrolidinyl, 1-piperidinyl or 1-azepinyl substituted with one or more independently selected L (defined earlier), alkyl, alkoxy, alkylthio, halo, trifluoromethyl, hydroxy, aryl, arylalkyl, —COOR14 or —CO-substituted amino;
or R5 and R6 taken together with the atoms to which they are attached form a 5- to 7-membered ring optionally substituted with aryl;
Encompassed by this disclosure are methods of administering a therapeutically effective amount of any compound(s) of [P6], or a pharmaceutically-acceptable salt, solvate, hydrate or prodrug thereof, optionally in a pharmaceutical composition(s), optionally in co-therapy with another anti-cancer treatment(s), to treat/ameliorate/prevent/combat cancer in a subject. Especially preferred for this use are compounds of [P6] with 3S, 4R stereochemistry.

### Preferred Compounds of Formula (III)

Preferred methods are to use, and preferred compounds are, compounds of Formula (III), their enantiomers, diastereomers, pharmaceutically-acceptable salts, solvates, hydrates or prodrugs thereof, in which:
Z is triazolyl optionally substituted with one to two (selected independently) R8 or imidazolyl optionally substituted with one to two (selected independently) R8 and/or having fused thereto a benzene ring in turn optionally substituted with one to two R8 (selected independently);
Y is oxygen;
R2 is hydroxyl;
R3 and R4 are methyl or chlorine;
R1 is R9;
G₁ is nitrogen;
R6 and R⁷ are alkyl; or R6 and R⁷ taken together with the nitrogen atom to which they are attached (G₁=N) form a 6-membered ring;

X is alkyl;
R12 is aryl or heterocyclo;
A is N;
E is absent, or deuterium, or hydrogen;
R5 and R⁸ are hydrogen;
Stereochemistry is 3S, 4R;

### Some Example Embodiments of Formula (III)

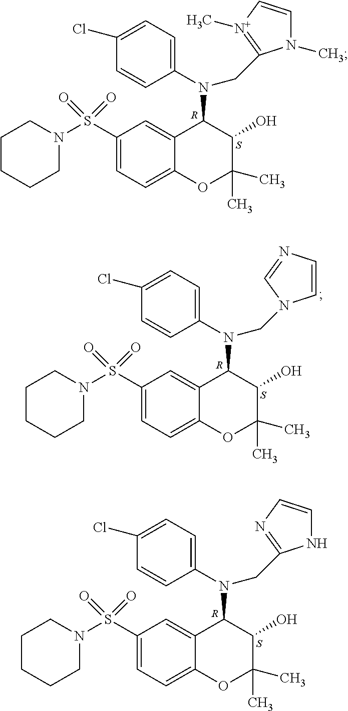

### Example (IV)

Background: Well known to those of the art: amino acids have the following structure, wherein the R group is different in different amino acids.

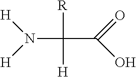

### Summary of Formula (IV)

This disclosure embodiment relates to compounds having the following formula: Formula (IV)

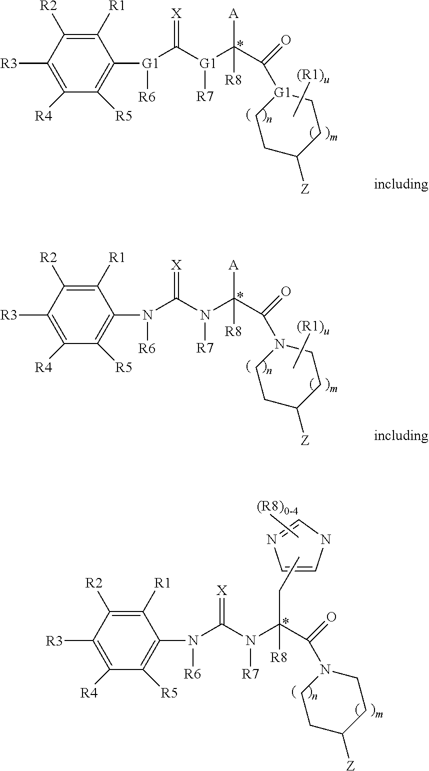

or their enantiomers, diastereomers, pharmaceutically-acceptable salts, solvates, hydrates or prodrugs thereof, wherein:
G1 is, independently at each point of use, N or CH;
u is selected from 0, 1, 2, 3, 4, 5, 6, 7, 8;
X is selected from O or S;
A is selected from hydrogen, deuterium, alkyl, substituted alkyl, cycloalkyl, heterocyclo, aryl, heteroaryl, aminoalkyl, thioalkyl, alkoxy and an R group of a proteogenic amino acid, or other amino acid synthesized or used by a living system (non-limiting example of such a system: a human), which is optionally isotopically enriched, and/or substituted by alkyl, substituted alkyl, deuterated alkyl, halogen, cycloalkyl, heterocyclo, aryl, heteroaryl, aminoalkyl, thioalkyl, alkoxy, haloalkyl, haloalkoxy, or any atom or isotope permitted by valence (including any accompanying hydrogens by valence e.g. (non-limiting) OH, NH₂, SH, SiH₃, PH₂ etc.);
n and m are 0, 1, or 2;
R₁ through R₅ are independently selected from hydrogen, halogen, NO₂, PH₂, OH, SH, CN, C₁₋₈alkyl, substituted C₁₋₈alkyl, C₃₋₈cycloalkyl, aryl, heterocyclo, heteroaryl, OR₉, SR₉, COR₁₁, CO₂R₁₁, CONR₉R₁₀ or NR₉R₁₀;
R₆ and R⁷ are independently hydrogen, alkyl or substituted alkyl;
R₈ is hydrogen, deuterium, C₁₋₈alkyl, substituted C₁₋₈alkyl, deuterated C₁₋₈alkyl, aryl, heterocyclo, heteroaryl, aminoalkyl, thioalkyl, alkoxy, halogen, haloalkyl, haloalkoxy, or any atom or isotope permitted by valence (including any accompanying hydrogens by valence e.g. (non-limiting) OH, NH₂, SH, SiH₃, PH₂ etc.);
Z is hydrogen, alkyl, substituted alkyl, cycloalkyl, aryl, heterocyclo, heteroaryl, COR₁₁, CO₂R₁₁, SO₂R₁₁, S(O)R₁₁ or CONR₉R₁₀;
R₉ and R₁₀ are independently hydrogen, C₁₋₈alkyl, substituted C₁₋₈alkyl, C₃₋₁₀cycloalkyl, aryl, heterocyclo, heteroaryl, COR₁₃, SO₂R₁₃ or S (O)R₁₃; and
R₁₁, R₁₂ and R13 are independently hydrogen, C₁₋₈alkyl, substituted C₁₋₈alkyl, C₃₋₁₀cycloalkyl, aryl, heterocyclo or heteroaryl;
wherein each occurrence of R₉-R₁₃ is chosen independently.

Preferred compounds of Formula (IV)

Preferred methods are to use, and preferred compounds are, compounds of Formula (IV), their enantiomers, diastereomers, pharmaceutically-acceptable salts, solvates, hydrates or prodrugs thereof, in which:
R₂, R₃ and R4 are all hydrogen; and/or
R₆ and R₇ are both hydrogen; and/or
n and m are both 1; and/or
R₁ and R₅ are both C₁₋₈ alkyl, preferably both R₁ and R₅ are isopropyl groups.

Other preferred methods use, and preferred compounds are, compounds of Formula (IV), their enantiomers, diastereomers, pharmaceutically-acceptable salts, solvates, hydrates or prodrugs thereof, in which:
Z is C₁₋₈alkyl, C₂₋₈alkenyl, C₁₋₈haloalkyl, cycloalkyl, aryl, arylalkyl, heteroaryl, heteroarylalkyl-COR₁₁, —CO₂R₁₁, —SO₂R₁₁, —S(O)R₁₁ or —CONR₉R₁₀; especially preferable is benzyl, —C(O)₂H or —C(O)₂C₁₋₈alkyl;
R₉ is hydrogen;
R₁₀ is C₁₋₈alkyl or C₃₋₁₀cycloalkyl; aryl or arylalkyl; and
R₁₁ is hydrogen, C₁₋₈alkyl, C₃₋₁₀cycloalkyl, C₃₋₁₀heterocycloalkyl, C₃₋₁₀aryl or C₃₋₁₀ arylalkyl.

Other preferred methods use, and preferred compounds are, compounds of Formula (IV), their enantiomers, diastereomers, pharmaceutically-acceptable salts, solvates, hydrates or prodrugs thereof, in which:
A is hydrogen, deuterium, C₁₋₈alkyl, aminoalkyl, heteroaryl, aryl, or alkyl substituted with one or more substituents independently selected from heterocyclo, aryl, OH, SH, ST¹, —C(O), H, T³-NT⁵T⁶, -T⁸-C(O)ₜT⁹-NT⁵T⁶ or T³-N(T²) T⁴NT⁵T⁶,
T¹ is alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl;
T² and T³ are each independently a single bond, -T⁸-S(O)ₜ-T⁹-, -T⁸-C(O)-T⁹-, -T¹⁸-C(S)-T⁹, -T⁸-S-T⁹-, -T⁸-O—C(O)-T⁹-, -T⁸-C(O)ₜT⁹-, -T⁸-C(═NT¹⁰)-T⁹- or -T⁸-C(O)—C(O)-T⁹-;
T⁵, T⁶, T⁷, T⁸ and T⁹ are independently hydrogen, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alky, each group optionally substituted where valence allows by one to three groups independently selected from halo, cyano, nitro, OH, oxo, —SH, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl, —OT¹¹, —ST¹¹, —C(O)ₜH, —C(O)ₜT¹¹, —O—C(O)T¹¹, TC(O)ₜN(T¹²)T¹¹, —SO₃H, —S(O)ₜT¹¹, S(O)ₜN(T¹²)T¹¹, -T¹³-NT¹¹T¹², -T¹³-N(T¹²)-T⁴-NT¹¹T²², -T¹³-N(T¹¹)-T¹²-T¹¹ and -T¹³-N(T¹⁸)-T¹⁴-H; or
T8 and T⁹ are each independently a single bond, alkylene, alkenylene or alkynylene;
T¹¹ is alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl;
T¹² is halo, cyano, nitro, OH, oxo, —SH, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl, —C(O)ₜH or —SO₃H;
T¹³ and T¹⁴ are each independently a single bond, —S(O)—, —C(O)—, —C(S)—, —O—, —S—, —O—C(O)—, —C(O)—, —C(═NT¹³)— or —C(O)—C(O)—;
wherein each occurrence of T1-T14 is chosen independently; and
t is 1 or 2.

Preferred compounds of the foregoing section are those in which A is hydrogen, deuterium, C₁₋₈alkyl, aminoalkyl, hydroxyalkyl, heterocycloalkyl, heteroaryl alkyl, aryl, arylalkyl, or alkyl substituted with a group selected from SH, ST⁴, —C(O)ₜH, T⁶-NT⁸T⁹, -T¹¹-C(O)ₜT¹²-NT⁸T⁹ and T⁶-N(T⁵)T⁷NT⁸T⁹. More preferred are those compounds in which A is hydrogen, deuterium, methyl, —CH₂ (CH₃)₂, —(CH₂)₂(CH₃)₂, —CH(CH₃)CH₂(CH₃), —(CH₂)OH, hydroxyethyl, —(CH₂)₂SCH₃, —CH₂SH, phenyl, —CH₂(phenyl), —CH₂(p-hydroxyphenyl), —CH₂(indole), —(CH₂)C(O)NH₂, —(CH₂)₂C(O)NH₂, —(CH₂)₂C(O)OH, —CH₂C(O)OH, —(CH₂)₄NH₂, —(CH₂)₃(═NH)CNH₂, or —CH₂(imidazole). Especially preferred A groups are —CH(CH₃)CH₂(CH₃), phenyl, phenyl alkyl or —CH₂(2-indole).

Alternatively preferred methods use, and preferred compounds are, compounds of Formula (IVb), their enantiomers, diastereomers, pharmaceutically-acceptable salts, solvates, hydrates or prodrugs thereof, in which: Formula (IVb)

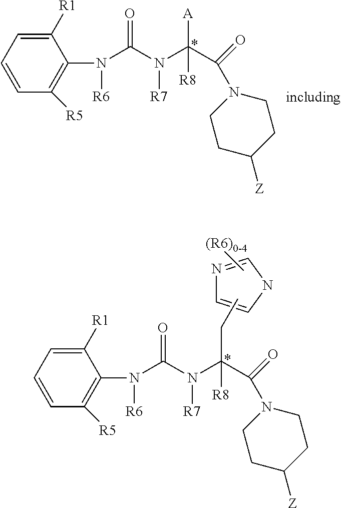

wherein:
A is selected from hydrogen, deuterium, C₁₋₈alkyl, aminoalkyl, substituted alkyl, deuterated alkyl, aryl, heteroaryl, or alkyl substituted with one or more substituents independently selected from heterocyclo, aryl, heteroaryl, OH, SH, ST¹, —C(O)ₜH, T³-NT⁵T⁶, -T⁸-C(O)ₜT⁹-NT⁵T⁶ or T³-N(T²) TANT⁵T⁶;
R¹ and R⁵ are independently C₁₋₈alkyl optionally substituted where valence allows;
R⁶ and R⁷ are independently hydrogen or C₁₋₈alkyl;
R₈ is hydrogen, halogen, deuterium, C₁₋₈alkyl or substituted C₁₋₈alkyl;
Z is hydrogen, C₁₋₈alkyl, C₂₋₈alkenyl, C₁₋₈haloalkyl, cycloalkyl, aryl, arylalkyl, heteroaryl, heteroarylalkyl —COR¹¹, —CO₂R¹¹, —SO₂R¹¹, —S(O)R¹¹ or —CONR⁹R¹⁰;
R⁹ is hydrogen,
R¹⁰ is C₁₋₈alkyl or C₃₋₁₀cycloalkyl; aryl or arylalkyl;
R¹¹ is hydrogen, C₁₋₈alkyl, C₃₋₁₀cycloalkyl, C₃₋₁₀heterocycloalkyl, C₃₋₁₀aryl or C₃₋₁₀arylalkyl.
T¹ is alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl;
T² and T³ are each independently a single bond, -T⁸-S(O)ₜ-T⁹-, -T⁸-C(O)-T⁹-, -T¹⁸-C(S)-T⁹-, -T⁸-O-T⁹-, -T⁸-S-T⁹-, -T⁸-O—C(O)-T⁹-, -T⁸-C(O)ₜT⁹-, -T⁸-C(═NT¹⁰)-T⁹- or -T⁸-C(O)—C(O)-T⁹-;
T⁵, T⁶, T⁷, T⁸ and T⁹ are independently hydrogen, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alky, each group optionally substituted where valence allows by one to three groups independently selected from halo, cyano, nitro, OH, oxo, —SH, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl, —OT¹¹, —ST¹¹, —C(O)ₜH, —C(O)ₜT¹¹, —O—C(O)T¹¹, TC(O)ₜN(T¹²)T¹¹, —SO₃H, —S(O)ₜT¹¹, S(O)ₜN(T¹²)T¹¹, -T¹³-NT¹¹T¹², -T¹³-N(T¹²)-T⁴-NT¹¹T²², -T¹³-N(T¹¹)-T¹²-T¹¹ and -T¹³-N(T¹⁸)-T¹⁴-H; or
T8 and T⁹ are each independently a single bond, alkylene, alkenylene or alkynylene;
T¹¹ is alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl;
T¹² is halo, cyano, nitro, OH, oxo, —SH, alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocyclo)alkyl, heteroaryl or (heteroaryl)alkyl, —C(O)ₜH or —SO₃H;
T¹³ and T¹⁴ are each independently a single bond, —S(O)ₜ—, —C(O)—, —C(S)—, —O—, —S—, —O—C (O)—, —C(O)ₜ—, —C(═NT¹³)— or —C(O)—C(O)—; and
t is 1 or 2.

More preferred methods/compounds use/are:
A is hydrogen, deuterium, methyl, —CH₂(CH₃)₂, —(CH₂)₂(CH₃)₂, —CH(CH₃)CH₂(CH₃), —(CH₂)OH, hydroxyethyl, —(CH₂)₂SCH₃, —CH₂SH, phenyl, —CH₂(phenyl), —CH₂(p-hydroxyphenyl), —CH₂(indole), —(CH₂)C(O)NH₂, —(CH₂)₂C(O)NH₂, —(CH₂)₂C(O)OH, —CH₂C(O)OH, —(CH₂)₄NH₂, —(CH₂)₃(═NH)CNH₂ or —CH₂(imidazole).

Especially preferred methods/compounds use/are:
A is —CH(CH₃)CH₂(CH₃), phenyl, CH₂(phenyl) or —CH₂(2-indole).

Also, especially preferred methods/compounds use/are:
R⁸ is hydrogen and the configuration about the carbon marked with the * is S, provided A is not H. Also preferred: R8 is deuterium and the configuration about the carbon marked with the * is S, provided A is not H or deuterium.

Other preferred methods/compounds use/are:
R1 and R⁵ are both isopropyl; and/or R⁶R⁷ and R⁹ are all hydrogen; and/or Z is CH₂(phenyl), —C(O)₂H or —C(O)₂C₁₋₈alkyl.

Some example embodiments of Formula (IV)

In the following scheme, Scheme X, all reactants are commercially available e.g. Compound 1 from Matrix Scientific, Columbia, SC, USA, Compound 2 from Oxchem Corporation, IL, USA, Compound 4A from Astatech Inc., Bristol PA, USA, Compound 4B from Apollo Scientific Ltd., Stockport, UK.

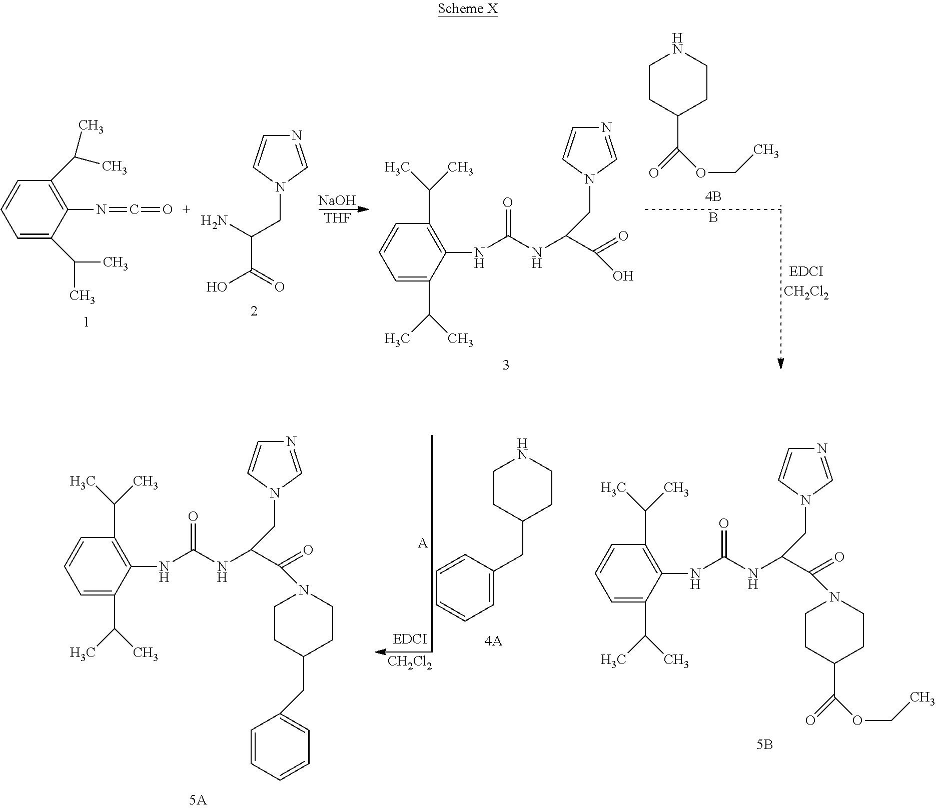

Using Scheme X, above, with different amino acids as the Compound 2 input, gives different Compound 5 products. Non-limiting illustration follows: Compound 2b is available from Aurora Fine Chemicals LLC, San Diego, USA, Compounds 2c, 2d, 2f (CAS number: 54793-54-3), 2g (CAS: 136056-01-4), 2i (L-lysine), 2j (CAS Number: 169524-86-1), 2k (607665 ALDRICH) are available from Sigma-Aldrich, Compound 2h (CAS: 91037-48-8) from Cambridge Isotope Laboratories:

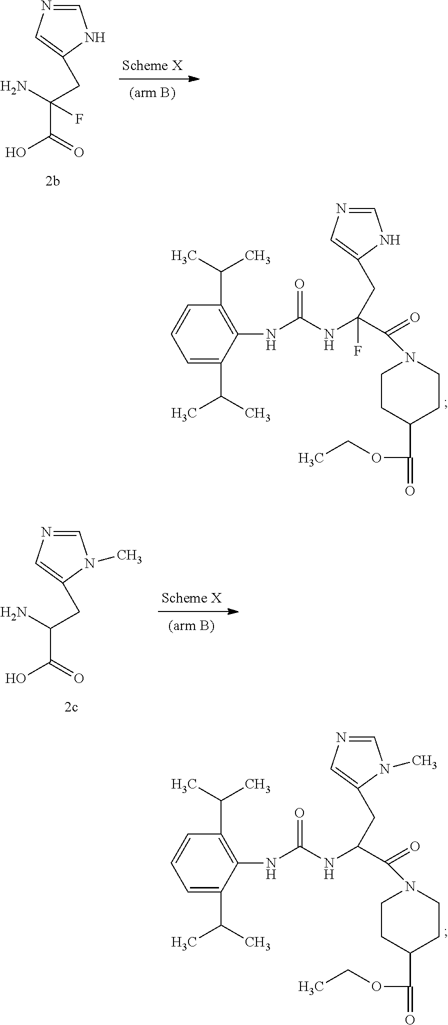 [SMILES: NC(F)(Cc1cnc[nH]1)C(=O)O ; CCOC(=O)C1CCN(C(=O)C(F)(Cc2cnc[nH]2)NC(=O)Nc2c(C(C)C)cccc2C(C)C)CC1 ; Cn1cncc1CC(N)C(=O)O ; CCOC(=O)C1CCN(C(=O)C(Cc2cncn2C)NC(=O)Nc2c(C(C)C)cccc2C(C)C)CC1]

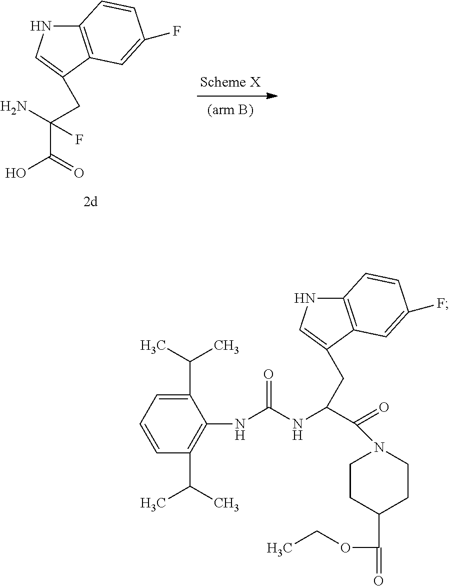 [SMILES: NC(F)(Cc1c[nH]c2ccc(F)cc12)C(=O)O ; CCOC(=O)C1CCN(C(=O)C(Cc2c[nH]c3ccc(F)cc23)NC(=O)Nc2c(C(C)C)cccc2C(C)C)CC1]

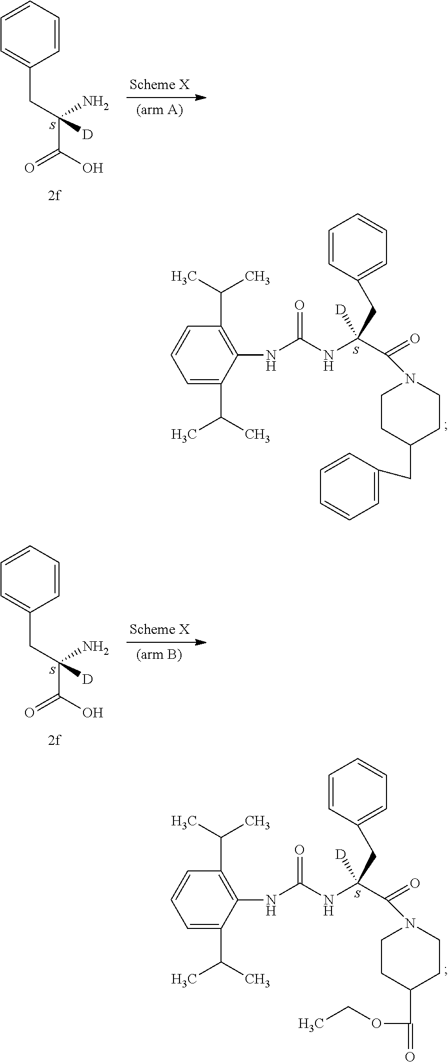

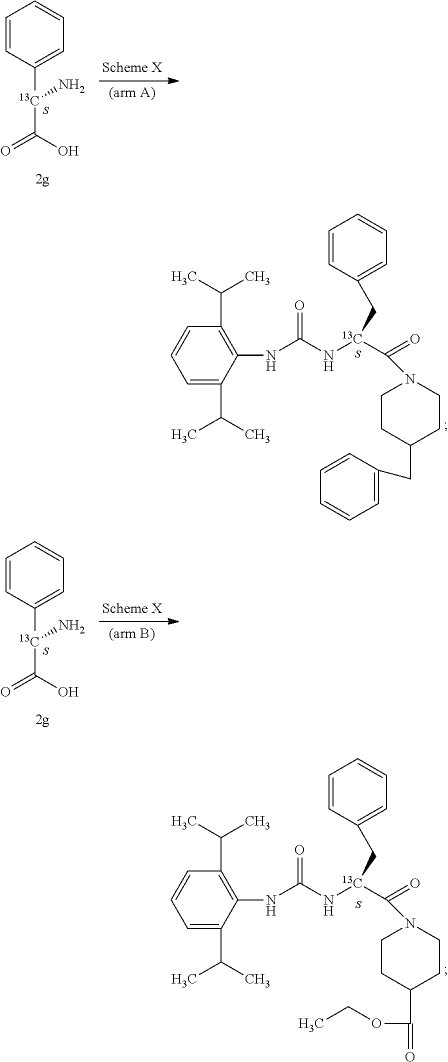

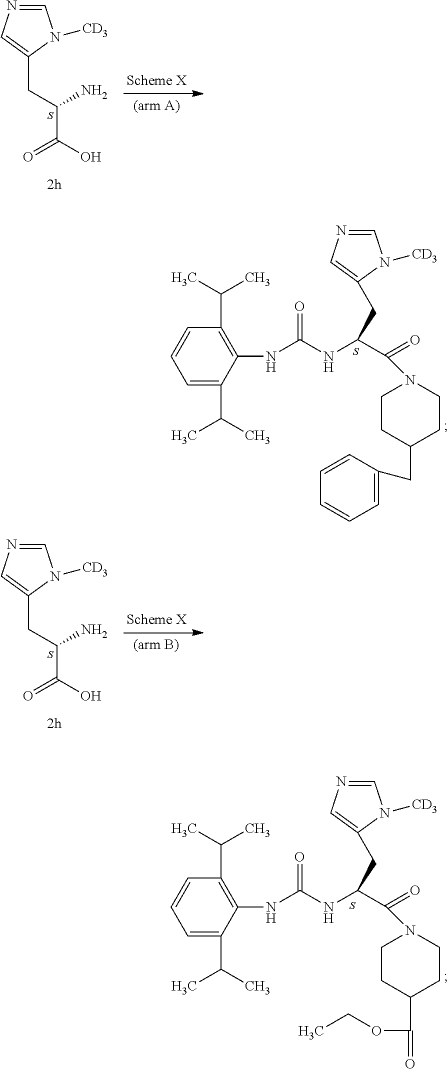

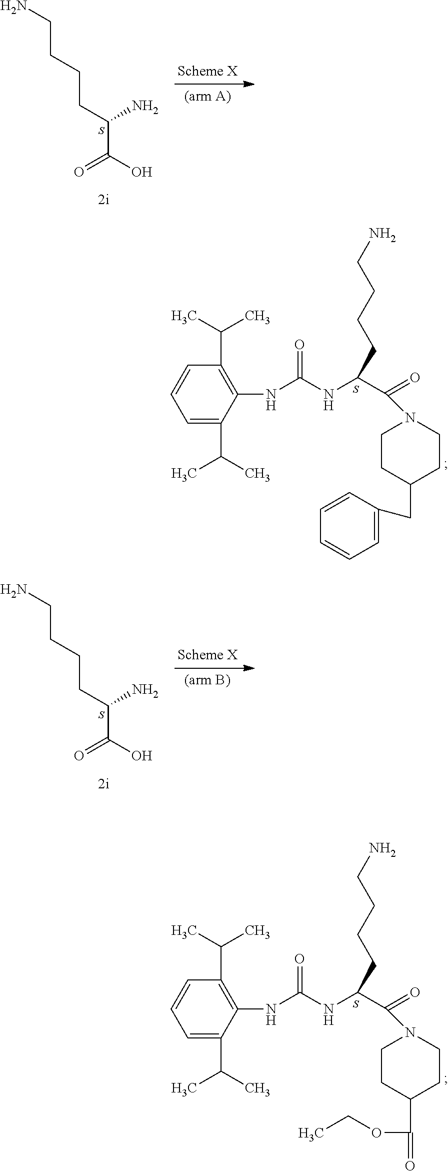

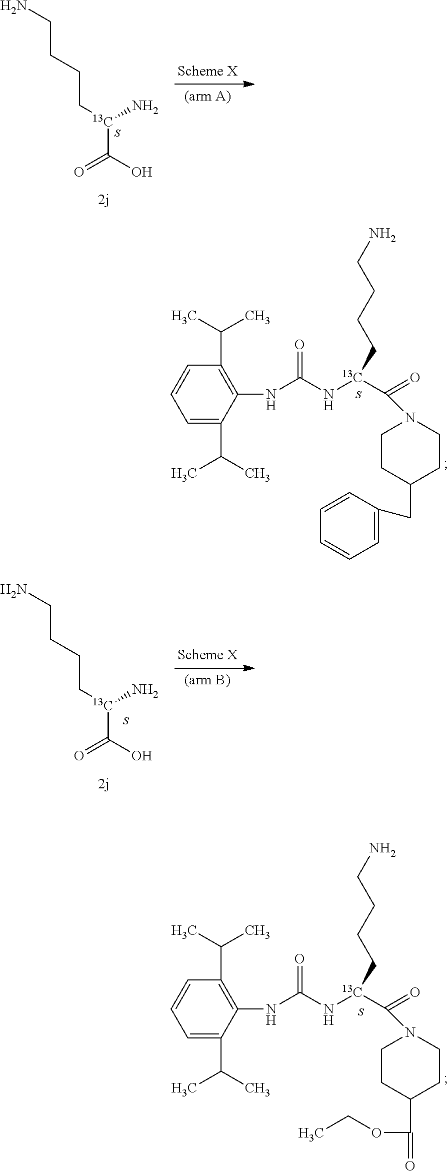

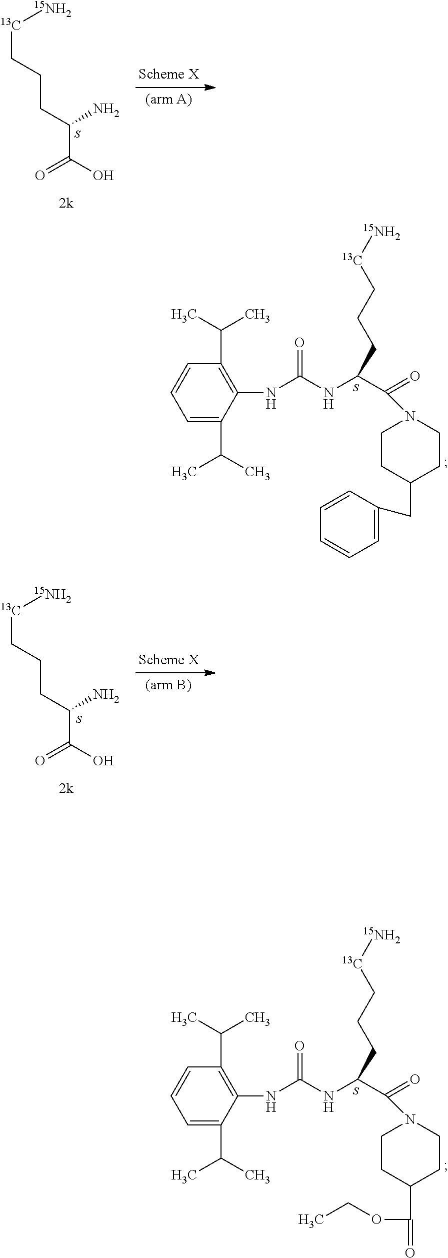

In preferred embodiments, the S stereoisomer product of Scheme X is in enantiomeric excess, optionally because the Compound 2 input into Scheme X has enantiomeric excess of S stereoisomer. To illustrate, with histidine (available from Sigma-Aldrich) as Compound 2 input,

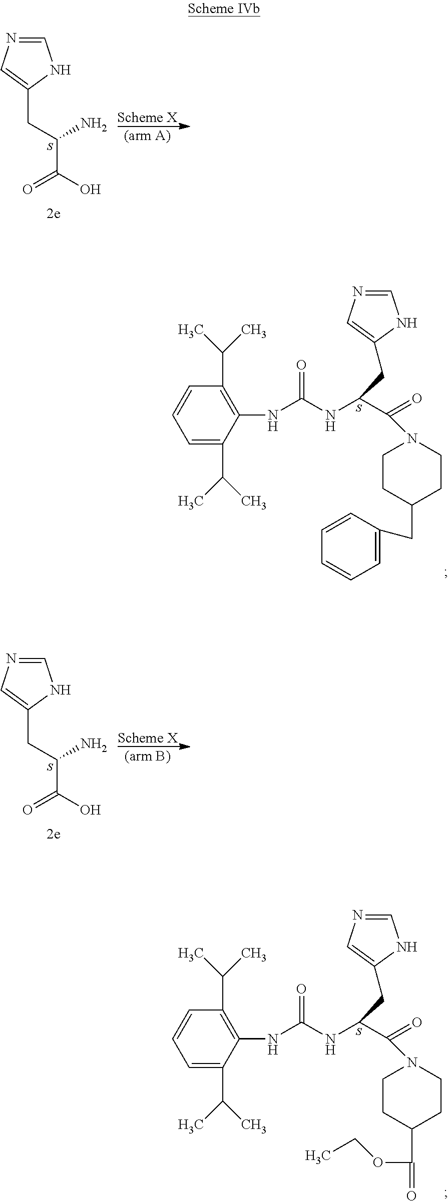

Where the amino acid side chain contains an NH group it can optionally be protected as a first step, preferably using a protecting group with some degree of specificity for an amine over an alcohol group, and for a secondary (NH) over primary (NH₂) amine, wherein greater specificity is more favoured. Or using an amine protecting group (or chemical reaction/modification e.g. [non-limiting] refer [149-150]) with some degree of specificity for a primary over secondary amine, then protecting the secondary amine with a different protecting group, then removing the first protecting group (or reversing the chemical reaction/modification of primary amine) with conditions that don't remove the second protecting group. Thence a protection group on the NH group of the amino acid side chain, and not on the NH₂ and OH of the amino acid, is extant. This judicious use of protecting groups is inherent to one of the art e.g. refer Greene et al., Protective Groups in Organic Synthesis, 3^{rd} Ed., Wiley-Interscience, 1999 (or later edition). Alternatively one can use an amino acid starting material with desirable protections incorporated, for example the starting material, a protected form of L-histidine (CAS: 274927-61-6), in the scheme below is available from multiple suppliers on www.Labnetwork.com e.g. Astatech Inc., Bristol PA, USA

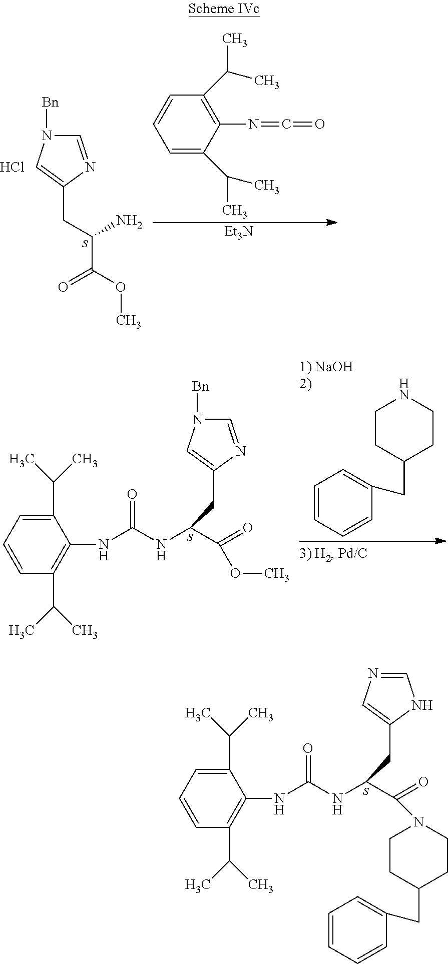

An illustrating, non-limiting, judicious use of protecting groups is shown below, wherein Compound 1 is available from Sigma-Aldrich (609226) and the remaining starting compounds from suppliers listed on www.labnetwork.com: Compound 2 (Fluorochem, Hadfield, Derbyshire, UK), Compound 5 (Astatech Inc., Bristol PA, USA), Compound 9 (Matrix Scientific, Columbia, SC, USA) and Compound 12 (Alfa Aesar, Shanghai, China).

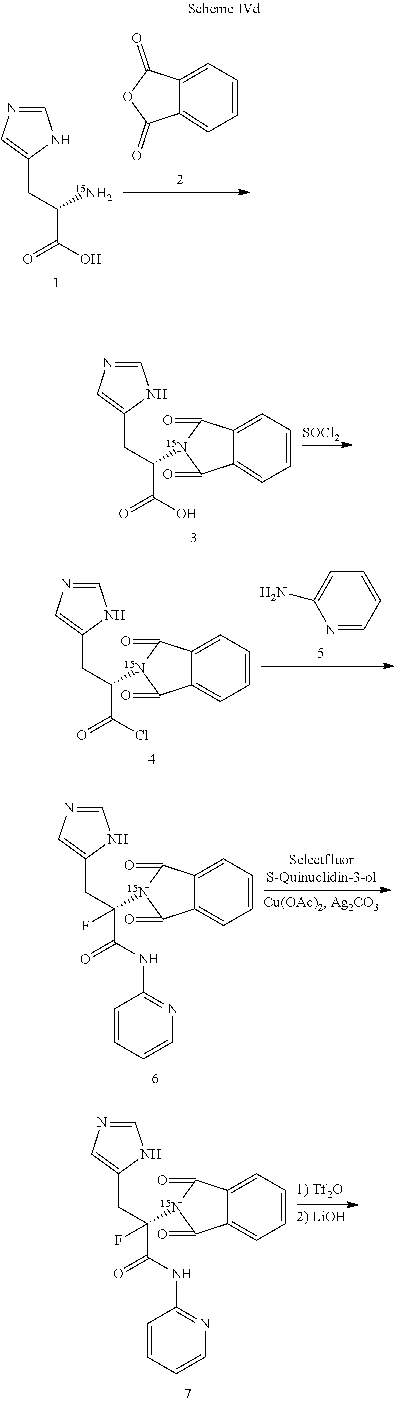

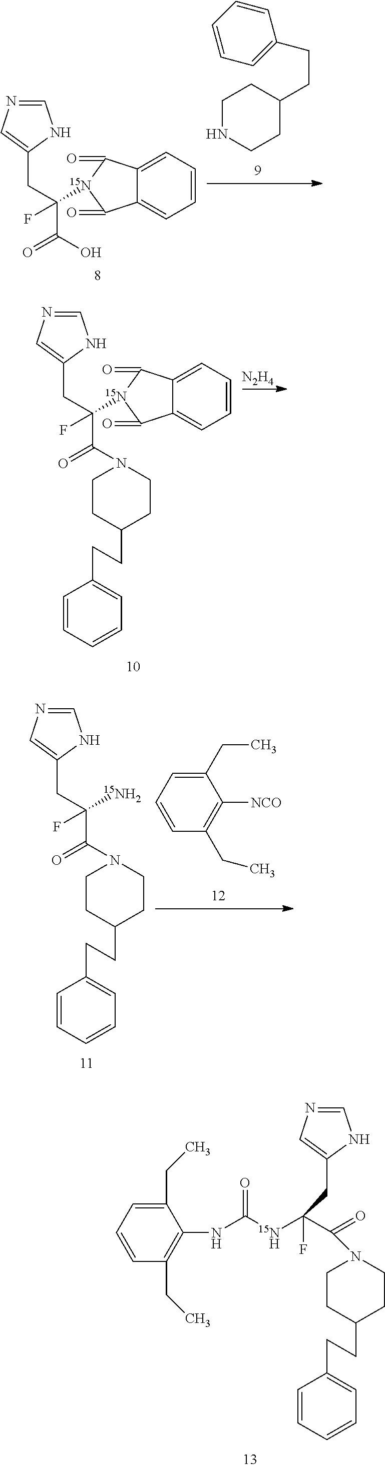

For Scheme IVe below, starting compounds can be sourced from suppliers listed on labnetwork.com, e.g. Compounds 1, 2, 4 can be sourced from Astatech Inc., Bristol PA, USA.

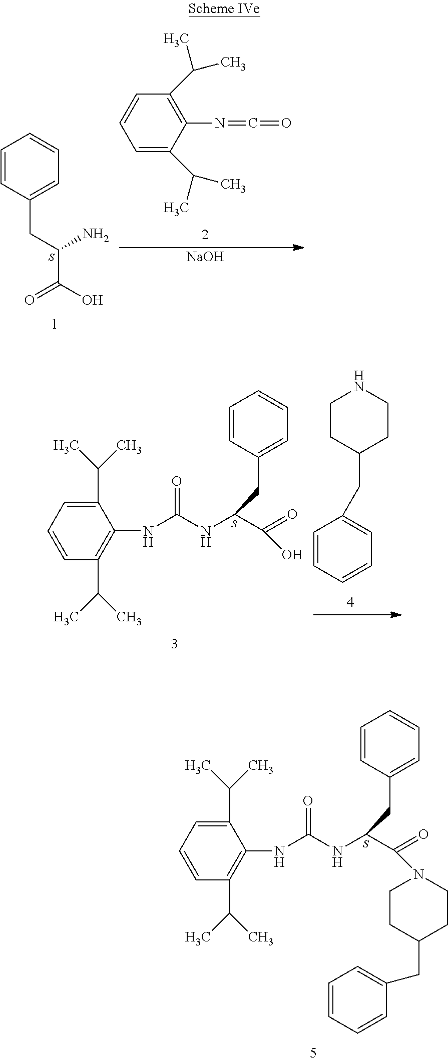

For Scheme IVf below, starting compounds can be sourced from suppliers listed on labnetwork.com, e.g. Compounds 1, 2, 4, 7 can be sourced from Astatech Inc., Bristol PA, USA.

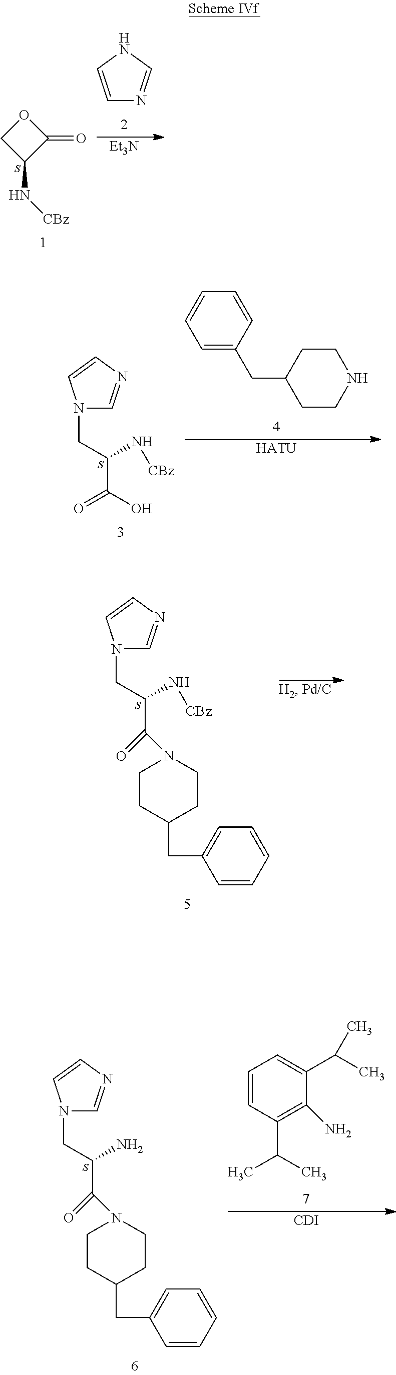

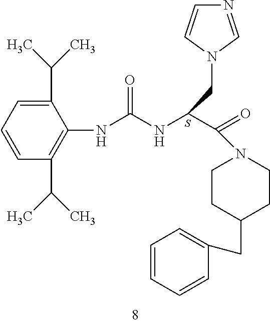

Other example embodiments (each structure a separate embodiment):

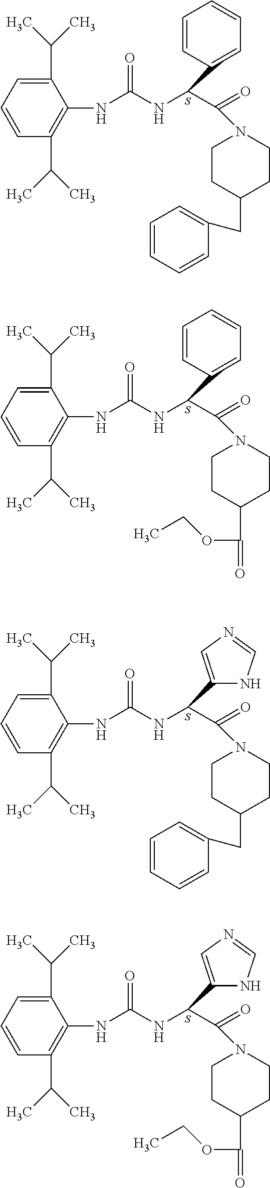

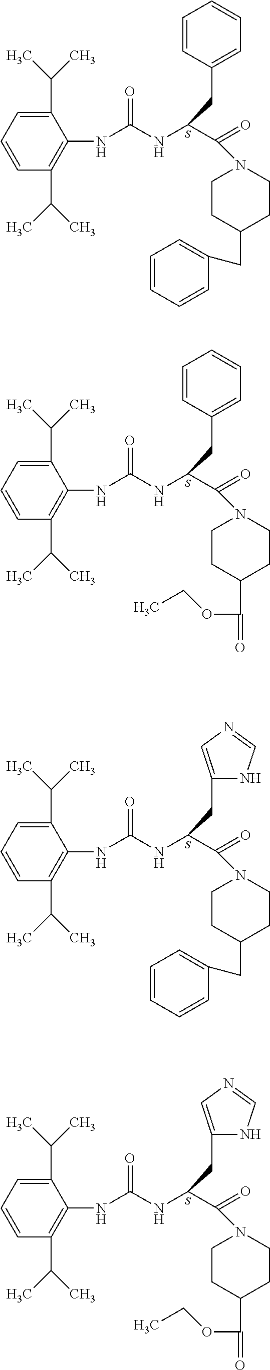

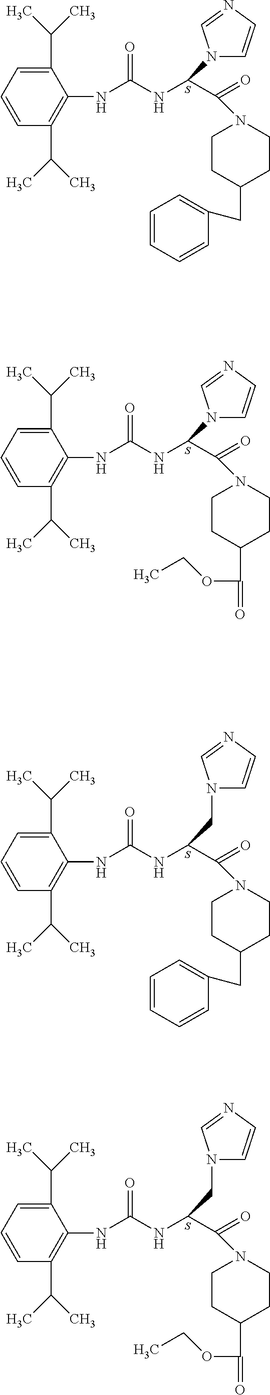

### Example (V)

### Formula (V)

Herein incorporated by reference, in its entirety, is Formula (V), and example embodiments thereof, from PCT/EP2018/069175 (published as WO2019/012149A1).

### Example (VI)

Encompassed by this embodiment are methods of treating a subject suffering from cancer by administering an effective amount of at least one compound of Formula (VI) or a pharmaceutically-acceptable salt, solvate, hydrate or prodrug thereof, or a pharmaceutical composition(s) comprising one or compounds of Formula (VI).

### Summary of Formula (VI)

This disclosure embodiment relates to compounds having the following formula: Formula (VI)

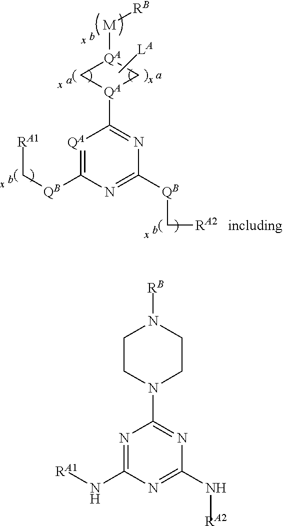

or a pharmaceutically-acceptable salt, solvate, hydrate or prodrug thereof, wherein:
each Q^{A} is independently selected from N and CH;
each Q^{B} is independently selected from O, S, Se, NH, CH₂, NR^{W}, PR^{W}, BR^{W}, C(R^{W})₂ and Si(R^{W})₂;
each M is independently selected from O, S, Se, NH, CH₂, NR^{W}, PR^{W}, BR^{W}, C(R^{W})₂ and Si(R)₂;
each R^{W} is independently selected from hydrogen, deuterium, halogen (e.g. F), alkyl, or substituted alkyl (non-limiting examples: CF₃, CCl₃), or deuterated alkyl (non-limiting example: CD3), or aminoalkyl, or thioalkyl, or alkoxy, or halogen, or haloalkyl, or haloalkoxy;
x^{a} is independently at each point of use selected from 1, 2, 3, 4, or 5; x^{b} is independently at each point of use selected from 0, 1, 2, 3, 4, or 5; LA represents 0-5 optional substituents on the ring independently selected from alkyl, substituted alkyl, deuterated alkyl, aminoalkyl, thioalkyl, alkoxy, halogen, haloalkyl, haloalkoxy, or any atom or isotope permitted by valence (including any accompanying hydrogens by valence e.g. (non-limiting) OH, NH₂, SH, SiH₃, PH₂ etc.);
R^{A1} and R^{A2} are each independently selected from the groups

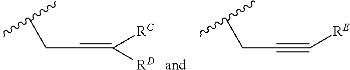

wherein R^{C} and R^{D} are each independently selected from hydrogen, deuterium, halogen and alkyl, and
wherein R^{E} is hydrogen, deuterium, halogen or alkyl;
R^{B} is selected from R^{B1}, hydrogen and deuterium;
wherein R^{B1} is selected from phenyl, benzyl, heteroaryl, pyridyl, pyrimidyl and pyrazinyl optionally substituted independently with one or more substituents of R^{B2};
wherein each R^{B2} is independently selected from halogen, alkyl, substituted alkyl, deuterated alkyl, alkoxy, nitro, amino, methoxy, haloalkyl, polyhalogen alkyl, aminoalkyl, thioalkyl, alkoxy, haloalkoxy, and any atom or isotope permitted by valence (including any accompanying hydrogens by valence e.g. (non-limiting) OH, NH₂, SH, SiH₃, PH₂ etc.); or R^{B} is a phenylalkyl of the formula:

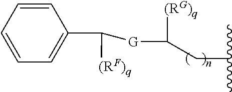

wherein R^{F} and R^{G} are hydrogen or alkyl, G is a carbon-carbon double bond or a carbon-carbon single bond, n is 0 or 1 and q is 0 or 1 provided that where q is 0, G is a carbon-carbon double bond and where q is 1, G is a carbon-carbon single bond, or R^{B} is a diphenylalkyl of the formula

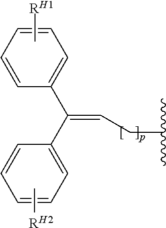

wherein R^{H1} and R^{H2} each independently represent 1-5 optional substituents on each ring, and wherein each R^{H1} and R^{H2}, when present, is independently selected at each point of use from hydrogen, deuterium, halogen, alkyl, substituted alkyl, deuterated alkyl, alkoxy, nitro, amino, methoxy, haloalkyl, polyhalogen alkyl, aminoalkyl, thioalkyl, alkoxy, haloalkoxy, and any atom or isotope permitted by valence (including any accompanying hydrogens by valence e.g. (non-limiting) OH, NH₂, SH, SiH₃, PH₂ etc.), and p is 0, 1, 2 or 3; or R^{B} is the group

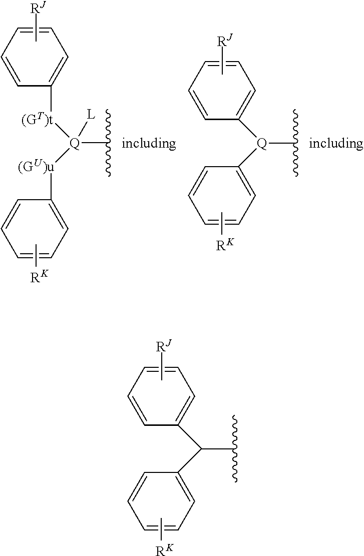

Wherein G^{T} and G^{U} are each independently selected from a single bond, O, S, NR^{V} or C(R^{V})₂, wherein each R^{V} is independently selected from hydrogen, deuterium, alkyl, substituted alkyl (non-limiting examples: CF₃, CCl₃), deuterated alkyl (non-limiting example: CD₃), aminoalkyl, thioalkyl, alkoxy, halogen (e.g. F), haloalkyl, haloalkoxy;
u and t are each independently selected from 0, 1, 2, 3 and 4;
Q is C, CH or N, R^{J} and R^{K} each independently represent 1-5 optional substituents on each ring, and wherein each R′ and each R^{K}, when present, is independently selected from deuterium, halogen, alkyl, substituted alkyl, deuterated alkyl, alkoxy, haloalkoxy, methoxy, nitro, amino, aminoalkyl, thioalkyl, haloalkyl, polyhalogen alkyl, and any atom or isotope permitted by valence (including any accompanying hydrogens by valence e.g. (non-limiting) OH, NH₂, SH, SiH₃, PH₂ etc.);
L is absent (when Q is N), alkyl, or substituted alkyl, or deuterated alkyl, or aminoalkyl, or thioalkyl, or alkoxy, or halogen, or haloalkyl, or haloalkoxy, or hydroxyalkyl, or any atom or isotope permitted by valence (including any accompanying hydrogens by valence e.g. (non-limiting) OH, NH₂, SH, SiH₃, PH₂ etc.).

In some embodiments, when one or both of R^{J} and R^{K} is alkoxy, this alkoxy group may be methoxy.

It is to be understood that in the compounds of general Formula (VI), wherein R^{A1} and/or R^{A2} are alkenyl moieties having different substituents at the position RC and R^{D}, that compound may exist in cis or trans isomeric forms both of which are considered to be within the scope of the present disclosure. All isotopic, including radionuclide, forms of Formula (VI) are within the scope of the present disclosure.

### Some Preferred Embodiments of Formula (VI)

For Formula (VI), the symbols RC and R^{D}, as defined in subgroups R^{A1} and R^{A2}, may be hydrogen, halogen (suitably fluorine, chlorine or bromine), alkyl, suitably “lower alkyl” (herein now defined) having from 1 to 5 carbon atoms, such as methyl, ethyl, propyl, isopropyl, butyl, isobutyl, tert butyl, pentyl and the like, most preferably methyl;
and the moiety RE may be hydrogen, or lower alkyl having from 1 to 5 carbon atoms such as methyl, ethyl, propyl, butyl, or pentyl, most suitably methyl.

The subgroup R^{B} may be hydrogen; phenyl; or substituted phenyl. The substituted phenyl group may include one or more of the preferred substituents in any of the available positions for substitution, however, mono substitution in the 4-position of the phenyl nucleus is especially preferred. Suitable substituents for the phenyl nucleus include halogen, preferably fluorine, chlorine or bromine; lower alkyl, lower alkoxy, and poly halogen lower alkyl (i.e, substituted alkyl) wherein the alkyl moiety contains from 1 to 5 carbon atoms, especially preferred however are methyl, methoxy, trifluoromethyl, nitro and amino. Where the subgroup R^{B} represents substituted pyridyl, substituted pyrimidyl, or substituted pyrazinyl, the substituting group may be located on one or more of the available carbon atoms in the nucleus, and may be the same or different. Preferred among the substituting groups are lower alkyl or lower alkoxy having from 1 to 5 carbon atoms such as methyl, ethyl, butyl or penty; or methoxy, propoxy, butoxy or pentoxy. Where the moiety R^{B} represents substituted benzyl, the benzyl moiety may be substituted in one or more of the available positions on the phenyl nucleus thereof. Among the preferred substituents are halogen (suitably fluorine, chlorine or bromine), lower alkoxy having from 1 to 5 carbon atoms, especially preferred is methoxy and most preferred being di- and tri-methoxy; or alkylenedioxy suitably lower alkylenedioxy such as methylenedioxy, ethylenedioxy, propylenedioxy and the like, most suitably, the alkylenedioxy moiety is attached across the 3- and 4-positions of the phenyl nucleus, although the bridging of other carbon atoms in the phenyl nucleus is to be considered within the scope of the present disclosure.

The moieties R^{F} and R^{G} may be hydrogen, or lower alkyl of 1 to 5 carbon atoms, most preferred however being methyl.

The groups R^{H1} and R^{H2} may be independently hydrogen, or halogen suitably fluorine, chlorine or bromine.

Preferred embodiments of Formula (VI) include wherein R^{C} and R^{D} are methyl, R^{E} is methyl and R^{B} is selected from chlorophenyl, methylphenyl, methoxyphenyl, trifluorophenyl, chlorophenyl, dimethoxybenzyl, trimethoxybenzyl, methylenedioxybenzyl and ethylenedioxybenzyl.

In some embodiments R^{B} is the group

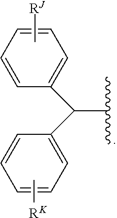

In some embodiments, R^{B} is the group

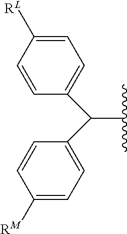

wherein R^{L} and R^{M} are each independently selected from halogen, alkyl, alkoxy, nitro, amino and polyhalogen alkyl.

### Almitrine as an Anti-Cancer Medicine

Almitrine is especially valuable against a cancer that can/does disrupt/impair breathing (e.g. lung cancer, cancer in the lung, primary lung cancer or a cancer that has spread/metastasized to the lungs and/or chest area, mesothelioma, cancer causing one or more of pleural effusion, pulmonary oedema, ascites) and/or that causes dyspnea (50-70% of cancer patients have this symptom at some time during their illness, 90% for patients with advanced lung cancer, source: https://www.cancerresearchuk.org/about-cancer/coping/physically/breathing-problems/shortness-of-breath) and/or that reduces O₂ delivery to tissues.

In rats, radioactively labelled 14C-almitrine is especially accumulated in the lungs [151], and to a lesser degree, the caratoid body. Therefore, in some disclosure embodiments, almitrine or a pharmaceutically-acceptable salt, solvate, hydrate or prodrug thereof is especially suited, without limitation, to treating/ameliorating/preventing/combating
(a) lung cancer(s) such as, without limitation, Small Cell Lung cancer (SCLC), Non-Small Cell Lung Cancer (NSCLC), small-cell lung carcinoma (SCLC), non-small-cell lung carcinoma (NSCLC, including, without limitation, adenocarcinoma of the lung, bronchioloalveolar lung cancer, bronchioloalveolar carcinoma, squamous-cell carcinoma of the lung, large-cell lung carcinoma, pleomorphic, carcinoid tumor, salivary gland-like carcinoma, unclassified carcinoma, rhabdoid carcinoma, sarcomatoid carcinoma, adenosquamous carcinoma, papillary adenocarcinoma, giant-cell carcinoma, an admix of NSCLC types, “not otherwise specified” type), combined small-cell lung carcinoma (c-SCLC), pancoast tumors, carcinoid tumors, bronchial gland carcinomas, sarcomatoid carcinomas and non-carcinomas (such as, without limitation, sarcoma, lymphoma, immature teratoma, melanoma), a cancer(s) listed in [152], cancer(s) in a bronchus/bronchi, bronchial adenoma;
(b) cancer(s) of the caratoid body, such as carotid paraganglioma (carotid body tumor), and/or a liver cancer(s) such as hepatocellular carcinoma, cholangiocarcinoma, hepatoblastoma and/or a kidney cancer(s) such as renal cell carcinoma (RCC), renal oncocytoma, transitional cell carcinoma (TCC), squamous cell carcinoma, juxtaglomerular cell tumor (reninoma), angiomyolipoma, Bellini duct carcinoma, clear-cell sarcoma of the kidney, mesoblastic nephroma, metanephric adenoma, cystic nephroma, Wilms' tumor, mixed epithelial stromal tumor and/or a heart cancer(s) (primary and/or secondary) such as papillary fibroelastoma, rhabdomyoma, angiosarcoma, teratoma, cystic tumour of the atrioventricular nodal region and/or a cancer of the adrenal gland such as adrenocortical adenoma, adrenocortical carcinoma, neuroblastoma, pheochromocytoma, and paraganglioma.

An embodiment(s) of this disclosure is administering a compound of Formula (VI), optionally almitrine, and/or a pharmaceutically-acceptable salt, solvate, hydrate or prodrug thereof to a subject with cancer, optionally lung cancer and/or mesothelioma of the lung, wherein this subject has difficulty breathing and/or has low blood pO₂ (hypoxia) and/or high blood pCO₂ and/or Chronic Obstructive Pulmonary Disease (COPD) and/or Acute Respiratory Distress Syndrome (ARDS) and/or Severe Acute Respiratory Distress Syndrome (SARDS) and/or Severe Acute Respiratory Syndrome (SARS) and/or virus and/or coronavirus (e.g. Betacoronavirus, SARS-COV, SARS-COV-2 [cause of COVID-19], MERS-COV, HCoV-NL63, coronavirus that can infect humans etc.) infection and/or is undergoing (or will undergo or has undergone) surgery including anti-cancer surgery including surgery to remove lung cancer including surgery to remove part or the entirety of a lung (pneumonectomy) including open-chest thoracic surgery (thoracotomy) including open-chest one lung ventillation, optionally in co-administration with (e.g. inhalation of) Nitric Oxide, NO (illustratively, not restrictively, NO at 10 parts per million [p.p.m]) and/or hyperbaric O₂ therapy (oxygen therapy/supplemental oxygen) and/or mechanical/assisted ventilation (artificial invasive/non-invasive assistance to support breathing) and/or extracorporeal membrane oxygenation and/or anti-viral treatment(s) (e.g. anti-viral treatment for a coronavirus infection). Many cancer (e.g. some chemo-/radio-) therapies impair the immune system, as can cancer itself (e.g. leukemia e.g. AML), and so subjects with cancer, especially those undergoing immunocomprimising cancer treatment(s) (e.g. alkylating/-platin chemotherapy), are at increased risk during an epidemic/pandemic. Almitrine does not immunosuppress and so is a favoured anti-cancer therapy for use during an epidemic/pandemic, when cancer patients require as much immune function as possible. Moreover, in the case of a coronavirus (e.g. SARS-COV-2), which can cause breathing difficulty, the respiratory stimulation that almitrine causes is very clinically useful (almitrine increases pO₂, and decreases pCO₂, in the blood and tissues [153, 154]). Indeed, a danger of a coronavirus (e.g. SARS-COV-2) is that it can cause Severe Acute Respiratory Distress Syndrome (SARDS) [155], wherein almitrine is actually used (and has been used for decades) clinically for treating Acute Respiratory Distress Syndrome (ARDS) [156], and more generally for “hypoxaemia and hypercapnia associated with alveolar hypoventilation” [157], showing efficacy with Severe Acute Respiratory Distress Syndrome (SARDS) [158]. An effective amount (e.g. therapeutically effective amount) of almitrine (and/or a pharmaceutically-acceptable salt, solvate, hydrate or prodrug thereof) can optionally be administered to a subject as a supplement to their oxygen and/or nitric oxide therapy and/or mechanical/assisted ventilation and/or extracorporeal membrane oxygenation, and/or as an assist to transitioning a subject off one or more of these, and/or as a substitute for one or more of these, and/or rendering a subject's hospitalization shorter (the subject's need for a hospital machine(s)/care is delayed and/or shortened) or unrequired. This is especially useful during an epidemic/pandemic when there might not be enough hospital machines/beds for everyone with need. Moreover, hospitals can be a hub of coronovirus spread. Disproportionally to those least fit to endure it, and to key healthcare workers also, which is a vicious combination. So keeping subjects with (or suspected to have, or at risk of, or particularly vulnerable to) coronavirus out of hospital as much as possible, either by delaying their admission, expediting their discharge or mitigating their need for hospitalization, is valuable. Also, being a drug with such a low side-effect profile, especially when taken short-term (daily for <11 months) [159], almitrine can be taken with atypically limited medical oversight if access to a Doctor(s) becomes atypically limited. Also contemplated by this disclosure is the administration of almitrine (and/or a pharmaceutically-acceptable salt, solvate, hydrate or prodrug thereof), and/or other respiratory stimulant drug (non-limiting e.g. doxapram and/or a pharmaceutically-acceptable salt, solvate, hydrate or prodrug thereof), to a subject, without cancer, but who has (or is suspected to have, or who is at risk of, or particularly vulnerable to) a coronavirus (e.g. SARS-COV-2) infection, optionally in co-therapy with Nitric Oxide (NO) treatment (e.g. via inhalation) and/or an anti-viral drug(s) (non-limiting e.g. one or more of Remdesivir, Lopinavir, Ritonavir), optionally wherein almitrine (and/or a pharmaceutically-acceptable salt, solvate, hydrate or prodrug thereof) and an anti-viral drug(s) is in the same pharmaceutical composition. Almitrine can be used to transition subjects off mechanical/assisted ventilation [157], and so if employed during the COVID-19 crisis, and/or other virus/coronavirus crisis, it can free up ventilator machines quicker, and head off the need for a ventilator machine in more minor cases, buying time to be allocated a ventilator machine in more serious cases. This will ease the most dangerous (projected) pinch point of the COVID-19 crisis: not enough ventilator machines for those that need them. Almitrine or a pharmaceutically-acceptable salt, solvate, hydrate or prodrug thereof is especially suited to treating lung cancer because it exerts anti-cancer activity, thence fundamentally treating the symptoms of lung cancer, and concurrently directly treats the shortness of breath/beathlessness symptom of lung cancer. Without limitation to any mechanism(s), the former action is by almitrine effect upon ATP synthase in cancer cells, the latter action is by almitrine effect upon BK potassium channels in the caratoid bodies, wherein this latter action also exerts anti-cancer activity, adding/potentiating with the first, because it increases tissue pO₂ and thence ROS production. Elegantly, almitrine both increases ROS (by increasing blood and tissue pO₂) and decreases ROS mitigation, especially in cancer cells (by slowing F₁F₀ ATP hydrolysis).

Physiologically, hypoxia in part of a lung (perhaps because of lung damage and/or fluid in that part) causes vasoconstriction in this lung part (“hypoxic pulmonary vasoconstriction”). So that more blood can flow instead to other lung parts that actually have appreciable O₂ to deliver to the blood. This vasoconstriction increases pulmonary tension. Pharmacologically, almitrine helps and increases this process. So increasing pO₂, and decreasing pCO₂, in the blood and tissues. Inherently increasing pulmonary tension, which can be problematic [160]. Breathable Nitric Oxide (NO) can be co-administered with almitrine [177]. NO is a vasodilator. NO, when incorporated in the breathing mixture, only reaches the lung parts that O₂ reaches. So, it only vasodilates the lung parts that are well ventillated with O₂. So,
(1) Almitrine specifically vasoconstricts only hypoxic lung regions, shunting more blood to well ventillated lung regions.
(2) Breathed NO only reaches well ventillated lung regions and so specifically vasodilates only well ventillated lung regions.

Thence points (1) and (2) additively increases PaO₂ [177]. Incidentally, whilst point (1) increases pulmonary tension, point (2) decreases pulmonary tension, and so with almitrine and NO co-administration there is partial/complete cancelling of their opposing pulmonary tension effects, with concurrent addition of their beneficial increase in PaO₂ [177]. Componentry to this disclosure is to co-administer almitrine (and/or a pharmaceutically acceptable salt, solvate, hydrate or prodrug thereof; non-limiting e.g. intravenous and/or oral) with breathed NO to a subject, optionally wherein this subject has cancer, wherein the breathed NO permits a higher almitrine dose(s), conferring greater anti-cancer activity, to be administered to the subject because the breathed NO counteracts an almitrine conferred increase in pulmonary tension. Wherein a higher NO dose can permit a higher almitrine dose. Also componentry to this disclosure is to co-administer almitrine (and/or a pharmaceutically acceptable salt, solvate, hydrate or prodrug thereof; non-limiting e.g. intravenous and/or oral) and breathed NO, optionally along with assisted ventillation and/or hyperbaric oxygen, to a subject infected with at least one coronavirus, optionally SARS-COV-2, optionally to treat/ameliorate/prevent/combat a coronavirus driven/associated/correlated respiratory disorder(s), optionally Severe Acute Respiratory Syndrome (SARS) and/or Severe Acute Respiratory Distress Syndrome (SARDS). Also contemplated by this disclosure is almitrine (and/or a pharmaceutically acceptable salt, solvate, hydrate or prodrug thereof) administered to a subject by their inhalation/breathing, optionally along with Nitric Oxide (NO) and/or O₂ and/or a breathing mixture that can sustain a subject.

Oral almitrine dimesylate (100-200 mg) was administered to human COPD patients daily for a year [151]. In a minority, peripheral neuropathy started to manifest (5 times higher incidence in almitrine than control group) by 7 months on average. Subjects with greatest plasma [almitrine] were most at risk. Averaged, those presenting peripheral neuropathy in the study year had plasma [almitrine] of 344 and 617 ng/ml at 3 and 12 months respectively, those that didn't had plasma [almitrine] of 249 and 387 ng/ml at 3 and 12 months respectively. The suggested optimal long term mean (not peak) plasma [almitrine] for treating COPD, without inducing neuropathy, is in the 200-300 ng/ml range [151, 161]. Almitrine has a long half-life in the human body. When daily almitrine intake exceeds daily almitrine elimination from the body, as it does with ≥100 mg oral almitrine dimesylate dosed per day [162], there is fractional compounding of daily doses, which drives higher plasma [almitrine] over time, until an eventual point (between day 90 and day 180 with the 100 mg administered in [162]) when plasma [almitrine] stops increasing and stabilizes. If the almitrine dimesylate daily dose is sufficiently high (≙100 mg and more surely ≥200 mg), peripheral neuropathy can occur in the minority of COPD subjects that eliminate almitrine most poorly, who have greatest compounding of almitrine daily doses, and in which the greatest mean and trough plasma [almitrine] occurs (trough refers to the lowest drug concentration between two doses). Wherein it still typically takes months, on 100-200 mg almitrine dimesylate daily, for this fraction of COPD subjects to accumulate enough almitrine in their body to cause peripheral neuropathy [161]. The majority of COPD subjects don't accumulate such an amount with clinically used almitrine doses (50 to 200 mg oral almitrine dimesylate per day), at least for the length of periods that have been studied, which is months to years. So peripheral neuropathy is a dose-dependent side-effect, wherein the overwhelming majority of COPD subjects administered, even with 200 mg oral almitrine dimesylate daily, don't accumulate sufficient [almitrine] in their body for it to occur. Almitrine administered subjects suffering paraesthesiae/peripheral neuropathy must already have a high almitrine concentration in their body, which is enough to exert significant anti-cancer activity in subjects with cancer. For these subjects, the solution is just to stop/reduce their almitrine daily dosage. The almitrine concentration in their body will then decrease, and the paraesthesiae/peripheral neuropathy with it. Afterwards, optionally, their almitrine dosing can be restarted, optionally at a lower (e.g. daily) dosage. This is a reactionary dosage regime. Alternatively a “proactive dosage regime” can be used. Wherein the administered daily dosage of almitrine, or a pharmaceutically-acceptable salt, solvate, hydrate or prodrug thereof, is decreased over the treatment course, by some function of time since treatment start (non-limiting e.g. 200 mg almitrine dimesylate administered per day for 1st month, 100 mg per day in subsequent month(s)), and/or the course of administration has pauses or rest periods, wherein less or no almitrine is administered (non-limiting example: a repeating cycle of [2 months almitrine administration, 1 month none]). Optionally the almitrine administration dosage/regime can be individually tailored to the almitrine elimination parameters of each individual subject. When deciding the almitrine dosage/regime to administer, the duration of the administration course should be a principal consideration. Wherein if the course is short, e.g. for days to weeks, it is probable that constantly high daily almitrine concentrations (e.g. 200 mg oral almitrine dimesylate per day) will not produce significant side-effects in most to all subjects. Indeed, 400 mg oral almitrine dimesylate per day is safe, in healthy subjects at least [163], at least over the short term. For longer courses of almitrine administration (non-limiting e.g. >3 months), to protect the minority of subjects susceptible to associated peripheral neuropathy, a “proactive dosage regime”, as prior defined, can be used, and/or a lower almitrine dose. Illustratively, in human COPD patients, 75 mg oral almitrine dimesylate administered daily for 6 months produced no adverse effects in any of the study subjects, wherein at study end, the average of the trough (lowest between doses) plasma [almitrine] in the subjects was 302 ng/ml. In human COPD patients, administered 100 mg oral almitrine dimesylate daily for 2 months, then 1 month with no administration, and this cycle was repeated for 1 year, at the end of which the mean plasma [almitrine] was 285 ng/ml, and the authors mention that “there is a close relationship between the almitrine [mean or trough I would specify] plasma level and the occurrence of side-effects such as peripheral neuropathies”, concluding that their regime is safe. In human COPD patients, administered 50 mg almitrine dimesylate twice daily (i.e. 100 mg per day) for 8 weeks and, before the morning dose, plasma [almitrine] was on average across the subjects: 93, 134, 148, 171 ng/ml on days 14, 28, 42, 56 respectively, and 104 ng/ml 2 weeks after the study. In different human COPD patients, administered 100 mg almitrine dimesylate twice daily (i.e. 200 mg per day) for 8 weeks and, before the morning dose, plasma [almitrine] was on average across the subjects: 268, 409, 442, 572 ng/ml on days 14, 28, 42, 56 respectively, and 311 ng/ml 2 weeks after the study. In human COPD patients, over long term, two 50 mg doses of oral almitrine dimesylate per day results in plasma [almitrine] that is 2 to 3 times higher than a single 100 mg oral dose [167], so dividing the daily dose can make a big difference. In human COPD patients, administered 100 mg oral almitrine dimesylate daily for 1 year and at year end mean plasma [almitrine] was 409.35 ng/ml, trough plasma [almitrine] was 301.8 ng/ml (doesn't comment on any side-effects observed or lack thereof, was a purely pharmacokinetic study). In human COPD patients [168], 100 mg oral almitrine dimesylate daily for 1 year increased the occurrence, compared to placebo control, of a nervous system disorder by 4%, paraesthesia by 5% and polyneuropathy by 3.6%.

The way almitrine exerts anti-cancer activity it is suited to treat chemoresistant and/or radioresistant cancer(s) (e.g. lung cancer [s]) because it undermines a mechanism by which these cancers have radio- and/or chemo-resistance. In some embodiments, a compound of Formula (VI), optionally almitrine, or a pharmaceutically-acceptable salt, solvate, hydrate or prodrug thereof is used as an adjuvant or neoadjuvant to another cancer treatment(s) e.g. used as an adjuvant to chemo and/or radiotherapy e.g. used as a chemosensitizer and/or radiosensitizer/radioenhancer. Many conventional [chemo/radio] therapies act against cancer, wholly or in part, by increasing [ROS]. Indeed, radiotherapy and chemotherapy [170, 171, 172] increase [ROS] in cancer cells. A mechanism that cancers use to mitigate and counteract this [ROS] increase is greater F₁F₀ ATP hydrolysis, consuming ATP, which releases glycolysis from ATP feedback inhibition, permitting higher glycolytic and pentose phosphate pathway rate, more NADPH produced and thence greater ROS mitigation. Almitrine slows F₁F₀ ATP hydrolysis, reducing ROS mitigation and this assists the anti-cancer activity of chemo/radio therapy. Almitrine also increases blood and tissue pO₂, increasing ROS production. So, almitrine combats tumor hypoxia, wherein this hypoxia can be a drive to radio-[169] and chemo-[173-174] resistance of cancer. In some embodiments the anti-cancer activity of almitrine or a pharmaceutically acceptable salt, solvate, hydrate, prodrug thereof, synergizes with (potentiates) the anti-cancer activity of an FDA and/or EMA approved anti-cancer treatment(s) e.g. one or more of chemotherapy, radiotherapy, immunotherapy, surgery, immuno-oncology, radioimmunotherapy, biological therapy, hormone therapy etc. In other words, in some embodiments, the combined anti-cancer effect of almitrine and another cancer treatment(s) is greater than the sum of each alone. In some embodiments, almitrine administration enables the same or greater anti-cancer activity to be exerted by another anti-cancer treatment(s) but with lower radiative (e.g. x-ray, y ray, electromagnetic radiation, radioactivity etc.) and/or drug(s) exposure, e.g. lower radio- and/or chemo-therapeutic(s) dose, most preferably enabling a lower side-effect profile. Encompassed herein is a method of reducing, treating and/or preventing adverse or undesired effects associated with conventional therapy including, but not limited to, chemotherapy, radiotherapy, immunotherapy, wherein almitrine or a pharmaceutically acceptable salt, solvate, hydrate, prodrug thereof, is administered to a subject prior to, during, or after the occurrence of the adverse effect associated with conventional therapy, optionally wherein the dosage/frequency/use of the conventional therapy is decreased. In some embodiments, almitrine is used in co-therapy with cisplatin and/or carboplatin and/or some other platinum based therapeutic(s) for anti-cancer treatment in a subject, and in further embodiments their anti-cancer activities synergize. In some embodiments, almitrine or a pharmaceutically-acceptable salt, solvate, hydrate or prodrug thereof is used in co-therapy with radiotherapy for anti-cancer treatment in a subject, and in further embodiments their anti-cancer activities synergize. In some embodiments, almitrine or a pharmaceutically-acceptable salt, solvate, hydrate or prodrug thereof is administered alongside radiotherapy to treat radioresistant cancer(s) and/or alongside chemotherapy to treat chemoresistant cancer(s).

Almitrine or a pharmaceutically-acceptable salt, solvate, hydrate or prodrug thereof can be administered exactly or approximately matching, or some function of, the pattern of administration of one or more of chemo- and/or radio-therapy given to the subject, wherein almitrine will add/synergize with their anti-cancer effects. To illustrate, a disclosure embodiment is to administer almitrine or a pharmaceutically-acceptable salt, solvate, hydrate or prodrug thereof (oral and/or injected) every day that radiotherapy is administered, wherein illustrative (not restrictive) courses of radiotherapy are to administer it every week day (one or more times) for a period that can vary between 3 to 9 weeks, or 3 times per day for 12 days (continuous hyperfractionated accelerated radiotherapy, CHART) or (especially with stereotactic radiotherapy) 3 to 8 times over 2 to 3 weeks, or accelerated fractionation, or hyperfractionation, or hypofractionation radiotherapy administration schemes. In further embodiments, almitrine or a pharmaceutically-acceptable salt, solvate, hydrate or prodrug thereof is also administered (oral and/or injected) at weekends during a weekday radiotherapy course, and/or during pauses in radiotherapy treatment and/or is administered (oral and/or injected) for a further period of administration flanking one or both sides of a radiotherapy treatment period. In some embodiments, almitrine or a pharmaceutically-acceptable salt, solvate, hydrate or prodrug thereof is administered during a period before and/or during and/or after anti-cancer surgery.

The i.v. dose of almitrine used in clinical practice at King's College Hospital (London, UK) is 8 μg/kg/min for COPD and 4-16 μg/kg/min for Acute Respiratory Distress Syndrome (ARDS) [156]. Intravenous, as opposed to oral, administration of almitrine or a pharmaceutically-acceptable salt, solvate, hydrate or prodrug thereof can be advantageous for anti-cancer therapy in a subject, especially in clinical trialing, because subject variability in oral bioavailability of almitrine is rendered irrelevant. An aspect to this is that, unlike oral administration, i.v. dosing, if sufficiently large, permits one to know precisely when peak plasma [almitrine] occurs in each subject. It being at the final time point of i.v. administration. This is useful when almitrine or a pharmaceutically-acceptable salt, solvate, hydrate or prodrug thereof is administered with radio- and/or chemo-therapy and maximal therapeutic synergy is desired. For example, if radiotherapy can't be administered during, it should be administered as soon as possible after, the i.v. administration of almitrine or a pharmaceutically-acceptable salt, solvate, hydrate or prodrug thereof. The shorter the delay between the end of i.v. almitrine administration and the start of radiotherapy, the more preferred the embodiment. Most preferred is the delay is zero and they are concurrent. During concurrent administration, radiotherapy preferably starts after the start of i.v. almitrine administration, to permit a build up of [almitrine] in the tissues before radiotherapy commences.

In humans, infused ˜7.47 ug/kg/min for 2 hours, delivering 60 mg almitrine dimesylate, wherein the mean almitrine plasma level in the subjects was 327 ng/ml at the end of the infusion period (lowest observed=242 ng/ml), which then fell to 157, 154, 105, 67, 55 ng/ml, at 15, 30, 60, 120, 600 minutes afterwards respectively. In humans, infused 8 μg/kg/min for 20 minutes during which the mean plasma concentration of almitrine was 325 ng/ml. In humans, [176] infused 8.3 μg/kg/min for 30 minutes, wherein “no adverse side-effect was observed during or after the administration of almitrine”. In humans, [177] infused 16 μg/kg/min for 1 hour, during which plasma almitrine concentration rose to be greater than 600 ng/ml in all subjects, much greater in some subjects (˜1,600 ng/ml highest observed), and by 2 hours after the infusion stopped, this had fallen to below 400 ng/ml in all subjects but one, and by 12 hours was below 200 ng/ml in all. In humans, infused 16.7 ug/kg/min for 1 hour “without deleterious effects”. In humans, infused 16 μg/kg/min and 20 minutes into this infusion the mean plasma concentration of almitrine was 659 ng/ml, wherein “plasma lactate concentrations remained within the normal range in all patients”. There are many more human studies in the literature wherein ˜16 or 16 μg/kg/min is infused, and many more wherein less is infused. Someone of the art will know how to find all these. A fraction, but by no means all, are listed and usefully compared in a table in [157]. In humans, infuses a higher rate than ˜16 μg/kg/min, =25 μg/kg/min for 1 hour. [157], a French regulatory review document for injectable almitrine, states “maximum recommended flow-rate: 15 mg/minute”, which corresponds to a flow rate of 242 μg/kg/min for a 62 kg human. In humans, gave a rapid intravenous (bolus) injection of 0.5 mg/kg, followed by infusing 2 mg/kg (corresponds to 16.7 μg/kg/min for a 62 kg human) for 2 hours, giving 2.5 mg/kg (corresponds to 155 mg for a 62 kg human) in just ˜2 hours. 200 mg (≙3.23 mg/kg for a 62 kg human) almitrine dimesylate orally per day has clinical precedent [259], as does an i.v. infusion rate of 16 μg/kg/min [156]. Combining these, one arrives at a daily i.v. infusion of 16 μg/kg/min for 202 minutes (3 hours and 22 minutes) to deliver 200 mg to a 62 kg human. Because, on average across subjects, only 85% of the oral dose is bioavailable, this oral dose can actually be approximated (in a 62 kg human) by a daily i.v. infusion of 16 μg/kg/min for 171 minutes (2 hours and 51 minutes). To illustrate, and not restrict, to deliver this same dose (to a 62 kg human) of almitrine dimesylate by continuous i.v., with an infusion rate of 8, 32, 64 μg/kg/min would take 5.7, 1.43 and 0.71 hours respectively. Optionally, infusion time can be shortened by injecting a proportion of the daily dose as i.v. bolus, optionally preceding the steady infusion. So, to illustrate, if 15 mg was administered by a preceding i.v. bolus (permissible by “maximum recommended flow-rate” instruction of [157]) then the duration required (in a 62 kg human) to replicate a 200 mg oral dose (factoring in bioavailability issue) would be 159 minutes of 16 μg/kg/min continuous i.v. infusion. In some disclosure embodiments, almitrine or a pharmaceutically-acceptable salt, solvate, hydrate or prodrug thereof is administered orally and by i.v. in combination to treat/ameliorate/prevent/combat cancer in a subject. So, to illustrate (not restrict), per day, 100 mg is administered orally and 100 mg is administered intravenously by 16 μg/kg/min for (in a 62 kg human) 101 minutes, 86 minutes if 15 mg of the i.v. dose is given by bolus. Optionally, the oral and i.v. administrations are timed so that as the i.v. administration finishes, and as plasma [almitrine] from the i.v. dose declines, almitrine from the oral dose enters the bloodstream to buoy/increase plasma [almitrine]. FIG. 3.3. in shows (data normalised to a 1 mg/kg dose) that the gradient of increase in plasma [almitrine] in the first 3 hours after an oral almitrine dose is similar to the gradient of decrease in plasma [almitrine] in the first 3 hours after an i.v. dose of almitrine terminates. So, administering oral almitrine as i.v. almitrine administration finishes can act to buoy/increase plasma [almitrine] for a longer period of time. This is very useful if the subject is to undergo radio- and/or chemo-therapy afterwards, wherein a high plasma [almitrine] is desired to gain the maximal additive/synergistic anti-cancer effect. All aforementioned dosages, routes/patterns of administration and infusion rates of almitrine or a pharmaceutically-acceptable salt, solvate, hydrate or prodrug thereof, in use for anti-cancer therapy/treatment in a subject, are componentry to the present disclosure. As are others, which are also contemplated by the scope of this disclosure. For example, the administered daily dosage could be greater than 200 mg. Indeed, relevantly, 400 mg oral almitrine dimesylate per day has been shown safe in healthy subjects, at least for the duration of that study [163]. Or the administered dosage could be less.

Intravenous infusion of 5.5 (±1.7) ug/kg/min almitrine dimesylate, in co-therapy with inhalation of nitric oxide (5 p.p.m.), increased arterial pO₂ by >30% across all subjects [183]. There are many similar papers in the literature, describing co-administration of almitrine and NO, which can be found readily by someone of the art. In some disclosure embodiments, almitrine or a pharmaceutically-acceptable salt, solvate, hydrate or prodrug thereof is administered, optionally intravenously, in co-therapy with nitric oxide (NO), optionally breathed, to treat/ameliorate/prevent/combat cancer in a subject, optionally in co-therapy with radio- and/or chemo-therapy, wherein the almitine and NO combination increases the subject's blood and tissue pO₂, which makes radio- and/or chemo-therapy more effective, adding to/synergising with the inherent anti-cancer activity of almitrine. At any point that almitrine administration is referred to in this disclosure, in further embodiments of this disclosure, NO is administered also.

Componentry to this disclosure is administering an i.v. bolus dose of almitrine or a pharmaceutically-acceptable salt, solvate, hydrate or prodrug thereof for anti-cancer therapy in a subject, optionally wherein the subject has lung cancer, and optionally wherein the almitrine concentration in the body is subsequently increased/prolonged by a subsequent period(s) of continuous i.v. infusion (optionally where the infusion rate equals/approximates the almitrine elimination rate) and/or one or more oral administrations of almitrine or a pharmaceutically-acceptable salt, solvate, hydrate or prodrug thereof (non-limiting e.g. tablet and/or solution). Optionally, wherein a foundational concentration of almitrine in subject plasma has been built up, prior to the i.v. dose of almitrine or a pharmaceutically-acceptable salt, solvate, hydrate or prodrug thereof, by one or more oral and/or i.v. doses of almitrine or a pharmaceutically-acceptable salt, solvate, hydrate or prodrug thereof, optionally administered on the same day and/or at some regular/irregular frequency, e.g. daily, in the day(s)/week(s)/month(s) before. Faster almitrine build up in the body can be achieved by dividing the daily dose up into multiple smaller doses, e.g. (non-limiting)₂₀₀ mg per day is administered by 100 mg administered twice per day, which means the build up period can be shorter.

At every point that radiotherapy is referred to in this disclosure, it encompasses all that a person of the art would expect it to. Including, without limitation, external (including, without limitation, one or more of external beam radiation therapy {e.g. using photons/electrons/hadrons/protons/neutrons/ions/nuclei etc.}, stereotactic body radiation therapy [SBRT], radiosurgery, 3-dimensional conformal radiation therapy, image-guided radiation therapy, intensity-modulated radiation therapy, tomotherapy, volumetric modulated arc therapy, particle therapy, proton therapy, neutron capture therapy, auger therapy) and/or internal (including, without limitation, one or more of brachytherapy, unsealed source radiotherapy, intraoperative radiotherapy, deep inspiration breath-hold, selective internal radiation therapy) radiotherapy. Moreover, when radiotherapy is referred to in this disclosure it encompasses radiotherapy with or without the co-administration of excess oxygen, wherein the subject breathes gas with a greater O₂ fraction than normal air at that altitude, optionally pure O₂, optionally the subject is administered hyperbaric O₂ therapy. An embodiment of this disclosure is to administer almitrine or a pharmaceutically acceptable salt, solvate, hydrate, prodrug thereof to a subject with cancer, optionally lung cancer, either orally and/or by i.v. (bolus and/or continuous) and/or by some other administration route, prior (on the same day and/or on prior day [s]) and/or during and/or after radiotherapy and/or chemotherapy. In some embodiments, almitrine or a pharmaceutically acceptable salt, solvate, hydrate, prodrug thereof is i.v. administered (bolus and/or continuous) before, during and after the subject undergoes radiotherapy and/or chemotherapy (optionally administered by i.v. also, bolus and/or continuous, in a separate or same infusion line to the subject), alternatively only before, or only during or only after, alternatively only before and after, alternatively only before and during, alternatively only during and after. In some embodiments, the administered almitrine enables a lower radiative/ionizing/chemotherapeutic(s) dose to be used to convey therapy. Optionally, it permits the same radiative dose to be used but over a longer timeframe, so the radiative intensity (per unit time) is less. Alternatively, the same radio- and/or chemo-therapy dose is used and greater therapeutic effect ensues. In a further embodiment, the subject has cancer and in more particular embodiments, the subject has lung cancer. In some disclosure embodiments a high ug/kg/min i.v. infusion rate, e.g. ≥8 μg/kg/min or ≥16 μg/kg/min or ≥64 μg/kg/min, of almitrine or a pharmaceutically acceptable salt, solvate, hydrate, prodrug thereof is delivered to a subject, optionally that has cancer, optionally lung cancer, prior and/or during and/or after radiotherapy and/or chemotherapy. A high infusion rate (by bolus only, or bolus+continuous, or continuous only) optimizes for all, or a significant proportion of, the daily almitrine dose, which in an embodiment is 200 mg but in other embodiments is higher or lower, being inside the subject at or around the time that radiotherapy and/or chemotherapy is administered, which gives the best opportunity for therapeutic synergy to occur. In some embodiments, i.v. (bolus and/or continuous) almitrine or a pharmaceutically acceptable salt, solvate, hydrate, prodrug thereof is administered preceding radio- and/or chemo-therapy and when this i.v. administration is stopped an oral dose of almitrine or a pharmaceutically acceptable salt, solvate, hydrate, prodrug thereof is administered so that the amount of almitrine entering the bloodstream from the oral dose partially/completely/exceeds the amount of i.v. dose almitrine eliminated from the body, which buoys the plasma almitrine concentration, which gives greater opportunity for anti-cancer therapeutic synergy between almitrine and radio- and/or chemo-therapy. In some embodiments, on days that the subject is administered radio- and/or chemo-therapy, all or some of the daily dose of almitrine or a pharmaceutically acceptable salt, solvate, hydrate, prodrug thereof is administered by i.v. (bolus and/or continuous), preferably near in time to the radio- and/or chemo-therapy, the nearer in time the better, most preferably coincident in time (for continuous i.v. infusion) or just before (for i.v. bolus only) or starting just before (for i.v. bolus followed my continuous i.v. infusion and/or oral administration), and, optionally, on days that the subject is not administered radio- and/or chemo-therapy, the daily dose of almitrine or a pharmaceutically acceptable salt, solvate, hydrate, prodrug thereof is absent or administered/taken orally, which the subject can do easily outside of a medical facility e.g. at home. So, for non-limiting example, during a daily course of radiotherapy and/or chemotherapy the subject receives almitrine or a pharmaceutically acceptable salt, solvate, hydrate, prodrug thereof by i.v. (bolus and/or continuous) and when this course of radiotherapy and/or chemotherapy pauses or finishes, and/or before it starts, the subject receives almitrine or a pharmaceutically acceptable salt, solvate, hydrate, prodrug thereof, optionally daily, orally, for a duration decided by a medical practitioner(s) and/or the subject.

For i.v. administering almitrine or a pharmaceutically acceptable salt, solvate, hydrate, prodrug thereof, and/or one or more chemotherapeutics (e.g. cisplatin, carboplatin etc.), during radiotherapy an uncharacteristically long i.v tube is preferable to keep the bulk of the i.v. equipment further away from the radiotherapy beam, most preferably wherein it is located behind some shielding. Optionally its radioactivity, or lack thereof, is regularly checked (e.g. using a Geiger counter and/or using some radioactivity sensor that stays on the equipment always {e.g. similar to that worn by workers that work in radioactive risk settings e.g. a dosimeter}), in an embodiment, before each use, and if too high, it is replaced with new equipment. In some embodiments, radioactive/electromagnetic/ionizing shielding is incorporated into the equipment itself. Preferably, the i.v. administration site(s) upon the subject shouldn't be directly under the radiotherapy beam and should be appropriately shielded as much as possible. In some embodiments, multiple i.v. lines are incident upon the subject undergoing radiotherapy, optionally wherein different chemotherapeutics are adminstered by the different lines, optionally wherein one or more i.v. lines administer almitrine or a pharmaceutically acceptable salt, solvate, hydrate, prodrug thereof.

In some embodiments, almitrine or a pharmaceutically acceptable salt, solvate, hydrate, prodrug thereof is i.v. administered (bolus and/or continuous), optionally in the same i.v, infusion, with one or more chemotherapies to a subject with cancer, optionally a lung cancer patient. In some embodiments, the timing of administering almitrine or a pharmaceutically acceptable salt, solvate, hydrate, prodrug thereof (e.g. oral or i.v. [bolus and/or continuous]), and the timing of administering one or more chemotherapies (e.g. oral or i.v. [bolus and/or continuous]), is coordinated so that peak plasma almitrine concentration occurs at the same time as the peak plasma concentration of chemotherapeutic(s), whereupon optionally, at this peak time, radiotherapy is administered.

In some embodiments, almitrine or a pharmaceutically acceptable salt, solvate, hydrate, prodrug thereof is orally administered before and/or during and/or after the subject, who in a further embodiment has cancer, optionally lung cancer, undergoes radiotherapy and/or chemotherapy; most preferably this oral almitrine is administrated before radiotherapy and/or chemotherapy and in further embodiments the timing of this oral almitrine administration vs. timing of radiotherapy and/or chemotherapy is co-ordinated so that the peak plasma concentration of almitrine occurs during or near in time to the radiotherapy and/or chemotherapy (N.B. in human, peak plasma concentration of almitrine occurs 3.5±0.7 hours after consuming almitrine dimesylate, absorption is improved by eating food [151]). In further embodiments, an oral course (e.g. daily, or other frequency) of almitrine administration, or a pharmaceutically acceptable salt, solvate, hydrate, prodrug thereof, is started days/weeks/months before a course of radiotherapy and/or chemotherapy starts, to build up a foundational level of almitrine inside the subject, and in further embodiments oral almitrine or a pharmaceutically acceptable salt, solvate, hydrate, prodrug thereof continues to be administered during the course of radiotherapy and/or chemotherapy and, in further embodiments, afterwards also. Before radio- and/or chemo-therapy, the number of days and the timing/frequency/dose of almitrine administered, or a pharmaceutically acceptable salt, solvate, hydrate, prodrug thereof, is optimized so that the plasma concentration of almitrine in the subject is suitably high (in some embodiments, to illustrate and not restrict, >300 ng/ml and/or >200 ng/ml) before radio- and/or chemo-therapy starts, wherein in a further embodiment the plasma concentration of almitrine in the subject is recorded to make sure. If not, the same or increased dose of almitrine or a pharmaceutically acceptable salt, solvate, hydrate, prodrug thereof is administered/taken to/by the subject, optionally more frequently, for a further time period before radio- and/or chemo-therapy commences.

In some disclosure embodiments, a therapeutically effective amount of almitrine or a pharmaceutically acceptable salt, solvate, hydrate, prodrug thereof, and/or an almitrine containing pharmaceutical composition, is administered to treat/ameliorate/prevent/combat cancer in a subject, optionally lung cancer, optionally Non-Small Cell Lung Cancer (NSCLC), optionally lung adenocarcinoma, optionally a PET positive cancer, optionally in co-therapy with radiotherapy, optionally wherein almitrine makes the cancer more radiosensitive/less radioresistant and/or the anti-cancer activities of almitrine and radiotherapy add/synergize, optionally in co-therapy with one or more chemotherapies, optionally wherein almitrine makes the cancer more chemosensitive/less chemoresistant and/or the anti-cancer activities of almitrine and chemotherap[y/ies] add/synergize, optionally in co-therapy with (without limitation) one or more of cisplatin, carboplatin, etoposide, gemcitabine, vincristine, vinblastine, paclitaxel, albumin-bound paclitaxel, vinorelbine, topotecan, irinotecan, temozolomide, docetaxel, pemetrexed, erlotinib, brigatinib, gefitinib, lorlatinib, afatinib, alectinib, denosumab, ipilimumab, pembrolizumab (Keytruda), nivolumab (Opdivo), atezolizumab (Tecentriq), durvalumab, dacomitinib, osimertinib, tivantinib, onartuzumab, sorafenib, crizotinib, ceritinib, trametinib, dabrafenib, bevacizumab (avastin), exisulind, bexarotene, cetuximab, squalamine, necitumumab, cyclophosphamide, ramucirumab, doxorubicin, porfimer, methotrexate, an FDA and/or EMA approved drug(s) or treatment(s) for lung cancer (including, without limitation, radiofrequency ablation, microwave ablation, cryoablation, thermal ablation, chemoembolization), optionally with plasma [almitrine] and/or [lactate] and/or [bilirubin] recording(s) before and/or during the course of almitrine administration, wherein if these are abnormally/too high, almitrine administration is reduced/stopped/not commenced. Almitrine greatly assists the treatment/amelioration/prevention/combat of NSCLC, which can be very radio (e.g. refer radioresistance of NCI-H460 cell line in [184]) and chemo-resistant, wherein almitrine undermines this resistance, enabling greater radio- and chemo-therapy to occur, improving the clinical outcome of the subject and/or their quality of life. It especially, without limitation, helps subjects with NSCLC that can't/won't undergo surgery, or whose cancer is inoperable, and who must completely rely on radio- and/or chemo-therapy for cancer treatment, wherein some NSCLC cancers are very radio- [184] and chemo-resistant and thence incredibly dangerous.

In some disclosure embodiments, (preferably a therapeutically effective amount of) almitrine or a pharmaceutically acceptable salt, solvate, hydrate, prodrug thereof, and/or an almitrine containing pharmaceutical composition, is administered to a subject topically/locally rather than systemically, optionally to a cancer(s) or close to a cancer(s) or to a blood vessel perfusing a cancer(s), wherein this cancer can be a tumour. In a particular embodiment, the cancer is suspected rather than diagnosed. In an embodiment, it is applied topically to the skin, optionally to a skin cancer(s) or suspected/possible skin cancer(s).

A disclosure embodiment is a pharmaceutical composition comprising a therapeutically effective amount of almitrine and a fatty acid(s), wherein the scope of what is a fatty acid is well known to those of the art. For non-limiting example, wherein almitrine and a fatty acid are in a 1:2 ratio. Other stoichiometries/ratios are also componentry to the present disclosure. For example, a 1:1 almitrine to fatty acid ratio. Some fatty acid(s) can exert anti-cancer activity (illustrative literature: [185-186]) and in preferred embodiments almitrine is combined in a composition(s) with a fatty acid(s) that exerts anti-cancer activity, and in more preferred embodiments the anti-cancer activity of almitrine and fatty acid(s) synergize. The greater the anti-cancer activity of the fatty acid(s) the more preferred the embodiment of its formulation with almitrine. In a further embodiment, an almitrine and fatty acid(s) containing composition(s) is used in a method of treatment of the human or animal body by therapy, for (non-limiting) example to treat/ameliorate/prevent/combat cancer in a subject. In another embodiment, an almitrine and fatty acid(s) containing composition is used for the manufacture of a medicament for the treatment/amelioration/prevention/combat of cancer. A disclosure embodiment is almitrine in a 1:2 stoichiometry with 9Z,11E conjugated linoleic acid (Rumenic Acid), which is an example of a fatty acid with anti-cancer activity [186]. In other illustrative example embodiments almitrine is in a 1:2 stoichiometry with Eicosapentaenoic Acid, or Docosahexaenoic Acid, or Erucic acid. Example embodiment:

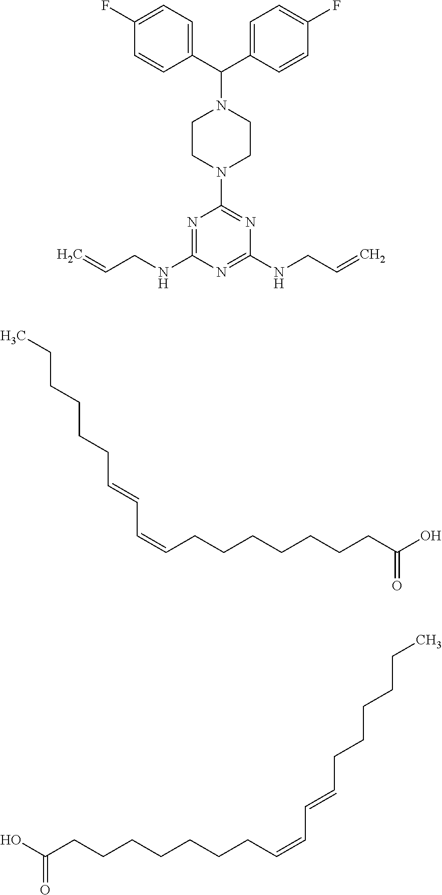

### EXAMPLE (VII)

### Formula (VII):

Compounds of Formula (VII) include any proteinaceous compound/amino acid sequence/peptide/protein/polypeptide/antibody that preferentially/disproportionally/selectively inhibits the “reverse”, ATP hydrolysing, mode as compared to the “forward”, ATP synthesising, mode of ATP synthase;

A polynucleotide that codes for at least one peptide/protein sequence of Formula (VII) is also a compound of Formula (VII);

A vector/gene therapy comprising at least one polynucleotide that codes for at least one peptide/protein sequence of Formula (VII) is also a compound of Formula (VII).

Note that selectively inhibiting F₁F₀ ATP hydrolysis can seemingly inhibit F₁F₀ ATP synthesis because less ATP is made, but this is because less ATP is hydrolysed and so less ATP needs to be made, rather than any actual direct inhibition upon F₁F₀ ATP synthesis. Preferred embodiments are those that inhibit the reverse mode of ATP synthase potently, and the forward mode of ATP synthase less potently, and most preferably not at all. If F₁F₀ ATP synthesis falls because of inhibited F₁F₀ ATP hydrolysis, and not primarily because of direct inhibition of F₁F₀ ATP synthesis, then this compound is still within the scope of this disclosure.

Componentry to Formula (VII) is melittin, the pre-sequence of yeast cytochrome oxidase subunit IV and each synthetic derivative of this pre-sequence ([4], incorporated in its entirety). Also componentry to Formula (VII) is one or more IF1 proteins, which are afforded their own Formula below: Formula (VIII). These examples just given for Formula (VII), and further examples herein, are illustrative and not restrictive.

Componentry to this disclosure is:
a pharmaceutical/cosmetic composition/medicament comprising (or consisting of) at least one compound of Formula (VII) and/or a pharmaceutically/cosmetically acceptable salt, solvate, hydrate, prodrug, liposome, nanoparticle (e.g. lipid nanoparticle, LNP) or other vector of the art thereof;
at least one polynucleotide of Formula (VII) in at least one vector/gene therapy of the art [and/or a cell(s)/transgenic organism(s) thereof] and a pharmaceutical/cosmetic composition/medicament thereof.

### EXAMPLE (VIII)

### Formula (VIII):

Compounds of Formula (VIII) include (from any organism, preferably a eukaryote) any IF1 protein, and any (preferably functional) sequence variant thereof, and any (preferably functional) amino acid sub-sequence/fragment of an IF1 protein, and any (preferably functional) sequence variant thereof, and any fusion protein thereof, wherein “functional” in this sentence refers to an ability to inhibit/reduce F₁F₀ ATP hydrolysis (e.g. inside a cell, preferably a eukaryote cell, and/or in a Sub-Mitochondrial Particle [SMP] assay of F₁F₀ ATP hydrolysis [functional in such an SMP assay with endogenous/native IF1 protein removed and/or present]);
Compounds of Formula (VIII) include the peptides/proteins of the teaching of this disclosure (e.g. sequences within this disclosure), for example those comprising (or consisting of) a sequence found within its Sequence Listing, for example SEQ ID NO:X, wherein X can be 1, or the number of sequences in the Sequence Listing component to this application, or any integer between 1 and the total number of sequences in the Sequence Listing component to this application;
   wherein “functional” (as defined above) sequence variant of SEQ ID NO:X, concatenated sequences of SEQ ID NO:X (or “functional” sequence variant thereof), fragment of SEQ ID NO:X (or “functional” sequence variant thereof), concatenated fragments of one or more of SEQ ID NO:X (or “functional” sequence variant thereof) are also included;
A polynucleotide that codes for at least one peptide/protein sequence of Formula (VIII) is also a compound of Formula (VIII);
A vector/gene therapy of the art comprising at least one polynucleotide that codes for at least one peptide/protein sequence of Formula (VIII) is also a compound of Formula (VIII).

A pharmaceutical/cosmetic composition/medicament comprising (or consisting of) at least one compound of Formula (VIII) and/or a pharmaceutically/cosmetically acceptable salt, solvate, hydrate, prodrug, liposome, nanoparticle (e.g. lipid nanoparticle, LNP) or other vector of the art thereof;
at least one polynucleotide of Formula (VIII) in at least one vector/gene therapy of the art [and/or a cell(s)/transgenic organism(s) thereof] and a pharmaceutical/cosmetic composition/medicament thereof.

Larger species tend to have more IF1 protein per unit mass, and/or use an IF1 protein that has greater inhibitory potency for F₁F₀ ATP hydrolysis, a lower specific metabolic rate, less metabolic heat production per unit mass, and longer lifespan (FIG. 4). Increasing the amount of at least one IF1 protein, and/or expressing at least one IF1 protein/fragment (or sequence variant thereof) that has (preferably greater) inhibitory potency for F₁F₀ ATP hydrolysis at pH 8, decreases metabolic rate and extends lifespan, so long as exogenous heat substitutes for the lesser endogenous heat production, or greater body insulation (e.g. wearing more clothes) renders same body temperature for a lessor metabolic rate. Componentry to this disclosure is extending the lifespan and/or healthspan of a subject, for example a mouse or human, by increasing the amount of the subject's own IF1 protein sequence and/or by introducing one or more exogenous IF1 protein sequences (and/or sequence variant(s) thereof, and/or fragment and/or concatenated fragments thereof [and/or sequence variant(s) thereof]), optionally by introducing at least one polynucleotide sequence(s) that codes for at least one IF1 protein/fragment (or sequence variant thereof), from a larger and/or longer living (greater maximal lifespan) species. The naked mole rat (Heterocephalus glaber) expresses ATPIF1 five times more than the mouse [187], and lives ten times longer. An embodiment is to express the IF1 gene (National Center for Biotechnology Information [NCBI] Gene ID: 101712500), and/or its further IF1 like gene (NCBI Gene ID: 110349814), of the naked mole rat in a homeothermic species (e.g. mouse/rat) to slow its metabolism and extend its lifespan, with the caveat that this species will then acquire some/all of the thermoconformer character of the naked mole rat and so must be kept at a higher ambient temperature. Naked mole rats are thermoconformers, which can survive without homeothermy because they live in hot East African countries, permanently underground in burrows (wherein heat is maintained at night), even eating underground (tubers), and can behaviourally thermoregulate by choosing their depth in the burrow.

IF1 proteins are highly conserved and typically interchangeable between species [141]. An embodiment is to increase the amount of IF1 protein in one or more cells of a subject and/or to administer/express in the subject at least one IF1 protein(s) from a different species (preferably a larger species and/or species with greater maximal lifespan) and/or at least one IF1 protein fragment and/or at least one IF1 protein/fragment sequence variant, most preferably which has greater inhibitory action against F₁F₀ ATP hydrolysis at normal matrix pH [˜8] (e.g. bovine/human IF1 protein with a H49K substitution (“mature” {Mitochondrial Import Sequence [MIS] cleaved off} IF1 protein numbering) [141, 138]). Wherein methods of introducing one or more of one or more genes and/or gene copies and/or DNA and/or RNA and/or one or more proteins into a subject are well known to those of the art e.g. refer [130], which expresses in mice the human IF1 protein (with H49K substitution, which increases its inhibition of F₁F₀ ATP hydrolysis at normal matrix pH [˜8]). An embodiment is expression in a subject of a bovine IF1 protein variant, which has one or more of its histidine residues at positions His-48, 49, 55 independently changed to a different amino acid, optionally alanine or lysine. In different embodiments, IF1 protein variant from a non-bovine species is expressed in a subject, wherein this IF1 protein is changed at one or more of the equivalent histidine positions to those aforementioned for the bovine IF1 protein sequence (these histidines are highly conserved across species, FIG. 10), which renders the IF1 protein with greater inhibitory potency against F₁F₀ ATP hydrolysis at normal matrix pH [˜8] [141, 138].

The use of one or more IF1 proteins, or a precursor thereof (e.g. with Mitochondrial Import Sequence (MIS) bound), which has its “phosphorylation control switch” amino acid residue and/or one or more of its “pH dependence motif” amino acid residues (FIG. 10) independently changed to be a different amino acid than found in the native IF1 protein(s), (and/or at least one polynucleotide that codes for one or more of the aforementioned, optionally with at least one gene expression control element(s), optionally a vector(s) thereof) in/for the manufacture of a medicament and/or pharmaceutical/cosmetic composition. Wherein to illustrate, using human “mature” (after Mitochondrial Import Sequence (MIS) cleaved) IF1 protein numbering (but not restricting to human IF1 protein): the “phosphorylation control switch” amino acid residue is S14 and the pH dependence motif amino acid residues are E26, H48, H49, H55, H56. To illustrate (not restrict), S14 is substituted with a different amino acid, which cannot be phosphorylated, illustratively alanine, and one/or one or more of E26, H48, H49, H55, H56 are independently substituted with another amino acid, illustratively (but not restrictively) selected from alanine, lysine, arginine. Especially preferred is S14A and H49K (or H49A or H49R), optionally modifying at one or more of the 4 other positions (e.g. substituting to alanine) also.

Contemplated by this disclosure, without limitation, is an organism IF1 protein, plant IF1 protein, animal IF1 protein, mammalian IF1 protein, mouse IF1 protein, rat IF1 protein, rodent IF1 protein, naked mole-rat IF1 protein, rabbit IF1 protein, guinea pig IF1 protein, bovine IF1 protein, canine IF1 protein, feline IF1 protein, pet/companion animal IF1 protein, livestock IF1 protein, equine IF1 protein, non-human primate IF1 protein and human IF1 protein.

All IF1 protein/fragment sequences, and sequence variants thereof, are componentry to this disclosure, as are the nucleotide sequences that code for them, and use thereof (for at least one use disclosed herein).

### Some IF1 Protein Fragment Embodiments

In IF1 protein depleted Sub-Mitochondrial Particles (SMPs) from Bos taurus, at pH 6.7 and 37° C., introduced Bos taurus IF1 protein (recombinantly produced in E. Coli) has an IC₅₀ of 0.034 μM [142]. In this same assay and study, Bos taurus IF1 protein fragments (using “mature” [without Mitochondrial Import Sequence, MIS] IF1 protein numbering) 14-84 and 10-47 have an IC₅₀ of 0.018 and 0.045 μM respectively, from which the authors of that study triangulate a “minimal inhibitory sequence” of residues 14-47. However, these fragments have the problem that their F₁F₀ ATPase inhibition dissipated over the 5 minute testing time: “ATPase activity deviates from linearity and increases with time”. The following IF1 protein fragments are longer, but are not reported to have this problem (IC₅₀ in μM in brackets): 10-84 (0.035), 1-56 (0.032), 1-60 (0.019), wherein the latter has a lower IC₅₀ than complete IF1 (showing that IF1 protein residues 61-84 are not essential for inhibitory activity at pH 6.7). Combining the teaching of these fragments, I disclose the following novel fragments, which have the benefit of being shorter (e.g. better for passaging across a biological membrane [s]): 10-56, 10-60. In [145], in a reportedly directly comparable assay to that used in (and indeed, they both report a similar IC₅₀ value for complete IF1 protein), IF1 protein fragment 42-58 has an IC₅₀ of 0.009 μM, which doesn't have the aforementioned problem of its inhibition decreasing in time. Fragments 14-47 and 42-58 have overlap with residues 42-47 (LAALKK [residues 29-34 of SEQ ID NO:661]), wherein this as a separate fragment, or as the IF1 protein fragment to use in a fusion protein of this disclosure (e.g. that comprises one or more of a Mitochondrial Import Sequence [MIS], Cell Penetrating Peptide [CPP], epitope/affinity tag sequence), optionally with one or more lipidic/lipid moieties (e.g. at least one fatty acid e.g. of between 2 and 25 carbon atoms) covalently bound, optionally incorporated into one cycle of a bicyclic structure, is an embodiment (optionally incorporated into a cosmetic/dermopharmaceutical). Presumably this core sequence is helped by an ancillary sequence for binding to ATP synthase, which can be on its C-terminal side (as in 42-58) or its N-terminal side, in which case a longer sequence is required (as in 14-47). As 42-58 can inhibit (0.009 μM) F₁F₀ ATP hydrolysis in [145], there is a mystery why 23-84, which contains 42-58, cannot (IC₅₀>100 μM) inhibit in [142]. Revealingly, 14-84 can inhibit (0.018 μM). So, it might be that residues 14-22 are required for the binding pathway, in order for key inhibitory resides to get to their correct place upon ATP synthase. But that 42-58 fragment is smaller and more nimble and binds by a different pathway, which doesn't require these N-terminal residues. Combining teaching of 42-58 (0.009 μM) and 1-56 (0.032 μM), I disclose 1-58 IF1 protein fragment, and with the teaching of 10-84 (0.035 μM), I disclose novel 10-58 IF1 protein fragment. Combining teaching of 42-58 (0.009 μM) and 1-56 (0.032 μM), I disclose novel 42-56 IF1 protein fragment. Herein, 14-47 is cited/used often as an exemplary IF1 protein fragment. Herein, at every point that a 14-47 IF1 protein fragment is referred to/shown, in alternative embodiments, a different IF1 protein fragment (optionally from same species/group of species as the context, or a different species/group of species as the context, or any species) is substituted in its place, for non-limiting example, selected from the group comprising: 42-58, 1-56, 1-60, 10-56, 10-60, 1-58, 10-58, 10-84 (or 42-56 or 42-47). In the case that a desired result isn't obtained with a 14-47 IF1 protein fragment, as this is probably a function of its (aforementioned) inhibitory effect decreasing in time, an alternative IF1 protein fragment, without this problem (as aforementioned, e.g. one of 42-58, 1-56, 1-60, 10-56, 10-60), should be utilized instead. But making due consideration that, although IF1 protein fragment 42-58 can potently inhibit bovine F₁F₀ ATP hydrolysis, this is not so much the case for rat ATP synthase (and so very possibly not mouse ATP synthase either). IF1 protein fragment 1-60 is a good, first option should 14-47 underperform in the system used. When an IF1 protein fragment (or sequence variant thereof) is referred to herein, in a different embodiment, a different IF1 protein fragment (or sequence variant thereof) is contemplated in its place, and across different embodiments, all possible IF1 protein fragments (or sequence variants thereof) are contemplated in its place. Contemplated IF1 protein fragments can differ for example in their length, wherein all possible lengths are contemplated: e.g. in different embodiments: shorter than (using “mature” [without MIS] IF1 protein numbering) z amino acids long, wherein z is an integer selected from 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 (different values of z are different embodiments). Some contemplated IF1 protein fragments include (using “mature” [without MIS] IF1 protein numbering) x-y, where x is an integer between 1 and 20 (or between 1 and 44, or between 1 and 84), and y is an integer between 40 and 85 (or between 50 and 85, or between 60 and 85, or between 2 and 85) [different values of x and/or y are different embodiments; within their aforementioned range constraints, all possible combinations of x and y integer values are contemplated]. Some contemplated IF1 protein fragments include 1-84, 2-84, 3-84, 4-84, 5-84, 6-84, 7-84, 8-84, 9-84, 10-84, 11-84, 12-84, 13-84, 14-84, 15-84, 16-84, 17-84, 18-84, 19-84, 20-84, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 81-84, 82-84, 83-84, and, for each aforementioned fragment, all possible sub-sequences/fragments thereof are also contemplated. Some non-limiting example IF1 protein fragments [wherein peptides/proteins comprising (or consisting of) one or more of these are contemplated] include 14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 10-84, 14-84, 14-60, 18-84, 10-50, 1-45, 42-56, 42-47, 48-56, 49-55.

### Some Novel IF1 Protein Fragment Embodiments

Using “mature” [without Mitochondrial Import Sequence, MIS] IF₁ protein numbering, with IC50 values (μM) in brackets, IF1 protein fragments 10-47 (0.045) or 1-60 (0.019) can inhibit F₁F₀ ATP hydrolysis in IF1 protein depleted bovine SMPs at pH 6.7, wherein the 1-60 fragment actually has a lower IC₅₀ than complete IF1 protein (0.034) [142]. So, residues more C-terminal than the 60^{th} residue aren't required to inhibit F₁F₀ ATP hydrolysis. And very arguably those more C-terminal to the 47^{th} residue. IF1 protein fragments 10-47 and 1-60 exist as monomers. The IF1 protein C-terminal region is involved in dimerization, tetramerization and higher oligomerization. IF1 protein monomers and dimers can inhibit F₁F₀ ATP hydrolysis, but dimer of dimers (tetramer) cannot because some or all the N-terminal inhibitory region (e.g. 14-47) is occluded in the dimer-dimer contacts. And nor can higher order oligomers. IF1 protein oligomerization state is pH dependent, wherein the C-terminal region confers this pH dependence (refer “pH dependence motif” in FIG. 10), tending to exist as active dimers at acidic pH and inactive tetramers (and higher oligomers) at alkaline pH (e.g. pH 8, the normal pH of mitochondrial matrix). By the teaching of this disclosure, this enables the therapeutic utility of a C-terminal IF1 protein fragment, which is short enough that it binds the C-terminal region of a complete IF1 protein without occluding its N-terminal inhibitory region, which blocks IF1 protein tetramerization/sequesteration/inactivation at alkaline pH, increasing the amount of active IF1 protein monomers/dimers at alkaline pH (e.g. pH 8, the normal pH of mitochondrial matrix). In some embodiments, this C-terminal fragment is from within the residue range (using “mature” [without MIS] IF1 protein numbering): residues: 61-85. In alternative embodiments, this C-terminal fragment is from within the residue range (using “mature” [without Mitochondrial Import Sequence, MIS] IF1 protein numbering): residues: 48-85, optionally comprising the H49 and/or H55 residues (importance of H49 and H55 to tetramerization has been experimentally shown), optionally comprising (or consisting of) part(s) or all of the HXXXXXH motif (within “pH dependence motif”, FIG. 10, residues 36-42 of SEQ ID NO:676), wherein X can be any amino acid coded for by the genetic code, optionally being, in different embodiments, shorter than c amino acids long, wherein c is an integer selected from 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 [different values of c are different embodiments], optionally being an IF1 protein fragment less than 20 amino acids long which comprises IF1 protein residues {using “mature” [without MIS] IF1 protein numbering}: 49-55, optionally comprising/consisting of residues: 48-56 and 1 to 10 (more preferably 1 to 5, optionally 1 to 3) surrounding (on either side) residues. Bovine IF1 protein fragment 44-84 exists as a dimer [189], and cannot form tetramers. Even at pH 8 [190]. Which suggests that there is at least one residue required for tetramerization more N-terminal than the 44^{th} residue. Indeed, triangulated to be in the region 32-44, because bovine IF1 protein fragment 32-84 can form tetramers [190]. But the aforementioned C-terminal occlusion strategy of this disclosure still works. Because, to block tetramerization, one has not got to occlude all the residues required for tetramerization, only a fraction of them. Most of which are in the C-terminal region of the IF1 protein (bovine IF1 protein fragment 1-60 doesn't form dimers, tetramers or higher oligomers [191, 143]). So, to repeat the approach: herein disclosed is an IF1 protein fragment (disproportionally/completely from the C-terminal region/half) that doesn't have the ability to inhibit F₁F₀ ATP hydrolysis directly itself. But that binds a C-terminal region of a complete IF1 protein, occluding its tetramerization domain, preventing its inactivation by tetramerization (and higher oligomerization) at alkaline pH, but not occluding its more N-terminal F₁F₀ ATP hydrolysis inhibiting domain, which increases the inhibition of F₁F₀ ATP hydrolysis at the normal/alkaline pH of mitochondrial matrix (pH 8). Advantageously, some examples of such an IF1 protein fragment can be shorter (better for intracellular delivery) than an IF1 protein/fragment that can inhibit F₁F₀ ATP hydrolysis directly itself. This approach relies upon complete IF1 protein being present. So these IF1 protein fragments will be observed to reduce F₁F₀ ATP hydrolysis, at pH 8, in an SMP assay of F₁F₀ ATP hydrolysis, when IF1 protein is present, and not (or very little) when IF1 protein amount is depleted beforehand. Preferred is to use SMPs sourced from big animal species, which tend to have more IF1 protein (e.g. bovine SMPs are favoured over those of rat), especially those reared in warm temperatures (tends to increase IF1 protein amount [117]).

### Different Mechanism of Action (MOA) and Consequently Different Species Preference

The species preference is different for different IF1 protein fragments. By the teaching of this disclosure, longer-living species tend to have more, and/or a more potent, IF1 protein (FIG. 4). To abstract somewhat, one can say that the N-terminal domain of an IF1 protein is for inhibiting F₁F₀ ATP hydrolysis, and its C-terminal domain is more for conferring its inactivation, via its tetramerization (and higher oligomerization), at the normally alkaline pH of the mitochondrial matrix (pH 8). To generalize further, the N-terminal domain of an IF1 protein from a longer-living species binds its ATP synthase more tightly/potently, and its C-terminal domain binds its other IF1 proteins {to form IF1 protein tetramers and higher oligomers} less tightly. The N-terminal domain of an IF1 protein from a shorter-living species binds its ATP synthase less tightly/potently and its C-terminal domain binds its other IF1 proteins {to form IF1 protein tetramers and higher oligomers} more tightly. So, a disproportionally N-terminal IF1 protein fragment (with most/all of its sequence residing in the N-terminal half of IF1 protein), which directly binds ATP synthase, and directly inhibits F₁F₀ ATP hydrolysis (e.g. the 14-47 fragment), is best sourced from a longer-living species (e.g. human, or blue or bowhead whale). But, because shorter-living species have more tightly bound IF1 tetramers {and higher oligomers}, a disproportionally C-terminal IF1 protein fragment (with most/all of its sequence residing in the C-terminal half of IF1 protein), which acts by binding another IF1 protein, is better sourced from a shorter-than a longer-living species. With the caveat that the IF1 protein of a species is tuned to its own ATP synthase. So, for example, although the N-terminal domain of an IF1 protein from a longer-living species binds its ATP synthase more tightly/potently, this doesn't necessarily mean, in all cases, that it can bind the ATP synthase of a shorter-living species more tightly/potently than the shorter-living species' own IF1 protein. Because of the evolutionary distance between the species. Wherein greater distance tends to decrease the opportunity for advantageous interoperability. Optionally an administered IF1 protein fragment is from a species not too far evolutionarily removed from a/each species to be administered. Optionally the disproportionally N-terminal or C-terminal IF1 protein fragment can be from the same species as that to be administered. Optionally the disproportionally C-terminal IF1 protein fragment can be from a short-living mammal, e.g. rodent, e.g. mouse.

The administration to a subject of a complete (or nearly complete) IF1 protein (optionally from a long-lived species e.g. human, or blue or bowhead whale), and a fragment from the C-terminal region of an IF1 protein (optionally from a shorter-lived species e.g. mouse), is contemplated, wherein this dual administration can confer an additive/synergistic effect.

### Summary of Two Different Mechanisms of Action

Without seeking restriction by theory, some IF1 protein fragments of this disclosure inhibit/reduce F₁F₀ ATP hydrolysis directly, by acting upon ATP synthase itself, and others bind the pH sensing region of complete endogenous IF1 protein and disrupt higher (≥tetramer) IF1 protein oligomerization, liberating IF1 protein dimers/monomers to inhibit/reduce F₁F₀ ATP hydrolysis. So, either directly or indirectly they reduce F₁F₀ ATP hydrolysis. One can discriminate between these two Mechanisms of Action (MOAs) by independently testing IF1 protein fragment in an SMP assay of F₁F₀ ATP hydrolysis, with and without endogenous/native IF1 protein removed (can remove IF1 protein, for example, via method of Horstman LL, Racker E 1970 J. Biol. Chem. 245, 1336-1344), wherein if it acts directly on ATP synthase it will inhibit in both cases, and if via IF1 protein, it will only inhibit in the first and not the second case.

### Some Preferred IF1 Proteins/Fragments (or Sequence Variants Thereof)

An IF1 protein fragment that still has enough residues that it can still inhibit F₁F₀ ATP hydrolysis, but which is without the IF1 protein's C-terminal pH dependent tetramerization (and higher oligomerization) domain (or at least not enough of it for it to be functional i.e. at least not enough of it that it can confer tetramer and higher oligomerization) has the advantage that it is constitutively active, i.e. it can constitutively inhibit F₁F₀ ATP hydrolysis, even at alkaline pH (e.g. at pH 8, which is the typical pH of mitochondrial matrix). Some IF1 protein fragments that fulfil this criteria include, but are not restricted to, residues (“mature” [without MIS] IF1 protein numbering): 1-60, 10-60, 14-60, 1-57, 10-57, 14-57, 10-47 and 14-47. An alternative to this C-terminal truncation is just to modify key residues within this C-terminal domain such that it can no longer confer pH dependent tetramerization (and higher oligomerization), e.g. refer to “pH dependence motif”, and modifications thereof (e.g. H49K or H49R substitution), in FIG. 10, to confer constitutive inhibition of F₁F₀ ATP hydrolysis, even at alkaline pH. An IF1 protein/fragment, or sequence variant thereof, that cannot (or doesn't have strong disposition to) form tetramers, and higher oligomers, which exists simply/disproportionally as IF1 protein/fragment (or sequence variant thereof) monomers and/or dimers, even at alkaline pH (e.g. pH 8), but which can still inhibit F₁F₀ ATP hydrolysis, is a preferred IF1 protein fragment. Even more preferred is such an IF1 protein/fragment (or sequence variant thereof) with its “phosphorylation control switch” residue [FIG. 10] being an amino acid that cannot be phosphorylated, optionally alanine.

### Further Preferred

Peptide/protein (optionally with one or more of its carboxyl groups esterified) comprising (or consisting of) [preferably wherein the following is in N to C-terminal order] at least one Cell Penetrating Peptide sequence (CPP, e.g. a poly-arginine CPP, optionally with a fatty acid [e.g. of between 2 to 25 carbons] acylated to its N-terminal end) conjoined with (e.g. peptide bonded to) at least one Mitochondrial Import Sequence (MIS; conferring mitochondrial matrix localization, optionally/preferably wherein the MIS is that used by the species administered to for its native IF1 protein; e.g. MIS that human uses for its native IF1 protein) conjoined with (e.g. peptide bonded to) at least one “mature” (without MIS) IF1 protein/fragment (or sequence variant thereof) that is optionally/preferably:
(a) an IF1 protein sufficiently truncated at its C-terminal end (non-limiting e.g. truncated up to {using “mature” [without MIS] IF1 protein numbering} its 60^{th} or 47^{th} residue), and/or optionally truncated at its N-terminal end (non-limiting e.g. by any number of residues up to 9 [or 13] residues), and/or with one or more amino acid substitutions in its “phosphorylation control switch” and/or “pH dependence motif” (FIG. 10; non-limiting e.g. one or more of {using “mature” [without MIS] IF1 protein numbering} S14A [or T14A], E26A [or Q26A or E26Q], H48A [or Y48A], H49K [or H49A or H49R], H55A [or Y55A or V55A], H56A [or T56A or S56A] substitutions), such that it can still inhibit F₁F₀ ATP hydrolysis but it cannot (or cannot as readily) form IF1 protein tetramers (and higher oligomers) at alkaline pH, preferably such that it can more potently inhibit F₁F₀ ATP hydrolysis at the normal, alkaline pH (˜pH 8) of the mitochondrial matrix (than native/unmodified IF1 protein), optionally/preferably wherein this IF1 protein/fragment (or sequence variant thereof) has a sequence derived/modified from the IF1 protein sequence of the species to be administered to [e.g. human] or a species with a longer maximal lifespan, optionally a species with a very long maximal lifespan such as a whale e.g. bowhead or blue whale; or
(b) an IF1 protein fragment comprising residues from the C-terminal half of an IF1 protein, which can bind to a complete IF1 protein, therein inhibiting or preventing (reducing the propensity for) complete IF1 proteins (e.g. monomers or dimers thereof) combining to form tetramers (or higher oligomers) thereof, but wherein its binding doesn't block complete IF1 protein monomers/dimers from inhibiting F₁F₀ ATP hydrolysis, preferably wherein it actually increases their inhibition of F₁F₀ ATP hydrolysis at the normal, alkaline pH of the mitochondrial matrix;
and use thereof (for at least one use disclosed herein, e.g. for treating cancer and/or slowing aging in a subject, e.g. as at least one component of a cosmetic), optionally wherein one or more amino acid sequences from both these general forms are co-administered to a subject, optionally/preferably wherein the fusion protein has an N- to C-terminal order: [CPP]-[MIS]-[IF1 protein/fragment (or sequence variant thereof)], optionally with an epitope/affinity tag conjoined at (e.g. peptide bonded to) the N-terminal end.

A molecule (e.g. small molecule or biologic) that inhibits or prevents (reduces the propensity for) IF1 proteins (e.g. monomers or dimers thereof) combining to form tetramers (or higher oligomers) thereof, but which doesn't block IF1 protein monomers/dimers from inhibiting F₁F₀ ATP hydrolysis, preferably wherein it actually increases their inhibition of F₁F₀ ATP hydrolysis at the normal, alkaline pH (˜pH 8) of the mitochondrial matrix; and use of this molecule (for at least one use disclosed herein, e.g. for treating cancer and/or slowing aging in a subject, e.g. as at least one component of a cosmetic).

If1 Protein Fragments from all Species are Contemplated

In some embodiments, the administered IF1 protein/fragment (or sequence variant thereof) is from the same species that the subject belongs to.

In some embodiments, the administered IF1 protein/fragment (or sequence variant thereof) is from a different species than the subject belongs to.

In some embodiments, the administered IF1 protein/fragment (or sequence variant thereof) is from a longer-living (higher maximal lifespan) species than the subject belongs to; wherein if the subject is a mammal, in some embodiments, the administered IF1 protein/fragment (or sequence variant thereof) is from a longer-living (higher maximal lifespan) mammal species.

In some embodiments, the administered IF1 protein fragment (or sequence variant thereof) is from a shorter-living (lower maximal lifespan) species than the subject belongs to; wherein if the subject is a mammal, in some embodiments, the administered IF1 protein fragment (or sequence variant thereof) is from a shorter-living (lower maximal lifespan) mammal species.

### Screening for Some IF1 Protein Fragment Embodiments

Method of screening for least one IF1 protein fragment that can inhibit/reduce F₁F₀ ATP hydrolysis in a Sub-Mitochondrial Particle [SMP] assay of F₁F₀ ATP hydrolysis, at alkaline pH (e.g. pH 8), in which endogenous/native IF1 protein is not removed.

This method will find IF1 protein fragments inhibiting F₁F₀ ATP hydrolysis by both the aforementioned mechanisms of action: those binding ATP synthase and inhibiting F₁F₀ ATP hydrolysis directly, and those binding a C-terminal region of complete endogenous IF1 protein, blocking its inactivation by tetramer (and higher oligomer) formation/sequesteration, but wherein its binding doesn't occlude the (predominantly)N-terminal inhibitory domain (e.g. 14-47 or 1-60 residues), thence freeing active IF1 protein dimers/monomers that can inhibit F₁F₀ ATP hydrolysis. If instead, IF1 protein depleted SMPs are used (e.g. via method of Horstman L L, Racker E 1970 J. Biol. Chem. 245, 1336-1344), only IF1 protein fragments working by the first mechanism will be found by this screening method. Because of this it is preferred that the SMPs are not IF1 protein depleted. And because this assay is performed at pH 8, and not pH 6.7, there is no need to remove the endogenous IF1 protein anyhow. Because most of it is inactivated by tetramerization (and higher oligomerization) at this alkaline pH.

A method of the above, wherein a number of different IF1 protein fragments are systematically tested, preferably wherein the first IF1 protein fragment tested consists of the most C-terminal (last) residue of an IF1 protein (non-limiting e.g. of Bos taurus), the second fragment tested consists of the last two residues, the third fragment consists of the last three residues, the fourth fragment consists of the last four residues, and this testing is iterated in this fashion, adding a residue each time (optionally testing until the N-terminal end of the IF1 protein is reached, or stopping before this, optionally stopping once the 47th residue [from the N-terminal end, using “mature” {without MIS} IF1 protein numbering] is reached, or when a residue nearby is reached). Then, with each fragment found to have inhibition/reduction of F₁F₀ ATP hydrolysis activity, the fragment sequence is tested again but with its most C-terminal (last) absent, and then again iteratively, each time with one more amino acid removed from its C-terminal end, until the activity is lost or until there are no residues remaining.

This method will find a number of different IF1 protein fragments that can inhibit/reduce F₁F₀ ATP hydrolysis at alkaline pH (e.g. pH 8). These are novel therapeutic peptides, which are componentry to this disclosure (as is their use, e.g. for at least one use disclosed herein), as are functional [e.g. verified by aforementioned SMP assay] sequence variants thereof (as is their use, e.g. for at least one use disclosed herein). And they are advantageously (e.g. for cellular entry alone or as part of a fusion protein) short.

Optionally wherein this method is repeated with fragments from the IF1 protein of a different species, optionally wherein it is performed with fragments from the IF1 protein of a number of different species.

In an optional next step(s), for each of the selected peptides, or for only one or more of the most potent (e.g. low EC₅₀ against F₁F₀ ATP hydrolysis), an alanine scan is performed, wherein each residue position is iteratively substituted to alanine, and the ability to inhibit/reduce F₁F₀ ATP hydrolysis at alkaline pH (e.g. pH 8), in an SMP assay of F₁F₀ ATP hydrolysis (with endogenous IF1 protein present and not removed), is assayed each time, to identify key amino acid residues, and, by contrast, those that can be changed without (much) loss of activity (or that actually increase activity), wherein sequence variants with an amino acid changed at one or more of these positions are componentry to this disclosure.

Also componentry are sequence variants wherein an amino acid(s) is changed to be a different amino acid that a different species has at the equivalent position in their IF1 protein.

Functional sequence variants of all these fragments are componentry to this disclosure (as is their use, e.g. for at least one use disclosed herein).

With these methods in hand, variations of these methods will be apparent to those of the art, which are herein contemplated, and componentry to this disclosure.

Optionally wherein each IF1 protein fragment(s) (or sequence variant(s) thereof) selected by this method (i.e. shown to reduce F₁F₀ ATP hydrolysis) is tested in an SMP assay of F₁F₀ ATP synthesis, optionally wherein if it appreciably reduces F₁F₀ ATP synthesis also, it is dismissed.

### ATP Synthase Protein Fragments are Contemplated

When IF1 protein binds ATP synthase, to inhibit its F₁F₀ ATP hydrolysis, the C-terminal domain of IF1 protein does still bind ATP synthase (although, as aforementioned, this binding isn't absolutely required for it to inhibit F₁F₀ ATP hydrolysis). Wherein, by some non-limiting reports, it may bind in the C-terminal region of the β sub-unit of F1. In some embodiments, a peptide/protein fragment comprising (or consisting of) a part(s) of ATP synthase that the C-terminal domain of IF1 protein binds, is used as a fragment to increase inhibition of F₁F₀ ATP hydrolysis at alkaline/normal pH of mitochondrial matrix (pH 8), wherein this fragment binds to the C-terminal domain of IF1 protein, preventing its inactivation via tetramerization (and higher oligomerization), freeing IF1 protein dimers/monomers that can inhibit F₁F₀ ATP hydrolysis.

### A Screening Methodology

A non-limiting method to find some amino acid sequence embodiments of this disclosure is to read the sequence of a known IF1 protein (or, in different embodiments, any amino acid sequence known to inhibit/reduce F₁F₀ ATP hydrolysis), or a fragment thereof, and to synthesize it, and/or sequence variants of this sequence which are within at least 30% or (in a different embodiment) at least 40% or (in a different embodiment) at least 50% or (in a different embodiment) at least 60% or (in a different embodiment) at least 70% or (in a different embodiment) at least 80% or (in a different embodiment) at least 85% or (in a different embodiment) at least 90% or (in a different embodiment) at least 95% sequence identity (and in further embodiments: >96%, >97%, >98%, >99%, >99.5%, >99.8% sequence identity; in some preferred embodiments >70% or >80% or >90% or >95% or 97% sequence identity is selected as a cut-off for sequences to test, above which a sequence is selected for testing, below which it isn't), and independently test each version in an SMP assay of the art (e.g. as described herein and/or one or more references herein), independently testing its action (or lack thereof) upon (firstly) F₁F₀ ATP hydrolysis and (if it reduces/inhibits F₁F₀ ATP hydrolysis) F₁F₀ ATP synthesis, to test whether it can specifically inhibit/reduce F₁F₀ ATP hydrolysis (i.e, wherein it doesn't inhibit/reduce F₁F₀ ATP synthesis at all, or only a small amount as compared to its inhibition/reduction of F₁F₀ ATP hydrolysis, in an SMP assay of the art; preferably testing for action upon F₁F₀ ATP hydrolysis first, and only if an effect is seen, is action upon F₁F₀ ATP synthesis assayed). If yes, it is an amino acid sequence embodiment of this disclosure, and its use (for at least one use disclosed herein) is hereby contemplated (optionally via incorporation into a fusion protein of this disclosure also comprising one or more of a CPP, MIS, epitope/affinity tag). Optionally, the SMP assay can be conducted at pH 6, in which case it is prudent to remove the endogenous IF1 protein in the assay beforehand (e.g. via method of Horstman L L, Racker E 1970 J. Biol. Chem. 245, 1336-1344), or optionally (and much more preferably) at pH 8, wherein amino acid sequences that can inhibit/reduce F₁F₀ ATP hydrolysis at pH 8 are especially favoured. Note that pH 8 is the typical pH of the mitochondrial matrix. When testing at pH 8, the endogenous IF1 protein can be removed beforehand, but this assay will then only report on amino acid sequences that can inhibit/reduce F₁F₀ ATP hydrolysis by acting directly on ATP synthase, and it will likely miss those amino acid sequences that inhibit/reduce F₁F₀ ATP hydrolysis indirectly by acting upon IF1 protein to break up higher (>dimer) IF1 protein oligomers (which form at alkali pH), liberating IF1 protein dimers/monomers that can then inhibit F₁F₀ ATP hydrolysis. To find the latter, using SMPs sourced from big animal species, which tend to have more IF1 protein [117], is preferred (e.g. bovine SMPs are favoured over those of rat), especially those reared in warm temperatures (tends to increase IF1 protein amount [117]). In a particular embodiment, if an amino acid sequence is already known to inhibit/reduce F₁F₀ ATP hydrolysis (for example if the method converges upon the entirety of a sequence of a known IF1 protein) then it is excluded from this method.

This method has been described with an SMP assay, but it can alternatively (or in addition) be used with an alternative assay of F₁F₀ ATP hydrolysis, for example in an applicable cellular/sub-cellular/molecular assay of the art.

In different embodiments, instead, or in addition, a different type of assay can be used, investigating whether the tested amino acid sequence can stop/reduce IF1 protein tetramer, and higher oligomer, formation at pH 8 (or higher), thence increasing the prevalence of the monomer and/or dimer IF1 protein species (e.g. using the assay or variant thereof of [190]). If it does, then preferably a 2^{nd} assay is performed, testing whether these IF1 protein monomers/dimers with the amino acid sequence bound can still inhibit F₁F₀ ATP hydrolysis, optionally by an SMP assay, preferably with its endogenous IF1 protein not removed (and preferably using SMPs sourced from a large animal species e.g. bovine), preferably performed at pH 8 (or higher).

When an amino acid sequence shorter than 150 amino acids long, and more preferably shorter than 100, and more preferably shorter than 50, and more preferably shorter than 20, and more preferably shorter than 10 amino acids long, and more preferably shorter than 7 amino acids long, is found to inhibit/reduce F₁F₀ ATP hydrolysis, in further embodiments it is (optionally with another amino acid [s], which can be non-proteingenic, e.g. a D-amino acid) partially/completely integrated into a cyclic form, preferably a bicyclic form, into both (not recommended because this might assist rather than disrupt IF1 protein tetramerization and higher oligomerization) or very preferably into just one of the cycles (if only one, then other cycle has random/CPP/MPP sequence and/or a sequence that can increase plasma stability and/or a sequence optimized for higher log P/lipophibicity and/or a CPP sequence with successful precedent use in a bicyclic form {e.g. refer U.S. Pat. No. 10,626,147B2} and/or other sequence), and is tested again in this SMP assay, in this form. Wherein if it still inhibits/reduces F₁F₀ ATP hydrolysis in this form, this form is then componentry to this disclosure and its use in this form (for at least one use disclosed herein) is hereby contemplated.

Administering to a subject a combination of peptide(s) and/or protein(s) and/or polynucleotide(s) and/or vector(s) and/or cell(s) and/or transgenic organism(s) of/in this disclosure is contemplated.

The (optionally synergistic) use (for at least one use disclosed herein) of a combination of more than one type of IF1 protein/fragment (or sequence variant thereof, or concatenation of fragments thereof) is componentry to this disclosure.

The (optionally synergistic) use (for at least one use disclosed herein) of a combination of at least one type of IF1 protein (or sequence variant thereof) and at least one type of IF1 protein fragment (or sequence variant thereof) is componentry to this disclosure. Synergy particularly applies when the IF1 protein fragment (or sequence variant thereof) acts to block/reduce higher (>dimer) oligomerization with the added IF1 protein (or sequence variant thereof). This is a preferred combination. Especially for use in a species that doesn't have much IF1 protein, which tends to be a smaller, faster heart rate, shorter-lived species such as mice (and so an IF1 fragment that blocks/reduces IF1 protein >tetramerization will work much better if complete IF1 protein is administered also). Nucleotide sequences encoding these combination of amino acid sequences are hereby contemplated. As are nucleotide sequences with gene expression control elements set to optimize the relative expression/stoichiometry of these amino acid sequences for most efficacious combination (or vector thereof), to inhibit/reduce F₁F₀ ATP hydrolysis, inside a cell.

Some Sequences of this Disclosure

Componentry to this disclosure is any peptide/protein/amino acid sequence, and/or use thereof (for at least one use disclosed herein), incorporated by the teaching of this disclosure, including any peptide/protein/amino acid sequence incorporated by the teaching of its Description section and/or actually shown/exemplified in its Description and/or Sequence Listing and/or Drawings.

Componentry to this disclosure is: a peptide/protein comprising (or consisting of) at least one amino acid sequence of/in this disclosure, a polynucleotide comprising (or consisting of) at least one nucleotide sequence of/in this disclosure. Herein, when an amino acid sequence is referred to, in preferred embodiments this refers to a peptide/protein comprising (or consisting of) this amino acid sequence. Herein, when a nucleotide sequence is referred to, in preferred embodiments this refers to a polynucleotide comprising (or consisting of) this nucleotide sequence.

Incorporated by reference is the material in the corresponding Sequence Listing to this application. Which is submitted as an ASCII text file, named “Sequence Listing”, created on 20 Jan. 2021, with a size of 3,594,615 bytes.

The corresponding Sequence Listing is componentry to this application. This Sequence Listing forms part of the international application.

“Sequence Listing Free Text”: text in one or more <223> entries (“Other information”) “which does not use language-neutral vocabulary as referred to in paragraph 2 (vii)” of WIPO Standard ST.25 (N.B. L-amino acids commonly found in naturally occurring proteins, in 3-letter code, are language neutral vocabulary and so are not shown here): “Xaa at this position is a single (one, 1) amino acid selected from the following options:”; “Xaa at this position is a single (one, 1) amino acid (or lack thereof) selected from the following options:”; “Xaa is a single (one, 1) amino acid (or lack thereof) INDEPENDENTLY selected for each of these positions from the following options:”; “or is absent (no amino acid at this position and so this position is not present)”; “If adjacent residues are both cysteine, optionally they can be connected by a disulfide, instead of a peptide, bond”; “Epitope/affinity tag”; “These amino acids are D-amino acids”; “Encompasses natural & artificial PRT sequences; artificial sequences designed by aligning (by their “pH dependence motif”) some natural IF1 protein sequences sourced from InterPro family IPR007648; more detail elsewhere in present application”; “Encompasses natural & artificial PRT sequences; artificial sequences designed by manual alignment (by their “pH dependence motif”) of natural IF1 protein sequences from some long-lived species; more detail elsewhere in present application”; “Encompasses natural & artificial PRT sequences; artificial sequences designed by manual alignment and manipulation (described elsewhere in present application) of some natural IF1 protein sequences”; “Designed; as detailed elsewhere in present application”; “Xaa is INDEPENDENTLY selected at each position from any amino acid or lack thereof (no amino acid at this position and so this position is not present)”; “OPTIONALLY wherein one or more amino acids have a (independently selected in each case) lipid moiety covalently attached e.g. fatty acid, cholesterol etc.”; “OPTIONALLY wherein there is a fatty acid acylated to this sequence's N-terminus”; “OPTIONALLY one or more lysine residues (if present) have a fatty acid conjugated/acylated to their side-chain”; “Cell Penetrating Peptide (CPP) sequence”; “Optionally wherein one or more of the amino-acids are corresponding D-amino acids”; “D-amino acid”; “INDEPENDENTLY selected at each position from: L- or D-Arg/Lys/Phe/Trp/Tyr/Gln/phenylglycine/diphenylalanine/cyclohexylalanine/3-2- or 3-1-naphthylalanine/aminooctanoic acid/O-Methyltyrosine/2,6-dimethyltyrosine or absent”; “INDEPENDENTLY selected at each position from: L- or D-Arg/Lys/Phe/Trp/diphenylalanine/cyclohexylalanine/3-2- or 3-1-naphthylalanine/aminooctanoic acid/2,6-dimethyltyrosine or absent”; “INDEPENDENTLY selected at each position from: L- or D-Arg/Phe/diphenylalanine/cyclohexylalanine/3-2- or 3-1-naphthylalanine/aminooctanoic acid/2,6-dimethyltyrosine or absent”; “INDEPENDENTLY selected at each position from: L- or D-Arg/Phe/diphenylalanine/cyclohexylalanine/3-2- or 3-1-naphthylalanine/2,6-dimethyltyrosine or absent”; “INDEPENDENTLY selected at each position from: L- or D-Arg/diphenylalanine/cyclohexylalanine/3-2- or 3-1-naphthylalanine/2,6-dimethyltyrosine or absent”; “INDEPENDENTLY selected at each position from: L- or D-Arg/diphenylalanine/3-2- or 3-1-naphthylalanine/2,6-dimethyltyrosine or absent”; “INDEPENDENTLY selected at each position from: L-arginine or D-arginine or 2,6-dimethyl-L-tyrosine or 2,6-dimethyl-D-tyrosine”; “INDEPENDENTLY selected at each position from: L- or D-Arg/3-2- or 3-1-naphthylalanine, 3,3-diphenyl-L-alanine, 3,3-diphenyl-D-alanine, or absent”; “INDEPENDENTLY selected at each position from: L-arginine, D-arginine, L-phenylalanine, D-phenylalanine or absent”; “INDEPENDENTLY selected at each position from: L- or D-diphenylalanine/cyclohexylalanine/3-2- or 3-1-naphthylalanine/2-aminooctanoic acid/2,6-dimethyltyrosine or absent”; “INDEPENDENTLY selected at each position from: L-arginine or D-arginine or absent”; “L- or D-diphenylalanine/cyclohexylalanine/3-2- or 3-1-naphthylalanine/2-aminooctanoic acid/2,6-dimethyltyrosine or absent L-arginine or D-arginine or absent”; “D-arginine”; “L-3-cyclohexylalanine or D-3-cyclohexylalanine”; “L-3-(1-naphthyl) alanine or D-3-(1-naphthyl) alanine or L-3-(2-naphthyl) alanine or D-3-(2-naphthyl) alanine”; “L-2-aminooctanoic acid or D-2-aminooctanoic acid”; “3,3-diphenyl-L-alanine or 3,3-diphenyl-D-alanine”; “O-Methyl-L-tyrosine or O-Methyl-D-tyrosine”; “D-phenylalanine”; “L-phenylglycine or D-phenylglycine”; “any amino acid or absent”; “D-glutamine”; “L-3-cyclohexylalanine or D-3-cyclohexylalanine”; “2,6-dimethyl-L-tyrosine or 2,6-dimethyl-D-tyrosine”; “L- or D-diphenylalanine/cyclohexylalanine/3-2- or 3-1-naphthylalanine/2-aminooctanoic acid/2,6-dimethyltyrosine”; “L- or D-diphenylalanine/cyclohexylalanine/3-2- or 3-1-naphthylalanine/2-aminooctanoic acid/2,6-dimethyltyrosine or absent”; “L-arginine or D-arginine”; “L-arginine or D-arginine or absent”; “L-phenylalanine or D-phenylalanine”; “L-phenylalanine or D-phenylalanine or absent”; “Xaa can be any naturally occurring amino acid”; “This Cys is disulfide bonded to the Cys in the following sequence:”; “Synthetic fragment”.

In the Sequence Listing component of this application, at one or more locations where <221> (Name/key of feature) is populated with “MISC_FEATURE” or “misc_feature”, in alternative embodiments this is populated with “VARIANT” instead.

The WIPO ST.25 standard doesn't permit the showing of a D-amino acid(s). For all sequences in the Sequence Listing of this application, in other disclosure embodiments, a corresponding D-amino acid replaces an L-amino acid at one or more places (e.g. D-arginine replaces L-arginine at one or more places).

At some points herein, an IF1 protein is referred to as “mature”, when it is without its Mitochondrial Import Sequence (MIS) attached, and “immature” when an MIS is attached. By default (unless the context suggests otherwise), at every point that an IF1 protein/fragment (or sequence variant thereof) is referred to herein, simultaneously, in some embodiments this encompasses it with an MIS attached, and in other embodiments without an MIS attached.

Contemplated by this disclosure is any IF1 protein (and/or sequence variant thereof), and use thereof (for at least one use disclosed herein), fragment/sub-sequence thereof (and/or sequence variant thereof), and use thereof (for at least one use disclosed herein), concatenation of fragments thereof (and/or sequence variant thereof), and use thereof (for at least one use disclosed herein).

When a Mitochondrial Import Sequence (MIS) is referred to herein, in preferred embodiments this MIS targets itself, and its attachment(s), to the mitochondrial matrix compartment (as opposed to a different compartment within mitochondria e.g. its Intermembrane Space [IMS]). In further preferred embodiments, the MIS of an IF1 protein is utilized.

All IF1 protein sequences (all sequence versions) in the InterPro “IPR007648” and/or Pfam “PF04568” protein families, and their sub-sequences (and concatenations of their sub-sequences), are componentry to this disclosure, as is their use thereof (for at least one use disclosed herein). To develop some amino acid sequences of this disclosure, I retrieved the proteins, in one-letter amino acid code, from the IPR007648 set and searched (using “regular expression” search in notepad++computer program) for the string “HxxxxxH”, wherein x can be any character (any naturally occurring amino acid) and His histidine (FIG. 10B, residues 36-42 of SEQ ID NO:676), dismissing all the sequences without it. I then manually aligned the remaining sequences to co-correspond/overlap at this motif (aligning the two flanking histidines). Then I made a note of, across all these sequences, which amino acids were found at each position over the entirety of their length. In order to generate a Markush type sequence, a truncated length of which is disclosed herein (SEQ ID NO:1) [in different embodiments the un-truncated length is SEQ ID NO:1 instead (not shown)], within which are some amino acid sequence embodiments of this disclosure, which inhibit/reduce F₁F₀ ATP hydrolysis (for example inside a cell or in an SMP assay of F₁F₀ ATP hydrolysis), and the use of one or more of these (for at least one use disclosed herein) is componentry to this disclosure. Shorter sub-sequences of this Markush type sequence, and the use of one or more of their componentry sequences (for at least one use disclosed herein), are also componentry to this disclosure. Concatenations of shorter sub-sequences of this Markush type sequence, and the use of one or more of their componentry sequences (for at least one use disclosed herein), are also componentry to this disclosure. The method used inherently dismisses some IF1 proteins, from some species, because not all have the “HxxxxxH” motif employed. But they do still have a pH dependence motif. Just with a different residue than histidine at the position that corresponds to the 55^{th} position of “mature” (without MIS) bovine IF1 protein. In such cases their 56^{th} position is typically histidine. So such sequences can be retrieved using the aforementioned method, but wherein the “HxxxxxxH” regular expression is used in the notepad++program instead, wherein x can be any character. In alternative embodiments, componentry to this disclosure, but not shown, such IF1 proteins are not dismissed, as they were, and are also incorporated into SEQ ID NO:1, and SEQ ID NO:2, and any derived sequences of one or both thereof. In other embodiments, the aforementioned method is performed using a different motif(s) characteristic to all/many IF1 proteins instead. Optionally a different aspect(s) to the “pH dependence motif” and/or utilizing other IF1 protein feature(s) shown in FIG. 10.

≙ is symbol for “corresponding to”. SEQ ID NO:2 is the same as SEQ ID NO:1 except that its 175^{th} and 181^{st} residues (corresponding to [≙] H49 and H55 in “mature” human IF1 protein) aren't restricted to being histidine, and the possible scope of its 140^{th}, 152^{nd}, 174^{th}, and 182^{nd} residues (≙S14, E26, H48 and H56 in “mature” human IF1 protein) is broadened, such that each aforementioned residue can be any amino acid coded for by the genetic code. In preferred embodiments, the 175^{th} residue is lysine or alanine or arginine (≙H49K or H49A or H49R), and/or the 152^{nd} residue is glutamine or alanine (≙Q26, E26Q, E26A, or Q26A), and/or one or more of the 140^{th}, 174^{th}, 181^{st} and 182^{nd} residues is alanine (≙one or more of A14/S14A/T14A, H48A/Y48A, H55A, H56A/T56A/S56A).

A sub-sequence/fragment, or concatenated fragments thereof, of SEQ ID NO:2 (or of any sequence herein) is contemplated. A preferred sub-sequence of SEQ ID NO:2 is a sequence comprising (or consisting of) one or more of the following sub-sequences: its residues: 127-210 (≙“mature” bovine IF1 protein), 102-210 (ϑbovine IF1 protein with an N-terminal Mitochondrial Import Sequence [MIS]), 102-207 (≙human/mouse IF1 protein with MIS), 127-207 (≙human/mouse “mature” IF1 protein without MIS), 102-208 (≙rat IF1 protein with MIS), 127-208 (≙rat “mature” IF1 protein without MIS), 140-173 (≙14-47 of bovine IF1 protein, a “minimal inhibitory sequence” [141-142]), 168-184 (≙42-58 of bovine IF1 protein, an alternative “minimal inhibitory sequence” [144-147]), 168-185 (≙42-59 of bovine IF1 protein), 127-182 (1-56 of bovine IF1 protein), 127-183 (≙1-57 of bovine IF1 protein), 127-184 (≙1-58 of bovine IF1 protein), 127-185 (≙1-59 of bovine IF1 protein), 127-186 (≙1-60 of bovine IF1 protein, cannot dimerize, exists as monomer [143]), 137-182 (≙10-56 of bovine IF1 protein), 137-183 (≙10-57 of bovine IF1 protein), 137-184 (≙10-58 of bovine IF1 protein), 137-185 (≙10-59 of bovine IF1 protein), 137-186 (≙10-60 of bovine IF1 protein), 140-172 (14-46 of bovine IF1 protein), 140-171 (14-45 of bovine IF1 protein), 140-170 (≙14-44 of bovine IF1 protein), 140-169 (≙14-43 of bovine IF1 protein), 140-168 (≙14-42 of bovine IF1 protein), 140-167 (≙14-41 of bovine IF1 protein), 139-173 (≙13-47 of bovine IF1 protein), 138-173 (≙12-47 of bovine IF1 protein), 137-173 (≙11-47 of bovine IF1 protein), 136-173 (≙10-47 of bovine IF1 protein), 135-173 (≙9-47 of bovine IF1 protein), 134-173 (≙8-47 of bovine IF1 protein), 133-173 (≙7-47 of bovine IF1 protein), 132-173 (≙6-47 of bovine IF1 protein), 131-173 (≙5-47 of bovine IF1 protein), 130-173 (≙4-47 of bovine IF1 protein), 129-173 (≙3-47 of bovine IF1 protein), 128-173 (≙2-47 of bovine IF1 protein), 127-173 (≙1-47 of bovine IF1 protein), 142-173 (≙16-47 of bovine IF1 protein), 143-173 (≙17-47 of bovine IF1 protein), 127-173 (≙1-47 of bovine IF1 protein), 127-182 (≙1-56 of bovine IF1 protein), 136-172 (≙10-46 of bovine IF1 protein), 141-173 (≙15-47 of bovine IF1 protein), 136-210 (≙10-84 of bovine IF1 protein), 140-210 (≙14-84 of bovine IF1 protein), 140-186 (≙14-60 of bovine IF1 protein), 174-182 (≙ of 48-56 of bovine IF1 protein), 175-181 (≙49-55 of bovine IF1 protein). Componentry to this disclosure is residues 102-126 (≙human/bovine/mouse/rat Mitochondrial Import Sequence [MIS]) concatenated to another sequence of this disclosure (preferably at the N-terminal end of the 2^{nd} sequence) e.g. to residues 140-173 or 168-184. Concatenated sequences of this disclosure are themselves sequences of this disclosure.

SEQ ID NO:3 is residues 102-210 of SEQ ID NO:2 (A-25 {with MIS} to 84 of bovine IF1 protein), optionally wherein 1, 2 or 3 of the C-terminal residues are absent (e.g. to correspond to −25 {with MIS} to 81 of mouse/human IF1 protein), and/or optionally with 1 to 5 aspartic acid (D) residues added on the C-terminal end.

SEQ ID NO:4 is residues 127-210 of SEQ ID NO:2 (≙1 to 84 of bovine IF1 protein), optionally wherein 1, 2 or 3 of the C-terminal residues are absent (e.g. to correspond to 1 to 81 of mouse/human IF1 protein), and/or optionally with 1 to 5 aspartic acid (D) residues added on the C-terminal end.

Some IF1 Proteins, Including Some Novel IF1 Proteins by the Teaching of this Disclosure:

SEQ ID NO:5 is a Markush type sequence, largely within the amino acid sequence space of SEQ ID NO: 1 (but not completely because I found further IF1 proteins, not in the Interpro IPR007648 set, for which this Markush was broadened to encompass, which was especially important because these further IF1 proteins were from absolutely long-lived, and/or long-lived for their size, species), produced from a manual alignment of some IF1 protein sequences, wherein the initiation residue (M) and H49 residue (“mature” protein numbering) of each is in alignment with the initiation (M) and H49 residues respectively of human IF1 protein. The aligned IF1 proteins used to make SEQ ID NO:5 were a subset (those with the aforementioned alignment to human IF1 protein) of those in FIG. 10 (corresponding wild-type sequence IF1 proteins were used instead of mutants).

SEQ ID NO:6 is a consensus type sequence, within the amino acid sequence space of SEQ ID NO:5, which has the most commonly observed residue(s) at each position.

SEQ ID NO:7 is a Markush type sequence encompassing a sub-space of SEQ ID NO:5, encompassing IF1 protein sequences from some long-lived, either absolutely and/or for their size, species (the IF1 protein sequences used are a sub-set of those in FIG. 10P).

A Markush type sequence, and the sequences within it (and use of one or more thereof [for at least one use disclosed herein]), encompassing IF1 protein sequences from more and/or different long-lived species, long-lived either absolutely and/or for their size (wherein the former is more preferred), is componentry to this disclosure.

SEQ ID NO:8 is a consensus type sequence, within the amino acid sequence space of SEQ ID NO:7, which has the most commonly observed residue(s) at each position.

SEQ ID NO:9 is a Markush type sequence encompassing a sub-space of SEQ ID NO:7, encompassing IF1 protein sequences from some long-lived (in absolute terms, not just for their size) species: bowhead, fin, blue, humpback, killer, sperm, gray, Cuvier's beaked, long-finned pilot whales, human, West Indian manatee, African bush elephant, Gorilla.

SEQ ID NO:10 is a consensus type sequence, within the amino acid sequence space of SEQ ID NO:9, which has the most commonly observed residue(s) at each position.

SEQ ID NO:11 is a Markush type sequence encompassing a sub-space of SEQ ID NO:9, encompassing IF1 protein sequences from some very long-lived species: bowhead, fin, blue, whales and human.

SEQ ID NO:12 is a consensus type sequence, within the amino acid sequence space of SEQ ID NO:11, which has the most commonly observed residue(s) at each position. Incidentally, which actually corresponds to “immature” (with Mitochondrial Import Sequence still attached) IF1 protein sequence from bowhead whale.

SEQ ID NO:13 is the same as SEQ ID NO:12 except that its first 25 residues, its Mitochondrial Import Sequence (MIS), is different, being that of SEQ ID NO:5.

SEQ ID NO:14 is the same as SEQ ID NO:12 except that its first 25 residues, its Mitochondrial Import Sequence (MIS), is different, being that of human IF1 protein.

SEQ ID NO:15 is the same as SEQ ID NO:12 except that its first 25 residues, its Mitochondrial Import Sequence (MIS), is different, being that of mouse IF1 protein.

SEQ ID NO:16 is a Markush type sequence that (partially) encompasses all the naturally occurring IF1 protein sequences of FIG. 10 (corresponding wild-type sequence IF1 proteins were used instead of mutants), including:
[a] those sequences whose initiation residue (M) and H49 residue (“mature” IF1 protein numbering) is in simultaneous alignment with those of human IF1 protein and
[b] (in distinction to SEQ ID NO:5) also those sequences whose initiation residue (M) and H49 residue (“mature” protein numbering) do not simultaneously align with those of human IF1 protein, wherein these were aligned against the human IF1 protein, such that their H49 residues aligned, and then, for this sub-set (corresponding to this bullet point [b]), only the IF1 protein part that aligned with the “mature” (without its Mitochondrial Import Sequence [MIS]) human IF1 protein was incorporated into this Markush type sequence.

SEQ ID NO:17 is a Markush type sequence with its first 25 residues being the same as the first 25 residues of SEQ ID NO:5, which corresponds to a Mitochondrial Import Sequence (MIS), attached to a “mature” IF1 protein sequence, which is a Markush type sequence encompassing IF1 protein sequences from some very long-lived species: bowhead, fin, blue, whales, Galápagos tortoise, Eastern box turtle and human.

SEQ ID NO:18 is a consensus type sequence, within the amino acid sequence space of SEQ ID NO:17, which has the most commonly observed residue(s) at each “mature” IF1 protein position.

SEQ ID NO:19 is the same as SEQ ID NO:18 except that its first 25 residues, its Mitochondrial Import Sequence (MIS), is different, being that of human IF1 protein.

SEQ ID NO:20 is the same as SEQ ID NO:18 except that its first 25 residues, its Mitochondrial Import Sequence (MIS), is different, being that of mouse IF1 protein.

SEQ ID NO:21 is the same as SEQ ID NO:16 except that it has the possibility of 1 to 5 more aspartic acid (D) residues at its C-terminal end. Longer living species, as a function of a more potent IF1 protein at normal mitochondrial matrix pH (8), tend to have more aspartic acid residues at the C-terminal end of their IF1 protein.

### Some Sequence Variants Thereof

SEQ ID NO:22 to SEQ ID NO:38 are the same as SEQ ID NO:5 to SEQ ID NO:21 respectively, except that their 74^{th} and 80^{th} residues (corresponding to (A) H49 and H55 in “mature” human IF1 protein) are not restricted to being histidine, and the possible scope of their 39^{th}, 51^{st}, 73^{rd}, and 81^{st} residues (corresponding to (A) S14, E26, H48 and H56 in “mature” human IF1 protein) is broadened, such that each aforementioned residue can be any amino acid coded for by the genetic code. In preferred embodiments, the 74^{th} residue is lysine or alanine or arginine (+H49K or H49A or H49R), and/or the 51^{st} residue is glutamine or alanine (≙Q26, E26Q, E26A, or Q26A), and/or one or more of the 39th, 73^{rd}, 80^{th} and 81^{st} residues is alanine (≙one or more of A14/S14A/T14A, H48A/Y48A, H55A, H56A/T56A/S56A). SEQ ID NO:39 to SEQ ID NO:55 are the same as SEQ ID NO:22 to SEQ ID NO:38 respectively, except that their 74^{th} residue is delimited to histidine, lysine or alanine (+H49/H49K/H49A), their 51^{st} residue is delimited to glutamic acid, glutamine or alanine (+E26/Q26/E26Q/E26A/Q26A), their 39^{th} residue is delimited to serine, threonine or alanine (≙A14/S14/T14/S14A/T14A), their 73^{rd} residue is delimited to histidine, tyrosine or alanine (+H48/Y48/H48A/Y48A), and their 80^{th} and 81^{st} residues are each independently delimited to histidine or alanine (+H55/H55A, H56/H56A/T56A/S56A). In particular preferred embodiments, the 74^{th} residue is lysine (≙H49K) and/or the 39^{th} residue is alanine (≙A14/S14A/T14A). SEQ ID NO:56 to SEQ ID NO:72 are the same as SEQ ID NO:22 to SEQ ID NO: 38 respectively, except that their 74^{th} residue is delimited to lysine (≙H49K). SEQ ID NO:73 to SEQ ID NO:89 are the same as SEQ ID NO:22 to SEQ ID NO:38 respectively, except that their 39th residue is delimited to alanine (≙A14/S14A/T14A). SEQ ID NO:90 to SEQ ID NO:106 are the same as SEQ ID NO:22 to SEQ ID NO:38 respectively, except that their 74^{th} residue is delimited to lysine (H49K) and their 39^{th} residue is delimited to alanine (≙A14/S14A/T14A). SEQ ID NO:107 to SEQ ID NO: 123 are the same as SEQ ID NO:22 to SEQ ID NO:38 respectively, except that their 74^{th} residue is lysine and their 39^{th}, 51^{st}, 73^{rd}, 80^{th} and 81^{st} residues are alanine (≙A14/S14A/T14A, E26A/Q26A, H48A/Y48A, H49K, H55A, H56A/T56A/S56A).

Other embodiments are SEQ ID NO:m, where m is an integer in the range 5-123 (different values of m are different embodiments), shorter at the C-terminal end by a number of residues selected from 1, 2, 3 and 4 residues, and/or with 1 to 5 additional aspartic acid (D) residues at its C-terminal end, and/or with the Mitochondrial Import Sequence (MIS) at the N-terminal end (first 25 residues) absent/replaced with a different MIS/substituted for the MIS of the species being administered to (even if this is longer than 25 residues), preferably the MIS of its native IF1 protein. In preferred embodiments, the MIS used is a MIS from the species being administered to, preferably the MIS of their native IF1 protein.

### CPP Sequences

SEQ ID NO:124 is a Markush type sequence/scheme that encapsulates major Tat sequence variants. With the possibility for a cysteine or glycine/proline (independently selected) at their N- and/or C-terminal end, wherein the former permits a disulphide bond to the cargo sequence (carrying a cysteine, either as part of its sequence naturally or artificially added for this purpose, wherein, if added, its added cysteine is optionally encapsulated in this present Markush scheme—so you will see at its N- and C-terminal ends there is the possibility of two adjacent cysteines, which, unlike the other connections in the Markush scheme, are bound by a disulphide rather than a peptide bond) and the latter can confer flexibility between the CPP and a peptide bonded cargo sequence, wherein in further embodiments, not represented in the Markush, this glycine/proline can actually be a plurality of glycine and/or proline residues up to 10 residues long. In the scheme, next to each possible cysteine residue is the possibility for an aliphatic reside (not in the scheme, but still contemplated, is to have multiple, up to 5 such residues), which can reduce the amount of “disulfide bond exchange” (U.S. Pat. No. 9,255,124B2). Note that in this present scheme, to repeat for stress, when the present residue and an adjacent residue are both cysteine, they are connected by a disulphide, instead of a peptide, bond (if both their side-chains are not being used in a disulfide bond already). One or more cysteine residues in this scheme can be independently substituted for one or more cysteine analogues e.g. penicillamine, alpha-methyl cysteine. One or more L-amino acids in this scheme can be substituted for their corresponding D-amino acid (e.g. D-arginine replaces L-arginine at one or more places). SEQ ID NO:125 is an equivalent Markush type sequence/scheme, with the same accompanying notes, but for Penetratin, SEQ ID NO:126 is that for a poly-Arginine CPP. SEQ ID NO: 124 to SEQ ID NO:126 are the most favoured CPPs for use herein, especially SEQ ID NO:126, and especially its encompassed R7 because of its clinical precedent ([192], U.S. Pat. No. 6,730,293B1), wherein its connection to the cargo sequence via a flanking cysteine and a disulphide bond, or via a peptide bond (optionally via one or more linker/spacer glycine and/or proline residues), is favoured, with peptide bond more favoured. In certain other embodiments, the CPP is selected from the group consisting of the amino acid sequences set forth in SEQ ID NO:440 to SEQ ID NO:638 (and in further embodiments, not shown, one or more of them is embodied with the possibility of one or two flanking cysteines for disulphide attachment to the cargo sequence, optionally with associated aliphatic residue(s), as exemplified with SEQ ID NO:124 to SEQ ID NO:126). In certain other embodiments, the CPP is a different CPP sequence of the art, not presented herein. The CPP component can be any amino acid sequence that facilitates entry of a peptide/protein into a cell. Wherein, in some preferred embodiments, a CPP that accumulates a notable fraction of its cargo to the cytoplasm and/or mitochondrial matrix is preferred.

### Cpp Sequence Concatenated to IF1 Protein (or Sequence Variant Thereof) Sequence

SEQ ID NO:q (or a sub-sequence thereof, or concatenated sub-sequences thereof) concatenated to SEQ ID NO: e (or a sub-sequence thereof, or concatenated sub-sequences thereof), wherein q is an integer selected from the range 124-126 (or from the range 440-638), and e is an integer selected from the range 1-123 (or the more preferred range 5-123), [different values of q and/or e are different embodiments; within their aforementioned range constraints, all possible combinations of q and e integer values are contemplated]. To illustrate with some non-limiting examples:
SEQ ID NO:127 is SEQ ID NO:124 concatenated to SEQ ID NO:38;
SEQ ID NO:128 is SEQ ID NO:125 concatenated to SEQ ID NO:38;
SEQ ID NO:129 is SEQ ID NO:126 concatenated to SEQ ID NO:38;

wherein truncated sequences thereof are contemplated. For non-limiting example, truncated at its C-terminal end by a number (integer) of residues selected from the numerical range 1 to 45 (different integers in different embodiments). For non-limiting example, residues 1-81 of SEQ ID NO:127, 1-84 of SEQ ID NO:128, 1-113 of SEQ ID NO:129, which all correspond to a CPP sequence concatenated to an IF1 protein fragment (≙1-60 bovine IF1 protein).

### Epitope/Affinity Tags:

An epitope/affinity tag sequence is useful for purification of a fusion protein, preferably wherein the tag sequence is at the N-terminal end of the fusion protein and it has a protease cleavage site at, or near, its C-terminal end such that it can be removed by said protease after enabling purification of the fusion protein (e.g. from a cultured unicellular organism(s) recombinantly producing it). SEQ ID NO:130 is HHHHHHDYKDDDDK, which can be cleaved by Enterokinase (cleavage site: DDDDK↓). In alternative embodiments, SEQ ID NO:130 is a different epitope/affinity sequence of the art [N.B. herein epitope and affinity tag are used interchangeably, wherein epitope tag can also refer to affinity tag, and vice-versa, and combination thereof] (e.g. refer to Pina A S, et al. Affinity tags in protein purification and peptide enrichment: An overview. Methods in molecular biology [Clifton, NJ] 1129:147-168). For non-limiting example, selected from poly-histidine, HHHHHH (SEQ ID NO:131), DYKDDDDK (FLAG epitope tag; SEQ ID NO:132), DLYDDDDK (Xpress epitope tag; SEQ ID NO:133), HHHHHHDLYDDDDK (SEQ ID NO:134), DYDDDDK (SEQ ID NO:135), HHHHHHDYDDDDK (SEQ ID NO:136), DTYRYI (SEQ ID NO:137), TDFYLK (SEQ ID NO:138), EQKLISEEDL (SEQ ID NO: 139), EEEEYMPME (SEQ ID NO:140), YPYDVPDYA (SEQ ID NO:141), RYIRS (SEQ ID NO: 142), PPEPET (SEQ ID NO:143), MGGSHHHHHHGMASMTGGQQMGRDLYDDDDKDPSS (SEQ ID NO:144), preferably incorporating (or fused to) a protease cleavage site (and/or a cleavage site cleavable by small molecule treatment) at/near its C-terminal end.

In some embodiments, a cleavable linker sequence is especially favoured between the epitope/affinity tag, situated at the N terminus, and the remainder of the fusion protein, permitting cleavage of the fusion after it has been recovered from its expression/host cell: a fusion protein prepared by a method comprising: (i) culturing a host cell comprising an expression vector comprising a nucleic acid encoding the fusion protein under conditions which provide for expression of said fusion protein encoded by the expression vector within the host cell; and recovering the fusion protein, optionally by way of an epitope/affinity tag sequence component to the fusion protein, optionally wherein this tag is then removed, optionally by the epitope/affinity tag sequence being connected to one end of the fusion protein, optionally the N-terminal end, by a cleavable linker sequence that is cleaved. A cleavable linker region(s) herein can be a protease cleavable linker or other type of cleavable linker of the art e.g. those cleavable by small molecules.

Some non-limiting examples of a cleavage site include Met-X sites, cleavable by cyanogen bromide, Asn-Gly, cleavable by hydroxylamine, Asp-Pro, cleavable by weak acid. But protease cleavage sites are preferred due to the milder cleavage conditions necessary. A non-limiting example is the sequence cut by Enterokinase (DDDDK↓; wherein, incidentally, DDDDK is a part of the FLAG epitope tag: DYKDDDDK [SEQ ID NO:132], and Xpress epitope tag: DLYDDDDK [SEQ ID NO:133]).

Histidine tags have an affinity for nickel/cobalt/zinc/copper/iron ions which have been immobilized by their forming coordinate covalent bonds with a chelator incorporated in the stationary phase. For elution, an excess amount of a compound able to act as a metal ion ligand, such as imidazole, is used. The poly-histidine component of the HHHHHHDYKDDDDK [SEQ ID NO:130] sequence permits isolation of the fusion protein by the aforementioned method, and its DYKDDDDK [residues 7-14 of SEQ ID NO:130] component confers a protease cleavage site for the HHHHHHDYKDDDDK [SEQ ID NO:130] sequence to be cut from the N-terminal end of the fusion protein after it has been isolated e.g. from cultured bacteria/yeast producing it recombinantly. Optionally an epitope/affinity tag is flanked by 1-5 glycines and/or prolines (increases flexibility between domains) at one or both of its ends in a fusion protein of this disclosure.

### Epitope/Affinity Tag Concatenated to CPP Sequence Concatenated to IF1 Protein (or Sequence Variant Thereof) Sequence:

SEQ ID NO:p (or a sub-sequence thereof, or concatenated sub-sequences thereof) concatenated to SEQ ID NO: q (or a sub-sequence thereof, or concatenated sub-sequences thereof) concatenated to SEQ ID NO: e (or a sub-sequence thereof, or concatenated sub-sequences thereof), wherein p is an integer selected from the range 130-144, q is an integer selected from the range 124-126 (or from the range 440-638), and e is an integer selected from the range 1-123 (or the more preferred range 5-123), [different values of p and/or q and/or e are different embodiments; within their aforementioned range constraints, all possible combinations of p, q, and e integer values are contemplated]. To illustrate with a non-limiting example: SEQ ID NO:145 is SEQ ID NO:130 concatenated to SEQ ID NO:126 concatenated to SEQ ID NO: 106;
wherein truncated sequences thereof are also contemplated; indeed, an alternative sequence is SEQ ID NO: 145 without its epitope/affinity tag component i.e. its residues 15-182 as a separate sequence, or a truncation thereof such as its residues 15-113 as a separate sequence; or wherein its CPP component is absent (so in this specific example case, wherein its residues 15-67 are absent and so its 14^{th} residue is directly concatenated to its 68^{th} residue). SEQ ID NO:145 truncated at its C-terminal end by a number (integer) of residues selected from the numerical range 1 to 45 (different integers in different embodiments), optionally additionally truncated at its N-terminal end e.g. such that it is without its epitope/affinity tag component.

In some embodiments, the CPP sequence component is absent and SEQ ID NO:p (or a sub-sequence thereof, or concatenated sub-sequences thereof) is concatenated to SEQ ID NO:e (or a sub-sequence thereof, or concatenated sub-sequences thereof), wherein p is an integer selected from the range 130-145, and e is an integer selected from the range 1-123 (or the more preferred range 5-123), [different values of p and/or e are different embodiments; within their aforementioned range constraints, all possible combinations of p and e integer values are contemplated].

### Some Non-Limiting IF1 Protein Sub-Sequences/Fragments:

First 25 residues (or other residues range) of SEQ ID NO:f concatenated to SEQ ID NO:e (or a sub-sequence thereof, or concatenated sub-sequences thereof), wherein f is an integer selected from the range 1-123 (or the more preferred range 5-123), and wherein e is an integer selected from the range 1-123 (or the more preferred range 5-123); optionally wherein SEQ ID NO:q (or a sub-sequence thereof, or concatenated sub-sequences thereof) is concatenated to the N-terminal end of SEQ ID NO:f in this fusion protein, wherein q is an integer selected from the range 124-126 (or from the range 440-638); optionally wherein SEQ ID NO:p (or a sub-sequence thereof, or concatenated sub-sequences thereof) is concatenated to the N-terminal end of this fusion protein (containing or not containing SEQ ID NO:q), wherein p is an integer selected from the range 130-145, [different values of f and/or e and/or q and/or p are different embodiments; within their aforementioned range constraints, all possible combinations of f, e, q, p integer values are contemplated]; optionally/preferably wherein the SEQ ID NO:e component is a fragment thereof, optionally/preferably being one of its following fragments (different fragments in different embodiments): its residues: u−o, wherein u is equal to or an integer greater than 26, o is equal to or an integer less than its total number of residues, and u<o, wherein to illustrate with some non-limiting examples: residues: 26-109, 27-109, 28-109, 29-109, 30-109, 31-109, 32-109, 33-109, 34-109, 35-109, 36-109, 37-109, 38-109, 39-109, 40-109, 41-109, 42-109, 43-109, 44-109, 45-109, 46-109, 47-109, 48-109, 49-109, 50-109, 51-109, 52-109, 53-109, 54-109, 55-109, 56-109, 57-109, 58-109, 59-109, 60-109, 61-109, 62-109, 63-109, 64-109, 65-109, 66-109, 67-109, 68-109, 69-109, 70-109, 71-109, 72-109, 73-109, 74-109, 75-109, 76-109, 77-109, 78-109, 79-109, 80-109, 81-109, 82-109, 83-109, 84-109, 85-109, 86-109, 87-109, 88-109, 89-109, 90-109, 91-109, 92-109, 93-109, 94-109, 95-109, 96-109, 97-109, 98-109, 99-109, 100-109, 101-109, 102-109, 103-109, 104-109, 105-109, 106-109, 107-109, 108-109, and, for each aforementioned fragment, all possible fragments thereof are also contemplated (e.g. truncated at C-terminus by a number of residues),
wherein to illustrate with some non-limiting examples: residues: 26-85, 35-85, 35-72, 38-72, 39-72, 39-71, 39-70, 39-69, 39-68, 39-67, 67-83, 73-81, 74-80 (≙1-60, 10-60, 10-47, 13-47, 14-47, 14-46, 14-45, 14-44, 14-43, 14-42, 42-58, 48-56, 49-55 of bovine IF1 protein),
wherein to illustrate with some particular non-limiting examples:
residues 1-25 of SEQ ID NO:38 (≙human/bovine/mouse/rat Mitochondrial Import Sequence [MIS]) concatenated to one of the following:
   residues 35-109 of SEQ ID NO:38 (≙10-84 of bovine IF1 protein);
   residues 39-109 of SEQ ID NO:38 (≙14-84 of bovine IF1 protein);
   residues 26-85 of SEQ ID NO:38 (≙1-60 of bovine IF1 protein);
   residues 35-85 of SEQ ID NO:38 (≙10-60 of bovine IF1 protein);
   residues 39-85 of SEQ ID NO:38 (≙14-60 of bovine IF1 protein);
   residues 35-72 of SEQ ID NO:38 (≙10-47 of bovine IF1 protein);
   residues 38-72 of SEQ ID NO:38 (≙13-47 of bovine IF1 protein);
   residues 39-72 of SEQ ID NO:38 (≙14-47 of bovine IF1 protein);
   residues 39-71 of SEQ ID NO:38 (≙14-46 of bovine IF1 protein);
   residues 39-70 of SEQ ID NO:38 (≙14-45 of bovine IF1 protein);
   residues 39-69 of SEQ ID NO:38 (≙14-44 of bovine IF1 protein);
   residues 39-68 of SEQ ID NO:38 (≙14-43 of bovine IF1 protein);
   residues 39-67 of SEQ ID NO:38 (≙14-42 of bovine IF1 protein);
   residues 67-83 of SEQ ID NO:38 (≙42-58 of bovine IF1 protein);
   residues 73-81 of SEQ ID NO:38 (≙48-56 of bovine IF1 protein);
   residues 74-80 of SEQ ID NO:38 (≙49-55 of bovine IF1 protein);
   residues x-y of SEQ ID NO:38 where x is an integer between 26 and 46 (≙between 1 and 20 of bovine IF1 protein), or between 26 and 69 (≙between 1 and 44 of bovine IF1 protein) or between 26 and 109 (≙between 1 and 84 of bovine IF1 protein), and y is an integer between 65 and 110 (≙between 40 and 85 of bovine IF1 protein), or between 75 and 110 (≙between 50 and 85 of bovine IF1 protein) or between 85 and 110 (≙between 60 and 85 of bovine IF1 protein) or between 27 and 110 (≙between 2 and 85 of bovine IF1 protein), [different values of x and/or y are different embodiments; within their aforementioned range constraints, all possible combinations of x and y integer values are contemplated];
   optionally wherein this fusion protein has a Cell Penetrating Peptide (CPP) sequence and/or epitope/affinity tag concatenated to its N-terminal end, wherein to illustrate with some particular non-limiting examples:
SEQ ID NO:146 is SEQ ID NO:130 (example epitope tag) concatenated to SEQ ID NO:124 (example CPP sequence) concatenated to the first 25 residues of SEQ ID NO:38 (Mitochondrial Import Sequence) concatenated to residues 26-85 of SEQ ID NO:38 (≙1-60 of bovine IF1 protein);
   SEQ ID NO:147 is the same but instead of residues 26-85, it has residues 35-85 of SEQ ID NO: 38 (≙10-60 of bovine IF1 protein);
   SEQ ID NO:148 is of the same form but instead has residues 38-72 of SEQ ID NO:38 (≙13-47 of bovine IF1 protein);
   SEQ ID NO:149 is of the same form but instead has residues 39-72 of SEQ ID NO:38 (≙14-47 of bovine IF1 protein);
   SEQ ID NO:150 is of the same form but instead has residues 67-83 of SEQ ID NO:38 (≙42-58 of bovine IF1 protein);
   wherein each of these sequences without its epitope tag and/or its CPP sequence component(s) (and/or wherein a different epitope/affinity tag and/or different CPP sequence is componentry to the sequence) is also contemplated, wherein some non-limiting examples are:
   sub-sequence/fragment of any of SEQ ID NO:146 to SEQ ID NO:150 without its first 14 residues (so without epitope/affinity tag), or without its first 36 residues (so without epitope/affinity tag and CPP sequence), or wherein its residues 15-36 are absent and so its 14^{th} residue is directly concatenated to its 37^{th} residue (so without CPP sequence);
   SEQ ID NO:151 to SEQ ID NO:155 are the same as SEQ ID NO:146 to SEQ ID NO:150 except that their Cell Penetrating Peptide (CPP) sequence component is different, being SEQ ID NO: 126; wherein a sub-sequence/fragment/truncation of any of SEQ ID NO:151 to SEQ ID NO:155 (e.g. without its first 14 residues [so without epitope/affinity tag]) is also contemplated;
   truncated sequences of any of SEQ ID NO:146 to SEQ ID NO:155 are contemplated; for non-limiting example: SEQ ID NO:y, where y is an integer selected from 146, 147, 151, 152, truncated at its C-terminal end by a number (integer) of residues selected from the numerical range 1 to 18, or where y is instead a number (integer) selected from 148, 149, 153, 154, truncated at its C-terminal end by a number (integer) of residues selected from the numerical range 1 to 6; in all these cases optionally with an additional truncation but at its N-terminal end e.g. a N-terminal truncation disclosed above [different values of y, and/or different number of residues truncated, are different embodiments].

SEQ ID NO:156 encompasses the human and mouse Mitochondrial Import Sequence (MIS) for their respective IF1 proteins.

SEQ ID NO:157 is SEQ ID NO:130 (example epitope tag) concatenated to SEQ ID NO:126 (example CPP sequence) concatenated to SEQ ID NO:156 concatenated to residues 26-85 of SEQ ID NO:43 (≙1-60 of bovine IF1 protein) [but a bit broader than SEQ ID NO:43 at its 81^{st} residue wherein Thr and Ser are permitted in addition to His and Ala]; SEQ ID NO:158 is the same but instead of residues 26-85, it has residues 35-85 of SEQ ID NO:43 (≙10-60 of bovine IF1 protein); SEQ ID NO:159 is of the same form but instead has residues 38-72 of SEQ ID NO:43 (≙14-60 of bovine IF1 protein).

SEQ ID NO:160 is SEQ ID NO:130 (example epitope tag) concatenated to SEQ ID NO:126 (example CPP sequence) concatenated to SEQ ID NO:156 concatenated to residues 39-72 of SEQ ID NO:45 (≙14-47 of bovine IF1 protein); SEQ ID NO:161 is the same but instead of residues 38-72, it has residues 67-83 of SEQ ID NO:45 (≙42-58 of bovine IF1 protein).

Truncated sequences thereof are contemplated: for non-limiting example: SEQ ID NO:157 or SEQ ID NO:158 truncated at its C-terminal end by a number (integer) of residues selected from the numerical range 1 to 18 (different integers in different embodiments), SEQ ID NO:45 truncated at its C-terminal end by a number (integer) of residues selected from the numerical range 1 to 6 (different integers in different embodiments), optionally with an additional truncation but at the N-terminal end e.g. as disclosed below.

Sub-sequence/fragment of any of SEQ ID NO:157 to SEQ ID NO:161, e.g. without its first 14 residues (so without epitope/affinity tag), or without its first 67 residues (so without epitope/affinity tag and CPP sequence), or wherein its residues 15-67 are absent and so its 14^{th} residue is directly concatenated to its 68^{th} residue (so without CPP sequence).

SEQ ID NO:162 and SEQ ID NO:163 are human and mouse Mitochondrial Import Sequence (MIS) for their respective IF1 proteins respectively.

SEQ ID NO:164 is an example Tat CPP sequence, with optional flanking glycine/proline residues (optionally contains one or more corresponding D-amino acids). SEQ ID NO:165 is an example poly-arginine CPP sequence, with optional flanking glycine/proline residues (optionally contains one or more corresponding D-amino acids). If it is selected to be just RRRRRRR [SEQ ID NO:455], this has the benefit that this CPP sequence has proven safe in a clinical stage drug candidate (discussed elsewhere herein), and if it is selected to be RRRRRRR [SEQ ID NO:455], RRRRRRRG [SEQ ID NO:461] or RRRRRRRP [residues 4-11 of SEQ ID NO:453], these all have the benefit of being present in naturally occurring human proteins (and some proteins in at least some other mammals), and so have low immunogenicity in humans (and in at least some other mammals).

SEQ ID NO:166 to SEQ ID NO:233 are each SEQ ID NO:130 (example epitope tag) concatenated to SEQ ID NO:164 (example CPP sequence) concatenated to SEQ ID NO:162 (human MIS) concatenated to a “mature” (without MIS) IF1 protein or fragment thereof (wherein some non-limiting key sequence variants are contemplated and encompassed),
which for SEQ ID NO:166 to SEQ ID NO:172 is bovine IF1 protein (or variant thereof) residues: 1-84, 1-60, 10-60, 14-60, 13-47, 14-47, 42-58 respectively,
which for SEQ ID NO:173 to SEQ ID NO:179 is human IF1 protein (or variant thereof) residues: 1-84, 1-60, 10-60, 14-60, 13-47, 14-47, 42-58 respectively,
which for SEQ ID NO:180 to SEQ ID NO:186 is bowhead whale IF1 protein (or variant thereof) residues: 1-84, 1-60, 10-60, 14-60, 13-47, 14-47, 42-58 respectively,
which for SEQ ID NO:187 to SEQ ID NO:192 is blue whale IF1 protein (or variant thereof) residues: 1-84, 1-60, 14-60, 13-47, 14-47, 42-58 respectively (10-60 accidentally omitted but is contemplated also),
which for SEQ ID NO:193 to SEQ ID NO:198 is fin whale IF1 protein (or variant thereof) residues: 1-84, 1-60, 10-60, 14-60, 13-47, 14-47, 42-58 respectively (10-60 accidentally omitted but is contemplated also),
which for SEQ ID NO:199 to SEQ ID NO:205 is Galápagos tortoise (Chelonoidis abingdonii) IF1 protein (or variant thereof) residues: 1-84, 1-60, 10-60, 14-60, 13-47, 14-47, 42-58 respectively,
which for SEQ ID NO:206 to SEQ ID NO:212 is eastern box turtle (Terrapene carolina triunguis) IF1 protein (or variant thereof) residues: 1-84, 1-60, 10-60, 14-60, 13-47, 14-47, 42-58 respectively,
which for SEQ ID NO:213 to SEQ ID NO:219 is naked mole rat IF1 protein (or variant thereof) residues: 1-84, 1-60, 10-60, 14-60, 13-47, 14-47, 42-58 respectively,
which for SEQ ID NO:220 to SEQ ID NO:226 is African bush elephant IF1 protein (or variant thereof) residues: 1-84, 1-60, 10-60, 14-60, 13-47, 14-47, 42-58 respectively, which for SEQ ID NO:227 to SEQ ID NO:233 is West Indian manatee IF1 protein (or variant thereof) residues: 1-84, 1-60, 10-60, 14-60, 13-47, 14-47, 42-58 respectively.

SEQ ID NO:234 to SEQ ID NO:301 are the same as SEQ ID NO:166 to SEQ ID NO:233 except that a different CPP is incorporated: SEQ ID NO:165 instead of SEQ ID NO:164. Any of the aforementioned sequences with a different Mitochondrial Import Sequence (MIS) is contemplated. For illustrative non-limiting example, SEQ ID NO:302 to SEQ ID NO:335 have the MIS of mouse, instead of human: SEQ ID NO: 163 instead of SEQ ID NO:162.

Any sub-sequence/fragment of any of SEQ ID NO:166 to SEQ ID NO:233, e.g. without its first 14 residues (so without epitope/affinity tag), or without its first 27 residues (so without epitope/affinity tag and CPP sequence), or wherein its residues 15-27 are absent and so its 14^{th} residue is directly concatenated to its 28^{th} residue (so without CPP sequence).

Any sub-sequence/fragment of any of SEQ ID NO:234 to SEQ ID NO:335, e.g. without its first (most N-terminal) 14 residues (so without epitope/affinity tag), or without its first 23 residues (so without epitope/affinity tag and CPP sequence), or wherein its residues 15-23 are absent and so its 14^{th} residue is directly concatenated to its 24^{th} residue (so without CPP sequence).

SEQ ID NO:y, where y is an integer selected from 166, 173, 180, 187, 193, 199, 206, 213, 220, 227, 234, 241, 248, 255, 261, 267, 274, 281, 288, 295, 302, 309, 316, 323, 329, truncated at its C-terminal end by a number (integer) of residues selected from the numerical range 1 to 45, or where y is instead an integer selected from 168, 169, 175, 176, 182, 183, 189, 195, 201, 202, 208, 209, 215, 216, 222, 223, 229, 230, 236, 237, 243, 244, 250, 251, 257, 263, 269, 270, 276, 277, 283, 284, 290, 291, 297, 298, 304, 305, 311, 312, 318, 319, 325, 331, 332, truncated at its C-terminal end by a number (integer) of residues selected from the numerical range 1 to 18, or where y is instead an integer selected from 170, 171, 177, 178, 184, 185, 190, 191, 196, 197, 203, 204, 210, 211, 217, 218, 224, 225, 231, 232, 238, 239, 245, 246, 252, 253, 258, 259, 264, 265, 270, 271, 278, 279, 285, 286, 292, 293, 299, 300, 306, 307, 313, 314, 320, 321, 326, 327, 333, 334, truncated at its C-terminal end by a number (integer) of residues selected from the numerical range 1 to 6; in all these cases optionally with an additional truncation but at its N-terminal end e.g. an aforementioned N-terminal truncation [different values of y, and/or different number of residues truncated, are different embodiments].

Aforementioned sequences with a different epitope/affinity tag and/or different Cell Penetrating Peptide (CPP) sequence and/or different Mitochondrial Import Sequence (MIS, wherein in some preferred embodiments the MIS is that which a species to be administered uses for its native IF1 protein) and/or different IF1 protein/fragment (or sequence variant thereof), wherein any IF1 protein is contemplated, or any fragment thereof, wherein IF1 protein/fragment (or sequence variant thereof) from a long-lived species (high maximal lifespan) is especially favoured, wherein any component(s) may be absent except the IF1 protein/fragment (or sequence variant thereof), optionally wherein all is absent except this.

### A General Non-Limiting Formula: N to C-Terminal:

### [Epitope/Affinity Tag (or Absent)]-[Cell Penetrating Peptide Sequence (or Absent)]-[Mitochondrial Import Sequence (or Absent)]-[IF1 Protein/Fragment or Sequence Variant Thereof (NEVER Absent)].

In sequences herein in which an epitope/affinity tag is concatenated to a Cell Penetrating Peptide (CPP) sequence, in alternative fusion protein embodiments (not shown), the CPP sequence is absent and the epitope/affinity tag is concatenated directly to the Mitochondrial Import Sequence (MIS) of the IF1 protein/fragment (or sequence variant thereof) containing fusion protein.

Disclosed herein are some illustrative non-limiting examples of epitope/affinity tags, CPP sequences, MIS sequences, IF1 proteins/fragments (and sequence variants thereof). From the teaching of this disclosure, one of the art knows how to assemble further sequences of this disclosure, which are not explicitly shown. To illustrate, SEQ ID NO:157 has a CPP component which is SEQ ID NO:126, but one of the art will see that this can be substituted with a different CPP sequence of the art to produce an alternative sequence of this disclosure, optionally with a different CPP sequence mentioned herein, e.g. SEQ ID NO:125.

Any sequence (or fragment/concatenated fragments thereof) of SEQ ID NO:166 to SEQ ID NO: 335 wherein its CPP component is substituted with a different CPP sequence (or multiple thereof), optionally selected from SEQ ID NO:124 to SEQ ID NO: 126 and/or from SEQ ID NO:440 to SEQ ID NO: 638.

Componentry to this disclosure is an IF1 protein, or fragment thereof, from one species attached to the C-terminal end of a Mitochondrial Import Sequence (MIS) from a different species, preferably the MIS that the species uses for its native IF1 protein.

For a concatenated sequence herein, every possible arrangement of its concatenated parts relative to one another is contemplated and componentry to this disclosure e.g. one or more concatenated parts incorporated in the opposite orientation than shown, and/or a different ordering of componentry concatenations, and/or wherein one or more of the concatenated sequences is absent. Componentry to this disclosure is SEQ ID NO:X (or fragment thereof, or concatenated fragments thereof) concatenated to SEQ ID NO:X (or fragment thereof, or concatenated fragments thereof) optionally, in turn, further concatenated to SEQ ID NO:X (or fragment thereof, or concatenated fragments thereof) optionally, in turn, further concatenated to SEQ ID NO:X (or fragment thereof, or concatenated fragments thereof) optionally, in turn, further concatenated to SEQ ID NO:X (or fragment thereof, or concatenated fragments thereof), wherein X can, independently selected in each case of using X, be 1, or the number of sequences in the Sequence Listing component to this application, or any integer between 1 and the total number of sequences in the Sequence Listing component to this application (wherein each componentry sequence can be in either orientation). Concatenated sequences of this disclosure are themselves sequences of this disclosure, optionally wherein up to ten (or five, or four) sequences are concatenated.

In some embodiments, an amino acid/peptide/protein sequence herein is substituted for a polynucleotide sequence coding for it, which can in turn be concatenated/bound to other (amino acid/polynucleotide) sequence(s) herein e.g. it is within the scope of this disclosure to have a polynucleotide sequence, encoding at least one IF1 protein/fragment (or sequence variant thereof), combined/conjoined (e.g. covalently bound) with at least one Cell Penetrating Peptide (CPP) amino acid sequence (or multiple thereof).

Incidentally, SEQ ID NO:336 is the IF1 protein, with its N-terminal MIS, of bowhead whale (Balaena mysticetus). SEQ ID NO:336 is the IF1 protein, with its N-terminal Mitochondrial Import Sequence [MIS], of blue whale (Balaenoptera musculus). These sequences have never been reported before. These protein sequences and nucleotide sequences that code for them are componentry to this disclosure (optionally isolated/produced/purified/substantially purified/partially purified).

### More Non-Limiting IF1 Protein Sub-Sequences/Fragments:

Some preferred embodiments of this disclosure are (and further embodiments are the use thereof, for at least one use disclosed herein), wherein the IF1 protein referred to below can be at least one IF1 protein sequence variant, e.g. one or more disclosed herein, or any naturally occurring IF1 protein(s), from any species, wherein in some embodiments the IF1 protein is from a mammal such as (without limitation) a cow, rat, mouse, rodent, naked mole rat, primate, human, wherein in some preferred embodiments it is from a large/long-lifespan/long-lifespan for its size/thermoconformer/cold-blooded species, and/or a species that has negligible senescence, and/or from a species with a longer maximal/typical lifespan than the species that the IF1 protein/fragment (or sequence variant thereof) is being administered to:
Protein comprising (or consisting of) one or more of residues
14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 10-84, 14-84, 14-60, 18-84, 10-50, 1-45, 42-56, 42-47, 48-56, 49-55 of a “mature” IF1 protein;
Fusion protein comprising (or consisting of) a Mitochondrial Import Sequence (MIS) concatenated to one or more of residues
14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 10-84, 14-84, 14-60, 18-84, 10-50, 1-45, 42-56, 42-47, 48-56, 49-55 of a “mature” IF1 protein;
Fusion protein comprising (or consisting of) CPP sequence concatenated to Mitochondrial Import Sequence (MIS) concatenated to one or more of residues
14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 10-84, 14-84, 14-60, 18-84, 10-50, 1-45, 42-56, 42-47, 48-56, 49-55 of a “mature” IF1 protein;
Fusion protein comprising (or consisting of) epitope/affinity tag sequence concatenated to CPP sequence concatenated to Mitochondrial Import Sequence (MIS) concatenated to one or more of residues
14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 10-84, 14-84, 14-60, 18-84, 10-50, 1-45, 42-56, 42-47, 48-56, 49-55 of a “mature” IF1 protein.

This is illustrated herein using IF1 protein from some species, but in different embodiments, a different IF1 protein from a different species (or sequence variant thereof) is selected instead, and the equivalent sub-sequences, concatenated sub-sequences and fusion proteins (and their use thereof, for at least one use disclosed herein) are componentry to this disclosure.

Other IF1 Proteins/Fragments, from Other Species, and Fusion Proteins Thereof

With the teaching of this disclosure in hand, for example seeing the form of sequences in its Sequence Listing component, other peptide/protein sequences of this disclosure (and coding polynucleotides thereof, and vectors, cells, gene therapies, transgenic organisms thereof; and use of one or more thereof [for at least one use disclosed herein]), not explicitly stated in this disclosure, will be apparent to one of the art e.g, wherein an IF1 protein/fragment (or sequence variant thereof) from a different, unmentioned species is utilized instead, optionally a fusion protein thereof. Some further IF1 protein sequences are presented in FIG. 10 herein. More IF1 protein sequences can be sourced from InterPro family “Mitochondrial ATPase inhibitor (IPR007648)” and/or Pfam family “IATP (PF04568)”. Wherein given the teaching of this disclosure, one of the art can see how to utilize these extra IF1 protein sequences to arrive at further sequences of this disclosure. Innovatively, IF1 protein sequence(s) from one or more long-lived species (either absolutely and/or for its size) is favoured. Some notable embodiments (with maximal lifespan in brackets, if value is available in AnAge online database [AnAge Database of Animal Ageing and Longevity [115]]) utilize an IF1 protein/fragment (or sequence variant thereof) from one or more of a whale species, long-lived mammal species, species with a high maximal lifespan (e.g. as reported by AnAge online database), North Atlantic right whale (67), North Pacific right whale, Southern right whale (70), Greenland shark (392), Rougheye rockfish (205), Tiger rockfish (116), Orange roughy (149), Atlantic sturgeon (60), Tuatara (90), Olm/European cave salamander (102), Eurasian eagle-owl (68), Pink cockatoo (83), Asiatic elephant (66), Hippopotamus (61), Black rhinoceros (49), African Cape buffalo (33), Desert tortoise (63).

A method of this disclosure is to assemble a Markush type scheme encompassing different IF1 proteins from different species, aligned by features of their “pH dependence motif”, wherein this method has been illustrated herein; optionally/preferably/exclusively encompassing some IF1 proteins from some long-lived species, with a high maximal lifespan for their size and/or [more preferred] in absolute terms; and extracting one or more novel IF1 protein sequence variants, and/or one or more fragments thereof, from within its encompassed sequence space, wherein each novel sequence outputted by this method is componentry to this disclosure, as is its use thereof, for at least one use disclosed herein.

### Rehash

Any peptide/protein/amino acid sequence of/in this disclosure concatenated at either end, optionally via a linker sequence (e.g. by one or more glycine and/or proline residues, which confers flexibility), to a Mitochondrial Import Sequence (MIS) is componentry to this disclosure, as is its use thereof (for at least one use disclosed herein), wherein preferably such a sequence only has a single componentry MIS sequence.

Any peptide/protein/amino acid sequence of/in this disclosure concatenated at either end, optionally via a linker sequence (e.g. by one or more glycine and/or proline residues, which confers flexibility), to a Cell Penetrating Peptide (CPP) sequence of the art, and/or a Mitochondria Penetrating Peptide (MPP) sequence of the art, in either orientation (N to C, C to N), is componentry to this disclosure, as is its use thereof (for at least one use disclosed herein), wherein preferably (but not restrictively) such a sequence only has a single componentry CPP or MPP sequence.

Any amino acid sequence of/in this disclosure concatenated at either end, optionally via a linker sequence (e.g. by one or more glycine and/or proline residues, which confers flexibility, e.g. by a cleavable linker sequence, e.g. by a protease cleavable linker or other type of cleavable linker of the art e.g. those cleavable by a small molecule [s]), to an epitope/affinity tag sequence (many known to those of the art), in either orientation, is componentry to this disclosure, as is its use thereof (for at least one use disclosed herein), wherein preferably such a sequence only has a single epitope/affinity tag.

### A Quick Important Point on Scope

14-47 is NOT the only favoured IF1 protein fragment. Herein, 14-47 is cited/used often as an exemplary IF1 protein fragment. Herein, at every point that a 14-47 IF1 protein fragment is referred to/shown, in alternative embodiments, a different IF1 protein fragment (optionally from same species/group of species as the context, or a different species/group of species as the context, or any species) is substituted in its place, for non-limiting example, selected from the group comprising: 42-58, 1-56, 1-60, 10-56, 10-60, 1-58, 10-58, 10-84 (or 42-56 or 42-47). IF1 protein fragment 10-60 is particularly prized, and for every fusion protein herein that comprises a 14-47 IF1 protein fragment, in alternative embodiments this 14-47 component is substituted with the 10-60 equivalent.

Sequences herein that have ≙K49 (using “mature” [without MIS] IF1 protein numbering), incidentally which is the result of H49K substitution from an IF1 protein/fragment sequence, in alternative sequences embodiments (not shown) this residue is alanine or arginine (≙49 or R49) instead; for example this can be applied to some sequences within FIG. 10.

### Some Retroinverse (Retro-Inverse) Embodiments

For a sequence of L-amino acids, its retroinverse/retro-inverse sequence is this sequence in reverse, wherein all the amino acids are D-amino acids. So, it is a sequence reversed with chirality inverted. A retroinverse sequence is more resistant to proteases, and so has a longer half-life in blood. A partially/completely retroinverse sequence of an amino acid/peptide/protein sequence of/in this disclosure is, in turn, a sequence of this disclosure. The retroinverse sequence of SEQ ID NO:X (or a fragment thereof, or concatenated fragments thereof), or partially retroinverse sequence of SEQ ID NO:X (or a fragment thereof, or concatenated fragments thereof) wherein only part(s) of it is retroinverse, wherein X can be 1, or the number of sequences in the Sequence Listing component to this application, or any integer between 1 and the total number of sequences in the Sequence Listing component to this application.

In some embodiments, all the sequence is retroinverse, or a different pattern of retroinverse part(s) applies.

A peptide/protein comprising (or consisting of) at least one partially/completely retroinverse sequence of an IF1 protein/fragment (or sequence variant thereof) (wherein any IF1 protein, from any species, is contemplated).

Contemplated is the retroinverse sequence of a fusion protein sequence comprising (or consisting of): Cell Penetrating Peptide sequence (or absent)]−[Mitochondrial Import Sequence (or absent)]−[IF1 protein/fragment or sequence variant thereof (NEVER absent)]. Or wherein the CPP component (if present) is retroinverse (partially or, more preferably, completely), the MIS component (if present) is not, and the IF1 protein/fragment (or sequence variant thereof) is retroinverse (partially or, more preferably, completely).

The retroinverse sequence of the following sequence: first 25 residues (or other residues range) of SEQ ID NO: f concatenated to SEQ ID NO:e (or a sub-sequence thereof, or concatenated sub-sequences thereof), wherein f is an integer selected from the range 1-123 (or the more preferred range 5-123), and wherein e is an integer selected from the range 1-123 (or the more preferred range 5-123); optionally wherein SEQ ID NO:q (or a sub-sequence thereof, or concatenated sub-sequences thereof) is concatenated to the N-terminal end of SEQ ID NO:f in this fusion protein, wherein q is an integer selected from the range 124-126 (or from the range 440-638), [different values of f and/or e and/or q are different embodiments; within their aforementioned range constraints, all possible combinations of f, e, q integer values are contemplated]; optionally/preferably wherein the SEQ ID NO:e component is a fragment thereof, optionally/preferably being one of its following fragments (different fragments in different embodiments): its residues: u−o, wherein u is equal to or an integer greater than 26, o is equal to or an integer less than its total number of residues, and u<o, wherein to illustrate with some non-limiting examples: residues: 26-109, 27-109, 28-109, 29-109, 30-109, 31-109, 32-109, 33-109, 34-109, 35-109, 36-109, 37-109, 38-109, 39-109, 40-109, 41-109, 42-109, 43-109, 44-109, 45-109, 46-109, 47-109, 48-109, 49-109, 50-109, 51-109, 52-109, 53-109, 54-109, 55-109, 56-109, 57-109, 58-109, 59-109, 60-109, 61-109, 62-109, 63-109, 64-109, 65-109, 66-109, 67-109, 68-109, 69-109, 70-109, 71-109, 72-109, 73-109, 74-109, 75-109, 76-109, 77-109, 78-109, 79-109, 80-109, 81-109, 82-109, 83-109, 84-109, 85-109, 86-109, 87-109, 88-109, 89-109, 90-109, 91-109, 92-109, 93-109, 94-109, 95-109, 96-109, 97-109, 98-109, 99-109, 100-109, 101-109, 102-109, 103-109, 104-109, 105-109, 106-109, 107-109, 108-109, and, for each aforementioned fragment, all possible fragments thereof (e.g. truncated at C-terminus by a number of residues) are also contemplated,
wherein to illustrate with some non-limiting examples: residues: 26-85, 35-85, 35-72, 38-72, 39-72, 39-71, 39-70, 39-69, 39-68, 39-67, 67-83, 73-81, 74-80 (≙1-60, 10-60, 10-47, 13-47, 14-47, 14-46, 14-45, 14-44, 14-43, 14-42, 42-58, 48-56, 49-55 of bovine IF1 protein),
   wherein to illustrate with some non-limiting examples: SEQ ID NO:338 to SEQ ID NO:353 are each the retroinverse sequence of the sequence: SEQ ID NO:164 (example CPP sequence) concatenated to SEQ ID NO:162 (human MIS) concatenated to a “mature” (without MIS) IF1 protein or fragment thereof (wherein some non-limiting key sequence variants are contemplated and encompassed),
   which for SEQ ID NO:338 to SEQ ID NO:341 is bovine IF1 protein (or variant thereof) residues: 10-60, 14-60, 14-47, 42-58 respectively,
   which for SEQ ID NO:342 to SEQ ID NO:345 is human IF1 protein (or variant thereof) residues: 10-60, 14-60, 14-47, 42-58 respectively,
   which for SEQ ID NO:346 to SEQ ID NO:349 is bowhead whale IF1 protein (or variant thereof) residues: 10-60, 14-60, 14-47, 42-58 respectively,
   which for SEQ ID NO:350 to SEQ ID NO:353 is blue whale IF1 protein (or variant thereof) residues: 10-60, 14-60, 14-47, 42-58 respectively.
SEQ ID NO:354 to SEQ ID NO:369 are the same as SEQ ID NO:338 to SEQ ID NO:353 except that a different CPP is incorporated (SEQ ID NO:165 instead of SEQ ID NO:164). SEQ ID NO:370 to SEQ ID NO: 373 are of the same form, using SEQ ID NO:165 as the CPP component, and an IF1 protein fragment from naked mole rat. Any of the aforementioned sequences with a different Mitochondrial Import Sequence (MIS) is contemplated. For illustrative non-limiting example, SEQ ID NO:374 to SEQ ID NO:393 are the same as SEQ ID NO:354 to SEQ ID NO:373 except they have the MIS of mouse, instead of human: SEQ ID NO:163 instead of SEQ ID NO:162.

Truncated forms/fragments of these sequences are contemplated. For non-limiting example, any of SEQ ID NO:338 to SEQ ID NO:353 without its last (most C-terminal) 13 residues (i.e. without its CPP component, without its SEQ ID NO:164 component), or any of SEQ ID NO:354 to SEQ ID NO:373 without its last (most C-terminal) 9 residues (i.e. without its CPP component, without its SEQ ID NO:165 component). SEQ ID NO:y, where y is an integer selected from 338, 339, 342, 343, 346, 347, 351, 352, 354, 355, 358, 359, 362, 363, 366, 367, 370, 371, 374, 375, 378, 379, 382, 383, 386, 387, 390, 391, truncated at its N-terminal end by a number (integer) of residues selected from the numerical range 1 to 18, or where y is instead an integer selected from 340, 344, 348, 352, 356, 360, 364, 368, 372, 376, 380, 384, 388, 392, truncated at its N-terminal end by a number (integer) of residues selected from the numerical range 1 to 6, optionally with an additional truncation but at its C-terminal end e.g. an aforementioned C-terminal truncation [different values of y, and/or different number of residues truncated, are different embodiments].

As an alternative to the above, the following sequence: N to C-terminal order: first 25 residues (or other residues range) of SEQ ID NO:f (normal or partially/completely retroinverse sequence thereof) concatenated to normal or partially/completely retroinverse sequence of SEQ ID NO:e (or a sub-sequence thereof, or concatenated sub-sequences thereof), wherein f is an integer selected from the range 1-123 (or the more preferred range 5-123), and wherein e is an integer selected from the range 1-123 (or the more preferred range 5-123); optionally wherein normal or partially/completely retroinverse sequence of SEQ ID NO:q (or a sub-sequence thereof, or concatenated sub-sequences thereof) is concatenated to the N-terminal end of SEQ ID NO:f in this fusion protein, wherein q is an integer selected from the range 124-126 (or from the range 440-638), [different values of f and/or e and/or q are different embodiments; within their aforementioned range constraints, all possible combinations of f, e, q integer values are contemplated]; optionally/preferably wherein the SEQ ID NO:e component is a fragment thereof, optionally/preferably being one of its following fragments (different fragments in different embodiments): its residues: u−o, wherein u is equal to or an integer greater than 26, o is equal to or an integer less than its total number of residues, and u<o, wherein to illustrate with some non-limiting examples: residues: 26-109, 27-109, 28-109, 29-109, 30-109, 31-109, 32-109, 33-109, 34-109, 35-109, 36-109, 37-109, 38-109, 39-109, 40-109, 41-109, 42-109, 43-109, 44-109, 45-109, 46-109, 47-109, 48-109, 49-109, 50-109, 51-109, 52-109, 53-109, 54-109, 55-109, 56-109, 57-109, 58-109, 59-109, 60-109, 61-109, 62-109, 63-109, 64-109, 65-109, 66-109, 67-109, 68-109, 69-109, 70-109, 71-109, 72-109, 73-109, 74-109, 75-109, 76-109, 77-109, 78-109, 79-109, 80-109, 81-109, 82-109, 83-109, 84-109, 85-109, 86-109, 87-109, 88-109, 89-109, 90-109, 91-109, 92-109, 93-109, 94-109, 95-109, 96-109, 97-109, 98-109, 99-109, 100-109, 101-109, 102-109, 103-109, 104-109, 105-109, 106-109, 107-109, 108-109, and, for each aforementioned fragment, all possible fragments thereof are also contemplated (e.g. truncated at the C-terminus by a number of residues),
wherein to illustrate with some non-limiting examples: residues: 26-85, 35-85, 35-72, 38-72, 39-72, 39-71, 39-70, 39-69, 39-68, 39-67, 67-83, 73-81, 74-80 (+1-60, 10-60, 10-47, 13-47, 14-47, 14-46, 14-45, 14-44, 14-43, 14-42, 42-58, 48-56, 49-55 of bovine IF1 protein),
wherein to illustrate with some non-limiting examples: SEQ ID NO:394 to SEQ ID NO:406 each have the retroinverse sequence of SEQ ID NO:164 (example CPP sequence) concatenated to SEQ ID NO:162 (human MIS, NOT retroinverse) concatenated to the retroinverse sequence of a “mature” (without MIS) IF1 protein or fragment thereof (wherein some non-limiting key sequence variants are contemplated and encompassed),
which for SEQ ID NO:394 to SEQ ID NO:396 is bovine IF1 protein (or variant thereof) residues: 10-60, 14-47, 42-58 respectively (14-60 also contemplated but not shown),
which for SEQ ID NO:397 to SEQ ID NO:399 is human IF1 protein (or variant thereof) residues: 10-60, 14-47, 42-58 respectively (14-60 also contemplated but not shown),
which for SEQ ID NO:400 to SEQ ID NO:402 is bowhead whale IF1 protein (or variant thereof) residues: 10-60, 14-47, 42-58 respectively (14-60 also contemplated but not shown),
which for SEQ ID NO:403 to SEQ ID NO:406 is blue whale IF1 protein (or variant thereof) residues: 10-60, 14-60, 14-47, 42-58 respectively.

SEQ ID NO:407 to SEQ ID NO:419 are the same as SEQ ID NO:394 to SEQ ID NO:406 except that a different CPP is incorporated (SEQ ID NO:165 instead of SEQ ID NO:164). SEQ ID NO:420 to SEQ ID NO:422 are of the same form, using SEQ ID NO:165 as the CPP component, and an IF1 protein fragment from naked mole rat. Any of the aforementioned sequences with a different Mitochondrial Import Sequence (MIS) is contemplated. For illustrative non-limiting example, SEQ ID NO:423 to SEQ ID NO:438 are the same as SEQ ID NO:407 to SEQ ID NO:422 except they have the MIS of mouse, instead of human: SEQ ID NO:163 instead of SEQ ID NO:162.

Truncated forms/fragments of these sequences are contemplated. For non-limiting example, any of SEQ ID NO:394 to SEQ ID NO:406 without its first 13 residues (i.e. without its CPP component, without its SEQ ID NO:164 component), or any of SEQ ID NO:407 to SEQ ID NO:422 without its first 9 residues (i.e. without its CPP component, without its SEQ ID NO:165 component). SEQ ID NO:y, where y is an integer selected from 394, 397, 400, 403, 404, 407, 410, 413, 416, 417, 420, 423, 426, 429, 432, 433, 436, with a number (integer) of residues, selected from the numerical range 1 to 18, deleted within the residue range 39 to 56, or where y is instead an integer selected from 395, 398, 401, 405, 408, 411, 414, 418, 421, 424, 427, 430, 434, 437, with a number (integer) of residues, selected from the numerical range 1 to 6, deleted within the residue range 39 to 44, optionally with an additional truncation but at its N-terminal end e.g. an aforementioned N-terminal truncation [different values of y, and/or different number of residues truncated, are different embodiments].

Any sequence (or fragment/concatenated fragments thereof) of SEQ ID NO:338 to SEQ ID NO:438 wherein its CPP component is substituted with a different CPP sequence (or multiple thereof), optionally selected from SEQ ID NO:124 to SEQ ID NO:126, and/or from SEQ ID NO:440 to SEQ ID NO:638, or the retroinverse (partially/completely) thereof.

### Some Esterified Embodiments

A peptide/protein comprising (or consisting of) at least one IF1 protein/fragment (or sequence variant thereof) with one or more of its carboxyl groups esterified (wherein any IF1 protein, from any species, is contemplated).

In some embodiments, SEQ ID NO:X (or fragment thereof, or concatenated fragments thereof) has one or more of its carboxyl groups esterified, wherein X can be 1, or the number of sequences in the Sequence Listing component to this application, or any integer between 1 and the total number of sequences in the Sequence Listing component to this application.

To illustrate with a non-limiting example (presented here and not in the Sequence Listing because esterification of a carboxyl group(s) cannot be represented within the WIPO ST.25 standard): Human Mitochondrial Import Sequence (MIS, for IF1 protein) concatenated to (using “mature” {without MIS} human IF1 protein numbering) residues 14-47 of human IF1 protein, which has an S14A substitution, with the option of zero or more of its carboxyl groups esterified:

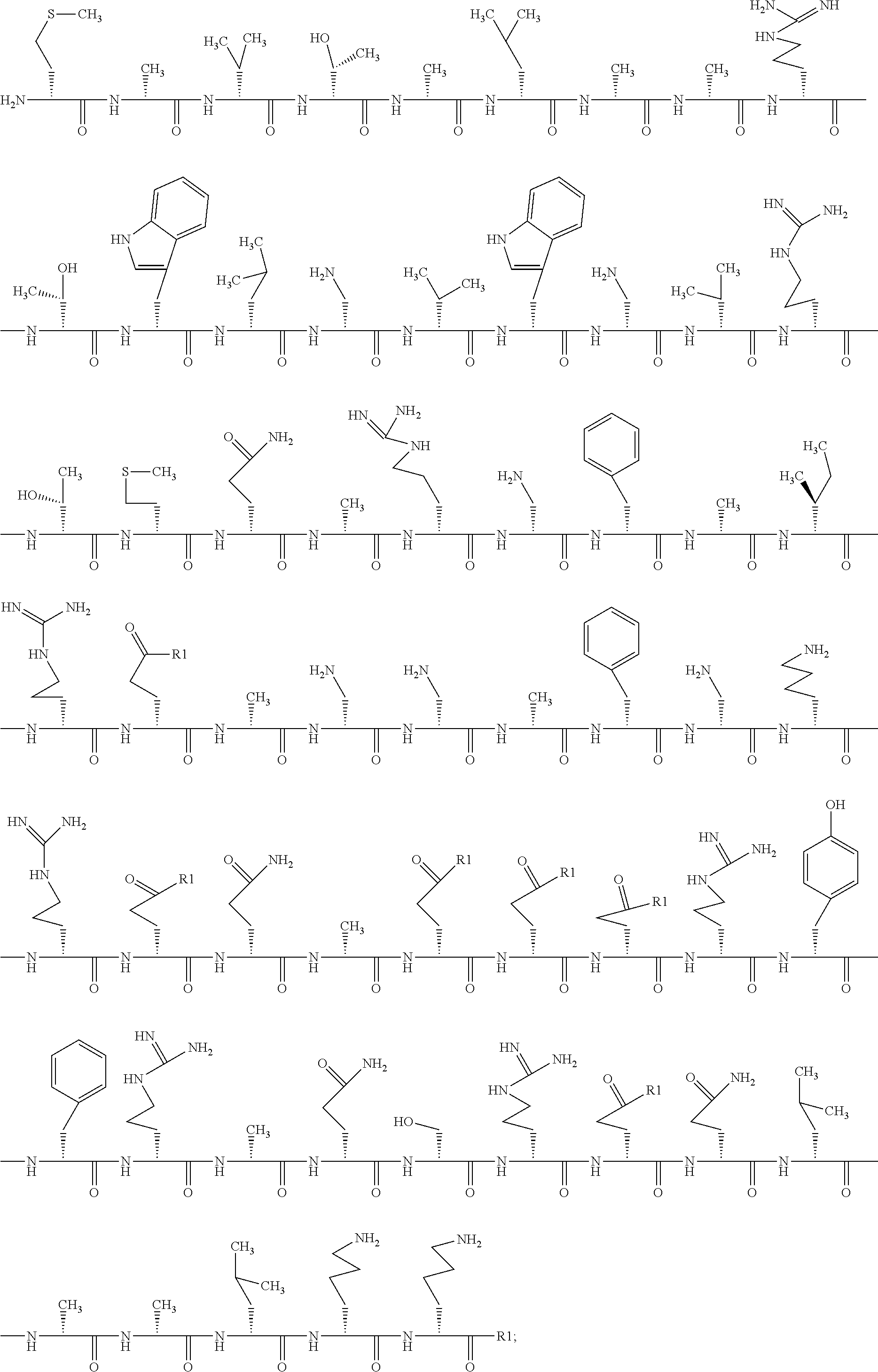

wherein R¹ is selected (independently selected at each incidence of R1) from oxygen (O) or

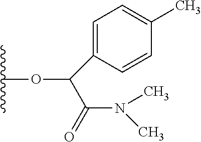 [SMILES: *OC(C(=O)N(C)C)c1ccc(C)cc1]

or a different group used to esterify (esterification) a carboxyl group(s) in one or more of U.S. Pat. Nos. 9,790,483B2, 10,258,695B2, 10,428,323B2, 10,577,303B1, US2020/0032238A1, [193-194].

Below is as immediately above, but, wherein the IF1 protein 14-47 residues component is retroinverse/retro-inverse (reverse sequence, opposite chirality):

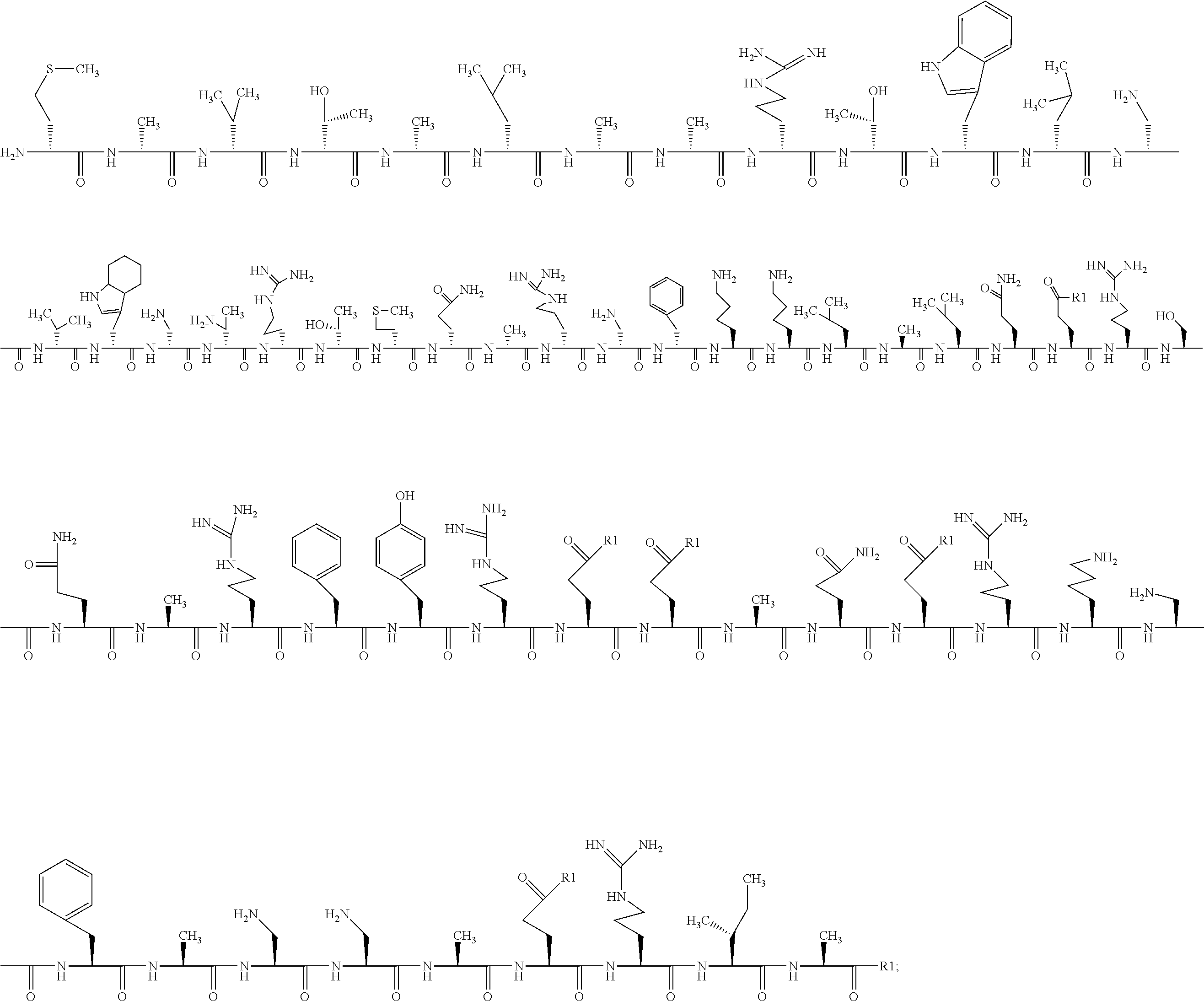

wherein R1 is (independently at each incidence) as defined earlier in this section (under the present heading). In alternative embodiments (not shown) the sequence is all retroinverse (i.e. MIS component as well), or a different sequence section(s) is selected to be retroinverse, and/or a MIS from a different species is used instead (e.g. from mouse) and/or the 14-47 IF1 protein component is from a different species instead (e.g. bowhead or blue or fin whale), wherein optionally, if the 14^{th} residue isn't already alanine, it is substituted to be so, optionally wherein 42-58 (or 1-60 or 10-60) IF1 protein residues are used instead of 14-47, wherein with all these aforementioned options, esterification of zero or more of their carboxyl groups is contemplated (wherein, in preferred embodiments, this can facilitate passage across biological membrane(s), conferring cellular penetration, wherein the ester bond joined moiety, or plurality thereof, is cleaved off by intracellular esterases once inside the cell). Esterification of a peptide/protein can negate its need for fusion of a Cell Penetrating Peptide (CPP) to enter a cell. Herein, where a peptide/protein sequence is presented with a CPP component(s), in alternative embodiments, this CPP component(s) is absent and the peptide/protein is esterified at one or more of its carboxyl groups. In other embodiments, the CPP component(s) remains present and the peptide/protein is esterified at one or more of its carboxyl groups, further ensuring that this peptide/protein can enter the cell. An amino acid/peptide/protein sequence of/in this disclosure which is esterified at one more of its carboxyl groups is componentry to this disclosure.
Modification of N- and/or C-Terminal Ends, and/or Esterified

A peptide/protein comprising (or consisting of) at least one IF1 protein/fragment (or sequence variant thereof) which is modified at its N- and/or C-terminal ends, optionally wherein a lipidic/lipid (e.g. fatty acid) moiety is conjugated/acylated to the N-terminus, and/or with one or more of its carboxyl groups esterified (wherein any IF1 protein, from any species, is contemplated).

A protein comprising (or consisting of) at least one IF1 protein/fragment (or sequence variant thereof) with at least one conjugated/covalently bound lipidic/lipid (e.g. fatty acid) moiety, and/or with one or more of its carboxyl groups esterified (wherein any IF1 protein, from any species, is contemplated). In some embodiments, SEQ ID NO:X (or fragment thereof, or concatenated fragments thereof) is modified at its N- and/or C-terminal ends (e.g. amidation/esterification of the C-terminus, and/or acylation [e.g. acetylation] of the N-terminus [e.g. fatty acid {or derivative thereof} conjugated/acylated to N-terminus, optionally/preferably wherein the fatty acid moiety comprises/contains between 2 to 100 {or 2 to 25} carbon atoms, optionally being a myristoyl/palmitoyl/stearoyl group]) and/or is esterified (e.g. with a group[s] for esterification disclosed herein) at one or more of its carboxyl groups, wherein X can be 1, or the number of sequences in the Sequence Listing component to this application, or any integer between 1 and the total number of sequences in the Sequence Listing component to this application;
in some particular embodiments, fragment of SEQ ID NO:X is SEQ ID NO:X without its (if applicable) epitope/affinity tag component and/or (if applicable) Cell Penetrating Peptide (CPP) sequence component;
in some particular embodiments, fragment of SEQ ID NO:X is SEQ ID NO:X without its (if applicable) epitope/affinity tag component, and a fatty acid moiety (non-limiting e.g. myristoyl/palmitoyl/stearoyl group) is acylated to its N-terminus, wherein a Cell Penetrating Peptide (CPP) sequence is the most N-terminal component.

First 25 residues (or other residues range) of SEQ ID NO:f (or partially/completely retroinverse sequence thereof) concatenated to SEQ ID NO:e (or a sub-sequence thereof, or concatenated sub-sequences thereof; optionally partially/completely retroinverse), wherein f is an integer selected from the range 1-123 (or the more preferred range 5-123), and wherein e is an integer selected from the range 1-123 (or the more preferred range 5-123); optionally wherein SEQ ID NO:q (or a sub-sequence thereof, or concatenated sub-sequences thereof; optionally partially/completely retroinverse) is concatenated to the N-terminal end of SEQ ID NO:f in this fusion protein, wherein q is an integer selected from the range 124-126 (or from the range 440-638), [different values of f and/or e and/or q are different embodiments; within their aforementioned range constraints, all possible combinations of f, e, q integer values are contemplated]; wherein a lipidic/lipid (e.g. fatty acid or derivative thereof) moiety is acylated to the N-terminal end of this fusion protein (optionally/preferably wherein the fatty acid moiety comprises/contains between 2 to 100 {or 2 to 25} carbon atoms, optionally being a myristoyl/palmitoyl/stearoyl group) and/or one or more of its carboxyl groups are esterified (e.g. with a group[s] for esterification disclosed herein);
optionally/preferably wherein the SEQ ID NO:e component is a fragment thereof, optionally/preferably being one of its following fragments (different fragments in different embodiments): its residues: u−o, wherein u is equal to or an integer greater than 26, o is equal to or an integer less than its total number of residues, and u>o, wherein to illustrate with some non-limiting examples: residues: 26-109, 27-109, 28-109, 29-109, 30-109, 31-109, 32-109, 33-109, 34-109, 35-109, 36-109, 37-109, 38-109, 39-109, 40-109, 41-109, 42-109, 43-109, 44-109, 45-109, 46-109, 47-109, 48-109, 49-109, 50-109, 51-109, 52-109, 53-109, 54-109, 55-109, 56-109, 57-109, 58-109, 59-109, 60-109, 61-109, 62-109, 63-109, 64-109, 65-109, 66-109, 67-109, 68-109, 69-109, 70-109, 71-109, 72-109, 73-109, 74-109, 75-109, 76-109, 77-109, 78-109, 79-109, 80-109, 81-109, 82-109, 83-109, 84-109, 85-109, 86-109, 87-109, 88-109, 89-109, 90-109, 91-109, 92-109, 93-109, 94-109, 95-109, 96-109, 97-109, 98-109, 99-109, 100-109, 101-109, 102-109, 103-109, 104-109, 105-109, 106-109, 107-109, 108-109, and, for each aforementioned fragment, all possible fragments thereof are also contemplated (e.g. truncated at the C-terminus by a number of residues),
wherein to illustrate with some non-limiting examples: residues: 26-85, 35-85, 35-72, 38-72, 39-72, 39-71, 39-70, 39-69, 39-68, 39-67, 67-83, 73-81, 74-80 (+1-60, 10-60, 10-47, 13-47, 14-47, 14-46, 14-45, 14-44, 14-43, 14-42, 42-58, 48-56, 49-55 of bovine IF1 protein),
   wherein to illustrate with some particular non-limiting examples: (wherein each of the following can optionally be esterified at one or more of their carboxyl groups):
   any of SEQ ID NO:127 to SEQ ID NO:129, or any of SEQ ID NO:394 to SEQ ID NO:438 or [wherein each of the following are without their first 14 residues (so without epitope/affinity tag)] any of SEQ ID NO:146 to SEQ ID NO:155, or any of SEQ ID NO:157 to SEQ ID NO:161, or any of SEQ ID NO:166 to SEQ ID NO:335, wherein a fatty acid (or derivative thereof) moiety is acylated to the N-terminal end of the protein (optionally/preferably wherein the fatty acid moiety comprises/contains between 2 to 100 {or 2 to 25} carbon atoms, optionally being a myristoyl/palmitoyl/stearoyl group);
   where the sequence is completely retroinverse, such that the Cell Penetrating Peptide (CPP) sequence is at the C-instead of the N-terminal end, such as with (for example) SEQ ID NO:338 to SEQ ID NO:393, a fatty amine (or derivative thereof) moiety (e.g. decylamine, CAS: 2016-57-1) is conjugated to the C-terminal end of the peptide/protein instead (Peptide-COOH+NH₂-CₙH₂ₙ₊₁=Peptide-CONH—CₙH₂ₙ₊₁).

### Hydrophobic Amino Acid Residue(s) Added “Upstream” of (N-Terminal to) MIS

In some embodiments, a fusion protein comprises (or consists of): presented in N to C-order: [one or more hydrophobic amino acids, optionally up to 50, preferably less than 20]-[MIS]-[IF1 protein/fragment (or sequence variant thereof)], optionally/preferably wherein one or more of this fusion protein's carboxyl groups are esterified (e.g. with a group[s] for esterification disclosed herein), optionally/preferably wherein one or more parts (or sub-part(s) thereof) are retroinverse (optionally/preferably wherein the MIS component isn't), preferably wherein each of the N-terminal section hydrophobic amino acid(s) have a hydrophobicity equal to, or greater than, phenylalanine, even more preferably with a hydrophobicity equal to, or greater than, cyclohexylalanine, optionally comprising either/both of these amino acids, wherein one or more of the added hydrophobic residues may be non-natural (i.e. not coded for by the genetic code). Optionally, the added hydrophobic amino acid(s) can be independently selected from the group consisting of (wherein for each, if applicable, both the L- and D-forms are contemplated as separate embodiments, even if not explicitly shown) L-phenylalanine, D-phenylalanine, L-tryptophan, D-tryptophan, L-phenylglycine, D-phenylglycine, 3,3-diphenyl-L-alanine, 3,3-diphenyl-D-alanine, L-3-cyclohexylalanine, D-3-cyclohexylalanine, L-3-(1-naphthyl) alanine, D-3-(1-naphthyl) alanine, L-3-(2-naphthyl) alanine, D-3-(2-naphthyl) alanine, L-2-aminooctanoic acid, D-2-aminooctanoic acid, O-Methyl-L-tyrosine, O-Methyl-D-tyrosine, 2,6-dimethyl-L-tyrosine, 2,6-dimethyl-D-tyrosine, L-2,4,6-trimethylphenylalanine, D-2,4,6-trimethylphenylalanine, 2-cyclohexyl-L-glycine, 2-cyclohexyl-D-glycine, 3-benzo[b]thiophen-3-yl-L-alanine, 3-benzo[b]thiophen-3-yl-D-alanine, L-homophenylalanine, D-homophenylalanine, 4-fluoro-L-phenylalanine, 4-fluoro-D-phenylalanine, 4-chloro-L-phenylalanine, 4-chloro-D-phenylalanine, 3,4-difluoro-L-phenylalanine, 3,4-difluoro-D-phenylalanine, wherein all the options can optionally have 1 to 4 substituents, independently selected from alkyl and halogen. In alternative embodiments, in addition, more types of hydrophobic amino acid can also be selected from. For example, other amino acids with one or more of cyclo, diphenyl, naphthyl, or hexyl components. In some embodiments, instead of, or in addition to, one or more hydrophobic amino acid residues being added, one or more cysteine residues, with a cholesterol derivative (cholesterol modified with a cysteine-reactive 2-bromoacetyl moiety) attached to its side-chain, are added “upstream” (N-terminal to) the MIS.

Elegantly, once inside the cell, the esterification moieties are cleaved off by esterases, and once the MIS is cleaved off the IF1 protein/fragment (or sequence variant thereof), which happens in the mitochondrial matrix, the N-terminal string of hydrophobic amino acid resides (and/or cysteine(s) with cholesterol derivative bound) is inherently cleaved off along with it, leaving the IF1 protein/fragment (or sequence variant thereof) unencumbered once it has successfully reached its intracellular destination.

In other embodiments, the N-terminal string of one or more hydrophobic amino acids (and/or cysteine(s) with cholesterol derivative bound) can also comprise one or more positively charged residues (e.g. is a Mitochondrial Penetrating Peptide (MPP) sequence as disclosed elsewhere herein), e.g. arginine, and/or partially/completely comprise one or more amino acids that have a reasonably high log P, which are capable of adopting a positive charge e.g. histidine.

Aliphatic Chain Attached (e.g. Fatty Acid Acylated to N-Terminus)

In some fusion protein embodiments, wherein one or more (or zero) carboxyl groups are esterified (e.g. with a group specified herein), a lipophilic moiety (or plural thereof) is attached to the N- and/or C-terminus, and/or to one or more amino-acid side chains of the sequence, wherein this lipophilic moiety can be an alkyl group (preferably with 2 to 100 carbon atoms, more preferably 2 to 25 carbon atoms), optionally an alkyl group with at least one carboxyl group (e.g. fatty acid), or more thereof (e.g. two carboxyl groups in a fatty di-acid), optionally wherein at least one lipophilic substituent is attached to at least one amino acid residue in the peptide/protein sequence in such a way that:
(i) a carboxyl group of a lipophilic substituent forms an amide bond with an amino group of the amino acid residue, or
(ii) an amino group of a lipophilic substituent forms an amide (or ester) bond with a carboxyl group of the amino acid residue, or
(iii) a lipophilic substituent is attached via a “spacer” (optionally wherein the spacer is an amino acid(s) residue, except Cys, or a dipeptide(s) such as Gly-Lys [in either orientation]), optionally wherein
   (a) an amino group of the peptide/protein forms an amide bond with a carboxylic group of a spacer (e.g. amino acid, e.g. L-γ-glutamic acid), and an amino group of the spacer forms an amide bond with a carboxyl group of a lipophilic substituent, or
   (b) a carboxyl group of the peptide/protein forms an amide bond with an amino group of a spacer (e.g. Asp or Glu or a dipeptide containing one or both) and a carboxyl group of the spacer forms an amide bond with an amino group of a lipophilic substituent,
      in some embodiments the lipophilic moiety is an acyl group of a straight-chain or branched fatty acid. The related teaching of WO98/08871, WO99/43706, and similar, are herein incorporated, in their entirety, by reference, especially the lipophilic moieties they disclose and how to attach them to a peptide/protein.

In some fusion protein embodiments, optionally/preferably wherein one or more (or zero) carboxyl groups are esterified (e.g. with a group specified herein), an aliphatic chain (linear or branched) is connected to the N-terminus, optionally containing >5/10/20/30/40/50/100 carbons (preferably less than 25, and more than 10), optionally wherein one or more amino acids (e.g. 1 to 5 residues, optionally wherein each is glycine and/or a hydrophobic amino acid, wherein this amino acid(s) can be termed a “linker”) may be between the aliphatic chain and the start of the MIS sequence. So, in some embodiments, a fusion protein comprises (or consists of): presented in N to C-order (with the aliphatic chain attached to the N-terminus): [aliphatic chain]-[MIS]-[IF1 protein/fragment (or sequence variant thereof)] or [aliphatic chain]-[linker]-[MIS]-[IF1 protein/fragment (or sequence variant thereof)]. In some embodiments, the N-terminal aliphatic chain is that of a fatty acid acylated to the N-terminus: [fatty acid as acyl attachment to N-terminus]-[MIS]-[IF1 protein/fragment (or sequence variant thereof)] or [fatty acid as acyl attachment to N-terminus]-[linker]-[MIS]-[IF1 protein/fragment (or sequence variant thereof)]. In some embodiments, the fatty acid is palmitic acid, attached by an acyl connection to form a palmitoyl group. In alternative embodiments, instead of, or in addition to, an aliphatic chain/fatty acid connected to the N-terminus, at least one aliphatic chain (linear or branched, containing >5/10/20/30/40/50/100 carbons [preferably less than 25 and more than 10]) is/comprises at least one side-chain of at least one non-natural amino acid (e.g. 2-aminooctadecanoic acid) in the peptide/protein sequence, and/or at least one fatty acid is attached/acylated to at least one amino acid side-chain (e.g. to that of lysine(s) e.g. as in insulin detemir or insulin degludec), which is “upstream” of (N-terminal to) the MIS sequence. In some embodiments, an attached aliphatic chain(s) is courtesy of an attached palmitoyl group(s), which can for example, attach to the N-terminus (e.g. as in palmitoyl pentapeptide-4 [Matrixyl™], U.S. Pat. No. 6,620,419B1, wherein the N-terminus is acylated with a palmitoyl group), courtesy of reaction with palmitic acid. In other embodiments, an attached (e.g. to N-terminus) aliphatic chain(s) is courtesy of a different fatty acid group(s) attached, optionally with 2 to 100 (or 2 to 25) carbons, hydroxylated or not, saturated or not, linear or branched, sulfurated or not, cyclic or not. At least one conjugated fatty acid moiety (non-limiting e.g. to one or more of N-terminus, lysine/cysteine/serine/tyrosine side-chain[s]) can confer peptide/protein ability to self-associate and/or bind albumin in the blood, which can (sterically) decrease protease(s) access to the peptide/protein, and/or slow its renal clearance, and dramatically increase its half-life in the blood (e.g. from minutes to hours), which can be extremely advantageous. Conjugation of a fatty acid(s) reported/predicted to bind albumin is especially preferred, and/or conjugation of a fatty acid (and optionally its “spacer” moiety if applicable) present in a licensed/clinical candidate peptide/protein based drug, e.g. in one or more of insulin detemir, insulin degludec, liraglutide, semaglutide. Conjugation of a fatty acid(s), e.g. conjugation with palmitic acid (palmitoylation), can increase skin penetration of a peptide/protein.

SEQUENCE A (not in Sequence Listing because it cannot be sufficiently described by the WIPO.25 standard) comprises up to 25 natural and/or non-natural (e.g. D-amino acid) amino acid residues, preferably less than 10, more preferably less than 5, optionally just 1 amino acid residue long, wherein each and every residue position can be absent (if all are absent, then this sequence is absent), or independently be any amino acid, optionally glycine or alanine, optionally including one or more hydrophobic amino acids (optionally at least as hydrophobic as phenylalanine), optionally including one or more positively charged amino acids (optionally lysine and/or arginine), optionally including one or more reasonably hydrophobic amino acids with, or with an ability to adopt, a positive charge (e.g. histidine), optionally wherein one or more residues are lipidated (covalently bound by a lipidic/lipid moiety [non-limiting e.g. myristate {e.g. bound to lysine}, palmitate {e.g. attached through a thioether bond to a cysteine residue or through an ester bond to a serine or threonine residue}]), optionally wherein there is a fatty acid moiety (saturated or non-saturated, linear or branched; or derivative thereof) conjugated/acylated to this sequence's N-terminus and/or at least one of the amino acid residues has a fatty acid moiety (saturated or non-saturated, linear or branched; or derivative thereof) conjugated to its side-chain, optionally wherein at least one of the amino acid residues is a lysine with a fatty acid moiety joined/acylated to its side-chain (non-limiting examples: optionally connected directly as observed in insulin detemir, alternatively wherein the fatty acid is conjugated to the side-chain via a “spacer” moiety, such as L-γ-glutamic acid, which is used as a spacer [between fatty acid and lysine side-chain] in insulin degludec and liraglutide, wherein the use of a more elaborate spacer [e.g. as in semaglutide, wherein the spacer is L-γ-glutamic acid and two OEG {8-amino-3,6-dioxaoctanoic acid} units] is also contemplated), preferably wherein each conjugated fatty acid contains between 2 and 200 carbons, more preferably between 2 and 25 carbons, optionally being a myristoyl/palmitoyl/stearoyl group, preferably wherein there is only one fatty acid conjugated to this sequence in total. Such a sequence, comprising a lysine residue with a fatty acid conjugated (directly, without a “spacer”) to its side-chain, can be made according to the teaching of (or a shorter sequence can be made by the same teaching, optionally wherein the favouring of negative amino acid residues therein, specifically serine, surrounding the lysine residue, isn't followed [because they can disfavour cellular penetration]), which can then be concatenated to the N-terminal end of a peptide/protein of/in this disclosure, to produce a further peptide/protein of this disclosure. In some embodiments, SEQUENCE A contains at least one cysteine residue with a cholesterol derivative conjugated to its side-chain (e.g. cholesterol modified with a cysteine-reactive 2-bromoacetyl moiety conjugated to cysteine side-chain by a disulphide bond) or contains at least one cysteine residue with a fatty acid (or derivative thereof) moiety conjugated to its side-chain, optionally via a disulphide bond, optionally as taught by the Reversible Aqueous Lipidization (REAL) technology ([196], U.S. Pat. Nos. 5,907,030, 6,093,692, 6,225,445B1, U.S. Pat. No. 7,052,704B2, US2013/0053433A1, WO96/22773), optionally wherein the fatty acid derivative is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated. Note that disulphide bonds break in the reducing intracellular environment. In some preferred embodiments, SEQUENCE A only has a single lipophilic moiety (cholesterol/fatty acid or derivative thereof) bound to it.

SEQUENCE A concatenated to any other sequence of/in this disclosure is, in turn, a sequence of this disclosure. In preferred fusion protein embodiments: (i) if a Mitochondrial Import Sequence (MIS) is present, SEQUENCE A is present “upstream” of (N-terminal to) it [such that it is also cleaved off when the MIS is cleaved off in the mitochondrial matrix], (ii) if an MIS and a CPP sequence are present, SEQUENCE A is present “upstream” of (N-terminal to) the MIS, between the CPP and MIS, or “upstream” of (N-terminal to) both.

SEQ ID NO:439 is an approximation to SEQUENCE A within the constraints of the WIPO.25 standard.

SEQUENCE A or SEQ ID NO:439 concatenated to SEQ ID NO:X (or fragment thereof, or concatenated fragments thereof),
wherein X can be 1, or the number of sequences in the Sequence Listing component to this application, or any integer between 1 and the total number of sequences in the Sequence Listing component to this application.

### Polynucleotides, Vectors, Gene Therapies, Cells, Transgenic Organisms Thereof

A polynucleotide, or a pharmaceutical/cosmetic composition thereof, encoding (by the genetic code; optionally using the codon bias of at least one species that will/might express the polynucleotide, wherein the codons used for each amino acid are the most [or one of the most] frequently used for each amino acid in the codon bias of the species [use instead of a pooled codon bias, of a number of different species that will/might express the polynucleotide, is also contemplated]) at least one peptide/protein of/in this disclosure is a polynucleotide of this disclosure;
A polynucleotide, or a pharmaceutical/cosmetic composition thereof, comprising (or consisting of) at least one nucleotide/nucleic acid sequence that codes/encodes (by the genetic code) for at least one of SEQ ID NO:X (or a fragment thereof, or concatenated fragments thereof);
A polynucleotide, or a pharmaceutical/cosmetic composition thereof, comprising (or consisting of) at least one nucleotide/nucleic acid sequence that codes/encodes (by the genetic code) for at least one of SEQ ID NO:X (or a fragment thereof, or concatenated fragments thereof) and/or that codes/encodes for a sequence variant/conservatively modified variant/functional variant/[conservatively modified, and functional, sequence variant] thereof;
Both the coding and the complimentary non-coding polynucleotide strands are contemplated, existing either single- or double-stranded (or mixture thereof);
A vector/plasmid/gene therapy/virus/cell/transgenic organism, or a pharmaceutical/cosmetic composition thereof, comprising at least one nucleotide/nucleic acid sequence that codes/encodes (by the genetic code) for at least one of SEQ ID NO:X (or a fragment thereof, or concatenated fragments thereof);
A vector/plasmid/gene therapy/virus/cell/transgenic organism, or a pharmaceutical/cosmetic composition thereof, comprising at least one nucleotide/nucleic acid sequence that codes/encodes (by the genetic code) for at least one of SEQ ID NO:X (or a fragment thereof, or concatenated fragments thereof) and/or that codes/encodes (by the genetic code) for a sequence variant/conservatively modified variant/functional variant/[conservatively modified, and functional, sequence variant] thereof;
wherein X can, independently selected in each case of using X, be 1, or the number of sequences in the Sequence Listing component to this application, or any integer between 1 and the total number of sequences in the Sequence Listing component to this application.

Due to the degeneracy/redundancy of the genetic code, it is clear to one of the art how any nucleic acid sequence of/in this disclosure can be modified without modifying the amino acid sequence it codes for by the genetic code, to arrive at further nucleotide sequences of this disclosure. Herein contemplated are polynucleotide variants with one or more codons substituted for a different codon, independently selected at each position, but wherein each substituted codon confers the same amino acid at the same position in the encoded amino acid sequence (a “silent change”). Some other contemplated polynucleotide variants are those conferring a change in amino acid at one or more positions, but wherein each is a “conservative” substitution (substituted amino acid is different but has similar biophysical properties). With the polynucleotide sequences of/in this disclosure in hand, one of the art can arrive at “conservatively modified” variants thereof, which are componentry to this disclosure.

### Section Optimized for Protection in China (Especially, but not Exclusively)

A peptide/protein of (a) or (b) as follows:
(a) a peptide/protein whose amino acid sequence is represented by SEQ ID NO:X (or fragment thereof, or concatenated fragments thereof),
(b) a peptide/protein derived from the peptide/protein of (a) by substitution and/or deletion and/or addition of a number of (e.g. several) amino acids in the amino acid sequence in (a), and having the activity of (a),
wherein peptide/protein (a) or (b) can be synthesized by standard methods of the art (e.g. refer herein, and in references herein) and the activity of peptide/protein (a) or (b) can be verified by a Sub-Mitochondrial Particle (SMP) F₁F₀ ATP hydrolysis assay of the art, as herein described (and in references herein, wherein the exerted activity of (a) and (b) is reduced F₁F₀ ATP hydrolysis at pH 6 and/or pH 8);
wherein X can, independently selected in each case of using X, be 1, or the number of sequences in the Sequence Listing component to this application, or any integer between 1 and the total number of sequences in the Sequence Listing component to this application;
wherein a gene/polynucleotide encoding peptide/protein of (a) or (b) is componentry to this disclosure.

### Description Component Written as a Claim Set

### Cosmetic Thereof

[1] A cosmetic/pharmaceutical/dermopharmaceutical/topical composition comprising (or consisting of) at least one compound that can inhibit/reduce F₁F₀ ATP hydrolysis (e.g. in a cell and/or in a Sub-Mitochondrial Particle [SMP] assay of F₁F₀ ATP hydrolysis), optionally such a compound(s) of this disclosure, optionally at least one compound of at least one of Formula (I), (II), (III), (IV), (V), (VI), (VII), (VIII), [X], and/or at least one cosmetically/pharmaceutically acceptable salt, solvate, hydrate, prodrug, liposome, nanoparticle (e.g. lipid nanoparticle, LNP) or other vector of the art thereof;
optionally in a form suitable for application to the skin/scalp of a subject (optionally human), optionally their face;
optionally wherein the composition contains at least one cosmetically/dermatologically acceptable carrier;
optionally wherein the composition contains at least one further active agent, which can perform one or more of skin/scalp care/protection/treatment/repair/beautification/cleaning/fragrance/cosmetic purpose/appearance change, optionally for human skin/scalp, wherein a number of such agents are known in the art (non-limiting e.g. retinol and other retinoids);
optionally wherein at least one aim/use of the cosmetic/pharmaceutical/dermopharmaceutical/topical composition (non-limiting e.g. as communicated in text and/or visually and/or diagrammatically and/or verbally and/or electronically in one or more of its packaging, associated packaging, package/paper insert(s), instruction(s) and/or warning(s) for use, marketing, promotion, advertisement(s), product education, associated website[s]) is changing/improving the appearance/aesthetic of the administered subject, optionally for conferring/maintaining a younger appearance.

[2] A cosmetic/pharmaceutical/dermopharmaceutical/topical composition comprising (or consisting of) at least one [any] IF1 protein/fragment (or sequence variant(s) thereof, including partially/completely retroinverse sequence thereof), and/or fusion protein(s) thereof (optionally comprising at least one Cell Penetrating Peptide [CPP] sequence, optionally a Tat and/or poly-arginine sequence, optionally partially/completely retroinverse sequence thereof), optionally lipidated (i.e. with at least one covalently bound lipidic/lipid moiety, optionally at least one fatty acid [optionally acylated to its N-terminus] e.g. [non-limiting] a myristoyl/palmitoyl/stearoyl group), optionally modified at its N- (non-limiting e.g. acylated [non-limiting e.g. acetylated]) and/or C-terminal (non-limiting e.g. amidated) ends, optionally containing one or more non-natural amino acids, optionally wherein one or more amino-acids in the sequence are corresponding D-amino acids, optionally wherein one or more of its carboxyl groups are esterified, and/or at least one cosmetically/pharmaceutically acceptable salt, solvate, hydrate, prodrug, liposome, nanoparticle (e.g. lipid nanoparticle, LNP) or other vector of the art thereof;
optionally a cosmetic composition comprising (or consisting of) at least one peptide/protein (optionally with one or more of its carboxyl groups esterified) comprising (or consisting of) [preferably wherein the following is in N to C-terminal order] at least one Cell Penetrating Peptide sequence (CPP, e.g. a poly-arginine CPP, optionally with a fatty acid [e.g. of between 2 to 25 carbons] acylated to its N-terminal end) conjoined with (e.g. peptide bonded to) at least one Mitochondrial Import Sequence (MIS; conferring mitochondrial matrix localization, optionally/preferably wherein the MIS is that used by the species administered to for its native IF1 protein; e.g. MIS that human uses for its native IF1 protein) conjoined with (e.g. peptide bonded to) at least one “mature” (without MIS) IF1 protein/fragment (or sequence variant thereof) that is optionally/preferably an IF1 protein sufficiently truncated at its C-terminal end (non-limiting e.g. truncated up to {using “mature” [without MIS] IF1 protein numbering} its 60^{th} or 47^{th} residue), and/or optionally truncated at its N-terminal end (non-limiting e.g. by any number of residues up to 9 [or 13] residues), and/or with one or more amino acid substitutions in its “phosphorylation control switch” and/or “pH dependence motif” (FIG. 10; non-limiting e.g. one or more of {using “mature” [without MIS] IF1 protein numbering} S14A [or T14A], E26A [or Q26A or E26Q], H48A [or Y48A], H49K [or H49A or H49R], H55A [or Y55A or V55A], H56A [or T56A or S56A] substitutions), such that it can still inhibit F₁F₀ ATP hydrolysis but it cannot (or cannot as readily) form IF1 protein tetramers (and higher oligomers) at alkaline pH, preferably such that it can more potently inhibit F₁F₀ ATP hydrolysis at the normal, alkaline pH (˜pH 8) of the mitochondrial matrix (than native/unmodified IF1 protein), optionally/preferably wherein this IF1 protein/fragment (or sequence variant thereof) has a sequence derived/modified from the IF1 protein sequence of the species to be administered to [e.g. human] or a species with a longer maximal lifespan, optionally a species with a very long maximal lifespan such as a whale, e.g. bowhead or blue whale, and/or at least one cosmetically acceptable salt, solvate, hydrate, prodrug, liposome, nanoparticle (e.g. lipid nanoparticle, LNP) or other vector of the art thereof;
optionally a cosmetic composition comprising (or consisting of) at least one peptide/protein comprising (or consisting of) at least one sequence that is a designed concatenation of sequences that are each naturally occurring in the human body, e.g, wherein the CPP component is SEQ ID NO:455 (or SEQ ID NO:461, or residues 4-11 of SEQ ID NO:453), the MIS component is SEQ ID NO:162, and the IF1 protein/fragment sequence component is from human IF1 protein (non-limiting e.g. using “mature” [without MIS] IF1 protein numbering: residues: 1-60, 10-60, 14-60, 13-47, 14-47, 42-58, which are the amino acid sequences encoded by DNA sequences SEQ ID NO:1473, SEQ ID NO:1476, SEQ ID NO:1479, SEQ ID NO:1482, SEQ ID NO:1485, SEQ ID NO:1488 respectively), and/or at least one cosmetically acceptable salt, solvate, hydrate, prodrug, liposome, nanoparticle (e.g. lipid nanoparticle, LNP) or other vector of the art thereof;
optionally a cosmetic/pharmaceutical/dermopharmaceutical/topical composition comprising (or consisting of) at least one peptide/protein comprising (or consisting of) at least one of SEQ ID NO:X (or fragment thereof or concatenated fragments thereof and/or sequence variant thereof), wherein X can be 1, or the number of sequences in the Sequence Listing component to this application, or any integer between 1 and the total number of sequences in the Sequence Listing component to this application,
[e.g. optionally a cosmetic/pharmaceutical/dermopharmaceutical/topical composition comprising (or consisting of) at least one peptide/protein comprising (or consisting of) at least one sequence selected from SEQ ID NO:166 to SEQ ID NO:438, and/or at least one fragment thereof (for non-limiting example wherein the epitope/affinity tag component [if present] is absent, and/or the Cell Penetrating Peptide component [if present] is absent), and/or concatenated fragments thereof, and/or a functional (can inhibit/reduce F₁F₀ ATP hydrolysis in a cell and/or in a Sub-Mitochondrial Particle [SMP] assay of F₁F₀ ATP hydrolysis) sequence variant(s) thereof {optionally produced by conservative substitution(s)}], and/or at least one cosmetically/pharmaceutically acceptable salt, solvate, hydrate, prodrug, liposome, nanoparticle (e.g. lipid nanoparticle, LNP) or other vector of the art thereof;
optionally in a form suitable for application to the skin/scalp of a subject (optionally human), optionally their face;
optionally wherein the composition contains at least one cosmetically/dermatologically acceptable carrier;
optionally wherein the composition contains at least one further active agent, which can perform one or more of skin/scalp care/protection/treatment/repair/beautification/cleaning/fragrance/cosmetic purpose/appearance change, optionally for human skin/scalp, wherein a number of such agents are known in the art (e.g. retinol and other retinoids);
optionally wherein at least one aim/use of the cosmetic/pharmaceutical/dermopharmaceutical/topical composition (non-limiting e.g. as communicated in text and/or visually and/or diagrammatically and/or verbally and/or electronically in one or more of its packaging, associated packaging, package/paper insert(s), instruction(s) and/or warning(s) for use, marketing, promotion, advertisement(s), product education, associated website[s]) is changing/improving the appearance/aesthetic of the administered subject, optionally for conferring/maintaining a younger appearance.

[3] A cosmetic/pharmaceutical/dermopharmaceutical/topical composition comprising (or consisting of) at least one peptide derived from at least one IF1 protein.

[4] A cosmetic/pharmaceutical/dermopharmaceutical/topical composition according to one or more of Claims 1-3 wherein at least one IF1 protein/fragment (or sequence variant thereof) is present at a concentration between 0.000001% [or 0.0001%] (w/w) and 5% [or 15% or 20% or 70%] (w/w) by total weight of the composition.

[5] A cosmetic/pharmaceutical/dermopharmaceutical/topical composition according to one or more of Claims 1-4 further comprising one or more ingredients selected from a group comprising (or consisting of):
extracted lipids, synthetic lipids, gelifying polymers, thickening polymers, tensioactive polymers, emulsifying polymers, hydrosoluble active principles, liposoluble active principles, plant extracts, vegetable extracts, tissue extracts, marine extracts, cell extracts, essential oils, mineral salts, solar/sun filters, antioxidants, organic or aqueous-glycolic solvents, fatty substances, ionic or non-ionic thickeners, softeners, opacifiers, stabilizers, emollients, silicones, alpha-hydroxy acids, antifoaming agents, moisturizing agents, vitamins, perfumes, preservatives, sequestrating agents, colouring agents, gel-forming and viscosity increasing polymers, surfactants and emulsifiers, other water- or fat-soluble active substances, moisturizers, barrier agents, skin-revitalizing agents, organic solvents,
an amount (preferably a cosmetically/pharmaceutically effective amount) of at least one active agent selected from a group comprising (or consisting of): retinol, retinoid, agents which inhibit PAR-2 activity, analgesic agents, anesthetic agents, cyclic adenosine monophosphate synthesis stimulating agents, elastase inhibiting agents, matrix metalloproteinases inhibiting agents, melanin synthesis stimulating or inhibiting agents, whitening or depigmenting agents, propigmenting agents, self-tanning agents, anti-aging agents, NO-synthase inhibiting agents, 5α-reductase inhibiting agents, lysyl- and/or prolyl hydroxylase inhibiting agents, antioxidants, free radical scavengers, agents against atmospheric pollution, reactive carbonyl species scavengers, anti-glycation agents, antihistamine agents, anti-viral agents, anti-parasitic agents, emulsifiers, emollients, organic solvents, liquid propellants, skin and/or hair conditioners, humectants, substances that retain moisture, alpha hydroxyacids, beta hydroxyacids, moisturizers, epidermal hydrolytic enzymes, vitamins, pigments or colorants, dyes, gelling polymers, thickeners, surfactants, softening agents, anti-wrinkle agents, agents able to reduce or treat the bags under the eyes, exfoliating agents, antimicrobial agents, anti-fungal agents, fungistatic agents, bactericidal agents, bacteriostatic agents, agents stimulating the synthesis of dermal or epidermal macromolecules and/or capable of inhibiting or preventing their degradation, collagen synthesis-stimulating agents, elastin synthesis-stimulating agents, decorin synthesis-stimulating agents, laminin synthesis-stimulating agents, defensin synthesis-stimulating agents, cAMP synthesis-stimulating agents, chaperone synthesis stimulating agents, aquaporin synthesis-stimulating agents, hyaluronic acid synthesis-stimulating agents, fibronectin synthesis-stimulating agents, sirtuin synthesis-stimulating agents, agents stimulating the synthesis of lipids and components of the stratum corneum, agents stimulating the synthesis of ceramides, ceramides, agents that inhibit collagen degradation, agents that inhibit elastin degradation, agents that inhibit serine proteases such us cathepsin G, agents stimulating fibroblast proliferation, agents stimulating keratinocyte proliferation, agents stimulating adipocyte proliferation, agents stimulating melanocyte proliferation, agents stimulating keratinocyte differentiation, agents stimulating adipocyte differentiation, agents that inhibit acetylcholinesterase, skin relaxant agents, agents that inhibit acetylcholine receptors aggregation, agents that inhibit muscle contraction, anticholinergic agents, glycosaminoglycan synthesis-stimulating agents, antihyperkeratosis agents, comedolytic agents, antipsoriasis agents, DNA repair agents, DNA protecting agents, stabilizers, anti-itching agents, agents for the treatment and/or care of sensitive skin, firming agents, anti-stretch mark agents, binding agents, agents regulating sebum production, lipolytic agents or agents stimulating lipolysis, anti-cellulite agents, agents modulating the activity of PPARY, agents which increase or reduce the triglyceride content of adipocytes, antiperspirant agents, agents stimulating healing, coadjuvant healing agents, agents stimulating reepithelialization, coadjuvant reepithelialization agents, cytokine growth factors, calming agents, anti-inflammatory agents, anesthetic agents, agents acting on capillary circulation and/or microcirculation, agents stimulating angiogenesis, agents that inhibit vascular permeability, venotonic agents, agents acting on cell metabolism, agents to improve dermal-epidermal junction, agents inducing hair growth, hair growth inhibiting or retardant agents, agents preventing/delaying hair loss, preservatives, perfumes, chelating agents, agents obtained from a biofermentation process, agents obtained from a biotechnological process, mineral salts, sunscreens, organic or mineral photoprotective agents active against ultraviolet A and/or B rays and/or infrared A rays, amino acids, peptides, proteins, enzymes, catalysts, cleaning agent, hair conditioning agent, skin conditioning agent, hair styling agent, antidandruff agent, hair growth promoter, perfume, sunscreen, sunblock, pigment, moisturizer, film former, hair colour, make-up agent, detergent, pharmaceutical, thickening agent, emulsifier, humectant, emollient, antiseptic agent, deodorant active, dermatologically acceptable carrier, surfactant, abrasive, absorbent, fragrance, colouring/colorant, essential oil, skin sensate, astringent, anti-acne agent, anti-caking agent, antifoaming agent, antimicrobial, antioxidant, binder, biological additive, enzyme, enzyme inhibitor, enzyme activator, coenzyme, botanical extract, ceramide, peptide, buffering agent, bulking agent, chelating agent, cosmetic biocide, polymer, quat, substantivity increasing agent, opacifying agent, pH adjuster, propellant, reducing agent, sequestrant, skin bleaching and/or lightening agent, skin conditioning agent, skin soothing and/or healing agent, aloe vera, pantothenic acid and/or derivative thereof, allantoin, bisabolol, dipotassium glycyrrhizinate, skin treating agent, thickener, and/or mixtures thereof.

[6] A cosmetic/pharmaceutical/dermopharmaceutical/topical composition according to one or more of Claims 1-5 wherein one or more active agents are encompassed/incorporated in one or more of carriers, liposomes, mixed liposomes, lipid particles, lipid vesicles, oleosomes, niosomes, ethosomes, millicapsules, microcapsules, nanocapsules, sponges, microsponges, cyclodextrins, vesicles, micelles, mixed micelles of surfactants, mixed micelles of surfactant-phospholipid, millispheres, microspheres, nanospheres, lipospheres, microemulsions, nanoemulsions, miniparticles, milliparticles, microparticles, nanoparticles (Lipid NanoParticles, LNPs), solid lipid nanoparticles, nanostructured lipid carriers, phospholipid mini-vesicles, or similar, or a carrier used in one or more cosmetics/dermopharmaceuticals/pharmaceuticals on the market and/or in clinical trials.

[7] A cosmetic/pharmaceutical/dermopharmaceutical/topical composition according to one or more of Claims 1-6 in at least one form/formulation selected from a group comprising (or consisting of): gel, emulsion, oil/water emulsion, water/oil emulsion, milk, lotion, ointment, stick, pencil, spray, cream, cream gel, multiple emulsion, anhydrous composition, aqueous dispersion, oil, balsam, foam, hydroalcoholic solution, hydroglycolic solution, hydrogel, liniment, sera, serum, mousse, pomade, powder, bar, aerosol, granule, solution, suspension, emulsion, syrup, polysaccharide film, jelly, gelatin, emollient lotion, emollient milk, emollient cream, emulsion of oil and/or silicone in water, emulsion of water in oil and/or silicone, balm, liquid, paste, aerosol, butter,
and/or incorporated into a product for administration/application to the human skin/scalp, optionally selected from a group comprising (or consisting of): cosmetic product for use upon the skin/scalp, cosmetic product for use upon the face, daily use skin care product, exfoliant, skin smoothing product, product for improving/smoothing skin texture, anti-aging/anti-wrinkle skin product/cream/serum, anti-hair loss product, hair growth promoting product, anti-hair greying product, hair dye, skin cream, face cream, eye cream, anti-acne/spot cream, moisterizer, cleanser, shampoo, conditioner, anti-dandruff product, soap, shower gel, scalp lotion, body oil, skin/body/face scrub, milk/cream for care of skin and/or hair, cleansing cream, foundation tint base, sunscreen/sunblock/sun cream (e.g. offering protection against UVA and/or UVB radiation), fake sun tan product, skin darkening product, skin whitening product/cream, shaving cream/foam/balm, perfume, aftershave, deodorant, anti-persperant, make-up product, lip rouge, lipstick, lip gloss, lip protector, mascara, nail varnish, concealer, under-eye concealer, blusher, mascara, make-up foundation, foundation, BB cream or CC cream or DD cream or similar, make-up removing product/lotion/milk/cream, eye shadow, unguent, anti-cellulite product, anti-stretch mark product, anti-varicose vein product, daily peel, face mask, eye mask, night mask, sleeping mask, toothpaste, mouthwash,
and/or incorporated/absorbed/adsorbed into one or more of a fabric, non-woven fabric, textile, a material used for clothing, garment, natural or synthetic fibre, wool, face mask, sleeping mask, eye mask, plaster, medical device, bandage, gauze, wipe, patch, adhesive skin patch, non-adhesive skin patch, microelectric patch, towelette, hydrogel,
and/or adsorbed on at least one cosmetically/pharmaceutically acceptable solid organic polymer or solid mineral support selected from the group comprising (or consisting of) talc, bentonite, silica, starch, maltodextrin or inorganic carrier, adsorbed on powdered organic and/or inorganic polymers.

[8] A method that comprises administering to a subject (and/or the subject self-administering) an amount (preferably an effective amount e.g. a cosmetically effective amount) of at least one cosmetic/pharmaceutical/dermopharmaceutical/topical composition according to one or more of claims 1-7;
optionally administered to the subject's skin/scalp, optionally their face, optionally wherein the subject is a human.

[9] A method of eliminating/reducing/slowing/delaying/preventing one or more visible signs of aging comprising applying at least one cosmetic/pharmaceutical/dermopharmaceutical/topical composition according to one or more of Claims 1-7 to one or more areas of human skin (optionally already showing one or more signs of aging), preferably wherein said composition is applied at least once per day for a time period sufficient to provide an elimination/reduction/slowing/delay/prevention in the visible signs of aging of that portion of human skin, wherein said time period is at least 2 weeks (optionally wherein the administered region of human skin is compared to an unadminstered region of human skin, optionally of same/similar age, optionally from the same person, to compare their degree/severity of one or more visable signs of aging).

A method of treatment and/or care of the skin which comprises administering to the skin an amount (preferably an effective amount) of at least one cosmetic/pharmaceutical/dermopharmaceutical/topical composition according to one or more of Claims 1-7,
optionally wherein said treatment and/or care of the skin is the treatment of one or more signs of aging and/or photoaging and/or treatment of skin affected by wrinkles and/or expression lines.

### New Compositions of Matter

A novel functional (can inhibit/reduce F₁F₀ ATP hydrolysis [e.g. in a Sub-Mitochondrial Particle (SMP) assay of F₁F₀ ATP hydrolysis]) IF1 protein/fragment comprising (or consisting of) married/combined fragment(s)/residue(s) of IF1 protein sequences from two or more different species, preferably wherein at least one of these species has a high maximal lifespan, preferably equal to or longer than human, more preferably longer than human e.g. bowhead or blue whale;
optionally wherein the IF1 protein sequence of a species has a C-terminal region (e.g. containing the “pH dependence motif” [FIG. 10]) substituted with the corresponding region (same range of residue position numbers) from a different species, preferably a species with a greater maximal lifespan, e.g. human IF1 protein sequence (optionally containing a S14A substitution) with a C-terminal region substituted with the corresponding sequence region (same range of residue position numbers) from blue or bowhead whale IF1 protein, non-limiting e.g, wherein (using “mature” [without MIS] IF1 protein numbering) up to the 42^{nd} or 47^{th} residue is human IF1 protein sequence, and the remainder of the sequence is from blue or bowhead whale IF1 protein sequence (or sequence variant thereof, e.g. containing a H49K substitution);
non-limiting e.g. sequence wherein (using “mature” [without MIS] IF1 protein numbering) up to its 42^{nd} or 47^{th} residue is blue whale IF1 protein sequence (optionally with a T14A substitution), and the remainder of the sequence is from bowhead whale IF1 protein sequence (or sequence variant thereof, e.g. containing a H49K substitution), optionally a fusion protein thereof (e.g. of a form used herein, e.g. comprising one or more of an epitope/affinity tag, CPP, MIS sequence);
in some embodiments the N-terminal region (non-limiting e.g. up to the 42^{nd} or 47^{th} residue, using “mature” [without MIS] IF1 protein numbering) of a novel IF1 protein/fragment (or sequence variant thereof) is from a species of which one or more subjects will be administered with it (and/or administered with a fusion protein thereof; optionally for at least one therapeutic/cosmetic purpose disclosed herein; non-limiting e.g. human or mouse) and part or all of the remainder/C-terminal region (non-limiting e.g. comprising part or all of the “pH dependence motif” [FIG. 10], and/or [using “mature” {without MIS} IF1 protein numbering] up to the 60^{th} or 81^{st} or 82^{nd} [if possible] or 84^{th} [if possible] residue of the IF1 protein) is from the IF1 protein sequence (or sequence variant thereof, optionally with a H49K substitution) of a longer-living species, with a higher maximal lifespan (non-limiting e.g. blue or bowhead whale).

An “immature” (with Mitochondrial Import Sequence, MIS) or “mature” (without MIS) IF1 protein/fragment (or sequence variant thereof), or fusion protein thereof, wherein one or more of the following applies to (is true of) part(s) or all of it (wherein all possible combinations are contemplated {including all possible combinations of elements/descriptors within, and across, different bullet points} except those that are mutually exclusive):
(i) produced/isolated/purified/substantially purified/partially purified;
(ii) associated with a pharmaceutically/cosmetically acceptable salt[s];
(iii) IF1 protein/fragment (or sequence variant thereof) component/entirety comprises (or consists of) bowhead whale (Balaena mysticetus) IF1 protein;
(iv) IF1 protein/fragment (or sequence variant thereof) component/entirety comprises (or consists of) blue whale (Balaenoptera musculus) IF1 protein;
(v) one or more (or two/three/four/five/six/seven/eight/nine/ten/eleven/twelve/thirteen/fourteen/fifteen/sixteen/seventeen or more) of the following is true of the IF1 protein/fragment (or sequence variant thereof) component/entirety: (using “mature” [without Mitochondrial Import Sequence, MIS] IF1 protein numbering): 49^{th} residue is not histidine, 14^{th} residue is not a residue that can be phosphorylated (i.e. is not serine or threonine), 26^{th} residue is not glutamic acid, 48^{th} residue is not histidine, 55^{th} residue is not histidine, 56^{th} residue is not histidine, 49^{th} residue is lysine or alanine or arginine, 14^{th} residue is alanine, 26^{th} residue is alanine or glutamine, 48^{th} residue is alanine, 55^{th} residue is alanine, 56^{th} residue is alanine, 79^{th} residue is glycine or asparagine, 76^{th} residue is lysine, 73^{rd} residue is serine, 62^{nd} residue is histidine, 82^{nd} residue is aspartic acid, 83^{rd} residue is aspartic acid, 84^{th} residue is aspartic acid, 85^{th} residue is aspartic acid, 57^{th} residue is valine, 54^{th} residue is serine or aspartic acid, 61^{st} residue is glutamine, 51^{st} residue is asparagine, 47^{th} residue is glutamic acid, 46^{th} residue is arginine, 44^{th} residue is serine, 39^{th} residue is lysine, 38^{th} residue is alanine or glutamic acid, 37^{th} residue is arginine or cysteine or lysine, 36^{th} residue is aspartic acid or glutamic acid, 29^{th} residue is histidine, 27^{th} residue is alanine, 25^{th} residue is lysine, 17^{th} residue is aspartic acid, 12^{th} residue is glycine, 11^{th} residue is serine or threonine, 10^{th} residue is serine or glycine, 9^{th} residue is glycine, 8^{th} residue is leucine or glycine, 6^{th} residue is aspartic acid or glycine, 5^{th} residue is alanine, 4^{th} residue is serine or glycine, 3^{rd} residue is glutamic acid or serine or lysine, 2^{nd} residue is glycine, 1^{st} residue is leucine, wherein in particular sub-embodiments (wherein all possible combinations are contemplated, except if mutually exclusive):
   (a) three (3) or more of the list is true;
   (b) five (5) or more of the list is true;
   (c) seven (7) or more of the list is true;
   (d) nine (9) or more of the list is true;
   (e) eleven (11) or more of the list is true;
   (f) thirteen (12) or more of the list is true;
   (g) fourteen (14) or more of the list is true;
   (h) fifteen (15) or more of the list is true;
   (i) sixteen (16) or more of the list is true;
   (j) seventeen (17) or more of the list is true;
   (k) at least one IF1 protein/fragment sequence variant comprises (or consists of) the IF1 protein/fragment sequence of human (or other species with a long maximal lifespan, optionally a species with a longer maximal lifespan than human) with one or more substitutions (and/or addition of one or more aspartic acid residues to its C-terminal end) to make one or more of the list be true;
   (l) at least one IF1 protein/fragment sequence variant comprises (or consists of) the IF1 protein/fragment sequence of bowhead/blue whale with one or more substitutions (and/or addition of one or more aspartic acid residues to its C-terminal end) to make one or more of the list be true;
(vi) IF1 protein/fragment (or sequence variant thereof) component/entirety comprises (or consists of) a sequence variant of IF1 protein (preferably wherein one/two/three/four/five or more of the descriptors in bullet point (v) above apply to it) found in a long-lived species (high maximal lifespan), preferably which has an equal or greater maximal lifespan than Bos taurus, more preferably which has an equal or greater maximal lifespan than human, and more preferably which has a greater maximal lifespan than human e.g. bowhead or blue whale;
(vii) IF1 protein/fragment (or sequence variant thereof) component/entirety comprises (or consists of) an IF1 protein/fragment (optionally from mammal, Bos taurus, or human, or blue or bowhead whale) with one or more of {using “mature” [without MIS] IF1 protein numbering} S14A (or T14A), H49K (or H49A or H49R), E26A (or E26Q or Q26A), H48A (or Y48A), H55A (or Y55A), H56A (or T56A or S56A) substitutions, optionally also/instead with 1-3 (or 1-5) aspartic acid (D) residues added to its C-terminal end;
(viii) IF1 protein/fragment (or sequence variant thereof) component/entirety comprises (or consists of) human IF1 protein/fragment with one or more of {using “mature” [without MIS] IF1 protein numbering} S14A, H49K (or H49A or H49R), E26A (or E26Q), H48A, H55A, H56A substitutions, optionally also/instead with 1-3 (or 1-5) aspartic acid (D) residues added to its C-terminal end;
(ix) IF1 protein/fragment (or sequence variant thereof) component/entirety comprises (or consists of) blue whale IF1 protein/fragment with one or more of {using “mature” [without MIS] IF1 protein numbering} T14A, H49K (or H49A or H49R), E26A (or E26Q), H48A, H55A, H56A substitutions, optionally also/instead with 1-3 (or 1-5) aspartic acid (D) residues added to its C-terminal end;
(x) IF1 protein/fragment (or sequence variant thereof) component/entirety comprises (or consists of) bowhead whale IF1 protein/fragment with one or more of {using “mature” [without MIS] IF1 protein numbering} H49K (or H49A or H49R), E26A (or E26Q), H48A, H55A, H56A substitutions, optionally also/instead with 1-3 (or 1-5) aspartic acid (D) residues added to its C-terminal end;
(xi) IF1 protein/fragment (or sequence variant thereof) component/entirety comprises (or consists of) an IF1 protein fragment (using “mature” [without MIS] IF1 protein numbering) less than z amino acids long, wherein z is an integer selected from a group comprising 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 [different values of z are different embodiments];
(xii) IF1 protein/fragment (or sequence variant thereof) component/entirety comprises (or consists of) an IF1 protein fragment (using “mature” [without MIS] IF1 protein numbering) x-y, where x is an integer between 1 and 20 (or between 1 and 44, or between 1 and 84), and y is an integer between 40 and 85 (or between 40 and 85, or between 2 and 85) [different values of x and/or y are different embodiments; within their aforementioned range constraints, all possible combinations of x and y integer values are contemplated];
(xiii) IF1 protein/fragment (or sequence variant thereof) component/entirety comprises (or consists of) an IF1 protein fragment selected from a group comprising (using “mature” [without MIS] IF1 protein numbering): 1-84, 2-84, 3-84, 4-84, 5-84, 6-84, 7-84, 8-84, 9-84, 10-84, 11-84, 12-84, 13-84, 14-84, 15-84, 16-84, 17-84, 18-84, 19-84, 20-84, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 81-84, 82-84, 83-84, OR a sub-sequence/fragment of one of these aforementioned fragments;
(xiv) IF1 protein/fragment (or sequence variant thereof) component/entirety comprises (or consists of) an IF1 protein fragment selected from a group comprising: IF1 protein residues {using “mature” [without MIS] IF1 protein numbering}: 14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 14-60, 10-84, 14-84, 18-84, 10-50, 1-45, 42-56, 42-47, 48-56, 49-55;
(xv) IF1 protein/fragment (or sequence variant thereof) component/entirety comprises (or consists of) IF1 protein residues [preferably from a species with a maximal lifespan at least as long as Bos taurus, more preferably from a very long-lived mammal e.g. human, or (more preferred) blue or bowhead whale] (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably wherein, if the 14^{th} residue isn't alanine, it is substituted to be alanine;
(xvi) IF1 protein/fragment (or sequence variant thereof) component/entirety comprises (or consists of) human IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably with a S14A substitution;
(xvii) IF1 protein/fragment (or sequence variant thereof) component/entirety comprises (or consists of) whale (non-limiting e.g. selected from a group comprising bowhead, fin, blue, humpback, killer, sperm, gray, Cuvier's beaked, long-finned pilot whale) IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably wherein, if the 14^{th} residue isn't alanine, it is substituted to be so;
(xviii) IF1 protein/fragment (or sequence variant thereof) component/entirety comprises (or consists of) blue whale IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably with a T14A substitution;
(xix) IF1 protein/fragment (or sequence variant thereof) component/entirety comprises (or consists of) bowhead whale IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60;
(xx) IF1 protein/fragment (or sequence variant thereof) component/entirety comprises (or consists of) mammal/Bos Taurus/rodent/mouse/rat/rabbit IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, 42-58, 42-59;
(xxi) IF1 protein/fragment (or sequence variant thereof) component/entirety comprises (or consists of) IF1 protein residues (using “mature” [without MIS] IF1 protein numbering): 42-58 or 42-59 (or sequence variant thereof);
(xxii) IF1 protein/fragment (or sequence variant thereof) component/entirety comprises (or consists of) human IF1 protein residues (using “mature” [without MIS] IF1 protein numbering): 42-58 or 42-59 (or sequence variant thereof), optionally with one or more of E51N, V54S, K57V substitutions;
(xxiii) IF1 protein/fragment (or sequence variant thereof) component/entirety comprises (or consists of) IF1 protein fragment shorter than 40 (or 35, or 30, or 25, or 20, or 15, or 10, or 5) amino acids, which contains H49 residue (or sequence variant thereof);
(xxiv) IF1 protein/fragment (or sequence variant thereof) component/entirety comprises (or consists of) short-living mammal (e.g. rodent, e.g. mouse) IF1 protein fragment shorter than 40 (or 35, or 30, or 25, or 20, or 15, or 10, or 5) amino acids, which contains H49 residue (or sequence variant thereof);
(xxv) IF1 protein/fragment (or sequence variant thereof) component/entirety comprises (or consists of) IF1 protein fragment (or sequence variant thereof) from a species that will be administered with the peptide/protein (and/or composition thereof);
(xxvi) IF1 protein/fragment (or sequence variant thereof) component/entirety comprises (or consists of) IF1 protein fragment (or sequence variant thereof) from a species with a maximal lifespan at least as long as Bos taurus, more preferably from a very long-lived mammal e.g. human, or (more preferred) blue or bowhead whale;
(xxvii) IF1 protein/fragment (or sequence variant thereof) component/entirety comprises (or consists of) IF1 protein fragment (or sequence variant thereof) from a mammal species with a maximal lifespan at least as short as mouse;
(xxviii) IF1 protein/fragment (or sequence variant thereof) component/entirety comprises (or consists of) married/combined fragment(s) and/or residue(s) of IF1 protein sequences from two or more different species, preferably wherein at least one of these species has a high maximal lifespan, preferably equal to or longer than human e.g. human, bowhead or blue whale, more preferably longer than human e.g. bowhead or blue whale;
(xxix) modified at its N- and/or C-terminal ends, optionally amidation/esterification of the C-terminus and/or acylation (e.g. acetylation) of the N-terminus;
(xxx) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) acylated to its N-terminus, non-limiting e.g. myristoyl/palmitoyl/stearoyl group conjugated to N-terminus;
(xxxi) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(xxxii) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated;
(xxxiii) part(s) or all is cyclized, in one or more cycles;
(xxxiv) part(s) or all is bicyclic via attachment to a scaffold(s), optionally rendered bicyclic by judicious insertion of cysteine residues which confer attachment to a scaffold structure by thioether and/or disulphide bonds;
(xxxv) N^{α}-alkylated (e.g. N^{α}-methylated) at one or more places;
(xxxvi) contains one or more corresponding (to its sequence) D-amino acids;
(xxxvii) contains one or more retroinverse regions, or all of it is retroinverse;
(xxxviii) IF1 protein/fragment (or sequence variant thereof) component/entirety is retroinverse;
(xxxix) at least one of its carboxylic groups is esterified, optionally such that one or more of its carboxyl (COOH) groups is replaced by the group (or analogue thereof):

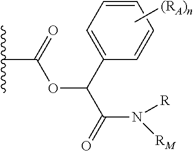

wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example:

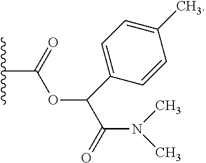 [SMILES: *C(=O)OC(C(=O)N(C)C)c1ccc(C)cc1]

An IF1 protein/fragment (or sequence variant thereof), or fusion protein thereof, according to claim 4 wherein (from a sub-list(s) and or the overall list) x or more aspects/features/descriptors/modifications are true of it, wherein x is an integer selected from the group comprising: 2, 3, 4, 5, 6, 7, 8, 9, 10, 11, 12, 13, 14, 15, 16, 17, 18, 19, 20 [different values of x are different embodiments].

A fusion protein according to Claim 12 comprising (or consisting of):
(i) at least one Mitochondrial Import Sequence (MIS) [for delivery into the mitochondrial matrix], optionally in the order (N-terminal shown first): [MIS]-[IF1 protein/fragment (or sequence variant thereof)]; or
(ii) at least one epitope/affinity tag and at least one MIS [for delivery into the mitochondrial matrix], optionally in the order (N-terminal shown first): [epitope/affinity tag]-[MIS]-[IF1 protein/fragment (or sequence variant thereof)]; or
(iii) at least one MIS [for delivery into the mitochondrial matrix] and at least one Cell Penetrating Peptide (CPP) sequence, optionally in the order (N-terminal shown first): [CPP]-[MIS]-[IF1 protein/fragment (or sequence variant thereof)]; or
(iv) at least one epitope/affinity tag and at least one MIS [for delivery into the mitochondrial matrix] and at least one CPP sequence, optionally in the order (N-terminal shown first): [epitope/affinity tag]-[CPP]-[MIS]-[IF1 protein/fragment (or sequence variant thereof)].

A fusion protein according to Claim 14, wherein one or more of the following applies to (is true of) part(s) or all of it (wherein all possible combinations are contemplated {including all possible combinations of elements/descriptors within, and across, different bullet points} except those that are mutually exclusive):
(i) produced/isolated/purified/substantially purified/partially purified;
(ii) associated with a pharmaceutically/cosmetically acceptable salt[s];
(iii) Mitochondrial Import Sequence (MIS) is the same that a species uses for one or more of its proteins that it transports from the cytoplasm into the mitochondrial matrix, optionally wherein the MIS is the same that a species uses for its native IF1 protein;
(iv) MIS is that for an IF1 protein from human or mouse;
(v) MIS and IF1 protein/fragment (or sequence variant thereof) are from different species;
(vi) MIS and IF1 protein/fragment (or sequence variant thereof) are from different species, optionally wherein the former is from a species that will be administered with the fusion protein and the latter is from a different species, preferably from a longer-living species than the subject (to be administered) species, preferably from a very long-lived species (e.g. bowhead or blue whale);
(vii) MIS and IF1 protein/fragment (or sequence variant thereof) are from same species;
(viii) IF1 protein/fragment is from Bos taurus/human/bowhead whale/blue whale/mouse/rat/naked mole rat (or sequence variant thereof), MIS is from a different species;
(ix) MIS is from human, and IF1 protein/fragment (or sequence variant thereof) is from a different species, optionally Bos taurus/whale/bowhead whale/blue whale/mouse/rat/naked mole rat;
(x) MIS is from mouse, and IF1 protein/fragment (or sequence variant thereof) is from a different species, optionally Bos taurus/whale/bowhead whale/blue whale/rat/naked mole rat;
(xi) MIS is from a species, and IF1 protein/fragment (or sequence variant thereof) is from a longer-living species (higher maximal lifespan);
(xii) MIS is from a species, and IF1 protein fragment (or sequence variant thereof) is from a shorter-living species (lower maximal lifespan);
(xiii) MIS is from a species, and IF1 protein fragment (or sequence variant thereof) has more residues from the N-terminal than C-terminal half of IF1 protein, preferably from a longer-living species (higher maximal lifespan);
(xiv) MIS is from a species, and IF1 protein fragment (or sequence variant thereof) has more residues from the C-terminal than N-terminal half of IF1 protein, preferably from a shorter-living species (lower maximal lifespan);
(xv) MIS is from a species, and the IF1 protein fragment (or sequence variant thereof) is from a different species, preferably a longer-living species (higher maximal lifespan), and comprises (or consists of) {using “mature” [without MIS] IF1 protein numbering} one or more of (or sequence variant thereof) 10-47, 13-47, 14-47, 1-56, 1-57, 1-58, 1-59, 1-60, 10-56, 10-57, 10-58, 10-59, 10-60, 42-58, 42-59 residues;
(xvi) MIS is from a species, and the IF1 protein fragment (or sequence variant thereof) is from a different species, preferably a shorter-living species (lower maximal lifespan), wherein it is shorter than 40 (or 35, or 30, or 25, or 20, or 15, or 10) amino acids, and contains the H49 residue (using “mature” [without MIS] IF1 protein numbering);
(xvii) is (in total or in part[s]) retroinverse, optionally wherein the Mitochondrial Import Sequence (MIS) is not retroinverse;
(xviii) MIS is excluded from being retroinverse, but other part(s) can be;
(xix) MIS is not retroinverse but the IF1 protein/fragment (or sequence variant thereof) is retroinverse (in entirety or in part[s]);
(xx) MIS is not retroinverse but the IF1 protein/fragment (or sequence variant thereof) and/or Cell Penetrating Peptide (CPP) sequence is retroinverse (in entirety or in part[s]);
(xxi) CPP component(s) is one or more of a Tat sequence(s) (and/or sequence variant(s) thereof of the art), Penetratin sequence(s) (and/or sequence variant(s) thereof of the art), poly-Arginine sequence(s) (and/or sequence variant(s) thereof of the art), optionally YGRKKRRQRRRG [SEQ ID NO:446] (optionally wherein the terminal glycine is absent), optionally RRRRRRRG [SEQ ID NO:461] (optionally wherein the terminal glycine is absent), optionally wherein one or more of the amino acids can be corresponding D-amino acids, optionally wherein part(s) or all of the CPP component(s) is retroinverse;
(xxii) epitope/affinity tag component(s) comprises (or consists of) one or more of poly-histidine, any of SEQ ID NO:130 to SEQ ID NO:144 e.g. HHHHHHDYDDDDK [SEQ ID NO:136];
(xxiii) CPP component(s) is flanked on one or both sides, optionally just on its C-terminal end (which is concatenated to the MIS component), by zero or more glycine and/or proline residues, optionally between 0 and 5 residues, optionally 1 such residue;
(xxiv) CPP component(s) is bound to the remainder of the fusion protein by a disulphide (by judicious insertion and/or substitution of cysteine residues) or peptide bond(s), or mixture thereof (i.e. some of the IF1 fusion proteins have their CPP bound by a disulphide bond, others by a peptide bond), optionally wherein a cysteine residue is at the C-terminal end of the CPP, which is disulphide bonded to an inserted/substituted N-terminal/internal cysteine in the MIS or IF1 protein/fragment (or sequence variant thereof) component of the fusion protein, optionally wherein the cysteine is (using “mature” [without MIS] IF1 protein numbering) present at (in legacy to the IF1 protein/fragment, or sequence variant thereof, used e.g. if from gray whale), or substituted into, the 37^{th} position;
(xxv) modified at its N- and/or C-terminal ends, optionally amidation/esterification of the C-terminus and/or acylation (e.g. acetylation) of the N-terminus;
(xxvi) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) conjugated/acylated to its N-terminus, non-limiting e.g. myristoyl/palmitoyl/stearoyl group acylated to N-terminus;
(xxvii) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(xxviii) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated;
(xxix) part(s) or all is cyclic, optionally bicyclic, optionally where it is rendered bicyclic by judicious insertion of cysteine residues which confer attachment to a scaffold structure by thioether and/or disulphide bonds, optionally wherein, for a IF1 protein/fragment (or sequence variant thereof) containing fusion protein, if present, the CPP sequence(s) (optionally a CPP sequence(s) with precedent use in a bicyclic form from the literature) is confined to one cycle, and the MIS and IF1 protein/fragment (or sequence variant thereof) is confined to the other cycle of a bicyclic structure, optionally wherein the sequence attached to the scaffold is of the form (where IF1 below can refer to a “mature” IF1 protein(s) and/or a IF1 sequence variant(s)/fragment(s)/fragment sequence variant(s) thereof): Cys-CPP-Cys-MIS-IF1-Cys;
(xxx) N^{α}-alkylated (e.g. N^{α}-methylated) at one or more places;
(xxxi) comprises (or consists of) one or more corresponding (to its sequence) D-amino acids;
(xxxii) is (in total or in part[s]) retroinverse, optionally wherein if it contains a Mitochondrial Import Sequence (MIS), this is not retroinverse, optionally wherein the Mitochondrial Import Sequence (MIS) is not retroinverse and the IF1 protein/fragment (or sequence variant thereof) and/or Cell Penetrating Peptide (CPP) sequence is retroinverse (in entirety or in part[s]);
(xxxiii) at least one of its carboxylic groups is esterified, optionally such that one or more of its carboxyl (COOH) groups is replaced by the group (or analogue thereof):

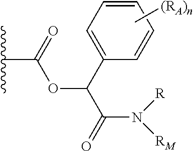

wherein RA is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example:

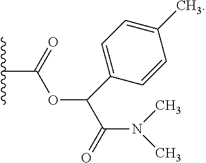 [SMILES: *C(=O)OC(C(=O)N(C)C)c1ccc(C)cc1]

A (optionally produced/isolated/purified/substantially purified/partially purified) peptide/protein comprising (or consisting of) at least one sequence selected from SEQ ID NO:166 to SEQ ID NO:438, or a fragment thereof (for non-limiting example wherein the epitope/affinity tag component [if present] is absent, and/or the Cell Penetrating Peptide component [if present] is absent), or concatenated fragments thereof, and/or a sequence variant(s) thereof (which is very preferably functional {can inhibit/reduce F₁F₀ ATP hydrolysis in a cell and/or in a Sub-Mitochondrial Particle [SMP] assay of F₁F₀ ATP hydrolysis}, optionally incorporating one or more conservative substitutions), optionally wherein one or more of the listed options in one or more of Claims 12-15 apply to the sequence[s] (non-limiting e.g. associated with a pharmaceutically/cosmetically acceptable salt[s], esterified, modified at N- and/or C-terminal ends, N-terminal pre-sequence attached, cholesterol derivative(s) and/or fatty acid(s) [or derivative(s) thereof] attached, cyclized, bicyclic, corresponding D-amino acid at one or more places, one or more parts are retroinverse, N^{α}-alkylated {e.g. N^{α}-methylated} etc. [all combinations contemplated except those that are mutually exclusive]).

A (optionally produced/isolated/purified/substantially purified/partially purified) modified (optionally conservatively), and functional (can inhibit/reduce F₁F₀ ATP hydrolysis in a cell and/or in a Sub-Mitochondrial Particle [SMP] assay of F₁F₀ ATP hydrolysis), sequence variant(s) of a protein(s)/peptide(s) of one or more of Claims 11-16.

### Polynucleotide, Vector, Cell, Gene Therapy, Transgenic Organism, Medicament Thereof

A (optionally produced/isolated/purified/substantially purified/partially purified) polynucleotide, optionally cDNA, encoding at least one peptide/protein sequence from one or more of Claims 11-17,
optionally wherein one or more of the codons, optionally all the codons, used for each amino acid are the most (or one of the most) frequently used for each amino acid in the codon bias of at least one species that will express the polynucleotide;
and/or
wherein the polynucleotide comprises (or consists of) one or more sequences selected from SEQ ID NO:1426 to SEQ ID NO:1684,
   or a pharmaceutical/cosmetic composition thereof.

A vector/plasmid of the art (non-limiting e.g. liposome, nanoparticle, lipid nanoparticle [LNP] etc.), or a pharmaceutical/cosmetic composition thereof, comprising at least one polynucleotide of Claim 18; one or more vectors/plasmids (of the art) each comprising at least one polynucleotide of Claim 18.

A cell comprising at least one vector/plasmid of Claim 19; one or more cells each comprising at least one vector/plasmid of Claim 19; optionally wherein the cell(s) can be bacterial (non-limiting e.g. E. Coli), yeast (non-limiting e.g. Saccharomyces cerevisiae), immortalized mammalian (non-limiting e.g. human) cell line, insect cell, or other cell type(s) used for recombinant protein expression in the art.

A method for producing/manufacturing a protein(s)/peptide(s) from Claims 11-17 comprising culturing (e.g. in/atop a nutrient medium) one or more cells of Claim 20 under conditions suitable for the expression of at least one polynucleotide of Claim 18, and recovering said protein(s)/peptide(s) therefrom, optionally by way of an epitope/affinity tag sequence component, optionally wherein this tag is then removed, optionally by the epitope/affinity tag sequence being connected to one end of the desired peptide/protein sequence(s), optionally the N-terminal end, by a cleavable linker sequence that is cleaved.

A gene therapy/vector of the art, or a pharmaceutical/cosmetic composition thereof, comprising at least one polynucleotide of Claim 18, optionally a gene therapy/vector of the art for (disproportional) delivery to:
(i) one or more skin/scalp cells; and/or
(ii) one or both eyes/ears; and/or
(iii) one or more brain regions and/or one or more brain cell/neuron/glia types/populations; and/or
(iv) a population(s) of cells/body region(s), e.g. a population of brain cells/brain region(s)/eye cells, that tends to age faster, and/or lose optimal function earlier in life, than other parts of the body (in that species), optionally wherein this loss is a drive to an age-correlated disease(s)/disorder(s) e.g. a neurodegenerative disease (e.g. Parkinson's disease), e.g. an age-correlated eye disorder(s)/disease(s) such as Age-related Macular Degeneration (AMD);
optionally wherein the gene therapy vector is an Adeno-Associated Virus (AAV), optionally AAV9 or AAV2;
especially preferred is the use of a gene therapy vector that is used in an FDA/EMA approved gene therapy and/or that has passed Phase I clinical trialing or/and otherwise proven safe in humans.

A transgenic organism, preferably/restrictively a non-human transgenic organism, optionally a transgenic microorganism, optionally a non-human transgenic mammal, optionally a transgenic mouse, containing at least one polynucleotide of Claim 18.

Use of at least one peptide/protein sequence of Claims 11-17, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of Claim 18 [and/or at least one vector(s) of Claim 19, and/or gene therap(y/ies) of Claim 22, and/or at least one cell(s) of Claim 20, and/or at least one transgenic organism(s) of Claim 23] in/for the manufacture of a medicament or a pharmaceutical/cosmetic composition;
use of at least one peptide/protein sequence of Claims 11-17, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of Claim 18 [and/or at least one vector(s) of Claim 19, and/or at least one gene therap(y/ies) of Claim 22, and/or at least one cell(s) of Claim 20, and/or at least one transgenic organism(s) of Claim 23] in/for the manufacture of a medicament for the treatment/amelioration/prevention/combat/reversal/slowing/delaying of aging (and/or increasing lifespan and/or healthspan), and/or an unwanted/undesirable aspect(s)/sign(s) of aging and/or an age-correlated (risk of incidence increases with subject age) disorder(s)/disease(s) (e.g. a neurodegenerative disease[s]), in a subject;
use of at least one peptide/protein sequence of Claims 11-17, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of Claim 18 [and/or at least one vector(s) of Claim 19, and/or at least one gene therap(y/ies) of Claim 22, and/or at least one cell(s) of Claim 20, and/or at least one transgenic organism(s) of Claim 23] in/for the manufacture of a medicament/cosmetic/supplement for the treatment/amelioration/prevention/combat/reversal/slowing/delaying of skin/scalp aging and/or one or more signs of skin/scalp aging/photoaging/age-correlated damage (non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.), optionally in a pharmaceutical/dermopharmaceutical/cosmetic/supplement composition/form of the art for topical/skin/scalp/transdermal (e.g. human skin/scalp) administration, optionally a cream/lotion/spray/gel/oil/liquid/foam/paste/aerosol/butter/patch/make-up/shampoo/soap thereof.

### Novel IF1 Proteins

A method of fusing fragment(s)/residue(s) of two or more different (from different species) IF1 proteins to generate a novel IF1 protein/fragment sequence, which is functional (it can inhibit/reduce F₁F₀ ATP hydrolysis e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably with greater inhibitory activity at pH 8 than any of the contributing IF1 proteins, optionally wherein fragment(s)/residue(s) of the IF1 protein sequence of a longer living species (e.g. longer maximal lifespan) is substituted into the equivalent position(s) of the IF1 protein/fragment sequence of a shorter living species in order to generate a novel IF1 protein/fragment sequence, optionally wherein a consensus sequence (most commonly observed residue is incorporated at each position) is generated from IF1 protein sequences from a number of different long-lived species, to generate a novel IF1 protein/fragment sequence.

### Screening Method

Method of screening for least one IF1 protein fragment that can inhibit/reduce F₁F₀ ATP hydrolysis in a Sub-Mitochondrial Particle [SMP] assay of F₁F₀ ATP hydrolysis, at alkaline pH (e.g. pH 8), in which endogenous/native IF1 protein is not removed,
preferably wherein a number of different IF1 protein fragments are systematically tested, preferably by one or more of the following methods:
   (1) wherein the first IF1 protein fragment tested consists of the most C-terminal (last) residue of an IF1 protein (non-limiting e.g. of Bos taurus), the second fragment tested consists of the last two residues, the third fragment consists of the last three residues, the fourth fragment consists of the last four residues, and this testing is iterated in this fashion, adding a residue each time (optionally testing until the N-terminal end of the IF1 protein is reached, or stopping before this, optionally stopping once the 47^{th} or 42^{nd} residue [from the N-terminal end, using “mature” {without MIS} IF1 protein numbering] is reached, or when a residue nearby is reached); then, with each fragment found to have inhibition/reduction of F₁F₀ ATP hydrolysis activity, the fragment sequence is tested again but with its most C-terminal (last) absent, and then again iteratively, each time with one more amino acid removed from its C-terminal end, until the activity is lost or until there are no residues remaining;
   (2) wherein the first IF1 protein fragment tested consists of IF1 protein residues (using “mature” [without MIS] IF1 protein numbering): 42-58, and then in the next test, the 43-58 fragment is tested, then 44-58, then 45-58 etc., wherein with each new test, the fragment has one less amino acid at its N-terminal end, until there are no more fragments left to test (the same method but coming instead from the C-terminal end is contemplated also);
optionally wherein one or more of the following apply (wherein all possible combinations are contemplated {including all possible combinations of elements/descriptors within, and across, different bullet points} except those that are mutually exclusive):
   (i) the SMPs are from a mammal;
   (ii) the SMPs are from a species that is to be administered with the IF1 protein fragment(s) (and/or sequence variant(s) thereof), or fusion protein(s) thereof, selected by the assay, and/or from a closely/not too distantly related species (e.g. if humans are to be administered, the SMPs can be from bovine);
   (iii) the assayed IF1 protein fragment(s) (and/or sequence variant(s) thereof) is/are from a mammal;
   (iv) the IF1 protein fragment(s) (and/or sequence variant(s) thereof) assayed is/are from a species that is to be administered with the IF1 fragment(s), or fusion protein(s) thereof, selected by the assay, and/or from a closely/not too distantly related species;
   (v) the IF1 protein fragment(s) (and/or sequence variant(s) thereof) is from a short(er)-living species, but preferably a species not too evolutionarily divergent from a species that is to be administered with the IF1 fragment(s) (and/or sequence variant(s) thereof), or fusion protein(s) thereof, selected by the assay, wherein shorter living mammals have more strongly/tightly joined IF1 tetramers (and higher oligomers), wherein they are bound to each other in their C-terminal halves, and so a C-terminal IF1 fragment from a shorter-living mammal binds a C-terminal part of an IF1 protein more tightly (if the evolutionary distance between them isn't too great);
optionally wherein the method(s) is repeated with fragments from the IF1 protein of a different species, optionally wherein it is performed with fragments from the IF1 protein of a number of different species;
in an optional next step(s), for each of the selected peptides, or for only one or more of the most potent (e.g. low EC₅₀ against F₁F₀ ATP hydrolysis), an alanine scan is performed, wherein each residue position is iteratively substituted to alanine, and the ability to inhibit/reduce F₁F₀ ATP hydrolysis at alkaline pH (e.g. pH 8), in an SMP assay of F₁F₀ ATP hydrolysis (with endogenous IF1 protein present and not removed), is assayed each time, to identify key amino acid residues, and, by contrast, those that can be changed without (much) loss of activity (or that actually increase activity), wherein sequence variants with an amino acid changed at one or more of these positions are componentry to this disclosure.
also componentry are sequence variants wherein an amino acid(s) is changed to be a different amino acid that a different species has at the equivalent position in their IF1 protein;
functional sequence variants of all these fragments are contemplated;
optionally wherein each IF1 protein fragment(s) (or sequence variant(s) thereof) selected by this method (i.e. shown to reduce F₁F₀ ATP hydrolysis) is tested in an SMP assay of F₁F₀ ATP synthesis, optionally wherein if it appreciably reduces F₁F₀ ATP synthesis also, it is discounted.

A Peptide Identifiable by a Method of Claim 26.

A method of Claim 26 wherein it is not an IF1 protein fragment(s) that is assayed but instead a fragment(s) (e.g. less than one or more of 60, 50, 40, 20, 10, 5 amino acids long) of a protein(s) found in ATP synthase (and/or sequence variant(s) thereof), or a concatenation(s) of fragments from same/different proteins found in ATP synthase (and/or sequence variant(s) thereof), optionally from β sub-unit of F₁, optionally wherein an ATP synthase sequence(s) that some or all of the C-terminal part of IF1 protein binds (when IF1 protein inhibits F₁F₀ ATP hydrolysis) is prioritized for testing (e.g. sequence(s) that some or all of the IF1 protein C-terminal half binds (e.g. that part after its 47h residue [using “mature” {without MIS} IF1 protein residue numbering])),
optionally wherein one or more of the following apply (wherein all possible combinations are contemplated {including all possible combinations of elements/descriptors within, and across, different bullet points} except those that are mutually exclusive):
   (i) the ATP synthase sequence(s) is from a mammal;
   (ii) the ATP synthase sequence(s) is from a species that is to be administered with the ATP synthase fragment(s), or fusion protein(s) thereof, selected by the assay, and/or from a closely/not too distantly related species;
optionally wherein each ATP synthase protein fragment(s) (or sequence variant(s) thereof) selected by this method (i.e. shown to reduce F₁F₀ ATP hydrolysis) is tested in an SMP assay of F₁F₀ ATP synthesis, optionally wherein if it appreciably reduces F₁F₀ ATP synthesis also, it is discounted.

### Fusion Proteins Thereof

A fusion protein (and/or a pharmaceutically/cosmetically acceptable salt, solvate, hydrate, prodrug, liposome, nanoparticle [e.g. lipid nanoparticle, LNP] or other vector of the art thereof) comprising
either a
(i) N-terminal Cell Penetrating Peptide (CPP) sequence(s) concatenated to a Mitochondrial Import Sequence(s) (MIS(s), preferably for an IF1 protein), or
(ii) N-terminal Mitochondrial Import Sequence(s) (MIS(s), preferably for an IF1 protein), concatenated to either a
   (a) (preferably “mature” i.e. without its MIS) novel IF1 protein(s)/fragment(s) selected by a method of Claim 25, or
   (b) IF1 protein fragment(s) (and/or sequence variant(s) thereof) selected by a method of Claim 26, or
   (c) ATP synthase fragment(s) (and/or sequence variant(s) thereof) selected by a method of Claim 28.
      Gene Therapy with ANY IF1 Protein/Fragment (or Sequence Variant Thereof)

A vector of the art for a polynucleotide, preferably a gene therapy vector of the art, optionally an Adeno-Associated Virus (AAV) [optionally AAV2 administered to the eye(s)], comprising at least one polynucleotide coding for at least one [any] IF1 protein/fragment
(or sequence variant thereof,
optionally wherein {using “mature” [without MIS] IF1 protein numbering} it has a H49K (or H49A or H49R) substitution and, if its 14^{th} residue is not alanine, it is substituted to be alanine),
optionally derived from that of a long lived mammal species (e.g. whale species [longer living species preferred e.g. bowhead or blue whale]) and/or long-lived reptile species e.g. a tortoise/turtle/terrapin etc.)
or a pharmaceutical/cosmetic composition thereof;
especially preferred is the use of a gene therapy vector that is used in an FDA/EMA approved gene therapy and/or that has passed Phase I clinical trialing or/and otherwise proven safe in humans.
   Medicament/Cosmetic/Supplement with ANY IF1 Protein/Fragment (or Sequence Variant Thereof)

Use of at least one (optionally produced/isolated/purified/substantially purified/partially purified) [any] IF1 protein/fragment (or sequence variant thereof), and/or a fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or a fusion protein(s) thereof (optionally containing a CPP sequence(s)) [and/or vector(s)/plasmid(s)/liposome/(s)/nanoparticle(s)/gene therap[y/ies] thereof, and/or cell(s)/transgenic cell(s)/transgenic organism(s) thereof], in/for the manufacture of a medicament or a pharmaceutical/cosmetic composition;
use of at least one (optionally produced/isolated/purified/substantially purified/partially purified) [any] IF1 protein/fragment (or sequence variant thereof), and/or fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or fusion protein(s) thereof (optionally containing a CPP sequence(s)) [and/or vector(s)/plasmid(s)/liposome(s)/nanoparticle(s)/gene therap[y/ies] thereof, and/or cell(s)/transgenic cell(s)/transgenic organism(s) thereof], in/for the manufacture of a medicament for the treatment/amelioration/prevention/combat/reversal/slowing/delaying of aging (and/or increasing lifespan and/or healthspan), and/or an unwanted/undesirable aspect(s)/sign(s) of aging and/or an age-correlated (risk of incidence increases with subject age) disorder(s)/disease(s) (e.g. a neurodegenerative disease[s]), in a subject;
use of at least one (optionally produced/isolated/purified/substantially purified/partially purified) [any] IF1 protein/fragment (or sequence variant thereof), and/or a fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or a fusion protein(s) thereof (optionally containing a CPP sequence(s)) [and/or vector(s)/plasmid(s)/liposome(s)/nanoparticle(s)/gene therap[y/ies] thereof, and/or cell(s)/transgenic cell(s)/transgenic organism(s) thereof], in/for the manufacture of a medicament/cosmetic/supplement for the treatment/amelioration/prevention/combat/reversal/slowing/delaying of skin/scalp aging and/or one or more signs of skin/scalp aging/photoaging/age-correlated damage (non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.), optionally in a pharmaceutical/cosmetic/supplement composition of the art for topical/skin/scalp/transdermal (e.g. human skin/scalp) administration, optionally a cream/lotion/spray/gel/oil/liquid/foam/paste/aerosol/butter/patch/make-up/shampoo/soap thereof,
   optionally wherein one or more of the following apply to it (wherein all possible combinations are contemplated {including all possible combinations of elements/descriptors within, and across, different bullet points} except those that are mutually exclusive):
   (i) comprises at least one [any] IF1 protein (or sequence variant thereof) and at least one [any] IF1 protein fragment (or sequence variant thereof);
   (ii) comprises multiple different IF1 protein and/or IF1 protein fragment sequences, optionally wherein one or more are sequence variants, optionally wherein two or more are different (optionally overlapping) fragments from the same IF1 protein (from the same species) or from the same sequence variant thereof;
   (iii) at least one IF1 protein(s)/fragment(s) (or sequence variant thereof) is from a long-lived species (e.g. with a long maximal lifespan) e.g. from a long lived mammal species (e.g. whale species [longer living species preferred e.g. bowhead or blue whale]) and/or long-lived reptile species e.g. a tortoise/turtle/terrapin etc.;
   (iv) at least one IF1 protein(s)/fragment(s) (or sequence variant thereof) is from human.

A process for making a pharmaceutical/cosmetic/supplement composition/medicament of Claim 31 comprising combining an amount (preferably an effective amount e.g. a therapeutically/cosmetically effective amount) of at least one (optionally produced/isolated/purified/substantially purified/partially purified) [any] IF1 protein/fragment (or sequence variant thereof), and/or a fusion protein(s) thereof (optionally containing a CPP sequence(s)), optionally at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or a fusion protein(s) thereof (optionally containing a CPP sequence(s)) [and/or vector(s)/plasmid(s)/liposome(s)/nanoparticle(s)/gene therap[y/ies] thereof, and/or cell(s)/transgenic cell(s)/transgenic organism(s) thereof], with a pharmaceutically/cosmetically acceptable vehicle(s).

### New Cosmetic/Therapeutic Methods

A method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises reducing F₁F₀ ATP hydrolysis in the subject.

A method of Claim 33 wherein F₁F₀ ATP hydrolysis is reduced in the subject by inhibiting F₁F₀ ATP hydrolysis in the subject.

A method of Claim 33 wherein F₁F₀ ATP hydrolysis is reduced in the subject by administering at least one F₁F₀ ATP hydrolysis inhibitor to the subject.

A method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises increasing the amount of (at least one type of) IF1 protein (and/or sequence variant(s) thereof) in the subject.

A method of Claim 36, wherein at least some of the extra IF1 protein (and/or sequence variant thereof) has a Mitochondrial Import Sequence (MIS) attached (preferably by peptide bond to its N-terminal end) and/or at least some of the extra IF1 protein (and/or sequence variant thereof) doesn't have a Mitochondrial Import Sequence (MIS) attached.

A method of Claim 36, wherein at least some of the extra IF1 protein (and/or sequence variant thereof) has a Mitochondrial Import Sequence (MIS) attached (preferably by peptide bond to its N-terminal end) that is the same as an MIS that the subject's/subject's species' uses to transport one or more of its endogenous/native proteins from the cytoplasm to the mitochondrial matrix, optionally the MIS that the subject's/subject's species' uses for its endogenous/native IF1 protein.

A method of Claim 36 wherein at least some of the extra IF1 protein has the same sequence as the subject's/subject's species' endogenous/native IF1 protein.

A method of Claim 36 wherein at least some of the extra IF1 protein is an IF1 protein sequence from a different species, optionally a longer-living (higher maximal lifespan) species than the subject's species, optionally a very long-lived species (high maximal lifespan).

A method of Claim 36, wherein at least some of the extra IF1 protein is an IF1 protein (from same or different species than the subject, optionally from a longer-living species, optionally from a very long-lived species) sequence variant with (using mature {without MIS} IF1 protein residue numbering) lysine or alanine or arginine as its 49^{th} residue, and/or alanine as its 14^{th} residue.

A method of Claim 36, wherein the increase in the amount of (at least one type of) IF1 protein (and/or sequence variant thereof) is because of at least one IF1 protein (and/or sequence variant thereof) coding polynucleotide sequence (e.g. an ATPIF1 gene or sequence variant thereof), and/or vector(s)/plasmid(s)/liposome(s)/nanoparticle(s)/gene therap(y/ies) thereof [and/or cell(s) thereof], or at least one pharmaceutical/cosmetic composition thereof, introduced into the subject (and/or an ancestor of the subject).

A method of Claim 36, wherein a minority of the subject's cells have an increased amount of (at least one type of) IF1 protein (and/or sequence variant(s) thereof).

A method of Claim 36, wherein a majority, optionally all, of the subject's cells have an increased amount of (at least one type of) IF1 protein (and/or sequence variant(s) thereof).

A method of Claim 36, wherein the subject has an increased amount of (at least one type of) IF1 protein (and/or sequence variant(s) thereof) in one or more of their body parts/organs/tissues/cell populations/brain regions (and/or one or more sub-parts/regions thereof).

A method of Claim 36, wherein the subject has an increased amount of (at least one type of) IF1 protein (and/or sequence variant(s) thereof) in two or more of their body parts/organs/tissues/cell populations/brain regions (and/or one or more sub-parts/regions thereof).

A method of Claim 36, wherein the subject has an increased amount of (at least one type of) IF1 protein (and/or sequence variant(s) thereof) in one or more cells of one or more body parts/organs/tissues/cell populations/brain regions (and/or one or more sub-parts/regions thereof) that tend to age fastest and/or where slower aging is most desired by (or most recommended to, optionally by a healthcare worker) the subject and/or the aging of which is particularly/possibly associated with/can drive an age-correlated (risk/incidence increases with age) disorder(s)/disease(s) and/or the failing of which can be a cause of death and/or the failing of which can be a common cause of death and/or the failing of which can be an unwanted cause of death.

A method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises administering to the subject (and/or the subject self-administering), systemically and/or locally/topically to the subject's body part(s)/organ(s)/tissue(s)/cell population(s)/cell(s) where the effect(s) is (most) sought (e.g. to one or more areas of skin/scalp e.g. one or more areas of the face, e.g. to one or both eyes/ears, e.g. to one or more joints), optionally as part of a pharmaceutical/cosmetic/supplement composition/medicament, an amount (preferably an effective amount e.g. a therapeutically/cosmetically effective amount) of at least one compound/composition that has the ability/property (for non-limiting example in a Sub-Mitochondrial Particle [SMP] assay of F₁F₀ ATP hydrolysis) to preferentially/disproportionally inhibit/reduce the activity of the ATP-hydrolysing, as compared to the ATP-synthesizing, mode of ATP synthase;
preferably wherein there is a notable/sizeable differential between the compound's EC₅₀ F₁F₀ ATP synthesis and a smaller valued EC₅₀ F₁F₀ ATP hydrolysis (for non-limiting example in a Sub-Mitochondrial Particle [SMP] assay of F₁F₀ ATP hydrolysis), wherein greater disparity is more preferred, for example (in order of increasing preference) one or more of >10, >100, >1000, >5000 times difference, optimally wherein the compound inhibits/reduces the activity of the ATP-hydrolysing mode of ATP synthase and does not, or does not appear to, or only minimally, or much less, inhibits/reduces the activity of the ATP-synthesizing mode of ATP synthase.

A method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises administering to the subject (and/or the subject self-administering), systemically and/or locally/topically to the subject's body part(s)/organ(s)/tissue(s)/cell population(s)/cell(s) where the effect(s) is (most) sought (e.g. to one or more areas of skin/scalp e.g. one or more areas of the face, e.g. to one or both eyes/ears, e.g. to one or more joints), optionally as part of a pharmaceutical/cosmetic/supplement composition/medicament, an amount (preferably an effective amount e.g. a therapeutically/cosmetically effective amount) of at least one proteinaceous compound/peptide/protein/polypeptide/amino-acid sequence based compound, wherein this compound(s)/composition(s) has the ability/property (for non-limiting example in a Sub-Mitochondrial Particle [SMP] assay of F₁F₀ ATP hydrolysis) to preferentially/disproportionally inhibit/reduce the activity of the ATP-hydrolysing, as compared to the ATP-synthesizing, mode of ATP synthase; preferably wherein there is a notable/sizeable differential between the compound's EC₅₀ F₁F₀ ATP synthesis and a smaller valued EC₅₀ F₁F₀ ATP hydrolysis (for non-limiting example in a Sub-Mitochondrial Particle [SMP] assay of F₁F₀ ATP hydrolysis), wherein greater disparity is more preferred, for example (in order of increasing preference) one or more of >10, >100, >1000, >5000 times difference, optimally wherein the compound inhibits/reduces the activity of the ATP-hydrolysing mode of ATP synthase and does not, or does not appear to, or only minimally, or much less, inhibits/reduces the activity of the ATP-synthesizing mode of ATP synthase;
and/or the (self-) administration to the subject of at least one polynucleotide sequence that codes for an aforementioned protein(s)/peptide(s) of this present claim, which inhibits/reduces F₁F₀ ATP hydrolysis, preferably with characteristics as aforementioned, and/or at least one vector(s)/plasmid(s)/liposome(s)/nanoparticle(s) thereof, and/or at least one cell(s) thereof, and/or at least one gene therapy thereof, and/or at least one transgenic organism/virus thereof, and/or at least one pharmaceutical/cosmetic composition thereof.

A method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises administering to the subject (and/or the subject self-administering), systemically and/or locally/topically to the subject's body part(s)/organ(s)/tissue(s)/cell population(s)/cell(s) where the effect(s) is (most) sought (e.g. to one or more areas of skin/scalp e.g. one or more areas of the face, e.g. to one or both eyes/ears, e.g. to one or more joints), optionally as part of a pharmaceutical/cosmetic/supplement composition/medicament, an amount (preferably an effective amount e.g. a therapeutically/cosmetically effective amount) of (optionally produced/isolated/purified/substantially purified/partially purified) at least one [any] IF1 protein/fragment (or sequence variant thereof) and/or (optionally produced/isolated/purified/substantially purified/partially purified) at least one fusion protein comprising at least one [any] IF1 protein/fragment (or sequence variant thereof), and/or at least one pharmaceutically/cosmetically acceptable salt, solvate, hydrate, prodrug, liposome, nanoparticle (e.g. lipid nanoparticle, LNP) or other vector of the art thereof, optionally comprising an N-terminal Cell Penetrating Peptide (CPP) sequence(s) concatenated to a Mitochondrial Import Sequence(s) (MIS(s)) concatenated to a (preferably “mature” i.e. without its MIS) IF1 protein/fragment (or sequence variant thereof), and/or (optionally produced/isolated/purified/substantially purified/partially purified) at least one polynucleotide coding for at least one [any] IF1 protein/fragment (or sequence variant thereof), and/or a fusion protein(s) thereof, and/or a vector(s)/plasmid(s)/liposome(s)/nanoparticle(s)/gene therap[y/ies]/cell(s) thereof, and/or at least one pharmaceutical/cosmetic composition thereof.

A method of Claim 50 wherein one or more of the following applies/is true (wherein all possible combinations are contemplated {including all possible combinations of elements/descriptors within, and across, different bullet points} except those that are mutually exclusive):
(i) at least one IF1 protein/fragment (or sequence variant thereof), and/or at least one IF1 protein/fragment (or sequence variant thereof) containing fusion protein, is according to one or more of Claims 11-17, 25-29;
(ii) at least one polynucleotide is according to Claim 18;
(iii) at least one vector/plasmid is according to Claim 19;
(iv) at least one cell is according to Claim 20;
(v) at least one gene therapy is according to one or more of Claims 22, 30;
(vi) at least one medicament or pharmaceutical/cosmetic composition is according to one or more of Claims 24, 31;
(vii) at least one IF1 protein/fragment (or sequence variant thereof), and/or IF1 protein/fragment (or sequence variant thereof) component to a fusion protein, is part, or the entirety, of the subject's species' native IF1 protein, or a sequence variant thereof;
(viii) at least one IF1 protein/fragment (or sequence variant thereof), and/or IF1 protein/fragment (or sequence variant thereof) component to a fusion protein, is from a species that tends to live longer (e.g. has a greater maximal lifespan) than the subject's species, optionally/preferably from one of the most long-lived species on Earth e.g. bowhead whale;
(ix) at least one IF1 protein/fragment (or sequence variant thereof), and/or IF1 protein/fragment (or sequence variant thereof) component to a fusion protein, is a fusion of fragment(s)/residue(s) of IF1 protein sequences from two or more different species, optionally with one or more (preferably single residue) substitutions/insertions/deletions atop at one or more positions, preferably at less than 10 positions;
(x) at least one IF1 protein/fragment (or sequence variant thereof), and/or IF1 protein/fragment (or sequence variant thereof) component to a fusion protein, has a Mitochondrial Import Sequence [MIS] attached (preferably by peptide bond to its N-terminal end) that is the same as an MIS that the subject's/subject's species' uses to transport one or more of its endogenous/native proteins from the cytoplasm to the mitochondrial matrix, optionally the MIS that the subject's/subject's species' uses for its endogenous/native IF1 protein;
(xi) at least one IF1 protein fragment (or sequence variant thereof) is administered;
(xii) at least one IF1 protein (or sequence variant thereof) and at least one IF1 protein fragment (or sequence variant thereof) is administered;
(xiii) multiple different IF1 protein and/or IF1 protein fragment sequences, optionally wherein one or more are sequence variants, optionally wherein two or more different (optionally overlapping) fragments from the same IF1 protein (from the same species) or from the same sequence variant thereof, are administered;
(xiv) at least one IF1 protein/fragment (or sequence variant thereof) is functional as a stand-alone/separate peptide/protein i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it confers greater inhibition of F₁F₀ ATP hydrolysis than a naturally occurring IF1 protein at pH 8;
(xv) at least one IF1 protein/fragment (or sequence variant thereof) has one or more conservative substitutions, and/or one or more non-conservative substitutions, and is functional i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater inhibitory activity against F₁F₀ ATP hydrolysis than a naturally occurring IF1 protein at pH 8;
(xvi) at least one IF1 protein/fragment (or sequence variant thereof) has notable/high sequence identity (e.g. one or more of ≥28%, ≥30%, >40%, ≥50%, ≥60%, ≥70%, ≥75%, ≥80%, ≥85%, >90%, ≥95%, ≥96%, >97%, ≥98%, ≥99% sequence identity and/or less than 12, or less than 10, or less than 8, or less than 6, or less than 5, or less than 4, or less than 3, or 1 to 2 single residue substitutions/insertions/deletions away from a native IF1 protein sequence or fragment(s) thereof) to the entirety, or part, of at least one IF1 protein from a species, and is functional i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater inhibitory activity against F₁F₀ ATP hydrolysis than a naturally occurring IF1 protein at pH 8.

### Accounting for Body Temperature Effect

A method of one or more of Claims 33-51 wherein administration is local/topical, instead of systemic, and so any ensuing reduction in endogenic/metabolic heat production in (and optionally around) the administered area (caused by less F₁F₀ ATP hydrolysis in that area) is compensated for by heat transfer from other body regions, especially via blood flow, maintaining (at or close to) the optimal body temperature (e.g. ˜37° C. in a mammal) in/around the administered area;
optionally wherein administration is local/topical to the skin/scalp, optionally in a pharmaceutical/cosmetic/supplement composition of the art for topical/skin/scalp/transdermal (e.g. human skin/scalp) administration, optionally a cream/lotion/spray/gel/oil/liquid/foam/paste/aerosol/butter/patch/make-up/shampoo/soap thereof, optionally wherein this administration acts to slow/delay/reverse/treat/ameliorate/prevent/combat skin/scalp aging (e.g. one or more signs of skin aging/photaging/age-correlated damage: non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.);
optionally wherein administration is local/topical to one or both eyes, optionally in a pharmaceutical composition of the art for eye administration, optionally one or more of eye drop(s), intravitreal injection(s), contact lens coating/solution (optionally wherein the contact lens has little to no refractive ability or wherein the contact lens is prescriptive to the refractive defect/error of the subject's eye(s)) thereof, optionally wherein this administration acts to slow/delay/reverse/treat/ameliorate/prevent/combat eye(s) aging and/or at least one eye(s) aging related disease/disorder, including any eye disease(s)/disorder(s) whose likelihood of onset increases with age and/or worsens with age, including (to illustrate and not restrict) age-related macular degeneration (AMD, early/intermediate/late), age-related wet macular degeneration, neovascular/wet AMD, dry AMD, Geographic atrophy (GA), wet and dry AMD in the same eye(s), Stargardt's macular degeneration, Best vitelliform macular dystrophy, diabetic retinopathy, proliferative diabetic retinopathy, diabetic macular edema, vision loss, progressive vision impairment, myopia (short-sightedness), degenerative myopia, hyperopia (far-sightedness), accommodative dysfunction, glaucoma, progressive glaucoma, cataract formation, retinal degeneration, progressive retinal degeneration, retinitis pigmentosa, leber hereditary optic neuropathy, Fuchs spot, Best's disease, Sorsby's fundus dystrophy.

A method of one or more of Claims 33-51 wherein administration is systemic, and optionally the subject is monitored, for example by a healthcare professional(s) and/or machine substitute(s), for sign(s) of reduction in body temperature and/or the subject is located at an ambient temperature that maintains their body temperature within safe limits whilst they have an effective amount of administered compound(s)/composition(s) in their system and/or the subject wears (and/or is covered by) insulating material(s), e.g. clothing/clothes (and/or bedding/blanket(s)), and/or is in a heated/insulated space and/or hot climate, optionally exceeding 25° C. or 28° C. or 30° C. or 35° C. or 36° C. or 37° C., optionally at or around 37° C., wherein a high (e.g. in the thirties ° C. i.e. 3×° C. where x is a number between 0 and 9), but safe, ambient temperature (and/or greater bodily insulation, for example by clothing/clothes and/or bedding/blanket(s)) can permit a greater compound(s)/composition(s) dose(s) to be safely administered, wherein a preferred ambient temperature is the thermoneutral temperature for the subject with the amount of bodily insulation they have, e.g. the amount of clothing they are wearing, if any, and the amount of the administered compound(s)/composition(s) in their body/system;
optionally wherein the subject wears one or more items of clothing, and/or is sheltered, and/or is in a heated and/or insulated confinement/room/space, some or all of the time whilst they have an amount (e.g. effective amount e.g. a therapeutically/cosmetically effective amount) of the administered compound(s)/composition(s) in their body/system;
optionally wherein the subject is administered (and/or self-administers) the compound(s)/composition(s) shortly before they sleep, preferably wherein they are sheltered (e.g. inside instead of outside) and/or insulated (e.g. by bedding(s)/blanket(s), and/or clothing, and the like) whilst they sleep, optionally in a heated room/building/confinement that is set to a higher (safe) temperature than outside it.

### Structure to Function

An IF1 protein/fragment (or sequence variant thereof), or fusion protein thereof, optionally a pharmaceutically/cosmetically acceptable salt, solvate, hydrate, prodrug, liposome, nanoparticle (e.g. lipid nanoparticle, LNP) or other vector of the art thereof, wherein one or more of the following applies to it (wherein all possible combinations are contemplated {including all possible combinations of elements/descriptors within, and across, different bullet points} except those that are mutually exclusive), wherein some functional significance is disclosed in this present claim:
(i) contains one or more corresponding (to its sequence) D-amino acids;
(ii) contains one or more retroinverse regions, or all of it is retroinverse, optionally wherein the Cell Penetrating Peptide (CPP) component (if present) is retroinverse (in part or entirety) and/or the IF1 protein/fragment (or sequence variant thereof) is retroinverse (in part or entirety);
(iii) N^{α}-alkylated (e.g. N^{α}-methylated) at one or more places;
(iv) part(s) or all is cyclized, in one or more cycles;
(v) part(s) or all is bicyclic via attachment to a scaffold(s), optionally rendered bicyclic by judicious insertion of cysteine residues which confer attachment to a scaffold structure by thioether and/or disulphide bonds;
(vi) modified at its N- and/or C-terminal ends, optionally amidation/esterification of the C-terminus and/or acylation (e.g. acetylation) of the N-terminus;
   wherein one or more of the aforementioned features reduces susceptibility to protease(s) in the blood and increases peptide/protein half-life in the blood circulation of a subject (increases its plasma stability);
(vii) at least one of its carboxylic groups is esterified, optionally such that one or more of its carboxyl (COOH) groups is replaced by the group (or analogue thereof):

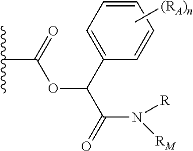

wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example:

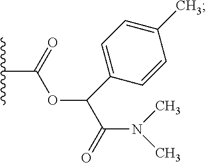 [SMILES: *C(=O)OC(C(=O)N(C)C)c1ccc(C)cc1]

wherein this esterification confers (or enhances) the ability to passage a biological/plasma membrane(s), wherein the moiety/moieties attached by an ester bond are cleaved off by esterases once the peptide/protein enters a cell;
this esterification also (sterically) reduces susceptibility to proteases in the blood and so increase plasma half-life;
(viii) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) acylated to its N-terminus, non-limiting e.g. myristoyl/palmitoyl/stearoyl group conjugated to N-terminus;
(ix) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(x) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated;
conjugated fatty acid (or derivative thereof) confers self-association and/or binding to albumin in the blood, which (sterically) decreases protease(s) access to peptide/protein, and/or slows its renal clearance, thence increasing its half-life in blood (e.g. from minutes to hours);
conjugated cholesterol/fatty acid (or derivative thereof) increases lipophibicity and confers (or enhances) the ability to passage a biological/plasma membrane(s);
when cholesterol/fatty acid (or derivative thereof) is attached by a disulphide bond, this attachment breaks once inside the reducing intracellular environment;
the pre-sequence/residue increases peptide/protein lipophibicity, which confers (or enhances) its ability to passage a biological/plasma membrane(s), and/or contributes positive charge which enhances the ability to passage into a live cell (negative inside);
in preferred cases, wherein this pre-sequence/residue/attachment is more N-terminal in the fusion protein than a Mitochondrial Import Sequence (MIS), it is inherently cleaved from the IF1 protein/fragment (or sequence variant thereof) when the MIS is cleaved off, in the mitochondrial matrix;
(xi) Cell Penetrating Peptide (CPP) component(s) to fusion protein, which comprises (or consists of) R7 [SEQ ID NO:455] or RRRRRRRG [SEQ ID NO:461] or RRRRRRRP [residues 4-11 of SEQ ID NO:453];
confers better cellular penetration than a Tat sequence; correspondent to amino acid sequences found within the human and mouse proteome and so less immunogenic in those species (amongst others) than a Tat sequence (for example); optional terminal glycine (G) or proline (P) confers flexibility at its C-terminal connection point to fusion protein;
(xii) Mitochondrial Import Sequence (MIS) and IF1 protein/fragment (or sequence variant thereof) components to fusion protein are from different species;
this permits the MIS to be from a species that will be administered the fusion protein, facilitating its better delivery into the mitochondrial matrix in that species, and the IF1 protein/fragment (or sequence variant thereof) to be from a different, longer living species (a species with a greater maximal lifespan);
(xiii) IF1 protein/fragment (or sequence variant thereof) component/entirety comprises (or consists of) that from a long-lived species, preferably at least as long-living as Bos taurus, more preferably from one of the longest-lived species/mammals on Earth e.g. bowhead or blue whale;
(xiv) one or more (or two/three/four/five/six/seven/eight/nine/ten/eleven/twelve/thirteen/fourteen/fifteen/sixteen/seventeen or more) of the following is true of the IF1 protein/fragment (or sequence variant thereof) component/entirety: (using “mature” [without Mitochondrial Import Sequence (MIS)] IF1 protein numbering): 49^{th} residue is not histidine, 14^{th} residue is not a residue that can be phosphorylated (i.e. is not serine or threonine), 26^{th} residue is not glutamic acid, 48^{th} residue is not histidine, 55^{th} residue is not histidine, 56^{th} residue is not histidine, 49^{th} residue is lysine or alanine or arginine, 14^{th} residue is alanine, 26^{th} residue is alanine or glutamine, 48^{th} residue is alanine, 55^{th} residue is alanine, 56^{th} residue is alanine, 79^{th} residue is glycine or asparagine, 76^{th} residue is lysine, 73^{rd} residue is serine, 62^{nd} residue is histidine, 82^{nd} residue is aspartic acid, 83^{rd} residue is aspartic acid, 84^{th} residue is aspartic acid, 85^{th} residue is aspartic acid, 57^{th} residue is valine, 54^{th} residue is serine or aspartic acid, 61^{st} residue is glutamine, 51^{st} residue is asparagine, 47^{th} residue is glutamic acid, 46^{th} residue is arginine, 44^{th} residue is serine, 39^{th} residue is lysine, 38^{th} residue is alanine or glutamic acid, 37^{th} residue is arginine or cysteine or lysine, 36^{th} residue is aspartic acid or glutamic acid, 29^{th} residue is histidine, 27^{th} residue is alanine, 25^{th} residue is lysine, 17^{th} residue is aspartic acid, 12^{th} residue is glycine, 11^{th} residue is serine or threonine, 10^{th} residue is serine or glycine, 9^{th} residue is glycine, 8^{th} residue is leucine or glycine, 6^{th} residue is aspartic acid or glycine, 5^{th} residue is alanine, 4^{th} residue is serine or glycine, 3^{rd} residue is glutamic acid or serine or lysine, 2^{nd} residue is glycine, 1^{st} residue is leucine;
(xv) IF1 protein/fragment (or sequence variant thereof) component/entirety is truncated, without C-terminal regions required for dimerization, teteramerization and higher oligomerization, for example it is only IF1 protein residues {using “mature” [without MIS] IF1 protein numbering}: 14-47 (or 10-47 or 13-47 or 1-56 or 1-58 or 1-60 or 10-56 or 10-58 or 10-60), preferably wherein if its 14^{th} residue isn't alanine, it is substituted to be alanine;
(xvi) IF1 protein/fragment (or sequence variant thereof) component/entirety is truncated, without the ability to inhibit F₁F₀ ATP hydrolysis itself, but wherein it binds at least one part in the C-terminal half of a complete IF1 protein, wherein (because it is truncated appropriately) its binding doesn't occlude the more N-terminal IF1 protein component that inhibits F₁F₀ ATP hydrolysis, but it does occlude one or more of the IF1 protein parts involved in tetramer (and higher) oligomerization (sequesteration) at alkaline pH (e.g. pH 8, normal pH of mitochondrial matrix), liberating IF1 dimers/monomers that can inhibit F₁F₀ ATP hydrolysis at pH 8; advantageously such an IF1 protein/fragment (or sequence variant thereof) tends to be shorter (better for intracellular delivery) than a IF1 protein/fragment (or sequence variant thereof) that can inhibit F₁F₀ ATP hydrolysis itself (e.g. aforementioned IF1 protein residues: 14-47); this particular approach relies on the endogenous IF1 protein and so works best in longer-living species which tend to have more, and/or more potent, IF1 protein; with this approach it is preferred for the IF1 protein/fragment (or sequence variant thereof) to be from the species to be administered or a shorter-living species (IF1 protein from a longer-living species binds ATP synthase more tightly/potently, and other IF1 proteins {to form IF1 protein tetramers and higher oligomers} less tightly; IF1 protein from a shorter-living species binds ATP synthase less tightly/potently and other IF1 proteins {to form IF1 protein tetramers and higher oligomers} more tightly; so shorter-living species have more tightly bound IF1 tetramers {and higher oligomers} and so a IF1 protein/fragment {or sequence variant thereof} solely designed to bind another IF1 protein is better sourced from a shorter than a longer-living species, but preferably from a species not too far evolutionarily removed from a/each species to be administered); preferably this IF1 protein/fragment (or sequence variant thereof) is less than 25 amino acids long, more preferably less than 20;
wherein one or more of the aforementioned features confers increased ability to inhibit/reduce F₁F₀ ATP hydrolysis at alkaline pH e.g. at pH 8 [which is the normal pH of the mitochondrial matrix] e.g. in a Sub-Mitochondrial Particle (SMP) assay of F₁F₀ ATP hydrolysis and/or in a cell and/or in a subject;
wherein one or more of the aforementioned features, when the peptide/protein is administered to a subject, confers increased ability to slow aging (conferring increased associated benefit(s), e.g. increased cosmetic(s) and/or therapeutic effect(s), thereof) in the subject.

### Transgenic Organisms Containing at Least One Transgenic IF1 Protein/Fragment (or Sequence Variant Thereof) Coding Nucleotide Sequence

An organism, preferably/restrictively a non-human organism, optionally a mouse, which has an H49K (or H49A or H49R), and/or S14A (or T14A), substitution in the IF1 protein produced by its mutated/modified ATPIF1 (ATP5IF1) gene,
or/and
a transgenic organism, preferably/restrictively a non-human transgenic organism, optionally a transgenic microorganism, preferably a non-human transgenic mammal, optionally a transgenic mouse, containing/expressing/constitutively expressing at least one transgenic ATPIF1 gene(s) (and/or sequence variant thereof; and/or IF1 protein/fragment [and/or sequence variant thereof] coding polynucleotide sequence[s] without (or with less) introns), optionally at least one (wherein all possible combinations are contemplated {including all possible combinations of elements/descriptors within, and across, different bullet points} except those that are mutually exclusive):
   (i) polynucleotide sequence coding for/expressing at least one IF1 protein from a longer living species (longer maximal lifespan), preferably which (using “mature” [without MIS] IF1 protein numbering) has a H49K (or H49A or H49R) substitution and, if its 14^{th} residue is not already alanine, it is substituted to be alanine; or
   (ii) polynucleotide sequence coding for/expressing an IF1 protein from blue whale (Balaenoptera musculus), preferably which (using “mature” [without MIS] IF1 protein numbering) has H49K (or H49A or H49R) and/or T14A substitutions; or
   (iii) polynucleotide sequence coding for/expressing an IF1 protein from bowhead whale (Balaena mysticetus), preferably which (using “mature” [without MIS] IF1 protein numbering) has an H49K (or H49A or H49R) substitution; or
   (iv) polynucleotide sequence coding for/expressing an IF1 protein from human, which (using “mature” [without MIS] IF1 protein numbering) has H49K (or H49A or H49R) and S14A substitutions; or
   (v) polynucleotide sequence coding for/expressing at least one IF1 protein fragment (or sequence variant thereof) concatenated at its N-terminal end to a Mitochondrial Import Sequence (MIS, for transport into the mitochondrial matrix), preferably an MIS that the organism species uses for one of one or more proteins it transports from the cytoplasm to the mitochondrial matrix, more preferably the MIS that it uses for its endogenous/native IF1 protein, wherein the IF1 protein fragment (or sequence variant thereof) can optionally be:
      (a) IF1 protein fragment (or sequence variant thereof) less than z amino acids long, wherein z is an integer selected from a group comprising 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 [different values of z are different embodiments]; or
      (b) IF1 protein fragment (or sequence variant thereof) of (using “mature” {without MIS} IF1 protein numbering) x-y, where x is an integer between 1 and 20 (or between 1 and 44, or between 1 and 84), and y is an integer between 40 and 85 (or between 40 and 85, or between 2 and 85) [different values of x and/or y are different embodiments; within their aforementioned range constraints, all possible combinations of x and y integer values are contemplated]; or
      (c) IF1 protein fragment (or sequence variant thereof) selected from a group comprising (using “mature” [without MIS] IF1 protein numbering): 1-84, 2-84, 3-84, 4-84, 5-84, 6-84, 7-84, 8-84, 9-84, 10-84, 11-84, 12-84, 13-84, 14-84, 15-84, 16-84, 17-84, 18-84, 19-84, 20-84, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 81-84, 82-84, 83-84, OR a sub-sequence/fragment of one of these aforementioned fragments; or
      (d) IF1 protein fragment (or sequence variant thereof) optionally selected from (using “mature” {without MIS} IF1 protein numbering): residues: 14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 14-60, 10-84, 14-84, 18-84, 10-50, 1-45, 42-56, 42-47, 48-56, 49-55 (or sequence variant thereof of any fragment aforementioned); or
      (e) IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60 (or sequence variant thereof); or
      (f) bowhead whale IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60; or
      (g) blue whale IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60, preferably with a T14A substitution; or
      (h) human IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60, preferably with a S14A substitution;
in one or more of:
   (i) one or both its eyes;
   (ii) forebrain/intestine/liver and at least one other brain region/body region/organ/tissue/cell population;
   (iii) forebrain/intestine/liver and at least two other brain/body regions/organs/tissues/cell populations;
   (iv) two or more of forebrain, midbrain and hindbrain;
   (v) cell type(s)/cell population(s)/tissue(s)/organ region(s)/organ(s) that tends to age faster, and underperform/fail/lose optimal function earlier (optionally wherein this underperformance/failure/optimal function loss can cause a pathology/disease in a subject [e.g. of aging] and/or sign(s) of aging/older age/old age e.g. a neurodegenerative disease), than most cell type(s)/cell population(s)/tissue(s)/organ region(s)/organ(s) in the body;
   (vi) in one or more dopamine neurons, preferably the majority/all, in the pars compacta (in the substantia nigra);
   (vii) in more than one different cell population/tissue/organ region/organ/brain region (and/or one or more sub-parts/regions thereof), optionally in at least 15% or 25% or 50% or 75% or 90% or many/majority of the organism's cells/cell populations/tissues/organs, optionally in all;
preferably wherein this modified organism has a longer healthspan and/or lifespan than is typical for its species, optionally wherein, if the modified organism is a mouse, it has a lifespan in excess of 6 and/or 5 years, optionally wherein this organism is entered into one or more lifespan and/or healthspan assays (and/or competitions e.g. M Prize or similar) of the art, optionally wherein this longer healthspan and/or lifespan is only observed when the method of Claim 56 is performed with a transgenic organism of the present claim.

A method, which is especially applicable when the organism is of a homeothermic species, of using/keeping alive an organism of Claim 55 wherein the organism lives (is reared/housed/kept) at a higher/sufficiently high safe ambient temperature (e.g. at, or at a safe temperature in excess of, 25/30/37° C.), and/or is afforded more bodily insulation, to account for it having less endogenous/metabolic heat production and a higher thermoneutral/thermo-comfortable temperature.

### Maladies of Aging

A method of any of Claims 33-53 and/or a pharmaceutical/dermopharmaceutical/cosmetic/supplement composition/medicament/peptide/protein/vector/gene therapy of any of Claims 24, 31, 54, wherein the unwanted/undesirable aspect(s)/sign(s) of aging and/or disorder(s)/disease(s) of aging (e.g. risk/incidence increases with increased age/senescence) includes (to illustrate and not restrict) geriatric aging, age-associated decline, age-related/correlated disease/disorder/condition, aging frailty, frailty, frailty syndrome, wasting, sarcopenia, muscle weakness, weakness, muscle fatigue, weight loss, cachexia, functional decline, osteoporosis, sclerosis, kyphosis, reduction in bone density, cognitive decline, neurological decline, cognitive deficit, cognitive impairment, mild cognitive impairment, depression, degenerative diseases, neurodegenerative diseases, motor-associated neurodegenerative diseases, motor neuron disease, motor neuron dysfunction, amyotrophic lateral sclerosis (ALS), primary lateral sclerosis, progressive muscular atrophy, age-related muscular atrophy, age-related fat loss, progressive bulbar palsy, progressive supranuclear palsy, pseudobulbar palsy, hereditary spastic paraplegia, Parkinson's disease, parkinsonism, Multiple System Atrophy (MSA), Progressive Supranuclear Palsy (PSP), essential tremor, resting tremor, Alzheimer's disease, Huntington's disease, spinocerebellar ataxias, Friedreich's ataxia, cerebellar ataxia, dysautonomia, dementia, frontotemporal dementia, chronic traumatic encephalopathy, memory loss, aged cognition, age/aging related cognitive decline/impairment, congential epilepsy, Batten disease, polyglutamine diseases, atherosclerosis, atherosclerotic plaque in a blood vessel, arteriosclerosis, vascular stiffening, arterial stiffness, stiffened arteries, hypertension, cardiovascular disease(s), myocardial infarction, acute myocardial infarction, angina, arrhythmia, cardiomyopathy, congestive heart failure, coronary artery disease, carotid artery disease, endocarditis, coronary thrombosis, myocardial infarction, ischemia reperfusion injury, anemia, hypertension, aortic aneurysm, cardiac diastolic dysfunction, irregularity in heart rhythm, decrease in cardiac stress tolerance, increase in the cross-sectional area of cardiomyocyte(s), hypercholesterolemia, hyperlipidemia, mitral valve prolapsed, peripheral vascular disease, cardiac stress resistance, brain aneurysm, inflammatory or autoimmune disease, cerebrovascular disease, stroke, heart failure, heart failure with preserved ejection fraction, fibrosis, idiopathic pulmonary fibrosis (IPF), pulmonary fibrosis, fibrotic disease, cardiac fibrosis, liver fibrosis, pancreatic fibrosis, oral submucosa fibrosis, cystic fibrosis, gum recession, gingival recession, oral mucositis, pulmonary disease, age-related loss of pulmonary function, chronic obstructive pulmonary disease, emphysema, bronchiectasis, coronary artery disease, hypercholesterolemia, liver disease, fatty liver disease, lysosomal storage disease, amyloidosis, systemic sclerosis, kidney disease, chronic kidney disease, renal disease, renal failure, end-stage renal disease (ESRD), renal insufficiency, glomerulosclerosis, cirrhosis, hepatic cirrhosis, hepatic insufficiency, immunosenscence, clonal hematopoiesis, Chronic Obstructive Pulmonary Disease (COPD), emphysema, breathlessness, asthma, hypertension, hypercholesterolemia, age-related thymic atrophy, chronic inflammatory disease(s), joint pain, arthritis, osteoarthritis, osteoarthritis of knee(s), arthritis (Osteo- and Rheumatoid), Juvenile Rheumatoid Arthritis (JRA), arthrosis, herniated intervertebral disc, kyphosis, degenerative disc disease, vertebral disc degeneration, tendinopathy, androgenetic alopecia, male-pattern baldness, hair loss, Idiopathic Pulmonary Fibrosis, systemic sclerosis, Psoriasis, age-related loss of cardiac/pulmonary/cognitive/vision function, decrease in cardiac stress tolerance, insulin sensitivity, poor glycemic control, diabetes, type 1 diabetes, type 2 diabetes, diabetic ulcer, diabetic retinopathy, diabetic neuropathy, diabetic nephropathy (diabetic kidney disease), diabetic ulcer, boutonneuse fever, obesity, metabolic disease/syndrome/dysfunction, inflammatory bowel disease, andropause, glaucoma, progressive glaucoma, retinal degeneration, sarcopenia, cachexia, age-related cachexia and/or sarcopenia, macular degeneration, Age-related Macular Degeneration (AMD, early/intermediate/late), age-related wet macular degeneration, neovascular/wet AMD, dry age-related macular degeneration, dry AMD, Geographic atrophy (GA), dry age-related macular degeneration with geographic atrophy, wet and dry AMD in the same eye(s), Stargardt's macular degeneration, Best vitelliform macular dystrophy, retinopathy, diabetic retinopathy, proliferative diabetic retinopathy, diabetic macular edema, age/aging-related eye disease, ophthalmological/ophthalmic disease/disorder/condition, ocular disease, vision loss, blindness, progressive vision impairment, myopia (short-sightedness), degenerative myopia, hyperopia (far-sightedness), accommodative dysfunction, cataract formation, cataract(s), retinal degeneration, progressive retinal degeneration, presbyopia, vision loss, retinitis pigmentosa, leber hereditary optic neuropathy, Fuchs spot, Best's disease, Sorsby's fundus dystrophy, vaso-obliteration in eye(s), oxygen induced vaso-obliteration, neovascularization in eye(s), hearing loss (e.g. age-related), deafness, presbycusis, tinnitus, naive T cell shortage, movement disability, nonalcoholic fatty liver disease (NAFLD), nonalcoholic steatohepatitis (NASH), immunosenescence, immune senescence, poor immune response to a vaccine(s) (so countering this improves vaccine response=improves the protection conferred by a vaccine), respiratory/urinary tract infection (RTI/UTI) especially in older/aged/elderly subjects, loss of bladder control, lower urinary tract symptoms (LUTS), Benign Prostatic Hyperplasia (BPH), hyperplasia, polycystic kidney disease, cancer, age-related cellular hypertrophy, dermatological disease/disorder, eczema, psoriasis, hyperpigmentation, nevi, rashes, atopic dermatitis, urticaria, diseases/disorders related to photosensitivity/photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, a hallmark(s) of aging, genomic instability, telomere attrition, epigenetic alteration(s), loss of proteostasis, deregulated nutrient sensing, mitochondrial dysfunction, cellular senescence, stem cell exhaustion, altered intercellular communication, homeostatic imbalance, reduced fitness, reduced reproductive fitness, infertility, female infertility, menopause, incontinence, sleep disturbances, imbalance, fear, depression, ulcers.

A method of any of Claims 33-53 and/or a pharmaceutical/dermopharmaceutcal/cosmetic/supplement composition/medicament/peptide/protein/vector/gene therapy of any of Claims 24, 31, 54, wherein the unwanted/undesirable aspect(s)/sign(s) of aging and/or disorder(s)/disease(s) of aging includes (to illustrate and not restrict) accelerated/premature aging, any accelerated/premature aging disease, any progeroid syndrome, including (to illustrate and not restrict) premature aging because of chemo-/radio-/cancer therapy, Werner syndrome, Bloom syndrome, De Barsy syndrome, Rothmund-Thomson syndrome, Cockayne syndrome, xeroderma pigmentosum, trichothiodystrophy, combined xeroderma pigmentosum-Cockayne syndrome, restrictive dermopathy, Wiedemann-Rautenstrauch syndrome, Hutchinson-Gilford progeria syndrome (progeria), a laminopathy, Ataxia telangiectasia-like disorder 2, XFE progeroid syndrome, Muscular dystrophy, Muscular Dystrophy (Becker's, Duchenne, Limb-Girdle), Yamamoto's Muscular Dystrophy, Mandibuloacral dysplasia, Dilated cardiomyopathy, GAPO syndrome, Cutis laxia, Ehlers-Danlos syndrome, Lenz-Majewski hyperostatic dwarfism, SHORT syndrome, Progressive external opthalmoplegia, Nester-Guillermo progeria syndrome, MDPL syndrome, Dyskeratosis congenital, Down syndrome.

### Sequence Variants

In one or more of Claims 1-58, “sequence variant” has ≥z % sequence identity, wherein z is a number between 27.9 and 100, for example selected from a group comprising 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5.

In one or more of Claims 1-58, “sequence variant” has ≥28% sequence identity.

In one or more of Claims 1-58, “sequence variant” has ≥30% sequence identity.

In one or more of Claims 1-58, “sequence variant” has >40% sequence identity.

In one or more of Claims 1-58, “sequence variant” has ≥50% sequence identity.

In one or more of Claims 1-58, “sequence variant” has ≥60% sequence identity.

In one or more of Claims 1-58, “sequence variant” has >70% sequence identity.

In one or more of Claims 1-58, “sequence variant” has >75% sequence identity.

In one or more of Claims 1-58, “sequence variant” has ≥80% sequence identity.

In one or more of Claims 1-58, “sequence variant” has ≥85% sequence identity.

In one or more of Claims 1-58, “sequence variant” has ≥90% sequence identity.

In one or more of Claims 1-58, “sequence variant” has ≥95% sequence identity.

In one or more of Claims 1-58, “sequence variant” has ≥96% sequence identity.

In one or more of Claims 1-58, “sequence variant” has >97% sequence identity.

In one or more of Claims 1-58, “sequence variant” has ≥98% sequence identity.

In one or more of Claims 1-58, “sequence variant” has ≥99% sequence identity.

In one or more of Claims 1-74, where a “sequence variant”, which is an amino acid sequence, is referred to, this is functional i.e. it (and/or some fragment(s) of it) can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater inhibitory activity against F₁F₀ ATP hydrolysis than a naturally occurring IF1 protein at pH 8;
and/or where a “sequence variant”, which is a polynucleotide sequence, is referred to, it codes for a functional amino acid sequence variant of the present claim: aforementioned in the present claim;
optionally, where “sequence variant” is written in Claims 1-74, this is substituted with “functional sequence variant”.

In one or more of Claims 1-75, where a “fragment” (or sequence variant of a fragment), which is an amino acid sequence, is referred to, this is functional i.e. it (and/or some fragment(s) of it) can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater F₁F₀ ATP hydrolysis inhibitory activity than a naturally occurring IF1 protein at pH 8.

### Scope

Where an amino acid/nucleotide sequence is presented, and/or referred to, herein, in alternative embodiments, this presentation/referral is of/to one or more of a “sequence variant” thereof, a fragment thereof (or concatenated fragments thereof), a “sequence variant” of a fragment thereof (or a “sequence variant” of concatenated fragments thereof).

### Sequence Variant Thereof

For an amino acid/nucleotide sequence of/in this disclosure, where the term “sequence variant”/“variant” is used/applies in relation to it, in some different (non-limiting) embodiments:
“sequence variant” has ≥28% sequence identity to the sequence;
“sequence variant” has ≥30% sequence identity to the sequence;
“sequence variant” has >40% sequence identity to the sequence;
“sequence variant” has ≥50% sequence identity to the sequence;
“sequence variant” has ≥60% sequence identity to the sequence;
“sequence variant” has ≥70% sequence identity to the sequence;
“sequence variant” has ≥75% sequence identity to the sequence;
“sequence variant” has ≥80% sequence identity to the sequence;
“sequence variant” has ≥85% sequence identity to the sequence;
“sequence variant” has ≥90% sequence identity to the sequence;
“sequence variant” has ≥95% sequence identity to the sequence;
“sequence variant” has ≥96% sequence identity to the sequence; “sequence variant” has ≥97% sequence identity to the sequence; “sequence variant” has ≥98% sequence identity to the sequence; “sequence variant” has ≥99% sequence identity to the sequence;
“sequence variant” has >z % sequence identity to the sequence, wherein z is a number between 27.9 and 100, for example selected from a group comprising 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 70.5, 71, 71.5, 72, 72.5, 73, 73.5, 74, 74.5, 75, 75.5, 76, 76.5, 77, 77.5, 78, 78.5, 79, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5;
where a “sequence variant”, which is an amino acid sequence, is referred to herein, in preferred embodiments this is functional i.e. it (and/or some fragment(s) of it) can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater F₁F₀ ATP hydrolysis inhibitory activity than a naturally occurring IF1 protein at pH 8;
where a “sequence variant”, which is a polynucleotide sequence, is referred to herein, in preferred embodiments it codes for a functional amino acid sequence, as referred to immediately above;
where “sequence variant” is written in this disclosure, in preferred embodiments this is substituted with “functional sequence variant”;
an esterified (on one or more carboxyl groups) derivative of a peptide/protein of this disclosure can be categorized within the scope of a “sequence variant”, because the amino acid sequence is changed, all be it reversibly because the sequence reverts after the peptide/protein passes into a cell and esterases cleave off the group(s), joined to one or more carboxyl groups, by ester bond(s).

### Fragment Thereof

For an amino acid sequence of/in this disclosure, where the term fragment (or sequence variant of a fragment) is used/applies in relation to it,
in some preferred embodiments this is functional i.e. it (and/or some fragment(s) of it) can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater F₁F₀ ATP hydrolysis inhibitory activity than a naturally occurring IF1 protein at pH 8;
in some embodiments, a fragment of a sequence refers to a shorter sequence by the removal of one or more amino acids, wherein these can be removed from one or both ends and/or at one or more internal places in the sequence.

### Cell Penetrating Peptides (CPPs)

For the CPP component of a fusion protein of this disclosure, the incorporation of any CPP(s) of the art is contemplated by, and componentry to, this disclosure. There are many CPPs of the art: some of which, >1,800, are disclosed in and its associated online database [“CPPsite”, presently at https://webs.iiitd.edu.in/raghava/cppsite].

Tat (U.S. Pat. Nos. 5,670,617, 5,747,641, 5,804,604, 5,674,980, 6,316,003B1) as the selected CPP, bound to the cargo peptide/protein by a peptide bond, can transport an IF1 protein sequence, or sub-sequence thereof, into cells (US2004/0072739A1, US2003/0026781A1, therein Tat is used with flanking glycine residues on one or both sides of the Tat sequence, as also used in [198]). Use of Tat as the CPP is one of the preferred embodiments herein. Tat can transport large proteins into cells, but in some preferred embodiments it is used to transport a functional IF1 protein fragment instead of a complete IF1 protein (non-limiting e.g. one of 1-60, 10-60, 10-47, 14-47, 14-46, 14-45, 14-44, 14-43, 14-42 or 42-58 IF1 protein residues, “mature” [without MIS] bovine IF1 protein numbering, or equivalent/aligned residues of a different IF1 protein from a different species). Incidentally, “TAT fusion peptides with cargos of <50 amino acids were rapidly taken up (within 3-5 min) and distributed throughout the cell, whereas TAT fusion proteins of >50 amino acids were mainly endocytosed and trapped largely in cytoplasmic vesicles” [199]. TAT₄₇₋₆₀ is the full Tat sequence, but there are shorter truncated versions known to those of the art. A notable Tat version ends in two proline residues (PP), which is good to have at the end of a CPP sequence, at the point of peptide bonded connection to the cargo sequence, for flexibility between these two domains.

A preferred CPP to use, especially in humans, is a CPP that has already been safely administered to one or more humans: preferably wherein it was used to carry a cargo sequence/compound, preferably as part of a licensed (e.g. FDA/EMA) drug and/or during a clinical trial (ideally wherein it passed at least Phase I trials). For non-limiting example, fulfilling the clinical trial aspect, is R7 (linked to cargo sequence by a pH sensitive linker [192], U.S. Pat. No. 6,730,293B1, applied to skin, Phase IIb discontinued because the pH sensitive linker didn't release the cargo as desired: “release of the free drug was not rapid enough to compete with clearance” [200], but it did pass Phase I trials for safety, and could passage the skin). Also, Tat (linked to PKC inhibitor cargo by disulphide bond, via added cysteine, —C—YGRKKRRQRRR [SEQ ID NO:449], in KAI-9803, Phase II completed, [201-202], U.S. Pat. No. 7,393,835B2, U.S. Pat. No. 7,507,711B2, U.S. Pat. No. 7,833,984B2), retroinverse Tat (bound by peptide bond in D-JNKI-1 [AM-111], which is in Phase III, [203], cargo-pprrrqrrkkrg [lower case denoting D-amino acid], U.S. Pat. No. 8,278,413B2, U.S. Pat. No. 8,183,339B1, U.S. Pat. No. 8,080,517B2) and PTD4 (in AXZ100, which has completed Phase IIa trials, [204], U.S. Pat. No. 8,974,774B2, U.S. Pat. No. 9,211,248B2).

R7 has the added advantage that it also corresponds to a sequence found within the human {and mouse} proteome (R7 sequence is found within a number of human proteins, findable by using BLAST). So, in humans, R7 is more likely to be safe, with less immunogenicity, than a sequence not found in humans. It also has the marketability, e.g. to a regulatory body and/or the public (e.g. when componentry to a cosmetic for slowing skin aging), of being a “natural” sequence (especially when attached to a human IF1 protein sequence, which is also a “natural” sequence, or a fragment/derivative thereof; or, in fact, to an IF1 protein sequence from another species, non-limiting e.g. bowhead whale). Penetratin also corresponds to a sequence found in humans (in a number of homeobox proteins). But it hasn't been used in humans to date. R7 has the further advantage that it confers superior cellular penetration than Tat or penetratin (e.g. when each is attached to the cargo sequence by introduced cysteines and a disulphide bond—twice as good [205], U.S. Pat. No. 6,730,293B1), wherein some longer poly-Arginine sequences than R7 can penetrate cells better [205], and some (e.g. R12) can be found within one or more human proteins. A poly-Arginine component to a fusion protein can be used to purify said fusion protein [207], wherein the fusion protein can be released from the immobilized negative resin by raising the pH sufficiently such that arginine loses its positive charge. To note, wherein amino acid count is in brackets, R7(7) is a shorter sequence than Tat (9 to 13) or penetratin (16). In [208], retroinverse penetratin has been associated with some cellular toxicity, but not the retroinverse of Tat, wherein the authors of that paper speculate this might be because Tat is shorter, and the toxicity of a retroinverse sequence correlates with its length (their speculation). Retroinverse penetratin (a variant with the amino acid(s) that have a chiral centre in their side chain swopped for another without this property) has greater cellular penetration than penetratin [209], which is likely a function of greater stability to protease action, because of its componentry D-, instead of L-, amino acids. The use of penetratin, and/or a penetratin variant(s)/derivative(s), e.g. from U.S. Pat. No. 6,992,169B2, with a cargo sequence herein, optionally wherein the cargo sequence is shorter than 100 amino acids, is hereby contemplated.

A preferred CPP to use is R7 (is clinically validated), or other poly-Arginine (or polymer of arginine and/or one or more arginine analogues) longer than 5 residues, preferably shorter than 50 residues, optionally less than 16 residues, attached to the cargo sequence by a peptide or disulphide bond, the latter courtesy of a cysteine introduced into the CPP and another cysteine introduced into, or already in, the cargo sequence ([205], U.S. Pat. No. 7,393,835B2, U.S. Pat. No. 7,507,711B2, U.S. Pat. No. 7,833,984B2, U.S. Pat. No. 6,730,293B1), optionally wherein one or more arginine residues are D-amino acids, optionally at least 50% of them, optionally all of them, optionally wherein the sequence (just the CPP and/or the cargo sequence) is retroinverse. For example, RRRRRRRC-C [cargo] or rrrrrrrC-C [cargo] or rrrrrrrc-c [cargo], i.e. SEQ ID NO:459 concatenated to [cargo], where lower case denotes D-amino acid. When R7, or other favoured poly-Arginine, is bound to the cargo sequence by a peptide bond, a linker/spacer sequence between, optionally comprising one or more (preferably less than 5, ideally 1 to 2) glycine and/or proline residues, optionally a cleavable linker sequence (e.g. cleavable by a ubiquitous enzyme(s) such as an esterase {e.g. a linker containing a carboxylic acid ester}, amidase, or the like, working to the assumption that the concentration of such an enzyme(s) is higher inside than outside cells in the extracellular milieu, refer U.S. Pat. No. 9,255,124B2 for more on such linkers, pH dependent and other linkers in U.S. Pat. No. 6,730,293B1), is componentry to this disclosure. If one of the art cannot work a most/more favoured option (e.g. R7) they should experiment with another option(s) herein (e.g. R7 with one or more flanking glycines, e.g. a Tat sequence e.g. a Tat sequence with flanking glycines e.g. YGRKKRRQRRRG [SEQ ID NO:446] or GYGRKKRRQRRRG [SEQ ID NO:445]). Notably, R7 followed by a glycine, RRRRRRRG [SEQ ID NO:461], is a sequence found within the human (and mouse) proteome (e.g. in tight junction protein ZO-1 isoform X1 in human), and so can in some sense be said to be a “natural” sequence in humans, whilst its R7 component has clinical safety precedent, and this is a preferred CPP sequence to use in this disclosure. R7 followed by a proline is also found within the human (and mouse) proteome (e.g. in alpha adrenergic receptor subtype alpha 1a in human) and is another preferred CPP sequence. Longer poly-arginine sequences (up to R50), flanked on one or both sides with one or more glycine and/or proline residues, are also contemplated for use as a CPP sequence herein.

More broadly, the most favoured CPPs to use, especially when administering to a human subject(s), are, in decreasing order of preference, poly-Arginine (preferably, where n is the number of arginine residues, n≥5, more preferably n≥6, more preferably 50≥n≥6, optionally 15≥n≥6, optionally R7 or R8 or R9, and/or variant thereof) and/or Tat (and/or variant thereof) and/or penetratin (and/or variant thereof), attached to the cargo by peptide/ester bond (for example ester bonded to the C-terminus), or a disulphide bond conferred by a cysteine introduced into the CPP sequence (preferably at its N- or C-terminal ends) and a cysteine naturally in (or introduced/substituted into) the cargo sequence (optionally at its N- or C-terminal ends), wherein the aforementioned variants can (for non-limiting example) be the sequence in reverse, and/or have one or more D in place of L-amino acids, or be the retroinverse sequence, and/or have one or more (up to 10, preferably less than 5, more preferably no more than 3) flanking glycine and/or proline residues on one or both ends. Variants with D-instead of L-amino acids tend to have greater stability, being less susceptible to proteases, and so confer greater penetration and cargo delivery into cells [when serum is present] ([206], U.S. Pat. No. 6,730,293B1).

When the CPP is attached to the cargo sequence by a disulphide bond, this breaks within the reducing intracellular environment, releasing the cargo after the CPP has passaged it across the plasma membrane. When the CPP is attached by a peptide bond to a Mitochondrial Import Sequence (MIS), connected to an IF1 protein/fragment (or sequence variant thereof), then the CPP directs this fusion protein across the plasma membrane, the MIS then directs this fusion protein into the mitochondrial matrix, thereafter the MIS is enzymatically cleaved (and the CPP at its N-terminal end with it), leaving the IF1 protein/fragment (or sequence variant thereof) unencumbered inside the mitochondrial matrix. The latter, linkage by peptide bond, is more favoured than linkage by disulphide bond because it doesn't have the problem of “disulfide bond exchange” (U.S. Pat. No. 9,255,124B2), wherein a disulphide bond between CPP and cargo is replaced by a disulphide bonds between two CPP sequences and two cargo sequences, wherein these cargo sequences, without a CPP, don't have the capacity to cross a plasma membrane, and the amount of conjoined (effective) CPP-cargo in the sample is reduced. What can reduce this effect, if not already present, is to insert at least one aliphatic residue (e.g. alanine, valine, leucine, isoleucine) next to one or both of the cysteines, on one or (if the cysteine is not at an N- or C-terminal end) both sides, and wherever a cysteine is specified in a sequence herein, and is involved in a disulphide bond, in further embodiments, at least one aliphatic residue is inserted/substituted as aforementioned. Notably a cysteine that is internal to a sequence, instead of at the N- or C-terminal ends, is less likely to be affected by this “disulfide bond exchange” effect, and if both cysteine residues in the disulphide bond are internal to their sequence, the chance is lower still, and this effect can be reduced further by including one or more excipients disclosed in U.S. Pat. No. 7,265,092B2 in the pharmaceutical composition (e.g. mannitol).

Administration via skin is contemplated, wherein Tat, R7, penetratin, and some other CPPs, have been shown to confer penetration of a cargo sequence through skin [210]. Optionally, when administered by skin, the skin is microporated (US20100311671A1). Protein/peptide conjugation with a fatty acid, palmitic acid (palmitoylation) or similar, which helps peptides go through the skin barrier, is contemplated. Administration to skin is especially good for local/constrained administration to that part of skin, without much systemic exposure at all, because many of these cell-permeable fusion proteins herein, when administered by skin, don't travel too far into the body before arriving in conditions that cause them to release their cargo, wherein the cargo, when it is an IF1 protein/fragment (or sequence variant thereof), cannot pass a plasma membrane after its CPP element has dropped it off. Administration to the eye is also contemplated, wherein a poly-Arginine CPP can deliver a cargo peptide to the eye, especially with an optimized pharmaceutical composition for eye delivery (e.g. refer U.S. Pat. No. 8,729,010B2 and references therein).

If desired, greater penetration of these fusion proteins, for example into the skin, can be conferred by using an “activatable CPP”, wherein the fusion protein contains a polyanionic sequence that prohibits cellular entry conferred by its CPP (which can, for example, be a poly-Arginine sequence, e.g. R8), wherein this “anchor domain”, preventing cellular entry, can be cleaved off by some external stimuli, e.g. by the action of an extracellular protease(s). This approach has some precedent, including in the clinic: [211-213], U.S. Pat. No. 9,695,251B2, U.S. Pat. No. 7,985,401B2, U.S. Pat. No. 10,385,380B2, U.S. Pat. No. 10,596,259B2, US2006/0041105A1, US2012/0134922A1, US2015/0359902A1, WO2005/042034A1, WO2011/008996A2.

In some embodiments, a poly-arginine that has L- and D-amino acids alternate in the sequence is used as the CPP e.g. RrRrRrR [SEQ ID NO:460], where lower case denotes D-amino acid. In other embodiments, the used CPP is a string of L- and/or D-arginine residues, up to 50 residues long, optionally less than 16 amino acids, punctuated at one or more points, and/or flanked on one or both sides, with one or more independently selected L- and/or D-hydrophobic amino acids, each preferably with a hydrophobicity equal or greater than phenylalanine, even more preferably with a hydrophobicity equal or greater than cyclohexylalanine. In particular embodiments, one or more L- or D-arginine residues (e.g. a string of 7) alternates with one or more independently selected hydrophobic amino acids, for 1-30 repeats.

The use of a lipidated CPP is contemplated. For (non-limiting) example, a poly-Arginine CPP (e.g. R7 or R8) with a fatty acid (saturated or unsaturated, straight-chain or branched, with between 2 and 100 carbon atoms, preferably with between 2 and 25 carbon atoms) acylated to its N-terminal end [214-215]. In some embodiments, if a fusion protein of/in this disclosure, contains at least one CPP sequence, a fatty acid moiety can be conjoined to one or more amino acid side-chains in the CPP sequence, preferably to just one, optionally to a present/introduced lysine residue (e.g. acylated to its side-chain), and/or, if this CPP sequence is at the N-terminal end of the fusion protein, a fatty acid moiety can be conjoined/acylated to its N-terminus.

The table below presents some non-limiting examples of Cell Penetrating Peptides (CPPs) of the art [upper case letter reflects L-amino acid, lower case reflects D-amino acid; any presented L-amino acid or D-amino acid is, in other embodiments, contemplated to be the other, all possible L- and D-amino acid combinations are contemplated, the reverse sequences also contemplated, and the retroinverse sequences]. Use of one or more of these (and/or of another(s) of the art), as taught herein for a CPP, is componentry to this disclosure. Each can be attached at the N- or C-terminus of the cargo protein/peptide in either orientation (forward or reverse). Optionally attached via a covalent (preferably peptide) bond, optionally via a linker/spacer sequence, optionally comprising one or more glycine and/or proline residues, or by a cysteine pair, between the CPP and cargo sequences, linked by a disulphide bond (e.g. refer linker in WO2018232491A1), or by a photocleavable (U.S. Pat. No. 8,729,010B2) or pH sensitive linker (U.S. Pat. No. 6,730,293B1,) [192]) or “self-immolative” (U.S. Pat. No. 10,624,968B2) linker or protease(s) sensitive linker, or connected in some other way (non-limiting e.g. further linkers given in U.S. Pat. No. 8,729,010B2 and references therein). Optionally, via a cysteine residue added at the N- or C-terminus of a CPP sequence, which can be attached by a disulphide bond to a cysteine (optionally inserted/substituted) at the N- or C-terminus, or within, the cargo sequence. A sub-sequence of a CPP sequence and/or reverse sequence of a CPP sequence and/or a CPP sequence with one or more constituent L-amino acids replaced with their corresponding D-amino acids (reduces susceptibility to proteases e.g. in the blood) and/or the retroinverse sequence of a CPP sequence and/or a CPP sequence with modification at its N- or C-terminal ends (preferably a modification known to those of the art, non-limiting e.g. acylation (e.g. acetylation) at the N-terminus, amidation at the C-terminus, cholesteryl moiety attached by an ester bond to C-terminus [e.g. as in [209]], modifications that remove charge and/or increase lipophibicity are especially favoured), in use as a CPP sequence, is hereby contemplated. When a retroinverse (part[s] or entirety) sequence is used, optionally amino acids with a chiral centre in their side-chain (isoleucine and threonine) are replaced with an amino acid without this feature (e.g. valine), and/or proline residues are removed or replaced with a different amino acid [209]. In the table below is such a retroinverse sequence of Penetratin (with I replaced with V). The use of a CPP of 5 to 51 amino acids, of which at least 50% are arginine, optionally/preferably comprising one or more regions of 5 or more contiguous arginine residues, is componentry to this disclosure. By the teaching of this disclosure, further CPP sequences can be found by searching for very arginine rich motifs within proteins, preferably from human. Some further CPPs for use as part of this disclosure can be found in one or more of US2008/0234183A1, US2014/0140929A1, WO 01/96369A1, U.S. Pat. No. 10,287,331, US2008/0234183A1, US2014/0140929A1, US2003/0032593A1, WO97/12912, WO2007/108749A1, U.S. Pat. No. 8,974,774B2, U.S. Pat. No. 9,211,248B2, U.S. Pat. No. 7,049,286B2, U.S. Pat. No. 8,410,045B2, WO2009/036092A2, U.S. Pat. No. 10,293,020B2, WO2013/086020A1, U.S. Pat. No. 10,287,331B2, WO2007108749A1, U.S. Pat. No. 10,626,147B2 or a cited/citing/related paper/patent or application thereof, or in the “CPPsite” database ([197]) of CPPs at https://webs.iiitd.edu.in/raghava/cppsite, or elsewhere in the literature. Cyclic CPPs are herein contemplated e.g. refer to U.S. Pat. No. 10,626,147B2, US 2019/0309020A1, US2017/0355730A1. Tat as used herein typically refers to that found in HIV-1. But the use of a Tat sequence from a different virus is also contemplated, non-limiting examples of which include VP22 from HSV (WO97/05265; Elliott and O'Hare, Cell 88:223-233, 1997), HIV-2 (M. Guyader et al., “Genome Organization and Transactivation of the Human Immunodeficiency Virus Type 2”, Nature, 326, pp. 662-669(1987)), equine infectious anemia virus (R. Carroll et al., “Identification of Lentivirus TAT Functional Domains Through Generation of Equine Infectious Anemia Virus/Human Immunodeficiency Virus Type 1 TAT Gene Chimeras”, J. Virol., 65, pp. 3460-67(1991)), and simian immunodeficiency virus (L. Chakrabarti et al., “Sequence of Simian Immunodeficiency Virus from Macaque and Its Relationship to Other Human and Simian Retroviruses”, Nature, 328, pp. 543-47(1987); S. K. Arya et al., “New Human and Simian HIV-Related Retroviruses Possess Functional Transactivator (tat) Gene”, Nature, 328, pp. 548-550(1987)). The use of multiple CPPs, which can all be the same (e.g. dimer, trimer etc.) or be more than one type, is herein contemplated. Indeed, this has clinical precedent in the macromolecule transport system (MTS), comprising a positively charged lysine-rich central domain inbetween two TAT49-57 domains, enabling large molecules to cross the skin, used by Revance Therapeutics Inc. to get botulinum toxin type A into human skin in clinical trials (U.S. Pat. No. 9,211,248B2, US2007/0077259A1, US2008/0038203A1, US2010/0093639A1). Multimers with a plurality of CPP sequences and/or a plurality of cargo sequences are contemplated. The use of any Diatos Peptide Vector (DPV)/VectoCell® (e.g. U.S. Pat. No. 8,410,045B2) as the CPP element of a fusion protein of this disclosure is herein contemplated. When using a fusion protein of this disclosure for anti-cancer activity, in some embodiments its selected CPP element has intrinsic anti-cancer utility in and of itself e.g. p28 [216], which is presently in clinical trials for anti-cancer use. Using CAMP as the CPP confers both a CPP and a Mitochondrial Import Sequence (MIS), negating the need for a MIS. For mitochondrial targeting, use of the MIS component of CAMP (LLRAALRKAAL fragment of SEQ ID NO:452) with a different CPP (e.g. polyarginine e.g. R7) in fusion proteins of this disclosure is contemplated, wherein this MIS is advantageously short (11 residues). Some sequences with high sequence identity to a known CPP are also likely to be a CPP, and their use (one or more) as a CPP in fusion proteins of this disclosure is contemplated. Any amino acid sequence/chemical moiety (or plural thereof) that can transport a cargo amino acid/nucleotide sequence across a cell membrane can be incorporated as the CPP component of a fusion protein of this disclosure. In alternative fusion protein embodiments of this disclosure, at least one Cell-Penetrating poly(Disulfide) (CPD) moiety and/or Skin Penetrating Peptide (SPP, e.g. one or more SPPs found in one or more of U.S. Pat. No. 7,659,252B2, U.S. Pat. No. 8,791,062B2, U.S. Pat. No. 9,642,895B2, US2014/0161871A1, US2014/0227174A1, US2015/0025221A1, US2017/0258930A1, WO2007/035474A2, WO2014/123543A2, WO2016/033314A1, WO2017/048812A1, and/or in patents/patent applications that cite them, or that they cite) is used instead of, or in addition to, at least one CPP moiety.

| | SEQ | |
|---|---|---|
| | ID | |
| CPP | NO: | Sequence |
| TAT₄₇₋₆₀ | 440 | YGRKKRRQRRRPPQ |
| TAT₄₈₋₆₀ | 441 | GRKKRRQRRRPPQ |
| TAT₄₇₋₅₇ | 442 | YGRKKRRQRRR |
| TAT₄₈₋₅₇ | 443 | GRKKRRQRRR |
| TAT₄₉₋₅₇ | 444 | RKKRRQRRR |
| Flanking G | 445 | GYGRKKRRQRRRG |
| | 446 | YGRKKRRQRRRG |
| | 447 | GRKKRRQRRRPP |
| | 448 | YGRKKRRQRRRPP |
| | 449 | CYGRKKRRQRRR |
| | 450 | CRKKRRQRRR |
| | 451 | pprrrqrrkkrg |
| CAMP | 452 | YGRKKRRQRRRLLRAALRKAAL |
| R9-TAT | 453 | GRRRRRRRRRPPQ |
| PTD4 | 454 | YARAAARQARA |
| R7 | 455 | RRRRRRR |
| R8 | 456 | RRRRRRRR |
| R11 | 457 | RRRRRRRRRRR |
| | 458 | RRRRRRRC |
| | 459 | RRRRRRRCC |
| | 460 | RrRrRrR |
| Flanking G | 461 | RRRRRRRG |
| R6W3 | 462 | RRWWRRWRR |
| Penetratin | 463 | RQIKIWFQNRRMKWKK |
| | 464 | RRMKWKK |
| | 465 | NRRMKWKK |
| | 466 | QNRRMKWKK |
| | 467 | CRQIKIWFQNRRMKWKK |
| | 468 | Kkwkmrrnqfwvkvqr |
| VP22 | 469 | DAATATRGRSAASRPTERPRAPARSASRPRRPWD |
| DPV3 | 470 | RKKRRRESRKKRRRES |
| DPV6 | 471 | GRPRESGKKRKRKRLKP |
| DPV1047 | 472 | CVKRGLKLRHVRPRVTRMDV |
| DPV1047 | 473 | VKRGLKLRHVRPRVTRMDV |
| pVEC | 474 | LLIILRRRIRKQAHAHSK |
| ARF(1-22) | 475 | MVRRELVTLRIRRACGPPRVRV |
| ARF(19-31) | 476 | RVRVFVVHIPRLT |
| MPG | 477 | GALFLGFLGAAGSTMGAWSQPKKKRKV |
| MAP | 478 | KLALKLALKALKAALKLA |
| Transportan | 479 | GWTLNSAGYLLGKINLKALAALAKKIL |
| Transportan | 480 | AGYLIGKINLKALIAALAKKIL |
| 10 | | |
| | 481 | GWTLNSAGYLLG |
| | 482 | INLKALIAALAKKIL |
| Bip4 | 483 | VSALK |
| C105Y | 484 | CSIPPEVKENPFVYLI |
| Melittin | 485 | GIGAVLKVLTTGLPALISWIKRKRQQ |
| gH625 | 486 | HGLASTLTRWAHYNALIRAF |
| Pep-1 | 487 | KETWWETWWTEWSQPKKKRKV |
| Pep-7 | 488 | SDLWEMMMVSLACQY |
| BPrPr(1-28) | 489 | MVKSKIGSWILVLFVAMWSDVGLCKKRP |
| p28 | 490 | LSTAADMQGVVTDGMASGLDKDYLKPDD |
| VT5 | 491 | DPKGDPKGVTVTVTVTVTGKGDPKPD |
| Bac 7 | 492 | RRIRPRPPRLPRPRPRPLPFPRPG |
| (Bac₁₋₂₄) | | |
| PFVYLI | 493 | PFVYLI |
| NLS | 494 | CGYGPKKKRKVGG |
| KALA | 495 | WEAKLAKALAKALAKHLAKALAKALKACEA |
| P1 | 496 | MGLGLHLLVLAAALQGAWSQPKKKRKV |
| hCT | 497 | LGTYTQDENKFHTFPQTAIGVGAP |
| SynB1 | 498 | RGGRLSYSRRRESTSTGR |
| SynB3 | 499 | RRLSYSRRRF |
| SynB4 | 500 | AWSFRWSYRGISYRRSR |
| SW-40 (NLS) | 501 | PKKKRKW |
| SAP | 502 | WRLPPPWRLPPPWRLPPP |
| pISL | 503 | RVIRVWFQNKRCKDKK |
| GALA | 504 | WEAAIAEALAEALAEHLAEALAEALEALAA |
| N50 | 505 | WQRKRQKLM |
| pISL | 506 | RVIRVWFQNKRCKDKK |
| VT5 | 507 | DPKGDPKGWTWTVTWTVTGKGDPKPD |
| HN-1 | 508 | TSPLNIHNGQKL |
| Ku70 | 509 | WPMILKPMLKE |
| Buforin II | 510 | TRSSRAGLQFPWGRVHRLLRK |
| hCT (9-32) | 511 | LGTYTQDFNKFHTFPQTAIGVGAP |

### CPPs Comprising (Predominantly/Completely) Positively-Charged and Hydrophobic Residues

Any CPP comprising a number of positively charged residues, preferably arginine, wherein the majority, or all, the other residues are hydrophobic residues. Without seeking restriction by theory, the concentration of positive charges, particularly if due to arginine residues, confers cellular entry (into the negative inside cytoplasm), and the hydrophobic residues facilitate this. Wherein given that the mitochondrial inner membrane has a large negative inside (mitochondrial matrix) membrane potential across it (around −140 mV in normal cells, more hyperpolarized in cancer cells), a CPP with this character can tend to disproportionally accumulate its cargo in the mitochondrial matrix. So, such CPPs are sometimes termed Mitochondria Penetrating Peptides (MPPs) in the literature. Mitochondrial matrix localization of cargo is highly desirable in the use cases of this disclosure. The incorporation of any MPP (as a CPP) in a fusion protein of this disclosure is hereby contemplated. In some cases, the use of an MPP as the CPP can negate the need for a Mitochondrial Import Sequence (MIS) because the MPP confers not just cellular entry, but mitochondrial matrix localization also. Although use of MIS and MPP/CPP has the advantage that there is an enzyme that cleaves off the MIS (from the cargo sequence) in the mitochondrial matrix, and so if the MPP/CPP is N-terminal to the MIS, and the cargo C-terminal to the MIS, then the MPP/CPP is inherently cleaved off along with the MIS, and so the cargo is then unencumbered at its target destination. The use of a CPP with multiple positive charges, preferably 3, and more preferably 5 or more, preferably due to arginine residues, interspersed with (and/or adjacent to) one or more hydrophobic amino acid residues, optionally cyclohexylalanine (and/or other amino acid with an equivalent or greater log P), such that the log P of the CPP is greater than-3, and more preferably greater than-2.5, optionally wherein the CPP comprises a charge of +3 and a log P value of at least about-1.7, optionally wherein the CPP comprises a charge of +5 and a log P value of at least about-2.5, preferably wherein the CPP is less than 12 amino acids long, is componentry to this disclosure, wherein these characteristics tend to confer mitochondrial localization to the CPP and its cargo: as observed in [218-219] and U.S. Pat. No. 9,132,198B2 {and the papers/patents/patent applications that cite these}, wherein all MPP/CPP sequences therein are incorporated by reference into this disclosure).

Any CPP comprising a number of positively charged residues, preferably arginine, wherein the majority, or all, the other residues are hydrophobic residues except for a single glutamine (Q) residue, which is preferably located at an end of the sequence. CPP comprising (or consisting of) 3, 4, or 5 (or more) arginine residues, 2 or 3 (or more) hydrophobic residues, and a glutamine residue (preferably at an end of the sequence); in alternative embodiments, a plurality of glutamine residues (e.g. 2 or 3) is contemplated; in more preferred embodiments, no glutamine residue is present. Some CPP sequences in the literature have glutamine in them (e.g. [220-221]). But I think this is incorporated just to enable the conjugation of a dye, such that CPP intracellular localization can be tracked, rather than being an important component to the CPP sequence. Glutamine is not positively charged and is not hydrophobic. And so, by the teaching herein, is a drag to CPP performance, and so should be cut out of these CPP sequences to confer better CPP sequences.

SEQ ID NO:512 encompasses some CPP/MPP sequences of this disclosure (optionally with flanking glycine and/or proline residue[s] on one or both sides). In SEQ ID NO:512, Xaa, at each position within the residue range 3-22, is independently selected from: L-arginine, D-arginine, L-Lysine, D-lysine, L-phenylalanine, D-phenylalanine, L-tryptophan, D-tryptophan, L-tyrosine, D-tyrosine, L-glutamine, D-glutamine, L-phenylglycine, D-phenylglycine, 3,3-diphenyl-L-alanine, 3,3-diphenyl-D-alanine, L-3-cyclohexylalanine, D-3-cyclohexylalanine, L-3-(1-naphthyl) alanine, D-3-(1-naphthyl) alanine, L-3-(2-naphthyl) alanine, D-3-(2-naphthyl) alanine, L-2-aminooctanoic acid, D-2-aminooctanoic acid, O-Methyl-L-tyrosine, O-Methyl-D-tyrosine, 2,6-dimethyl-L-tyrosine, 2,6-dimethyl-D-tyrosine, or absent. In further embodiments, Xaa at each position within the residue range 3-22 of SEQ ID NO:512 is independently selected from its aforementioned options, but also from L-2,4,6-trimethylphenylalanine, D-2,4,6-trimethylphenylalanine, 2-cyclohexyl-L-glycine, 2-cyclohexyl-D-glycine, 3-benzo[b]thiophen-3-yl-L-alanine, 3-benzo[b]thiophen-3-yl-D-alanine, L-homophenylalanine, D-homophenylalanine, 4-fluoro-L-phenylalanine, 4-fluoro-D-phenylalanine, 4-chloro-L-phenylalanine, 4-chloro-D-phenylalanine, 3,4-difluoro-L-phenylalanine, 3,4-difluoro-D-phenylalanine, wherein all the options can optionally have 1 to 4 substituents, independently selected from alkyl and halogen. In alternative embodiments, in addition, more types of hydrophobic amino acid can also be selected from. For example, other amino acids with one or more of cyclo, diphenyl, naphthyl, or hexyl components. Amino acids can be sourced from chemical suppliers listed on the website www.labnetwork.com or well-known (to those of the art) chemical stockists such as Sigma-Aldrich, or by the guidance of a peptide synthesis company (some examples of which are given elsewhere herein). In some embodiments, tryptophan incorporation is disfavoured because it can have a negative impact upon pharmaceutical stability. SEQ ID NO:513 to SEQ ID NO:520 encompass increasingly narrower sub-sets of the sequence space of SEQ ID NO:512, always maintaining arginine, but soon dropping lysine, and iteratively dismissing the least hydrophobic of the hydrophobic residue options. A distinctive option is SEQ ID NO:520, which distinctively only contains natural amino acids (and D forms thereof). Preferred sequences of any of SEQ ID NO:512 to SEQ ID NO:520 have multiple positively charged, preferably arginine, residues (optionally wherein these are concentrated i.e. close to one another e.g. adjacent, e.g. not many residues apart) and one or more hydrophobic residues, wherein for the latter, in some embodiments, more hydrophobic residues are favoured over less hydrophobic residues. Some preferred sequences within and beyond SEQ ID NO:512 are within SEQ ID NO:521 and SEQ ID NO:522.

Contemplated is any sequence of SEQ ID NO:512 to SEQ ID NO:522 that has one or more of its residues covalently bound by a lipidic moiety e.g. a fatty acid (or derivative thereof), e.g, wherein a fatty acid is acylated to the N-terminus and/or wherein a fatty acid is acylated/conjugated to one or more lysine side-chains, optionally/preferably wherein the fatty acid comprises/contains between 2 to 100 {or 2 to 25} carbon atoms, optionally being a myristoyl/palmitoyl/stearoyl group. A bound fatty acid(s) advantageously increases the sequence's hyrophibicity.

SEQ ID NO:523 to SEQ ID NO:638 presents some non-limiting example embodiments of SEQ ID NO:12. Wherein some of the sequences within SEQ ID NO:523 to SEQ ID NO:550 are found in U.S. Pat. No. 9,132,198B2 and/or [218-219]. Some of the sequences within SEQ ID NO:551 to SEQ ID NO:629 are found in one or more of U.S. Pat. No. 10,626,147B2, U.S. Pat. No. 10,501,496B2, US 2019/0309020A1, US20170190743A1, US20170355730A1, US2019/0282654A1, US2019/0284240A1, WO2015/179691A2, [220-221] (wherein they are shown adept at conferring cellular entry of cyclic/bicyclic proteins). SEQ ID NO:630 contains SS-31 which is known to locate to mitochondria and has been used safely in clinical trials. Any of SEQ ID NO:551 to SEQ ID NO:629 with glutamine in it, in alternative (more preferred) sequence embodiments, this glutamine is absent. Not shown, but contemplated, and componentry to this disclosure, is the sequence of any of SEQ ID NO:523 to SEQ ID NO:630 in reverse (or only part(s) thereof is in reverse) and/or wherein the same amino acids are present but they are in a different order within the sequence and/or wherein the stereochemistry of at least one amino acid is inverted from how it is presented in the Sequence Listing (i.e. L to D form, or D to L form), optionally wherein the stereochemistry of all amino acids is inverted, optionally wherein only those amino acids with L stereochemistry are inverted (to D form), and/or wherein one or more hydrophobic residues are substituted with a different hydrophobic residue (preferably a more hydrophobic residue) independently selected at each position from the following options in this sentence; any sequence selected from SEQ ID NO:523 to SEQ ID NO:630 wherein one or more hydrophobic residues within it are substituted with a different hydrophobic residue (preferably a more hydrophobic residue) independently selected at each position from the following options: L-phenylalanine, D-phenylalanine, L-tryptophan, D-tryptophan, 3,3-diphenyl-L-alanine, 3,3-diphenyl-D-alanine, L-3-cyclohexylalanine, D-3-cyclohexylalanine, L-3-(1-naphthyl) alanine, D-3-(1-naphthyl) alanine, L-3-(2-naphthyl) alanine, D-3-(2-naphthyl) alanine, L-2-aminooctanoic acid, D-2-aminooctanoic acid, O-Methyl-L-tyrosine, O-Methyl-D-tyrosine, 2,6-dimethyl-L-tyrosine, 2,6-dimethyl-D-tyrosine, 2-Cyclohexyl-L-glycine, 2-Cyclohexyl-D-glycine. SEQ ID NO:631 to SEQ ID NO:638 are some of the novel CPP sequences by the teaching of this disclosure.

Innovatively, I have developed SEQ ID NO:512 (and encompassed sequences thereof) which teaches further novel CPP sequences of this disclosure. Wherein, by the teaching of the present disclosure, the precise sequence isn't as important as having multiple positively charged, preferably arginine, residues and one or more hydrophobic residues. A greater number of positive charges drives lower sequence log P, whilst a greater number of hydrophobic residues drives greater log P, wherein some preferred sequences have multiple positive charges (preferably due to multiple arginine residues) with reasonably high log P, which aren't too long. Wherein a useful metric (denoted u) for a CPP sequence is its number of positive charges (preferably due to arginine residues) plus its log P value, optionally (in some embodiments of μ) divided by its number of residues or/and wherein the number of possible residues in the sequence is capped at a selected number (optionally wherein this number is in the range 3-20 or 3-15, or 3-10, or 3-7), wherein a relatively high value of u is desirable in some embodiments: for example, equal to or (more preferred) higher than that for one or more of SEQ ID NO:523 to SEQ ID NO:630. For example, without the aforementioned division step, in some embodiments, a desirable value of μ is greater than 1, and an even more desirable value is greater than 2, and higher values of μ are even more desirable. With the aforementioned division step, in some embodiments, a desirable value of μ is greater than 0.6, and an even more desirable value is greater than 0.8, and higher values of μ, especially those over 1, are even more desirable. So, in some embodiments, one can rank a number of sequences from SEQ ID NO:512 (or one of SEQ ID NO:513 to SEQ ID NO:522) by which has the greatest μ value, and optionally select the sequence to use on this basis (or use this as one factor in the selection criteria). When two sequences of different lengths have the same or nearly equal μ value, the shorter sequence is favoured. By reducing all this complexity to such simple arithmetic (simply summing the log P values of each amino acid, and any positive charge[s] [preferably due to arginine residue(s)], in each amino acid sequence of specifically limited sequence length and/or wherein the sum is divided by the number of residues), one of the art can see that this is algorithmically tractable. One of the art can also envisage more complicated mathematical schemes knowing now the aims. For example, wherein the penalty cost of another residue is greater than 1 and/or there is an increasing penalty cost the longer the sequence (i.e. cost [denominator] doesn't increase linearly with length as in the scheme described, but super-linearly) and/or wherein a minimum number of positive charges present in the sequence is set (i.e. the number of positive charges needs to be equal to, or exceed, this number) and/or wherein the value contributed by a positive charge is greater than 1 [thence weighting the value of the positive charges more against that of the log P consideration, wherein in some embodiments this is implemented] and/or wherein a positive charge contributed by an arginine residue is given greater weight than that contributed by a lysine (e.g, wherein that ascribed to lysine is a fraction of that ascribed to arginine). In some embodiments, log D is used instead of log P in the calculations. In some embodiments, the positively charged residues are concentrated in the sequence i.e. close to one another e.g. adjacent, e.g. not many residues apart. In some embodiments, D-form of natural amino acids are used.

In some embodiments, a favoured CPP has at least 3 or more, or 5 or more, positive charges (preferably some or more, more preferably all, of which are arginine conferred), which lowers sequence log P, and requires some hydrophobic residues to increase log P, wherein residues with greater hydrophibicity are favoured because less of them are required to appreciably increase log P, and thence their use permits a higher value of μ (when its sequence length penalty is used). So, in these embodiments, the favouring of which hydrophobic amino acids to incorporate is according to which are most hydrophobic, with the most hydrophobic being most favoured typically (notwithstanding some parallel consideration of which are safe to administer to mammals, wherein those with a record of safe use in humans {e.g. as part of a drug candidate that has passed at least Phase I trials, or a licensed drug} are prioritized). The ordering of log P of some hydrophobic amino acids (not including those with halogen atoms, which are also contemplated but which tend to be slightly disfavoured): from highest to lowest (calculated by MarvinSketch software [Chemaxon, Budapest, Hungary] [5]): 2,4,6-trimethylphenylalanine, 3,3-diphenyl-alanine, 3-(1-naphthyl) alanine (used in BT1718 from Bicycle Therapeutics, presently in Phase I clinical trials), 3-(2-naphthyl) alanine, 2,6-dimethyl-tyrosine (used in clinical drug candidate SS-31 [called Elamipretide], which has passed Phase I safety trials), 2-aminooctanoic acid, cyclohexylalanine, cyclohexylglycine, tryptophan, phenylalanine, O-methyl-tyrosine, phenylglycine. So, 3-(1-naphthyl) alanine and 2,6-dimethyl-tyrosine are some especially prized hydrophobic residues to incorporate into sequences of SEQ ID NO:512 (or one of SEQ ID NO:513 to SEQ ID NO:522) because they have high log P and clinical precedent. Some CPP sequence embodiments, only contain arginine (preferably at least 3, or at least 5 residues thereof) and 3-(1-naphthyl) alanine and/or 2,6-dimethyl-tyrosine residues, wherein the L- or D-form is independently selected for each residue position.

In some embodiments, a CPP of this disclosure comprises (or consists of) one or more arginine residues alternate/alternating with one or more hydrophobic residues, optionally wherein the hydrophobic residues are independently selected at each position from the aforementioned list, optionally for 1 to 20 repeats, optionally wherein the repeat size can be variable within the sequence, optionally with a glutamine residue at one end (but preferably without this), optionally the CPP sequence is represented by the following formula, wherein Q is L- or D-glutamine (preferably absent), R is arginine (L- or D-arginine independently selected at each position) and @ is a hydrophobic residue, independently selected in each case, preferably at each selection it is one of the aforementioned hydrophobic residues (or a D- or L-stereoisomer thereof):
(Q)₀₋₁{(R)₀₋₁₀(Φ)₀₋₁₀}₁₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀( )₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀{(R)₀₋₁₀(Φ)₀₋₁₀}₀₋₂₀(Q)₀₋₁

Preferably the above formula can be applied to select a sequence(s) from SEQ ID NO:512 (or at least one sequence within any of SEQ ID NO:513 to SEQ ID NO:522). In some CPP sequence embodiments, the number of positively charged (preferably arginine) residues and number of hydrophobic residues is equal, and in other embodiments, unequal, wherein the number of positively charged (preferably arginine) residues can be greater than the number of hydrophobic residues, or the inverse. In some embodiments, the number of hydrophobic residues is the number of positively charged (preferably arginine) residues plus 1 or 2 or 3 or 4, or minus 1 or 2 or 3 or 4. CPP comprising (or consisting of) a number of arginine residues selected from 3, 4, 5, 6, 7 (or more), and a number of hydrophobic residues selected from 2, 3, 4, 5, 6, 7, 8, 9, 10 (or more), optionally wherein the hydrophobic residues are independently selected at each hydrophobic position from L- or D-forms of 3,3-diphenyl-alanine, 3-(1-naphthyl) alanine, 3-(2-naphthyl) alanine, 2,6-dimethyl-tyrosine, 2-aminooctanoic acid, 3-cyclohexylalanine, or optionally only a sub-set thereof consisting of 3-(1-naphthyl) alanine and 2,6-dimethyl-tyrosine.

The use of at least one sequence from within SEQ ID NO:512 (or at least one sequence within any of SEQ ID NO:513 to SEQ ID NO:522, e.g. a sequence(s) selected from SEQ ID NO:631 to SEQ ID NO:638) as a Cell Penetrating Peptide (CPP) is componentry to this disclosure; for use to transport a cargo into a cell (by its conjugation to the cargo and administration to the cell); for use in a fusion protein to transport a cargo peptide/protein sequence into a cell; wherein any cargo, including any cargo peptide/protein sequence cargo, including any polynucleotide cargo, including any drug, including any FDA/EMA approved drug, including any anti-cancer/anti-viral drug (e.g. FDA/EMA approved), is contemplated. In some embodiments, transport into the mitochondrial matrix is contemplated.

Incidentally, the use of a non-peptide mitochondrial localizing moiety (non-limiting e.g. refer [222-224]) such as MitoQ (been used in clinical trials, or SKQ [or derivative thereof, refer work of Professor Vladimir P. Skulachev]) or its componentry triphenylphosphonium (TPP) moiety, or [225], is hereby contemplated, as is the use of any non-peptide cell penetrating moiety (e.g. SMOCs that can be easily attached to biomolecules {Okuyama et al (2007) Nature Methods Volume 4 p 153}, e.g. addition of guanidinium group(s) enhances cell penetration {Elson-Scwab et al (2007) J Biol Chem Volume 282 p 13585}).

### Cycle(s) Thereof

It will be appreciated that an amino acid sequence of/in this disclosure can be readily cyclated to form a cyclic/cyclated amino acid sequence of this disclosure. For non-liming example, cyclization of amino acid chains can be achieved by sidechain-to-sidechain, sidechain-to-backbone, or head-to-tail (C-terminus to N-terminus, which then confers the advantage that there is no C-terminus or N-terminus for exopeptidases to attack) cyclization techniques, which can, for non-limiting example, be synthesized using either classical solution-phase linear peptide cyclization or resin-based cyclization, or other method taught in U.S. Pat. No. 10,053,677B2 and/or U.S. Pat. No. 10,729,749B2 and references therein.

For a linear amino acid sequence of this disclosure/herein, corresponding cyclic forms, optionally wherein the cycle(s) is formed by use of a scaffold(s) (e.g. as in U.S. Pat. No. 10,624,968B2 and references therein), including multi-cyclic (e.g. bi-, tri-cyclic etc.) and/or cyclic forms with one or more of an “overhang/excess” sequence (which is not in a cycle) are componentry to this disclosure. Optionally, wherein a cycle is formed by the formation of a peptide bond between the N- and C-terminal residues of a linear amino acid sequence. Optionally, wherein a cycle(s) is created by judicious use of one or more cysteine residues naturally in the sequence, and/or one or more cysteine residues inserted/substituted into the sequence, optionally at an end(s), wherein a pair(s) of cysteine residues can bind, via their side-chains, by a disulphide bond. Optionally wherein at least two cysteine residues in the sequence are connected in parallel (in 3-D) by a CPP sequence (e.g. a poly-Arginine sequence, optionally containing one or more D-amino acids) with at least two cysteine residues in it, optionally at its N- and C-terminal ends. Bicyclic forms, with a CPP sequence in one cycle, and the cargo sequence (which can reduce/inhibit F₁F₀ ATP hydrolysis e.g. in a cell and/or a Sub-Mitochondrial Particle [SMP] assay of F₁F₀ ATP hydrolysis) in the other, are componentry to this disclosure, as is their use thereof (for at least one use disclosed herein), wherein it is well known in the art how to produce such bicyclic forms (with CPP in one cycle, and cargo sequence in other cycle) e.g. refer to U.S. Pat. No. 10,626,147B2, references therein, and patents or applications thereof that cite it.

### Scaffold Thereof

In some embodiments, a protein(s)/peptide(s) of/in this disclosure is combined with a molecular scaffold(s), some non-limiting examples of which are in U.S. Pat. No. 10,624,968B2, US2018/0280525A1, WO2016/067035A1, WO2017/191460A1 and references cited therein. In some embodiments, use thereof of a scaffold reduces susceptibility to one or more proteolytic enzyme species, which improves the plasma stability of the protein/peptide in the blood of a subject (that has a blood-based circulatory system). Preferably the scaffold is of known and low toxicity. Preferably the scaffold comprises reactive groups that are capable of reacting with functional group(s) of the protein(s)/peptide(s) to form covalent bonds. The scaffold may comprise one or more chemical group(s) which form the linkage with a protein(s)/peptide(s), such as (without limitation) amines, thiols, alcohols, ketones, aldehydes, nitriles, carboxylic acids, esters, alkenes, alkynes, azides, anhydrides, succinimides, maleimides, alkyl halides and acyl halides.

### Some Cyclic Peptide/Protein Embodiments of the Disclosure

Cyclic/bi-cyclic peptide/protein that comprises (or consists of) at least one (any) IF1 protein/fragment (or sequence variant thereof); (any) IF1 protein/fragment (or sequence variant thereof) within a cyclic/bi-cyclic peptide/protein;
Cyclic, instead of linear, form of SEQ ID NO:X, including
cyclic/bicyclic form comprising (or consisting of):
(SEQ ID NO:X)_{y}-Cys- (SEQ ID NO:X)ₘ-Cys- (SEQ ID NO:X)ᵥ; or
(SEQ ID NO:X)_{y}-Cys- (SEQ ID NO:X)ₙ-Cys- (SEQ ID NO:X)ₘ-Cys- (SEQ ID NO:X)ᵥ;
which, when y and v equal 0, is:
Cys- (SEQ ID NO:X)ₘ-Cys; or
Cys- (SEQ ID NO:X)ₙ-Cys- (SEQ ID NO:X)ₘ-Cys;
wherein y, n, m, v, are independently selected from an integer within the range 0-4, Cys is cysteine [or variant thereof] (preferably with its side-chain bound to a scaffold structure by a thioether or disulphide bond, wherein a number of scaffold options to combine to, and how to combine to each, is taught by one or more of [226-231], U.S. Pat. No. 10,624,968B2, US2018/0280525A1, WO2016/067035A1, WO2017/191460A1 and/or other paper/patent/patent application output of Bicycle Therapeutics [which has corporate entities in the UK [Ltd.] and USA [Inc.]], [232-234], U.S. Pat. No. 7,538,085B2, U.S. Pat. No. 10,626,147B2, U.S. Pat. No. 10,736,932B2, US20190284239A1, US2020/0291070A1, WO2019/148194A2, WO2019/148195A2, and/or other paper/patent/patent application output of Entrada Therapeutics Inc. and/or its founder Dehua Pei), X is independently selected for each occurrence of X from 1, or the number of sequences in the Sequence Listing component of this present application, or any integer between 1 and the total number of sequences in the Sequence Listing component of this present application, wherein SEQ ID NO:X in this context (and optionally other contexts herein also) can also refer to fragment of SEQ ID NO:X, or concatenated fragments of SEQ ID NO:X, and/or sequence variant of SEQ ID NO:X;
SEQ ID NO:X in each case thereof can be integrated into the sequence in either orientation (preferably N to C-terminal orientation);
in some embodiments of a bicycle structure, SEQ ID NO:X in one cycle is a Cell Penetrating Peptide (CPP) sequence, optionally selected from SEQ ID NO:124 to SEQ ID NO:126 or SEQ ID NO:440 to SEQ ID NO:638 (wherein some of SEQ ID NO:551 to SEQ ID NO:629 are shown suited to confer cellular entry of cyclic/bi-cyclic peptides in [220, 221, 232], U.S. Pat. No. 10,626,147B2, U.S. Pat. No. 10,501,496B2, US2019/0309020A1, US2017/0355730A1, US2017/0190743A1, US2019/0282654A1, US2019/0284240A1, WO2015/179691A2) and SEQ ID NO:X in the other cycle comprises (or consists of) at least one “immature” (with Mitochondrial Import Sequence, MIS) or “mature” (without MIS) IF1 protein/fragment (or sequence variant) thereof, or an MIS sequence concatenated to an IF1 protein fragment (or sequence variant thereof), optionally at least one sequence selected from SEQ ID NO:5 to SEQ ID NO:123, or a fragment (or concatenation of fragments) thereof, optionally-without its epitope/affinity tag and CPP components—at least one of SEQ ID NO:166 to SEQ ID NO:335 or SEQ ID NO:338 to SEQ ID NO:438;
in some embodiments, the sequence is bound to a scaffold as below, wherein each S atom, in each side-chain of shown Cys residues, is bound to a carbon of the scaffold by a thioether bond:

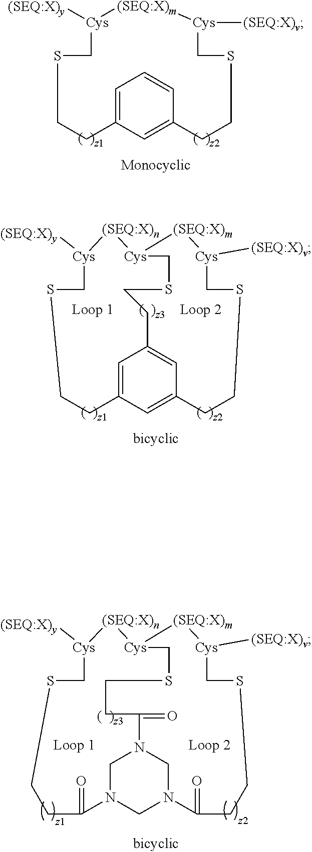

in some alternative embodiments, the sequence is bound to a scaffold as below, wherein in the structure on the left, (unlike above) the S atom in the Cys side-chain is not explicitly shown, and unlike above, the S atom in the Cys side chain binds the scaffold (an S atom of the scaffold) by a disulphide (S—S) bond (disulphide bonds denoted by dashed lines), wherein the second Cys isn't shown because a variant is in its place, which has CONH₂ instead of COOH (i.e. the C-terminal end is amidated, which simultaneously takes away a negative charge and makes this C-terminal end less susceptible to exopeptidases; cases where this end is COOH instead are also contemplated); note that disulphide bonds break within the reducing intracellular environment: it is preferred to have a CPP sequence in the left hand loop (left as one looks at the page) and in the right hand loop a “mature” IF1 protein/fragment (or sequence variant thereof), preferably with a Mitochondrial Import Sequence [MIS] N-terminal to it:

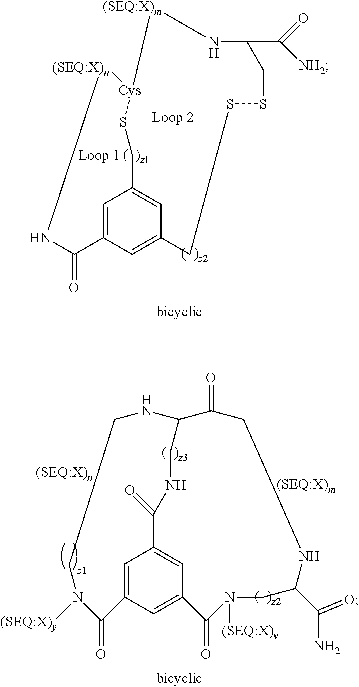

wherein SEQ: X=SEQ ID NO:X, z1, z2, z3 are independently selected from an integer between 0-100, wherein a sub-range of 1-50 is preferred in some embodiments, 1-10 in others, 1-5 in others, 0-7 in others;
X independently selected at each point of use, z1, z2, z3, y, n, m, v: all possible value (within their designated ranges) combinations thereof are contemplated across different embodiments;
note that in some embodiments (here and in other contexts herein), what SEQ ID NO:X can refer to isn't restricted to the sequences in the Sequence Listing component to this disclosure and can instead refer to any sequence of/in, e.g. taught by, this disclosure;
in some embodiments, at least one cycle of the bicyclic/cyclic structure comprises (or consists of) an IF1 protein fragment that contains the H49 (“mature” [without MIS] IF1 protein numbering) residue and up to 3 or 4 or 5 or 6 or 7 or 8 or 9 or 10 or 11 or 12 or 13 or 14 or 15 surrounding residues, or an IF1 protein fragment from a part of the IF1 protein that is more C-terminal than H49, optionally with a conjoined MIS sequence N-terminal to it.

A polypeptide comprising at least three cysteine (or variant thereof) residues, separated by at least two loop sequences, and a molecular scaffold which forms covalent bonds with the cysteine residues of the polypeptide such that at least two polypeptide loops are formed on the molecular scaffold, which can inhibit/reduce F₁F₀ ATP hydrolysis (e.g. in a cell and/or in a Sub-Mitochondrial Particle [SMP] assay of F₁F₀ ATP hydrolysis), and/or a pharmaceutically-acceptable salt, solvate, hydrate, prodrug, liposome, nanoparticle (e.g. lipid nanoparticle, LNP) or other vector of the art thereof.

Some Further Sequences of this Disclosure

All IF1 proteins (from all species, especially from eukaryotes, particularly from mammals), and sequence variant(s) thereof, and all IF1 protein fragments, and sequence variant(s) thereof, and concatenation(s) thereof, and any fusion protein incorporating at least one of the aforementioned, are componentry to this disclosure, as are the nucleotide sequences that code for them, as is the use of at least one of the aforementioned, for at least one use disclosed herein. Any IF1 protein/fragment (or sequence variant thereof), from any species, and use thereof (for at least one use disclosed herein). Any polynucleotide, gene (if applicable), DNA, cDNA, RNA, mRNA sequence that codes (via the genetic code [wherein its redundant character is well known] or a known variant thereof [e.g. mitochondrial]) for any IF1 protein/fragment (or sequence variant thereof), and use thereof (for at least one use disclosed herein), wherein the opposite, complimentary base-pairing strand to the coding sequence is also contemplated and any nucleotide sequence that can hybridize to it “under stringent conditions” (term well understood in the art, illustrative definition included herein), as is the peptide/protein sequence that it encodes, and its use thereof (for at least one use disclosed herein). Given that many species have one or more of an IF1 protein sequence, and there are many species, there are many IF1 protein embodiments of the present disclosure. Although IF1 protein sequences can vary between species, they are typically functionally interchangeable between different species [141], wherein even a yeast IF1 protein sequence can inhibit bovine F₁F₀ ATP hydrolysis [234]. One of the art will know how to find further IF1 protein sequences of other/same species, not shown here for brevity, but which are also componentry to this disclosure (as are their fragments, and concatenated fragments thereof), as are their sequence variants, for example, those with their “phosphorylation control switch” residue, and/or one or more of their “pH dependence motif” residues, changed to another amino acid (these element terms are explained by FIG. 10 and its legend). For example, to find further IF1 protein sequences of other/same species, one of the art can refer to InterPro family “Mitochondrial ATPase inhibitor (IPR007648)” and/or Pfam family “IATP (PF04568)”. All protein sequences in the IPR007648 and/or PF04568 protein families (and sequence variants thereof), and their sub-sequences/fragments (and sequence variants thereof), and their concatenated fragments (and sequence variants thereof), are componentry to this disclosure, and may be woven into any of the fusion proteins herein, substituting for any element therein. Or, for example, one of the art can use an IF1 protein sequence, optionally the human IF1 protein sequence (UniProtKB: Q9UII2, SV=1), as the query string in a “Basic Local Alignment Search Tool” (BLAST, https://blast.ncbi.nlm.nih.gov) search of a protein database (protein BLAST, BLASTp), optionally the “non-redundant protein sequences” (nr) database at the National Center for Biotechnology Information (NCBI) which is one of the database options offered with NCBI BLAST, and/or use BLAST at UniProt and search the UniProt protein database https://www.uniprot.org/blast), and from the results outputted select a protein sequence(s) of high BLAST score/percentage sequence identity/similarity (in different non-limiting embodiments: >99%, >98%, >95%, >90%, >80%, >70%, >60%, >50%, >40%, >30% sequence similarity) to the query protein (wherein BLAST results are typically/optionally returned to the user in order of BLAST score from high to low) and/or that have one or more IF1 protein motif characteristics, such as the “pH dependence motif”. Especially preferred are those proteins that have been annotated in the database as an IF1 protein and/or as a protein product of an ATP5IF1 (alternatively known as ATPIF1) gene, wherein the human ATP5IF1 gene has HUGO Gene Nomenclature Committee (HGNC) ID of HGNC: 871, and IF1 protein products of orthologs of this gene are componentry to the present disclosure. One of the art knows, and/or knows how to find, multiple online methods to find orthologs e.g. using OrthoDB (www.orthodb.org, to illustrate, refer to Group 1566610at2759 and/or Group 212481at40674 therein). All protein products of an ATP5IF1 gene are componentry to the present disclosure. For example, the human ATP5IF1 gene has three protein products: primary accession numbers in UniProtKB: Q9UII2, Q9UII2-2 and Q9UII2-3, which are all componentry to this disclosure. When a single species has multiple IF1 proteins/homologs, e.g. Heterocephalus glaber has G5AP86 and A0A0P6J910 (UniProtkb primary accession numbers), e.g. Mus musculus has 035143, E9PV44 and Q8BTA7, e.g. Oryctolagus cuniculus has G1SEZ3, G1TES2 and G1U0F8, they are all contemplated by, and componentry to, this disclosure.

FIG. 10 presents some exemplifications of SEQ ID NO:38, and some other peptide/protein inhibitors of F₁F₀ ATP hydrolysis, which are in turn SEQ ID NO:639 to SEQ ID NO:1425.

Protein sequences in InterPro “IPR007648” are incorporated herein in their entirety by reference, incorporated herein in SEQ ID NO:[B+Z], where B is the number of sequences in the Sequence Listing of this application and Z is any integer in the numerical range 1 to the number of protein sequences (optionally/preferably only including the latest sequence version for each entry/UniProtKB primary accession number) in InterPro “IPR007648”.

Peptide/protein comprising (or consisting of) at least one of SEQ ID NO:X, and/or at least one fragment of at least one of SEQ ID NO:X, and/or concatenated fragments of at least one of SEQ ID NO:X, and/or at least one functional sequence variant thereof, and its use thereof (for at least one use disclosed herein); componentry to the present disclosure, and its use thereof (for at least one use disclosed herein), is SEQ ID NO:X [wherein X can be 1, or the number of sequences in the Sequence Listing component to this application, or any integer between 1 and the total number of sequences in the Sequence Listing component to this application {or a higher number to include any sequences incorporated by reference}] and any fragment/sub-sequence thereof, and any concatenated fragments thereof, and any “conservatively modified variant” thereof (including any “conservatively modified variant” of fragment/concatenation of fragments thereof), and any functional sequence variant thereof (including any functional sequence variant of fragment/concatenation of fragments thereof), wherein “functional” in this context is an ability to inhibit/reduce F₁F₀ ATP hydrolysis, for example inside a cell or in a Sub-Mitochondrial Particle (SMP) assay of F₁F₀ ATP hydrolysis;
of a peptide/protein sequence of/in this disclosure, any functional sequence variant thereof, any functional fragment thereof (including any functional sequence variant thereof), any functional concatenation of fragments thereof (including any functional sequence variant thereof), is a sequence of this disclosure (and its use is contemplated, for at least one use disclosed herein);
At any point that SEQ ID NO:X is referred to herein, wherein X can be at least 1, or at least the number of sequences in the Sequence Listing component to this application, or at least any integer between 1 and the total number of sequences in the Sequence Listing component to this application, then in alternative embodiments SEQ ID NO:X, independently at each point of use, refers to a functional sequence variant of SEQ ID NO:X (or a functional sequence variant of a fragment, or a concatenation of fragments, thereof), wherein “functional” is as earlier defined.

Componentry to this disclosure is:
a nucleotide(s) and/or amino acid sequence(s) of/in this disclosure produced/isolated/purified/substantially purified/partially purified;
a produced/isolated/purified/substantially purified/partially purified polynucleotide comprising (or consisting of) at least one nucleotide sequence of/in this disclosure (or a fragment thereof, or concatenated fragments thereof);
a produced/isolated/purified/substantially purified/partially purified peptide/protein comprising (or consisting of) at least one amino acid sequence of/in this disclosure (or a fragment thereof, or concatenated fragments thereof);
a cell-free and serum-free composition (preferably lacking any other human protein) comprising (or consisting of) a peptide/protein comprising (or consisting of) an amino acid sequence of/in this disclosure;
a polynucleotide comprising (or consisting of) at least one nucleotide sequence of/in this disclosure (or a fragment thereof, or concatenated fragments thereof); a peptide/protein comprising (or consisting of) at least one amino acid sequence of/in this disclosure (or a fragment thereof, or concatenated fragments thereof);
In some embodiments, a peptide/protein of this disclosure is at least 95% pure, and/or at least 99% pure, and/or substantially free from impurity/impurities.

Any sub-sequence/fragment of an amino acid/nucleotide sequence of/in this disclosure, and/or use thereof (for at least one use disclosed herein), is componentry to this disclosure. Any concatenation of sub-sequences/fragments of at least one amino acid/nucleotide sequence of/in this disclosure, and/or use thereof (for at least one use disclosed herein), is componentry to this disclosure.

A (optionally produced/isolated/purified/substantially purified/partially purified) polynucleotide encoding at least one amino acid sequence of/in this disclosure. DNA comprising (or consisting of) an intronless sequence encoding at least one amino acid sequence of/in this disclosure. cDNA encoding at least one amino acid sequence of/in this disclosure. DNA/cDNA encoding at least one amino acid sequence of/in this disclosure or a degenerate variant thereof. DNA comprising (or consisting of) (a) cDNA encoding at least one amino acid sequence of/in this disclosure, or (b) a degenerate variant of the cDNA.

Every nucleotide sequence (gene [if applicable, preferably isolated/produced], DNA, RNA, mRNA, cDNA) that codes, by the genetic code, for a peptide/protein/amino acid sequence of/in this disclosure is also componentry to this disclosure (as is each opposite complimentary base pairing strand to the coding sequence, optionally wherein the coding and anti-coding strands exist as a double-stranded polynucleotide), as is its use thereof (for at least one use disclosed herein). Componentry to this disclosure is to optimize the codons used, dependent upon which species, and/or intracellular compartment (e.g. mitochondria or nucleus), the nucleotide sequence will (primarily) be expressed in. Wherein this optimization is to use the most commonly employed codon for each amino acid, in the applicable type of intracellular compartment, in the applicable species, wherein there is publically available data on codon usage in different species (illustrative, non-limiting e.g. refer to https://www.genscript.com/tools/codon-frequency-table, http://www.kazusa.or.jp/codon/, https://www.bioinformatics.org/sms2/rev_trans.html). Nucleotide sequences of/in this disclosure can be single- or double-stranded (or contain both single- and double-stranded portions, and/or contain triple-stranded portions). All these forms are contemplated and componentry to the disclosure, as is their use thereof (for at least one use disclosed herein).

In relation to an amino acid or nucleotide sequence of/in this disclosure, “conservatively modified variants thereof” (USPTO terminology) are also componentry to this disclosure, as is their use thereof (for at least one use disclosed herein). In relation to an amino acid sequence of/in this disclosure, I report that sequence variants exist, and a “VARIANT” thereof (WIPO ST.25 terminology) is also componentry to this disclosure, as is its use thereof (for at least one use disclosed herein).

A sequence variant(s) of an amino acid sequence of/in this disclosure, which can also inhibit/reduce F₁F₀ ATP hydrolysis (for example inside a cell, preferably a eukaryote cell, or in an SMP assay of F₁F₀ ATP hydrolysis), is also componentry to this disclosure, as is its use thereof (for at least one use disclosed herein), as are the nucleotide sequences (gene [if applicable, preferably isolated/produced], DNA, RNA, mRNA, cDNA) that code for it, as is the use of one or more these thereof (for at least one use disclosed herein).

Componentry to this disclosure is:
a peptide/protein that has, or (in other embodiments) which has a region that has, at least 30% (or, in alternative embodiments [wherein each different following term [>% value] is a different embodiment]: >35%, or >40%, or >45%, or >50%, or >55%, or >60%, or >65%, or >70%, or >75%, or >80%, or >85%, or >90%, or >95%, or >96%, or >97%, or >98%, or >99%, or >99.5%) sequence identity to an IF1 protein or a fragment thereof, or concatenated fragments thereof, which can inhibit/reduce F₁F₀ ATP hydrolysis (for example inside a cell, preferably a eukaryote cell, or in a Sub-Mitochondrial Particle [SMP] assay of F₁F₀ ATP hydrolysis), and its use thereof (for at least one use disclosed herein); DNA with at least 30% (or, in alternative embodiments [wherein each different following term [>% value] is a different embodiment]: >35%, or >40%, or >45%, or >50%, or >55%, or >60%, or >65%, or >70%, or >75%, or >80%, or >85%, or >90%, or >95%, or >96%, or >97%, or >98%, or >99%, or >99.5%) sequence identity to the cDNA of an IF1 protein or a fragment thereof, or concatenated fragments thereof, wherein its coded amino acid sequence can inhibit/reduce F₁F₀ ATP hydrolysis (for example inside a cell, preferably a eukaryote cell, or in an SMP assay of F₁F₀ ATP hydrolysis), and the use of this DNA and/or its coded for peptide/protein (for at least one use disclosed herein) is hereby contemplated;
RNA (e.g. mRNA) with at least 30% (or, in alternative embodiments [wherein each different following term [>% value] is a different embodiment]: >35%, or >40%, or >45%, or >50%, or >55%, or >60%, or >65%, or >70%, or >75%, or >80%, or >85%, or >90%, or >95%, or >96%, or >97%, or >98%, or >99%, or >99.5%) sequence identity to the mRNA of an IF1 protein or a fragment thereof, or concatenated fragments thereof, wherein its coded amino acid sequence can inhibit/reduce F₁F₀ ATP hydrolysis (for example inside a cell, preferably a eukaryote cell, or in an SMP assay of F₁F₀ ATP hydrolysis), and the use of this RNA and/or its coded for peptide/protein (for at least one use disclosed herein) is hereby contemplated.

Any peptide/protein/amino acid sequence that has notable sequence similarity/identity/homology (at least >30%, and in different embodiments: >40%, >50%, >60%, >65%, >70%, >75%, >80%, >85%, >90%, >95%, >98%, >99%, >99.5%, >99.8% sequence similarity/identity/homology) to a peptide/protein/amino acid sequence (and/or to a fragment thereof, and/or concatenated fragments thereof) of/in this disclosure is also componentry to this disclosure, as is its use thereof (for at least one use disclosed herein), as are the nucleotide sequences (gene [if applicable, preferably isolated/produced], DNA, RNA, mRNA, cDNA) that code for it, as is the use of one or more these thereof (for at least one use disclosed herein). Any peptide/protein/amino acid sequence that has notable sequence similarity/identity/homology (at least >30%, and in different embodiments: >40%, >50%, >60%, >70%, >80%, >90%, >95%, >98%, >99%, >99.5%, >99.8% sequence similarity/identity/homology) to a peptide/protein/amino acid sequence (and/or to a fragment thereof, and/or concatenated fragments thereof) of/in this disclosure, which can also inhibit/reduce F₁F₀ ATP hydrolysis (for example inside a cell, preferably a eukaryote cell, or in a Sub-Mitochondrial Particle [SMP] assay of F₁F₀ ATP hydrolysis), is also componentry to this disclosure, as is its use thereof (for at least one use disclosed herein), as are the nucleotide sequences (gene [if applicable, preferably isolated/produced], DNA, RNA, mRNA, cDNA) that code for it, as is the use of one or more these thereof (for at least one use disclosed herein).

Any nucleotide sequence that has notable sequence similarity/identity/homology (at least >30%, and in different embodiments: >40%, >50%, >60%, >65%, >70%, >75%, >80%, >85%, >90%, >95%, >98%, >99%, >99.5%, >99.8% sequence similarity/identity/homology) to a nucleotide sequence (and/or to a fragment thereof, and/or concatenated fragments thereof) of/in this disclosure is also componentry to this disclosure, as is its use thereof (for at least one use disclosed herein), as is the amino acid sequence (peptide/protein) that it codes for, as is its use thereof (for at least one use disclosed herein). Any nucleotide sequence that has notable sequence similarity/identity/homology (at least >30%, and in different embodiments: >40%, >50%, >60%, >65%, >70%, >75%, >80%, >85%, >90%, >95%, >98%, >99%, >99.5%, >99.8% sequence similarity/identity/homology) to a nucleotide sequence (and/or to a fragment thereof, and/or concatenated fragments thereof) of/in this disclosure is also componentry to this disclosure, as is its use thereof (for at least one use disclosed herein), wherein it too encodes an amino acid sequence (peptide/protein) that can inhibit/reduce F₁F₀ ATP hydrolysis (for example inside a cell, preferably a eukaryote cell, or in a Sub-Mitochondrial Particle [SMP] assay of F₁F₀ ATP hydrolysis), wherein the use (for at least one use disclosed herein) of this amino acid sequence (peptide/protein) is also componentry to this disclosure.

Conservatively modified peptide/protein sequence variants of a sequence of/in this disclosure are themselves sequences of this disclosure, especially those that retain biological activity, wherein they can still inhibit/reduce F₁F₀ ATP hydrolysis (e.g. in a cell and/or in a Sub-Mitochondrial Particle [SMP] assay of F₁F₀ ATP hydrolysis), and their use (for at least one use disclosed herein) is herein contemplated. One of the art knows, optionally guided by software (e.g. DNASTAR Software, optionally DNASTAR Lasergene package [DNASTAR, Masidon Wisconsin, USA]), amino acid substitution(s) that highly conserve one or more structural/chemical properties such as one or more of polarity, charge, solubility, hydrophobicity, hydrophilicity, amphipathic character and so can select amino acid sequence substitution(s), optionally with one or more amino acid insertions/deletions, that retain biological activity, which can still inhibit/reduce F₁F₀ ATP hydrolysis. Conservative amino acid substitutions may include synonymous amino acid residues within a group which have sufficiently similar physicochemical properties, so that a substitution between members of the group will preserve the biological activity of the molecule (see e.g. Grantham, R. (1974), Science 185, 862-864). Preferably, synonymous amino acid residues, which are classified into the same groups and are typically exchangeable by conservative amino acid substitutions, are defined in Table 2 of U.S. Pat. No. 8,080,517B2 (herein incorporated by reference). Other groupings of amino acids by physicochemical properties (and/or similar side-chains) are known to those of the art and incorporated herein e.g. refer [P14], U.S. Pat. No. 9,255,124B2, and can include non-proteinogenic amino acids e.g. refer U.S. Pat. No. 10,626,147B2. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic/positive side chains is lysine, arginine and histidine, a group of amino acids having negative side chains is aspartic acid and glutamic acid, small non-polar, hydrophobic amino acids include glycine, alanine, leucine, isoleucine, valine, proline, and methionine, large non-polar, hydrophobic amino acids include phenylalanine, tryptophan and tyrosine, polar neutral amino acids include serine, threonine, cysteine, asparagine and glutamine, and a group of amino acids having sulfur-containing side chain is cysteine and methionine. Some (non-limiting) preferred conservative amino acid substitution groups are: valine-leucine-isoleucine; phenylalanine-tyrosine; lysine-arginine; alanine-valine; glutamic-aspartic; and asparagine-glutamine. Some tools of the art to identify conservative substitutions include Grantham's distance, Sneath's index, Epstein's coefficient of difference, Miyata's distance, Experimental Exchangeability (e.g. see [235], or a similar study).

To determine the percent identity of two amino acid sequences, or of two nucleotide sequences, the sequences are aligned by one of the art, and/or an algorithm of the art, for optimal comparison purposes (e.g. in some embodiments, gap(s) can be introduced in the first sequence, and/or in the second sequence, for optimal alignment of the first with the second sequence). After this alignment, the amino acid/nucleotide residues at corresponding amino acid/nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences {i.e. % identity=(number of identical overlapping positions/total number of positions)*100}. Optionally, the determination of percent identity between two sequences is accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set (e.g. for score=100, wordlength=12) to obtain nucleotide sequences similar/homologous to a nucleotide sequence of the present disclosure. BLAST protein searches can be performed with the XBLAST program parameters set (e.g. to Score=50, wordlength=3) to obtain amino acid sequences similar/homologous to a protein sequence of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST and/or PSI-Blast programs, the default parameters of the respective programs (e.g., of XBLAST and NBLAST) can be used (see e.g. the NCBI website, ncbi.nlm.nih.gov, where the BLAST family of programs can be found). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used. Alternative methods in the art (and/or programs) of calculating percent identity are also hereby contemplated. For example, the percent identity between two sequences can be determined using a technique(s)/algorithm(s) similar to one listed herein, with or without allowing gaps. Comparing the similarity/identity of sequences across the entirety of their length, or only part(s) thereof, are both contemplated (full/global or partial/local alignment). Another program of the art is FASTA (Pearson (1990), Methods Enzymol. 183, 63-98: Pearson and Lipman (1988), Proc. Natl. Acad. Sci. U.S.A 85, 2444-2448.) and others are the programs available in the Wisconsin Sequence Analysis Package, version 9.1 (Devereux et al., 1984, Nucleic Acids Res. 12, 387-395.), for example the programs BEST FIT and GAP may be used to determine the % identity between two polynucleotides and the % identity and the % homology between two polypeptide sequences. BESTFIT uses the “local homology’ algorithm of (Smith and Waterman (1981), J. Mol. Biol. 147, 195-197) and finds the best single region of similarity between two sequences.

A nucleotide sequence that can hybridize “under stringent conditions” with a nucleotide sequence of/in this disclosure is also componentry to this disclosure, as is its complementary (by base pairing rules) nucleotide sequence, and its use thereof (for at least one use disclosed herein), as well as the amino acid sequence(s) (peptide[s]/protein[s]) that it codes for, and its use thereof (for at least one use disclosed herein). A nucleotide sequence that can hybridize “under stringent conditions” with the complimentary base-pairing strand of a nucleotide sequence of/in this disclosure is also componentry to this disclosure, as is its use thereof (for at least one use disclosed herein), as well as the amino acid sequence(s) (peptide[s]/protein[s]) that it codes for, and its use thereof (for at least one use disclosed herein). A nucleotide sequence that can hybridize “under stringent conditions” with the complimentary base-pairing strand of a nucleotide sequence of/in this disclosure is also componentry to this disclosure, as is its use thereof (for at least one use disclosed herein), wherein it too encodes an amino acid sequence (peptide/protein) that can inhibit/reduce F₁F₀ ATP hydrolysis (for example inside a cell, preferably a eukaryote cell, or in a Sub-Mitochondrial Particle [SMP] assay of F₁F₀ ATP hydrolysis), wherein the use (for at least one use disclosed herein) of this amino acid sequence (peptide/protein) is also componentry to this disclosure. Herein “hybridizes under stringent conditions” describes conditions for hybridization and washing under which nucleotide sequences with notable complementary sequence identity, at least 30%, typically remain hybridized to each other. Such stringent conditions are known to those skilled in the art and some can, for example, be found in Current Protocols in Molecular Biology, John Wiley & Sons, NY. In a non-limiting example, stringent hybridization conditions are hybridization at 6*Sodium chloride/sodium citrate (SSC) at about 45° C., followed by one or more washes in 0.1*SSC, 0.2% SDS at about 68° C. In a more preferred non-limiting example, stringent hybridization conditions are hybridization in 6×SSC at about 45° C., followed by one or more washes in 0.2*SSC, 0.1% SDS at 50-65° C. (i.e. one or more washes at 50° C., 55° C., 60° C. or 65° C.). In other embodiments, the term “under stringent conditions” is redefined to refer to more stringent conditions (e.g. by using higher temperature) wherein only nucleotide sequences, in different embodiments, at least 40%, or at least 50%, or at least 60%, or at least 70%, or at least 75%, or at least 80%, or at least 85%, or at least 90%, or at least 95%, or at least 98%, or at least 99%, or at least 99.5%, complementary to each other typically remain hybridized to each other. In other different embodiments of this disclosure, the term “under stringent conditions” herein is redefined to refer to “stringent conditions” or “high stringency conditions” or “moderate stringency conditions” or “low stringency conditions” in U.S. Pat. No. 8,080,517B2 (herein incorporated in its entirety by reference).

Unless otherwise indicated, amino acid sequences herein are presented in the order from the “N” (or amino) terminus to the “C” (or carboxyl) terminus. Herein homo-polymer sequences can be represented by a one-letter amino acid symbol followed by the number of consecutive occurrences of that amino acid in the sequence (e.g. R7 represents a heptamer that consists of L-arginine residues). Generally in the art, the terms peptide, polypeptide, protein are amino acid sequences of different lengths, but wherein the cut-offs can be somewhat ambiguous even to those of the art, and so, to simplify, these terms are used completely interchangeably herein, to refer to any amino acid sequence, of any length. Although, herein, “peptide” does tend to be used for shorter, and “protein” for longer, amino acid sequences. Peptidomimetics of peptides of/in this disclosure are also componentry to this disclosure. Methods for preparing peptidomimetic compounds are well known in the art e.g. refer “Quantitative Drug Design”, CA Ramsden Gd., Chapter 17.2, F. Choplin Pergamon Press (1992).

For amino acid/nucleotide sequences of/in this disclosure, their “sequence variants”, “variants”, “mutants”, “derivatives”, “analogues”/“analogs” and “fragments” (and sequence variants, variants, mutants, derivatives, analogues/analogs thereof) thereof are also componentry to this disclosure (as is their use, for at least one use disclosed herein), wherein the width/parameters of the meaning of these terms is clear to one of the art, e.g. from the patent literature in this area (therapeutic proteins/peptides), wherein those preferred retain the ability to cause a reduction in F₁F₀ ATP hydrolysis (e.g. in a cell and/or a Sub-Mitochondrial Particle [SMP] assay of F₁F₀ ATP hydrolysis). Herein, when a sequence of/in this disclosure is referred to, simultaneously, in alternative embodiments: sequence variants, variants, mutants, derivatives, analogues/analogs and fragments (and sequence variants, variants, mutants, derivatives, analogues/analogs thereof) thereof, which retain the desired activity, are also referred to. Also contemplated is an amino acid/nucleotide sequence(s) of/in this disclosure as part of, within, a longer sequence (and its use thereof, for at least one use disclosed herein) e.g. a fusion protein thereof. Also contemplated are hybrids of one or more different sequences of/in this disclosure. Also contemplated is the combined use (for at least one use disclosed herein) of one or more sequences of/in this disclosure, whereby an additive/synergistic effect(s) is contemplated.

For an amino acid sequence of/in this disclosure, all possible stereoisomeric forms of it (contemplating all chiral centres, including any in side-chains) are componentry to this disclosure e.g. L-amino acid or D-amino acid (or mixture thereof) are independently selected at each residue position, wherein isomeric, racemic, scalemate forms etc. are contemplated.

For a sequence of L-amino acids, its retroinverse/retro-inverse sequence is this sequence in reverse, wherein all the amino acids are D-amino acids. So, it is a sequence reversed with chirality inverted. In some cases, the amino acid side-chains of a sequence, and its retroinverse sequence, are equivalently arranged in relation to one another, wherein the retroinverse sequence is less susceptible to proteolysis by proteases. See e.g. Jameson et al., Nature, 368, 744-746(1994); Brady et al., Nature, 368, 692-693 (1994); Guichard et al, J. Med. Chem. 39, 2030-2039(1996), U.S. Pat. No. 8,080,517B2, US2018/0015137A1, U.S. Pat. No. 6,730,293B1. Retroinverse sequences of sequences of/in this disclosure are also sequences of this disclosure, as is their use (e.g. for at least one use disclosed herein). Mixed sequences, wherein only part of a sequence is its retroinverse, are also contemplated, as is their use (e.g. for at least one use disclosed herein). Retroinverse (part or entirety) sequences, wherein one or more of the amino acid residues with a chiral centre in their side-chain (isoleucine and threonine) are replaced with a different amino acid that doesn't (e.g. valine), are also componentry to this disclosure, as is their use (e.g. for at least one use disclosed herein). In a true retroinverse (part or entirety) sequence of/in this disclosure, L-isoleucine (S,S) is substituted for D-isoleucine (R,R) {but its substitution at zero or more places by D-allo-isoleucine (R,S) is also contemplated}, L-threonine (S,R) is substituted for D-threonine (R,S) {but its substitution at zero or more places by D-allo-threonine (R,R) is also contemplated}.

Componentry to this disclosure is any peptide/protein/amino acid sequence of/in this disclosure, wherein one or more of the following features applies (and when a peptide/protein/amino acid sequence is presented/referred to herein, in alternative embodiments, this referral is to the sequence modified such that one or more of the following features apply to it) [wherein all possible combinations below are contemplated {including all possible combinations of elements/descriptors within, and across, different bullet points} except those that are mutually exclusive]:
(1) Esterification of one or more carboxyl groups (e.g. at the C-terminal end and/or upon the side-chain of one or more aspartic acid (D) and/or glutamic acid (E) residues), e.g. as described in (with a moiety to attach selected from the options therein) one or more of (all incorporated herein, in their entirety, by reference) U.S. Pat. No. 9,790,483B2, U.S. Pat. No. 10,258,695B2, U.S. Pat. No. 10,428,323B2, U.S. Pat. No. 10,577,303B1, US2020/0032238A1, [193-194] and papers/patents/patent applications that cite one or more of these, preferably wherein the attached moiety, attached by an esterase labile ester bond, is hydrophobic (preferably neutral or positively charged), optionally wherein the attached moiety is (wherein it is shown below attached):

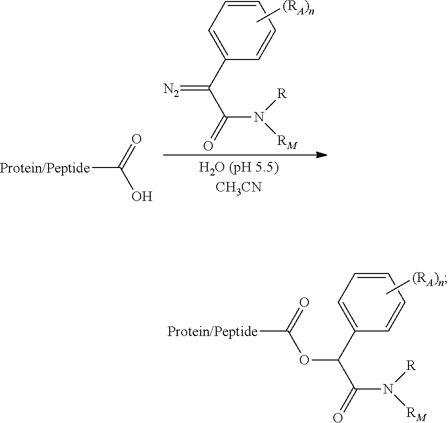

wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example:

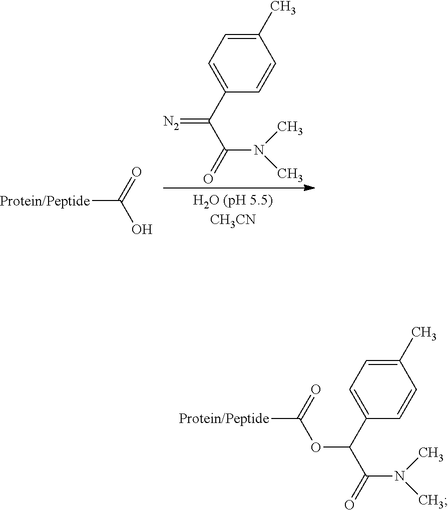

Optionally, wherein this esterification confers/assists cellular penetration, and/or renders less susceptibility to protease(s) action.
(2) Corresponding homo-amino acid, beta-homo-amino acid, N-methyl amino acid, alpha-methyl amino acid at one or more places in the sequence.
(3) D-amino acid in place of the corresponding L-amino acid, at one or more places. Replacement of one or more L-amino acid residues with one or more D-amino acid residues.
(4) D-amino acid(s) inserted at one or more places.
(5) In some embodiments, the peptide/protein comprises L amino acids. In some embodiments, the peptide/protein comprises D amino acids. In some embodiments, the peptide/protein comprises a mixture of D and L amino acids.
(6) L-isoleucine (S,S) substituted for, independently selected at each place of its substitution, D-isoleucine (R,R), or D-allo-isoleucine (R,S), or L-allo-isoleucine (S,R).
(7) L-threonine (S,R) substituted for, independently selected at each place of its substitution, D-threonine (R,S), or D-allo-threonine (R,R), or L-allo-threonine (S,S).
(8) Replacement of one or more amino acid residues with one or more non-natural/non-proteinogenic/non-eukaryote amino acid residues (incidentally, pyrrolysine is an example of a non-eukaryote amino acid). Insertion/substitution at one or more positions with a non-classical amino acid (e.g. an amino acid not ordinarily used by humans/mammals/eukaryotes/living systems). Some examples can be found in U.S. Pat. No. 10,626,147B2. If selecting to do this, preferred is to substitute a natural amino acid with a congener of that amino acid, wherein this is done at one or more places in the sequence, and/or one or more congeners of natural amino acids are inserted into the sequence. In some embodiments, one or more natural amino acids are replaced with a correspondingly, independently selected in each case, isosteric and/or isoelectronic non-natural amino acid. Preferred replacement(s) aren't recognised by degrading protease(s) and/or decrease the (potential) susceptibility of the peptide/protein to enzymatic degradation in the blood/body of a subject e.g. by removing a (potential) proteolytic attack/recognition site(s), most preferably wherein they increase the activity of the peptide/protein against its desired target(s) because of increased peptide/protein stability in the subject (so any reduced affinity to the peptide/protein's desired target(s), if applicable, is outweighed by more peptide/protein available for hitting the desired target(s), such that the total drug action, per unit drug, is increased, most preferably wherein the replacement(s) also actually increases the peptide/protein's affinity for the desired target(s)). Not only is replacement/insertion/deletion of an amino acid(s) in a suspected/proven protease recognition site(s) contemplated, but also near it (in the 2-D sequence and/or 3-D space), optionally wherein the addition/substitution of an amino acid(s) nearby blocks/restricts/reduces protease enzyme(s) access to its recognition site(s), optionally wherein a non-natural amino acid(s) may be used, optionally wherein at least one has a constrained/bulky amino acid side chain (e.g. [non-limiting] proline analogue(s), cyclo amino acid(s) {e.g. amino-cyclopropylcarboxylic acid}, Ca-disubstituted derivative(s) {e.g. aminoisobutyric acid}), such that proteolytic hydrolysis of the nearby peptide bond is conformationally/sterically impeded.
(9) Some non-limiting examples of non-natural amino acids to incorporate by substitution and/or insertion is any non-natural amino acid mentioned elsewhere herein and 1-naphthylalanine, 2-naphthylalanine, cyclohexylglycine, phenylglycine, tert-butylglycine, 3,4-dichlorophenylalanine, 4-fluorophenylalanine, homophenylalanine, 4-bromophenylalanine, pentafluorophenylalanine, N-methylphenylalanine, 4-chlorophenylalanine, cyclohexylalanine, ornithine, 2-amino butyric acid, N-methylglycine, N-methylalanine, N-methylleucine, norleucine, 3-amino-5-phenylpentanoic acid, 2,3-diaminopropionic acid, 2-amino butyric acid, citrulline, sarcosine, desmosine, norvaline, 4-aminobutyric acid, 2-aminobutyric acid, 6-aminohexanoic acid, 2-aminobenzoic acid, 4-aminobenzoic acid, 2,3-diaminopropionic acid, 2,4-diaminobutyric acid, cycloserine, carnitine, cystine, penicillamine, pyroglutamic acid, thienylalanine, hydroxyproline, allo-isoleucine, allo-threonine, isonipecotic acid, isoserine, statine, B-alanine, N-methyl amino acids, B-amino acids or Y-amino acids among others, as well as their derivates. A non-limiting list of unnatural amino acids can be found in “Unusual amino acids in peptide synthesis” by DC Roberts and F Vellaccio, in The Peptides, Gross E. and Meienhofer J. Eds. Academic Press, NY, USA and/or in the commercial catalogues of the companies specialized in the field, such as PolyPeptide Laboratories, Bachem, Novabiochem, Sigma-Aldrich, Peptides International, Advanced ChemTech, Chem-Impex, Maybridge Chemical, Chirotech Technology, Peninsula Laboratories or RSP Amino Acid Analogues among others.
(10) Replacement of one or more amino acid residues with one or more amino acid mimetics.
(11) Replacement of one or more amino acid residues with, and/or insertion of, one or more non-natural acids with a side-chain comprising a long (linear or branched, between 2-100 carbon atoms, preferably >10 and <20) aliphatic chain.
(12) Selenomethionine and/or N-formylmethionine in place of methionine at one or more places, hydroxyproline in place of proline at one or more places.
(13) Replacement of one or more amino acid residues with an alanine residue(s), e.g. as part of an “alanine scan”, e.g. to modify a (potential or proven) protease(s) recognition site(s).
(14) Replacement of one or more oxidation sensitive amino acid residues with one or more oxidation resistant amino acid residues.
(15) D-amino acid(s), and/or other non-proteinogenic amino acid(s), substituted/inserted at the N- and/or C-terminal ends to decrease susceptibility to exopeptidases. L-amino acid at N- and/or C-terminal end substituted for corresponding D-amino acid (illustrative e.g. L-arginine substituted for D-arginine).
(16) One or more D-amino acids, and/or other non-proteinogenic amino acid(s), are inserted/substituted into the unmodified sequence, and/or the amino acid sequence is changed (“conservative” substitution(s) preferred e.g. refer U.S. Pat. No. 9,255,124B2), and/or one or more amino acids are modified (e.g. with an amino acid modification(s) mentioned herein), at a site(s) in the sequence that is predicted to be susceptible to attack by an endopeptidase(s)/protease(s) (that is present in one or more of the species that will be administered, e.g. as predicted by https:/web.expasy.org/peptide_cutter/; so eliminating any relevant endopeptidase(s) recognition site(s) from the amino acid sequence) or that is actually observed to be a point of proteolysis by analysing fragments of the unmodified sequence after incubation with biological matter (from a species to be administered) for a specified period of time e.g. serum (optionally wherein after modification(s), this experiment is repeated and if an undesirable level of proteolysis is still occurring, the fragment species are again analysed, and a different modification(s) made accordingly). This method is componentry to this disclosure.
(17) Replacement of a negatively charged amino acid with a non-negatively charged amino acid at one or more positions.
(18) “Membrane permeant derivative” refers to a chemical derivative of a compound with increased membrane permeability of the compound, optionally because one or more hydrophilic groups are masked, optionally wherein these masking groups are cleaved off once inside the cell e.g. refer U.S. Pat. No. 5,741,657 for some non-limiting guidance.
(19) N-terminal and/or C-terminal modification(s). Including N- and/or C-terminus capping (introduction of a blocking group to the terminus via a covalent modification) to hamper endopeptidase activity, preferably wherein the added group(s) doesn't hamper the desired activity of the peptide/protein, preferably wherein the added group(s) increases lipophibicity of the peptide/protein. For non-limiting example, amidation/esterification of the C-terminus and/or acylation (e.g. acetylation) of the N-terminus. “Capping” with a different acyl group, more hydrophobic than acetyl, is herein contemplated. For non-limiting example, in increasing order of hydrophobicity (from less to more): formyl, acetyl, propanoyl, hexanoyl, myristoyl, palmitoyl, stearoyl. “Capping” the N-terminus with a benzyl group is contemplated, or a biotin group, or a protective group selected from a collection consisting of benzyloxycarbonyl (Z), terbutyloxycarbonyl (tPoc), fluorenylmethyloxycarbonyl (Fmoc), and allyloxycarbonyl (Alloc) groups. In some embodiments, a modified derivative comprises an N-terminal modification using suitable amino reactive chemistry, and/or C-terminal modification using suitable carboxy-reactive chemistry.
(20) Conjugation to one or more molecules/amino acid sequences that bind albumin (albumin-binding molecules).
(21) One or more fatty acids (or derivatives thereof) conjugated to the peptide/protein (preferably just one thereof conjugated). For non-limiting example, fatty acid attached to the N-terminus as an acyl group (illustrative e.g. myristoyl or palmitoyl or stearoyl group attached to N-terminus), and/or fatty acid bound to at least one amino acid side-chain (non-limiting e.g. to lysine side-chain, conjugated directly, or indirectly via a “spacer” moiety between), wherein the fatty acid can comprise/contain from 2 to 100 carbons, preferably >10 and <25 carbons, be saturated or unsaturated, linear or branched, substituted or non-substituted (e.g. hydroxylated or non-hydroxylated, e.g. sulfurated [e.g. SOH, SH or S-S] or non-sulfurated), cyclic or non-cyclic. A conjugated fatty acid(s) to a peptide/protein can confer self-association and/or its binding to albumin in the blood, which (sterically) decreases protease(s) access to it, and/or slows its renal clearance, thence increasing its half-life in blood (e.g. from minutes to hours). Conjugation of a fatty acid(s) reported/predicted to bind albumin is especially preferred, and/or conjugation of a fatty acid (and optionally its “spacer” moiety if applicable) present in a licensed/clinical candidate peptide/protein based drug, e.g. in one or more of insulin detemir, insulin degludec, liraglutide, semaglutide. Conjugation of a fatty acid(s), e.g. conjugation with palmitic acid (palmitoylation), can increase skin penetration of a peptide/protein. Contemplated is the binding of a fatty acid (or derivative thereof) to an already present or introduced/substituted in cysteine in the amino acid sequence, optionally via a disulphide bond, optionally wherein the fatty acid derivative chain is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated (or the fatty acid derivative is one found in one or more of [196], U.S. Pat. Nos. 5,907,030, 6,093,692, U.S. Pat. No. 6,225,445B1, U.S. Pat. No. 7,052,704B2, US2013/0053433A1, WO96/22773).
(22) Attachment (e.g. covalent) of one or more fatty acid molecules (optionally which can confer {optionally via conferring self-binding} binding to albumin in the blood, conferring less susceptibility to proteases and longer half-life in the blood [incidentally, as per the strategy used with Liraglutide]).
(23) An N-terminal and/or C-terminal modification shown in U.S. Pat. No. 8,946,166B2 (and/or in one or more of U.S. Pat. No. 9,067,967B2, U.S. Pat. No. 9,315,564B2, US2013/0078295A1, US2014/0322307A1, WO2014/170347A1, U.S. Pat. No. 6,372,717B1, U.S. Pat. No. 6,620,419B1, U.S. Pat. No. 6,974,799B2, U.S. Pat. No. 7,182,963B2, U.S. Pat. No. 7,998,493B2, U.S. Pat. No. 8,404,648B2, U.S. Pat. No. 10,660,839B2, U.S. Pat. No. 10,668,000B2, U.S. Pat. No. 10,668,000B2, US2004/0132667A1, US2018/0000717A1, WO2019149450A1, U.S. Pat. No. 7,863,417B2, U.S. Pat. No. 7,671,009B2).
(24) One or more cysteine residues, with a cholesterol derivative (e.g. cholesterol modified with a cysteine-reactive 2-bromoacetyl moiety) attached to its side-chain, are inserted/substituted into the amino acid sequence, optionally in place of unmodified cysteine(s).
(25) Modification at the C-terminus to remove its negative charge (and, optionally, simultaneously to increase lipophibicity e.g. a lipophilic moiety [e.g. cholesteryl/cholesterol moiety] attached to the C-terminus by an ester bond), for example by amidation, or by esterification, optionally wherein at the C-terminus, instead of COOH, there is C(O)R or C(O)N(H)R or C(O)NR₂ or R, and/or at the N terminus instead of NH₂ there is N(H)R or NR₂ or H or D or R or CH₃ or C(H₂)R or C(H)R₂ or CR₃, wherein R is independently at each point of use selected from alkyl (e.g. C(CH₃)₃), substituted alkyl (non-limiting examples: CF₃, CCl₃), deuterated alkyl (non-limiting example: CD₃), aminoalkyl, thioalkyl, alkoxy, halogen, haloalkyl, haloalkoxy, any atom or isotope permitted by valence (including any accompanying hydrogen(s)/deuterium(s) by valence e.g. (non-limiting), H, NH₂, SH, SiH₃, PH₂, BH₂ etc.) including, without limitation, La, Ti, Ce, V, Ta, Cr, Mo, Mn, Fe, Ru, Os, Co, Pd, Pt, Cu, Ag, Au, Zn, B, Al, Ga, C, Si, N, P, As, Sb, Bi, O, S, Se, F, Cl, Br, I, Hg.
(26) Modification(s) of one or more amino acids of an amino acid sequence of/in this disclosure, wherein the modification(s) is known to/predicted by those of the art to increase the plasma stability/half-life of a peptide/protein (e.g. by reducing susceptibility to cleavage by protease(s)), for example, wherein this has been shown with one or more peptide/protein examples in the literature.
(27) Modification(s) of one or more amino acids of an amino acid sequence of/in this disclosure, wherein the modification(s) is known to/predicted by those of the art to increase the lipophibicity/cellular penetration/ability to cross a biological/plasma membrane of a peptide/protein.
(28) Peptide/protein is N^{α}-alkylated (e.g. N^{α}-methylated) at one or more places. N-alkylation of one or more amide bonds within the peptide/protein.
(29) Substitution of the hydrogen on the alpha-carbon of one or more amino acid residues with another, independently selected in each case, chemical group.
(30) Peptide backbone length modification.
(31) Replacement of one or more peptide bonds with a surrogate bond.
(32) Non-peptide backbones or non-peptide backbone regions, including a peptoid backbone (e.g. as discussed in U.S. Pat. No. 6,730,293B1, as used in a CPP in and U.S. Pat. No. 6,730,293B1 [plus refer references therein], wherein use of any CPP in that paper/patent is hereby contemplated for the present disclosure), are hereby contemplated. In a peptoid backbone, sidechains are attached to the backbone nitrogen atoms rather than the backbone carbon atoms. Peptoid derivatives of the peptide/proteins of this disclosure may be readily designed from knowledge of the amino acid sequence. Retropeptoids (in which all amino acids are replaced by peptoid residues in reversed order) are also suitable derivatives in accordance with this disclosure. Peptide and/or peptoid and/or retropeptoid hybrids are contemplated.
(33) One or more post-translational modifications.
(34) Modification (e.g. a post-translational modification(s)) of one or more amino acid residues. Illustrative modifications include, without restriction, N-terminus modification(s), C-terminus modification(s), backbone modification(s), peptide bond modification(s) (e.g. —CH₂—NH—, —CH₂—S—, —CH₂S═O, O═C—NH, —CH₂—O—, —CH₂—CH₂—, S—C—NH—, —CH═CH—, —CF═CH—), removing the initiator methionine residue, adding an initiator methionine residue, attachment of one or more blocking/protecting groups (e.g. N- and/or C-terminal blocking group), derivatization, proteolytic cleavage(s), linkage to a cellular ligand(s) and/or other protein(s)/peptide(s)/amino acid sequence(s), isomerization, methylation (e.g. of one or more lysine and/or arginine residues), alkylation, halogenation (e.g. chlorination), phosphorylation (e.g. of one or more serine, threonine, tyrosine, aspartic acid, histidine residues), glycation, glycosylation, O-linked glycosylation, N-linked glycosylation, C-linked glycosylation, attachment of one or more of a sugar/carbohydrate/glycan (mono- or polysaccharide) at one or more places (e.g. on an amino-acid side-chain(s) and/or at one or more points on the peptide/protein backbone), lipidation, acylation (N-terminal or other), acetylation (N-terminal or other), amidation (for example upon the C-terminus), deamidation, formylation (e.g. of N-terminal methionine), sulfation, sulfatation (e.g. of one or more tyrosine residues), succinylation, butyrylation, carbamylation, carbonylation, oxidation, biotinylation, PEGylation, polyethylene glycol (PEG) chain(s) are attached covalently, cyclization, backbone modification, gamma-carboxylation, gamma-carboxyglutamic acid hydroxylation (e.g. of one or more of asparagine, aspartic acid, proline, lysine residues), pyrrolidone carboxylic acid (e.g. N-terminal glutamate that has formed an internal cyclic lactam), polysialylation, malonylation, hydroxylation, iodination, nucleotide addition (e.g. ADP-ribosylation), phosphate ester (O-linked) or phosphoramidate (N-linked) formation, adenylylation, uridylylation, propionylation, pyroglutamate formation, S-glutathionylation, S-nitrosylation, S-sulfenylation, S-sulfinylation, S-sulfonylation, myristoylation, palmitoylation, stearoylation, isoprenylation/prenylation, farnesylation, geranilgeranilatyon, glipyatyon, glycosylphosphatidylinositol (GPI) anchor formation, lipoylation, disulphide bond(s) formation between two or more cysteine residues in the amino acid sequence, covalent binding of one or more lipid moieties to one or more amino acid residues, phosphopantetheinylation, one or more flavin moietys (FMN or FAD) may be covalently attached, heme C attachment via thioether bonds with cysteines, retinylidene Schiff base formation, ubiquitination, SUMOylation, one or more of a myristate, palmitate, farnesyl, geranyl-geranyl, GPI-anchor, N-acyl diglyceride group attached to one or more amino acid residues, a myristate group attached through an amide bond to the N-terminal glycine residue of the mature form of a protein and/or to an internal lysine residue(s), palmitate group attached through a thioether bond to one or more cysteine residues or through an ester bond to one or more serine and/or threonine residues, farnesyl group attached through a thioether bond to one or more cysteine residues, geranyl-geranyl group attached through a thioether bond to one or more cysteine residues, glycosyl-phosphatidylinositol (GPI) group linked to the alpha-carboxyl group of the C-terminal residue of the mature form of a protein, N-terminal cysteine of the mature form of a lipoprotein with an amide-linked fatty acid and a glyceryl group to which two fatty acids are linked by ester linkages, non-covalent complexation with pyrene butyrate.
(35) Modification of amino acids such as cysteine, lysine, glutamate/aspartate and tyrosine with suitable amine, thiol, carboxylic acid and phenol-reactive reagents so as to functionalize said amino acids, and introduction or replacement of amino acids that introduce orthogonal reactivities that are suitable for functionalization, for example azide or alkyn-group bearing amino acids that allow functionalization with alkyn or azide-bearing moieties, respectively. Post-synthetic bioorthogonal modification of the amino acids cysteine, lysine, glutamate and/or tyrosine with suitable amine, thiol, carboxylic acid and phenol-reactive reagents.
(36) A cysteine analogue can be used in place of cysteine at one or more places. Particular cysteine analogues include D-cysteine, homocysteine, alpha-methyl cysteine, mercaptopropionic acid, mercaptoacetic acid, penicillamine, acetylated forms of those analogues capable of accepting an acetyl group, acetylated homocysteine, acetylated penicillamine, acetylated alpha-methyl cysteine, cysteine analogue modified with blocking group(s).
(37) Selenocysteine is an amino acid which has a similar reactivity to cysteine and can be used for some the same reactions. Thus, wherever cysteine is mentioned/presented herein, in alternative embodiments, it is typically acceptable to substitute selenocysteine unless the context suggests otherwise.
(38) In some embodiments, one or more tryptophan residues are replaced with (independently selected at each point of replacement) a naphthylalanine or alanine residue, or an amino acid residue in the equivalent position in IF1 protein of a different species, which can improve the pharmaceutical stability profile.
(39) In some embodiments, an N-terminal and/or C-terminal modification comprises the addition (optionally via a spacer/linker region[s]) of an effector/functional group(s), including (but not limited to) a cell penetrating moiety/moieties, cytotoxic agent(s), drug(s), radiochelator(s) {which is suitable for complexing metal radioisotope(s) of medicinal relevance; such effectors, when complexed with said radioisotopes, can present useful agents for cancer therapy etc.}, chromophore(s), enzyme(s), antibody[y/ies] and/or binding fragments thereof (e.g. binding of which can increase half-life in vivo).
(40) One or more of the following substitutions is made at one or more places (wherein each substitution is independently selected from [if applicable] introducing the D- or L-form, wherein introduction of the L-form is favoured in some embodiments): Aspartic Acid (Asp) for Asparagine (Asn) or vice-versa; Glutamic Acid (Glu) for Glutamine (Gln) or vice-versa; 2-Aminoadipic acid (Aad) for Aspartic acid (Asp) or Glutamic acid (Glu) or other amino acid; 3-Aminoadipic acid (bAad) for Aspartic acid (Asp) or Glutamic acid (Glu) or other amino acid; beta-Alanine (bAa) for Glycine, Alanine (Ala), Valine (Val), Isoleucine (Ile), Leucine (Leu) or other amino acid; 2-Aminobutyric acid (Abu) for Glycine (Gly), Alanine (Ala), Valine (Val), Isoleucine (Ile), Leucine (Leu) or other amino acid; 4-Aminobutyric acid (4Abu) for Glycine (Gly), Alanine (Ala), Valine (Val), Isoleucine (Ile), Leucine (Leu) or other amino acid; 6-Aminocaproic acid (Acp) for Glycine (Gly), Alanine (Ala), Valine (Val), Isoleucine (Ile), Leucine (Leu) or other amino acid; 2-Aminoheptanoic acid (Ahe) for Glycine (Gly), Alanine (Ala), Valine (Val), Isoleucine (Ile), Leucine (Leu) or other amino acid; 2-Aminoisobutyric acid (Aib) for Glycine (Gly), Alanine (Ala), Valine (Val), Isoleucine (Ile), Leucine (Leu) or other amino acid; 3-Aminoisobutyric acid (bAib) for Glycine (Gly), Alanine (Ala), Valine (Val), Isoleucine (Ile), Leucine (Leu) or other amino acid; 2-Aminopimelic acid (Apm) for Aspartic acid (Asp) or Glutamic acid (Glu) or other amino acid; 2,4 Diaminobutyric acid (Dbu) for Arginine (Arg) or Histidine (His) or Lysine (Lys) or other amino acid; 2,2-Diaminopimelic acid (Dpm) for Aspartic acid (Asp) or Glutamic acid (Glu) or Asparagine (Asn) or Glutamine (Gln) or other amino acid; 2,3-Diaminopropionic acid (Dpr) for Arginine (Arg) or Histidine (His) or Lysine (Lys) or other amino acid; N-Ethylglycine (EtGly) for Glycine (Gly) or other amino acid; N-Ethylasparagine (EtAsn) for Asparagine (Asn) or other amino acid; Hydroxylysine (Hyl) for Lysine (Lys) or other amino acid; allo-Hydroxylysine (aHyl) for Lysine (Lys) or other amino acid; 3-Hydroxyproline (3Hyp) for Proline (Pro) or other amino acid; 4-Hydroxyproline (4Hyp) for Proline (Pro) or other amino acid; allo-Isoleucine (aIle) for Glycine (Gly), Alanine (Ala), Valine (Val), Isoleucine (Ile), Leucine (Leu) or other amino acid; N-Methylglycine/sarcosine (MeGly) for Glycine (Gly), Alanine (Ala), Valine (Val), Isoleucine (Ile), Leucine (Leu) or other amino acid; N-Methylisoleucine (MeIle) for Glycine (Gly), Alanine (Ala), Valine (Val), Isoleucine (Ile), Leucine (Leu) or other amino acid; 6-N-Methyllysine (MeLys) for Arginine (Arg) or Histidine (His) or Lysine (Lys), Glycine (Gly), Alanine (Ala), Valine (Val), Isoleucine (Ile), Leucine (Leu) or other amino acid; N-Methylvaline (MeVal) for Glycine (Gly), Alanine (Ala), Valine (Val), Isoleucine (Ile), Leucine (Leu) or other amino acid; Norvaline (Nva) for Glycine (Gly), Alanine (Ala), Valine (Val), Isoleucine (Ile), Leucine (Leu) or other amino acid; Norleucine (Nle) for Glycine (Gly), Alanine (Ala), Valine (Val), Isoleucine (Ile), Leucine (Leu) or other amino acid; Ornithine (Orn) for Arginine (Arg) or Histidine (His) or Lysine (Lys) or other amino acid.
(41) Substitution/insertion/deletion of one or more amino acids.
(42) Proteolytic/chemically cleaved at one or more places.
(43) Covalent attachment of a molecule/protein/ligand.
(44) Modified at one or more places with a peptide/protein modification(s) of the art.

### Peptide/Protein Synthesis

All amino acid sequences described herein can be prepared by chemical synthesis using either automated or manual solid phase synthetic technologies, known in the art. In some embodiments, peptides are produced by Fmoc synthesis, analysed by mass spectrometry, and purified by HPLC, all clear to those of the art (e.g. refer U.S. Pat. No. 8,080,517B2). How to synthesize an amino acid or nucleotide sequence is well known in the art. For non-limiting example, an amino acid sequence can be synthesized in-house using a fully automatic peptide synthesizer (e.g. model 431A of Applied Biosystems, e.g. a peptide synthesizer from Gyros Protein Technologies, Tucson, AZ, USA, e.g. “Liberty Blue” Automated Microwave Peptide Synthesizer from CEM Corporation, Matthews, NC, USA) or sourced from a protein synthesis company e.g. GenScript, Piscataway, NJ, USA, e.g. Biomatik, Kitchener, Ontario, Canada, e.g. Peptide 2.0, Chantilly, VA, USA, e.g. Bachem AG, Bubendorf, Switzerland, e.g. LifeTein, Somerset, New Jersey, e.g. Cambridge Research Biochemicals, Billingham, Cleveland, UK, e.g. Pepscan, Lelystad, Netherlands.

Some useful resources for methods of solid phase peptide/protein synthesis: Stewart M and Young J D (1984) “Solid Phase Peptide Synthesis, 2nd edition” Pierce Chemical Company, Rockford, Ill; Bodanzsky M and Bodanzsky A (1984) “The practice of Peptide Synthesis” Springer Verlag, NY; Lloyd-Williams P, Albericio F and Giralt E (1997) “Chemical Approaches to the Synthesis of Peptides and Proteins” CRC, Boca Raton, Fla., USA, Atherton B and Sheppard RC (1989) “Solid Phase Peptide Synthesis: A practical approach” IRL Oxford University Press. Some different/variant methods of peptide protein synthesis are disclosed in (for example) Lloyd Williams P, Albericio F, Giralt E (1993) “Convergent solid phase peptide synthesis” Tetrahedron 49:11065-11133; Kullmann W (1980) “Proteases as catalysts for enzymic syntheses of opioid peptides” J. Biol. Chem. 255:8234-8238; Smith M B, March J “March's Advanced Organic Chemistry Reactions, Mechanisms and Structure”, John Wiley & Sons, NJ, USA.

For example, a non-limiting method of obtaining a peptide/protein of this disclosure comprises the steps of:
coupling an amino acid with the N-terminal end protected and the C-terminal end free, on to an amino acid with the N-terminal end free and the C-terminal end protected or bound to a solid support;
removing the protective group of the N-terminal end;
repetition of the sequence of coupling and removal of the protective group of the N-terminal end until the desired peptide/protein sequence is obtained;
removal of the protective group of the C-terminal end or cleavage from the solid support.

Preferably, the C-terminal end is bound to a solid (e.g. polymer) support and the process is conducted on solid phase.

During the above, the functional groups of the side chains of the amino acids are adequately protected with temporary/permanent protective groups, and are de-protected simultaneously, or orthogonally, to the process of cleaving the completed peptide/protein from the solid support.

Examples of different protecting groups that can be used during peptide/protein synthesis for each of the N-terminus (e.g. an amide, or e.g. Boc or Fmoc), C-terminus (e.g. an ester), and for different functional groups of different amino acid side-chains can be found in (for example) U.S. Pat. No. 8,946,166B2 (which teaches methods of peptide/protein synthesis), and references therein: e.g. Greene T W and Wuts P G M (1999) “Protective groups in organic synthesis” John Wiley & Sons, NY, USA, wherein U.S. Pat. No. 8,946,166B2 also discloses materials (e.g. polymeric), with supporting references, that can be used as the solid support. Optional modification of the N-terminal and C-terminal ends can be conducted whilst the peptide/protein is still bound to the solid support or after it has been cleaved off.

Alternatively, an amino acid sequence(s) (peptide[s]/protein[s]) of this disclosure can be produced by a recombinant method(s) of the art, optionally utilizing E. Coli or Saccharomyces cerevisiae.

Some Illustrative Polynucleotide Sequences of this Disclosure

DNA sequence encoding SEQ ID NO:130 (example epitope tag) concatenated to DNA sequence encoding SEQ ID NO:461 (RRRRRRRG, example CPP sequence) concatenated to DNA sequence encoding SEQ ID NO:162 (human Mitochondrial Import Sequence [MIS] for its IF1 protein) concatenated to DNA sequence encoding complete “mature” (without MIS) IF1 protein from bowhead whale (using “mature” [without MIS] IF1 protein numbering: residues 1-82 of bowhead whale IF1 protein; residues 26-107 of SEQ ID NO:336), with codon selection optimized for the codon bias of E. coli (SEQ ID NO:1426), S. cerevisiae (SEQ ID NO:1427), or Homo sapiens (SEQ ID NO:1428). SEQ ID NO:1429 to SEQ ID NO:1446 follow this same pattern, showing different DNA sequences optimized for expression in those different species, but wherein the encoded bowhead whale IF1 protein component is instead, in turn, residues (using “mature” [without MIS] IF1 protein numbering) 1-60, 10-60, 14-60, 13-47, 14-47, 42-58 of bowhead whale IF1 protein. SEQ ID NO:1447 to SEQ ID NO:1467, and SEQ ID NO:1468 to SEQ ID NO:1488, are each the same as SEQ ID NO:1426 to SEQ ID NO:1446 respectively except that blue whale and human IF1 protein respectively are encoded instead. SEQ ID NO:1489 to SEQ ID NO:1500 are the same as SEQ ID NO:1426 to SEQ ID NO:1437 respectively except that these sequences confer (using “mature” [without MIS] IF1 protein numbering) a H49K substitution in their encoded IF1 protein/fragment component. SEQ ID NO:1501 to SEQ ID NO:1518 are the same as SEQ ID NO:1447 to SEQ ID NO:1464 respectively except that these sequences confer (using “mature” [without MIS] IF1 protein numbering) T14A and H49K substitutions in their encoded IF1 protein/fragment component. SEQ ID NO:1519 to SEQ ID NO:1536 are the same as SEQ ID NO:1468 to SEQ ID NO:1485 respectively except that these sequences confer (using “mature” [without MIS] IF1 protein numbering) S14A and H49K substitutions in their encoded IF1 protein/fragment component. SEQ ID NO:1537 to SEQ ID NO:1647 are the same as SEQ ID NO:1426 to SEQ ID NO:1536 respectively except that a different CPP sequence is encoded instead (a Tat sequence flanked by glycines, GYGRKKRRQRRRG, SEQ ID NO:445), the encoded MIS is that for mouse's IF1 protein instead (SEQ ID NO:163), and codon selection is optimized for the codon bias of Mus musculus instead of Homo sapiens. SEQ ID NO:1648 to SEQ ID NO:1683 are the same as SEQ ID NO:1426, SEQ ID NO:1427, SEQ ID NO:1428, SEQ ID NO:1447, SEQ ID NO:1448, SEQ ID NO:1449, SEQ ID NO:1468, SEQ ID NO:1469, SEQ ID NO:1470, SEQ ID NO:1468, SEQ ID NO:1469, SEQ ID NO:1470, SEQ ID NO:1489, SEQ ID NO:1490, SEQ ID NO:1491, SEQ ID NO:1501, SEQ ID NO:1502, SEQ ID NO:1503, SEQ ID NO:1519, SEQ ID NO:1520, SEQ ID NO:1521, SEQ ID NO:1537, SEQ ID NO:1538, SEQ ID NO:1539, SEQ ID NO:1558, SEQ ID NO:1559, SEQ ID NO:1560, SEQ ID NO:1579, SEQ ID NO:1580, SEQ ID NO:1581, SEQ ID NO:1600, SEQ ID NO:1601, SEQ ID NO:1602, SEQ ID NO:1612, SEQ ID NO:1613, SEQ ID NO:1614, SEQ ID NO:1630, SEQ ID NO:1631, SEQ ID NO:1632 respectively except that they display a consensus DNA sequence, encompassing the use of a number of codons at a number of positions, given the codon bias of the species, rather than just a single codon at each position. From these presented consensus DNA sequences one of the art can arrive at consensus sequences for the other aforementioned DNA sequences, for which a consensus sequence is not presented. Any of SEQ ID NO:1426 to SEQ ID NO:1536 can have its coding region for the CPP component, and/or coding region for the MIS component, substituted with the CPP component, and/or MIS component, coding regions of SEQ ID NO:1537. Similarly, any of SEQ ID NO:1537 to SEQ ID NO:1647 can have its coding region for the CPP component, and/or coding region for the MIS component, substituted with the CPP component, and/or MIS component, coding regions of SEQ ID NO:1426. These DNA sequences but with a different CPP component coding region, coding for a different CPP, optionally coding for one or more of SEQ ID NO:124 to SEQ ID NO:126 (and/or one or more of SEQ ID NO:440 to SEQ ID NO:638), and/or with a different MIS coding region (e.g. coding for an MIS from a different species), and/or with a different IF1 protein/fragment (or sequence variant thereof) coding region (e.g. coding for that from a different species and/or a different fragment thereof, comprising a different residues range) are hereby contemplated, as are their encoded amino acid sequences, and peptides/proteins thereof. Any sequence selected from SEQ ID NO:1426 to SEQ ID NO:1536 with a part of it substituted with the exactly corresponding part (same range of nucleotide position numbers) of a different sequence selected from SEQ ID NO:1426 to SEQ ID NO:1536, but preferably with the caveat that only whole codons are substituted (no substitution if one or both the ends of the substituted sequence fall within a codon), and wherein the modified (if applicable) protein sequence encoded is also componentry to this disclosure. This affords the possibility of the IF1 protein/fragment (or sequence variant thereof) component to be a fusion of that from two or more of human, blue and bowhead whale. For example, wherein the IF1 protein/fragment (or sequence variant thereof) component is (using “mature” [without MIS] IF1 protein numbering), up to the encoded 47^{th} amino acid residue, from human or blue whale, and its remainder is from bowhead whale, wherein the amino acid sequence of this novel human or blue whale and bowhead whale IF1 protein/fragment (or sequence variant thereof), as a separate sequence embodiment, is also componentry to this disclosure. Any sequence selected from SEQ ID NO:1537 to SEQ ID NO:1647 with a part of it substituted with the exactly corresponding part (same range of nucleotide position numbers) of a different sequence selected from SEQ ID NO:1537 to SEQ ID NO:1647, but preferably with the caveat that only whole codons are substituted (no substitution if one or both the ends of the substituted sequence fall within a codon), and wherein the modified (if applicable) protein sequence encoded is also componentry to this disclosure (and partially/completely retroinverse sequence thereof). To illustrate, and not restrict, residues 1-282 of any one of SEQ ID NO:1468, SEQ ID NO:1469, SEQ ID NO:1470, SEQ ID NO:1471, SEQ ID NO:1472, SEQ ID NO:1473, SEQ ID NO:1519, SEQ ID NO:1520, SEQ ID NO:1521, SEQ ID NO:1522, SEQ ID NO:1523, SEQ ID NO:1524, SEQ ID NO:1447, SEQ ID NO:1448, SEQ ID NO:1449, SEQ ID NO:1450, SEQ ID NO:1451, SEQ ID NO:1452, SEQ ID NO:1501, SEQ ID NO:1502, SEQ ID NO:1503, SEQ ID NO:1504, SEQ ID NO:1505, SEQ ID NO:1506, SEQ ID NO:1426, SEQ ID NO:1427, SEQ ID NO:1428, SEQ ID NO:1429, SEQ ID NO:1430, SEQ ID NO:1431, concatenated to residues 285-387 or residues 285-321 of any one of SEQ ID NO:1426, SEQ ID NO:1427, SEQ ID NO:1428, SEQ ID NO:1489, SEQ ID NO:1490, SEQ ID NO:1491, SEQ ID NO:1447, SEQ ID NO:1448, SEQ ID NO:1449, SEQ ID NO:1501, SEQ ID NO:1502, SEQ ID NO:1503, wherein the encoded protein sequences are also componentry to this disclosure (and fragments, and concatenated fragments, thereof e.g. without one or more of the epitope/affinity tag, CPP, MIS components; and/or partially/completely retroinverse sequence thereof). By the teaching of the last sentence, equivalent modifications can be made for sequences within the range SEQ ID NO:1537 to SEQ ID NO:1647 (but with different residue numbering because their CPP component is a longer sequence). Fragments (or concatenated fragments) of any of the aforementioned DNA sequences (and their encoded amino acid sequences) are contemplated, for example any of SEQ ID NO:1426 to SEQ ID NO:1683 truncated at their 5′ terminal end by 42 nucleotides (such that they don't encode the epitope/affinity tag component), or truncated further such that they also don't encode the Cell Penetrating Peptide (CPP) component, or truncated even further such that they also don't encode the MIS component. Any of the aforementioned nucleotide/amino acid sequences with one or more of the regions coding for the epitope/affinity tag, Cell Penetrating Peptide (CPP), and MIS components absent are componentry to this disclosure. Not shown, but contemplated, is any of the aforementioned DNA sequences with a stop codon (e.g. tag, taa, tga) added at their 3′ terminal end, and optionally with a termination sequence thereafter (e.g. enables release of RNA polymerase during transcription), wherein any termination sequence of the art is contemplated, including thymine rich region(s) (poly-T tail), cytosine rich region(s), palindromic region(s) etc, wherein illustratively SEQ ID NO:1684 is the termination sequence of the human ATP5IF1 gene. One or more of these DNA sequences, but with their codon use optimized for a different species, is componentry to this disclosure (e.g. by inputting the encoded protein sequence, along with codon bias data for the selected species sourced from https://www.kazusa.or.jp/codon/, into https://www.bioinformatics.org/sms2/rev_trans.html). Also contemplated is for one or more of these DNA sequences to be expressed in mitochondria, instead of transcribed in the nucleus (and translated in the cytoplasm), and to have its codons selected accordingly (mitochondria use slightly different genetic code than nucleus). For a peptide/protein of/in this disclosure, all DNA sequences that code for it by a genetic code, and the mRNA sequences transcribed therefrom, are componentry to this disclosure (as are the sequences of complimentary/base-paired non-coding strands, optionally existing as double-stranded). Given that typically in a genetic code there is more than one codon coding for each amino acid, the number of nucleotide sequences disclosed herein is too great to list, but wherein nonetheless they are all componentry to this disclosure. To illustrate, using the standard genetic code, there are 3.24357*10⁵⁰ DNA sequences coding for the amino acid sequence of human “immature” (with MIS) IF1 protein, and 2.16238*10⁵⁰ DNA sequences coding for the amino acid sequence of human “mature” (without MIS) IF1 protein.

Polynucleotide comprising (or consisting of) one or more sequences selected from SEQ ID NO:1426 to SEQ ID NO:1684. Peptide/protein comprising (or consisting of) one or more amino acid sequences encoded by SEQ ID NO:1426 to SEQ ID NO:1684 (or fragment thereof, or concatenated fragments thereof, and/or partially/completely retroinverse sequence thereof).

### Polynucleotide Derivative(s) Thereof

Contemplated by, and componentry to, this disclosure is any nucleotide sequence of/in this disclosure, wherein one or more of the nucleotides are modified with a polynucleotide modification of the art (and when a nucleotide sequence is referred to herein, in alternative embodiments, this referral is to the sequence modified such that one or more of the following apply to it), for non-limiting example, by methylation at one or more places, by addition of a carboxymethylaminomethyl group, addition of a methoxy group etc. For non-limiting example, wherein, at one or more places, a guanosine motif in the polynucleotide sequence is substituted with a group independently selected at each site from queuosine, D-galactosylqueuosine, D-mannosylqueuosine, 2′-O-methylguanosine, 1-methylguanosine, 2,2-dimethylguanosine, 2-methylguanosine, 7-methylguanosine; and/or wherein, at one or more places, an adenosine motif in the polynucleotide sequence is substituted with a group independently selected at each site from inosine, 1-methylinosine, N6-isopentenyladenosine, 1-methyladenosine, 2-methyladenosine, N6-methyladenosine, 2-methylthio-N6-isopentenyladenosine; a cytidine motif in the polynucleotide sequence is substituted with a group independently selected at each site from 4-acetylcytidine, 2′-O-methylcytidine, 3-methylcytidine, 5-methylcytidine, methyl-cytosine, 2-thiocytidine; a uridine/thymidine motif in the polynucleotide sequence is substituted with a group independently selected at each site from 5-(carboxyhydroxymethyl) uridine, 5-carboxymethylaminomethyl-2-thiouridine, 5-carboxymethylaminomethyluridine, dihydrouridine, 2′-O-methylpseudouridine, 1-methylpseudouridine, 5-methylaminomethyluridine, 5-methoxyaminomethyl-2-thiouridine, 5-methoxycarbonylmethyl-2-thiouridine, 5-methoxycarbonylmethyluridine, 5-methoxyuridine, uridine-5-oxyacetic acid-methylester, uridine-5-oxyacetic acid, pseudouridine, 5-methyl-2-thiouridine, 2-thiouridine, 4-thiouridine, 5-methyluridine, 2′-O-methyl-5-methyluridine, 2′-O-methyluridine, 3-(3-amino-3-carboxy-propyl) uridine, (acp3)u. A good related resource is: Protocols for Oligonucleotides and Analogs. Synthesis and Properties, Agrawal, ed., Methods in Molecular Biology Vol. 20, Humana Press, Totowa, N.J.

As used herein, the terms “nucleic acid(s)” and “nucleotide sequence(s)” and “polynucleotide(s)” include polymeric form of nucleotides of any length, deoxyribonucleotides, ribonucleotides, DNA molecules (e.g. cDNA or genomic DNA), RNA molecules (e.g. mRNA), combinations of DNA and RNA molecules or hybrid DNA/RNA molecules, and analogs, derivatives and modifications (e.g. methylation) of DNA or RNA molecules. Analogs can be generated using, for example, nucleotide analogs, which include, but are not limited to, inosine or tritylated bases. Such analogs can also comprise DNA or RNA molecules comprising (or consisting of) modified backbones that lend beneficial attributes to the molecules such as, for example, nuclease resistance or an increased ability to cross cellular membranes. The nucleic acids or nucleotide sequences can be single-stranded (the sense or antisense strand), double-stranded, may contain both single-stranded and double-stranded portions, and may contain triple-stranded portions.

### Vector(s) Thereof of the Disclosure

In some embodiments, a vector/“expression vector” herein is a nucleotide sequence that contains at least one nucleotide sequence(s) of/in this disclosure, optionally a double-/single-stranded (or mixed) circular/linear (or mixed) DNA/RNA (or mixed), comprising at least one nucleotide sequence of/in this disclosure, optionally to be transferred into a host cell and/or into a unicellular/multicellular host organism, wherein the expression vector contains appropriate regulatory element(s), some non-limiting examples of which are given in U.S. Pat. No. 8,080,517B2 and/or [P14] (and references therein), to support the expression of said nucleotide sequence(s) of/in this disclosure inside a cell (preferably wherein the regulatory element(s) selected is tailored to the cell species/type in which the expression vector may be expressed). Appropriate cell lines or host systems may be chosen to ensure the desired modification and processing of a foreign peptide/protein is achieved. For example, peptide expression within a bacterial system can be used to produce a non-glycosylated core peptide; whereas expression within mammalian cells ensures “native’ glycosylation of a heterologous peptide. An introduction to this area is given by {for non-limiting example} Goeddel: Gene expression technology: Methods in Enzymology, Academic Press, San Diego, California; Sambrook et al.: Molecular cloning: a laboratory manual, Cold Spring Harbor Laboratory Press, Cold Spring Harbor, NY, USA; and Short Protocols in Molecular Biology (1999), John Wiley & Sons Inc, NJ, USA. Note that a strategy to maximize recombinant protein expression in E. coli is to express the protein in a host bacteria with an impaired capacity to proteolytically cleave the recombinant protein. Another strategy is to design the nucleic acid sequence of the nucleic acid to be inserted into an expression vector so that the individual codons for each amino acid are those preferentially utilized in E. coli (Wada et al., (1992) Nucleic Acids Res. 20:2111-2118) and/or use a Rosetta strain of E. coli, which expresses a variety of tRNAs that are normally found at low abundance in E. coli. Note that, in an alternative, a recombinant expression vector can be transcribed and translated in vitro, for example using T7 promoter regulatory sequences and T7 polymerase.

An expression vector comprising at least one DNA sequence of/in this disclosure operably linked to an expression control sequence; a cultured cell comprising this vector. A cultured cell comprising at least one DNA sequence of/in this disclosure operably linked to an expression control sequence; a method of making a peptide/protein of/in this disclosure comprising culturing the cell under conditions permitting expression of the aforementioned DNA sequence. A vector comprising a nucleotide sequence encoding at least one amino acid sequence of/in this disclosure. DNA comprising a sequence encoding at least one amino acid sequence of/in this disclosure, wherein the sequence is operably linked to a heterologous expression control sequence. At least one nucleotide sequence, preferably DNA, of/in this disclosure as part of a longer nucleotide sequence that comprises one or more of a “start” (translation initiating) codon, a “stop” (translation terminating) codon, termination sequence, gene expression control element(s), regulatory DNA sequence(s), promoter (constitutive or inducible, optionally tissue(s) specific), enhancer(s), inducer(s), transcription/translation termination sequence(s), ribosomal binding site(s), regulatory DNA sequence(s), leader sequence(s), 5′ and 3′ untranslated regions, 3′-UTR sequence that contains a polyadenylation sequence, transcriptional/translation control region(s), Kozak sequence, amplification marker(s)/reporter(s), antibiotic resistance gene(s), reporter gene(s), tag(s), fusion partner sequence(s) is componentry to this disclosure. At least one nucleotide sequence of/in this disclosure incorporated into a longer nucleotide sequence/genome/chromosome/vector/virus/plasmid/episome/expression vector/recombinant vector, optionally inside a host cell (e.g. in unicellular microorganism species or human/animal derived cell line or Embryonic Stem [ES] cell(s) or in one or more cells of a multicellular animal), is componentry to this disclosure. Optionally wherein the vector can be a nucleotide sequence that contains/comprises at least one nucleotide sequence of/in this disclosure. Optionally wherein the vector is/comprises at least one nucleotide sequence of/in this disclosure and one or more of a gene expression control element(s), promoter(s) (constitutive or inducible, optionally tissue(s) specific), enhancer(s), inducer(s), termination sequence(s), regulatory DNA sequence(s), antibiotic resistance gene(s), reporter gene(s), optionally wherein the vector is a virus (optionally a recombinant adeno-associated virus [AAV]) or plasmid, capable or incapable of replication inside a host cell, preferably wherein the vector in a host cell leads to the expression of one or more amino acid sequence embodiments of/in the disclosure in this host cell. Many applicable vectors are known to those of the art.

In some embodiments, the expression vector also expresses something that makes it “selectable” e.g. expresses a gene conferring antibiotic/drug/toxin resistance and/or confers ability to survive on a medium that the cell(s) wouldn't be able to if it didn't have, and wasn't expressing, the expression vector etc. A particular vector can be made with a particular target cell type(s)/population of cells/tissue(s) in mind. For example, particular regulatory elements, such as control elements and promoters, can be chosen based on the target cells, such that the regulatory elements are operable in the target cells.

### Cell(s) Thereof of the Disclosure

A nucleic acid expression construct (“expression vector”), optionally capable of autonomous replication, encoding at least one amino acid sequence(s) of/in this disclosure (e.g. a plasmid, virus, cosmid, phagemid etc.). A host cell(s) (e.g. [non-limiting] microoganism/prokaryote/eukaryote/bacteria/yeast/mammalian/insect/plant cell(s)/cell line/E. coli/Saccharomyces cerevisiae etc., optionally in vitrolin vivolex vivo) comprising this expression construct. A method for producing an amino acid sequence(s) of/in this disclosure comprising culturing this host cell(s), under conditions suitable for the expression of the amino acid sequence(s), and recovering said amino acid sequence(s) therefrom. Non-limiting methods are described in U.S. Pat. No. 5,906,923 (ATPASE INHIBITOR, Jennifer L. Hillman), herein incorporated in its entirety by reference. Other methods are known to those of the art e.g. refer to [P14], herein incorporated in its entirety by reference, and references therein.

Componentry to this disclosure is to produce an amino acid sequence(s) of/in this disclosure recombinantly.

A method comprising: (i) culturing a host cell comprising an expression vector comprising a nucleotide sequence encoding at least one amino acid sequence of/in this disclosure under conditions which provide for expression of said amino acid sequence(s), encoded by the expression vector within the host cell; and recovering the amino acid sequence(s), optionally by way of an epitope/affinity tag sequence component, optionally wherein this tag is then removed, optionally by the epitope/affinity tag sequence being connected to one end of the amino acid sequence(s), optionally the N-terminal end, by a cleavable linker sequence that is cleaved.

A process for the manufacture of at least one amino acid sequence of/in this disclosure by culturing a micro-organism that produces it in a nutrient medium.

An expression vector comprising a nucleotide sequence encoding at least one amino acid sequence of/in this disclosure. A transformant obtained by transforming a host with this expression vector. A method for manufacturing an amino acid sequence(s) of/in this disclosure, comprising culturing the transformant to produce it; and recovering the produced amino acid sequence(s) from its culture.

A cell line comprising (or consisting of) cells transfected with a polynucleotide comprising at least one nucleotide sequence of/in this disclosure.

A vector comprising a nucleotide sequence encoding at least one amino acid sequence on this disclosure.

A cell(s) transfected with a DNA/RNA encoding a polypeptide/peptide of/in this disclosure and expressing this polypeptide/peptide.

A cell of this disclosure fused with a different cell of this disclosure, and/or a cell not of this disclosure, is a cell of this disclosure.

### Transgenic Organism(s) Thereof of the Disclosure

A transgenic organism(s)/subject(s), optionally a non-human transgenic animal and/or a non-human transgenic mammal, optionally a microorganism, with one or more nucleotide sequences of/in this disclosure incorporated into one or more of their cells, optionally incorporated into the genome (nuclear and/or mitochondrial genome, preferably stably, if in mitochondria: codon usage is preferably optimized for mitochondrial variant, for that species, of genetic code) of one or more of their cells, optionally wherein the modified cells include germ cells, optionally wherein this subject is a test subject in a lifespan/healthspan/brainspan study. The transgenic subject can be a human or, in alternative embodiments, any organism, preferably an animal, that isn't human. In some embodiments it is a multicellular organism. In some embodiments it is a laboratory animal, such as (without limitation) a mouse (especially preferred) or rat or other rodent. In some embodiments it is a pet/companion animal, such as (without limitation) a dog or cat. In some embodiments it is a commercial/farm animal, such as (without limitation) a cow, pig or racehorse. In some embodiments it is a microorganism/unicellular organism, such as (without limitation) a yeast or bacterium. A transgenic non-human animal, preferably a transgenic non-human mammal, which comprises a gene/nucleotide sequence encoding at least one amino acid sequence of/in this disclosure stably integrated into its genome. Method of producing an animal, optionally a mammal, preferably a non-human mammal, optionally a mouse, with a longer healthspan and/or lifespan by introducing a vector(s) comprising at least one nucleotide sequence of/in this disclosure.

In some embodiments, a nucleotide sequence(s) of/in this disclosure is expressed in all the cells of the subject, optionally because it is under the control of (operably linked to) a constitutive promoter(s)/regulatory element(s). In other embodiments, a nucleotide sequence(s) of/in this disclosure is expressed in some of the cells of the subject, optionally delimited to cells of a particular cell type(s), tissue(s), organ(s), body part(s) etc., optionally because it is under the control of (operably linked to) a cell type(s)/tissue(s)/organ(s) specific promoter(s)/regulatory element(s). In some embodiments, a nucleotide sequence(s) of/in this disclosure is expressed in one or more cells of the subject only when it is induced to do so by an inducible promoter(s)/regulatory element(s). In some embodiments, a nucleotide sequence(s) of/in this disclosure is inheritable e.g. it can be found in one or more offspring of the subject, preferably who themselves can pass it on to one or more of their offspring.

In some embodiments, a nucleotide sequence(s) of/in this disclosure is “knocked-in” (optionally using homologous recombination) to their native IF1 gene, replacing part(s) or totality thereof. Optionally wherein the “knock-in” is an IF1 gene from a different, preferably bigger and/or longer living, species and/or wherein the “knock-in” is a mutated/variant IF1 gene with a modified “phosphorylation control switch” residue (replaced with an amino acid residue that cannot be phosphorylated) and/or an attenuated “pH dependence motif” (FIG. 10) and/or a truncated IF1 gene e.g. without a “pH dependence motif” (e.g. expressing only IF1 protein residues 14-47, using “mature” bovine numbering, and/or the equivalent/best aligned component of a different IF1 protein) and/or some other sequence change(s) mentioned herein and/or multiple (optionally different from one another) IF1 gene (and/or mutant(s)/variant(s) thereof) sequences are knocked in place of one, to increase the amount of IF1 protein expressed, and/or sequence change(s) is made to the promoter(s)/regulatory region(s) of the IF1 gene (and/or mutant(s)/variant(s) thereof) to increase its expression.

How to produce a transgenic organism is well known to those of the art e.g. refer to [P14] and references therein, including U.S. Pat. Nos. 5,675,060, 5,850,001, 5,792,902, 5,573,933, 5,633,076, 5,741,957, 5,827,690, 5,831,141, 5,849,992, 5,814,300, WO94/24301; Watson et al., Recombinant DNA, W.H. Freeman & Co., New York, NY; Hogan, et al., Manipulating the Mouse Embryo, Cold Spring Harbor Press, Cold Spring Harbor, NY; Jaenisch, Proc. Natl. Sci. U.S.A. 73:1260-1264, (1976); Soriano and Jaenisch, Cell 46:19-29, (1986); Jahner et al., Proc. Natl. Acad. Sci. U.S.A. 82:6927-6931(1985); Van der Putten et al., Proc. Natl. Acad. Sci. U.S.A. 82:6148-6152(1985); Stewart, et al., EMBO J. 6:383-388(1987); Jahner et al., Nature 298:623-628(1982); Jaenisch, Science 240:1468-1474 (1988); Teratocarcinomas and Embryonic Stem Cells. A Practical Approach, Robertson E J, ed., IRL Press, Oxford); Pinkert, Transgenic Animal Technology, a Laboratory Handbook, Academic Press Inc., San Diego.

For example, a nucleotide sequence(s) (and/or vector(s) thereof) of/in this disclosure can be introduced into one or more embryonic cells. For non-limiting example,
(1) in vitro, a nucleotide sequence(s) of/in this disclosure (and/or vector(s) thereof) can be injected into a zygote [fertilized egg, in most cases all ensuing cells will then carry this injected nucleotide sequence(s) of the disclosure—so the ensuing animal is NOT a mosaic/chimera, with some of its cells carrying and others not] (Brinster et al., Proc. Natl. Acad. Sci. U.S.A. 82:4438-4442, 1985); or
(2) in vitro, blastomeres/blastocoeles infected with a virus(s) (e.g. retrovirus, preferably replication-defective, but can still perform viral integration into the genome) carrying a nucleotide sequence(s) of/in this disclosure (tends to produce mosaic/chimera animals, but if the nucleotide sequence(s) of/in this disclosure gets into germ cells, offspring can be produced with it in all of their cells; infection of blastomeres is enhanced by enzymatic removal of the Zona pellucida); or
(3) in vitro, a nucleotide sequence(s) of/in this disclosure (and/or vector(s) thereof) can be introduced into one or more Embryonic Stem (ES) cells (ES cells are obtained from pre-implantation embryos that are cultured in vitro) which can be combined with an animal blastocyst, after which the ES cells colonize the embryo and contribute to the germline of the resulting animal, which is a mosaic/chimera animal, from which an offspring animal with the nucleotide sequence(s) in all of its cells can be produced.

By the teaching of this disclosure, a transgenic animal (e.g. mouse) that has more IF1 protein, and/or a more potent IF1 protein(s)/fragment(s) (and/or sequence variant(s) thereof) {at pH 8} in all its cells (e.g. IF1 protein from a bigger and/or longer living species), will have a longer lifespan and/or healthspan than an unmodified animal of the same species, IF the transgenic animal is reared at a conducive ambient temperature to its lesser endogenic heat production, and/or is afforded extra bodily insulation. In other embodiments, a transgenic animal has more IF1 protein, and/or a more potent IF1 protein(s)/fragment(s) (and/or sequence variant(s) thereof) {at pH 8} in some of its cells, optionally in only a specific cell type(s)/tissue(s)/organ(s)/body part(s)/region(s), optionally only in one or more cells of the brain, preferably increasing “brainspan”, wherein heat transfer from other body regions (especially via blood flow), which don't have the reduced endogenic heat production, makes up for the reduced endogenic heat production in this area, which means the cold tolerability of the animal is closer to wild-type than the aforementioned animal, which has all of its cells modified with a nucleotide sequence(s) of/in this disclosure.

A method wherein the lifespan and/or healthspan and/or brainspan of a transgenic organism of this disclosure is assayed in an assay(s) thereof.

### Pharmaceutical/Cosmetic Composition(s) Thereof

All possible stereoisomers, and mixtures of stereoisomers thereof, of SEQ ID NO:X (or fragment thereof, or concatenated fragments thereof) are contemplated;
a pharmaceutically/cosmetically acceptable salt, solvate, hydrate, prodrug, liposome, nanoparticle (e.g. lipid nanoparticle, LNP) or other vector of the art of SEQ ID NO:X (or fragment thereof, or concatenated fragments thereof);
a medicament or a pharmaceutical/dermopharmaceutical/cosmetic/topical/supplement composition comprising (or consisting of) at least one peptide/protein of SEQ ID NO:X (or fragment thereof, or concatenated fragments thereof), and/or at least one pharmaceutically/cosmetically acceptable salt, solvate, hydrate, prodrug, liposome, nanoparticle (e.g. lipid nanoparticle, LNP) or other vector of the art thereof, and/or one or more polynucleotides [and/or at least one pharmaceutically/cosmetically acceptable salt, solvate, hydrate, prodrug, liposome, nanoparticle (e.g. lipid nanoparticle, LNP) or other vector of the art thereof] coding by the genetic code for at least one of SEQ ID NO:X {or fragment thereof, or concatenated fragments thereof}, and/or vector(s)/gene therap(y/ies) thereof and/or cell(s)/transgenic organism(s) thereof,
   and use thereof (optionally for at least one use disclosed herein), optionally for the treatment/amelioration/prevention/reversal/combat/slowing/delaying of cancer and/or aging (e.g. for preventative/therapeutic/aesthetic utility) and/or one or more diseases/disorders/physiological processes (and/or one or more of its consequences), or an unwanted/undesirable aesthetic(s), specified herein; wherein X can be 1, or the number of sequences in the Sequence Listing component to this application, or any integer between 1 and the total number of sequences in the Sequence Listing component to this application

Optionally administered via a protein therapy administration route/protocol of the art e.g. as used for an enzyme replacement therapy (ERT) e.g. intravenous infusion of protein(s) and/or medicament/composition/solution thereof.

A pharmaceutical composition comprising (or consisting of) at least one amino acid and/or nucleotide sequence (or vector(s) thereof, optionally wherein the vector is a plasmid or virus {optionally a recombinant adeno-associated virus, AAV}) of/in this disclosure and at least one of a pharmaceutically-acceptable carrier(s), additive(s), diluent(s), excipient(s). A pharmaceutical composition comprising (or consisting of) at least one peptide/protein comprising (or consisting of) at least one amino acid sequence of/in this disclosure, in a partially/completely bicyclic form, and at least one of a pharmaceutically-acceptable carrier(s), additive(s), diluent(s). Use of at least one amino acid and/or nucleotide sequence (or vector(s) thereof, optionally wherein the vector is a plasmid or virus {optionally a recombinant adeno-associated virus, AAV}), and/or pharmaceutical composition thereof, of/in this disclosure for the manufacture of a medicament. Use of at least one amino acid and/or nucleotide sequence (or vector(s) thereof, optionally wherein the vector is a plasmid or virus {optionally a recombinant adeno-associated virus, AAV}), and/or pharmaceutical composition thereof, of/in this disclosure for the manufacture of a medicament for treating/ameliorating/preventing/combating a disease/disorder/condition/process mentioned herein. Herein, wherever “pharmaceutical composition” is referred to, in other embodiments, this is substituted with “cosmetic composition” or “supplement composition”.

### Gene Therapy/Therapies

Use of at least one nucleotide sequence and/or at least one vector(s) thereof, optionally wherein the vector is a plasmid or virus/viral vector {optionally a recombinant adeno-associated virus, AAV}, optionally with one or more operably linked regulatory elements (e.g. a promoter and/or similar), of/in this disclosure for gene therapy. The vector is preferably a viral vector used in the art of gene therapy. Optionally derived from one or more of a retrovirus, adenovirus, adeno-associated virus, papilloma/herpes/vaccinia virus. Any method(s) relating to gene therapy available within the art may be used in the practice of the present disclosure (see e.g. Goldspiel et al., (1993) Clin Pharm 12:488-505; Larrick et al, Gene Therapy, Elsevier, NY, 1991).

Appropriate viral vectors can be selected based on the route of administration and the target cell type(s)/cell population(s). For example, retroviruses are preferred if the target cell type/population is actively proliferating and other viruses, such as lentivirus, adeno-associated virus, adenoviruses, are preferred if the target cell type/population is not actively proliferating. Different viruses have different specificity for different cell types and populations. Thus, a virus, or plural thereof, that infects a targeted cell type or population of cells can be selected.

Componentry to this disclosure is any nucleic acid sequence(s) that codes for one or more amino acid sequences of/in this disclosure. In specific embodiments, at least one nucleic acid sequence(s) encoding one or more amino acid sequence embodiments of/in this disclosure is administered to a subject, optionally to treat and/or enhance the subject, by way of gene therapy. Any method(s) for gene therapy available in the art can be used according to the present disclosure. For general reviews of the methods of gene therapy, refer Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May 1993, TIBTECH 11(5): 155-215. Methods known in the art of recombinant DNA technology can be used, some of which are described in Ausubel et al. (eds.), Current Protocols in Molecular Biology, John Wiley & Sons, NY (1993); and Kriegler, Gene Transfer and Expression, A Laboratory Manual, Stockton Press, NY (1990). In a preferred aspect, a pharmaceutical/cosmetic composition of this disclosure comprises at least one nucleic acid sequence, encoding at least one amino acid/peptide/protein sequence embodiment of/in this disclosure, in an expression vector, which (e.g. within a cell) expresses the nucleic acid sequence(s) into an amino acid/peptide/protein sequence(s) of/in this disclosure. Preferably such a nucleic acid sequence(s) has a promoter, optionally a heterologous promoter, operably linked to the protein coding region. Wherein said promoter is inducible or constitutive, and, optionally, tissue-specific. With an inducible promoter, expression of the nucleic acid sequence(s) is controllable by controlling the presence or absence of the appropriate inducer of transcription (non-limiting e.g. “Tet-Off” {transcription is inactive in presence of a tetracycline e.g. doxycycline or similar structure} and “Tet-On” {transcription is only active in presence of a tetracycline e.g. doxycycline or similar structure} expression systems). In particular embodiments, the nucleic acid sequence(s) used has the sequence coding for the therapeutic amino acid/peptide/protein sequence(s) embodiment, and optionally its regulatory region(s) {promoter(s) etc.}, flanked by regions that promote homologous recombination at a desired site in the genome, thus providing for intrachromosomal expression of the amino acid/protein sequence(s) encoding nucleic acid sequence(s) (Koller and Smithies, 1372718-2 72 1989, Proc. Natl. Acad. Sci. USA 86:8932-8935; Zijlstra et al., 1989, Nature 342:435 438). Delivery of the nucleic acid sequence(s) into a subject may be direct, by directly exposing the subject to the nucleic acid(s) or nucleic acid(s)-carrying vector, or indirect, wherein cells are transformed with the nucleic acid(s) in vitro, then transplanted into the subject: termed in vivo and ex vivo gene therapy respectively. In specific embodiments, a nucleic acid sequence(s) of/in this disclosure is incorporated into a nucleic acid expression vector(s) which is administered to the subject, and/or to a cell(s) to be administered to the subject, wherein one or more of the following scenarios are contemplated: nucleic acid sequence or vector thereof is introduced into one or more cells by direct injection of naked DNA or by use of microparticle bombardment (e.g., a gene gun; Biolistic, Dupont; Sanford et al., U.S. Pat. No. 4,945,050), expression vector(s) is a defective/attenuated retroviral or other viral vector (e.g. see U.S. Pat. No. 4,980,286), administration is by way of a matrix with in situ scaffolding in which the nucleic acid sequence(s) and/or vector(s) thereof is contained (e.g., European Patent No. EP0741785BI and U.S. Pat. No. 5,962,427), optionally wherein the nucleic acid sequence(s) and/or vector(s) thereof is, before administration, coated with lipids or cell-surface receptors or transfecting agents, encapsulated in liposomes, microparticles (Tice et al., U.S. Pat. No. 4,542,025), or microcapsules, or administered in linkage to a peptide(s) which is known to enter the cell/nucleus e.g. in linkage to a ligand(s) subject to receptor-mediated endocytosis (see, e.g., Wu and Wu, 1987, J. Biol. Chem. 262:4429-4432) (which can be used to target cell types specifically expressing the receptors). In some embodiments, nucleic acid-ligand complexes can be formed in which the ligand comprises a fusogenic viral peptide to disrupt endosomes, allowing the nucleic acid sequence to avoid lysosomal degradation. In yet another embodiment, the nucleic acid(s) can be targeted in vivo for cell specific uptake and expression, by targeting a specific receptor (see e.g. PCT Publication Nos: WO92/06180, WO92/22635, WO92/20316, WO93/14188, WO93/20221). Alternatively, the nucleic acid(s) can be introduced intracellularly and incorporated within host cell DNA for expression, by homologous recombination (Koller and Smithies, 1989, Proc. Nat). Acad. Sci. USA 86:8932-8935; and Zijlstra et al., 1989, Nature 342:435-438). In specific embodiments, at least one viral vector(s) that contains at least one nucleic acid sequence(s) of/in this disclosure is used. For example, a retroviral vector(s) can be used (see e.g. Miller et al., 1993, Meth. Enzymol. 217:581 599). These retroviral vectors contain the components necessary for the correct packaging of the viral genome and integration into the host cell DNA. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114. Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the Central Nervous System (CNS), endothelial cells, and muscle. Adenoviruses have the advantage of being capable of infecting non-dividing cells. Kozarsky and Wilson, 1993, Current Opinion in Genetics and Development 3:499-503 present a review of adenovirus based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234, PCT Publication WO94/12649; and Wang et al., 1995, Gene Therapy 2:775-783. Adeno-associated virus (AAV) is another option for use in gene therapy (Walsh et al., 1993, Proc. Soc. Exp. Biol. Med. 204:289-300; and U.S. Pat. No. 5,436,146). Another option is to use an ancestral AAV such as Anc80 (Zinn et al. 2015, Cell Reports, 12(6): 1056-68). A viral vector(s) can be provided as a pharmaceutical composition comprising at least one appropriate pharmaceutically acceptable carrier and/or excipient, with an appropriate dose for the appropriate route of administration and regime selected. Another approach to gene therapy involves transferring a nucleotide sequence(s)/gene(s) to cells (optionally sourced from the subject, optionally stem cells from the subject, e.g. refer US20140234275A1) in vitro, e.g. in tissue culture, by one or more such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred nucleotide sequence(s)/gene(s). Those cells are then delivered/returned to a subject. In an embodiment, the nucleic acid sequence(s) is introduced into a cell prior to administration in vivo of the resulting recombinant cell. Such introduction can be carried out by any method known in the art, including but not limited to transfection, electroporation, microinjection, infection with a viral or bacteriophage vector(s) containing the nucleic acid sequence(s), cell fusion, chromosome-mediated gene transfer, microcellmediated gene transfer, spheroplast 1372718-2 74 fusion, etc. Numerous techniques are known in the art for the introduction of foreign nucleotide sequences/genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92(1985)) and any one or more of which may be used in accordance with the present disclosure. Preferably, in some embodiments, the used technique(s) should provide for the temporary or stable transfer of the nucleic acid sequence(s) to the cell, so that the nucleic acid sequence(s) is expressible by the cell and, optionally, heritable and expressible by its cell progeny. The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art. Cells into which a nucleic acid sequence(s) can be introduced for purposes of gene therapy encompass any desired, available cell type, and include but are not limited to epithelial cells, endothelial cells, keratinocytes, fibroblasts, muscle cells, hepatocytes, blood cells such as T lymphocytes, B lymphocytes, natural killer (NK) cells, monocytes, macrophages, neutrophils, eosinophils, megakaryocytes, granulocytes, various stem or progenitor cells, in particular hematopoietic stem or progenitor cells, e.g., as obtained from bone marrow, umbilical cord blood, peripheral blood, fetel liver, etc. In a preferred embodiment, the cell used for gene therapy is autologous to the subject. In specific embodiments, stem or progenitor cells are used. Any stem and/or progenitor cells which can be isolated and maintained in vitro can potentially be used in accordance with this embodiment of the present disclosure (see e.g. PCT Publication No. WO94/08598; Stemple and Anderson, 1992, Cell 7 1:973-985; Rheinwald, 1980, Meth. Cell Bio. 21A: 229; and Pittelkow and Scott, 1986, Mayo Clinic Proc. 61:771).

At least one recombinant adeno-associated virus (AAV; e.g.: AAV1, AAV2, AAV4, AAV5, AAV8, AAV9, AAV-PHP.B, or AAVrh10; US20190358346A1, U.S. Pat. No. 8,865,881B2, WO2012142529A2, RU2020125073A, [237], patent applications/patents/papers that one or more of these cite, and/or that cite one or more of them), and/or self-complementary adeno-associated virus (scAAV), and/or other gene therapy vector(s) of the art (somatic and/or germline), optionally one invented/discovered by James Wilson and/or co-worker(s)/student(s)/affiliate(s)/compan (y/ies) (e.g. Regenxbio, Dimension Therapeutics, Passage Bio etc.), optionally/preferably one that has been used in a clinical trial(s), optionally a clinical trial listed on the clinicaltrials.gov website, preferably wherein it has passed Phase I clinical trials, for use as a medicament/therapeutic/cosmetic in a subject, optionally co-administered (before and/or concurrent and/or after) with an immunosuppressant/anti-inflammatory compound(s)/method(s)/prednisone, optionally wherein the AAV(s) and/or vector(s) persists for a period of time after administration, which can be permanent, as an episome(s)/extra-chromosomal genetic material in one or more cells of the subject, wherein any AAV subtype/serotype, including any modified/engineered/hybrid/pseudotyped derivative(s), is contemplated by, and componentry to, this disclosure, illustratively AAV2 and/or AAV8 and/or AAV9 [which can be administered, to illustrate, intravenously and can cross the blood-brain barrier in mice [238]], wherein AAV9 is used in Zolgensma, an FDA approved gene therapy), optionally wherein the vector is single stranded or a monomeric duplex vector, optionally wherein the AAV serotype(s) has greater affinity for a cell type(s) and/or cells of the nervous system, wherein the gene therapy vector comprises AAV capsid proteins (optionally of AAV serotype 5) and at least one nucleotide sequence(s) encoding at least one IF1 protein/fragment (or sequence variant thereof), and/or fusion protein(s) thereof, optionally at least one sequence of SEQ ID NO: X (or a fragment thereof, or concatenated fragments thereof, wherein X can be 1, or the number of sequences in the Sequence Listing component to this application, or any integer between 1 and the total number of sequences in the Sequence Listing component to this application), flanked by AAV Inverted Terminal Repeats (ITRs, optionally AAV2 ITRs), wherein the nucleotide sequence(s) of interest is operably linked to expression control element(s) comprising a promoter (and optionally a terminator sequence and/or a Woodchuck Hepatitis Virus [WHP] Posttranscriptional Regulatory Element [WPRE]) that produces expression in a cell(s), preferably an effective amount, of at least one peptide/protein/amino acid sequence(s) of/in this disclosure, preferably to obtain a therapeutic/aesthetic/desirable/cosmetic effect in the subject, wherein the promoter can be selected (without restriction) from the group consisting of. cytomegalovirus (CMV) promoter, phosphoglycerate kinase (PGK), b-globin, CBA, SV40, elFalpha, CAG promoter (a combination of the cytomegalovirus early enhancer element and chicken beta-actin promoter), artificial composite promotor (e.g. comprising CMV enhancer, chicken β-actin promoter, and MVM intron [PCBh]), glial fibrilary acidic protein (GFAP) promoter, synapsin-1 promoter, Neuron Specific Enolase (NSE), tissue specific promoters (e.g. Central Nervous System specific, e.g. neuron and/or glia specific), and inducible promoters such as a Tet-operon derived promoter, optionally wherein the gene therapy vector(s) is administered to the brain (one or more regions thereof e.g. (non-limiting) basal ganglia e.g. substantia nigra)/cerebrospinal fluid, optionally by lumber injection and/or injection into the cisterna magna and/or the eye (non-limiting e.g. intraretinal). A pharmaceutical composition comprising at least one AAV(s) described herein and at least one pharmaceutically acceptable excipient/carrier, optionally wherein the dose of the virus is in a range between 109 to 1020 genome copies (CG) per composition, administered systemically or locally, optionally wherein the administration route(s) circumnavigates the blood-brain barrier, optionally via the nasal route, and/or by intraventricular, intracerebral, intracisternal, intracerebroventricular, and/or intrathecal direct injection, among others. A plasmid(s) comprising the sequence of a recombinant AAV vector: an expression cassette flanked on either side by the ITRs of an AAV, wherein said expression cassette comprises a promoter, a coding region for at least one polynucleotide(s) of/in this disclosure, and optionally a terminator sequence. In other embodiments the viral vector(s) can be derived from adenovirus, retrovirus, lentivirus, herpes virus or other. Use of a vector(s) as herein defined for the manufacture of a medicament/pharmaceutical composition, optionally for the treatment/amelioration/prevention/reversal/combat of one or more diseases or disorders or physiological processes (and/or one or more of its consequences) or an unwanted/undesirable aesthetic(s) specified herein, for example, by a method comprising administering an effective amount of the vector(s), and/or pharmaceutical/cosmetic composition(s)/medicament thereof, to a subject in order to treat/improve/enhance the subject. Use of a vector(s) as herein defined, carrying at least one nucleotide sequence(s) encoding at least one IF1 protein/fragment (and/or sequence variant thereof), and/or fusion protein(s) thereof, in use as an oncolytic/anti-cancer virus is componentry to this disclosure, optionally wherein it is administered locally, rather than systemically, to the subject. Use of a vector(s) as herein defined, optionally AAV2, carrying at least one nucleotide sequence(s) encoding at least one IF1 protein/fragment (and/or sequence variant thereof), and/or fusion protein(s) thereof, to treat age-related macular degeneration (AMD) in a subject, optionally via local administration of the vector(s), and/or medicament/pharmaceutical composition thereof, to one or both eyes of the subject, optionally via intravitreal and/or sub-retinal administration. Dual, overlapping vector strategies (splitting the coding sequence over two or more vectors [237]) are componentry to this disclosure.

Liposomes, fusogenic liposomes, exosomes, nanocarriers, nanomotors, nanoparticle-stabilized nanocapsules (NPSCs), lipid preparations and lipid nanoparticles (LNPs) can also be used as vectors [239-241] in a composition/method of this disclosure. A variety of these types of vectors are known in the art (see, for example: U.S. Pat. No. 5,399,346; Bandara et al., DNA and Cell Biology, 11:227-231(1992); Berkner, Biotechniques 6:616-629(1989); U.S. Pat. No. 5,240,846 to Collins et al., issued Aug. 31, 1993; Culver and Blaese, TIG 5:171-178(1994); Goldman et al, Gene Therapy 3:811-818(1996); Hamada et al., Gynecologic Oncology 63:219-227(1996); Holmberg et al., J. Liposome Res. 1:393-406(1990); Hurford et al., Nature Genetics 10:430-435(1995); Karlsson et al., The EMBO J. 5:2377 2385(1986); Kleinerman et al., Cancer Res. 55:2831-2836(1995); Krul et al., Cancer Immunol. Immunother. 43:44-48(1996); U.S. Pat. No. 5,532,220 to Lee et al., issued Jul. 2, 1996; Liu et al., Nature Biotechnology 15:167-173 (1997); Mathiowitz et al., Nature 386:410- (1997); Nabel et al. Proc. Natl. Acad. Sci. USA 90:11307-11311(1993); Nabel et al., Science, 14 September: 1285-1288(1990); Ram et al., Cancer Res. 53:83-88(1993); Rosenfeld et al., Cell 68:143-155(1992); U.S. Pat. No. 5,580,859 to Felgner et al., issued Dec. 30, 1997 WO 98/13353 to Whitney et al., published Apr. 2, 1998; U.S. Pat. No. 5,298,429 to Evans et al., issued Mar. 29, 1994; U.S. Pat. No. 5,514,561 to Quante et al., issued May 7, 1996; WO96/24301 to The University of Edinburgh, published Oct. 27, 1994; WO96/30540 to The Regents of the University of California, published Oct. 3, 1996; Larrick and Burck, Gene Therapy, Application of Molecular Biology, Elsevier, NY (1991); and Pinkert, Transgenic Animal Technology, a Laboratory Handbook, Academic Press, Inc., San Diego.

A gene therapy construct can also be a naked DNA construct such as a plasmid(s), which is useful in a gene therapy treatment system (see, for example, U.S. Pat. No. 5,580,859 to Felgner et al., issued Dec. 3, 1996; U.S. Pat. No. 5,703,055 to Felgner et al., issued Dec. 30, 1997; U.S. Pat. No. 5,846,946 to Huebner et al., issued Dec. 8, 1998; and U.S. Pat. No. 5,910,488 to Nabel et al., issued Jun. 8, 1999), wherein this option is herein contemplated.

mRNA(s) and LNP(s)

Combination of at least one mRNA sequence of/in this disclosure (optionally incorporating one or more, independently selected, non-typical nucleosides e.g. pseudouridine or 1-methyl pseudouridine instead of uridine, and/or methyl-cytosine instead of cytosine, at one or more mRNA sequence positions) and one or more lipid nanoparticles (LNPs), optionally wherein the LNPs used are as disclosed in a patent application/patent filed by (and/or assigned to) one or more of Moderna Therapeutics Inc. (e.g. refer https://patents.google.com/?assignee=Moderna+Therapeutics), Arbutus Biopharma Corporation, Acuitas Therapeutics, Cure Vac, BioNTech, Alnylam Pharmaceuticals Inc., Arrowhead Pharmaceuticals or similar company, optionally one or more of U.S. Pat. Nos. 9,404,127, 9,364,435, 8,058,069 (and/or a reference(s) therein), optionally as used in a clinically utilized coronavirus vaccine e.g. that developed by Moderna Therapeutics Inc. for COVID-19 (mRNA-1273), e.g. as disclosed in one or more of U.S. Pat. Nos. 10,703,789, 10,702,600, 10,577,403, 10,442,756, 10,266,485, 10,064,959, 9,868,692. Some background reading: Moss et al. (2019) “Lipid Nanoparticles for Delivery of Therapeutic RNA Oligonucleotides” Mol. Pharmaceutics 16(6): 2265-2277, Kowalski et al. (2019) “Delivering the messenger: advances in technologies for therapeutic mRNA delivery”. Molecular Therapy. 27(4): 710-728, and references therein.

“Tet-Off” and “Tet-On” Inducible Gene Expression Embodiments of this Disclosure

In a “Tet-Off′ system, gene expression is under the control of a tetracycline-responsive promoter element (TRE), which permits gene expression if the tetracycline-controlled transactivator protein (tTA) binds, wherein tTA binding is blocked by the presence of tetracycline or doxycycline or similar compound structure. In a “Tet-On” system, gene expression is under the control of a tetracycline-responsive promoter element (TRE), which permits gene expression if the reverse tetracycline-controlled transactivator (rtTA) binds, wherein rtTA binding is enabled and conditional upon the presence of tetracycline or doxycycline or similar compound structure. So, one can transfer a gene into an organism under the control of a TRE promoter, and then control this gene's expression, spatially and/or temporally, by (1) controlling whether tTA or rtTA is expressed (which is set by which of these genes is also gene transferred into the organism) and (2) whether a tetracycline type compound structure is present or absent (set by compound administration or lack thereof). For example, generated transgenic mice with a transgenic gene encoding a mutant human IF1 protein (with a histidine in its “pH dependence motif” {refer FIG. 10} substituted with lysine) under the control of a TRE promotor. Furthermore, these mice had another exogenous gene present, for tTA protein expression, but which was only expressed in its forebrain neurons, and thence the mutant human IF1 gene was only expressed in forebrain neurons (wherein this expression is dependent on absence of tetracycline or similar structure). doesn't specify the ambient temperature these mice were housed at. With this omission, I presume normal room temperature, which is typically in the range 20-25° C. is an illustrative example of how an IF1 gene copy, or a mutant thereof, from the same or different species, can be transferred into an organism in order to increase its IF1 protein expression. This example shows it is safe in mouse brain (more specifically neurons in forebrain [131], is incorrect to refer to whole brain as it does) to increase IF1 protein content by three times, wherein the delta increase in [IF1 protein] occurs with a mutant human IF1 protein form with increased inhibitory potency against F₁F₀ ATP hydrolysis at pH 8, which in reduces F₁F₀ ATP hydrolysis capability by ˜35%, which demonstrates the safety of inhibiting F₁F₀ ATP hydrolysis in vivo, at least specifically in forebrain neurons (mice were “normal in appearance, home-cage behavior, reproduction, and longevity up to 1-year follow-up”). For more data and analysis on this herein, refer FIG. 6 and legend, and then FIGS. 7 and 8 and their legends. In alternative embodiments, tTA is expressed in all the tissues of the mouse, not just the brain (or part(s) thereof), and this mouse, and others like it, are entered into a lifespan study. These mice will have a longer health- and/or life-span than control mice (control mice optionally have the transgene for tTA but not the transgene for mutant IF1 mutant protein, or are the same genetically but are administered doxycycline, or other tetracycline, so that they don't express the extra, mutant IF1 protein gene), especially if the mice are kept at 37° C.

Further “Tet-Off” Embodiments of this Disclosure

A disclosure embodiment is for at least one transgenic IF1 protein/fragment (or sequence variant thereof) coding nucleotide sequence (herein often referred to as an “IF1 gene” for shorthand), optionally with a nucleotide change(s) that causes at least one amino acid change at the “phosphorylation control switch” residue and/or the “pH dependence motif” in the IF1 protein coded for (these IF1 protein elements are defined in FIG. 10), to be expressed only, or only substantially, or disproportionally, or more, in one or more of a specific cell type/tissue/organ/area of a subject, optionally wherein this selective IF1 protein/fragment (or sequence variant thereof) expression reduces F₁F₀ ATP hydrolysis and slows the metabolic/aging rate in this sub-section(s) of the subject, wherein this sub-section(s) generates less metabolic heat, but this is substituted for by heat transfer from surrounding body areas. For illustrative example, by making a transgenic mouse that expresses TA specifically in a certain mouse body tissue, by putting tTA expression under the control of a tissue specific promoter, wherein this tissue specific expression of tTA then drives tissue specific expression of at least one introduced IF1 gene (encoding at least one IF1 protein/fragment or sequence variant thereof) whose expression is under the control of a tetracycline-responsive promoter element (TRE). For non-limiting example, putting tTA expression under the control of a dopamine neuron specific promoter such as that for tyrosine hydroxylase (the first and rate-limiting enzyme for dopamine synthesis) [242], or the dopamine transporter (DAT, required for dopamine re-uptake into dopaminergic neurons) [243-244], or Pitx3 (transcription factor involved in dopaminergic neuron differentiation), or D1A dopamine receptor subtype, which then drives specific expression of at least one IF1 gene (encoding at least one IF1 protein/fragment or sequence variant thereof) in dopaminergic neurons, wherein this IF1 gene(s) is under the control of a tetracycline-responsive promoter element (TRE). Greater IF1 protein expression in dopaminergic neurons reduces their F₁F₀ ATP hydrolysis, especially if the extra IF1 protein has an amino acid residue that cannot be phosphorylated (optionally alanine) at its “phosphorylation control switch” position and/or a H49K (or H49A or H49R) substitution (position 49 using “mature” {Mitochondrial Import Sequence [MIS] cleaved off} human IF1 protein numbering) in its “pH dependence motif”, which slows the metabolic/aging rate in dopaminergic neurons, which reduces the subject's risk/progression of Parkinson's disease, wherein these dopaminergic neurons generate less metabolic heat, but this is substituted for by heat transfer from surrounding body areas, such that the dopaminergic neurons are maintained at normal body temperature.

A useful control is that this subject, when administered (e.g. in drinking water) with tetracycline, has the same risk/progression of Parkinson's disease as a control subject without these genetic manipulations (which are, to recap, addition of tTA gene, under dopaminergic neuron specific promoter, and addition of at least one IF1 gene, encoding at least one IF1 protein/fragment or sequence variant thereof, under the control of a tetracycline-responsive promoter element, TRE). In a different embodiment, tTA expression is put under the control of a photoreceptor specific promoter, such as that for rhodopsin (or other opsin), which then drives specific expression of at least one introduced IF1 gene, encoding at least one IF1 protein/fragment or sequence variant thereof, in photoreceptors, wherein this IF1 gene(s) is under the control of a tetracycline-responsive promoter element (TRE). Greater IF1 protein expression in photoreceptors reduces their F₁F₀ ATP hydrolysis, especially if the extra IF1 protein has an amino acid residue that cannot be phosphorylated (optionally alanine) at its “phosphorylation control switch” position and/or a H49K (or H49A or H49R) substitution (position 49 using “mature” {after MIS cleaved off} human IF1 protein numbering) in its “pH dependence motif”, which slows the metabolic/aging rate in photoreceptors, which reduces the subject's risk/progression of aging related/correlated eye disease(s), optionally Age-related Macular Degeneration (AMD), wherein these photoreceptors generate less metabolic heat, but this is substituted for by heat transfer from surrounding body areas, such that the photoreceptors are maintained at normal body temperature.

In a disclosure embodiment, the experiment of is repeated but with the mutant IF1 protein transgene expressed in a different part(s) of the mouse. So, instead of using B16-Tg(Camk2a-tTa)1Mmay/J mice, which express tTA in forebrain neurons, as used, a different transgenic mouse type can be used, which expresses tTA in a different part(s) of the mouse body, which drives extra/mutant IF1 protein expression in that different part of the mouse body. Illustratively, a variety of transgenic mice that express tTA in different body areas/tissues/organs are available to those of the art, e.g. refer to a database of such mice at [245], and a number are commercially available, for illustrative example from The Jackson Laboratory (USA). Wherein (stock numbers given are for The Jackson Laboratory), for non-limiting example, B6.Cg-Tg(GFAP-tTA)110Pop/J mouse (stock No: 005964) has tTA expression driven by the human glial fibrillary acidic protein (GFAP) promoter and expresses tTA in astrocytes, B6.Cg-Tg(Sirpa-(TA)AUmri/J mouse (stock No: 023970) has tTA expression driven by mouse signal-regulatory protein alpha (Sirpa) promoter and expresses tTA in dentate granule cells of the hippocampus, B6.Cg-Tg(Scg2-(TA)1Jt/J mouse (stock No: 008284) has tTA expression driven by mouse secretogranin II promoter and expresses tTA in the brain, especially the suprachiasmatic nucleus, B6.Cg-Tg(Eno2tTA)5030Nes/J mouse (stock No: 003763) has tTA expression driven by rat neuron-specific enolase (Eno2) promoter and expresses tTA at high levels in the striatum and to a lesser extent in the cerebral cortex and hippocampus, B6;C3-Tg(NEFH-tTA)8Vle/J mouse (stock No: 025397) has tTA expression driven by human neurofilament heavy polypeptide (NEFH) promoter and expresses tTA in neurons and large-caliber axons of the brain and spinal cord, B6;129S-Slc6a3tm4.1(tTA)Xz/J mouse (stock No: 027178) has tTA expression driven by mouse Slc6a3 (solute carrier family 6, neurotransmitter transporter, dopamine, DAT) promoter and expresses tTA in dopamine neurons, NOD.Cg-Tg(Ins2-tTA)1Doi/DoiJ mouse (stock No: 004937) has tTA expression driven by rat insulin promoter (Ins2, commonly designated RIP) and expresses tTA in pancreatic beta cells, FVB.Cg-Tg(Myh6-tTA)6Smbf/J mouse (stock No: 003170) has tTA driven by rat alpha myosin heavy chain promoter and expresses tTA in cardiac myocytes. Many other such mice are commercially available, and/or are known to those of the art (e.g. from the literature) and other such mice, with tTA (and/or rtTA) specifically expressed in other cell type(s)/tissue(s)/organ(s), can be generated using techniques of the art, wherein how to apply these techniques to other mammal species is known to those of the art.

A disclosure embodiment is for at least one IF1 gene, encoding at least one IF1 protein/fragment or sequence variant thereof, optionally with a nucleotide change(s) that causes an amino acid residue change(s) at the “phosphorylation control switch” and/or the “pH dependence motif” (these IF1 elements are defined in FIG. 10) in the IF1 protein/fragment coded for, to be expressed ubiquitously in a subject, in every tissue. For illustrative example, by making a transgenic mouse that expresses tTA ubiquitously, by putting tTA expression under the control of a ubiquitous (e.g. promoter for actin gene) or synthetic (e.g. CAG promoter [246]) promoter, wherein this ubiquitous expression of tTA can then drive ubiquitous expression of an introduced IF1 gene(s), encoding at least one IF1 protein/fragment or sequence variant thereof, whose expression is under the control of a tetracycline-responsive promoter element (TRE).

More Direct IF1 Genetic Manipulations of this Disclosure

Some embodiments are to render localized or ubiquitous expression of at least one extra IF1 gene(s), encoding at least one IF1 protein/fragment or sequence variant thereof, in a subject without using TRE and tTA or rtTA. So, to illustrate, and not restrict, to render tissue-specific (extra) IF1 protein/fragment (or sequence variant thereof) expression in a subject by administering at least one IF1 gene(s), encoding at least one IF1 protein/fragment or sequence variant thereof, locally (rather than systemically) and/or under the control of a tissue specific promoter or, alternatively, rendering ubiquitous (extra) IF1 protein/fragment (or sequence variant thereof) expression in a subject by introducing at least one IF1 gene(s), encoding at least one IF1 protein/fragment or sequence variant thereof, under the control of a ubiquitous promoter, optionally a synthetic promoter. Without restriction, there are two possible options: (1) the introduced IF1 gene(s), encoding at least one IF1 protein/fragment or sequence variant thereof, under the control of an introduced promoter (and/or enhancer and/or other regulatory element[s]), is introduced into the subject, or (2) the IF1 gene(s) only, encoding at least one IF1 protein/fragment or sequence variant thereof, is introduced into the subject, wherein it inserts into the genome at a place that renders its expression under the control of a cell/tissue/organ/cell-type specific or ubiquitous promoter already in the genome. The IF1 gene(s), encoding at least one IF1 protein/fragment or sequence variant thereof, can be introduced into one or more cells of the subject, somatic and/or germline cell(s) thereof, in vivo or ex vivo (the latter wherein cell(s) are removed from the subject, and at least one IF1 gene, encoding at least one IF1 protein/fragment or sequence variant thereof, is introduced into the cell(s), and the cell(s) are returned back into the subject afterwards), wherein optionally the introduced nucleotide sequence(s), which includes at least one IF1 gene(s) encoding at least one IF1 protein/fragment or sequence variant thereof, is introduced as naked nucleotide material (optionally, without restriction, by one or more of heat shock, cell squeezing, using calcium phosphate to bind the DNA, electroporation, gene gun, sonoporation, photoporation, magnetofection, magnet assisted transfection, lipofection, impalefection, optical transfection, nucleofection, protofection, hydroporation, hydrodynamic delivery, microinjection {DNA is injected through the cell's nuclear envelope directly into the nucleus}, pronuclear injection {after the sperm enters the egg, but before the genetic material of the sperm and egg fuse, genetic material is injected into the pronucleus of either the sperm or egg, when these pronuclei become visible, which is the first sign of successful fertilization; this oocyte is then implanted in the oviduct of a pseudopregnant female [e.g. induced when female is bred by an infertile male], the offspring thence carry the genetic modification}, embryonic Stem Cell-Mediated Gene Transfer {gene is transfected into an embryonic stem cell(s) that is then inserted into a subject blastocyst(s), which is then implanted into foster mother, resulting offspring is/are chimeric, further mating can produce mice fully transgenic subject(s) with the gene of interest}) or in a vector(s), wherein the vector(s) can be, without restriction, one or more of a plasmid, episome, lipoplex (optionally with cationic lipids), liposome, cationic liposome, liposome coated in a polymer called polyethylene glycol, polymersome (synthetic liposome, made of amphiphilic block copolymers; copolymer is a polymer derived from more than one species of monomer), lipid nanoparticle (LNP, non-limiting e.g. as used for an FDA/EMA approved RNA/RNAi therapy), non-liposomal lipid formulation, polyplex (complex of polymer with DNA), polymer, polyamine, polyethylenimine, dendrimer (highly branched macromolecule with a spherical shape), inorganic nanoparticle (e.g. gold, silica, iron oxide, calcium phosphate etc.), organic nanoparticle, fugene, Fugene 6, Cell Penetrating Peptide (CPP), virosome (viral envelope {phospholipid membrane and surface glycoproteins} of a virus that, instead of viral genetic material, contains at least one therapeutic agent of/in this disclosure e.g. at least one nucleotide sequence of/in this disclosure), virus/recombinant virus (replication-competent, or replication-defective/attenuated {coding region(s) for gene(s) necessary for virion replication/packaging is replaced with other gene(s), corrupted or deleted}), retrovirus (has a single stranded RNA genome that codes for a reverse transcriptase enzyme that produces a double stranded DNA sequence that is inserted into host genome by viral enzyme integrase; most retroviruses can only insert into actively dividing cells, which makes them appropriate for delivering at least one IF1 gene(s), encoding at least one IF1 protein/fragment or sequence variant thereof, for anti-cancer therapy; however, they tend to insert into the genome unpredictably, so “insertional mutagenesis” is a concern, however, in an embodiment, sequence(s) is incorporated that directs a retroviral coded DNA sequence(s) to be inserted at a specific locus or loci in the genome e.g. utilizing the beta-globin locus control region and/or by inserting a sequence for a zinc finger nuclease which cuts the genome at a place that the viral DNA is desired to be inserted; retroviral vector particles with tropism for various target cells have been designed in the art), lentivirus (a genus of retrovirus; can insert into non-dividing cells; HIV is an example; enhancers can be used to improve transduction efficiency such as, without limitation, polybrene, protamine sulfate, retronectin, and DEAE Dextran), gammaretrovirus (a genus of retrovirus; non-limiting e.g. moloney retrovirus), adenovirus (doesn't integrate into genome, exists as an episome/extra-chromasomal DNA, not replicated during cell division, so is diluted by cell divisions and so is better used for non-dividing cells), adeno-associated virus (AAV; single stranded DNA virus {becomes double stranded in the cell}; can be integrated into genome, mostly at a known site in humans {designated AAVS1, in human chromosome 19}, but is more likely to remain episomal {replicating without incorporation into the chromosome}; in use as gene therapy vector the low integrative probability can be reduced further by removing its integrative capacity by removal of the rep and cap open reading frame sequences (each containing a number of overlapping genes) from the DNA of the vector, wherein these are replaced with the desired gene(s), together with a promoter to drive transcription of the gene(s) (and optionally a terminator sequence), which is inserted between the inverted terminal repeats (ITR); useful for dividing and non-dividing cells; doesn't cause any disease that we know of; can't replicate without a helper virus (e.g. adenovirus, thence its adeno-associated virus name) but recombinant AAV forms are available that can replicate without a helper virus; AAV has been used as gene therapy vector in clinical trials e.g. for retinal gene therapy by subretinal and/or intravitreal injection {e.g. Voretigene neparvovec (Luxturna)}, wherein a disclosure embodiment is to deliver at least one IF1 gene(s), encoding at least one IF1 protein/fragment or sequence variant thereof, similarly to treat/ameliorate/prevent/combat an aging-related/linked/correlated eye(s) disease/condition, optionally Age-related Macular Degeneration, AMD; all AAV serotypes are [contemplated by/componentry to] the present disclosure (AAV2 is the most well studied in humans; most AAV serotypes show neuronal tropism (amongst other tropisms), while AAV5 also transduces astrocytes) as are hybrid AAVs (e.g. genome components of one AAV, capsid of another e.g. AAV2/5, e.g. hybrid capsid from different strains, which can broaden tropism e.g. AAV-DJ has hybrid capsid of 8 different strains and so has broader tropism than ony one of these strains) and pseudotyped AAVs e.g. pseudotyped to modify their tropism [the cell type(s) that they infect] and/or to reduce immune reaction), self-complementary adeno-associated virus (scAAV; viral vector engineered from the naturally occurring adeno-associated virus (AAV); double stranded DNA virus), pseudotyped viruses (in which the endogenous viral envelope proteins have been replaced by either envelope proteins from other viruses, or by chimeric proteins, e.g. to alter the cell type(s) that the virus infects [tropism]; frequently used is glycoprotein G of vesicular stomatitis virus (VSV), short VSV-G, an envelope protein that transduces to all cell types), hybrid vector (=genetically engineered virus, to have desired vector characteristics e.g. to have qualities of more than one vector), replication-deficient Herpes simplex virus (human neurotropic virus, infects neurons, good for delivering gene(s) to nervous system), papilloma virus or other type of virus vector used in gene therapy systems or genetic manipulation of cells, or other vectors of the art, Cell Penetrating Peptide (e.g. protein transduction domain of Tat protein of HIV-1 virus) etc. This list is not exhaustive, other methods of introducing new genetic material to a subject are known to those of the art. Including using an ancestral or ancestral like AAV, such as Anc80 [247], which has significant merit because it doesn't stimulate immune reactions, because modern organisms haven't encountered this virus for generations. In some disclosure embodiments at least one IF1 gene, encoding at least one IF1 protein/fragment or sequence variant thereof, is introduced into the mitochondrial genome (protofection). In some disclosure embodiments, a composition/method/route herein described for use with at least one IF1 gene, encoding at least one IF1 protein/fragment or sequence variant thereof, is used with corresponding mRNA instead or in addition. Componentry to this disclosure is a combination of vectors, for example, to illustrate and not restrict, a virus vector inside of a liposome or a plasmid vector inside of a lipid nanoparticle. Naked DNA/RNA sequence(s) of/in this disclosure, and/or vector(s) thereof, may be complexed or covalently or noncovalently conjugated or bound to other molecules such as, to illustrate and not restrict, cationic lipids, packaged within liposomes, incorporated into hydrogels, cyclodextrins, biodegradable nanocapsules, or bioadhesive microspheres. Small molecules such as folate may be conjugated to nucleic acid molecules to enhance transport across the blood-brain barrier (Wu, D. et al. (1999) Pharm. Res. 16:415-19.).

A gene therapy construct(s) may be delivered to a subject in cells. Cells containing gene therapy constructs may be derived from the subject and/or another subject of the same or different species. Gene therapy construct(s) may be introduced into these cells ex vivo by, to illustrate and not restrict, viral transfection, electroporation, membrane fusion with liposomes, high velocity bombardment with DNA coated microprojectiles, incubation with calcium-phosphate-DNA precipitate, transfection with DEAE-dextran, direct microinjection, or other method(s) known in the art. These cells are then delivered to the subject by any of a variety of means, including implantation or injection. The cells may express the gene therapy construct in vivo to obtain therapeutic effect(s) in the subject. Alternatively, or in addition, after introduction into the subject, the cells containing the gene therapy construct(s) may replicate and/or package the gene therapy construct(s) such that endogenous cells in the patient may be infected, transformed, or transfected with the gene therapy construct(s) and thereby express it. Cells containing gene therapy construct(s) may be enclosed in structures composed of polymers or other materials to retain them at the instillation site(s) or to protect them from the subject's cellular immunity mechanisms. To guide without limitation, for cells containing viral expression construct(s), about 105 to about 10⁸ cells may be delivered to an appropriate site of the subject, but wherein the skilled practitioner is able to determine the most appropriate dose.

### Gene Editing

Componentry to this disclosure is to use gene/genome editing with engineered nuclease(s) and/or CRISPR/Cas9 and/or CRISPR/Cpf1 and/or Zinc finger nuclease(s) and/or Zinc-finger nickase(s) and/or Transcription activator-like effector nuclease(s) (TALEN) and/or meganuclease(s) and/or megaTAL(s) and/or homing endonuclease(s) and/or restriction enzyme(s) and/or endonuclease and/or nuclease(s) and/or “gene targeting” (in particular “gene knock-in”, replacement strategy based on homologous recombination) and/or Recombinant AAV mediated genome engineering (rAAV) and/or Multiplex Automated Genomic Engineering (MAGE) and/or Cre-Lox system(s) and/or Flp-FRT system(s) and/or similar and/or other genetic editing/engineering technology to edit a native IF1 gene in a subject to become a different IF1 gene of/in this disclosure, optionally with a nucleotide change(s) that causes an amino acid residue change(s) at the “phosphorylation control switch” (optionally serine to alanine substitution) and/or the “pH dependence motif” (optionally a H49K [or H49A or H49R] substitution at position 49, using “mature” {MIS cleaved off} human IF1 protein numbering) in the IF1 protein coded for (these IF1 protein elements are defined in FIG. 10), and/or to insert at least one nucleotide sequence(s) of/in this disclosure, which is a nucleotide sequence(s) that codes for one or more of the IF1 protein/fragment (or sequence variant thereof) sequence embodiments of/in this disclosure, into a genome, optionally in one or more cells of a subject, optionally one or more of a somatic cell(s), germline cell(s), gamete(s) (e.g. sperm[s] or egg[s]), gametocyte(s), spermatocyte(s), oocyte(s), fertilized egg(s), pronuclear fertilized egg(s), embryonic stem cell(s), induced pluripotent stem cell (IPSC), or other(s). Componentry to this disclosure is to “knock-in”, wherein this term is well understood in the art (e.g. one of the art is very familiar with “knock-out” and “knock in” mice, and protocols used), an IF1 protein/fragment (or sequence variant thereof) coding nucleotide DNA sequence of/in this disclosure, optionally with a nucleotide change(s) that causes an amino acid residue change(s) at the “phosphorylation control switch” (optionally serine to alanine substitution) and/or the “pH dependence motif” (optionally a H49K [or H49A or H49R] substitution at position 49, using “mature” {MIS cleaved off} human IF1 protein numbering) in the IF1 protein coded for (these IF1 elements are defined in FIG. 10), in place of the native IF1 gene of the subject.

### Manipulating IF1 Gene Expression

An embodiment is to manipulate/change the promoter/enhancer/repressor/regulatory sequence(s) of an IF1 gene(s), to increase the expression of IF1 protein, in somatic and/or germline cell(s) of a subject, optionally wherein a new promoter/enhancer/regulatory sequence(s) is added/substituted in, optionally wherein this increased IF1 protein expression reduces F₁F₀ ATP hydrolysis and slows the metabolic/aging rate in this cell(s) of the subject, optionally wherein exogenous/ambient heat is administered to the subject to substitute for this lesser heat generation and/or greater body insulation (e.g. clothing) is administered to the subject to reduce heat loss per unit time, which correspondingly keeps the subject warm despite lesser heat generation.

Summary/Overview/Recap of IF1 Genetic Manipulations of this Disclosure

An embodiment of this disclosure is to change the sequence of a subject's IF1 gene, optionally conferring a greater inhibitory potency against F₁F₀ ATP hydrolysis at normal mitochondrial matrix pH 8, and/or increase the amount of IF1 protein in a cell(s) of a subject, optionally in all or the majority of cells in the subject or a subset of cell type(s) and/or cell(s) in certain tissue(s)/area(s) of the subject, optionally wherein change in the amount of IF1 protein is effected by (1) altering the regulatory sequence(s) of the subject's IF1 gene to effect greater IF1 gene expression, by greater transcription and/or translation, and/or (2) by adding another IF1 gene(s) to the subject's cell(s) genome, optionally wherein one or more of the added IF1 gene(s) are from a different (larger and/or longer living {greater maximal lifespan}) species and/or is a mutant IF1 gene, optionally producing an IF1 protein/fragment or sequence variant thereof with a greater inhibitory potency against F₁F₀ ATP hydrolysis at normal mitochondrial matrix pH 8, optionally with a nucleotide change(s) that causes an amino acid residue change(s) at the “phosphorylation control switch” (optionally serine to alanine substitution) and/or the “pH dependence motif” (optionally a H49K [or H49A or H49R] substitution at position 49, using “mature” {MIS cleaved off} human IF1 protein numbering) in the IF1 protein coded for (these IF1 protein elements are defined in FIG. 10), optionally, especially if many/all of the subject's cells have been manipulated in this way, greater exogenous/ambient heat is administered to the subject to substitute for lesser endogenous heat generation and/or greater body insulation (e.g. clothing) is administered to the subject to reduce heat loss per unit time, which correspondingly keeps the subject warm despite lesser metabolic rate/body heat generation.

### Example Embodiments: IF1 Gene Therapy for Parkinson's Disease

Firstly, gene therapy is now well established in the clinic, with thousands of clinical trials having taken place, with more being conducted, across dozens of countries for many indications, with notable approvals [248]. Secondly, for closer context, there are numerous reviews of gene therapy for Parkinson's disease in the literature e.g. [249-252]. A disclosure embodiment is to locally deliver, optionally bilaterally, optionally by injection into the subject's putamen (as used in [253]) and/or into the subject's basal ganglia/substantia nigra (SN)/substantia nigra pars compacta (SNpc), at least one IF1 gene(s), encoding at least one IF1 protein/fragment or sequence variant thereof, {optionally another gene(s) also, optionally one or more genes coding for an enzyme in dopamine synthesis e.g. for tyrosine hydroxylase, this enzyme catalyses the rate-limiting step of dopamine synthesis, e.g. for aromatic L-amino acid decarboxylase (AADC)}, optionally in a viral vector(s), optionally in a lentiviral viral vector(s) (optionally ProSavin [253]) and/or an adeno-associated viral vector(s) (optionally AAV2 [254]), to dopaminergic neurons, which reduces their F₁F₀ ATP hydrolysis, especially if the resulting extra IF1 protein/fragment has an amino acid residue that cannot be phosphorylated (optionally alanine) at its “phosphorylation control switch” position and/or a H49K (or H49A or H49R) substitution (position 49 using “mature” {MIS cleaved off} human IF1 protein numbering) in its “pH dependence motif”, which slows the metabolic/aging rate in dopaminergic neurons, which reduces the subject's risk/progression of Parkinson's disease, wherein these dopaminergic neurons generate less metabolic heat, but this is substituted for by heat transfer from surrounding body areas, such that the dopaminergic neurons are maintained at normal body temperature, optionally wherein this therapy is administered alongside one or more other therapies for Parkinson's disease known to those of the art e.g. L-DOPA administration, deep brain stimulation etc. In other embodiments, additionally or instead of, at least one IF1 gene(s) is administered to another brain region(s), optionally a brain region(s) (whose dysfunction is) implicated in Parkinson's disease e.g. optionally by infusion directly into the subject's subthalamic nucleus (as used in) [253].

### Example Embodiments: IF1 Gene Therapy for Osteoarthritis

### Example Embodiments: IF1 Gene Therapy Extends Mouse Lifespan

At least one IF1 gene(s), encoding at least one IF1 protein/fragment or sequence variant thereof, optionally with a nucleotide change(s) that causes an amino acid residue change(s) at the “phosphorylation control switch” and/or the “pH dependence motif” in the IF1 protein coded for (these IF1 protein elements are defined in FIG. 10), with a promoter that can drive its expression, optionally the CMV enhancer element and a chicken β-actin promoter containing its first intron (regulatory element used in [255]; alternatively IF1 gene(s) is flanked by β-actin promoter and terminator), is delivered to a nascent B6 (B6C3F1) mouse embryo by pronuclear microinjection, wherein the resulting mouse has this extra IF1 gene(s) (with regulatory element(s) for expression) in all its cells (checked by PCR for a number of different cells from different tissues isolated from the mouse, amount of IF1 protein/fragment (or sequence variant thereof) is also assayed, wherein the genetically modified mouse should have more IF1 protein/fragment (or sequence variant thereof) in its cells). This protocol is repeated to generate a number of such genetically modified mice and their lifespans, and/or those of their progeny as they are mated with each other and/or back-crossed with C57BL/6J mice one or more times, are monitored as compared to control mice (no genetic modification), wherein the genetically modified mice have longer health- and/or life-(median and maximal) span. Food intake and body weight is monitored for all mice over their lifespan. Mice are kept in specific pathogen free conditions. Optionally the study is repeated or run in parallel with mice of a different genetic background. More details on the protocol to follow (e.g. number of mice and statistics to be used, healthspan assays that can be used {e.g. cardiac pathology, cataract development, oxidative damage, mitochondrial deletions etc.} etc.) can be sourced from emulating the protocol of [255]. A protocol of a different study of genetic manipulation and lifespan in the literature can be used instead e.g. [256]. But crucially, distinctly, all mice in the present study are housed at 37° C. The genetically modified mice have more IF1 protein/fragment (or sequence variant thereof) in their cells, less F₁F₀ ATP hydrolysis per unit time, less oxidative phosphorylation per unit time, less heat generation per unit time (not detrimental when ambient temperature is 37° C.), less ROS production per unit time, less oxidative damage per unit time, slower aging and longer lifespan and/or healthspan.

Instead of the IF1 transgene(s) adding an extra IF1 gene copy (or copies) to the mouse genome, as above, in an alternative disclosure embodiment, the IF1 transgene replaces the native IF1 gene by a “genetic knock-in” (replacement strategy based on homologous recombination), optionally wherein the IF1 transgene has a nucleotide change(s) that causes an amino acid residue change(s) at the “phosphorylation control switch” and/or the “pH dependence motif” in the IF1 protein coded for (these IF1 protein elements are defined in FIG. 10). These mice also have slower aging and longer lifespan and/or healthspan. IF appropriate thermoregulatory countermeasures are taken, e.g. as aforementioned.

### Example Embodiments: IF1 Gene Therapy, Restricted to Brain, Extends “Brainspan”

Transgenic mice are made via pronuclear microinjection of mouse embryos with at least one IF1 transgene(s), optionally with a nucleotide change(s) that causes an amino acid residue change(s) at the “phosphorylation control switch” and/or the “pH dependence motif” in the IF1 protein coded for (these IF1 protein elements are defined in FIG. 10), under the control of human neurofilament heavy polypeptide (NEFH) promoter, or other brain/neuron/astrocyte specific promoter of the art, some (non-limiting) examples of which have been mentioned herein already, optionally using multiple IF1 gene copies with different promoters to ensure IF1 transgene expression in both neurons and astrocytes. The mice are housed at an ambient temperature between 21 and 37° C. These genetically modified mice have more IF1 protein in their brain, wherein there is less F₁F₀ ATP hydrolysis per unit time, less oxidative phosphorylation per unit time, less heat generation per unit time (not detrimental when ambient temperature is 37° C., and not detrimental when ambient temperature is lower because heat transfer from the rest of the body, especially via blood flow, maintains the brain at 37° C.), less ROS production per unit time, less oxidative damage per unit time, slower brain aging and longer “brainspan” i.e. less cognitive decline with age, lower risk/later onset/slower progression of age-related/correlated brain disease(s) e.g. Alzheimer's disease, dementia, Parkinson's disease etc.

Some Antibodies of this Disclosure

An antibody that can inhibit/reduce F₁F₀ ATP hydrolysis (for example inside a cell, preferably a eukaryote cell, or in a Sub-Mitochondrial Particle [SMP] assay of F₁F₀ ATP hydrolysis), and/or use thereof (for at least one use disclosed herein), is componentry to this disclosure. In some embodiments, such an antibody is (innovatively) acquired by raising antibodies against an IF1 protein or a fragment thereof (so not ATP synthase), especially a C-terminal IF1 protein fragment, e.g. from human IF1, e.g. bovine IF1-(44-84) fragment, or a sub-fragment thereof, e.g. ALKKHHENEISHHAK, e.g. HHENEISHH, e.g. HENEISH (fragments 69-83, 73-81, 74-80 of SEQ ID NO:660 respectively), or the equivalent section of the IF1 protein of a different species (e.g. by immunizing an animal [e.g. rabbit] with an IF1 protein, or a fragment thereof, and recovering/isolating/purifying antibodies specific for it from the animal's serum; “Antibodies. A Laboratory Manual” by Harlow E, Lane D (1988) Cold Spring Harbor, USA). Componentry to this disclosure is an antibody, and/or use thereof (for at least one use disclosed herein), raised against an IF1 protein, or fragment thereof, that can block/reduce IF1 tetramerization (and higher oligomerization), and/or that can inhibit/reduce F₁F₀ ATP hydrolysis, and doesn't significantly inhibit F₁F₀ ATP synthesis, in an SMP assay of the art (with IF1 protein present, at alkali pH).

An (optionally produced/isolated/purified/substantially purified/partially purified) antibody specific for, which specifically binds, IF1 protein and reduces F₁F₀ ATP hydrolysis (for example inside a cell, preferably a eukaryote cell, or in an SMP assay of F₁F₀ ATP hydrolysis) and/or use thereof (for at least one use disclosed herein). An (optionally produced/isolated/purified/substantially purified/partially purified) antibody specific for, which specifically binds, the C-terminal region of IF1 and reduces F₁F₀ ATP hydrolysis (for example inside a cell, preferably a eukaryote cell, or in an SMP assay of F₁F₀ ATP hydrolysis) and/or use thereof (for at one least one use disclosed herein).

At points in this disclosure that an antibody/antibodies is referred to, in some embodiments, one or more of the following is being referred to: a polyclonal, monoclonal, monospecific, chimeric (non-limiting e.g. mouse and human), chimeric monoclonal, humanized, humanized monoclonal, fully human, fully human monoclonal, antibody/antibodies. A hybridoma thereof is also contemplated. At all points that an “antibody” is referred to herein, and/or plural thereof, in alternative embodiments this is substituted with “intrabody” and optionally plural thereof.

These antibodies of this disclosure are some amino acid sequences of this disclosure.

### Some Methods of the Disclosure

A method of obtaining a compound(s) that reduces F₁F₀ ATP hydrolysis, optionally for at least one use disclosed herein, comprising the steps of:
i. generating a three-dimensional computer model of IF1 protein or fragment thereof, preferably existing as a dimer, optionally from an NMR or crystal structure thereof, optionally using the NMR structure of the C-terminal end (residues: 44-84) of bovine IF1 protein, which exists as a dimer [189];
ii. electronically (optionally using a program of the art such as, without limitation, Autodock, Autodock Vina, a Molecular Dynamics program(s), GOLD, Glide [Schrödinger, New York, NY, USA]) testing/screening a compound, or a library of compounds (each preferably smaller than 1000 Daltons, optionally a library of compounds wherein all or most adhere to Lipinski's rule of five, optionally a library of the art, optionally a “virtual library” {e.g. http://zinc.docking.org/; Shoichet, J. Chem. Inf. Model., 45(1): 177-82, 2005}, optionally a number of amino acid sequences), with defined spatial coordinates in order to identify a compound(s) that can bind H49 and/or surrounding residues, and/or H55 and/or surrounding residues (using “mature” [without MIS] bovine IF1 protein numbering); and
iii. optionally a compound(s) that can bind with affinity in (ii) is synthesized and experimentally tested to see if it can block/reduce IF1 protein tetramerization (and higher oligomerization), and/or to see if it can inhibit/reduce F₁F₀ ATP hydrolysis, and not significantly inhibit F₁F₀ ATP synthesis, in an SMP assay of the art (with IF1 protein present, at alkali pH).

A drug(s) (for at least one use disclosed herein) identified by the aforementioned method is componentry to the present disclosure. The use of a drug(s) (for at least one use disclosed herein) identified by the aforementioned method is componentry to the present disclosure.

Some Peptides of this Disclosure

In the following synthesis scheme, Compound 1 is available from Ambinter, Orléans, France, Compounds 3 and 6 from Fisher Scientific (part of Thermo Fisher Scientific). Using the teaching of this scheme, the equivalent change at the N and/or C-terminal ends of any amino acid(s), optionally proteinogenic, optionally histidine, or any amino acid sequence(s), preferably one or more of the amino acid sequences herein, optionally then used for one or more of the uses mentioned herein, is componentry to the present disclosure.

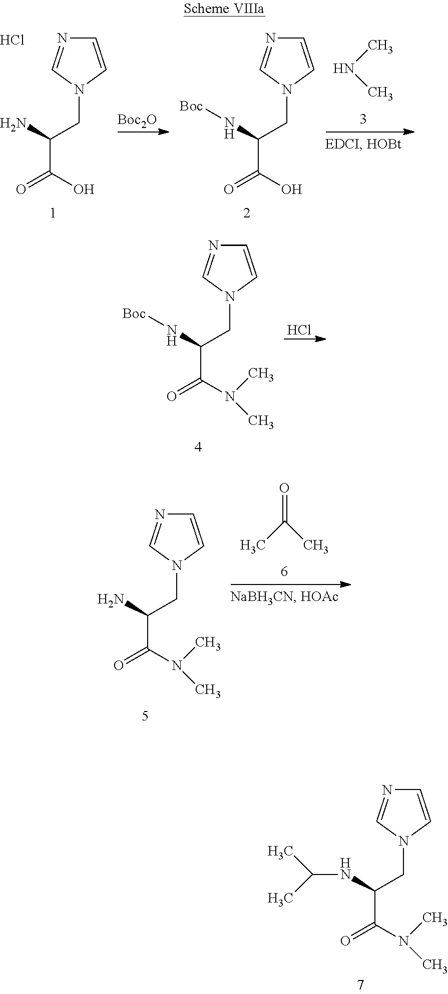

In the following scheme, how to produce the starting material (peptide synthesis, 2 histidines joined by a peptide bond) is clear to someone of the art.

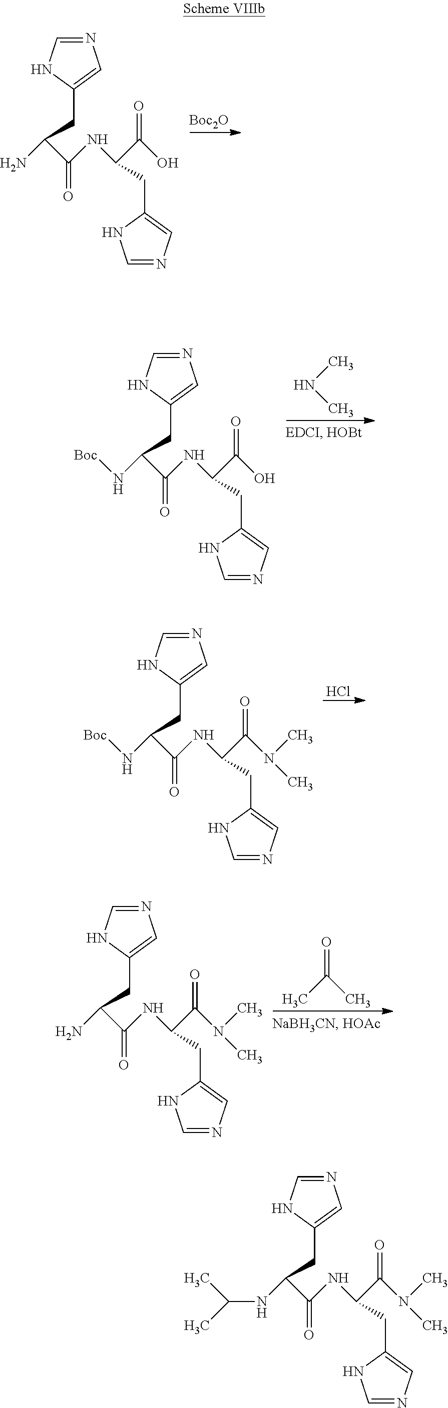

### Consolidatory Formula/Mechanism

This Formula consolidates prior formulas to present some preferred embodiments of this disclosure. These structures share the common motif:

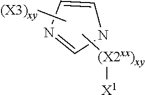

especially:

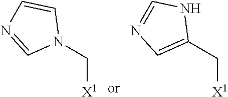

Wherein each X3 is, independently at each point of use, absent, or hydrogen, or alkyl, or substituted alkyl (non-limiting example: CF₃), or deuterated alkyl (non-limiting example: CD₃), or aminoalkyl, or thioalkyl, or alkoxy, or haloalkyl, or haloalkoxy, or hydroxyalkyl, or any atom or isotope permitted by valence (including any accompanying hydrogens by valence e.g. (non-limiting) OH, NH₂, SH, SiH₃, PH₂ etc.), for example deuterium, or halogen, or fluorine;
Each X2^{xx} is, independently at each point of use, selected from a single bond, O, S, Se, NXP, PXP, BXP, C(X^{p})₂ or Si(X^{p})₂, wherein each X^{p} is, independently at each point of use, selected from a constituent group of X3 (defined earlier);
xx and xy are each independently selected from 0, 1, 2, 3, 4, 5;
X1 is

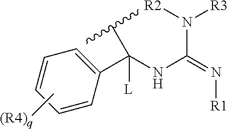

wherein Markush symbols are as defined previously for Formula (I), or

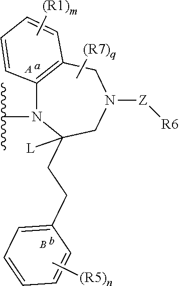

wherein Markush symbols are as defined previously for Formula (II), or

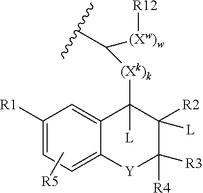

wherein Markush symbols are as defined previously for Formula (III), or

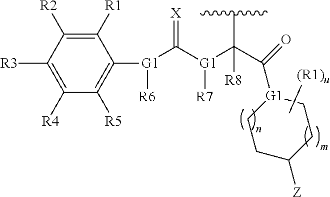

wherein Markush symbols are as defined previously for Formula (IV), or

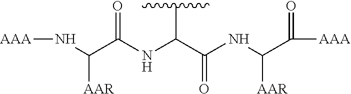

wherein each AAR is, independently at each point of use, an amino acid side chain, optionally (but not restrictively) the side chain of an amino acid coded for by the genetic code, each AAA is, independently at each point of use, an amino acid (proteingenic or non-proteingenic) or a chain of amino acids (proteingenic, or non-proteingenic or a combination thereof) linked by peptide bonds, preferably not exceeding 300 amino acid residues, optionally an amino acid sequence coded for by a component of a genome, optionally an IF1 protein/fragment (or sequence variant thereof), preferably an amino acid sequence falling under Formula (VIII) and/or presented in FIG. 10, optionally wherein one or more of the amino acids have a post-translational modification(s), and/or a modification/manipulation to increase plasma stability, wherein such strategies are well known to those of the art e.g. swopping stereochemistry (D instead of L) of one or more amino acids, optionally with a modification at their N and/or C-terminal ends as disclosed elsewhere herein.

Whilst it is true that compounds of Formula (V) don't have this imidazole structural motif, it is also true that they aren't particularly potent inhibitors of F₁F₀ ATP hydrolysis.

IF1 protein monomers and IF1 protein dimers can inhibit F₁F₀ ATP hydrolysis because their F₁F₀ ATP hydrolysis domain is exposed. Higher IF1 protein oligmers (>dimer) cannot inhibit F₁F₀ ATP hydrolysis because their F₁F₀ ATP hydrolysis domain is buried in the IF1 oligomer, thence higher oligomerization (>dimer) sequesters IF1 protein from inhibiting F₁F₀ ATP hydrolysis, and this is the basis to the pH dependence of IF1 protein inhibition of F₁F₀ ATP hydrolysis: higher pH=IF1 higher (>dimer) oligomerization and low F₁F₀ ATP hydrolysis inhibition, lower pH=higher (>dimer) IF1 oligomers break up and high F₁F₀ ATP hydrolysis inhibition. Without wishing to be restricted by theory, how this imidazole motif enables a compound to inhibit F₁F₀ ATP hydrolysis is that it interacts with, and locates the compound to, one or more histidines in the pH dependence motif of IF1 protein (FIG. 10B) and its location here blocks the higher oligomerization (>dimer) of IF1 protein, without blocking the F₁F₀ ATP hydrolysis domain of IF1 protein, and so there are more free IF1 protein monomers and dimers, thence more inhibition of F₁F₀ ATP hydrolysis. So, the compound interacts with, and exerts its action through, IF1 protein rather than ATP synthase. The compound can reduce F₁F₀ ATP synthesis, but this is by uncoupling rather than by interaction with ATP synthase and direct inhibition of F₁F₀ ATP synthesis (FIG. 17, and its legend, in PCT/EP2018/069175). Componentry to this disclosure is any compound(s) that interacts with IF1 protein, optionally the “pH dependence motif” of IF1 protein (FIG. 10B), optionally wherein the compound(s) has an imidazole motif, or analogue thereof, to reduce/prevent/stop the higher (>dimer) oligomerization of IF1 protein, optionally to treat/prevent/ameliorate/combat one or more of the diseases or disorders or physiological processes or sub-optimalities mentioned herein, optionally cancer and/or aging.

With a compound(s) of this disclosure bound, IF1 protein can still inhibit F₁F₀ ATP hydrolysis, but with the compound bound, IF1 protein cannot higher (>dimer) oligomerize and so there is more free IF1 monomer/dimer and so greater inhibition of F₁F₀ ATP hydrolysis. Thence the size/shape of the compound is paramount, such that is blocks/interferes with the higher (>dimer) oligomerization, and not the inhibitory domain, of IF1 protein. The higher (>dimer) oligomerization domain (or part thereof, or modified form thereof) of IF1 protein, without the inhibitory domain of IF1 protein attached, is componentry to this disclosure (FIG. 10).

A method to find a compound of this disclosure is to assess whether a compound reduces/prevents/stops the higher (>dimer) oligomerization of IF1 protein at alkaline pH (e.g. pH 8). If so, in an optional but preferable 2^{nd} step, it is assessed whether the compound doesn't (necessary) block IF1 protein inhibition of F₁F₀ ATP hydrolysis in a Sub-Mitochondrial Particle (SMP) assay, optionally conducted at pH 6.7. In a 3^{rd} step, or as an alternative 2^{nd} step, it is assessed whether the compound enhances (preferable) IF1 protein inhibition of F₁F₀ ATP hydrolysis in a Sub-Mitochondrial Particle (SMP) assay, optionally conducted at pH 8, optionally wherein action through IF1 protein, rather than direct action upon ATP synthase, can be assayed by observing the effect of removing endogenous IF1 protein from the SMP assay. At alkaline pH (e.g. pH 8), if there is much less F₁F₀ ATP hydrolysis inhibition with much less IF1 protein in the assay, then the compound is acting upon F₁F₀ ATP hydrolysis via interaction with IF1 protein rather than directly upon ATP synthase. If a compound hits criterion/criteria in one or more of these steps, and most preferably in all 3 of these steps, then it is a compound of this disclosure. In some embodiments a screening method is employed using one or more of these steps, wherein a number of compounds are tested in this method to find one or more compounds that reduces/prevents/stops the higher (>dimer) oligomerization of IF1 protein at alkaline pH (e.g. pH 8) and increases IF1 protein inhibition of F₁F₀ ATP hydrolysis at alkaline pH (e.g. pH 8). The 1^{st} step of this method is especially suited to high throughput screening, and only those compounds that pass the 1^{st} step need be entered into the more technically/time demanding 2^{nd} and/or 3^{rd} steps. In some embodiments one or more of these steps is performed at the European Lead Factory.

### Definitions Used to Specify Formulas (I), (II), (III), (IV), (V), (VI), (VII) and (VIII)

The initial definition provided for a group or term herein applies to that group or term throughout the present specification, individually or as part of another group, unless otherwise indicated.

The term “alkyl” refers to straight or branched chain hydrocarbon groups having 1 to 21 carbon atoms, preferably 1 to 8 carbon atoms. Lower alkyl groups, that is, alkyl groups of 1 to 4 carbon atoms, are most preferred.

The term “substituted alkyl” refers to an alkyl group as defined above having one, two, three, or four substituents independently selected from the group consisting of PH2, deuterium, halogen, trifluoromethyl, alkenyl, alkynyl, nitro, cyano, CH, keto (═O), ORₐ, SRₐ, NRₐR_{b}, NRₐSO₂, NRₐSO₂R_{c}, SO₂R_{c}, SO₂NRₐR_{b}, CO₂Rₐ, C(═O)Rₐ, C(═O)NRₐR_{b}, OC(═O)Rₐ, —OC(═O)NRₐR_{b}, NRₐC(═O)R_{b}, NRₐCO₂R_{b}, ═N—OH, ═N—O-alkyl, aryl, heteroaryl, heterocyclo and cycloalkyl, wherein Rₐ and R_{b} are independently selected from hydrogen, alkyl, alkenyl, cycloalkyl, heterocyclo, aryl, and heteroaryl, and R_{c} is selected from hydrogen, alkyl, cycloalkyl, heterocyclo aryl and heteroaryl. When a substituted alkyl includes an aryl, heterocyclo, heteroaryl, or cycloalkyl substituent, said ringed systems are as defined below and thus may in turn have zero to four independently selected substituents (preferably 0-2 substituents), also as defined below. When either Rₐ, R_{b} or R_{c} is an alkyl, said alkyl may optionally be substituted with 1-2 (selected independently) of PH₂, deuterium, halogen, trifluoromethyl, alkenyl, alkynyl, nitro, cyano, CH, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and/or NHCO₂(alkyl).

“Alkyl” when used in conjunction with another group such as in arylalkyl refers to a substituted alkyl in which at least one of the substituents is the specifically named group. For example, the term arylalkyl includes benzyl, or any other straight or branched chain alkyl having at least one aryl group attached at any point of the alkyl chain. As a further example, the term carbamylalkyl includes the group —(CH₂), —NH—C(═O)alkyl, Wherein n is 1 to 12.

The term “alkenyl” refers to straight or branched chain hydrocarbon groups having 2 to 21 carbon atoms and at least one double bond. Alkenyl groups of 2 to 6 carbon atoms and having one double bond are most preferred.

The term “alkynyl” refers to straight or branched chain hydrocarbon groups having 2 to 21 carbon atoms and at least one triple bond. Alkynyl groups of 2 to 6 carbon atoms and having one triple bond are most preferred.

The term “alkylene” refers to bivalent straight or branched chain hydrocarbon groups having 1 to 21 carbon atoms, preferably 1 to 8 carbon atoms, e.g., {—CH₂—}ₙ, Wherein n is 1 to 12, preferably 1-8. Lower alkylene groups, that is, alkylene groups of 1 to 4 carbon atoms, are most preferred. The terms “alkenylene” and “alkynylene” refer to bivalent radicals of alkenyl and alkynyl groups, respectively, as defined above.

When reference is made to a substituted alkylene, alkenylene, or alkynylene group, these groups are substituted with one to four substituents as defined above for alkyl groups. A substituted alkylene, alkenylene, or alkynylene may have a ringed substituent attached in a spiro fashion as in

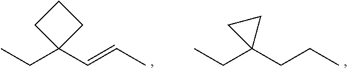

and so forth.

The term “alkoxy” refers to an alkyl or substituted alkyl group as defined above having one, two or three oxygen atoms (—O—) in the alkyl chain. For example, the term “alkoxy” includes the groups-O-C₁₋₁₂alkyl, —C₁₋₆alkylene-O—C₁₋₆alkyl, —C₁₋₄alkylene-O-phenyl, and so forth.

The term “thioalkyl” or “alkylthio” refers to an alkyl or substituted alkyl group as defined above having one or more sulphur (—S—) atoms in the alkyl chain. For example, the term “thioalkyl” or “alkylthio” includes the groups —(CH₂)ₙ-S-CH₂aryl, —(CH₂)ₙ-S-aryl, etc, etc.

The term “aminoalkyl” or “alkylamino” refers to an alkyl or substituted alkyl group as defined above having one or more nitrogen (—NR′—) atoms in the alkyl chain. For example, the term “aminoalkyl” includes the groups —NR′—C₁₋₁₂alkyl and —CH₂—NR′-aryl, etc. (where R′ is hydrogen, alkyl or substituted alkyl as defined above.) “Amino” refers to the group-NH₂.

When a subscript is used as in C₁₋₈alkyl, the subscript refers to the number of carbon atoms the group may contain. Zero when used in a subscript denotes a bond, e.g., C₀₋₄ alkyl refers to a bond or an alkyl of 1 to 4 carbon atoms. When used with alkoxy, thioalkyl or aminoalkyl, a subscript refers to the number of carbon atoms that the group may contain in addition to heteroatoms. Thus, for example, monovalent. C₁₋₂aminoalkyl includes the groups —CH₂—NH₂, —NH—CH₃, —(CH₂)₂—NH₂, —NH—CH₂—CH₃, —CH₂—NH₂—CH₃, and —N—(CH₃)₂. A lower aminoalkyl comprises an aminoalkyl having one to four carbon atoms.

The alkoxy, thioalkyl, or aminoalkyl groups may be monovalent or bivalent. By “monovalent” it is meant that the group has a valency (i.e., power to combine with another group), of one, and by “bivalent” it is meant that the group has a valency of two. For example, a monovalent alkoxy includes groups such as —O-C₁₋₁₂alkyl, —C₁₋₆alkylene-O—C₁₋₆alkyl, etc., whereas a bivalent alkoxy includes groups such as —O—C₁₋₂alkylene-, —C₁₋₆alkylene-O—C₁₋₆alkylene-, etc.

The term “acyl” refers to a carbonyl

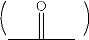

linked to an organic group i.e.

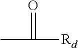

wherein R_{d} may be selected from alkyl, alkenyl, substituted alkyl, substituted alkenyl, aryl, heterocyclo, cycloalkyl, or heteroaryl, as defined herein.

The term “alkoxycarbonyl” refers to a group having a carboxy or ester group

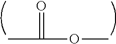

linked to an organic radical, i.e.,

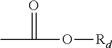

Wherein R_{d} is as defined above for acyl.

The term “carbamyl” refers to a functional group in which a nitrogen atom is directly bonded to a carbonyl, i.e., as in —NRₑC(═O)R_{f} or —C(═O)NRₑR_{f}, wherein Rₑ and R_{f} can be hydrogen, alkyl, substituted alkyl, alkenyl, substituted alkenyl, alkoxy, cycloalkyl, aryl, heterocyclo, or heteroaryl, or they may join to form a ring.

The term “sulfonyl” refers to a sulphoxide group (i.e., —S(O)₁₋₂) linked to an organic radical R_{c}, as defined above.

The term “sulfonamide” or “sulfonamido” refers to the group —S(O)₂NRₑR_{f}, wherein Rₑ and R_{f} are as defined above. Preferably when one of Rₑ and R_{f} is optionally substituted heteroaryl or heterocyclo (as defined below), the other of Rₑ and R_{f} is hydrogen or alkyl.

The term “cycloalkyl” refers to fully saturated and partially unsaturated hydrocarbon rings of 3 to 9, preferably 3 to 7 carbon atoms. The term “cycloalkyl” includes such rings having zero to four substituents (preferably 0-2 substituents), independently selected from the group consisting of OH, SH, PH2, deuterium, halogen, alkyl, substituted alkyl (e.g., trifluoromethyl), alkenyl, substituted alkenyl, alkynyl, nitro, cyano, CH, keto, amino, alkoxy, hydroxy, methoxy, haloalkoxy, OCF₃, OR_{d}, SR_{d} NR_{d}Rₑ NR_{c}SO₂, NR_{c}SO₂Rₑ, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, heterocyclo, a 4 to 7 membered carbocyclic ring, and a five or six membered ketal, e.g., 1,3-dioxolane or 1,3-dioxane, wherein R_{c}, R_{d} and Rₑ are defined as above. The term “cycloalkyl” also includes such rings having a phenyl ring fused thereto or having a carbon-carbon bridge of 3 to 4 carbon atoms. Additionally, when a cycloalkyl is substituted with a further ring, i.e., aryl, arylalkyl, heteroaryl, heteroarylalkyl, heterocyclo, heterocycloalkyl, cycloalkylalkyl, or a further cycloalkyl ring, such ring in turn may be substituted with one to two of C₀₋₄alkyl optionally substituted with one or more groups independently selected from OH, SH, PH2, halogen, trifluoromethyl, alkenyl, alkynyl, nitro, cyano, CH, keto (═O), amino, alkoxy, hydroxy, methoxy, haloalkoxy, OCF₃, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The term “aryl” refers to phenyl, biphenyl, 1-naphthyl, 2-naphthyl, and anthracenyl, with phenyl being preferred. The term “aryl” includes such rings having zero to four substituents (preferably 0-2 substituents), independently selected from the group consisting of deuterium, OH, SH, PH2, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, OR_{d}, SR_{d}, NR_{d}Rₑ, NR_{d}SO₂, NRₐSO₂R_{c}, C(═O)H, acyl, —CO₂H, alkoxycarbonyl, carbamyl, sulfonyl, sulfonamide, —OC(═O)R_{d}, heteroaryl, heterocyclo, cycloalkyl, phenyl, benzyl, napthyl, including phenylethyl, phenyloxy, and phenylthio, wherein R_{c}, R_{d} and Re are defined as above. Additionally, two substituents attached to an aryl, particularly a phenyl group, may join to form a further ring such as a fused or spiro-ring, e.g., cyclopentyl or cyclohexyl or fused heterocyclo or heteroaryl. When an aryl is substituted with a further ring, such ring in turn may be substituted with one to two of C₀₋₄alkyl optionally substituted one or more groups independently selected from deuterium, SH, PH₂, halogen, trifluoromethyl, alkenyl, alkynyl, nitro, cyano, amino, alkoxy, hydroxy, methoxy, haloalkoxy, OCF₃, CH, keto (═O), OH, O(alkyl), phenyloxy, benzyloxy, SH, S(alkyl), NH₂, NH(alkyl), N(alkyl)2, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

The term “heterocyclo” refers to substituted and unsubstituted non-aromatic 3 to 7 membered monocyclic groups, 7 to 11 membered bicyclic groups, and 10 to 15 membered tricyclic groups, in which at least one of the rings has at least one heteroatom selected from O, S and N. Each ring of the heterocyclo group containing a heteroatom can contain one or two oxygen or sulfur atoms and/or from one to four nitrogen atoms provided that the total number of heteroatoms in each ring is four or less, and further provided that the ring contains at least one carbon atom. The fused rings completing bicyclic and tricyclic groups may contain only carbon atoms and may be saturated, partially saturated, or unsaturated. The nitrogen and sulfur atoms may optionally be oxidized and the nitrogen atoms may optionally be quaternized. The heterocyclo group may be attached at any available nitrogen or carbon atom. The heterocyclo ring may contain zero to four substituents (preferably 0-2 substituents), independently selected from the group consisting of deuterium, OH, SH, PH2, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, keto, ORₐ, SRₐ, NR_{d}Rₑ, NRₐSO₂, NRₐSO₂R_{c}, SO₂Rₐ, C(═O)H, acyl, —CO₂H, alkoxycarbonyl, carbamyl, sulfonyl, sulfonamide, —OC(═O)R_{d}, ═N—OH, ═N—O-alkyl, aryl, heteroaryl, cycloalkyl, a five or six membered ketal, e.g., 1,3-dioxolane or 1,3-dioxane, or a monocyclic 4 to 7 membered non aromatic ring having one to four heteroatoms, wherein R_{c}, R_{d} and Rₑ are defined as above. The term “heterocyclo” also includes such rings having a phenyl ring fused thereto or having a carbon-carbon bridge of 3 to 4 carbon atoms. Additionally, when a heterocyclo is substituted with a further ring, i.e., aryl, arylalkyl, heteroaryl, heteroarylalkyl, cycloalkyl, cycloalkylalkyl, heterocycloalkyl, or a further heterocyclo ring, such ring in turn may be substituted with one to two of C₀₋₄alkyl optionally substituted with one or more groups independently selected from deuterium, SH, PH₂, halogen, trifluoromethyl, alkenyl, alkynyl, nitro, cyano, amino, alkoxy, hydroxy, methoxy, haloalkoxy, OCF₃, CH, keto (═O), OH, O(alkyl), phenyloxy, benzyloxy, SH, S(alkyl), NH₂, NH(alkyl), N(alkyl)₂, NHSO₂, NHSO₂(alkyl), SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic groups include azetidinyl, pyrrolidinyl, oxetanyl, imidazolinyl, oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5 to 7 membered monocyclic groups, 9 or 10 membered bicyclic groups, and 11 to 14 membered tricyclic groups which have at least one heteroatom selected from O, S and N in at least one of the rings. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. The heteroaryl ring system may contain zero to four substituents (preferably 0-2 substituents), independently selected from the group consisting of deuterium, OH, SH, PH2, halo, alkyl, substituted alkyl (e.g., trifluoromethyl), alkenyl, substituted alkenyl, alkynyl, nitro, cyano, amino, alkoxy, hydroxy, methoxy, haloalkoxy, OCF₃, CH, OR_{d}, SR_{d}, NR_{d}Rₑ, NR_{d}SO₂, NR_{d}SO₂R_{c}, SO₂R_{d}, C(═O)H, acyl, —CO₂H, alkoxycarbonyl, carbamyl, sulfonyl, sulfonamide, —OC(═O)Rₐ, heterocyclo, cycloalkyl, aryl, or a monocyclic 4 to 7 membered aromatic ring having one to four heteroatoms, including phenylethyl, phenyloxy, and phenylthio, wherein R_{c}, R_{d} and Rₑ are defined as above. Additionally, when a heteroaryl is substituted with a further ring, i.e., aryl, arylalkyl, heterocyclo, heterocycloalkyl, cycloalkyl, cycloalkylalkyl, heteroarylalkyl, or a further heteroaryl ring, such ring in turn may be substituted with one to two of C₀₋₄ alkyl optionally substituted with one or more groups independently selected from deuterium, PH₂, halogen, trifluoromethyl, alkenyl, alkynyl, nitro, cyano, amino, alkoxy, hydroxy, methoxy, haloalkoxy, OCF₃,CH, keto (═O), OH, O(alkyl), phenyloxy, benzyloxy, SH, S(alkyl), NH₂, NH(alkyl), N(alkyl)₂, NHSO₂, NHSO₂(alkyl)ₙ, SO₂(alkyl), SO₂NH₂, SO₂NH(alkyl), CO₂H, CO₂(alkyl), C(═O)H, C(═O)alkyl, C(═O)NH₂, C(═O)NH(alkyl), C(═O)N(alkyl)₂, OC(═O)alkyl, —OC(═O)NH₂, —OC(═O)NH(alkyl), NHC(═O)alkyl, and NHCO₂(alkyl).

Exemplary monocyclic heteroaryl groups include pyrrolyl, pyrazolyl, pyrazolinyl, imidazolyl, oxazolyl, isoxazolyl, thiazolyl

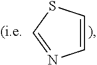

thiadiazolyl, isothiazolyl, furanyl, thienyl, oxadiazolyl, pyridyl, pyrazinyl, pyridinyl, pyrimidinyl, pyridazinyl, triazinyl and the like.

Exemplary bicyclic heteroaryl groups include indolyl, benzothiazolyl, benzodioxolyl, benzoxaxolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridinyl, dihydroisoindolyl, tetrahydroquinolinyl and the like.

Exemplary tricyclic heteroaryl groups include carbazolyl, benzidolyl, phenanthrollinyl, acridinyl, phenanthridinyl, xanthenyl and the like.

When the term “unsaturated” is used herein to refer to a ring or group, the ring or group may be fully unsaturated or partially unsaturated.

The phrase “optionally substituted” is intended to include substituted or unsubstituted possibilities. Accordingly, the phrase “each group of which may be optionally substituted” means that each group includes both substituted and unsubstituted groups. Where a list of substituents is specified for a group, unless specified otherwise, all substituent combinations permitted by valence are contemplated.

The use of the phrase “Where valence allows” means that the groups may be substituted only to the degree and nature allowed by valency of the group. This is commonly understood by those of skill in the art. For example, a hydrogen substituent cannot be further substituted nor can a phenyl group be directly substituted by an oxo group due to limits on valency.

The term “substituted amino” refers to a group of the formula —NZ²Z³ wherein Z² is hydrogen, alkyl, cycloalkyl, aryl, arylalkyl, (cycloalkyl)alkyl, morpholinylalkyl, heterocyclo or (heterocyclo)alkyl and Z³ is hydrogen, alkyl, cycloalkyl, aryl, arylalkyl, haloalkyl, hydroxyalkyl, alkoxyalkyl, thioalkyl, (cycloalkyl)alkyl or hydroxyalkyl further substituted with a carboxylic ester and/or carboxylic acid, with the proviso that when Z² is hydrogen, then Z³ is other than hydrogen; or Z² and Z³ taken together with the nitrogen atom to which they are attached are 1-pyrrolidinyl, 1-piperidinyl, 1-azepinyl, 4-morpholinyl, 4-thiamorpholinyl, 1-piperazinyl, 4-alkyl-1-piperazinyl, 4-arylalkyl-1-piperazinyl, 4-diarylalkyl-1-piperazinyl; or 1-pyrrolidinyl, 1-piperidinyl, 1-azepinyl substituted with one or more groups independently selected from alkyl, alkoxy, alkylthio, halo, trifluoromethyl or hydroxy.

Hydroxy, hydroxyl and —OH are used interchangeably herein.

The term “heterocyclo” or “hetero” also includes such monocyclic and bicyclic rings wherein an available carbon atom is substituted with a (C₁-C₄)-alkyl, aryl, (C₁-C₄)-alkylthio, (C₁-C₄)-alkoxy, halo, nitro, keto, cyano, CH, hydroxy, azo, thiazo, amino, —NH—(C₁-C₄)-alkyl, —N((C₁-C₄)-alkyl)₂, —CF₃, (aminoester)alkyl, carboxylic acid, carboxylic ester, —OCHF₂ or (C₁-C₄)-alkoxy further substituted with a carboxylic acid or such monocyclic and bicyclic rings wherein two or three available carbons have substituents independently selected from methyl, methoxy, methylthio, halo, —CF₃, nitro, hydroxy, amino and —OCHF₂.

Herein, when “carboxyl” or “carboxylic acid” is used, this can mean-C(O) OH but can also refer to carboxylic ester or ester, which encompasses —OC(O)ORₑₛₜₑᵣ and —C(O)ORₑₛₜₑᵣ wherein Rₑₛₜₑᵣ is selected from hydrogen, deuterium, alkyl, alkenyl, alkoxy, thioalkyl, aminoalkyl, cycloalkyl, haloalkyl, haloalkoxy, aryl, heteroaryl or heterocyclo, each of which may be optionally substituted, as defined herein.

Use of “aryl” herein encompasses “aryloxy”, which refers to —O-aryl, wherein aryl is selected from prior definition of aryl specified herein. Use of “heteroaryl” herein encompasses “heteroaryloxy”, which refers to —O-heteroaryl, wherein heteroaryl is selected from prior definition of heteroaryl specified herein. Use of “heterocyclo” herein encompasses “hetereocyclooxy”, which refers to —O-heterocyclo, wherein heterocyclo is selected from prior definition of heterocyclo specified herein.

Use of “ether” herein encompasses —ORₑₜₕₑᵣ wherein Rₑₜₕₑᵣ is selected from hydrogen, deuterium, alkyl, alkenyl, alkoxy, thioalkyl, aminoalkyl, cycloalkyl, haloalkyl, haloalkoxy, aryl, heteroaryl or heterocyclo, each of which may be optionally substituted, as defined herein.

Alkylaryl groups are aryl groups substituted with one or more alkyl groups wherein the alkyl groups optionally carry additional substituents and the aryl groups are optionally substituted. Specific alkylaryl groups include alkyl-substituted phenyl groups such as methylphenyl.

At various places herein, substituents of compounds are disclosed in groups or in ranges. It is specifically intended that this disclosure include each and every individual sub-combination of the members of such groups and ranges. For example, the term “C₁₋₃alkyl” is intended to include C₁alkyl (methyl), C₂alkyl (ethyl), C₃alkyl.

It should be understood that each numerical range within this disclosure also contemplates every possible sub-range within said range. For example, the description of a group having the range of 1-10 carbons also contemplates a group possessing a sub-range of, for (non-limiting) example, 1-3, 1-5, 1-8, or 2-3, 2-5, 2-8, 3-4, 3-5, 3-7, 3-9, 3-10, etc., carbons. Thus, the range 1-10 should be understood to represent the outer boundaries of the range, within which many possible sub-ranges are also contemplated. Additional examples, contemplating ranges in other contexts, can be found in this disclosure wherein such ranges include analogous sub-ranges within.

### Stereoisomers

All stereoisomers of Formula [X], such as those, for example, which may exist due to asymmetric carbons, including enantiomeric forms (which may exist even in the absence of asymmetric carbons) and diastereomeric forms, are contemplated and within the scope of this disclosure. Individual stereoisomers of the compounds of this disclosure may, for example, be substantially free of other isomers, or may be admixed, for example, as racemates or with all other or other selected, stereoisomers. The chiral centers of the present disclosure can have the S or R configuration as defined by the IUPAC 1974 Recommendations.

This disclosure encompasses the use of stereomerically pure forms of a compound(s) of this disclosure, as well as the use of mixtures of those forms. For example, mixtures comprising equal or unequal amounts of the enantiomers of a compound(s) of Formula [X], for example a compound(s) of Formula (I), may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725(1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

For a compound of this disclosure: the disclosure contemplates all combinations, pure forms, forms of high purity, particle sizes, polymorphs, crystals, co-crystals, self-aggregates, colloids, colloid forms as described in [257-258] and/or other paper(s) from the same research group, light scattering forms, metabolites, derivatives, isotopologues, geometric/conformational isomers, rotamers, atropisomers, atropenantiomers, stereoisomers, optically active forms, racemates, scalemates, epimers, tautomers, keto-enol tautomers, cis- and trans-isomers, E and Z isomers, R- and S-enantiomers, diastereomers, isomers, (D)-isomers, (L)-isomers, the racemic mixtures thereof, other mixtures thereof, isomers, isomer substantially free of other isomer(s), mixtures of isomers and isotopic variants (e.g. deuterium in place of hydrogen in some or all places upon the molecule{s}) as falling within the scope of this disclosure. All such isomers, as well as mixtures thereof, are intended to be included in this disclosure. As well as analogues and pharmaceutically/physiologically acceptable salts/ethers/esters/solvates/hydrates/solvates of salts/chelates/complexes/metal complexes/mixtures/prodrugs/particles/radionuclides/derivatives/carriers/crystalline forms/isomorphic crystalline forms/crystals/co-crystals/clathrates/liposomes/fusogenic liposomes/mixed_liposomes/exosomes/vesicles/chylomicrons/lipospheres/nanoparticles/microparticles/microbubbles/micronized forms/polymer matrices/drug-polymer conjugates/microspheres/micelles/mixed micelles of surfactants/mixed micelles of surfactant-phospholipid/niosomes/oleosomes/ethosomes/Lipid Based Nanoparticles (LNPs)/Nanoparticle-Stabilized Nanocapsules (NPSCs)/solid lipid nanoparticles/nanostructured lipid carriers/millicapsules/microcapsules/nanocapsules/millispheres/microspheres/nanospheres/miniparticles/milliparticles/microparticles/nanoparticles/microemulsions/nanoemulsions/sponges/microsponges/cyclod extrins/compositions/formulations/doses/combinations/N-oxides/acid salt hydrates thereof (and combination[s] thereof). Contemplated is a compound of this disclosure bound to albumin. Unless indicated otherwise, chemical structures and graphical representations of compounds herein encompass all stereoisomers, racemates, scalemates, relative proportions/combinations of R and S stereoisomers and optically active or inactive forms. Optically active forms may be prepared by resolution of racemic forms or by synthesis from optically active starting materials. Many geometric isomers of C═C double bonds, C═N double bonds, ring systems, and the like can also be present in the compounds, and all such stable isomers are contemplated in the present disclosure. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. The present disclosure includes all cis, trans, syn, anti, entgegen (E), and zusammen (Z) isomers as well as the appropriate mixtures thereof. Compounds of the present disclosure, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, in so far as they may exist, are included within this disclosure. Additionally, the compounds disclosed herein can exist in unsolvated as well as solvated forms with pharmaceutically acceptable solvents such as water, ethanol, and the like: all componentry to the present disclosure. The present disclosure is not limited to any particular mechanism, nor to any understanding of the action of the agent(s) being administered.

When a chiral center(s) exists in a compound of this disclosure, it is to be understood that this disclosure encompasses all possible stereoisomers. Each stereogenic carbon or sulfur may be of R or S configuration. Although the specific compounds exemplified in this disclosure may be depicted in a particular configuration, compounds having the opposite stereochemistry at any given chiral center, or mixtures thereof, are also envisioned.

When epimerization is relevant in vivo, administering an enantiomeric excess of one stereoisomer to a subject in order to administer the opposite stereoisomer to this same subject is contemplated by, and componentry to, this disclosure. Administering an enantiomeric excess of a stereoisomer to administer an effective amount of the opposite stereoisomer is contemplated by, and componentry to, this disclosure. For example, one of the art will recognize that administration of a compound in its (R) form is partially/fully equivalent, for compounds that undergo R to S epimerization in vivo, to administration of the compound in its(S) form.

The present disclosure includes all isotopes of all atoms occurring in the present compounds. So, this disclosure embraces isotopically labeled compounds of the disclosure which are identical to those recited herein, except that one or more atoms are replaced by an atom having an atomic mass and/or mass number different from the atomic mass and/or mass number usually found in nature. Isotopes include those atoms having the same atomic number but different mass numbers. By way of general example and without limitation, examples of isotopes that can be incorporated into compounds of this disclosure include isotopes of hydrogen, carbon, nitrogen, oxygen, phosphorus, sulfur, fluorine and chlorine, such as ²H, ³H, ¹³C, ¹⁴C, ¹⁵N, ¹⁷O, ¹⁸O, ³¹P, ³²P, ³³S, ³⁴S, ³⁵S, ³⁶S, ¹⁸F, and ³⁶Cl, respectively. Isotopically labeled compounds of the present disclosure can generally be prepared by following procedures analogous to those disclosed herein by substituting an isotopically labeled reagent for a non-isotopically labeled reagent. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Substitution with positron emitting isotopes, such as ¹¹C, ¹⁸F, ¹⁵O and ¹³N, can be useful in PET studies for examining substrate receptor occupancy. These radiolabeled compounds can be useful to further determine or measure the effectiveness of the compounds, by characterizing, for example, the site/mode of action, or binding affinity to pharmacologically important site of action. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Also provided for isotopically labeled compounds described herein are any pharmaceutically acceptable salts, solvates, hydrates or prodrugs, as the case may be.

In cases wherein there are nitrogen atoms (e.g., amines) on compounds of the present disclosure, these may be converted to N-oxides by treatment with an oxidizing agent (e.g., mCPBA and/or hydrogen peroxides) to afford other compounds of this disclosure. Thus, shown/claimed nitrogen atoms are considered to cover both the shown nitrogen and its N-oxide (N→O) derivative.

Membrane permeant derivatives of the compounds of this disclosure are contemplated by and componentry to this disclosure. “Membrane permeant derivative” refers to a chemical derivative of a compound that increases the membrane permeability of the compound. These derivatives are made better able to cross cell membranes because hydrophilic groups are masked to provide more hydrophobic derivatives. Also, the masking groups can be designed to be cleaved from the compound within a cell to make the compound more hydrophilic once within the cell. Because the compound is more hydrophilic than its membrane permeant derivative, it preferentially localizes within the cell (U.S. Pat. No. 5,741,657).

Any metabolite of a compound of this disclosure is a compound of this disclosure. Componentry to this disclosure is any compound of this disclosure, optionally a compound of Formula (I), (II), (III), (IV), (V), (VI), (VII), (VIII), [X], herein, with one or more of its H atoms/isotopes replaced with OH (hydroxyl), for example catalysed/enabled by an enzyme e.g. one or more of a Cytochorome P450 enzyme(s), monoxygenase enzyme(s), hydroxylase enzyme(s) and/or by other relevant enzyme(s) known to those of the art. Optionally, but not restrictively, such a compound(s) of this disclosure can be produced by incubating a compound(s) of this disclosure with microsomes, optionally liver microsomes, wherein this technique is well known to those of the art.

Reference to a compound(s) of Formula (I), (II), (III), (IV), (V), (VI), (VII), (VIII), [X], herein is understood to include reference to every tautomer, enantiomer, mixture of enantiomers, salt, solvate, hydrate, prodrug, chemically protected form, ester, N-oxide, metabolite, crystal, polymorph, co-crystal, clathrate thereof, unless otherwise indicated.

### Salts & Solvates

Throughout the specification, groups and substituents thereof may be chosen by one skilled in the field to provide stable moieties and compounds.

The compounds of Formula [X] form salts which are also within the scope of this disclosure. Reference to a compound of the Formula [X] herein is understood to include reference to salts thereof, unless otherwise indicated. For illustrating, not restricting, example: quaternary ammonium salts of compounds of Formula [X] are componentry to the present disclosure. Generally speaking, salts tend to be more soluble in aqueous or other protonic solvents than are the corresponding free base forms.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, immunogenicity, and/or other problem or complication, commensurate with a reasonable benefit/risk ratio.

As used herein, the term “pharmaceutically acceptable salt” refers to any pharmaceutically acceptable salt (e.g., acid or base) of a compound of the present disclosure which, upon administration to a subject, is capable of providing a compound of this disclosure or an active metabolite or residue thereof. As is known to those of ordinary skill in the art, “salts” of the compounds of the present disclosure may be derived from inorganic or organic acids and bases. For therapeutic use, salts of the compounds of the present disclosure are contemplated as being pharmaceutically acceptable (i.e., non-toxic, physiologically acceptable). However, salts of acids and bases that are non-pharmaceutically acceptable may also find use, for example, in the preparation, isolation or purification of a pharmaceutically acceptable compound. Lists of suitable salts are found in Remington's Pharmaceutical Sciences, 18^{th} Edition, Mack Publishing Company, Easton, Pa. (1990) and Handbook of Pharmaceutical Salts: Properties, Selection, and Use by Stahl and Wermuth (Wiley-VCH, 2002) and refer Berge et al. (1977) “Pharmaceutical Salts”, J. Pharm. Sci. 66:1-19, all hereby incorporated by reference.

The term “salt(s)”, as employed herein, denotes acidic and/or basic salts formed with inorganic and/or organic acids and bases. In addition, when a compound of Formula [x] contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein.

Salts can be prepared in situ during the final isolation and purification of the compounds of this disclosure, or by separately reacting a purified compound of this disclosure in its free base form with a suitable organic or inorganic acid, and isolating the salt thus formed. Salts of the compounds of the Formula [X] may be formed, for example, by reacting a compound of the Formula [X] with an amount of acid or base, such as an equivalent amount, in a medium such as one in which the salt precipitates or in an aqueous medium followed by lyophilization.

The compounds of Formula [X] which contain a basic moiety, such as, but not limited to an amine or a pyridine or imidazole ring, may form salts with a variety of organic and inorganic acids. Exemplary acid addition salts include acetates (such as those formed with acetic acid or trihalo acetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methane-sulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The compounds of Formula [X] which contain an acidic moiety, such as, but not limited to a carboxylic acid, may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis (dehydro-abietyl)ethylenediamine], N-methyl D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e. g., decyl, lauryl, myristyl and stearyl chlorides, bromides and iodides), aralkyl halides (e.g., benzyl and phenethyl bromides), and others.

Exemplary salts include FDA/EMA approved salts for pharmaceutical use.

Compounds of Formula [X], and salts thereof, may exist in their tautomeric form (for example, as an amide or imino ether). All such tautomeric forms are contemplated herein as part of the present disclosure.

It should be understood that solvates (e.g., hydrates) of a compound(s) of Formula [X] are also within the scope of the present disclosure. Herein, the term “solvate” means a compound provided herein or a salt thereof that further includes a stoichiometric or non-stoichiometric amount of solvent (organic or inorganic) bound by non-covalent intermolecular forces (e.g. hydrogen bonding). Where the solvent is water, the solvate is a hydrate. In certain instances the solvate will be capable of isolation, for example when one or more solvent molecules are incorporated in the crystal lattice of the crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution-phase and isolable solvates. Exemplary solvates include, but are not limited to, hydrates, ethanolates, methanolates, and isopropanolates. Methods of solvation are known in the art.

Chelates, metal complexes (metal complexes include calcium, zinc, iron and the like), mixtures, radionuclides and liposomes of Formula [X] are within the scope of this disclosure. In some embodiments, a compound(s) of Formula [X] is anhydrous.

### Fatty Acids

In an embodiment, a therapeutic compound(s) componentry to the present disclosure, for example a compound(s) of Formula [X], is formulated with a pharmaceutically-acceptable fatty acid(s). In an embodiment, the stoichiometry of a compound of Formula [X] to a fatty acid is 1:1, in another embodiment it is 1:2, and in other embodiments it is other stoichiometries/ratios. What constitutes a fatty acid is well known to those of the art, and isn't necessarily limited to the brief specification given herein. A fatty acid is a carboxylic acid with an aliphatic chain, which is either saturated (no C═C double bonds; in some cases with a formula CH₃(CH₂)ₙCOOH where n is a positive integer) or unsaturated (with one or more C═C double bonds, each of which can be in a cis or trans configuration; monounsaturated fatty acids have a single C═C double bond, polyunsaturated fatty acids (PUFA) have more than one C═C double bond, methylene-interrupted polyenes have two or more cis double bonds separated from each other by a single methylene bridge (—CH₂—) (i.e. C═C—C—C═C), conjugated fatty acids have at least one pair of double bonds separated by only a single bond (i.e. C═C—C═C)), which is either branched or (more typically) unbranched, which is optionally substituted. This arrangement confers a fatty acid with a polar, hydrophilic end, and a nonpolar, hydrophobic end that is insoluble in water. Most naturally occurring fatty acids have an unbranched hydrocarbon chain of an even number of carbon atoms, typically between 4 and 28 carbons, and may be attached to functional groups containing oxygen, halogens, nitrogen, and sulfur. Synthetic or non-natural fatty acids may have a hydrocarbon chain of any number of carbon atoms from between 3 and 40 carbons. Where a double bond exists, there is the possibility of either cis or trans geometric isomerism (=E/Z isomerism in IUPAC nomenclature), which significantly affects the molecule's molecular configuration. Cis-double bonds cause the fatty acid chain to bend, an effect that is more pronounced the more cis double bonds there are in a chain. Most naturally occurring fatty acids are of the cis configuration, although the trans form does exist in some natural and partially hydrogenated fats and oils, wherein trans fats have been associated with increased risk of coronary heart disease. Omega (Φ) is the name for the methyl end of a fatty acid and, starting from the omega end, omega-3 (ω-3 or n-3) fatty acids have their first (in some cases only) double bond between the 3^{rd} and 4^{th} carbon atoms from the methyl end, omega-6 (ω-6 or n-6) fatty acids have their first (in some cases only) double bond between the 6^{th} and 7^{th} carbon atoms from the methyl end, omega-7 (ω-7 or n-7) fatty acids have their first (in some cases only) double bond between the 7^{th} and 8^{th} carbon atoms from the methyl end, omega-9 (ω-9 or n-9) fatty acids have their first (in some cases only) double bond between the 9^{th} and 10^{th} carbon atoms from the methyl end. In naming fatty acids a form used is, to illustrate with an example, (18:3, n-3), wherein this fatty acid has 18 carbons in its aliphatic chain, 3 double bonds, the first of which emanates from the 3^{rd} carbon from the methyl (omega, w) end of the chain (i.e. this is an omega-3 fatty acid). To give a further example, (20:5, n-3), wherein this fatty acid has 20 carbons in its aliphatic chain, 5 double bonds, the first of which emanates from the 3^{rd} carbon from the methyl (omega, w) end of the chain (i.e. this is an omega-3 fatty acid). To give yet a further example, (20:3, n-9), wherein this fatty acid has 20 carbons in its aliphatic chain, 3 double bonds, the first of which emanates from the 9^{th} carbon from the methyl (omega, @) end of the chain (i.e. this is an omega-9 fatty acid). Note that these examples haven't been fully specified using this notation because the cis/trans (E/Z) isomerism of their double bond(s) hasn't been specified. Also, note that this naming system is not what is specified by IUPAC, where for one thing, carbons are counted from the carboxyl rather than the methyl end of the aliphatic chain. α-Linolenic acid (ALA) is (18:3, n-3) and in IUPAC nomenclature: (9Z,12Z,15Z)-9,12,15-Octadecatrienoic acid, wherein this fatty acid has 3 double bonds, all Z configuration, at carbons 9, 12, 15 from the carboxyl end, wherein (to revert back away from IUPAC) its first double bond emanates from the 3^{rd} carbon atom from the methyl end.

Examples of fatty acids include, without limitation, Capryllic acid, Caprylic acid, pelargonic acid, Capric acid. Undecylic acid, Lauric acid, Tridecylic acid, Myristic acid, Myristoleic acid, Pentadecyclic acid, Palmitic acid, Palmitoleic acid, Sapienic acid, Margaric acid, Stearic acid, Oleic acid, Elaidic acid, Vaccenic acid, Linoleic acid, Linoelaidic acid, α-Linolenic acid, γ-Linolenic acid, Stearidonic acid, Nonadecylic acid, Arachidic acid, Eicosenoic acid, 11-Eicosenoic acid, Dihomo-γ-linolenic acid, Mead acid, Arachidonic acid, Eicosapentaenoic acid, Heneicosylic acid, Behenic acid, Erucic acid, Docosahexaenoic acid, Tricosylic acid, Lignoceric acid, Nervonic acid, Pentacosylic acid, Cerotic acid, Heptacosylic acid, Montanic acid, Nonacosylic acid, Melissic acid, Henatriacontylic acid, Lacceroic acid, Psyllic acid, Geddic acid, Ceroplastic acid, and Hexatriacontylic acid, α-Linolenic acid, γ-Linolenic acid, omega 3 fatty acid (ALA), omega 3 fatty acid (DHA), omega 3 fatty acid (EPA), omega 6 fatty acid (AA), omega 7 fatty acid (CLA), omega 9 fatty acid (OA), omega 9 hydroxylated fatty acid (R_{A}), Arachidonic acid, [9Z, 11E conjugated Linoleic acid], conjugated linoleic acid, conjugated (9Z,11E)-Linoleic acid (CAS Number 2540-56-9), Docosahexaenoic acid, Eicosapentaenoic acid, Oleic acid, Ricinoleic acid, butyric acid, hexanoic acid, decanoic acid, valeric acid, pentadecan acid, heptanoic acid, docosatetraenoic acid, heptadecanoic acid, paullinic acid, alpha-parinaric acid, calendic acid, docosapentaenoic acid, linolelaidic acid, gadoleic acid, alpha-Eleostearic acid, petroselinic acid, punicic acid, pinolenic acid, rumenic acid, eicosatetraenoic acid, tuberculostearic acid, vernolic acid, prostanoic acid, catalpic acid, jacaric acid, malvalic acid, aleuritic acid, lesquerolic acid, bosseopentaenoic acid, hydnocarpic acid.

In aspects of this embodiment, a saturated or unsaturated fatty acid comprises, e.g., at least 8, at least 10, at least 12, at least 14, at least 16, at least 18, at least 20, at least 22, at least 24, at least 26, at least 28, or at least 30 carbon atoms. In other aspects of this embodiment, a saturated or unsaturated fatty acid comprises, e.g., between 4 and 24 carbon atoms, between 6 and 24 carbon atoms, between 8 and 24 carbon atoms, between 10 and 24 carbon atoms, between 12 and 24 carbon atoms, between 14 and 24 carbon atoms, or between 16 and 24 carbon atoms, between 4 and 22 carbon atoms, between 6 and 22 carbon atoms, between 8 and 22 carbon atoms, between 10 and 22 carbon atoms, between 12 and 22 carbon atoms, between 14 and 22 carbon atoms, or between 16 and 22 carbon atoms, between 4 and 20 carbon atoms, between 6 and 20 carbon atoms, between 8 and 20 carbon atoms, between 10 and 20 carbon atoms, between 12 and 20 carbon atoms, between 14 and 20 carbon atoms, or between 16 and 20 carbon atoms. If unsaturated, the fatty acid may have, e.g., 1 or more, 2 or more, 3 or more, 4 or more, 5 or more, or 6 or more double bonds.

In another embodiment, an adjuvant may comprise one kind of pharmaceutically-acceptable fatty acid. In another embodiment, an adjuvant may comprise a plurality of different pharmaceutically-acceptable fatty acids. In aspects of this embodiment, an adjuvant may comprise, e.g., two or more different fatty acids, three or more different fatty acids, four or more different fatty acids, five or more different fatty acids, or six or more different fatty acids.

A pharmaceutically-acceptable fatty acid useful in the pharmaceutical compositions disclosed herein may be a pharmaceutically-acceptable omega fatty acid. Non-limiting examples of an omega fatty acid include an omega-3 fatty acid, an omega-6 fatty acid, an omega-7 fatty acid, an omega-9 fatty acid.

Omega-3 fatty acids (also known as n-3 fatty acids or ω-3 fatty acids) are a family of unsaturated fatty acids that have in common a final carbon-carbon double bond in the n-3 position, that is, the third bond, counting from the methyl end of the fatty acid. Omega-3 fatty acids are “essential” fatty acids because they are vital for normal metabolism and cannot be synthesized by the human body. An omega-3 fatty acid includes, without limitation, Hexadecatrienoic acid (16:3), α-Linolenic acid (18:3), Stearidonic acid (18:4), Eicosatrienoic acid (20:3), Eicosatetraenoic acid (20:4), Eicosapentaenoic acid (20:5), Heneicosapentaenoic acid (21:5), Docosapentaenoic acid (Clupanodonic acid) (22:5), Docosahexaenoic acid (22:6), Tetracosapentaenoic acid (24:5), Tetracosahexaenoic acid (Nisinic acid) (24:6).

Omega-6 fatty acids (also known as n-6 fatty acids or ω-6 fatty acids) are a family of unsaturated fatty acids that have in common a final carbon-carbon double bond in the n-6 position, that is, the sixth bond, counting from the methyl end of the fatty acid. An omega-6 fatty acid includes, without limitation, Linoleic acid (18:2), γ-linolenic acid (18:3), Calendic acid (18:3), Eicosadienoic acid (20:2), Dihomo-γ-linolenic acid (20:3), Arachidonic acid (20:4), Docosadienoic acid (22:2), Adrenic acid (22:4). Docosapentaenoic acid (22:5), Tetracosatetraenoic acid (24:4), and Tetracosapentaenoic acid (24:5).

Omega-7 fatty acids (also known as n-7 fatty acids or ω-7 fatty acids) are a family of unsaturated fatty acids that have in common a final carbon-carbon double bond in the n-7 position, that is, the seventh bond, counting from the methyl end of the fatty acid. An omega-7 fatty acid includes, without limitation, 5-Dodecenoic acid (12:1), 7-Tetradecenoic acid (14:1), 9-Hexadecenoic acid (Palmitoleic acid) (16:1), 11-Decenoic acid (Vaccenic acid) (18:1), 9Z, 11E conjugated Linoleic acid (Rumenic acid) (18:2), 13-Eicosenoic acid (Paullinic acid) (20:1), 15-Docosenoic acid (22:1), and 17-Tetracosenoic acid (24:1).

Omega-9 fatty acids (also known as n-9 fatty acids or ω-9 fatty acids) are a family of unsaturated fatty acids that have in common a final carbon-carbon double bond in the n-9 position, that is, the ninth bond, counting from the methyl end of the fatty acid. An omega-9 fatty acid includes, without limitation, Oleic acid (18:1), Elaidic acid (18:1), Eicosenoic acid (20:1), Mead acid (20:3), Erucic acid (22:1), Nervonic acid (24:1), Ricinoleic acid, ximenic acid (26:1).

A pharmaceutically-acceptable fatty acid useful in the pharmaceutical compositions disclosed herein may be a pharmaceutically-acceptable conjugated fatty acid. Conjugated fatty acids are positional and geometric isomers of polyunsaturated fatty acids in which at least one pair of double bonds are separated by only one single bond. In one aspect of this embodiment, a pharmaceutically-acceptable conjugated fatty acid is, e.g., a C16 conjugated fatty acid, a C18 conjugated fatty acid, a C20 conjugated fatty acid, a C22 conjugated fatty acid, a C24 conjugated fatty acid, a C26 conjugated fatty acid, a C28 conjugated fatty acid or a C30 conjugated fatty acid. In one aspect of this embodiment, pharmaceutically-acceptable conjugated fatty acid is, e.g., a C16-C18 conjugated fatty acid, a C16-C20 conjugated fatty acid, a C16-C22 conjugated fatty acid, a C16-C24 conjugated fatty acid, a C16-C26 conjugated fatty acid, a C16-C28 conjugated fatty acid, a C16-C30 conjugated fatty acid, a C18-C20 conjugated fatty acid, a C18-C22 conjugated fatty acid, a C18-C24 conjugated fatty acid, a C18-C26 conjugated fatty acid, a C18-C28 conjugated fatty acid, a C18-C30 conjugated fatty acid, a C20-C22 conjugated fatty acid, a C20-C24 conjugated fatty acid, a C20-C26 conjugated fatty acid, a C20-C28 conjugated fatty acid, a C20-C30 conjugated fatty acid, a C22-C24 conjugated fatty acid, a C22-C26 conjugated fatty acid, a C22-C28 conjugated fatty acid, a C22-C30 conjugated fatty acid, a C24-C26 conjugated fatty acid, a C24-C28 conjugated fatty acid, a C24-C30 conjugated fatty acid, a C26-C28 conjugated fatty acid, a C26-C30 conjugated fatty acid, or C28-C30 conjugated fatty acid.

In another aspect of this embodiment, a pharmaceutically acceptable conjugated fatty acid includes, e.g., a conjugated Linoleic acid, a conjugated Linoelaidic acid, a conjugated α-Linolenic acid, a conjugated γ-Linolenic acid, a conjugated Calendic acid, a conjugated Eicosadienoic acid, a conjugated Stearidonic acid, a conjugated Nonadecylic acid, a conjugated Arachidic acid, a conjugated Dihomo-γ-linolenic acid, a conjugated DocoSadienoic, a conjugated Mead acid, a conjugated Arachidonic acid, a conjugated Eicosapentaenoic acid, a conjugated Adrenic acid, a conjugated Docosapentaenoic acid, a conjugated Heneicosylic acid, a conjugated Tetracosatetraenoic acid, a conjugated Tetracosapentaenoic acid, a conjugated Behenic acid, a conjugated Docosahexaenoic acid, a conjugated Tricosylic acid, a conjugated Lignoceric acid, a conjugated Pentacosylic acid, a conjugated Cerotic acid, a conjugated Heptacosylic acid, a conjugated Montanic acid, a conjugated Nonacosylic acid, a conjugated Melissic acid, a conjugated Henatriacontylic acid, a conjugated Lacceroic acid, a conjugated Psyllic acid, a conjugated Geddic acid, a conjugated Ceroplastic acid, a conjugated Hexatriacontylic acid, or any combination thereof.

A pharmaceutically-acceptable fatty acid useful in the pharmaceutical compositions disclosed herein may be a pharmaceutically-acceptable conjugated linoleic acid (CLA). Conjugated linoleic acid (CLA) refers to a group of at least 28 positional and geometric isomers of the omega-6 essential fatty acid linoleic acid (cis-9, cis-12, octadecadienoic acid). The double bonds of CLAS are conjugated, with only one single bond between them. Virtually all cis- and trans-isomeric combinations of CLA have been identified. A CLA includes, without limitation, cis-9, trans-11, octadecadienoic acid (c-9, t-11 CLA), cis-9, cis-11, octadecadienoic acid (c-9, c-11 CLA), trans-9, trans-11, octadecadienoic acid (t-9, t-11 CLA), and trans-9, cis-11, octadecadienoic acid (t-9, c-11 CLA), cis-9, trans-11, conjugated linoleic acid (c-9, t-11 CLA), cis-9, cis 11, conjugated linoleic acid (c-9, c-11 CLA), trans-9, trans 11, conjugated linoleic acid (t-9, t-11 CLA), and trans-9, cis-11, conjugated linoleic acid (t-9, c-11 CLA), cis-10, trans 12, conjugated linoleic acid (c-10, t-12 CLA), cis-10, cis-12, conjugated linoleic acid (c-10, c-12 CLA), trans-10, trans-12, conjugated linoleic acid (t-10, t-12 CLA), and trans-10, cis 12, conjugated linoleic acid (t-10, c-12 CLA), and any combination thereof.

In an aspect of this embodiment, a pharmaceutical composition comprises a compound(s) of Formula [X] and an Omega-3 fatty acid(s). In another aspect of this embodiment, a pharmaceutical composition comprises a compound(s) of Formula [X] and an Omega-6 fatty acid(s). In yet another aspect of this embodiment, a pharmaceutical composition comprises a compound(s) of Formula [X] and an Omega-7 fatty acid(s). In still another aspect of this embodiment, a pharmaceutical composition comprises a compound(s) of Formula [X] and an Omega-9 fatty acid(s). In other aspects, a pharmaceutical composition comprises a compound(s) of Formula [X] and an Omega-3 fatty acid, an Omega-6 fatty acid, an Omega-7 fatty acid, an Omega-9 fatty acid, or any combination thereof. In yet other aspects, a pharmaceutical composition comprises a compound(s) of Formula [X] and C-Linolenic acid, Arachidonic acid, Docosahexaenoic acid, Rumenic acid, or any combination thereof.

In some embodiments, contemplated is a pharmaceutical composition that comprises (or consists of) at least one compound of Formula [X] conjoined (e.g. covalently bound) to at least one fatty acid (optionally a fatty acid(s) listed herein).

In an embodiment, a pharmaceutical composition comprises one or more compounds of Formula [X] and one or more of a fatty acid(s), wherein the fatty acid is an omega-3 fatty acid, an omega-6 fatty acid, an omega-7 fatty acid, an omega-9 fatty acid, or any combination thereof, wherein (if present) the omega-3 fatty acid is Hexadecatrienoic acid (16:3), C-Linolenic acid (18:3), Stearidonic acid (18:4), Eicosa trienoic acid (20:3), Eicosatetraenoic acid (20:4), Eicosa pentaenoic acid (20:5), Heneicosapentaenoic acid (21:5), Docosapentaenoic acid (Clupanodonic acid) (22:5), Docosahexaenoic acid (22:6), Tetracosapentaenoic acid (24:5), Tetracosahexaenoic acid (Nisinic acid) (24:6), or any combination thereof, wherein (if present) the omega-6 fatty acid is Linoleic acid (18:2), Y-linolenic acid (18:3), Calendic acid (18:3), Eicosadienoic acid (20:2), Dihomo-y-linolenic acid (20:3), Arachidonic acid (20:4), Docosadienoic acid (22:2), Adrenic acid (22:4).Docosapentaenoic acid (22:5), Tetracosatetraenoic acid (24:4), and Tetracosapentaenoic acid (24:5), or any combination thereof, wherein (if present) the omega-7 fatty acid is 5-Dodecenoic acid, 7-Tetradecenoic acid, 9-Hexadecenoic acid (Palmitoleic acid), 11-Decenoic acid (Vaccenic acid), 13-Eicosenoic acid (Paullinic acid), 15-Docosenoic acid, 17-Tetracosenoic acid, and 9Z,11E conjugated Linoleic acid (Rumenic acid), or any combination thereof, wherein (if present) the omega-9 fatty acid Oleic acid, Elaidic acid, Eicosenoic acid, Meadacid, Erucic acid, Nervonic acid, and Ricinoleic acid, or any combination thereof.

In an embodiment, a pharmaceutical composition comprises one or more compounds of Formula [X] and one or more of a fatty acid, plus optionally a chemotherapeutic and/or anti-proliferative agent(s) and/or one or more compounds approved for human use, optionally for anti-cancer use, by the United States Food and Drug Administration (FDA) and/or European Medicines Agency (EMA). Optionally, further/alternative constituent(s) are componentry to this composition, wherein the range of options is clear to someone of the art, some of which are disclosed elsewhere herein, and include (without limitation) pharmaceutically-acceptable carriers include vehicles, stabilizers, diluents, additives, auxiliarys or excipients, including buffers, preservatives, tonicity adjusters, salts, demulcents, antioxidants, osmolality adjusting agents, physiological substances, pharmacological substances, bulking agents, emulsifying agents, wetting agents, flavoring agents, or coloring agents, a therapeutically effective amount of a chemotherapeutic or anti-proliferative agent(s), wherein the chemotherapeutic or anti-proliferative agent(s) is selected from the group consisting of an alkylating agent, a platinum agent, an antimetabolite, a topoisomerase inhibitor, an antitumor antibiotic, an aromatase inhibitor, a thymidylate synthase inhibitor, a DNA antagonist, farnesyltransferase inhibitor, a pump inhibitor, a metalloproteinase inhibitor, a ribonucleoside reductase inhibitor, a TNFC, agonist, an endothelin A receptor antagonist, a retinoic acid receptoragonist, an immuno-modulator, a hormonal and antihormonal agent, a photodynamic agent, a tyrosine kinase inhibitor, and any combination thereof.

An aspect of this disclosure is to use a composition(s) componentry to this disclosure, for (non-limiting) example a composition containing a compound(s) of Formula [X] and a fatty acid(s), in a method of treatment of the human or animal body by therapy, for (non-limiting) example to treat/ameliorate/prevent/combat one or more of the diseases/disorders/pathologies/damages/processes mentioned herein, including (non-limiting) aging, signs of aging, diseases of aging, cancer and cachexia. A yet further aspect is to use a composition componentry to this disclosure, for (non-limiting) example a composition containing a compound(s) of Formula [X] and a fatty acid(s), for the manufacture of a medicament for the treatment/amelioration/prevention/combat of body damage, aging and/or a disease/disorder/pathology and/or to enhance/improve body/brain function, optionally for one or more of the therapeutic/beneficial/desirable applications mentioned herein, optionally cancer.

Fatty acids can have anti-cancer activity (e.g. refer [185-186]). In an embodiment, when formulating a composition of a compound(s) of Formula [X] and a fatty acid(s), if this composition is to be used for an anti-cancer use, the fatty acid(s) selected should, in a preferred embodiment, be a fatty acid(s) with the greatest anti-cancer activity, especially the fatty acid(s) with the greatest anti-cancer activity at the dosage used. In especially preferred composition embodiments the anti-cancer activity of a compound(s) of Formula [X] and a fatty acid(s) synergize.

A disclosure embodiment is a pharmaceutical composition comprising a therapeutically effective amount of a compound(s) of Formula [X] and 9Z,11E conjugated linoleic acid, optionally in a 1:1 ratio.

### Prodrugs

In addition, compounds of the Formulas [X] may have prodrug forms. Any compound that will be converted or react under biological conditions, e.g. in vivo, to provide the bioactive agent (i.e., a compound of Formula [X]) is a prodrug within the scope and spirit of this disclosure. For example, a derivative of a compound of Formula [X] that can hydrolyze, oxidize, or otherwise react under biological conditions to provide a compound of Formula [X]. For example, prodrug compounds of Formula [X] may be carboxylate ester moieties. A carboxylate ester may be conveniently formed by esterifying any of the carboxylic acid functionalities found on the disclosed structure(s). For example, prodrug compounds of Formula [X] comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of a compound of Formula [X] that comprise —NO, —NO₂, —ONO, or —ONO₂ moieties. Various forms of prodrugs are well known in the art. For introductory teaching in this area, see:
a) Design of Prodrugs, edited by H. Bundgaard, (Elsevier, 1985), and Methods in Enzymology, Vol. 42, p. 309-396, edited by K. Widder, et. al. (Academic Press, 1985);
b) A Textbook of Drug Design and Development, edited by Krosgaard-Larsen and H. Bundgaard, Chapter 5, “Design and Application of Prodrugs,” by H. Bundgaard, p. 113-191(1991);
c) H. Bundgaard, Advanced Drug Delivery Reviews, Vol. 8, p. 1-38(1992);
d) H. Bundgaard, et al., Journal of Pharmaceutical Sciences, Vol. 77, p. 285(1988); and
e) N. Kakeya, et. al., Chem Phar Bull, Vol. 32, p. 692(1984).
f) Burger's Medicinal Chemistry and Drug Discovery, 172-178, 949-982 (Manfred E. Wolff ed., 5^{th} ed. 1995)
g) Medicinal Chemistry: Principles and Practice, King, F. D., ed. The Royal Society of Chemistry, Cambridge, UK (1994); Testa, B. et al., Hydrolysis in Drug and Prodrug Metabolism. Chemistry, Biochemistry and Enzymology, VCHA and Wiley-VCH, Zurich, Switzerland (2003); The Practice of Medicinal Chemistry, Wermuth, C. G., ed., Academic Press, San Diego, Calif. (1999); Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association); Prodrugs: Challenges and Reward, edited by Valentino Stella, Ronald Borchardt, Michael Hageman, Reza Oliyai, Hans Maag, Jefferson Tilley, (Springer, 2007).

Prodrugs include compounds of this disclosure wherein a hydroxy, amino or mercapto group is bonded to any group that, when the prodrug of the compound of this disclosure is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of this disclosure and the like.

### Dosage

As used herein, the term “therapeutically effective amount” or “effective amount” refers to the amount of an administered compound sufficient to effect a beneficial or desired result(s) in the subject, e.g. prevents, reduces or eliminates a cause and/or symptom(s) of a disease/disorder, optionally in combination with other active compound(s). An effective amount can be administered in one or more administrations, applications or dosages and is not intended to be limited to a particular formulation or administration route.

The effective amount of a compound of the present disclosure may be determined and by one of ordinary skill in the art. The specific dose level and frequency of dosage for any particular subject may vary and will depend upon a variety of factors, including the activity of the specific compound employed, the metabolic stability and length of action of that compound, the pharmacokinetics of the compound, the formulation of compound used, the species, age, body weight, general health, medical condition, medical history, resilience, sex and diet of the subject, the mode/route and frequency of administration, rate of excretion, renal and hepatic function of patient, drug combination, concurrent treatment and type and severity/extent of the particular condition, nature/type/extent of presenting symptoms, the desired effect/outcome and/or the responsiveness/reaction of the subject. In an embodiment, a physician or veterinarian, optionally using their professional judgement, determines and prescribes the effective amount of the drug required. Broadly, small dosages may be used initially and, if necessary, increased by small increments until the desired effect under the circumstances is reached. The skilled practitioner is able to determine the appropriate dose based on factors disclosed herein and the effective dose derived from animal and clinical studies. Alternatively, or in addition, it may be determined empirically using known testing protocol(s) or by extrapolation from in vivo and/or in vitro test and/or diagnostic data. One skilled in the art using routine methods can determine effective dosage levels; that is, the dosage levels necessary to achieve the desired result. Typically, human clinical applications of products are commenced at lower dosage levels, with dosage level being increased until the desired effect is achieved. It is further understood that for any particular individual subject, specific dosage regimen can be adjusted over time according to the individual need and the professional judgment of the person(s) administering or supervising the administration of the compound(s)/composition(s).

An exemplary effective amount of compounds of Formula [X] may be within the dosage range of about 0.001 to about 300 mg/kg, preferably about 0.2 to about 50 mg/kg and more preferably about 0.5 to about 25 mg/kg (or from about 1 to about 2500 mg, preferably from about 5 to about 2000 mg), or from 1 microgram to 100 milligram per kilogram body weight per day, on a regimen in single or 2 to 6 (or more) divided daily doses (optionally administered at a specified interval), or weekly, monthly, yearly, once every 2 to 20 years, or only once, or administered in a continuous infusion for a period (all of the specific dosages which lie between the upper and lower dosages stated above are contemplated in the present disclosure). But more exactly it depends upon the compound used, the condition and its advancement/severity (e.g. the type and grade of cancer), the route of administration, type of dosing (e.g. pulse or consistent etc.), what other treatments are undertaken alongside or previously (e.g. chemotherapeutics, surgery, radiotherapy, immunotherapy etc.), the age, sex, condition, previous/other diseases of the patient, pharmacokinetics of compound in that patient, response to treatment and exceptions to this dosage range are contemplated by the present disclosure, and can be changed during treatment to find the optimum. Optimal dosage to be administered to a subject can be determined by those skilled in the art. Persons of ordinary skill in the art can estimate repetition rates for dosing based, in total or in part, on measured residence times and concentrations of the drug in bodily fluids or tissues. Clinical trials may be used to optimize the dose and dosing frequency for any particular compound, with subsequent further optimization for each particular subject by the direction of one of ordinary skill of the art e.g. a medical/veterinary practitioner. When the compounds described herein are co-administered with another agent, the effective amount may be less than when the agent is used alone.

Once improvement of the subject's disease/disorder/condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Subject may, however, require intermittent treatment on a long-term basis upon any recurrence of symptoms. Subjects may also require chronic treatment on a long-term basis. The case wherein the subject's condition does not improve, upon the (e.g.) doctor's discretion the administration of the compounds may be administered chronically, that is, for an extended period of time, including throughout the duration of the subject's life in order to ameliorate or otherwise control or limit the symptoms of the subject's disease or condition.

In some embodiments, a human dose(s) can be extrapolated from an animal study/studies (e.g. refer Fingle and Woodbury, Chapter 1 in Goodman and Gilman's The Pharmacological Basis of Therapeutics, 5^{th} Ed., MacMillan Publishing Co., New York (1975), pages 1-46).

### Pharmaceutical Composition

While it is possible for a compound of the present disclosure to be administered alone, it is preferable to administer the compound as a pharmaceutical formulation/composition. The compound(s) of Formula [X], or an enantiomer or a mixture of enantiomers thereof; or a pharmaceutically acceptable salt, solvate, hydrate, prodrug, co-crystal, clathrate, or polymorph thereof, may be formulated, alone or together, in suitable dosage unit with pharmaceutically acceptable excipients, carriers, adjuvants and vehicles, appropriate for each route of administration.

The term “pharmaceutical composition” means a composition comprising a compound of this disclosure in combination with at least one additional pharmaceutically acceptable carrier, inert or active, making the composition especially suitable for diagnostic or therapeutic use in vivo. Disclosed is a pharmaceutical composition of a therapeutically effective amount of a compound(s) of Formula [X] or a pharmaceutically acceptable salt thereof, and one or more pharmaceutically acceptable carriers, additives and/or diluents. This can be manufactured by a method of the art including, but not restricted to, mixing, dissolving, granulating, dragee-making, levigating, emulsifying, encapsulating, entrapping, melt-spinning, spray-drying, or lyophilizing processes. Pharmaceutical compositions may be presented in unit dose forms containing a predetermined amount of active ingredient, e.g. a compound(s) of Formula [X], per unit dose.

A “pharmaceutically acceptable carrier” refers to media generally accepted in the art for the delivery of biologically active agents to animals, in particular, mammals, including (without limitation), i.e., adjuvant, excipient, carrier or vehicle, such as diluents, preserving agents, preservatives, stabalizers, fillers, flow regulating agents, disintegrating agents, encapsulating materials, coating agents, release agents, wetting agents, emulsifying agents, water, phosphate buffered saline solution, emulsions (e.g., such as an oil/water or water/oil emulsions), suspending agents, anti-oxidants, buffers, pH buffers, tonicity adjusters, osmolality adjusting agents, physiological substances, pharmacological substances, sweetening agents, flavoring agents, coloring agents, perfuming agents, bulking agents, antibacterial agents, antifungal agents, surfactants, humectants, absorbents, precipitation inhibitors, adsorbants, solution retarding agents, absorption delaying agents, solvents, anti-foaming agents, salivary stimulating agents, absorption accelerators, cooling agents, lubricating agents, viscosity enhancing agents, dispersion media, dispensing agents, and the like, depending on the nature of the mode of administration and dosage forms. Most preferably, but not restrictively, the chosen pharmaceutically acceptable vehicle(s) (e.g., carrier(s), adjuvant(s), and/or other excipient(s)) has met the required standards of toxicological and manufacturing testing and/or is included on the Inactive Ingredient Guide prepared by the U.S. Food and Drug administration. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 18^{th} Ed. (1990) or Remington: The Science and Practice of Pharmacy, 22^{nd} Ed., Pharmaceutical Press (2013) or Introduction to Pharmaceutical Dosage Forms, 4^{th} ed., Lea & Febiger, Philadelphia (1985) or Handbook of Pharmaceutical Excipients (Raymond C. Rowe et al., APhA Publications, 4^{th} edition 2003) or Goodman & Gilman's The Pharmacological Basis of Therapeutics (Joel G. Hardman et al., eds., McGraw Hill Professional, 10^{th} ed. 2001, or 13^{th} ed. 2017) or Pharmaceutical Dosage Forms and Drug Delivery Systems (Howard C. Ansel et al., eds., Lippincott Williams & Wilkins Publishers, 7^{th} ed. 1999), Pharmaceutical Preformulation and Formulation (Gibson Ed., CRC Press LLC: Boca Raton, Fla., 2004), “Oral Lipid-Based Formulations: Enhancing the Bioavailability of Poorly Water-Soluble Drugs”, David J. Hauss, Informa Healthcare, 2007; and “Role of Lipid Excipients in Modifying Oral and Parenteral Drug Delivery: Basic Principles and Biological Examples” Kishor M. Wasan, ed. Wiley-Interscience, 2006. Suitable pharmaceutical compositions may be formulated by means known in the art, and their mode of administration and dose determined by the skilled practitioner. These protocols are routine procedures and any modifications are well within the scope of one skilled in the art and from the teaching herein. In preferred embodiments, pharmaceutical compositions according to this disclosure are sterile compositions. Except insofar as any conventional media or agent is incompatible with the active ingredient(s), its use in the therapeutic composition is contemplated. Supplementary active ingredient(s) can also be incorporated into the composition.

A therapeutic compound disclosed herein may be formulated by itself in a pharmaceutical composition, or may be formulated together with one or more other therapeutic compounds disclosed herein in a single pharmaceutical composition. Depending upon the particular condition, or disease, to be treated, additional therapeutic agent(s) that are normally administered to treat that condition may also be present in the compositions disclosed herein.

Some examples of materials which can serve as pharmaceutically acceptable carriers include: (1) sugars, such as lactose, glucose and sucrose; (2) starches, such as corn starch and potato starch; (3) cellulose, and its analogs, such as sodium carboxymethyl cellulose, ethyl cellulose and cellulose acetate; (4) powdered tragacanth; (5) malt, (6) gelatin; (7) talc.; (8) excipients, such as cocoa butter and suppository waxes; (9) oils, such as peanut oil, cotton seed oil, safflower oil, sesame oil, olive oil, corn oil and soybean oil: (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroide and aluminum hydroxide; (15) alginic acid, (16) pyrogen-free water, (17) isotonic saline; (18) Ringer's solution; (19) ethyl alcohol; (20) phosphate buffer solutions; and (21) other non-toxic compatible substances employed in pharmaceutical formulations.

Buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; (3) metal chelating agents, such as citric acid, ethylenediamine tetraacetic acid (EDTA), sorbitol, tartaric acid, phosphoric acid, and the like. Useful preservatives include, without limitation, benzalkonium chloride, chlorobutanol, thimerosal, phenylmercuric acetate, phenylmercuric nitrate, a stabilized oxychloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor.

In certain embodiments, the pharmaceutical preparation is non-pyrogenic i.e. does not substantially elevate the body temperature of a subject.

The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound(s) of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

### Cosmetic Composition

At all places herein that a “pharmaceutical composition” is referred to, in alternative embodiments this is substituted with “cosmetic composition”, wherein some good starting guides in this area: “Harry's Cosmeticology” [e.g. its 9^{th} edition by Meyer R. Rosen (Editor)], Chemical Publishing Company, USA, and Milady's “Standard Textbook of Cosmetology” (Delmar Learning), and “Formulation Technology. Emulsions, Suspensions, Solid Forms” by Hans Mollet, Arnold Grubenmann and Helen Payne, published by John Wiley & Sons, and “Chemistry and Technology of the Cosmetics and Toiletries Industry” by Clifford Williams Schmitt, Kluwer Academic Publishers, and Fiedler's “Encyclopedia of Excipients”, Cantor Verlag Aulendorf. In further embodiments, this is substituted with “supplement composition” or “dietary supplement composition” or “nutraceutical composition” or “cosmaceutical composition” or similar. At all places herein that “pharmaceutically-acceptable” or “pharmaceutically acceptable” is referred to, in alternative embodiments this is substituted with “cosmetically-acceptable”.

### Administration

A compound(s) of Formula [X] may be administered by any means suitable for the condition to be treated e.g. orally (e.g. taken with fatty food(s) to help absorption), topically, mucosally, locally, by inhalation, by intubation, by injection (e.g. injection(s) into a joint(s) e.g. the knee(s) and/or elbow(s) and/or wrist(s) and/or shoulder(s) and/or ankle(s) and/or hip(s) and/or one or more joints of the hand(s) and/or foot/feet), by infusion, by continuous infusion, by localized perfusion, via a catheter, via a lavage, parenteral administration. e.g. by intramuscular, intradermal, epicutaneous, intraperitoneal, intravenous or intra-arterial/intraarterial (infusion or bolus, dose given continuously or intermittently), intracisternal, epidural, intrasternal, intramedullary, intravesical injection or infusion, direct injection/infusion into a ventricle of the brain, administration via the cerebrospinal fluid (CSF, e.g. by lumbar injection and/or injection into the cisterna magna), intracranial, stereotaxic microinjection, instillation, subcutaneous injection/implant, transdermal (e.g. by skin patch, controlled release patch), transmucosal (e.g., nasal, sublingual, vaginal, buccal, or rectal) or oral routes, all using dosage forms well known to those of ordinary skill in the pharmaceutical arts. For further example: oral, enteric, parenteral, enteral (e.g. oral, buccal, sublabial, lingual, sublingual), infusion, injection, transdermal, intradermal, topical, paste applied to tongue, intravaginal (e.g. pessaries, tampons), intrarectal, rectal (e.g. by suppository, retention enema, enema, Murphy drip), transcutaneous, intracutaneous, subcutaneous, sublabial, subcuticular, intramuscular, intraarticular, intracapsular, subcapsular, subarachnoid, ocular/intraocular/ophthalmic (e.g. by eye drops {optionally using a measured dose eyedropper}, eye lotion, thin film), intraorbital, intraocular, intravitreal, retrobulbar, subconjunctival, aural, ear drops, intrathecal, intraventricular, intracardiac, intrapericardial, intrasynovial, intracerebral, intracranial, intracerebroventricular (optionally using a Ommaya reservoir), intraduodenal, intradermal, transdermal, intrapleural, intraspinal, intrasternal, intrathecal, intralesional, intratumoral, intracavernous (base of penis), intraprostatically, extra-amniotic administration/infusion, intraumbilical, perivascular administration, intravesicular, intraosseously (e.g. intraosseous infusion), endotracheally, transtracheal, intratracheal, intravaginal, nasally, intranasal, by inhalation, insufflation (snorting), by skin patch, by dermal patch, by transdermal patch (e.g., without restriction, matrix type patch, reservoir type patch, monolithic drug-in-adhesive type patch, multilaminate drug-in-adhesive type patch, and the like, refer Ghosh TK, Pfister WR, Yum SI, Transdermal and Topical Drug Delivery Systems, Interpharm Press, Inc., one of ordinary skill in the art can determine other patches which can be employed in the present disclosure), by adhesive bandage, transdermal spray, transdermal implant, nanocell injection, patient controlled pump, peripherally inserted central catheter (PIC line), delivery by catheter, indwelling catheter, gastric/duodenal feeding tube, gastrostomy, enteral nutrition, balloon (drug-eluting balloon), stent (drug-eluting stent), vaginal ring, vaginal sponge, vaginal douche, intrauterine device, cervical ring and the like, local injection into a body part(s) that is affected by a cancer(s), intratumorally. The compound(s) may be delivered by pill/tablet/capsule/pastille/time release technology/modified release dosage/osmotic delivery system/orally disintegrating tablet/film/thin-film/oral thin film (including multi-layer film)/dissolving film/oral drug strip/lollipop/sublingual drops/lozenges/effervescent buccal tablet/chewing gum/smoking device/dry-powder inhaler/vaporizer/nebulizer/metered-dose inhaler/gas mask/nasal cannula/nasal spray/mucoadhesive microdisc/pessary/suppository/electuary/emulsion/extended-release syrup/effervescent [powder/tablet]/hydrogel/molecular encapsulation/powder/softgel/solution/suspension/slurry/liquid/syrup/syrup concentrate for dilution and/or addition of carbonated water/tincture/mouthwash/toothpaste/ointment/oral spray/nasal spray/liniment/electrophoretic dermal delivery system/liposomes/transfersome vesicles/lip balm/shampoo/jet injector/vapor/solid/decoction/ointment/cream/salve/oil/rinse/alcohol/spray/aerosol/foam/paste/lotion/tincture/shake lotion/gel/implant/dressing/sponge/tape/drop/powder/patch/transdermal patch/electroporation/iontophoresis/phonophoresis/sonophoresis, penetration enhancers may be incorporated e.g. see Finnin and Morgan, J. Pharm. Sci. 1999, 88, 955-958). In other embodiments, local administration may be accomplished by implanting a sustained-release device such as a pump or a micropump, or sustained-release implant, such as a bead or gel that contains the compound(s), e.g. anti-cancer agent, and slowly releases the drug into the desired area over time. Suitable devices for parenteral administration include needle (including microneedle) injectors, needle-free injectors and infusion techniques. The compound may be delivered orally, such as in the form of tablets (optionally chewable)/caplets/capsules/pills/lozenges (including liquid filled)/pastilles/electuary/paste/oral films/[buccal/mucoadhesive] patches (each of which also includes sustained release or timed release formulations), granules, pastilles (e.g. using an inert base, such as gelatin and glycerin, or sucrose and acacia), lozenges (e.g. using a flavored basis, usually sucrose and acacia or tragacanth), lyophilisates, dragees, cachets, troches, microgranules, pellets, soft-gels, powders, powders for reconstitution, dispersions, tinctures, or liquid formulations including syrups, liquids, solutions, elixirs, suspensions (e.g., aqueous or non-aqueous liquid suspensions, oil-in-water emulsions, or a water-in-oil liquid emulsions), emulsions, microemulsions, solutions, mouthwashes, elixirs and the like, each containing a predetermined amount of a compound(s) of the present disclosure as an active ingredient; liquid dosage forms suitable for parenteral administration to a patient; eye drops or other ophthalmic preparations suitable for topical administration; emulsions or magmas; sublingually; bucally; transdermally; parenterally, such as by subcutaneous, intravenous (bolus and/or infusion), intramuscular or intrasternal injection or infusion (e.g., as sterile injectable aqueous or non-aqueous solutions or suspensions; sterile solids {e.g., crystalline or amorphous solids} that can be reconstituted to provide liquid dosage forms suitable for parenteral administration); nasally such as by inhalation spray (aerosol, nasal spray, inhaler, nebuliser etc.); rectally such as in the form of suppositories; or liposomally; each containing a predetermined amount of a compound(s) of the present disclosure as an active ingredient. A compound(s) of the present disclosure may also be administered as a bolus, electuary or paste. A compound(s) of this disclosure can be administered alone, but generally will be administered with a pharmaceutical carrier selected on the basis of the chosen route of administration and standard pharmaceutical practice. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

To illustrate, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

In solid dosage forms of this disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Exemplary compositions for oral administration include suspensions which may contain, for example, microcrystal line cellulose for imparting bulk, alginic acid or sodium alginate as a suspending agent, methylcellulose as a viscosity enhancer, and sweeteners or flavouring agents such as those known in the art; and immediate release tablets which may contain, for example, microcrystalline cellulose, sugar(s), dicalcium phosphate, starch, magnesium stearate and/or lactose and/or other excipients, binders, extenders, disintegrants, diluents, granulating agents and lubricants such as those known in the art. The compounds may be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavours, fragrances, colorants, colouring agents and stabilizers may also be added for ease of fabrication and use.

The pharmaceutical compositions disclosed herein for (e.g. oral) administration may be also administered in the forms of liposomes, liposome suspensions, niosomes, micelles, micellar solutions, microspheres, or nanosystems. Micellar dosage forms can be prepared as described in U.S. Pat. No. 6,350,458.

Liquid dosage forms for oral administration of the compounds of this disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as preservatives, buffers, propellants, suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents. Indeed, liquid dosage forms for oral administration can contain coloring and flavoring to increase patient acceptance.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Oily Suspensions may be formulated by suspending a therapeutic compound disclosed herein in admixture with (a) Vegetable oils, such as, e.g., almond oil, arachis oil, avocado oil, canola oil, castor oil, coconut oil, corn oil, cottonseed oil, grape seed oil, hazelnut oil, hemp oil, linseed oil, olive oil, palm oil, peanut oil, rapeseed oil, rice bran oil, safflower oil, sesame oil, soybean oil, soya oil, sunflower oil, walnut oil, wheat germ oil, or a combination thereof, (b) a saturated fatty acid, an unsaturated fatty acid, or a combination thereof. Such as, e.g., palmitic acid, stearic acid, oleic acid, linoleic acid, linolenic acid, or a combination thereof, (c) mineral oil such as, e.g., liquid paraffin, (d) Surfactants or detergents. The oily suspensions may contain a thickening agent, for example beeswax, hard paraffin or cetyl alcohol. Sweetening agents and flavoring agents may be added to provide a palatable oral preparation. These compositions may be preserved by the addition of an antioxidant such as ascorbic acid.

A therapeutic compound disclosed herein may be in the form of oil-in-water emulsions. The oily phase may be a vegetable oil as disclosed herein or a mineral oil as disclosed herein or mixtures thereof. Suitable emulsifying agents may be naturally occurring gums, such as, e.g., gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Pharmaceutical compositions of this disclosure suitable for parenteral administration comprise one or more compounds of this disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Inhibition of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Exemplary compositions for parenteral administration include injectable solutions or suspensions, preferably sterile and preferably buffered to an appropriate pH and isotonicity, and/or which may contain, for example, suitable non-toxic, parenterally acceptable diluents, oils or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, ethyl alcohol, polyethylene glycol, polypropylene glycol, corn oil, cottonseed oil, peanut oil, sesame oil, ethyl oleate, isopropyl myristate, and benzyl benzoate. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Compounds that increase the solubility of one or more of the active ingredients disclosed herein can also be incorporated into the parenteral dosage forms provided herein. For example, cyclodextrin and/or its derivatives can be used to increase the solubility of a compound provided herein.

Sterile compositions of compound(s) of this disclosure are contemplated by this disclosure, indeed preferred, including compositions that are in accord with national and local regulations governing such compositions.

Formulations of the pharmaceutical compositions of this disclosure for rectal or vaginal administration may be presented as a suppository, which may be prepared by mixing one or more compounds of this disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active ingredient. Exemplary compositions for rectal/vaginal administration include suppositories which may contain, for example, suitable non-irritating excipients, such as cocoa butter, synthetic glyceride esters or polyethylene glycols, which are solid at ordinary temperatures but liquefy and/or dissolve in the rectal/vaginal cavity to release the drug. Formulations of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this disclosure, excipients, such as animal and vegetable fats, oils, waxes, paraffins, starch, tragacanth, cellulose analogs, polyethylene glycols, silicones, bentonites, silicic acid, talc and zinc oxide, or mixtures thereof.

Powders and sprays can contain, in addition to a compound of this disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ophthalmic formulations, eye ointments, powders, solutions, drops and the like, are also contemplated as being within the scope of this disclosure.

Transdermal patches have the added advantage of providing controlled delivery of a compound of the present disclosure to the body. Such dosage forms can be made by dissolving or dispersing the composition in the proper medium. Absorption enhancers can also be used to increase the flux of the composition across the skin. The rate of such flux can be controlled by either providing a rate-controlling membrane or dispersing the compound in a polymer matrix or gel.

In some embodiments, a compound or pharmaceutical formulation in accordance may be administered by means of a medical device or appliance such as an implant, graft, prosthesis, stent etc. Implants may be devised which are intended to contain and release such compounds or compositions. An example would be an implant made of a polymeric material adapted to release the compound over a period of time.

A compound(s) of the present disclosure can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. Any non-toxic, physiologically acceptable and metabolizable lipid capable of forming liposomes can be used. A compound(s) described herein, when in liposome form can contain, in addition to a compound(s) described herein, stabilizers, preservatives, excipients, and the like. Methods to form liposomes are known in the art. (See, e.g., Prescott, Ed., Methods in Cell Biology, Volume XIV, Academic Press, New York, N.Y., (1976), p 33 et seq.). See e.g. Liposomes as drug carriers: Recent trends and progress. Edited by Gregory Gregoriadis. John Wiley: Chichester, UK. An active agent(s) disclosed herein can also be formulated as liposomes. Liposomes are prepared by methods known in the art, such as described in Epstein et al., Proc. Natl. Acad. Sci. USA, 82:3688 (1985): Hwang et al., Proc. Natl Acad. Sci. USA, 77:4030 (1980); and U.S. Pat. Nos. 4,485,045 and 4,544.545. Liposomes with enhanced circulation time are disclosed in U.S. Pat. No. 5,013,556.

Compounds of the present disclosure may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present disclosure may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Consumable oral films for human or veterinary use are typically pliable water-soluble or water-swellable thin film dosage forms which may be rapidly dissolving or mucoadhesive and typically comprise a compound(s) of Formula [X], a film-forming polymer, a binder, a solvent, a humectant, a plasticizer, a stabilizer or emulsifier, a viscosity-modifying agent, a solvent and, more optionally, a saliva stimulating agent and one or more agents relating to taste. Alternatively, the compound(s) of Formula [X] may be in the form of multiparticulate beads.

The compounds of this disclosure (including pharmaceutically acceptable salts thereof) can be administered intranasally or by inhalation, in the form of a dry powder, from a dry powder inhaler, as an aerosol spray from a pressurized container, pump, spray, atomizer (for example an atomizer using electrohydrodynamics to produce a fine mist), or nebulizer, or as nasal drops. In inhaled dosage forms, the therapeutic compound(s) may be prepared for delivery as an aerosol in a liquid propellant for use in a pressurised (PDI) or other metered dose inhaler (MDI). A therapeutic compound may also be delivered using a nebuliser or other aerosol delivery system. The pharmaceutical composition may be in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1.1.1.2.3,3,3-heptafluoropropane.

### Targeted Delivery

A pharmaceutical composition disclosed herein may also be formulated to be targeted to a particular tissue, receptor, or other area of the body of the subject to be treated, including liposome-, resealed erythrocyte-, and antibody based delivery systems. Examples include, but are not limited to, U.S. Pat. Nos. 6,316,652; 6,274,552; 6,271,359; 6,253,872:6,139,865; 6,131.570; 6,120,751; 6,071,495; 6,060,082:6,048,736; 6,039,975; 6,004,534; 5,985,307; 5,972,366; 5,900,252; 5,840,674; 5,759,542; and 5,709,874.

### Topical Administration

Certain compounds disclosed herein may be administered topically, that is by non-systemic administration. This includes the application of a compound(s)/composition(s) disclosed herein externally to the epidermis or the buccal cavity and the instillation of such a compound into the ear, eye and nose, such that the compound does not significantly enter the blood stream.

A pharmaceutical composition(s) disclosed herein may be administered topically to the skin, orifice(s), or mucosa. The topical administration, as used herein, include (intra) dermal, conjuctival, intracorneal, intraocular, ophthalmic, auricular, transdermal, nasal, vaginal, uretheral, respiratory, and rectal administration.

The pharmaceutical compositions disclosed herein may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions disclosed herein may also comprise liposomes, niosomes, micelles, microspheres, nanosystems, and mixtures thereof.

Pharmaceutically acceptable carriers and excipients suitable for use in the topical formulations disclosed herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions may also be administered topically by electroporation, iontophoresis, phonophoresis, sonophoresis and microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions disclosed herein may be disclosed in the forms of ointments, creams, and gels. Suitable ointment vehicles include oleaginous or hydrocarbon vehicles, including such as lard, benzoinated lard, olive oil, cottonseed oil, and other oils, white petrolatum; emulsifiable or absorption vehicles, such as hydrophilic petrolatum, hydroxy stearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington. The Science and Practice of Pharmacy, Supra). These vehicles are emollient but generally require addition of antioxidants and preservatives. Suitable cream base can be oil-in-water or water-in oil. Cream vehicles may be water-washable, and contain an oil phase, an emulsifier, and an aqueous phase. The oil phase is also called the “internal’ phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol R; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum, sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions disclosed herein may be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington. The Science and Prac tice of Pharmacy, Supra. Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions disclosed herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions disclosed herein may be administered ophthalmically in the forms of solutions, suspensions, ointments, emulsions, gel-forming solutions, powders for solutions, gels, ocular inserts, and implants.

The pharmaceutical compositions disclosed herein for topical administration may be formulated to be immediate-release or modified-release, including delayed-, sustained-, pulsed-, controlled-, targeted-, and programmed-release.

### Controlled Release

In certain embodiments, at least one active ingredient provided herein is administered by controlled release means or by a delivery device. Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500; 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated herein by reference in its entirety.

In certain embodiments, such dosage forms are be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

These compositions may also optionally contain opacifying agents and may be of a composition that they release the active ingredient(s) only, or preferentially, in a certain portion of the gastrointestinal tract, optionally, in a delayed manner

Modified release formulations include delayed-, extended-, prolonged-, sustained-, pulsed-, pulsatile-, controlled-, accelerated-, fast-, targeted- and programmed-release. And gastric retention dosage forms. Some suitable modified release formulations for the purposes of this disclosure are described in U.S. Pat. No. 6,106,864.

In some cases, in order to prolong the effect of a drug, it is desirable to slow the absorption of the drug. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of an administered drug form is accomplished by dissolving or suspending the drug in an oil vehicle or by forming and using microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides).

A therapeutic compound(s) disclosed herein, or a composition comprising such a therapeutic compound(s), may be incorporated into a drug delivery platform in order to achieve a controlled release profile over time. Such a drug delivery platform comprises a therapeutic compound(s) disclosed herein dispersed within a polymer matrix, typically a biodegradable, bioerodible, and/or bioresorbable polymer matrix. Examples of biodegradable, bioerodible, and/or bioresorbable polymers and methods useful to make a drug delivery platform are described in, e.g., Drost, et. al., Controlled Release Formulation, U.S. Pat. No. 4,756,911; Smith, et. al., Sustained Release Drug Delivery Devices, U.S. Pat. No. 5,378,475; Wong and Kochinke. Formulation for Controlled Release of Drugs by Combining Hyrophilic and Hydrophobic Agents, U.S. Pat. No. 7,048,946; Hughes, et. al., Compositions and Methods for Localized Therapy of the Eye, U.S. Patent Publication 2005/0181017: Hughes, Hypotensive Lipid-Containing Biodegradable Intraocular Implants and Related Methods, U.S. Patent Publication 2005/0244464; Altman, et al., Silk Fibroin Hydrogels and Uses Thereof, U.S. Patent Publication 2011/0008437; each of which is incorporated by reference in its entirety.

In aspects of this embodiment, a polymer composing the matrix is a polypeptide such as, e.g., silk fibroin, keratin, or collagen. In other aspects of this embodiment, a polymer composing the matrix is a polysaccharide such as, e.g., cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix is a polyester such as, e.g., D-lactic acid, L-lactic acid, racemic lactic acid, glycolic acid, caprolactone, and combinations thereof.

### Matrix Controlled Release Devices, Osmotic Controlled Release Devices, Multiparticulate Controlled Release Devices

A compound(s)/composition(s) of this disclosure can be administered to a subject by one or more of these devices, which are described, for example, in US2009/0202540A1, herein incorporated in its entirety, or a different patent application(s) previously/presently assigned to Auspex Pharmaceuticals Inc.

### Implants

Implantable devices containing a compound(s) of Formula [X] are also included in this disclosure, optionally a bioerodable implant comprising an the active agent dispersed within a biodegradable polymer matrix, optionally wherein 75% of the particles of the active agent have a diameter of less than about 10 um. This disclosure provides implantable (optionally rechargeable or biodegradable) devices that contain a compound(s) of Formula [X], or a pharmaceutically acceptable salt, solvate, hydrate or prodrug thereof. A variety of biocompatible polymers (including hydrogels), including both biodegradable and non-degradable polymers, can be used to form an implant for the sustained release of a drug at a particular target site. The biodegradable polymer can be, for example, a poly (lactic-co-glycolic) acid (PLGA) copolymer.

Description of some example implantable devices can be found, for example, in U.S. Publication Nos. 2004/0009222, 2004/0180075, 2005/0048099, 2005/0064010 and 2005/0025810, the contents of which are incorporated herein by reference.

### Micronization

Micronization is the process of reducing the average diameter of a material's particles. It usually refers to the reduction of average particle diameters to the micrometer range, but can also describe further reduction to the nanometer scale (nanonization). A disclosure embodiment is a micronized/nanonized form of a compound(s) and/or composition(s) and/or formulation(s) of Formula [X]. A pharmaceutical composition comprising a micronized/nanonized compound(s) of Formula [X] or a pharmaceutically acceptable salt, solvate, hydrate or prodrug thereof, wherein the compound/composition particles have an average diameter of less than 30 (or 10 or 5) microns (or 400 nm). Smaller particles have increased surface area to volume ratio, which can deliver greater water solubility in the gastrointestinal tract and thence increased bioavailability, especially useful for compounds with poor water solubility, wherein many compounds of Formula [X] have poor water solubility. In certain embodiments, a large proportion of the particles have diameters within a defined range. Drugs according to this disclosure can be micronized using conventional micronization equipment, such as the Micron-Master line of micronizers available from The Jet Pulverizer Company (Moorestown, N.J.), or processed by a third-party micronization processor such as Micron Technologies (Exton, Pa.). U.S. Pat. Nos. 6,645,466, 6,623,760, 6,555,135, hereby incorporated by reference.

### Nanoparticles

An aspect of the present disclosure provides a composition that includes nanoparticles comprising a compound(s) of Formula [X], or a pharmaceutically acceptable salt, solvate, hydrate or prodrug thereof. Such particles can, for example, have a mean diameter of 5 nm to 900 nm, or 50 nm to 500 nm, such as 100 nm to 200 nm. The term “nanoparticles” can refer to particles formed by a polymeric matrix in which the active compound is dispersed, also known as “nanospheres, and also refers to nanoparticles which are composed of a core containing the active compound which is surrounded by a polymeric membrane, also known as “nanocapsules. A description on preparing nanoparticles can be found, for example, in U.S. Pat. No. 6,264,922, incorporated herein by reference.

### Liposomes

Liposomes are a further drug delivery system. Accordingly, in the method of disclosure the active compound(s) can also be administered in the form of a liposome delivery system. Liposomes can be formed from a variety of phospholipids, such as cholesterol, stearylamine of phosphatidylcholines, and optionally incorporate one or more “shielding” moities. Liposomes being usable for the method of disclosure encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent, for example, a coating of hydrophilic polymer chains such as polyethyleneglycol (PEG) to extend the blood circulation lifetime of the liposomes. Such surface-modified liposomes are commonly referred to as “long circulating or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic. D. and Martin, F., eds., Boca Raton, Fla., (1995)), and the cited references therein. The pharmacokinetics exhibited by such liposomes are characterized by significantly prolonged blood circulation time, as compared to non surface-modified liposomes, which tend to be rapidly removed from the blood and accumulated in the liver and spleen.

The PEG moiety can have a molecular weight of for example, 750-20,000 Daltons, such as 1000-10,000 Daltons, in particular 2000-5000 Daltons. In one embodiment, the complex may comprise more than one type of PEG moiety (for example, PEG molecular weight 5K and PEG molecular weight 2K). The PEG moiety may further comprise a suitable functional group, such as, for example, methoxy, N-hydroxyl succinimide (NHS), carbodimide, etc., for ease of conjugating PEG to the lipid or to the targeting factor. Table 2 of Harasym et al. Advanced Drug Delivery Reviews 32:99-118(1998) provides examples of suitable functional groups. Functionalized PEG moieties can be purchased from, for example, Shearwater Polymer Inc. (Huntsville, Ala.) and Avanti Polar Lipid Inc. (Alabaster, Ala.). In an exemplary embodiment, the PEG moiety is N-[methoxy (polyethylene glycol)-5K] (PEG5k). Other types of hydrophilic polymers may be substituted for the PEG moiety, including, for example, poloxamer and poloxamine, as described in Feldman et al. (1997) Gene Therapy 4(3): 189-198: Lemieux et al. (2000) Gene Therapy 7(11): 986-91; Moghimi et al. (2000) Trends. In Biotechnology 18:412-420; Torchilin (1998) Journal of Microencapsulation 15(1): 1-19; and Claesson et al. (1996) Colloids & Surfaces A-Physicochemical & Engineering Aspects 112(2):-3, 131-139.

The PEG moiety may be conjugated to a suitable lipid to form a “pegylated lipid”. Preferably, the PEG moiety is covalently attached to the lipid. The PEG moiety can be conjugated to the lipid by methods known in the art. See, for example, Woodle (1998) Adv. Drug Delivery Reviews 32:139-152 and references cited therein; Haselgruber et al. (1995) Bioconjug Chem 6:242-248: Shahinian et al. (1995) Biochim Biophys Acta 1239:157-167; Zalipsky et al. (1994) FEBS Lett. 353:71-74; Zalipsky et al. (1997) Bioconjug Chem. 8(2): 111-118: Zalipsky et al. (1995) Bioconjug Chem. 6:705-708; Hansen et al. (1995) Biochim Biophys Acta. 1239(2): 133-44; Allen et al. (1995) Biochim Biophys Acta 1237(2): 99-108; Zalipsky (1995) Bioconjug Chem 6(2): 150-65; Zalipsky (1993) Bioconjug Chem 4(4): 296-9; and Zalipsky (1995) in Stealth Liposomes. (Eds: Lasic, D., et al.) CRC Press, Boca Raton, Fla., p. 93-102. Pegylated lipids are also available commercially from, for example, Shearwater Polymer Inc. (Huntsville, Ala.).

### Cyclodextrin

A composition comprising a cyclodextrin(s) and a compound(s) of Formula [X], or a pharmaceutically acceptable salt, solvate, hydrate or prodrug thereof, optionally wherein the composition is a liquid that comprises at least 30 mM of a compound of Formula [X], optionally wherein the cyclodextrin is a substituted cyclodextrin, optionally wherein the cyclodextrin is substituted on the 2-, 3- or 6-hydroxyl group of a glycopyranose moiety, optionally wherein the cyclodextrin is amorphous, optionally wherein the cyclodextrin is one or more of 2-hydroxypropyl-β-cyclodextrin (or derivative thereof)/hydroxypropyl-β-cyclodextrin (or derivative thereof)/β-cyclodextrin (or derivative thereof)/α-cyclodextrin (or derivative thereof)/γ-cyclodextrin (or derivative thereof), optionally wherein the composition is a lyophilized (e.g. water soluble), optionally wherein the cyclodextrin(s) is of the structure:

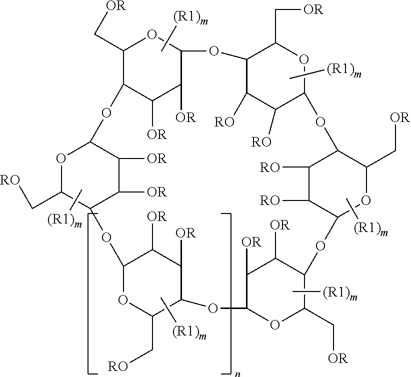

or a pharmaceutically acceptable salt, ester, solvate, or hydrate thereof. Wherein, each R is independently H, D, halogen, alkyl, alkenyl, alkynyl, cycloalkyl, heterocycloalkyl, aryl, or heteroaryl, each of which is optionally substituted; or C(O)OR^{B}, —OC(O)R^{B}, —C(O)R^{B}, or C (O)NR^{A}R^{B};
each R¹ is selected independently from H, D, alkyl, alkenyl, alkynyl, cycloalkyl, aryl, heteroaryl, halogen, hydroxy, amino, CN, CF₃, N₃, NO₂, OR^{B}, SR^{B}, SOR^{B}, SO₂R^{B}, —N(R^{B})S(O₂), R^{B}, —N(R^{B})S(O²)NR^{A}R^{B}, NR^{A}R^{B}, —C(O)OR^{B}, OC(O)R^{B}, C(O)R^{B}, C (O)NR^{A}R^{B}, or N(R^{B})C(O)R^{B}; each of which is optionally substituted;
wherein each R^{A} is independently hydrogen, deuterium, alkyl, alkenyl, alkynyl, cycloalkyl, heterocycloalkyl, aryl, or heteroaryl, each of which is optionally substituted;
wherein each R^{B} is independently hydrogen, deuterium, alkyl, alkenyl, alkynyl, cycloalkyl, heterocycloalkyl, aryl, or heteroaryl, each of which is optionally substituted;
wherein nis 1, 2, 3, 4, 5, 6, 7, 8, 9, or 10;
wherein each m is independently 0, 1, 2, 3, 4, or 5.

A way to produce a formulation, particularly a solution, of a compound(s) of Formula [X] or a derivative thereof, is through the use of cyclodextrin. By cyclodextrin, it is meant α-, β or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

By amorphous cyclodextrin, it is meant non-crystalline mixtures of cyclodextrins wherein the mixture is prepared from α-, β-, or γ-cyclodextrin. In general the amorphous cyclodextrin is prepared by non-selective additions, especially alkylation of the desired cyclodextrin species. Reactions are carried out to yield mixtures containing a plurality of components thereby preventing crystallization of the cyclodextrin. Various alkylated and hydroxyalkyl cyclodextrins can be made and of course will vary, depending upon the starting species of cyclodextrin and the addition agent used. Among the amorphous cyclodextrins suitable for compositions according to this disclosure are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin. The substituted γ-cyclodextrins may also be suitable, including hydroxypropyl, hydroxyethyl, glucosyl, maltosyl and maltotriosyl derivatives of γ-cyclodextrin.

The cyclodextrin of the compositions according to this disclosure may be α-, β-, or γ-cyclodextrin. α-cyclodextrin contains six glucopyranose units; β-cyclodextrin contains seven glucopyranose units; and γ-cyclodextrin contains eight glucopyranose units. The molecule is believed to form a truncated cone having a core opening of 4.7-5.3 angstroms, 6.0-6.5 angstroms, and 7.5-8.3 angstroms in α-, β-, or γ-cyclodextrin respectively. The composition according to this disclosure may comprise a mixture of two or more of the α-, β-, or γ-cyclodextrins. Typically, however, the composition according to this disclosure will comprise only one of the α-, β-, or γ-cyclodextrins.

The unmodified α-, β-, or γ-cyclodextrins are less preferred in the compositions according to this disclosure because the unmodified forms tend to crystallize and are relatively less soluble in aqueous solutions. More preferred for the compositions according to this disclosure are the α-, β-, and γ-cyclodextrins that are chemically modified or substituted. Chemical substitution at the 2, 3 and 6 hydroxyl groups of the glucopyranose units of the cyclodextrin rings yields increases in solubility of the cyclodextrin compound.

Most preferred cyclodextrins in the compositions according to this disclosure are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to this disclosure are hydroxypropyl, hydroxyethyl, glucosyl, maltosyl and maltotriosyl derivatives of β-cyclodextrin, carboxyamidom ethyl-β-cyclodextrin, carboxymethyl-β-cyclodextrin, hydroxypropyl-β-cyclodextrin and diethylamino-β-cyclodextrin.

Importantly, if the aqueous solution comprising the therapeutic compound(s) and a cyclodextrin is to be administered parenterally, especially via the intravenous route, a cyclodextrin will be substantially free of pyrogenic contaminants. Various forms of cyclodextrin, such as forms of amorphous cyclodextrin, may be purchased from a number of vendors including Sigma-Aldrich, Inc. (St. Louis, Mo., USA). A method for the production of hydroxypropyl-β-cyclodextrin is disclosed in Pitha et al., U.S. Pat. No. 4,727,064 which is incorporated herein by reference.

To produce the formulations according to this disclosure, a pre-weighed amount of a cyclodextrin compound, which is substantially pyrogen free is placed in a suitable depyrogenated sterile container. Methods for depyrogenation of containers and closure components are well known to those skilled in the art and are fully described in the United States Pharmacopeia 23 (United States Pharmacopeial Convention, Rockville, Md. USA). Generally, depyrogenation is accomplished by exposing the objects to be depyrogenated to temperatures above 400 degrees Centigrade for a period of time sufficient to fully incinerate any organic matter. As measured in U.S.P. Bacterial Endotoxin Units, the formulation will contain no more than 10 Bacterial Endotoxin Units per gram of amorphous cyclodextrin. By substantially pyrogen free is meant that the cyclodextrin con tains less than 10 U.S.P. bacterial endotoxin units per gram using the U.S.P. method. Preferably, the cyclodextrin will contain between 0.1 and 5 U.S.P. bacterial endotoxin units per mg, under conditions specified in the United States Pharmacopeia 23.

Sufficient sterile water for injection is added to the substantially pyrogen free amorphous cyclodextrin until the desired concentration of the cyclodextrin is in solution. To this solution a pre-weighed amount of the therapeutic compound(s), optionally a compound(s) of Formula [X], is added with agitation and with additional standing if necessary until it dissolves.

The solution is then filtered through a sterile 0.22 micron filter into a sterile holding vessel and is subsequently filled in sterile depyrogenated vials and is capped. For products that will be stored for long periods of time, a pharmaceutically acceptable preservative may be added to the solution of therapeutic compound(s) and cyclodextrin prior to filtration, filling and capping or alternatively, may be added sterilely after filtration.

As discussed above, the present disclosure provides improved water soluble formulations of compound(s) of Formula [X] and methods of preparing and employing such formulations. The advantages of these water soluble formulations are that a drug is entrapped in cyclodextrin in dissolved form. These compositions can be delivered in the form by slow infusions or by bolus injection or by other parenteral or oral delivery routes.

Additional description of the use of cyclodextrin for solubilizing compounds can be found in US 2005/0026849, the contents of which are incorporated herein by reference.

In an embodiment, the therapeutic action of a cyclodextrin(s), or derivative(s) thereof, synergizes with the therapeutic action of one or more compounds of Formula [X], or a pharmaceutically acceptable salt, solvate, hydrate or prodrug thereof. For non-limiting example, cyclodextrin(s) can slow aging (delay the onset of an age associated phenotype in a cell) and a compound of Formula [X] can slow aging (delay the onset of an age associated phenotype in a cell) and when co-administered, optionally in a pharmaceutical composition, these anti-aging properties add/synergize. A method of treating or delaying the onset of geriatric aging of the human or animal body, tissue, or organ comprising administering a composition that includes one or more cyclodextrins and one or more compounds of Formula [X] to a subject. A method of preventing, treating or delaying the onset of a disease/condition associated with aging, and/or a lipofuscin associated disorder, by administering a composition that includes one or more of cyclodextrins and one or more compounds of Formula [X] to a subject.

### Fast Melt Formulations

Rapid disintegration facilitates delivery of the active material. Rapidly disintegrating or dissolving, e.g. oral, dosage forms comprising at least one compound of this disclosure are contemplated. Illustrative teaching upon how to synthesize such is in the patent output of manufacturers including Cima Labs, Fuisz Technologies Ltd., Prographarmn, R. P. Scherer, Yamanouchi-Shaklee, and McNeil-PPC, Inc.

### Animal Feed

Componentry to this disclosure is to add a compound(s) of Formula [X] to animal feed and/or drinking water/fluid. How such feed premixes and complete rations can be prepared and administered are described in reference books (such as “Applied Animal Nutrition”, W.H. Freedman and CO., San Francisco, U.S.A., 1969 or “Livestock Feeds and Feeding” O and B books, Corvallis, Oreg., U.S.A., 1977).

### Cycling Therapy

In certain embodiments, the prophylactic/therapeutic agents provided herein are cyclically administered to a subject. Cycling therapy involves the administration of an active agent for a period of time, followed by a rest for a period of time, wherein the rest period equals a day, days, weeks, or months without the compound(s) being administered, before the administration period starts again, and this cycle is repeated over a period of time, optionally wherein the length (and dosage) of the administration and/or rest periods can be modulated, optionally to find the best cycle for the subject. A variant is wherein the rest period isn't complete rest, but the compound(s) is administered at lower dosage. Cycling therapy can reduce the development of resistance to one or more of the therapies, avoid, or reduce the side effects of one of the therapies, and/or improves the efficacy of the treatment. In an embodiment, a cycle of administration with one or more compounds of Formula [X] is implemented completely out of phase, or with a degree of overlap, with a cycle of administration of one or more other compounds of Formula [X], or another drug approved for human use by the regulatory body applicable to the country that the subject resides e.g. the FDA applies for a subject in the USA.

### Optimising for Brain/CNS Administration

Delivery approaches can be used to deliver therapeutic agents to the brain whilst circumventing the blood-brain barrier. Some such approaches can utilize intrathecal injections, surgical implants (Ommaya, Cancer Drug Delivery, 1:169-178(1984) and U.S. Pat. No. 5,222,982), interstitial infusion (Bobo et al., Proc. Natl. Acad. Sci. U.S.A., 91:2076-2080(1994)), and the like. These strategies deliver an agent to the CNS by direct administration into the cerebrospinal fluid (CSF) or into the brain parenchyma (ECF).

Drug delivery to the central nervous system through the cerebrospinal fluid can be achieved, for example, by means of a subdurally implantable device the “Ommaya reservoir”. The drug is injected into the device and subsequently released into the cerebrospinal fluid surrounding the brain. It can be directed toward specific areas of exposed brain tissue which then adsorb the drug. This adsorption is limited since the drug does not travel freely. A modified device, whereby the reservoir is implanted in the abdominal cavity and the injected drug is transported by cerebrospinal fluid (taken from and returned to the spine) to the ventricular space of the brain, is used for agent administration. Through omega-3 derivatization, site-specific biomolecular complexes can overcome the limited adsorption and movement of therapeutic agents through brain tissue.

Another strategy to improve agent delivery to the CNS is by increasing the agent absorption (adsorption and transport) through the blood-brain barrier and the uptake of therapeutic agent by the cells (Broadwell, Acta Neuropathol., 79:117-128(1989); Pardridge et al., J. Pharmacol. Experim. Therapeutics, 255:893-899(1990); Banks et al., Progress in Brain Research, 91:139-148(1992); Pardridge, Fuel Homeostasis and the Nervous System, ed.: Vranic et al., Plenum Press, New York, 43-53(1991)). The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier, or by its covalent coupling to a peptide vector capable of transporting the agent through the blood-brain barrier. Peptide transport vectors are also known as blood-brain barrier permeabilizer compounds (U.S. Pat. No. 5,268,164). Site specific macromolecules with lipophilic characteristics useful for delivery to the brain are described in U.S. Pat. No. 6,005,004. Other examples (U.S. Pat. Nos. 4,701,521, and 4,847,240) describe a method of covalently bonding an agent to a cationic macromolecular carrier which enters into the cells at relatively higher rates. These patents teach enhancement in cellular uptake of bio-molecules into the cells when covalently bonded to cationic resins. U.S. Pat. No. 4,046,722 discloses anti-cancer drugs covalently bonded to cationic polymers for the purpose of directing them to cells bearing specific antigens. The polymeric carriers have molecular weights of about 5,000 to 500,000. Such polymeric carriers can be employed to deliver compounds described herein in a targeted manner.

### Kits

The present disclosure also encompasses an article of manufacture. As used herein, article of manufacture is intended to include, but not be limited to, kits and packages. The kit may comprise multiple internal containers to keep components separate. An article of manufacture of the present disclosure, comprises: (a) a first container (most preferably sterilised, sterilization methodology well known to those skilled in the art, and packaged/sealed to maintain sterilization; optionally it is a blister pack, wherein this term and its meaning is well known in the art); (b) a pharmaceutical composition located within the first container, wherein the composition, comprises: a first therapeutic agent, comprising: a compound of the present disclosure or a pharmaceutically acceptable salt form thereof; and, (c) a package insert stating that the pharmaceutical composition can be used for the treatment of a specified disease(s)/disorder(s)/condition(s) in a subject, optionally stating the treatment of a disease(s)/disorder(s)/condition(s) refered to herein, optionally stating the treatment of cancer. In another embodiment, the package insert states that the pharmaceutical composition can be used in combination (as defined previously) with a second therapeutic agent(s) to treat the same specified disease(s)/disorder(s) in a subject, optionally wherein this is cancer. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied). Information may be provided by reference to a website(s).

Herein disclosed are articles of manufacture that include a compound(s) and/or pharmaceutical composition(s) described herein in a suitable container. The container may be a vial, jar, ampoule, preloaded syringe, intravenous bag or other used for containing a drug for human or veterinary use. Disclosed herein are kits that include a compound(s) of this disclosure, or a pharmaceutically acceptable salt, solvate, hydrate or prodrug thereof, and suitable packaging, optionally with a package insert, optionally a paper package insert. In some embodiments, a kit further includes instructions for use, which can be upon a package insert. In some embodiments, a kit includes a compound(s) of this disclosure, or a pharmaceutically acceptable salt, solvate, hydrate or prodrug thereof, packaging and a label and/or instructions for use of the compound(s)/composition(s) in the treatment/amelioration/prevention/combating of indications, optionally including one or more diseases/disorders/conditions described herein. Pharmaceutical compound(s)/composition(s) of this disclosure can be inside one or more embodiments of a container/pack/package/dispenser, optionally together with instructions for administration, optionally with drug information such as, for example, disease(s)/disorder(s)/condition(s) treated/ameliorated/prevented/combated, possible side-effects, desirable or undesirable/dangerous drug interactions possible in the subject, information related to what to do upon overdose, and any information specified for inclusion according to the relevant regulatory authority e.g. the FDA in the USA, and optionally wherein each dose (e.g. daily) of compound(s)/composition(s) is compartmentalized in its own compartment or discrete storage within a bigger package, optionally labelled (e.g. with day of week). For example, a packaged product may comprise a container; an effective amount of a compound(s) of this disclosure; and an insert associated with the container, indicating administering the compound(s) for treating a disorder(s), optionally cancer. The composition(s) of the kit may be provided as any suitable form. The kits may contain instructions for mixing, diluting, and/or administrating the compounds. In certain embodiments, the kit provided herein further comprises a device that is used to administer the active ingredients. Examples of such devices include, but are not limited to, syringes, drip bags, patches, and inhalers. The kits also can include other container(s) with pharmaceutically acceptable vehicle(s) that can be used to administer one or more active ingredients, such as one or more solvents, surfactants, preservatives, and/or diluents (e.g., normal saline (0.9% NaCl), or 5% dextrose) as well as containers for mixing, diluting or administering the components to the subject in need of such treatment. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. In a particular embodiment, the solution for administering the compound is sterilised. In some embodiments, the package insert instructs a user of the kit to administer the compound or pharmaceutical composition to a subject. In some embodiments, the package insert instructs a user of the kit to mix the compound or pharmaceutical composition with an aqueous solution. In some embodiments, the package insert instructs a user of the kit to orally administer the compound to the subject. The kits may further comprise conventional pharmaceutical kit components which will be readily apparent to those skilled in the art. Optionally the kit contains a compound(s) of Formula [X] and a further compound or compounds approved for human use by the FDA and/or EMA, for example an anti-cancer drug(s), and optionally instructions for administrating the compounds contained therein to a subject, optionally with materials to perform or assist said administration.

### Polymorphs

In certain embodiments, a compound(s) of Formula [X] is a solid. In certain embodiments, a solid compound(s) of Formula [X] is amorphous (lacks long-range order at the molecular level). In certain embodiments, a solid compound(s) of Formula [X] is crystalline. The term ‘crystalline’ refers to a solid phase in which the material has a regular ordered internal structure at the molecular level and gives a distinctive X-ray diffraction pattern with defined peaks. Compounds of the present disclosure may exist as polymorphs. As used herein “polymorph” refers to crystalline forms having the same chemical composition but different spatial arrangements of the molecules, and/or ions forming the crystal. Co-crystals are typically defined as crystalline complexes of neutral molecular constituents that are bound together through non-covalent interactions, but could also be a complex of a neutral molecule with a salt. Co-crystals may be prepared by melt crystallization, by recrystallization from solvents, or by physically grinding the components together; see O. Almarsson and M. J. Zaworotko, Chem. Commun. 2004, 17, 1889-1896. For a general review of multi-component complexes, see J. K. Haleblian, J. Pharm. Sci. 1975, 64, 1269-1288. Reference to a compound of the Formula [X] herein is understood to include reference to amorphous/crystal/polymorph/co-crystal/clathrate form thereof, unless specified otherwise or otherwise clear from context. A polymorph(s) of a compound(s), or a polymorph of a salt/solvate/hydrate/prodrug of at least one compound of at least one of Formula (I), (II), (III), (IV), (V), (VI), (VII), (VIII), [X], and/or a polymorph(s) of another compound(s) that reduces F₁F₀ ATP hydrolysis, is componentry to this disclosure, as is its incorporation, optionally of a therapeutically effective amount, into a pharmaceutically acceptable composition, optionally with a further polymorph(s) and/or compound(s) of this disclosure, optionally with an FDA and/or EMA approved therapeutic(s). A disclosure embodiment is the use of a polymorph(s) of this disclosure for the manufacture of a medicament for the treatment, amelioration, prevention or combating of a disease or disorder, optionally one or more diseases/disorders/conditions referred to herein. A disclosure embodiment is the use of one or more of these polymorph(s)/composition(s) for use in a method of treatment of the human or animal body by therapy, optionally to treat/ameliorate/prevent/combat one or more diseases/disorders/conditions referred to herein, optionally cancer. In some embodiments, the subject is further administered with one or more compounds or compositions approved for human use, optionally for anti-cancer use, by the United States Food and Drug Administration (FDA) and/or European Medicines Agency (EMA), optionally in the same pharmaceutical composition. An aspect of this disclosure is a pharmaceutical composition comprising at least one polymorph of a compound as described herein and a pharmaceutically-acceptable carrier or diluent.

The crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Bym, R. R. Pfeiffer, and J. G. Stowell, 2^{nd} Edition, SSCI, West Lafayette, Ind., 1999.

### Co-Administration

A compound(s) of Formula [X] can be combined with one or more other pharmacologically active compounds (“second active agents”) in methods and compositions provided herein. As used herein, the term “co-administration” refers to the administration of at least two agent(s)/therapies, one or more of which is a compound or composition of the present disclosure. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s). The use of multiple compounds to treat an indication can increase the beneficial effects while reducing the presence of side effects.

The compounds of the present disclosure may be administered individually, in combination with each other, and/or in combination with other pharmaceuticals/treatments useful for treating the disease or condition of interest. When administered in combination, each component may be administered at the same time or sequentially in any order at different points in time. Thus, each component may be administered separately but sufficiently closely in time so as to provide the desired therapeutic effect, in a way that the therapeutical effects of the first administered one is not entirely dissipated when the subsequent is administered. In one embodiment, the component medications are coformulated for convenience (e.g. in the same injectable or ingestible composition). In an alternative embodiment, the component medications are packaged/distributed/sold/promoted/advertised together.

### Scope

The examples of this disclosure are provided to better illustrate the claimed disclosure and are not to be interpreted in any way as limiting the scope of the disclosure. These specific compounds, compositions, materials, methods and kits are not intended to limit the disclosure, but merely to illustrate specific embodiments falling within the scope of the disclosure. One skilled in the art may develop equivalent compounds, compositions, materials, and methods without the exercise of inventive capacity and without departing from the scope of the disclosure. It is therefore to be understood that within the scope of the appended claims, the disclosure may be practiced otherwise than as specifically described herein. It will be understood that many variations can be made in the procedures herein described while still remaining within the bounds of the disclosure. It is the intention of the inventor that such variations are included within the scope of the disclosure. The present disclosure may be embodied in other specific forms without departing from the spirit or essential attributes thereof. This disclosure encompasses all combinations of preferred aspects of the disclosure noted herein. It is understood that any and all embodiments of the present disclosure may be taken in conjunction with any other embodiment or embodiments to describe additional embodiments. It is also to be understood that each individual element of the embodiments is its own independent embodiment. Furthermore, any element of an embodiment is meant to be combined with any and all other elements from any embodiment to describe an additional embodiment. Feature(s) described in connection with one embodiment of the disclosure may be used in conjunction with another embodiment(s), even if not explicitly stated. Any/all of the features described herein (including any accompanying claims, abstract and drawings), and/or all/some of the steps of any method or process so disclosed, may be combined with any of the above aspects in any combination.

### EQUIVALENTS

Those skilled in the art will recognize, or be able to ascertain using no more than routine experimentation, many equivalents to the specific embodiments of the disclosure described herein. Such equivalents are componentry to this disclosure.

### Abbreviations

All abbreviations used in this disclosure are standard/used in the art, familiar/discernable to a person of the art, especially in their context, to illustrate: Ph=phenyl; Bn=benzyl; Me=methyl; Et=ethyl; MeOH=methanol; EtOH=ethanol; Pr=propyl; Bu=butyl; PE=petroleum ether; COOEt=ethoxycarbonyl; CO2Et=ethoxycarbonyl; Et3SiH=triethylsilane; LiAlH4=lithium aluminium hydride; ACN=acetonitrile=CH₃CN; AcOH=acetic acid; HOAc=acetic acid; Mel=CH₃I; Boc=tert-butyloxycarbonyl protecting group; CDI=1,1′-carbonyldiimidazole; DCE=1,2-Dichloroethane; DCM=dichloromethane; DBU=1,8-diazabicyclo[5,4,0] undec-7-ene; DMAP=4-Dimethylaminopyridine; DMF=N,N-dimethylformamide; DPPA=Diphenylphosphoryl azide; EDCl=N-(3-dimethylaminopropyl)-N′-ethylcarbodiimide hydrochloride or 1-Ethyl-3-(3-dimethylaminopropyl) carbodiimide; EtOAc=ethyl acetate; HEPES=4-(2-hydroxyethyl)-1-piperazineethanesulfonic acid; IBX=2-iodoxybenzoic acid; IPA=isopropanol; MEI=methyl iodide; Tf₂O=triflic anhydride; NaBH (OAc)3=sodium triacetoxyborohydride; NCS═N═C═S; R_{f}=retention value; t-BuOK=potassium tert-butoxide; TEA=triethylamine=Et3N; THF=tetrahydrofuran; TFA=trifluoroacetic acid; TFAA=trifluoroacetic anhydride; T3P=Propanephosphonic acid anhydride; HATU=Hexafluorophosphate Azabenzotriazole Tetramethyl Uronium; HOBt=Hydroxybenzotriazole; TBAF=Tetra-n-butylammonium Fluoride; Cbz=carboxybenzyl group; TBA=Tertiary butyl alcohol; TLC=thin-layer chromatography; SFC=supercritical fluid chromatography or chiral supercritical fluid chromatography; IPAm=isopropylamine; Tol or Tol.=toluene or tolyl; SM=starting material; min=minute(s); h or hr=hour(s); aq. or aq=aqueous.

### SOME PATENTS, OR PATENT APPLICATIONS, CITED

[P1] Atwal K S, Grover G J, Ding C Z, Stein P D, Lloyd J, Ahmad S, Hamann L G, Green D, Ferrara F N, inventors; Bristol-Myers Squibb Co., assignee. (1-phenyl-2-heteoaryl)ethyl-guanidine compounds as inhibitors of mitochondrial F1F0 ATP hydrolase. United States patent U.S. Pat. No. 6,916,813. 2005 Jul. 12. [P2] Ding C, Hamann L, Stein P, Pudzianowski A, inventors; Ding Charles Z., Hamann Lawrence G., Stein Philip D., Pudzianowski Andrew T., assignee. Benzodiazepine inhibitors of mitochondial F1F0 ATP hydrolase and methods of inhibiting F1F0 ATP hydrolase. United States patent application U.S. Ser. No. 10/461,736. 2003 Jun. 13. [P3] Hamann L G, Pudzianowski A T, inventors; Bristol-Myers Squibb Company, assignee. N-substituted phenylurea inhibitors of mitochondrial F1F0 ATP hydrolase. United States patent U.S. Pat. No. 6,846,836. 2005 Jan. 25. [P4] Glick G D, inventor; University of Michigan, assignee. Methods and compositions for treating diseases and conditions associated with mitochondrial function. United States patent application U.S. Ser. No. 11/726,219. 2009 Nov. 5. [P5] Glick G, inventor; University of Michigan, assignee. Methods and compositions for treating diseases and conditions associated with mitochondrial function. United States patent application U.S. Ser. No. 11/110,228. 2005 Dec. 8. [P6] Ding C Z, Atwal K S, inventors; Bristol-Myers Squibb Company, assignee. Sulfonamido substituted benzopyran derivatives. United States patent U.S. Pat. No. 5,869,478. 1999 Feb. 9. [P7] Regnier G, Canevari R, Laubie M, inventors; En Nom Collectif Science Union, Medicale Rech France, assignee. S-triazine compounds. United States patent U.S. Pat. No. 3,647,794. 1972 Mar. 7. [P8] Dax S L, Woodward R, Peng S, inventors; Galleon Pharmaceuticals Inc, assignee. Compounds as respiratory stimulants for treatment of breathing control disorders or diseases. United States patent U.S. Pat. No. 9,351,972. 2016 May 31. [P9] Regnier G, Dhainaut A, Laubie M, Duhault J, inventors; ADIR et Cie, assignee. Disubstituted polymethylene imines. United States patent U.S. Pat. No. 4,514,398. 1985 Apr. 30. [P10] Reginier G, Laubie M, Duhault J, inventors; ADIR et Cie, assignee. Piperazine and homopiperazine compounds. United States patent U.S. Pat. No. 4,492,696. 1985 Jan. 8. [P11] Regnier G, Dhainaut A, Laubie M, Duhault J, inventors; ADIR et Cie, assignee. NN′substituted polymethylene diamines. United States patent U.S. Pat. No. 4,514,399. 1985 Apr. 30. [P12] Regnier G, Dhainaut A, Laubie M, Duhault J, Roman F, inventors; ADIR et Cie, assignee. Anti-hypoxic disubstituted polymethylene imine derivatives, compositions, and method of use therefor. United States patent U.S. Pat. No. 4,593,026. 1986 Jun. 3. [P13] CN105693806A. Chinese patent application. [P14] Christen M Anderson, William Clevenger. Compositions and methods for regulating endogenous inhibitor of ATP synthase, including treatment for diabetes. United States patent application. US 2003/0026781 A1. Published 2003 Feb. 6.

### NON-PATENT REFERENCES

[1] Stryer L, Berg J M, Tymoczko J L (2002) Biochemistry, 4^{th} Ed. New York, NY: WH Freeman. [2]Alberts B, Johnson A, Lewis J, Raff M, Roberts K, Walter P (1994) Molecular Biology Of The Cell, 3^{rd} Ed. New York, NY: Garland Publishing. [3] Nicholls D G, Ferguson S (2013) Bioenergetics. Academic Press. [4] Hong S, Pedersen P L (2008) ATP synthase and the actions of inhibitors utilized to study its roles in human health, disease, and other scientific areas. Microbiology and Molecular Biology Reviews 72(4):590-641. [5] Marvin cheminformatics suite (version 16.3.21; academic license; ChemAxon Kft., Budapest, Hungary; www.chemaxon.com). [6] Gordon C J (2012) Thermal physiology of laboratory mice: Defining thermoneutrality. Journal of Thermal Biology. 37(8):654-85. [7] Ivanes F (2013) New mechanisms of protection of cardiomyocytes from ischemia/reperfusion injury (Doctoral dissertation, Université Claude Bernard-Lyon I). [8] Atwal K S, Ahmad S, Ding C Z, Stein P D, Lloyd J, Hamann L G, Green D W, Ferrara F N, Wang P, Rogers W L, Doweyko L M, Miller A V, Bisaha S N, Schmidt J B, Li L, Yost K J, Lan H J, Madsen C S (2004)N-[1-Aryl-2-(1-imidazolo)ethyl]-guanidine derivatives as potent inhibitors of the bovine mitochondrial F Fo ATP hydrolase. Bioorg. Med. Chem. Lett. 141027-1030. [9] Atwal K S, Wang P, Rogers W L, Sleph P, Monshizadegan H, Ferrara F N, Traeger S, Green D W, Grover G J (2004) Small molecule mitochondrial F Fo ATPase hydrolase inhibitors as cardioprotective agents. Identification of 4-(N-arylimidazole)-substituted benzopyran derivatives as selective hydrolase inhibitors. J. Med. Chem. 471081-1084. INCLUDING this paper's supplementary material. [10] Hamann L G, Ding C Z, Miller A V, Madsen C S, Wang P, Stein P D, Pudzianowski A T, Green D W, Monshizadegan H, Atwal K S (2004) Benzodiazepine-based selective inhibitors of mitochondrial F₁F₀ ATP hydrolase. Bioorg. Med. Chem. Lett. 141031-1034. [11] Grover G J, Atwal K S, Sleph P G, Wang F L, Monshizadegan H, Monticello T, Green D W (2004) Excessive ATP hydrolysis in ischemic myocardium by mitochondrial F1F0-ATPase: effect of selective pharmacological inhibition of mitochondrial ATPase hydrolase activity. American Journal of Physiology-Heart and Circulatory Physiology 287(4):H1747-H1755. [12] Ivanes F, Faccenda D, Gatliff J, Ahmed A A, Cocco S, Cheng CHK, . . . & Campanella M (2014) The compound BTB06584 is an IF1-dependent selective inhibitor of the mitochondrial F1Fo-ATPase. British journal of pharmacology 171(18):4193-4206. [13] Zheng J, Ramirez V D (2000) Inhibition of mitochondrial proton FOF1-ATPase/ATP synthase by polyphenolic phytochemicals. British journal of pharmacology. 130(5):1115-23. [14] Yin X, Giap C, Lazo J S, et al. (2003) Low molecular weight inhibitors of Myc-Max interaction and function. Oncogene. 22(40):6151-9. [15] Ng J, Shuryak I (2015) Minimizing second cancer risk following radiotherapy: current perspectives. Cancer management and research. 7:1. [16] Armstrong G T, Kawashima T, Leisenring W, Stratton K, Stovall M, Hudson M M, Sklar C A, Robison L L, Oeffmger K C (2014) Aging and risk of severe, disabling, life-threatening, and fatal events in the childhood cancer survivor study. Journal of clinical oncology. 32(12):1218. [17] Castro R F, Azzalis L A, Feder D, Perazzo F F, Pereira E C, Junqueira V B, Rocha K C, Machado C D, Paschoal F C, Gnann L A, Fonseca F L (2012) Safety and efficacy analysis of liposomal insulin-like growth factor-1 in a fluid gel formulation for hair-loss treatment in a hamster model. Clinical and Experimental Dermatology: Experimental dermatology. 37(8):909-12. [18] Patel A, Cholkar K, Agrahari V, Mitra A K (2013) Ocular drug delivery systems: an overview. World journal of pharmacology. 2(2):47. [19] Gaudana R, Ananthula H K, Parenky A, Mitra A K (2010) Ocular drug delivery. The AAPS journal. 12(3):348-60. [20] Mitra A K, Anand B S, Duvvuri S (2005) Drug delivery to the eye. Advances in Organ Biology. 10:307-51. [21] Wong W L, Su X, Li X, Cheung C M, Klein R, Cheng C Y, Wong T Y (2014) Global prevalence of age-related macular degeneration and disease burden projection for 2020 and 2040: a systematic review and meta-analysis. The Lancet Global Health. 2(2):e106-16. [22] Hao J, Li S K (2019) Inner ear drug delivery: Recent advances, challenges, and perspective. European Journal of Pharmaceutical Sciences. 126:82-92. [23] Liu H, Hao J, Li K S (2013) Current strategies for drug delivery to the inner ear. Acta Pharmaceutica Sinica B. 3(2):86-96. 1241 Lin M T, Beal M F (2006) Mitochondrial dysfunction and oxidative stress in neurodegenerative diseases. Nature. 443(7113):787-95. [25] Umphred D A, Lazaro R T, Roller M, Burton G, editors Neurological rehabilitation. Elsevier Health Sciences; 2013 Aug 7. [26] Kang Y S, Jung H J, Oh J S, Song D Y (2016) Use of PEGylated Immunoliposomes to Deliver Dopamine Across the Blood-Brain Barrier in a Rat Model of Parkinson's Disease. CNS Neuroscience & Therapeutics. 22(10):817-23. [27] Di Gioia S, Trapani A, Mandracchia D, De Giglio E, Cometa S, Mangini V, Arnesano F, Belgiovine G, Castellani S, Pace L, Lavecchia M A (2015) Intranasal delivery of dopamine to the striatum using glycol chitosan/sulfobutylether-β-cyclodextrin based nanoparticles. European Journal of Pharmaceutics and Biopharmaceutics. 94:180-93. [28] Oorschot D E. Total number of neurons in the neostriatal, pallidal, subthalamic, and substantia nigral nuclei of the rat basal ganglia: a stereological study using the cavalieri and optical disector methods. J Comp Neurol. 1996; 366:580-599. [29] Naoi M, Maruyama W (1999) Cell death of dopamine neurons in aging and Parkinson's disease. Mechanisms of ageing and development. 111(2):175-88. [30] Marschner J A, Schäfer H, Holderied A, Anders H J (2016) Optimizing mouse surgery with online rectal temperature monitoring and preoperative heat supply. Effects on post-ischemic acute kidney injury. PLoS One. 1 1(2):e0149489. [31] Strong R, Miller R A, Astle C M, Baur J A, De Cabo R, Fernandez E, Guo W, Javors M, Kirkland J L, Nelson J F, Sinclair D A (2012) Evaluation of resveratrol, green tea extract, curcumin, oxaloacetic acid, and medium-chain triglyceride oil on life span of genetically heterogeneous mice. Journals of Gerontology Series A: Biomedical Sciences and Medical Sciences. 68(1):6-16. [32] Harrison D E, Strong R, Sharp Z D, Nelson J F, Astle C M, Flurkey K, Nadon N L, Wilkinson J E, Frenkel K, Carter C S, Pahor M (2009) Rapamycin fed late in life extends lifespan in genetically heterogeneous mice. Nature. 460(7253):392. [33] Colman R J, Anderson R M, Johnson S C, Kastman E K, Kosmatka K J, Beasley T M, Allison D B, Cruzen C, Simmons H A, Kemnitz J W, Weindruch R (2009) Caloric restriction delays disease onset and mortality in rhesus monkeys. Science. 325(5937):201-4. [34] Ackert-Bicknell C L, Anderson L C, Sheehan S, Hill W G, Chang B, Churchill G A, Chesler E J, Korstanje R, Peters L L (2017) Aging research using mouse models. Current protocols in mouse biology. 5(2):95-133. [35] Sukoff Rizzo S J, Anderson L C, Green T L, McGarr T, Wells G, Winter S S (2018) Assessing Healthspan and Lifespan Measures in Aging Mice: Optimization of Testing Protocols, Replicability, and Rater Reliability. Current Protocols in Mouse Biology. 8(2):e45. [36] Richardson A, Fischer K E, Speakman J R, De Cabo R, Mitchell S J, Peterson C A, Rabinovitch P, Chiao Y A, Taffet G, Miller R A, Renterfa R C (2015) Measures of healthspan as indices of aging in mice—a recommendation. Journals of Gerontology Series A: Biomedical Sciences and Medical Sciences. 71(4):427-30. Refer especially to this paper's Supplementary Material. [37] Vermeij W P, Hoeij makers J H, Pothof J (2016) Genome integrity in aging: human syndromes, mouse models, and therapeutic options. Annual review of pharmacology and toxicology. 56:427-45. [38] Harkema L, Youssef S A, de Bruin A (2016) Pathology of mouse models of accelerated aging. Veterinary pathology. 53(2):366-89. [39] Hasty P, Campisi J, Hoeijmakers J, Van Steeg H, Vijg J (2003) Aging and genome maintenance: lessons from the mouse?Science. 299(5611):1355-9. [40] de Boer J, Hoeijmakers J H (1999) Cancer from the outside, aging from the inside: mouse models to study the consequences of defective nucleotide excision repair. Biochimie. 81(1-2):127-37. [41] Baker D J, Jeganathan K B, Cameron J D, Thompson M, Juneja S, Kopecka A, Kumar R, Jenkins R B, De Groen P C, Roche P, Van Deursen J M (2004) BubR1 insufficiency causes early onset of aging-associated phenotypes and infertility in mice. Nature genetics. 36(7):744. [42] de Boer J, de Wit J, van Steeg H, Berg R J, Morreau H, Visser P, Lehmann A R, Duran M, Hoeijmakers J H, Weeda G (1998) A mouse model for the basal transcription/DNA repair syndrome trichothiodystrophy. Molecular cell. 1(7):981-90. [43] De Boer J, Andressoo J O, de Wit J, Huijmans J, Beems R B, van Steeg H, Weeda G, van der Horst G T, van Leeuwen W, Themmen A P, Meradji M (2002) Premature aging in mice deficient in DNA repair and transcription. Science. 296(5571):1276-9. [44] Andressoo J O, Mitchell J R, de Wit J, Hoogstraten D, Volker M, Toussaint W, Speksnijder E, Beems R B, van Steeg H, Jans J, de Zeeuw C I (2006) An Xpd mouse model for the combined xeroderma pigmentosum/Cockayne syndrome exhibiting both cancer predisposition and segmental progeria. Cancer cell. 10(2):121-32. [45] Cheo D L, Ruven H J, Meira L B, Hammer R E, Burns D K, Tappe N J, van Zeeland A A, Mullenders L H, Friedberg E C (1997) Characterization of defective nucleotide excision repair in XPC mutant mice. Mutation Research/Fundamental and Molecular Mechanisms of Mutagenesis. 374(1):1-9. [46] Melis J P, Wijnhoven S W, Beems R B, Roodbergen M, Van Den Berg J, Moon H, Friedberg E, Van Der Horst G T, Hoeijmakers J H, Vijg J, Van Steeg H (2008) Mouse models for xeroderma pigmentosum group A and group C show divergent cancer phenotypes. Cancer research. 68(5):1347-53. [47] Niedernhofer U, Garinis G A, Raams A, Lalai A S, Robinson A R, Appeldoorn E, Odijk H, Oostendorp R, Ahmad A, Van Leeuwen W, Theil A F (2006) A new progeroid syndrome reveals that genotoxic stress suppresses the somatotroph axis. Nature. 444(7122):1038. [48] Weeda G, Donker I, de Wit J, Morreau H, Janssens R, Vissers C, Nigg A, van Steeg H, Bootsma D, Hoeijmakers J H (1997) Disruption of mouse ERCC1 results in a novel repair syndrome with growth failure, nuclear abnormalities and senescence. Current Biology. 7(6):427-39. [49] Spoor M, Nagtegaal A P, Ridwan Y, Borgesius N Z, van Alphen B, van der Pluijm I, Hoeijmakers J H, Frens M A, Borst J G (2012) Accelerated loss of hearing and vision in the DNA-repair deficient Ercc1δ/− mouse. Mechanisms of ageing and development. 133(2-3):59-67. [50] Vogel H, Lim D S, Karsenty G, Finegold M, Hasty P (1999) Deletion of Ku86 causes early onset of senescence in mice. Proceedings of the National Academy of Sciences. 96(19):10770-5. [51] Reiling E, Dolld M E, Youssef S A, Lee M, Nagarajah B, Roodbergen M, De Bruin A, Hoeijmakers J H, Vijg J, Van Steeg H, Hasty P (2014) The progeroid phenotype of Ku80 deficiency is dominant over DNA-PKCS deficiency. PloS one. 9(4):e93568. [52] Zhang Y, Padalecki S S, Chaudhuri A R, De Waal E, Goins B A, Grubbs B, Ikeno Y, Richardson A, Mundy G R, Herman B (2007) Caspase-2 deficiency enhances aging-related traits in mice. Mechanisms of ageing and development. 128(2):213-21. [53] Bergeron L, Perez G I, Macdonald G, Shi L, Sun Y, Jurisicova A, Varmuza S, Latham K E, Flaws J A, Salter J C, Hara H (1998) Defects in regulation of apoptosis in caspase-2-deficient mice. Genes & development. 12(9):1304-14. [54] Nadon N L, Strong R, Miller R A, Nelson J, Javors M, Sharp Z D, Peralba J M, Harrison D E (2008) Design of aging intervention studies: the NIA interventions testing program. Age. 30(4):187-99. [55] Miller R A, Harrison D E, Astle C M, Floyd R A, Flurkey K, Hensley K L, Javors M A, Leeuwenburgh C, Nelson J F, Ongini E, Nadon N L (2007) An aging interventions testing program: study design and interim report. Aging cell. 6(4):565-75. [56] Martin-Montalvo A, Mercken E M, Mitchell S J, Palacios H H, Mote P L, Scheibye-Knudsen M, Gomes A P, Ward T M, Minor R K, Blouin M J, Schwab M (2013) Metformin improves healthspan and lifespan in mice. Nature communications. 4:2192. [57] Miller R A, Harrison D E, Astle C M, Baur J A, Boyd A R, De Cabo R, Fernandez E, Flurkey K, Javors M A, Nelson J F, Orihuela C J (2011) Rapamycin, but not resveratrol or simvastatin, extends life span of genetically heterogeneous mice. The Journals of Gerontology: Series A. 66(2):191-201. [58] Strong R, Miller R A, Astle C M, Floyd R A, Flurkey K, Hensley K L, Javors M A, Leeuwenburgh C, Nelson J F, Ongini E, Nadon N L (2008) Nordihydroguaiaretic acid and aspirin increase lifespan of genetically heterogeneous male mice. Aging cell. 7(5):641-50. [59] Baur J A, Pearson K J, Price N L, Jamieson H A, Lerin C, Kalra A, Prabhu V V, Allard J S, Lopez-Lluch G, Lewis K, Pistell P J (2006) Resveratrol improves health and survival of mice on a high-calorie diet. Nature. 444(7117):337. [60] Spilman P, Podlutskaya N, Hart M J, Debnath J, Gorostiza O, Bredesen D, Richardson A, Strong R, Galvan V (2010) Inhibition of mTOR by rapamycin abolishes cognitive deficits and reduces amyloid-p levels in a mouse model of Alzheimer's disease. PloS one. 5(4):e9979. [61] Morley J E, Armbrecht H J, Farr S A, Kumar V B (2012) The senescence accelerated mouse (SAMP8) as a model for oxidative stress and Alzheimer's disease. Biochimica et Biophysica Acta (BBA)-Molecular Basis of Disease. 1822(5):650-6. [62] Peto M V, De la Guardia C, Winslow K, Ho A, Fortney K, Morgen E (2017) MortalityPredictors.org: a manually-curated database of published biomarkers of human all-cause mortality. Aging (Albany NY). 9(8):1916. [63] Cheng D, Logge W, Low J K, Garner B, Karl T (2013) Novel behavioural characteristics of the APP Swe/PS1ΔE9 transgenic mouse model of Alzheimer's disease. Behavioural brain research. 245:120-7. [64] Richard G.M. Morris (2008) Morris water maze. Scholarpedia, 3(8):6315. [65] Spoor M, Nagtegaal A P, Ridwan Y, Borgesius N Z, van Alphen B, van der Pluijm I, Hoeijmakers J H, Frens M A, Borst J G (2012) Accelerated loss of hearing and vision in the DNA-repair deficient Ercc1δ/− mouse. Mechanisms of ageing and development. 133(2-3):59-67. [66]Borgesius N Z, de Waard M C, van der Pluijm I, Omrani A, Zondag G C, van der Horst G T, Melton D W, Hoeijmakers J H, Jaarsma D, Elgersma Y (2011) Accelerated age-related cognitive decline and neurodegeneration, caused by deficient DNA repair. Journal of Neuroscience. 31(35):12543-53. [67] Yurko-Mauro K (2010) Cognitive and cardiovascular benefits of docosahexaenoic acid in aging and cognitive decline. Current Alzheimer Research. 7(3):190-6. [68] Zahr N M, Mayer D, Pfefferbaum A, Sullivan E V (2008) Low striatal glutamate levels underlie cognitive decline in the elderly: evidence from in vivo molecular spectroscopy. Cerebral Cortex. 18(10):2241-50. [69] Rupsingh R, Borrie M, Smith M, Wells J L, Bartha R (2011) Reduced hippocampal glutamate in Alzheimer disease. Neurobiology of aging. 32(5):802-10. [70] Fayed N, Modrego P J, Rojas-Salinas G, Aguilar K (2011) Brain glutamate levels are decreased in Alzheimer's disease: a magnetic resonance spectroscopy study. American Journal of Alzheimer's Disease & Other Dementias. (6):450-6. [71] Matjusaitis M, Chin G, Sarnoski E A, Stolzing A (2016) Biomarkers to identify and isolate senescent cells. Ageing research reviews. 29:1-2. [72] Gorisse L, Pietrement C, Vuiblet V, Schmelzer C E, Köhler M, Duca L, Debelle L, Fornès P, Jaisson S, Gillery P (2016) Protein carbamylation is a hallmark of aging. Proceedings of the National Academy of Sciences. 113(5):1191-6. [73] Syslová K, Böhmova A, Mikoška M, Kuzma M, Pelclová D, Kačer P (2014) Multimarker screening of oxidative stress in aging. Oxidative medicine and cellular longevity. 2014. [74] Stadtman E R, Berlett B S (1998) Reactive oxygen-mediated protein oxidation in aging and disease. Drug metabolism reviews. 30(2):225-43. [75] Nomura Y, Wang B X, Qi S B, Namba T, Kaneko S (1989) Biochemical changes related to aging in the senescence-accelerated mouse. Experimental gerontology. 24(1):49-55. [76] Kitamura Y, Zhao X H, Ohnuki T, Takei M, Nomura Y (1992) Age-related changes in transmitter glutamate and NMDA receptor/channels in the brain of senescence-accelerated mouse. Neuroscience letters. 137(2):169-72. [77] Nomura Y, Kitamura Y, Ohnuki T, Arima T, Yamanaka Y, Sasaki K, Oomura Y (1997) Alterations in acetylcholine, NMDA, benzodiazepine receptors and protein kinase C in the brain of the senescence-accelerated mouse: an animal model useful for studies on cognitive enhancers. Behavioural brain research. 83(1-2):51-5. [78] Oomura Y, Sasaki K, Li A, Yoshh H, Fukata Y, Yago H, Kimura H, Tooyama I, Hanai K, Nomura Y, Yanaihara N (1996) Protection against Impairment of Memory and Immunoreactivity in Senescence-Accelerated Mice by Acidic Fibroblast Growth Factora. Annals of the New York Academy of Sciences. 786(1):337-47. [79] Nomura Y, Okuma Y (1999) Age-related defects in lifespan and learning ability in SAMP8 mice. Neurobiology of aging. 20(2):111-5. [80] Tomobe K, Nomura Y (2009) Neurochemistry, neuropathology, and heredity in SAMP8: a mouse model of senescence. Neurochemical research. 34(4):660-9. [81] Zhao X H, Kitamura Y, Nomura Y (1992) Age-related changes in NMDA-induced [3H] acetylcholine release from brain slices of senescene-accelerated mouse. International journal of developmental neuroscience. 10(2):121-9. [82] Benjamin S A, Finch C E, Guerin J C, Nelson J F, Olshansky S J, Roth G, Smith R G (2011) Handbook of models for human aging. Elsevier. [83] Mills K F, Yoshida S, Stein L R, Grozio A, Kubota S, Sasaki Y, Redpath P, Migaud M E, Apte R S, Uchida K, Yoshino J (2016) Long-term administration of nicotinamide mononucleotide mitigates age-associated physiological decline in mice. Cell metabolism. 24(6):795-806. [84] Rangaraju S, Solis G M, Thompson R C, Gomez-Amaro R L, Kurian L, Encalada S E, Niculescu III A B, Salomon D R, Petrascheck M (2015) Suppression of transcriptional drift extends C. elegans lifespan by postponing the onset of mortality. Elife. 4:e08833. [85] Barger J L, Anderson R M, Newton M A, da Silva C, Vann J A, Pugh T D, Someya S, Prolla T A, Weindruch R (2015) A conserved transcriptional signature of delayed aging and reduced disease vulnerability is partially mediated by SIRT3. PLoS One. 10(4):e0120738. [86] Yaku K, Okabe K, Nakagawa T (2018) NAD metabolism: Implications in aging and longevity. Ageing research reviews. [87] Ivanisevic J, Stauch K L, Petrascheck M, Benton H P, Epstein A A, Fang M, Gorantla S, Tran M, Hoang L, Kurczy M E, Boska M D (2016) Metabolic drift in the aging brain. Aging (Albany NY). 8(5):1000. [88] Huxley A F (1959) Ion movements during nerve activity. Annals of the New York Academy of Sciences. 81(2):221-46. [89] Chapman R A (1967) Dependence on temperature of the conduction velocity of the action potential of the squid giant axon. Nature. 213(5081):1143. [90] Maldonado C A, Wooley B D, Pancrazio J J. The excitatory effect of temperature on the Hodgkin-Huxley model. [91] Fitzhugh R (1966) Theoretical effect of temperature on threshold in the Hodgkin-Huxley nerve model. The Journal of general physiology. 49(5):989-1005. [92] Kuang S, Wang J, Zeng T, Cao A (2008) Thermal impact on spiking properties in Hodgkin-Huxley neuron with synaptic stimulus. Pramana. 70(1):183-90. [93] Goldin M A, Mindlin G B (2017) Temperature manipulation of neuronal dynamics in a forebrain motor control nucleus. PLoS computational biology. 13(8):e1005699. [94] Garedew A, Henderson S O, Moncada S (2010) Activated macrophages utilize glycolytic ATP to maintain mitochondrial membrane potential and prevent apoptotic cell death. Cell Death & Differentiation. 17(10):1540-50. [95] Appelberg K S, Wallet M A, Taylor J P, Cash M N, Sleasman J W, Goodenow M M. HIV-1 Infection Primes Macrophages through STAT Signaling to Promote Enhanced Inflammation and Viral Replication (2017) AIDS Research and Human Retroviruses. [96] Burdo T H, Lentz M R, Autissier P, Krishnan A, Halpern E, Letendre S, Rosenberg E S, Ellis R J, Williams K C (2011) Soluble CD163 made by monocyte/macrophages is a novel marker of HIV activity in early and chronic infection prior to and after anti-retroviral therapy. Journal of Infectious Diseases. 204(1):154-63. [97] Blond D, Raoul H, Le Grand R, Dormont D (2000) Nitric oxide synthesis enhances human immunodeficiency virus replication in primary human macrophages. Journal of virology. 74(19):8904-12. [98] Blond D, Cheret A, Raoul H, Le Grand R, Caufour P, Theodoro F, Dormont D (1998) Nitric oxide synthesis during acute SIVmac251 infection of macaques. Research in virology. 149(2):75-86. [99] Rostasy K, Monti L, Yiannoutsos C, Kneissl M, Bell J, Kemper T L, Hedreen J C, Navia B A (1999) Human immunodeficiency virus infection, inducible nitric oxide synthase expression, and microglial activation: pathogenetic relationship to the acquired immunodeficiency syndrome dementia complex. Annals of neurology. 46(2):207-16. [100] Torre D, Ferrario G (1996) Immunological aspects of nitric oxide in HIV-1 infection. Medical hypotheses. 47(5):405-7. [101] Arafnga M, Edagwa B, Mosley R L, Poluektova L Y, Gorantla S, Gendelman H E. A mature macrophage is a principal HIV-1 cellular reservoir in humanized mice after treatment with long acting antiretroviral therapy (2017) Retrovirology. 14(1):17. [102] Lamers S L, Salemi M, Galligan D C, De Oliveira T, Fogel G B, Granier S C, Zhao L, Brown J N, Morris A, Masliah E, McGrath M S (2009) Extensive HIV-1 intra-host recombination is common in tissues with abnormal histopathology. PloS one. 4(3):e5065. [103] Karimi M, Sahandi Zangabad P, Ghasemi A, Amiri M, Bahrami M, Malekzad H, Ghahramanzadeh Asl H, Mahdieh Z, Bozorgomid M, Ghasemi A, Rahmani Taji Boyuk M R (2016) Temperature-responsive smart nanocarriers for delivery of therapeutic agents: applications and recent advances. ACS applied materials & interfaces. 8(33):21107-33. [104] Ta T, Porter T M (2013) Thermosensitive liposomes for localized delivery and triggered release of chemotherapy. Journal of controlled release. 169(1-2):112-25. [105] Zangabad P S, Mirkiani S, Shahsavari S, Masoudi B, Masroor M, Hamed H, Jafari Z, Taghipour Y D, Hashemi H, Karimi M, Hamblin M R (2017) Stimulus-responsive liposomes as smart nanoplatforms for drug delivery applications. Nanotechnology reviews. [106] Kneidl B, Peller M, Winter G, Lindner L H, Hossann M (2014) Thermosensitive liposomal drug delivery systems: state of the art review. International journal of nanomedicine. 9:4387. [107] Shoemaker R H (2006) The NCI60 human tumour cell line anticancer drug screen. Nature Rev Cancer 6:813-23. [108] NCI-60 Screening Methodology. Details of both the one-dose and five-dose assays. https://dtp.cancer.gov/discovery_development/nci-60/methodology.htm (accessed on 25th June, 2017). [109] Bisaha S N, Malley M F, Pudzianowski A, Monshizadegan H, Wang P, Madsen C S, Gougoutas J Z, Stein P D (2005) A switch in enantiomer preference between mitochondrial F1F0-ATPase chemotypes. Bioorganic & medicinal chemistry letters 15(11):2749-51.
[110] Lykkeberg A K, Cornett C, Halling-Sørensen B, Hansen S H (2006) Isolation and structural elucidation of tiamulin metabolites formed in liver microsomes of pigs. Journal of pharmaceutical and biomedical analysis. 42(2):223-231. [111] Gillotin F, Chiap P, Frédérich M, Van Heugen J C, Francotte P, Lebrun P, . . . De Tullio P (2010) Coupling of liquid chromatography/tandem mass spectrometry and liquid chromatography/solid-phase extraction/NMR techniques for the structural identification of metabolites following in vitro biotransformation of SUR1-selective ATP-sensitive potassium channel openers. Drug metabolism and disposition. 38(2):232-240. [112] Holbeck S L, Collins J M, Doroshow J H (2010) Analysis of Food and Drug Administration-approved anticancer agents in the NCI60 panel of human tumor cell lines. Molecular cancer therapeutics. 9(5):1451-60. [113] National Cancer Institute (NCI) Developmental Therapeutics Program (DTP) Screening Data Database https://dtp.cancer.gov/dtpstandard/dwindex/index.jsp. Accessed on 29/06/17. [114] Rouslin W I (1987) The mitochondrial adenosine 5′-triphosphatase in slow and fast heart rate hearts. American Journal of Physiology-Heart and Circulatory Physiology. 252(3):H622-7. [115] Tacutu R, Thornton D, Johnson E, Budovsky A, Barardo D, Craig T, Diana E, Lehmann G, Toren D, Wang J, Fraifeld V E, de Magalhaes J P (2018) Human Ageing Genomic Resources: new and updated databases. Nucleic Acids Research 46(D1):D1083-D1090. [116] Wilson D J (2019) The harmonic mean p-value for combining dependent tests. Proceedings of the National Academy of Sciences. 116(4):1195-1200. [117] Rouslin W, Broge C W (1994) Analysis of factors affecting functional assays for estimating IF1, the mitochondrial ATPase Inhibitor. Analytical biochemistry. 222(1):68-75. [118] Rouslin W (1988) Factors affecting the loss of mitochondrial function during zero-flow ischemia (autolysis) in slow and fast heart-rate hearts. Journal of molecular and cellular cardiology. 20(11):999-1007. [119] Rouslin W, Broge C W (1989) Regulation of mitochondrial matrix pH and adenosine 5′-triphosphatase activity during ischemia in slow heart-rate hearts. Role of Pi/H+ symport. Journal of Biological Chemistry. 264(26):15224-9. [120] Rouslin W, Broge C W (1990) Regulation of the mitochondrial adenosine 5′-triphosphatase in situ during ischemia and in vitro in intact and sonicated mitochondria from slow and fast heart-rate hearts. Archives of biochemistry and biophysics. 280(1):103-11. [121] Rouslin W I, Broge C W, Grupp I L (1990) ATP depletion and mitochondrial functional loss during ischemia in slow and fast heart-rate hearts. American Journal of Physiology-Heart and Circulatory Physiology. 259(6):H1759-66. [122] Rouslin W, Broge C W (1992) Why the Mitochondrial ATPase Inhibitor IF1 Fails to Inhibit the Mitochondrial ATPase in Situ in Fast Heart-Rate Mammalian and Avian Heartsa. Annals of the New York Academy of Sciences. 671(1):505-6. [123] Rouslin W, Broge C W (1993) Factors affecting the species-homologous and species-heterologous binding of mitochondrial ATPase inhibitor, IF1, to the mitochondrial ATPase of slow and fast heart-rate hearts. Archives of biochemistry and biophysics. 303(2):443-50. [124] Rouslin W I, Broge C W (1993) Mechanisms of ATP conservation during ischemia in slow and fast heart rate hearts. American Journal of Physiology-Cell Physiology. 264(1):C209-16. [125] Rouslin W, Frank G D, Broge C W (1995) Content and binding characteristics of the mitochondrial ATPase inhibitor, IF1 in the tissues of several slow and fast heart-rate homeothermic species and in two poikilotherms. Journal of bioenergetics and biomembranes. 27(1):117-25. [126] Rouslin W, Broge C W, Guerrieri F, Capozza G (1995) ATPase activity, IF1 content, and proton conductivity of ESMP from control and ischemic slow and fast heart-rate hearts. Journal of bioenergetics and biomembranes. 27(4):459-66. [127] Rouslin W, Broge C W (1996) IF1 function in situ in uncoupler-challenged ischemic rabbit, rat, and pigeon hearts. Journal of Biological Chemistry. 271(39):23638-41. [128] How does metabolic rate scale with size?Cell biology by the numbers. http://book.bionumbers.org/how-does-metabolic-rate-scale-with-size/. [129] Bertholet A M, Chouchani E T, Kazak L, Angelin A, Fedorenko A, Long J Z, . . . Kirichok Y (2019) H+ transport is an integral function of the mitochondrial ADP/ATP carrier. Nature. 571(7766):515-520. [130] Formentini L, Pereira M P, Sánchez-Cenizo L, Santacatterina F, Lucas J J, Navarro C, Martinez-Serrano A, Cuezva J M (2014) In vivo inhibition of the mitochondrial H+-ATP synthase in neurons promotes metabolic preconditioning. The EMBO journal. 33(7):762-78. [131] Mayford M, Bach M E, Huang Y Y, Wang L, Hawkins R D, Kandel E R (1996) Control of memory formation through regulated expression of a CaMKII transgene. Science. 274(5293):1678-83. [132] De Grey A D (1999) The mitochondrial free radical theory of aging. Austin, T X: R G Landes. [133] Sohal R S, Orr W C (2012) The redox stress hypothesis of aging. Free Radical Biology and Medicine. 52(3):539-55. [134] Wang P, Powell S R (2010) Decreased sensitivity associated with an altered formulation of a commercially available kit for detection of protein carbonyls. Free Radical Biology and Medicine. 49(2):119-21. [135] Santacatterina F, Sánchez-Cenizo L, Formentini L, Mobasher M A, Casas E, Rueda C B, Martfnez-Reyes I, de Arenas C N, García-Bermúdez J, Zapata J M, Sánchez-Aragó M (2016) Down-regulation of oxidative phosphorylation in the liver by expression of the ATPase inhibitory factor 1 induces a tumor-promoter metabolic state. Oncotarget. 7(1):490. [136] Formentini L, Santacatterina F, de Arenas C N, Stamatakis K, López-Martinez D, Logan A, Fresno M, Smits R, Murphy M P, Cuezva J M (2017) Mitochondrial ROS production protects the intestine from inflammation through functional M2 macrophage polarization. Cell reports. 19(6):1202-13. [137] Garcia-Bermúdez J, Sánchez-Aragó M, Soldevilla B, del Arco A, Nuevo-Tapioles C, Cuezva J M (2015) PKA phosphorylates the ATPase inhibitory factor 1 and inactivates its capacity to bind and inhibit the mitochondrial H+-ATP synthase. Cell reports. 12(12):2143-55. [138] Schnizer R, Van Heeke G, Amaturo D, Schuster S M (1996) Histidine-49 is necessary for the pH-dependent transition between active and inactive states of the bovine F1-ATPase inhibitor protein. Biochimica et Biophysica Acta (BBA)-Protein Structure and Molecular Enzymology. 1292(2):241-8. [139] Ando C, Ichikawa N (2008) Glutamic acid in the inhibitory site of mitochondrial ATPase inhibitor, IF1, participates in pH sensing in both mammals and yeast. Journal of biochemistry. 144(4):547-53. [140] Ichikawa N, Ushida S, Kawabata M, Masazumi Y (1999) Nucleotide sequence of cDNA coding the mitochondrial precursor protein of the ATPase inhibitor from humans. Bioscience, biotechnology, and biochemistry. 63(12):2225-7. [141] Green D W, Grover G J (2000) The IF1 inhibitor protein of the mitochondrial F1F0-ATPase. Biochimica et Biophysica Acta (BBA)-Bioenergetics. 1458(2-3):343-55. [142] van Raaij M J, Orriss G L, Montgomery M G, Runswick M J, Fearnley I M, Skehel J M, Walker J E (1996) The ATPase inhibitor protein from bovine heart mitochondria: the minimal inhibitory sequence. Biochemistry. 35(49):15618-25. [143] Gledhill J R, Montgomery M G, Leslie A G, Walker J E (2007) How the regulatory protein, IF1, inhibits F1-ATPase from bovine mitochondria. Proceedings of the National Academy of Sciences. 104(40):15671-6. [144] Papa S, Zanotti F, Cocco T, Perrucci C, Candita C, Minuto M (1996) Identification of functional domains and critical residues in the adenosinetriphosphatase inhibitor protein of mitochondrial F0F1 ATP synthase. European journal of biochemistry. 240(2):461-7. [145] Zanotti F, Raho G, Vuolo R, Gaballo A, Papa F, Papa S (2000) Functional domains of the ATPase inhibitor protein from bovine heart mitochondria. FEBS letters. 482(1-2):163-6. [146] Zanotti F, Raho G, Gaballo A, Papa S (2004) Inhibitory and anchoring domains in the ATPase inhibitor protein IF1 of bovine heart mitochondrial ATP synthase. Journal of bioenergetics and biomembranes. 36(5):447-57. [147] de Chiara C, Nicastro G, Spisni A, Zanotti F, Cocco T, Papa S (2002) Activity and NMR structure of synthetic peptides of the bovine ATPase inhibitor protein, IF1. Peptides. 23(12):2127-41. [148] Stout J S, Partridge B E, Dibbern D A, Schuster S M (1993) Peptide analogs of the beef heart mitochondrial F1-ATPase inhibitor protein. Biochemistry. 32(29):7496-502. [149] Prugh J D, Birchenough L A, Egbertson M S (1992) A simple method of protecting a secondary amine with tert butyloxycarbonyl (BOC) in the presence of a primary amine. Synthetic communications. 22(16):2357-60. [150] Laduron F, Tamborowski V, Moens L, Horvith A, De Smaele D, Leurs S (2005) Efficient and scalable method for the selective alkylation and acylation of secondary amines in the presence of primary amines. Organic process research & development. 9(1):102-4. [151] Gordon B H (1995) The pharmacokinetics and metabolism of almitrine bismesylate. Doctoral dissertation, College of Medicine, Biological Sciences and Psychology, University of Leicester. [152] Travis, William D; Brambilla, Elizabeth; Muller-Hermelink, H Konrad; et al., eds. (2004). Pathology and Genetics of Tumours of the Lung, Pleura, Thymus and Heart. World Health Organization Classification of Tumours. Lyon: IARC Press. ISBN 92 832 2418 3. [153] Cotten J F (2014) The latest pharmacologic ventilator. Anesthesiology: The Journal of the American Society of Anesthesiologists. 121(3):442-4. [154] Golder F J, Hewitt M M, McLeod J F (2013) Respiratory stimulant drugs in the post-operative setting. Respiratory physiology & neurobiology. 189(2):395-402. [155] Yang X, Yu Y, Xu J, Shu H, Liu H, Wu Y, . . . & Wang Y (2020) Clinical course and outcomes of critically ill patients with SARS-CoV-2 pneumonia in Wuhan, China: a single-centered, retrospective, observational study. The Lancet Respiratory Medicine. [156] South East London Joint Medicines Formulary, NHS, http://www.selondonjointmedicinesformulary.nhs.uk/chaptersSubDetails.asp?FormularySectionID=3&Su bSectionRef=03.05.01&SubSectionID=A100&drugmatch=4923 #4923 (accessed on 22/07/18). [157] https://www.has-sante.fr/portail/upload/docs/application/pdf/2013-08/vectarion_ct_12442.pdf (accessed on 18/11/18). [158] Esnault P, Hraiech S, Bordes J, Forel J M, Adda M, Rambaud R, et al. (2019) Evaluation of Almitrine Infusion During Veno-Venous Extracorporeal Membrane Oxygenation for Severe Acute Respiratory Distress Syndrome in Adults. Anesthesia & Analgesia, 129(2), e48-e51. [159] European Medicines Agency (23 May 2013) Assessment report for almitrine-containing medicinal products for oral use. http://www.ema.europa.eu/docs/en_G B/document_library/Referrals_document/Almitrine/Recommendati on_provided_by_Pharmacovigilance_Risk_Assessment_Committee/WC500144134.pdf (accessed on 12/07/17). [160] Michard F, Wolff M A, Herman B, Wysocki M (2001) Right ventricular response to high-dose almitrine infusion in patients with severe hypoxemia related to acute respiratory distress syndrome. Critical care medicine. 29(1), 32-36. [161] Howard P (1989) Hypoxia, almitrine, and peripheral neuropathy. Thorax. 44(4):247. [162] Stavchansky S, Doluisio J T, MacLeod C M, Szalkowski M B, Bachand Jr R T, Heilman R, Sebree T B, Geary R S (1989) One year administration of almitrine bismesylate (Vectarion) to chronic obstructive pulmonary disease patients: pharmacokinetic analysis. Biopharmaceutics & drug disposition. 10(3):247-55. [163] MacLeod C N, Thomas R W, Bartley E A, Parkhurst G W, Bachand R T (1983) Effects and handling of almitrine bismesylate in healthy subjects. European journal of respiratory diseases. Supplement. 126:275-89. [164] Winkelmann B R, Kullmer T H, Kneissl D G, Trenk D, Kronenberger H (1994) Low-dose almitrine bismesylate in the treatment of hypoxemia due to chronic obstructive pulmonary disease. Chest. 105(5):1383-91. [165] Weitzenblum E, Schrijen F, Apprill M, Prefaut C, Yernault J C (1991) One year treatment with almitrine improves hypoxaemia but does not increase pulmonary artery pressure in COPD patients. European Respiratory Journal. 4(10):1215-22. [166] Bell R C, Mullins R C, West L G, Bachand R T, Johanson W G (1986) The effect of almitrine bismesylate on hypoxemia in chronic obstructive pulmonary disease. Annals of internal medicine. 105(3):342-6. [167] Bromet N, Singlas E (1984) Clinical pharmacokinetics of almitrine dimesylate. Presse medicale (Paris, France: 1983). (34):2071-7. [168] Górecka D, Sliwinski P, Palasiewicz G, Pachocki R, Zielinski J (2003) Effects of almitrine bismesylate on arterial blood gases in patients with chronic obstructive pulmonary disease and moderate hypoxaemia: a multicentre, randomised, double-blind, placebo-controlled study. Respiration. 70(3):275-83. [169] Barker H E, Paget J T, Khan A A, Harrington K J (2015) The tumour microenvironment after radiotherapy: mechanisms of resistance and recurrence. Nature reviews Cancer. 15(7):409. [170] Zhou Y, Hileman E O, Plunkett W, Keating M J, Huang P (2003) Free radical stress in chronic lymphocytic leukemia cells and its role in cellular sensitivity to ROS-generating anticancer agents. Blood. 101(10):4098-104. [171] Wondrak G T (2009) Redox-directed cancer therapeutics: molecular mechanisms and opportunities. Antioxidants & redox signaling. 11(12):3013-69. [172] Berndtsson M, Hägg M, Panaretakis T, Havelka A M, Shoshan M C, Linder S (2007) Acute apoptosis by cisplatin requires induction of reactive oxygen species but is not associated with damage to nuclear DNA. International journal of cancer. 120(1):175-80. [173] Sullivan R, Pare G C, Frederiksen L J, Semenza G L, Graham C H (2008) Hypoxia-induced resistance to anticancer drugs is associated with decreased senescence and requires hypoxia-inducible factor-1 activity. Molecular cancer therapeutics. 7(7):1961-73. [174] Doktorova H, Hrabeta J, Khalil M A, Eckschlager T (2015) Hypoxia-induced chemoresistance in cancer cells: The role of not only HIF-1. Biomedical Papers of the Medical Faculty of Palacky University in Olomouc. 159(2). [175] Saadjian A Y, Philip-Joel F F, Barret A, Levy S, Arnaud A G (1994) Effect of almitrine bismesylate on pulmonary vasoreactivity to hypoxia in chronic obstructive pulmonary disease. European Respiratory Journal. 7(5):862-8. [176] Prost J F, Desche P, Jardin F, Margairaz A (1991) Comparison of the effects of intravenous almitrine and positive end-expiratory pressure on pulmonary gas exchange in adult respiratory distress syndrome. European Respiratory Journal. 4(6):683-7. [177] Gallart L, Lu Q I, Puybasset L, Umamaheswara Rao G S, Coriat P, Rouby J J, NO Almitrine Study Group (1998) Intravenous almitrine combined with inhaled nitric oxide for acute respiratory distress syndrome. American journal of respiratory and critical care medicine. 158(6):1770-7. [178] Jolliet P, Bulpa P, Ritz M, Ricou B, Lopez J, Chevrolet J C (1997) Additive beneficial effects of the prone position, nitric oxide, and almitrine bismesylate on gas exchange and oxygen transport in acute respiratory distress syndrome. Critical care medicine. 25(5):786-94. [179] Silva-Costa-Gomes T, Gallart L, Valles J, Trillo L, Minguella J, Puig M M (2005) Low-vs high-dose almitrine combined with nitric oxide to prevent hypoxia during open-chest one-lung ventilation. British journal of anaesthesia. 95(3):410-6. [180] Parotte P, Boileau S, Marchand G, Laxenaire M C (1980) Effects of a respiratory analeptic with peripheral action-almitrine on ventilation immediately after surgery. In Annales de l'anesthesiologie francaise. 21 (5): 519-524. [181] Laxenaire M C, Borgo J, Comet C, Torrens J M (1980) Almitrine in the treatment of post-anaesthetic hypoventilation. Review Frangais Maladies Respiratoires. 8:225-33. [182] Stavchansky S, Doluisio J T, MacLeod C M, Sebree T B, Heilman R, Bachand Jr R T, Szalkowski M B, Geary R S (1989) Relative bioavailability of almitrine bismesylate in humans. Biopharmaceutics & drug disposition. 10(3):239-46. [183] Payen D, Muret J, Beloucif S, Gatecel C, Kermarrec N, Guinard N, Mateo J (1998) Inhaled nitric oxide, almitrine infusion, or their coadministration as a treatment of severe hypoxemic focal lung lesions. Anesthesiology: The Journal of the American Society of Anesthesiologists. 89(5):1157-65. [184] Amundson S A, Do K T, Vinikoor L C, Lee R A, Koch-Paiz C A, Ahn J, Reimers M, Chen Y, Scudiero D A, Weinstein J N, Trent J M (2008) Integrating global gene expression and radiation survival parameters across the 60 cell lines of the National Cancer Institute Anticancer Drug Screen. Cancer research. 68(2):415-24. [185] Polavarapu S, Mani A M, Gundala N K, Hari A D, Bathina S, Das U N (2014) Effect of polyunsaturated fatty acids and their metabolites on bleomycin-induced cytotoxic action on human neuroblastoma cells in vitro. PloS one. 9(12):e114766. [186] Dilly S J, Clark A J, Marsh A, Mitchell D A, Cain R, Fishwick C W, Taylor P C (2017) A chemical genomics approach to drug reprofiling in oncology: Antipsychotic drug risperidone as a potential adenocarcinoma treatment. Cancer letters. 393:16-21. [187] Yu C, Li Y, Holmes A, Szafranski K, Faulkes C G, Coen C W, Buffenstein R, Platzer M, De Magalhães J P, Church G M (2011) RNA sequencing reveals differential expression of mitochondrial and oxidation reduction genes in the long-lived naked mole-rat when compared to mice. PloS one. 6(11):e26729. [188] Lebowitz M S, Pedersen P L (1996) Protein inhibitor of mitochondrial ATP synthase: relationship of inhibitor structure to pH-dependent regulation. Archives of Biochemistry and Biophysics. 330(2):342-354. [189] Gordon-Smith D J, Carbajo R J, Yang J C, Videler H, Runswick M J, Walker J E, Neuhaus D (2001). Solution structure of a C-terminal coiled-coil domain from bovine IF1: The inhibitor protein of F1 ATPase. Journal of molecular biology, 308(2), 325-339. [190] Cabezon E, Butler P J G, Runswick M J, Walker J E (2000) Modulation of the oligomerization state of the bovine F1-ATPase inhibitor protein, IF1, by pH. Journal of Biological Chemistry. 275(33):25460-25464. [191] Bason J V, Runswick M J, Fearnley I M, Walker J E (2011) Binding of the inhibitor protein IF₁ to bovine F₁-ATPase. Journal of molecular biology. 406(3):443-453. [192] Rothbard J B, Garlington S, Lin Q, Kirschberg T, Kreider E, McGrane P L, . . . & Khavari P A (2000) Conjugation of arginine oligomers to cyclosporin A facilitates topical delivery and inhibition of inflammation. Nature medicine. 6(11):1253-1257. [193] Mix K A, Lomax J E, Raines R T (2017) Cytosolic delivery of proteins by bioreversible esterification. Journal of the American Chemical Society. 139(41):14396-14398. [194] Ressler V T, Mix K A, Raines R T (2019) Esterification delivers a functional enzyme into a human cell. ACS chemical biology. 14(4):599-602. [195] Zorzi A, Middendorp S J, Wilbs J, Deyle K, Heinis C (2017) Acylated heptapeptide binds albumin with high affinity and application as tag furnishes long-acting peptides. Nature communications. 8(1):1-9. [196] Ekrami H M, Kennedy A R, Shen W C (1995) Water-soluble fatty acid derivatives as acylating agents for reversible lipidization of polypeptides. FEBS letters. 371(3):283-286. [197] Gautam A, Singh H, Tyagi A, Chaudhary K, Kumar R, Kapoor P, Raghava G P (2012) CPPsite: a curated database of cell penetrating peptides. Database. 2012. [198] Váquez J, Sun C, Du J, Fuentes L, Sumners C, Raizada M K (2003) Transduction of a functional domain of the AT1 receptor in neurons by HIV-Tat PTD. Hypertension. 41(3):751-756. [199] Tünnemann G, Martin R M, Haupt S, Patsch C, Edenhofer F, Cardoso M C (2006) Cargo-dependent mode of uptake and bioavailability of TAT-containing proteins and peptides in living cells. The FASEB journal. 20(11):1775-1784. [200] Vivés E, Schmidt J, Pélegrin A (2008) Cell-penetrating and cell-targeting peptides in drug delivery. Biochimica et Biophysica Acta (BBA)-Reviews on Cancer. 1786(2):126-138. [201] Begley R, Liron T, Baryza J, Mochly-Rosen D (2004) Biodistribution of intracellularly acting peptides conjugated reversibly to Tat. Biochemical and biophysical research communications. 318(4): 949-954. [202] Miyaji Y, Walter S, Chen L, Kurihara A, Ishizuka T, Saito M, . . . Okazaki O (2011) Distribution of KAI-9803, a novel δ-protein kinase C inhibitor, after intravenous administration to rats. Drug metabolism and disposition. 39(10):1946-1953. [203] Borsello T, Clarke P G, Hirt L, Vercelli A, Repici M, Schorderet D F, . . . Bonny C (2003) A peptide inhibitor of c-Jun N-terminal kinase protects against excitotoxicity and cerebral ischemia. Nature medicine. 9(9):1180-1186. [204] Flynn C R, Cheung-Flynn J, Smoke C C, Lowry D, Roberson R, Sheller M R, Brophy C M (2010) Internalization and intracellular trafficking of a PTD-conjugated anti-fibrotic peptide, AZX100, in human dermal keloid fibroblasts. Journal of pharmaceutical sciences. 99(7):3100-3121. [205] Chen L, Wright L R, Chen C H, Oliver S F, Wender P A, Mochly-Rosen D (2001) Molecular transporters for peptides: delivery of a cardioprotective EPKC agonist peptide into cells and intact ischemic heart using a transport system, R7. Chemistry & biology. 8(12):1123-1129. [206] Mitchell D J, Steinman L, Kim D T, Fathman C G, Rothbard J B (2000) Polyarginine enters cells more efficiently than other polycationic homopolymers. The Journal of Peptide Research. 56(5):318-325. [207] Brewer S J, Sassenfeld H M (1985) The purification of recombinant proteins using C-terminal polyarginine fusions. Trends in Biotechnology. 3(5):119-122. [208] Holm T, Räägel H, Andaloussi S E, Hein M, Mäe M, Pooga M, Langel Ü (2011) Retro-inversion of certain cell-penetrating peptides causes severe cellular toxicity. Biochimica et Biophysica Acta (BBA)-Biomembranes. 1808(6):1544-1551. [209] Brugidou J, Legrand C, Mery J, Rabie A (1995) The retro-inverso form of a homeobox-derived short peptide is rapidly internalized by cultured neurons: a new basis for an efficient intracellular delivery system. Biochemical and biophysical research communications. 214(2):685-693. [210] Nasrollahi S A, Taghibiglou C, Azizi E, Farboud E S (2012) Cell-penetrating peptides as a novel transdermal drug delivery system. Chemical biology & drug design. 80(5):639-646. [211] Jiang T, Olson E S, Nguyen Q T, Roy M, Jennings P A, Tsien R Y (2004) Tumor imaging by means of proteolytic activation of cell-penetrating peptides. Proceedings of the National Academy of Sciences. 101(51):17867-17872. [212] Savariar E N, Felsen C N, Nashi N, Jiang T, Ellies L G, Steinbach P, . . . Nguyen Q T (2013) Real-time in vivo molecular detection of primary tumors and metastases with ratiometric activatable cell-penetrating peptides. Cancer research. 73(2):855-864. [213] Unkart J T, Chen S L, Wapnir I L, GonzAlez J E, Harootunian A, Wallace A M (2017) Intraoperative tumor detection using a ratiometric activatable fluorescent peptide: a first-in-human phase 1 study. Annals of surgical oncology. 24(11):3167-3173. [214] Futaki S, Ohashi W, Suzuki T, Niwa M, Tanaka S, Ueda K, . . . & Sugiura Y (2001) Stearylated arginine-rich peptides: a new class of transfection systems. Bioconjugate chemistry. 12(6):1005-1011. [215] Katayama S, Hirose H, Takayama K, Nakase I, Futaki S (2011) Acylation of octaarginine: Implication to the use of intracellular delivery vectors. Journal of controlled release. 149(1):29-35. [216] Taylor B N, Mehta R R, Yamada T, Lekmine F, Christov K, Chakrabarty A M, . . . Gupta T K D. (2009) Noncationic peptides obtained from azurin preferentially enter cancer cells. Cancer research. 69(2):537-546. [217] Kang Y C, Son M, Kang S, Im S, Piao Y, Lim K S, . . . Pak Y K (2018) Cell-penetrating artificial mitochondria-targeting peptide-conjugated metallothionein 1A alleviates mitochondrial damage in Parkinson's disease models. Experimental & molecular medicine. 50(8):1-13. [218] Horton K L, Stewart K M, Fonseca S B, Guo Q, Kelley S O (2008) Mitochondria-penetrating peptides. Chemistry & biology. 15(4):375-82. [219] Jean S R, Ahmed M, Lei E K, Wisnovsky S P, Kelley S O (2016) Peptide-mediated delivery of chemical probes and therapeutics to mitochondria. Accounts of chemical research. 49(9):1893-902. [220] Qian Z, Liu T, Liu Y Y, Briesewitz R, Barrios A M, Jhiang S M, Pei D (2012) Efficient delivery of cyclic peptides into mammalian cells with short sequence motifs. ACS chemical biology. 8(2):423-31. [221] Qian Z, Martyna A, Hard R L, Wang J, Appiah-Kubi G, Coss C, Phelps M A, Rossman J S, Pei D (2016) Discovery and mechanism of highly efficient cyclic cell-penetrating peptides. Biochemistry. 55(18):2601-12. [222] Appiah Kubi G, Qian Z, Amiar S, Sahni A, Stahelin R V, Pei D (2018) Non-Peptidic Cell-Penetrating Motifs for Mitochondrion-Specific Cargo Delivery. Angewandte Chemie. 130(52):17429-34. [223] Zielonka J, Joseph J, Sikora A, Hardy M, Ouari O, Vasquez-Vivar J, Cheng G, Lopez M, Kalyanaraman B (2017) Mitochondria-targeted triphenylphosphonium-based compounds: syntheses, mechanisms of action, and therapeutic and diagnostic applications. Chemical reviews. 117(15):10043-120. [224] Pathak R K, Marrache S, Ham D A, Dhar S (2014) Mito-DCA: a mitochondria targeted molecular scaffold for efficacious delivery of metabolic modulator dichloroacetate. ACS chemical biology. 9(5):1178-87. [225] Appiah Kubi G, Qian Z, Amiar S, Sahni A, Stahelin R V, Pei D (2018) Non-Peptidic Cell-Penetrating Motifs for Mitochondrion-Specific Cargo Delivery. Angewandte Chemie. 130(52):17429-17434. [226] Heinis C, Rutherford T, Freund S, Winter G (2009) Phage-encoded combinatorial chemical libraries based on bicyclic peptides. Nature chemical biology. 5(7):502. [227] Baeriswyl V, Rapley H, Pollaro L, Stace C, Teufel D, Walker E, Chen S, Winter G, Tite J, Heinis C (2012) Bicyclic peptides with optimized ring size inhibit human plasma kallikrein and its orthologues while sparing paralogous proteases. Chem Med Chem. 7(7):1173-6. [228] Angelini A, Cendron L, Chen S, Touati J, Winter G, Zanotti G, Heinis C (2012) Bicyclic peptide inhibitor reveals large contact interface with a protease target. ACS chemical biology. 7(5):817-21. [229] Teufel D P, Bennett G, Harrison H, Van Rietschoten K, Pavan S, Stace C, Le Floch F, Van Bergen T, Vermassen E, Barbeaux P, Hu T T (2018) Stable and long-lasting, novel bicyclic peptide plasma kallikrein inhibitors for the treatment of diabetic macular edema. Journal of medicinal chemistry. 61(7):2823-36. [230] Eder M, Pavan S, Bauder-Wüst U, van Rietschoten K, Baranski A C, Harrison H, Campbell S, Stace C L, Walker E H, Chen L, Bennett G (2019) Bicyclic peptides as a new modality for imaging and targeting of proteins overexpressed by tumors. Cancer research. 79(4):841-52. 1231 ] Rhodes C A, Pei D (2017) Bicyclic Peptides as Next-Generation Therapeutics. Chemistry-A European Journal. 23(52):12690-703. [232] Lian W, Jiang B, Qian Z, Pei D (2014) Cell-permeable bicyclic peptide inhibitors against intracellular proteins. Journal of the American Chemical Society. 136(28):9830-3. [233] Qian Z, Rhodes C A, McCroskey L C, Wen J, Appiah-Kubi G, Wang D J, Guttridge D C, Pei D (2017) Enhancing the cell permeability and metabolic stability of peptidyl drugs by reversible bicyclization. Angewandte Chemie International Edition. 56(6):1525-9. [234] Satre M, De Jerphanion M B, Huet J, Vignais P V (1975) ATPase inhibitor from yeast mitochondria. Purification and properties. Biochimica et Biophysica Acta (BBA)-Bioenergetics. 387(2):241-55. [235] Yampolsky L Y, Stoltzfus A (2005) The exchangeability of amino acids in proteins. Genetics. 170(4):1459-1472. [236] Wender P A, Mitchell D J, Pattabiraman K, Pelkey E T, Steinman L, Rothbard J B (2000) The design, synthesis, and evaluation of molecules that enable or enhance cellular uptake: peptoid molecular transporters. Proceedings of the National Academy of Sciences. 97(24):13003-13008. [237] Naso M F, Tomkowicz B, Perry W L, Strohl W R (2017) Adeno-associated virus (AAV) as a vector for gene therapy. BioDrugs. 31(4):317-34. [238] Whittemore K, Derevyanko A, Martinez P, Serrano R, Pumarola M, Bosch F, Blasco M A (2019) Telomerase gene therapy ameliorates the effects of neurodegeneration associated to short telomeres in mice. Aging (Albany NY). 11(10):3280. [239] Lee Y W, Luther D C, Kretzmann J A, Burden A, Jeon T, Zhai S, Rotello V M (2019) Protein delivery into the cell cytosol using non-viral nanocarriers. Theranostics. 9 (11):3280-3292. [240] Chatin B, Mével M, Devalliëre J, Dallet L, Haudebourg T, Peuziat P, Colombani T, Berchel M, Lambert O, Edelman A, Pitard B (2015) Liposome-based formulation for intracellular delivery of functional proteins. Mol. Ther.-Nucleic Acids 4, No. e244. [241] Shi B, Keough E, Matter A, Leander K, Young S, Carlini E, Sachs A B, Tao W, Abrams M, Howell B, Sepp-Lorenzino L (2011) Biodistribution of small interfering RNA at the organ and cellular levels after lipid nanoparticle-mediated delivery. J. Histochem. Cytochem. 59(8):727-740. [242] Tillack K, Aboutalebi H, Kramer E R (2015) An efficient and versatile system for visualization and genetic modification of dopaminergic neurons in transgenic mice. PloS one. 10(8):e0136203. [243] Pickrell A M, Pinto M, Hida A, Moraes C T (2011) Striatal dysfunctions associated with mitochondrial DNA damage in dopaminergic neurons in a mouse model of Parkinson's disease. Journal of Neuroscience. 31(48):17649-58. [244] Chen L, Xie Z, Turkson S, Zhuang X (2015) A53T human α-synuclein overexpression in transgenic mice induces pervasive mitochondria macroautophagy defects preceding dopamine neuron degeneration. Journal of Neuroscience. 35(3):890-905. [245] Schönig K, Freundlieb S, Gossen M (2013) Tet-Transgenic Rodents: a comprehensive, up-to date database. Transgenic research. 22(2):251-4. [246] Premsrirut P K, Dow L E, Kim S Y, Camiolo M, Malone C D, Miething C, Scuoppo C, Zuber J, Dickins R A, Kogan S C, Shroyer K R (2011) A rapid and scalable system for studying gene function in mice using conditional RNA interference. Cell. 145(1):145-58. [247] Zinn E, Pacouret S, Khaychuk V, Turunen H T, Carvalho L S, Andres-Mateos E, Shah S, Shelke R, Maurer A C, Plovie E, Xiao R (2015) In silico reconstruction of the viral evolutionary lineage yields a potent gene therapy vector. Cell reports. 12(6):1056-68. [248] Ginn S L, Amaya A K, Alexander I E, Edelstein M, Abedi M R (2018) Gene therapy clinical trials worldwide to 2017: An update. The journal of gene medicine. 20(5):e3015. [249] Coune P G, Schneider B L, Aebischer P (2012) Parkinson's disease: gene therapies. Cold Spring Harbor perspectives in medicine. a009431. [250] Feng L R, Maguire-Zeiss K A (2010) Gene Therapy in Parkinson's Disease. CNS drugs. 24(3):177-92. [251] Forsayeth J, Bankiewicz K S, Aminoff M J (2010) Gene therapy for Parkinson's disease: where are we now and where are we going?Expert review of neurotherapeutics. 10(12):1839-45. [252] Axelsen T M, Woldbye D P (2018) Gene therapy for Parkinson's disease, an update. Journal of Parkinson's disease. 1-21. [253] Palfi S, Gurruchaga J M, Ralph G S, Lepetit H, Lavisse S, Buttery P C, Watts C, Miskin J, Kelleher M, Deeley S, Iwamuro H (2014) Long-term safety and tolerability of ProSavin, a lentiviral vector-based gene therapy for Parkinson's disease: a dose escalation, open-label, phase 1/2 trial. The Lancet. 383(9923):1138-46. [254] LeWitt P A, Rezai A R, Leehey M A, Ojemann S G, Flaherty A W, Eskandar E N, Kostyk S K, Thomas K, Sarkar A, Siddiqui M S, Tatter S B (2011) AAV2-GAD gene therapy for advanced Parkinson's disease: a double-blind, sham-surgery controlled, randomised trial. The Lancet Neurology. 10(4):309-19. [255] Schriner S E, Linford N J, Martin G M, Treuting P, Ogburn C E, Emond M, Coskun P E, Ladiges W, Wolf N, Van Remmen H, Wallace D C (2005) Extension of murine life span by overexpression of catalase targeted to mitochondria. Science. 308(5730):1909-11. [256] Mitsui A, Hamuro J, Nakamura H, Kondo N, Hirabayashi Y, Ishizaki-Koizumi S, Hirakawa T, Inoue T, Yodoi J. Overexpression of human thioredoxin in transgenic mice controls oxidative stress and life span. Antioxidants and Redox Signaling. 2002 Aug. 1; 4(4):693-6. [257] Owen S C, Doak A K, Wassam P, Shoichet M S, Shoichet B K (2012) Colloidal aggregation affects the efficacy of anticancer drugs in cell culture. ACS chemical biology. 7(8):1429-35. [258] Owen S C, Doak A K, Ganesh A N, Nedyalkova L, McLaughlin C K, Shoichet B K, Shoichet M S (2014) Colloidal drug formulations can explain “bell-shaped” concentration-response curves. ACS chemical biology. 9(3):777-84.

1. A cosmetic composition comprising (or consisting of) at least one [any] IF1 protein/fragment (or sequence variant thereof, including partially/completely retroinverse sequence thereof), and/or fusion protein(s) thereof (optionally comprising at least one Cell Penetrating Peptide [CPP] sequence, optionally a Tat and/or poly-arginine sequence, optionally partially/completely retroinverse sequence thereof), optionally lipidated (i.e. with at least one covalently bound lipidic/lipid moiety, optionally at least one fatty acid [optionally acylated to its N-terminus] e.g. [non-limiting] a myristoyl/palmitoyl/stearoyl group), optionally modified at its N-(non-limiting e.g. acylated [non-limiting e.g. acetylated]) and/or C-terminal (non-limiting e.g. amidated) ends, optionally wherein one or more amino-acids in the sequence are corresponding D-amino acids, optionally wherein one or more of its carboxyl groups are esterified, and/or at least one cosmetically acceptable salt, solvate, hydrate, prodrug, liposome, nanoparticle (e.g. lipid nanoparticle, LNP) or other vector of the art thereof;
optionally a cosmetic composition comprising (or consisting of) at least one peptide/protein (optionally with one or more of its carboxyl groups esterified) comprising (or consisting of) [preferably wherein the following is in N to C-terminal order] at least one Cell Penetrating Peptide sequence (CPP, e.g. a poly-arginine CPP, optionally with a fatty acid [e.g. of between 2 to 25 carbons] acylated to its N-terminal end) conjoined with (e.g. peptide bonded to) at least one Mitochondrial Import Sequence (MIS; conferring mitochondrial matrix localization, optionally/preferably wherein the MIS is that used by the species administered to for its native IF1 protein; e.g. MIS that human uses for its native IF1 protein) conjoined with (e.g. peptide bonded to) at least one “mature” (without MIS) IF1 protein/fragment (or sequence variant thereof) that is optionally/preferably an IF1 protein sufficiently truncated at its C-terminal end (e.g. truncated up to {using “mature” [without MIS] IF1 protein numbering} its 60^{th} or 47^{th} residue), and/or optionally truncated at its N-terminal end (e.g. by any number of residues up to 9 [or 13] residues), and/or with one or more amino acid substitutions in its “phosphorylation control switch” and/or “pH dependence motif” (e.g. one or more of {using “mature” [without MIS] IF1 protein numbering} S14A [or T14A], E26A [or Q26A or E26Q], H48A [or Y48A], H49K [or H49A or H49R], H55A [or Y55A or V55A], H56A [or T56A or S56A] substitutions), such that it can still inhibit F₁F₀ ATP hydrolysis but it cannot (or cannot as readily) form IF1 protein tetramers (and higher oligomers) at alkaline pH, preferably such that it can more potently inhibit F₁F₀ ATP hydrolysis at the normal, alkaline pH (˜pH 8) of the mitochondrial matrix (than native/unmodified IF1 protein), optionally/preferably wherein this IF1 protein/fragment (or sequence variant thereof) has a sequence derived/modified from the IF1 protein sequence of the species to be administered to [e.g. human] or a species with a longer maximal lifespan, optionally a species with a very long maximal lifespan such as a whale, e.g. bowhead or blue whale, and/or at least one cosmetically acceptable salt, solvate, hydrate, prodrug, liposome, nanoparticle (e.g. lipid nanoparticle, LNP) or other vector of the art thereof;
optionally a cosmetic composition comprising (or consisting of) at least one peptide/protein comprising (or consisting of) at least one sequence that is a designed concatenation of sequences that are each naturally occurring in the human body, e.g, wherein the CPP component is SEQ ID NO:455 (or SEQ ID NO:461, or residues 4-11 of SEQ ID NO:453), the MIS component is SEQ ID NO:162, and the IF1 protein/fragment sequence component is from human IF1 protein (non-limiting e.g. using “mature” [without MIS] IF1 protein numbering: residues: 1-60, 10-60, 14-60, 13-47, 14-47, 42-58, which are the amino acid sequences encoded by DNA sequences SEQ ID NO:1473, SEQ ID NO:1476, SEQ ID NO:1479, SEQ ID NO:1482, SEQ ID NO:1485, SEQ ID NO:1488 respectively), and/or at least one cosmetically acceptable salt, solvate, hydrate, prodrug, liposome, nanoparticle (e.g. lipid nanoparticle, LNP) or other vector of the art thereof;
optionally a cosmetic composition comprising (or consisting of) at least one peptide/protein comprising (or consisting of) at least one sequence selected from SEQ ID NO:166 to SEQ ID NO:438, and/or at least one fragment thereof (for non-limiting example wherein the epitope/affinity tag component [if present] is absent, and/or the Cell Penetrating Peptide component [if present] is absent), and/or concatenated fragments thereof, and/or a functional (can inhibit/reduce F₁F₀ ATP hydrolysis in a cell and/or in a Sub-Mitochondrial Particle [SMP] assay of F₁F₀ ATP hydrolysis) sequence variant(s) thereof (optionally produced by conservative substitution[s]), and/or at least one cosmetically acceptable salt, solvate, hydrate, prodrug, liposome, nanoparticle (e.g. lipid nanoparticle, LNP) or other vector of the art thereof;
preferably in a form suitable for application to the skin/scalp of a subject (preferably human), optionally their face;
preferably wherein the composition contains at least one cosmetically/dermatologically acceptable carrier;
optionally wherein the composition contains at least one further active agent, which can perform one or more of skin/scalp care/protection/treatment/repair/beautification/cleaning/fragrance/cosmetic purpose/appearance change, optionally for human skin/scalp, wherein a number of such agents are known in the art (non-limiting e.g. retinol and other retinoids).

2. A cosmetic composition according to claim 1 in at least one form/formulation selected from a group comprising (or consisting of): gel, emulsion, oil/water emulsion, water/oil emulsion, milk, lotion, ointment, stick, pencil, spray, cream, cream gel, multiple emulsion, anhydrous composition, aqueous dispersion, oil, balsam, foam, hydroalcoholic solution, hydroglycolic solution, hydrogel, liniment, sera, serum, mousse, pomade, powder, bar, aerosol, granule, solution, suspension, emulsion, syrup, polysaccharide film, jelly, gelatin, emollient lotion, emollient milk, emollient cream, emulsion of oil and/or silicone in water, emulsion of water in oil and/or silicone, balm, liquid, paste, aerosol, butter,
and/or incorporated into a product for administration/application to the human skin/scalp, optionally selected from a group comprising (or consisting of): cosmetic product for use upon the skin/scalp, cosmetic product for use upon the face, daily use skin care product, exfoliant, skin smoothing product, product for improving/smoothing skin texture, anti-aging/anti-wrinkle skin product/cream/serum, anti-hair loss product, hair growth promoting product, anti-hair greying product, hair dye, skin cream, face cream, eye cream, anti-acne/spot cream, moisterizer, cleanser, shampoo, conditioner, anti-dandruff product, soap, shower gel, scalp lotion, body oil, skin/body/face scrub, milk/cream for care of skin and/or hair, cleansing cream, foundation tint base, sunscreen/sunblock/sun cream (e.g. offering protection against UVA and/or UVB radiation), fake sun tan product, skin darkening product, skin whitening product/cream, shaving cream/foam/balm, perfume, aftershave, deodorant, anti-persperant, make-up product, lip rouge, lipstick, lip gloss, lip protector, mascara, nail varnish, concealer, under-eye concealer, blusher, mascara, make-up foundation, foundation, BB cream or CC cream or DD cream or similar, make-up removing product/lotion/milk/cream, eye shadow, unguent, anti-cellulite product, anti-stretch mark product, anti-varicose vein product, daily peel, face mask, eye mask, night mask, sleeping mask, toothpaste, mouthwash,
and/or incorporated/absorbed/adsorbed into one or more of a fabric, non-woven fabric, textile, a material used for clothing, garment, natural or synthetic fibre, wool, face mask, sleeping mask, eye mask, plaster, medical device, bandage, gauze, wipe, patch, adhesive skin patch, non-adhesive skin patch, microelectric patch, towelette, hydrogel,
and/or adsorbed on at least one cosmetically/pharmaceutically acceptable solid organic polymer or solid mineral support selected from the group comprising (or consisting of) talc, bentonite, silica, starch, maltodextrin or inorganic carrier, adsorbed on powdered organic and/or inorganic polymers.

3. At least one cosmetic composition according to one or more of claims 1-2 for use as a cosmetic, wherein an amount (preferably an effective amount e.g. a cosmetically effective amount) is administered to a subject, optionally wherein the subject self-administers,
preferably administered to the subject's skin/scalp, optionally their face, preferably wherein the subject is a human.

4. At least one cosmetic composition according to one or more of claims 1-2 for use in reducing/slowing/delaying/preventing/eliminating one or more visible signs of aging wherein it is administered to one or more areas of human skin (optionally already showing one or more signs of aging), preferably wherein said composition is applied at least once per day for a time period sufficient to provide an elimination/reduction/slowing/delay/prevention in the visible signs of aging of that portion of human skin, wherein said time period is at least 2 weeks.

5. An “immature” (with Mitochondrial Import Sequence, MIS) or “mature” (without MIS) IF1 protein/fragment (or sequence variant thereof), or fusion protein thereof, wherein one or more of the following applies to (is true of) part(s) or all of it (wherein all possible combinations are contemplated {including all possible combinations of elements/descriptors within, and across, different bullet points} except those that are mutually exclusive):
(i) produced/isolated/purified/substantially purified/partially purified;
(ii) associated with a pharmaceutically/cosmetically acceptable salt[s];
(iii) IF1 protein/fragment (or sequence variant thereof) component/entirety comprises (or consists of) bowhead whale (Balaena mysticetus) IF1 protein;
(iv) IF1 protein/fragment (or sequence variant thereof) component/entirety comprises (or consists of) blue whale (Balaenoptera musculus) IF1 protein;
(v) one or more (or two/three/four/five/six/seven/eight/nine/ten/eleven/twelve/thirteen/fourteen/fifteen/sixteen/seventeen or more) of the following is true of the IF1 protein/fragment (or sequence variant thereof) component/entirety: (using “mature” [without Mitochondrial Import Sequence, MIS] IF1 protein numbering): 49^{th} residue is not histidine, 14^{th} residue is not a residue that can be phosphorylated (i.e. is not serine or threonine), 26^{th} residue is not glutamic acid, 48^{th} residue is not histidine, 55^{th} residue is not histidine, 56^{th} residue is not histidine, 49^{th} residue is lysine or alanine or arginine, 14^{th} residue is alanine, 26^{th} residue is alanine or glutamine, 48^{th} residue is alanine, 55^{th} residue is alanine, 56^{th} residue is alanine, 79^{th} residue is glycine or asparagine, 76^{th} residue is lysine, 73^{rd} residue is serine, 62^{nd} residue is histidine, 82^{nd} residue is aspartic acid, 83^{rd} residue is aspartic acid, 84^{th} residue is aspartic acid, 85^{th} residue is aspartic acid, 57^{th} residue is valine, 54^{th} residue is serine or aspartic acid, 61^{st} residue is glutamine, 51^{st} residue is asparagine, 47^{th} residue is glutamic acid, 46^{th} residue is arginine, 44^{th} residue is serine, 39^{th} residue is lysine, 38^{th} residue is alanine or glutamic acid, 37^{th} residue is arginine or cysteine or lysine, 36^{th} residue is aspartic acid or glutamic acid, 29^{th} residue is histidine, 27^{th} residue is alanine, 25^{th} residue is lysine, 17^{th} residue is aspartic acid, 12^{th} residue is glycine, 11^{th} residue is serine or threonine, 10^{th} residue is serine or glycine, 9^{th} residue is glycine, 8^{th} residue is leucine or glycine, 6^{th} residue is aspartic acid or glycine, 5^{th} residue is alanine, 4^{th} residue is serine or glycine, 3^{rd} residue is glutamic acid or serine or lysine, 2nd residue is glycine, 1^{st} residue is leucine, wherein in particular sub-embodiments (wherein all possible combinations are contemplated, except if mutually exclusive):
(a) three (3) or more of the list is true;
(b) five (5) or more of the list is true;
(c) seven (7) or more of the list is true;
(d) nine (9) or more of the list is true;
(e) eleven (11) or more of the list is true;
(f) thirteen (12) or more of the list is true;
(g) fourteen (14) or more of the list is true;
(h) fifteen (15) or more of the list is true;
(i) sixteen (16) or more of the list is true;
(j) seventeen (17) or more of the list is true;
(k) at least one IF1 protein/fragment sequence variant comprises (or consists of) the IF1 protein/fragment sequence of human (or other species with a long maximal lifespan, optionally a species with a longer maximal lifespan than human) with one or more substitutions (and/or addition of one or more aspartic acid residues to its C-terminal end) to make one or more of the list be true;
(l) at least one IF1 protein/fragment sequence variant comprises (or consists of) the IF1 protein/fragment sequence of bowhead/blue whale with one or more substitutions (and/or addition of one or more aspartic acid residues to its C-terminal end) to make one or more of the list be true;
(vi) IF1 protein/fragment (or sequence variant thereof) component/entirety comprises (or consists of) a sequence variant of IF1 protein (preferably wherein one/two/three/four/five or more of the descriptors in bullet point (v) above apply to it) found in a long-lived species (high maximal lifespan), preferably which has an equal or greater maximal lifespan than Bos taurus, more preferably which has an equal or greater maximal lifespan than human, and more preferably which has a greater maximal lifespan than human e.g. bowhead or blue whale;
(vii) IF1 protein/fragment (or sequence variant thereof) component/entirety comprises (or consists of) an IF1 protein/fragment (optionally from mammal, Bos taurus, or human, or blue or bowhead whale) with one or more of {using “mature” [without MIS] IF1 protein numbering} S14A (or T14A), H49K (or H49A or H49R), E26A (or E26Q or Q26A), H48A (or Y48A), H55A (or Y55A), H56A (or T56A or S56A) substitutions, optionally also/instead with 1-3 (or 1-5) aspartic acid (D) residues added to its C-terminal end;
(viii) IF1 protein/fragment (or sequence variant thereof) component/entirety comprises (or consists of) human IF1 protein/fragment with one or more of {using “mature” [without MIS] IF1 protein numbering} S14A, H49K (or H49A or H49R), E26A (or E26Q), H48A, H55A, H56A substitutions, optionally also/instead with 1-3 (or 1-5) aspartic acid (D) residues added to its C-terminal end;
(ix) IF1 protein/fragment (or sequence variant thereof) component/entirety comprises (or consists of) blue whale IF1 protein/fragment with one or more of {using “mature” [without MIS] IF1 protein numbering} T14A, H49K (or H49A or H49R), E26A (or E26Q), H48A, H55A, H56A substitutions, optionally also/instead with 1-3 (or 1-5) aspartic acid (D) residues added to its C-terminal end;
(x) IF1 protein/fragment (or sequence variant thereof) component/entirety comprises (or consists of) bowhead whale IF1 protein/fragment with one or more of {using “mature” [without MIS] IF1 protein numbering} H49K (or H49A or H49R), E26A (or E26Q), H48A, H55A, H56A substitutions, optionally also/instead with 1-3 (or 1-5) aspartic acid (D) residues added to its C-terminal end;
(xi) IF1 protein/fragment (or sequence variant thereof) component/entirety comprises (or consists of) an IF1 protein fragment (using “mature” [without MIS] IF1 protein numbering) less than z amino acids long, wherein z is an integer selected from a group comprising 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 [different values of z are different embodiments];
(xii) IF1 protein/fragment (or sequence variant thereof) component/entirety comprises (or consists of) an IF1 protein fragment (using “mature” [without MIS] IF1 protein numbering) x-y, where x is an integer between 1 and 20 (or between 1 and 44, or between 1 and 84), and y is an integer between 40 and 85 (or between 40 and 85, or between 2 and 85) [different values of x and/or y are different embodiments; within their aforementioned range constraints, all possible combinations of x and y integer values are contemplated];
(xiii) IF1 protein/fragment (or sequence variant thereof) component/entirety comprises (or consists of) an IF1 protein fragment selected from a group comprising (using “mature” [without MIS] IF1 protein numbering): 1-84, 2-84, 3-84, 4-84, 5-84, 6-84, 7-84, 8-84, 9-84, 10-84, 11-84, 12-84, 13-84, 14-84, 15-84, 16-84, 17-84, 18-84, 19-84, 20-84, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 81-84, 82-84, 83-84, OR a sub-sequence/fragment of one of these aforementioned fragments;
(xiv) IF1 protein/fragment (or sequence variant thereof) component/entirety comprises (or consists of) an IF1 protein fragment selected from a group comprising: IF1 protein residues {using “mature” [without MIS] IF1 protein numbering}: 14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 14-60, 10-84, 14-84, 18-84, 10-50, 1-45, 42-56, 42-47, 48-56, 49-55;
(xv) IF1 protein/fragment (or sequence variant thereof) component/entirety comprises (or consists of) IF1 protein residues [preferably from a species with a maximal lifespan at least as long as Bos taurus, more preferably from a very long-lived mammal e.g. human, or (more preferred) blue or bowhead whale] (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably wherein, if the 14^{th} residue isn't alanine, it is substituted to be alanine;
(xvi) IF1 protein/fragment (or sequence variant thereof) component/entirety comprises (or consists of) human IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably with a S14A substitution;
(xvii) IF1 protein/fragment (or sequence variant thereof) component/entirety comprises (or consists of) whale (non-limiting e.g. selected from a group comprising bowhead, fin, blue, humpback, killer, sperm, gray, Cuvier's beaked, long-finned pilot whale) IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably wherein, if the 14^{th} residue isn't alanine, it is substituted to be so;
(xviii) IF1 protein/fragment (or sequence variant thereof) component/entirety comprises (or consists of) blue whale IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, preferably with a T14A substitution;
(xix) IF1 protein/fragment (or sequence variant thereof) component/entirety comprises (or consists of) bowhead whale IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60;
(xx) IF1 protein/fragment (or sequence variant thereof) component/entirety comprises (or consists of) mammal/Bos Taurus/rodent/mouse/rat/rabbit IF1 protein residues (using “mature” [without MIS] IF1 protein numbering): one or more of (or sequence variant thereof) 10-47, 13-47, 14-47, 1-56, 1-57, 1-58, 1-59, 1-60, 10-56, 10-57, 10-58, 10-59, 10-60, 42-58, 42-59;
(xxi) IF1 protein/fragment (or sequence variant thereof) component/entirety comprises (or consists of) IF1 protein residues (using “mature” [without MIS] IF1 protein numbering): 42-58 or 42-59 (or sequence variant thereof);
(xxii) IF1 protein/fragment (or sequence variant thereof) component/entirety comprises (or consists of) human IF1 protein residues (using “mature” [without MIS] IF1 protein numbering): 42-58 or 42-59 (or sequence variant thereof), optionally with one or more of E51N, V54S, K57V substitutions;
(xxiii) IF1 protein/fragment (or sequence variant thereof) component/entirety comprises (or consists of) IF1 protein fragment shorter than 40 (or 35, or 30, or 25, or 20, or 15, or 10, or 5) amino acids, which contains H49 residue (or sequence variant thereof);
(xxiv) IF1 protein/fragment (or sequence variant thereof) component/entirety comprises (or consists of) short-living mammal (e.g. rodent, e.g. mouse) IF1 protein fragment shorter than 40 (or 35, or 30, or 25, or 20, or 15, or 10, or 5) amino acids, which contains H49 residue (or sequence variant thereof);
(xxv) IF1 protein/fragment (or sequence variant thereof) component/entirety comprises (or consists of) IF1 protein fragment (or sequence variant thereof) from a species that will be administered with the peptide/protein (and/or composition thereof);
(xxvi) IF1 protein/fragment (or sequence variant thereof) component/entirety comprises (or consists of) IF1 protein fragment (or sequence variant thereof) from a species with a maximal lifespan at least as long as Bos taurus, more preferably from a very long-lived mammal e.g. human, or (more preferred) blue or bowhead whale;
(xxvii) IF1 protein/fragment (or sequence variant thereof) component/entirety comprises (or consists of) IF1 protein fragment (or sequence variant thereof) from a mammal species with a maximal lifespan at least as short as mouse;
(xxviii) IF1 protein/fragment (or sequence variant thereof) component/entirety comprises (or consists of) married/combined fragment(s) and/or residue(s) of IF1 protein sequences from two or more different species, preferably wherein at least one of these species has a high maximal lifespan, preferably equal to or longer than human e.g. human, bowhead or blue whale, more preferably longer than human e.g. bowhead or blue whale;
(xxix) modified at its N- and/or C-terminal ends, optionally amidation/esterification of the C-terminus and/or acylation (e.g. acetylation) of the N-terminus;
(xxx) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) acylated to its N-terminus, non-limiting e.g. myristoyl/palmitoyl/stearoyl group conjugated to N-terminus;
(xxxi) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(xxxii) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C—C(COOH)—N—C(O)—(C)ₙ wherein n is between 2 and 100, preferably between 2 and 25, wherein (C) n can be linear or branched, saturated or unsaturated;
(xxxiii) part(s) or all is cyclized, in one or more cycles;
(xxxiv) part(s) or all is bicyclic via attachment to a scaffold(s), optionally rendered bicyclic by judicious insertion of cysteine residues which confer attachment to a scaffold structure by thioether and/or disulphide bonds;
(xxxv) Na-alkylated (e.g. N^{α}-methylated) at one or more places;
(xxxvi) contains one or more corresponding (to its sequence) D-amino acids;
(xxxvii) contains one or more retroinverse regions, or all of it is retroinverse;
(xxxviii) IF1 protein/fragment (or sequence variant thereof) component/entirety is retroinverse;
(xxxix) at least one of its carboxylic groups is esterified, optionally such that one or more of its carboxyl (COOH) groups is replaced by the group (or analogue thereof): 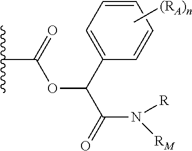
wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example:

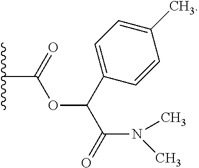 [SMILES: *C(=O)OC(C(=O)N(C)C)c1ccc(C)cc1]

6. IF1 protein/fragment (or sequence variant thereof), or fusion protein thereof, according to claim 5 wherein (from a sub-list(s) and or the overall list) x or more aspects/features/descriptors/modifications are true of it, wherein x is an integer selected from the group comprising: 2, 3, 4, 5, 6, 7, 8, 9, 10, 11, 12, 13, 14, 15, 16, 17, 18, 19, 20 [different values of x are different embodiments].

7. A fusion protein according to claim 6 comprising (or consisting of):
(i) at least one Mitochondrial Import Sequence (MIS) [for delivery into the mitochondrial matrix], optionally in the order (N-terminal shown first): [MIS]-[IF1 protein/fragment (or sequence variant thereof)]; or
(ii) at least one epitope/affinity tag and at least one MIS [for delivery into the mitochondrial matrix], optionally in the order (N-terminal shown first): [epitope/affinity tag]-[MIS]-[IF1 protein/fragment (or sequence variant thereof)]; or
(iii) at least one MIS [for delivery into the mitochondrial matrix] and at least one Cell Penetrating Peptide (CPP) sequence, optionally in the order (N-terminal shown first): [CPP]-[MIS]-[IF1 protein/fragment (or sequence variant thereof)]; or
(iv) at least one epitope/affinity tag and at least one MIS [for delivery into the mitochondrial matrix] and at least one CPP sequence, optionally in the order (N-terminal shown first): [epitope/affinity tag]-[CPP]-[MIS]-[IF1 protein/fragment (or sequence variant thereof)].

8. A fusion protein according to claim 7, wherein one or more of the following applies to (is true of) part(s) or all of it (wherein all possible combinations are contemplated {including all possible combinations of elements/descriptors within, and across, different bullet points} except those that are mutually exclusive):
(i) produced/isolated/purified/substantially purified/partially purified;
(ii) associated with a pharmaceutically/cosmetically acceptable salt[s];
(iii) Mitochondrial Import Sequence (MIS) is the same that a species uses for one or more of its proteins that it transports from the cytoplasm into the mitochondrial matrix, optionally wherein the MIS is the same that a species uses for its native IF1 protein;
(iv) MIS is that for an IF1 protein from human or mouse;
(v) MIS and IF1 protein/fragment (or sequence variant thereof) are from different species;
(vi) MIS and IF1 protein/fragment (or sequence variant thereof) are from different species, optionally wherein the former is from a species that will be administered with the fusion protein and the latter is from a different species, preferably from a longer-living species than the subject (to be administered) species, preferably from a very long-lived species (e.g. bowhead or blue whale);
(vii) MIS and IF1 protein/fragment (or sequence variant thereof) are from same species;
(viii) IF1 protein/fragment is from Bos taurus/human/bowhead whale/blue whale/mouse/rat/naked mole rat (or sequence variant thereof), MIS is from a different species;
(ix) MIS is from human, and IF1 protein/fragment (or sequence variant thereof) is from a different species, optionally Bos taurus/whale/bowhead whale/blue whale/mouse/rat/naked mole rat;
(x) MIS is from mouse, and IF1 protein/fragment (or sequence variant thereof) is from a different species, optionally Bos taurus/whale/bowhead whale/blue whale/rat/naked mole rat;
(xi) MIS is from a species, and IF1 protein/fragment (or sequence variant thereof) is from a longer-living species (higher maximal lifespan);
(xii) MIS is from a species, and IF1 protein fragment (or sequence variant thereof) is from a shorter-living species (lower maximal lifespan);
(xiii) MIS is from a species, and IF1 protein fragment (or sequence variant thereof) has more residues from the N-terminal than C-terminal half of IF1 protein, preferably from a longer-living species (higher maximal lifespan);
(xiv) MIS is from a species, and IF1 protein fragment (or sequence variant thereof) has more residues from the C-terminal than N-terminal half of IF1 protein, preferably from a shorter-living species (lower maximal lifespan);
(xv) MIS is from a species, and the IF1 protein fragment (or sequence variant thereof) is from a different species, preferably a longer-living species (higher maximal lifespan), and comprises (or consists of) {using “mature” [without MIS] IF1 protein numbering} one or more of (or sequence variant thereof) 10-47, 13-47, 14-47, 1-56, 1-57, 1-58, 1-59, 1-60, 10-56, 10-57, 10-58, 10-59, 10-60, 42-58, 42-59 residues;
(xvi) MIS is from a species, and the IF1 protein fragment (or sequence variant thereof) is from a different species, preferably a shorter-living species (lower maximal lifespan), wherein it is shorter than 40 (or 35, or 30, or 25, or 20, or 15, or 10) amino acids, and contains the H49 residue (using “mature” [without MIS] IF1 protein numbering);
(xvii) is (in total or in part[s]) retroinverse, optionally wherein the Mitochondrial Import Sequence (MIS) is not retroinverse;
(xviii) MIS is excluded from being retroinverse, but other part(s) can be;
(xix) MIS is not retroinverse but the IF1 protein/fragment (or sequence variant thereof) is retroinverse (in entirety or in part[s]);
(xx) MIS is not retroinverse but the IF1 protein/fragment (or sequence variant thereof) and/or Cell Penetrating Peptide (CPP) sequence is retroinverse (in entirety or in part[s]);
(xxi) CPP component(s) is one or more of a Tat sequence(s) (and/or sequence variant(s) thereof of the art), Penetratin sequence(s) (and/or sequence variant(s) thereof of the art), poly-Arginine sequence(s) (and/or sequence variant(s) thereof of the art),
optionally YGRKKRRQRRRG [SEQ ID NO:446] (optionally wherein the terminal glycine is absent), optionally RRRRRRRG [SEQ ID NO:461] (optionally wherein the terminal glycine is absent), optionally wherein one or more of the amino acids can be corresponding D-amino acids, optionally wherein part(s) or all of the CPP component(s) is retroinverse;
(xxii) epitope/affinity tag component(s) comprises (or consists of) one or more of poly-histidine, any of SEQ ID NO:130 to SEQ ID NO:144 e.g. HHHHHHDYDDDDK [SEQ ID NO:136];
(xxiii) CPP component(s) is flanked on one or both sides, optionally just on its C-terminal end (which is concatenated to the MIS component), by zero or more glycine and/or proline residues, optionally between 0 and 5 residues, optionally 1 such residue;
(xxiv) CPP component(s) is bound to the remainder of the fusion protein by a disulphide (by judicious insertion and/or substitution of cysteine residues) or peptide bond(s), or mixture thereof (i.e. some of the IF1 fusion proteins have their CPP bound by a disulphide bond, others by a peptide bond), optionally wherein a cysteine residue is at the C-terminal end of the CPP, which is disulphide bonded to an inserted/substituted N-terminal/internal cysteine in the MIS or IF1 protein/fragment (or sequence variant thereof) component of the fusion protein, optionally wherein the cysteine is (using “mature” [without MIS] IF1 protein numbering) present at (in legacy to the IF1 protein/fragment, or sequence variant thereof, used e.g. if from gray whale), or substituted into, the 37^{th} position;
(xxv) modified at its N- and/or C-terminal ends, optionally amidation/esterification of the C-terminus and/or acylation (e.g. acetylation) of the N-terminus;
(xxvi) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) conjugated/acylated to its N-terminus, non-limiting e.g. myristoyl/palmitoyl/stearoyl group acylated to N-terminus;
(xxvii) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(xxviii) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C-C(COOH)—N—C(O)—(C)ₙ wherein n is between 2 and 100, preferably between 2 and 25, wherein (C)ₙ can be linear or branched, saturated or unsaturated;
(xxix) part(s) or all is cyclic, optionally bicyclic, optionally where it is rendered bicyclic by judicious insertion of cysteine residues which confer attachment to a scaffold structure by thioether and/or disulphide bonds, optionally wherein, for a IF1 protein/fragment (or sequence variant thereof) containing fusion protein, if present, the CPP sequence(s) (optionally a CPP sequence(s) with precedent use in a bicyclic form from the literature) is confined to one cycle, and the MIS and IF1 protein/fragment (or sequence variant thereof) is confined to the other cycle of a bicyclic structure, optionally wherein the sequence attached to the scaffold is of the form (where IF1 below can refer to a “mature” IF1 protein(s) and/or a IF1 sequence variant(s)/fragment(s)/fragment sequence variant(s) thereof): Cys-CPP-Cys-MIS-IF1-Cys;
(xxx) N^{α}-alkylated (e.g. N^{α}-methylated) at one or more places;
(xxxi) comprises (or consists of) one or more corresponding (to its sequence) D-amino acids;
(xxxii) is (in total or in part[s]) retroinverse, optionally wherein if it contains a Mitochondrial Import Sequence (MIS), this is not retroinverse, optionally wherein the Mitochondrial Import Sequence (MIS) is not retroinverse and the IF1 protein/fragment (or sequence variant thereof) and/or Cell Penetrating Peptide (CPP) sequence is retroinverse (in entirety or in part[s]);
(xxxiii) at least one of its carboxylic groups is esterified, optionally such that one or more of its carboxyl (COOH) groups is replaced by the group (or analogue thereof): 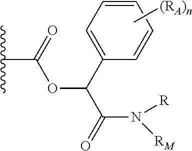
wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group,
for example:

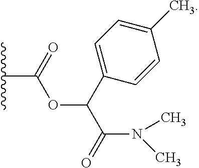 [SMILES: *C(=O)OC(C(=O)N(C)C)c1ccc(C)cc1]

9. A (optionally produced/isolated/purified/substantially purified/partially purified) peptide/protein comprising (or consisting of) at least one sequence selected from SEQ ID NO:166 to SEQ ID NO:438, or a fragment thereof (for non-limiting example wherein the epitope/affinity tag component [if present] is absent, and/or the Cell Penetrating Peptide component [if present] is absent), or concatenated fragments thereof, and/or a sequence variant(s) thereof (which is very preferably functional {can inhibit/reduce F₁F₀ ATP hydrolysis in a cell and/or in a Sub-Mitochondrial Particle [SMP] assay of F₁F₀ ATP hydrolysis}, optionally incorporating one or more conservative substitutions), optionally wherein one or more of the listed options in one or more of claims 5-8 apply to the sequence[s] (non-limiting e.g. associated with a pharmaceutically/cosmetically acceptable salt[s], esterified, modified at N- and/or C-terminal ends, N-terminal pre-sequence attached, cholesterol derivative(s) and/or fatty acid(s) [or derivative(s) thereof] attached, cyclized, bicyclic, corresponding D-amino acid at one or more places, one or more parts are retroinverse, N^{α}-alkylated {e.g. N^{α}-methylated} etc. [all combinations contemplated except those that are mutually exclusive]).

10. A (optionally produced/isolated/purified/substantially purified/partially purified) polynucleotide, optionally cDNA, encoding at least one peptide/protein sequence from one or more of claims 5-9,
optionally wherein one or more of the codons, optionally all the codons, used for each amino acid are the most (or one of the most) frequently used for each amino acid in the codon bias of at least one species that will express the polynucleotide;
and/or
wherein the polynucleotide comprises (or consists of) one or more sequences selected from SEQ ID NO:1426 to SEQ ID NO:1684,
or a pharmaceutical/cosmetic composition thereof.

11. A vector/plasmid of the art (non-limiting e.g. liposome, nanoparticle, lipid nanoparticle [LNP] etc.), or a pharmaceutical/cosmetic composition thereof, comprising at least one polynucleotide of claim 10; one or more vectors/plasmids (of the art) each comprising at least one polynucleotide of claim 10.

12. A cell comprising at least one vector/plasmid of claim 11; one or more cells each comprising at least one vector/plasmid of claim 11; optionally wherein the cell(s) can be bacterial (non-limiting e.g. E. coli), yeast (non-limiting e.g. Saccharomyces cerevisiae), immortalized mammalian (non-limiting e.g. human) cell line, insect cell, or other cell type(s) used for recombinant protein expression in the art.

13. A method/process for producing/manufacturing a protein(s)/peptide(s) from claims 5-9 comprising culturing (e.g. in/atop a nutrient medium) one or more cells of claim 12 under conditions suitable for the expression of at least one polynucleotide of claim 10, and recovering said protein(s)/peptide(s) therefrom, optionally by way of an epitope/affinity tag sequence component, optionally wherein this tag is then removed, optionally by the epitope/affinity tag sequence being connected to one end of the desired peptide/protein sequence(s), optionally the N-terminal end, by a cleavable linker sequence that is cleaved.

14. A gene therapy/vector of the art, or a pharmaceutical/cosmetic composition thereof, comprising at least one polynucleotide of claim 10, optionally a gene therapy/vector of the art for (disproportional) delivery to:
(i) one or more skin/scalp cells; and/or
(ii) one or both eyes/ears; and/or
(iii) one or more brain regions and/or one or more brain cell/neuron/glia types/populations; and/or
(iv) a population(s) of cells/body region(s), e.g. a population of brain cells/brain region(s)/eye cells, that tends to age faster, and/or lose optimal function earlier in life, than other parts of the body (in that species), optionally wherein this loss is a drive to an age-correlated disease(s)/disorder(s) e.g. a neurodegenerative disease (e.g. Parkinson's disease), e.g. an age-correlated eye disorder(s)/disease(s) such as Age-related Macular Degeneration (AMD);
optionally wherein the gene therapy vector is an Adeno-Associated Virus (AAV), optionally AAV9 or AAV2;
especially preferred is using a gene therapy vector that is used in an FDA/EMA approved gene therapy and/or that has passed Phase I clinical trialing or/and otherwise proven safe in humans.

15. A transgenic organism, preferably/restrictively a non-human transgenic organism, optionally a transgenic microorganism, optionally a non-human transgenic mammal, optionally a transgenic mouse, containing at least one polynucleotide of claim 10.

16. At least one peptide/protein sequence of claims 5-9, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of claim 10 [and/or at least one vector(s) of claim 11, and/or gene therap(y/ies) of claim 14, and/or at least one cell(s) of claim 12, and/or at least one transgenic organism(s) of claim 15] for use in the manufacture of a medicament or a pharmaceutical/cosmetic composition;
at least one peptide/protein sequence of claims 5-9, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of claim 10 [and/or at least one vector(s) of claim 11, and/or at least one gene therap(y/ies) of claim 14, and/or at least one cell(s) of claim 12, and/or at least one transgenic organism(s) of claim 15] for use in the manufacture of a medicament for the treatment/amelioration/prevention/combat/reversal/slowing/delaying of aging (and/or increasing lifespan and/or healthspan), and/or an unwanted/undesirable aspect(s)/sign(s) of aging and/or an age-correlated (risk of incidence increases with subject age) disorder(s)/disease(s) (e.g. a neurodegenerative disease[s]), in a subject;
at least one peptide/protein sequence of claims 5-9, and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide of claim 10 [and/or at least one vector(s) of claim 11, and/or at least one gene therap(y/ies) of claim 14, and/or at least one cell(s) of claim 12, and/or at least one transgenic organism(s) of claim 15] for use in the manufacture of a medicament/cosmetic/supplement for the treatment/amelioration/prevention/combat/reversal/slowing/delaying of skin/scalp aging and/or one or more signs of skin/scalp aging/photoaging/age-correlated damage (non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.), optionally in a pharmaceutical/cosmetic/supplement composition of the art for topical/skin/scalp/transdermal (e.g. human skin/scalp) administration, optionally a cream/lotion/spray/gel/oil/liquid/foam/paste/aerosol/butter/patch/make-up/shampoo/soap thereof.

17. Method of screening for least one IF1 protein fragment that can inhibit/reduce F₁F₀ ATP hydrolysis in a Sub-Mitochondrial Particle [SMP] assay of F₁F₀ ATP hydrolysis, at alkaline pH (e.g. pH 8), in which endogenous/native IF1 protein is not removed,
preferably wherein a number of different IF1 protein fragments are systematically tested, preferably by one or more of the following methods:
(1) wherein the first IF1 protein fragment tested consists of the most C-terminal (last) residue of an IF1 protein (non-limiting e.g. of Bos taurus), the second fragment tested consists of the last two residues, the third fragment consists of the last three residues, the fourth fragment consists of the last four residues, and this testing is iterated in this fashion, adding a residue each time (optionally testing until the N-terminal end of the IF1 protein is reached, or stopping before this, optionally stopping once the 47^{th} or 42^{nd} residue [from the N-terminal end, using “mature” {without MIS} IF1 protein numbering] is reached, or when a residue nearby is reached);
then, with each fragment found to have inhibition/reduction of F₁F₀ ATP hydrolysis activity, the fragment sequence is tested again but with its most C-terminal (last) absent, and then again iteratively, each time with one more amino acid removed from its C-terminal end, until the activity is lost or until there are no residues remaining;
(2) wherein the first IF1 protein fragment tested consists of IF1 protein residues (using “mature” [without MIS] IF1 protein numbering): 42-58, and then in the next test, the 43-58 fragment is tested, then 44-58, then 45-58 etc., wherein with each new test, the fragment has one less amino acid at its N-terminal end, until there are no more fragments left to test (the same method but coming instead from the C-terminal end is contemplated also);
optionally wherein one or more of the following apply (wherein all possible combinations are contemplated {including all possible combinations of elements/descriptors within, and across, different bullet points} except those that are mutually exclusive):
(i) the SMPs are from a mammal;
(ii) the SMPs are from a species that is to be administered with the IF1 protein fragment(s) (and/or sequence variant(s) thereof), or fusion protein(s) thereof, selected by the assay, and/or from a closely/not too distantly related species (e.g. if humans are to be administered, the SMPs can be from bovine);
(iii) the assayed IF1 protein fragment(s) (and/or sequence variant(s) thereof) is/are from a mammal;
(iv) the IF1 protein fragment(s) (and/or sequence variant(s) thereof) assayed is/are from a species that is to be administered with the IF1 fragment(s), or fusion protein(s) thereof, selected by the assay, and/or from a closely/not too distantly related species;
(v) the IF1 protein fragment(s) (and/or sequence variant(s) thereof) is from a short(er)-living species, but preferably a species not too evolutionarily divergent from a species that is to be administered with the IF1 fragment(s) (and/or sequence variant(s) thereof), or fusion protein(s) thereof, selected by the assay, wherein shorter living mammals have more strongly/tightly joined IF1 tetramers (and higher oligomers), wherein they are bound to each other in their C-terminal halves, and so a C-terminal IF1 fragment from a shorter-living mammal binds a C-terminal part of an IF1 protein more tightly (if the evolutionary distance between them isn't too great);
optionally wherein the method(s) is repeated with fragments from the IF1 protein of a different species, optionally wherein it is performed with fragments from the IF1 protein of a number of different species;
optionally wherein each IF1 protein fragment(s) (or sequence variant(s) thereof) selected by this method (i.e. shown to reduce F₁F₀ ATP hydrolysis) is tested in an SMP assay of F₁F₀ ATP synthesis, optionally wherein if it appreciably reduces F₁F₀ ATP synthesis also, it is dismissed.

18. A vector of the art for a polynucleotide, preferably a gene therapy vector of the art, optionally an Adeno-Associated Virus (AAV) [optionally AAV2 administered to the eye(s)], comprising at least one polynucleotide coding for at least one [any] IF1 protein/fragment
(or sequence variant thereof,
optionally wherein {using “mature” [without MIS] IF1 protein numbering} it has a H49K (or H49A or H49R) substitution and, if its 14^{th} residue is not alanine, it is substituted to be alanine),
optionally derived from that of a long lived mammal species (e.g. whale species [longer living species preferred e.g. bowhead or blue whale]) and/or long-lived reptile species e.g. a tortoise/turtle/terrapin etc.)
or a pharmaceutical/cosmetic composition thereof;
especially preferred is using a gene therapy vector that is used in an FDA/EMA approved gene therapy and/or that has passed Phase I clinical trialing or/and otherwise proven safe in humans.

19. At least one (optionally produced/isolated/purified/substantially purified/partially purified) [any] IF1 protein/fragment (or sequence variant thereof), and/or a fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or a fusion protein(s) thereof (optionally containing a CPP sequence(s)) [and/or vector(s)/plasmid(s)/liposome/(s)/nanoparticle(s)/gene therap[y/ies] thereof, and/or cell(s)/transgenic cell(s)/transgenic organism(s) thereof], for use in the manufacture of a medicament or a pharmaceutical/cosmetic composition;
at least one (optionally produced/isolated/purified/substantially purified/partially purified) [any] IF1 protein/fragment (or sequence variant thereof), and/or fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or fusion protein(s) thereof (optionally containing a CPP sequence(s)) [and/or vector(s)/plasmid(s)/liposome(s)/nanoparticle(s)/gene therap[y/ies] thereof, and/or cell(s)/transgenic cell(s)/transgenic organism(s) thereof], for use in the manufacture of a medicament for the treatment/amelioration/prevention/combat/reversal/slowing/delaying of aging (and/or increasing lifespan and/or healthspan), and/or an unwanted/undesirable aspect(s)/sign(s) of aging and/or an age-correlated (risk of incidence increases with subject age) disorder(s)/disease(s) (e.g. a neurodegenerative disease[s]), in a subject;
at least one (optionally produced/isolated/purified/substantially purified/partially purified) [any] IF1 protein/fragment (or sequence variant thereof), and/or a fusion protein(s) thereof (optionally containing a CPP sequence(s)), and/or at least one pharmaceutically/cosmetically acceptable salt, solvate, hydrate, prodrug, liposome, nanoparticle (e.g. lipid nanoparticle, LNP) or other vector of the art thereof, and/or at least one polynucleotide coding for at least one [any] IF1 protein/fragment (or sequence variant thereof) and/or a fusion protein(s) thereof (optionally containing a CPP sequence(s)) [and/or vector(s)/plasmid(s)/liposome(s)/nanoparticle(s)/gene therap[y/ies] thereof, and/or cell(s)/transgenic cell(s)/transgenic organism(s) thereof], for use in the manufacture of a medicament/cosmetic/supplement for the treatment/amelioration/prevention/combat/reversal/slowing/delaying of skin/scalp aging and/or one or more signs of skin/scalp aging/photoaging/age-correlated damage (non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.), optionally in a pharmaceutical/cosmetic/supplement composition of the art for skin/scalp/transdermal (e.g. human skin/scalp) administration, optionally a cream/lotion/spray/gel/oil/liquid/foam/paste/aerosol/butter/patch/make-up/shampoo/soap thereof,
optionally wherein one or more of the following apply to it (wherein all possible combinations are contemplated {including all possible combinations of elements/descriptors within, and across, different bullet points} except those that are mutually exclusive):
(i) comprises at least one [any] IF1 protein (or sequence variant thereof) and at least one [any] IF1 protein fragment (or sequence variant thereof);
(ii) comprises multiple different IF1 protein and/or IF1 protein fragment sequences, optionally wherein one or more are sequence variants, optionally wherein two or more are different (optionally overlapping) fragments from the same IF1 protein (from the same species) or from the same sequence variant thereof;
(iii) at least one IF1 protein(s)/fragment(s) (or sequence variant thereof) is from a long-lived species (e.g. with a long maximal lifespan) e.g. from a long lived mammal species (e.g. whale species [longer living species preferred e.g. bowhead or blue whale]) and/or long-lived reptile species e.g. a tortoise/turtle/terrapin etc;
(iv) at least one IF1 protein(s)/fragment(s) (or sequence variant thereof) is from human.

20. At least one compound and/or composition for use in a method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises increasing the amount of (at least one type of) IF1 protein (and/or sequence variant(s) thereof) in the subject.

21. At least one compound and/or composition for use according to claim 20 wherein at least some of the extra IF1 protein is an IF1 protein sequence from a different species, optionally a longer-living (higher maximal lifespan) species than the subject's species, optionally a very long-lived species (high maximal lifespan).

22. At least one compound and/or composition for use according to claim 20, wherein at least some of the extra IF1 protein is an IF1 protein (from same or different species than the subject, optionally from a longer-living species, optionally from a very long-lived species) sequence variant with (using mature {without MIS} IF1 protein residue numbering) lysine or alanine or arginine as its 49^{th} residue, and/or alanine as its 14^{th} residue.

23. At least one compound and/or composition for use according to claim 20, wherein a majority, optionally all, of the subject's cells have an increased amount of (at least one type of) IF1 protein (and/or sequence variant(s) thereof).

24. At least one compound and/or composition for use in a method of treating/ameliorating/preventing/combating/reversing/slowing/delaying aging in a subject (and/or, in the subject, increasing their lifespan and/or healthspan and/or treating/ameliorating/preventing/combating/reversing/slowing/delaying an unwanted/undesirable aspect(s)/sign(s) of aging and/or one or more of an age-correlated disorder(s)/disease(s)/damage(s)/sign(s)/decline in function(s)/decline in aesthetic(s)) wherein the method comprises administering to the subject (and/or the subject self-administering), systemically and/or locally/topically to the subject's body part(s)/organ(s)/tissue(s)/cell population(s)/cell(s) where the effect(s) is (most) sought (e.g. to one or more areas of skin/scalp e.g. one or more areas of the face, e.g. to one or both eyes/ears, e.g. to one or more joints), optionally as part of a pharmaceutical/cosmetic/supplement composition/medicament, an amount (preferably an effective amount e.g. a therapeutically/cosmetically effective amount) of (optionally produced/isolated/purified/substantially purified/partially purified) at least one [any] IF1 protein/fragment (or sequence variant thereof) and/or (optionally produced/isolated/purified/substantially purified/partially purified) at least one fusion protein comprising at least one [any] IF1 protein/fragment (or sequence variant thereof), and/or at least one pharmaceutically/cosmetically acceptable salt, solvate, hydrate, prodrug, liposome, nanoparticle (e.g. lipid nanoparticle, LNP) or other vector of the art thereof, optionally comprising an N-terminal Cell Penetrating Peptide (CPP) sequence(s) concatenated to a Mitochondrial Import Sequence(s) (MIS(s)) concatenated to a (preferably “mature” i.e. without its MIS) IF1 protein/fragment (or sequence variant thereof), and/or (optionally produced/isolated/purified/substantially purified/partially purified) at least one polynucleotide coding for at least one [any] IF1 protein/fragment (or sequence variant thereof), and/or a fusion protein(s) thereof, and/or a vector(s)/plasmid(s)/liposome(s)/nanoparticle(s)/gene therap[y/ies]/cell(s) thereof, and/or at least one pharmaceutical/cosmetic composition thereof.

25. At least one compound and/or composition for use according to claim 24 wherein one or more of the following applies/is true (wherein all possible combinations are contemplated {including all possible combinations of elements/descriptors within, and across, different bullet points} except those that are mutually exclusive):
(i) at least one IF1 protein/fragment (or sequence variant thereof), and/or at least one IF1 protein/fragment (or sequence variant thereof) containing fusion protein, is according to one or more of claims 5-9;
(ii) at least one polynucleotide is according to claim 10;
(iii) at least one vector/plasmid is according to claim 11;
(iv) at least one cell is according to claim 12;
(v) at least one gene therapy is according to one or more of claims 14, 18;
(vi) at least one medicament or pharmaceutical/cosmetic composition is according to one or more of claims 16, 19;
(vii) at least one IF1 protein/fragment (or sequence variant thereof), and/or IF1 protein/fragment (or sequence variant thereof) component to a fusion protein, is part, or the entirety, of the subject's species' native IF1 protein, or a sequence variant thereof;
(viii) at least one IF1 protein/fragment (or sequence variant thereof), and/or IF1 protein/fragment (or sequence variant thereof) component to a fusion protein, is from a species that tends to live longer (e.g. has a greater maximal lifespan) than the subject's species, optionally/preferably from one of the most long-lived species on Earth e.g. bowhead whale;
(ix) at least one IF1 protein/fragment (or sequence variant thereof), and/or IF1 protein/fragment (or sequence variant thereof) component to a fusion protein, is a fusion of fragment(s)/residue(s) of IF1 protein sequences from two or more different species, optionally with one or more (preferably single residue) substitutions/insertions/deletions atop at one or more positions, preferably at less than 10 positions;
(x) at least one IF1 protein/fragment (or sequence variant thereof), and/or IF1 protein/fragment (or sequence variant thereof) component to a fusion protein, has a Mitochondrial Import Sequence [MIS] attached (preferably by peptide bond to its N-terminal end) that is the same as an MIS that the subject's/subject's species' uses to transport one or more of its endogenous/native proteins from the cytoplasm to the mitochondrial matrix, optionally the MIS that the subject's/subject's species' uses for its endogenous/native IF1 protein;
(xi) at least one IF1 protein fragment (or sequence variant thereof) is administered;
(xii) at least one IF1 protein (or sequence variant thereof) and at least one IF1 protein fragment (or sequence variant thereof) is administered;
(xiii) multiple different IF1 protein and/or IF1 protein fragment sequences, optionally wherein one or more are sequence variants, optionally wherein two or more different (optionally overlapping) fragments from the same IF1 protein (from the same species) or from the same sequence variant thereof, are administered;
(xiv) at least one IF1 protein/fragment (or sequence variant thereof) is functional as a stand-alone/separate peptide/protein i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it confers greater inhibition of F₁F₀ ATP hydrolysis than a naturally occurring IF1 protein at pH 8;
(xv) at least one IF1 protein/fragment (or sequence variant thereof) has one or more conservative substitutions, and/or one or more non-conservative substitutions, and is functional i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater inhibitory activity against F₁F₀ ATP hydrolysis than a naturally occurring IF1 protein at pH 8;
(xvi) at least one IF1 protein/fragment (or sequence variant thereof) has notable/high sequence identity (e.g. one or more of ≥28%, ≥30%, >40%, ≥50%, ≥60%, ≥70%, ≥75%, ≥80%, ≥85%, ≥90%, ≥95%, ≥96%, >97%, ≥98%, ≥99% sequence identity and/or less than 12, or less than 10, or less than 8, or less than 6, or less than 5, or less than 4, or less than 3, or 1 to 2 single residue substitutions/insertions/deletions away from a native IF1 protein sequence or fragment(s) thereof) to the entirety, or part, of at least one IF1 protein from a species, and is functional i.e. it can inhibit/reduce F₁F₀ ATP hydrolysis (e.g. inside a cell, preferably a eukaryote cell, and/or in a Sub-Mitochondrial Particle (SMP) assay of the art, which assays F₁F₀ ATP hydrolysis [functional in an SMP assay with native IF1 protein removed and/or present]), optionally/preferably wherein it has greater inhibitory activity against F₁F₀ ATP hydrolysis than a naturally occurring IF1 protein at pH 8.

26. At least one compound and/or composition for use according to one or more of claims 20-25 wherein administration is local/topical, instead of systemic, and so any ensuing reduction in endogenic/metabolic heat production in (and optionally around) the administered area (caused by less F₁F₀ ATP hydrolysis in that area) is compensated for by heat transfer from other body regions, especially via blood flow, maintaining (at or close to) the optimal body temperature (e.g. ˜37° C. in a mammal) in/around the administered area;
optionally wherein administration is local/topical to the skin/scalp, optionally in a pharmaceutical/cosmetic/supplement composition of the art for skin/scalp/transdermal (e.g. human skin/scalp) administration, optionally a cream/lotion/spray/gel/oil/liquid/foam/paste/aerosol/butter/patch/make-up/shampoo/soap thereof, optionally wherein this administration acts to slow/delay/reverse/treat/ameliorate/prevent/combat skin/scalp aging (e.g. one or more signs of skin aging/photoaging/age-correlated damage: non-limiting e.g. lateral canthal lines (crow's feet), liver/age spot(s), wrinkle(s) [e.g. facial wrinkles], fine lines in the skin (e.g. around eyes and/or mouth), expression lines, dark circles/“bags” under the eyes, hair greying/loss etc.);
optionally wherein administration is local/topical to one or both eyes, optionally in a pharmaceutical composition of the art for eye administration, optionally one or more of eye drop(s), intravitreal injection(s), contact lens coating/solution (optionally wherein the contact lens has little to no refractive ability or wherein the contact lens is prescriptive to the refractive defect/error of the subject's eye(s)) thereof, optionally wherein this administration acts to slow/delay/reverse/treat/ameliorate/prevent/combat eye(s) aging and/or at least one eye(s) aging related disease/disorder, including any eye disease(s)/disorder(s) whose likelihood of onset increases with age and/or worsens with age, including (to illustrate and not restrict) age-related macular degeneration (AMD, early/intermediate/late), age-related wet macular degeneration, neovascular/wet AMD, dry AMD, Geographic atrophy (G A), wet and dry AMD in the same eye(s), Stargardt's macular degeneration, Best vitelliform macular dystrophy, diabetic retinopathy, proliferative diabetic retinopathy, diabetic macular edema, vision loss, progressive vision impairment, myopia (short-sightedness), degenerative myopia, hyperopia (far-sightedness), accommodative dysfunction, glaucoma, progressive glaucoma, cataract formation, retinal degeneration, progressive retinal degeneration, retinitis pigmentosa, leber hereditary optic neuropathy, Fuchs spot, Best's disease, Sorsby's fundus dystrophy.

27. At least one compound and/or composition for use according to one or more of claims 20-25 wherein administration is systemic, and optionally the subject is monitored, for example by a healthcare professional(s) and/or machine substitute(s), for sign(s) of reduction in body temperature and/or the subject is located at an ambient temperature that maintains their body temperature within safe limits whilst they have an effective amount of administered compound(s)/composition(s) in their system and/or the subject wears (and/or is covered by) insulating material(s), e.g. clothing/clothes (and/or bedding/blanket(s)), and/or is in a heated/insulated space and/or hot climate, optionally exceeding 25° C. or 28° C. or 30° C. or 35° C. or 36° C. or 37° C., optionally at or around 37° C., wherein a high (e.g. in the thirties ° C. i.e. 3x° C. where x is a number between 0 and 9), but safe, ambient temperature (and/or greater bodily insulation, for example by clothing/clothes and/or bedding/blanket(s)) can permit a greater compound(s)/composition(s) dose(s) to be safely administered, wherein a preferred ambient temperature is the thermoneutral temperature for the subject with the amount of bodily insulation they have, e.g. the amount of clothing they are wearing, if any, and the amount of the administered compound(s)/composition(s) in their body/system;
optionally wherein the subject wears one or more items of clothing, and/or is sheltered, and/or is in a heated and/or insulated confinement/room/space, some or all of the time whilst they have an amount (e.g. effective amount e.g. a therapeutically/cosmetically effective amount) of the administered compound(s)/composition(s) in their body/system;
optionally wherein the subject is administered (and/or self-administers) the compound(s)/composition(s) shortly before they sleep, preferably wherein they are sheltered (e.g. inside instead of outside) and/or insulated (e.g. by bedding(s)/blanket(s), and/or clothing, and the like) whilst they sleep, optionally in a heated room/building/confinement that is set to a higher (safe) temperature than outside it.

28. An IF1 protein/fragment (or sequence variant thereof), or fusion protein thereof, optionally a pharmaceutically/cosmetically acceptable salt, solvate, hydrate, prodrug, liposome, nanoparticle (e.g. lipid nanoparticle, LNP) or other vector of the art thereof, wherein one or more of the following applies to it (wherein all possible combinations are contemplated {including all possible combinations of elements/descriptors within, and across, different bullet points} except those that are mutually exclusive), wherein some functional significance is disclosed in this present claim:
(i) contains one or more corresponding (to its sequence) D-amino acids;
(ii) contains one or more retroinverse regions, or all of it is retroinverse, optionally wherein the Cell Penetrating Peptide (CPP) component (if present) is retroinverse (in part or entirety) and/or the IF1 protein/fragment (or sequence variant thereof) is retroinverse (in part or entirety);
(iii) N^{α}-alkylated (e.g. N^{α}-methylated) at one or more places;
(iv) part(s) or all is cyclized, in one or more cycles;
(v) part(s) or all is bicyclic via attachment to a scaffold(s), optionally rendered bicyclic by judicious insertion of cysteine residues which confer attachment to a scaffold structure by thioether and/or disulphide bonds;
(vi) modified at its N- and/or C-terminal ends, optionally amidation/esterification of the C-terminus and/or acylation (e.g. acetylation) of the N-terminus;
wherein one or more of the aforementioned features reduces susceptibility to protease(s) in the blood and increases peptide/protein half-life in the blood circulation of a subject (increases its plasma stability);
(vii) at least one of its carboxylic groups is esterified, optionally such that one or more of its carboxyl (COOH) groups is replaced by the group (or analogue thereof): 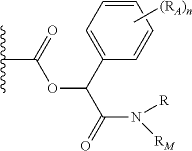
wherein R_{A} is (independently at each point of use) an alkyl or alkoxy (optionally at the para position on the indicated phenyl ring) or halogen, n is between 0 and 3, R is an alkyl, alkenyl, alkynyl group or hydrogen, R_{M} is an alkyl, alkenyl, alkynyl, cycloalkyl, aryl, or arylalkyl group which is optionally substituted with one or more alkyl, alkoxy, aryl, alkylaryl, halogen, haloalkyl, or haloalkoxy group, for example: 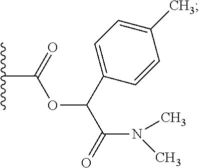 [SMILES: *C(=O)OC(C(=O)N(C)C)c1ccc(C)cc1]
wherein this esterification confers (or enhances) the ability to passage a biological/plasma membrane(s), wherein the moiety/moieties attached by an ester bond are cleaved off by esterases once the peptide/protein enters a cell;
this esterification also (sterically) reduces susceptibility to proteases in the blood and so increase plasma half-life;
(viii) at least one IF1 protein/fragment (or sequence variant thereof), and/or IF1 protein/fragment (or sequence variant thereof) containing fusion protein, has a fatty acid (optionally linear or branched, saturated or unsaturated, containing 2 to 100 carbon atoms, more preferably from 2 to 25 carbon atoms; or derivative thereof) acylated to its N-terminus, non-limiting e.g. myristoyl/palmitoyl/stearoyl group conjugated to N-terminus;
(ix) a sequence, containing a number (integer selected from between 1 and 8) of amino acid residues (optionally wherein one or more are hydrophobic {optionally at least as hydrophobic as phenylalanine}, and/or one or more are positively charged {e.g. are lysine and/or arginine}, and/or one or more are reasonably hydrophobic with an ability to adopt a positive charge {e.g. histidine}), is peptide-bonded to the N-terminus, optionally wherein a fatty acid (or a derivative thereof) is conjugated/acylated to the resultant N-terminus of this fusion protein, and/or conjugated/acylated to the side-chain of one or more of these additional amino acid residues, optionally wherein at least one of these additional residues is a lysine, and a fatty acid is conjugated/acylated to its side-chain (optionally via a “spacer” moiety, which can be for non-limiting example, an amino acid(s) [e.g. L-γ-glutamic acid] or a dipeptide(s), or L-γ-glutamic acid and two OEG {8-amino-3,6-dioxaoctanoic acid} units), optionally wherein at least one of these added residues is cysteine, and a cholesterol derivative (e.g. cholesterol modified with a cysteine-reactive 2-bromoacetyl moiety) or fatty acid derivative (e.g. [hydrogen atoms not shown]: S—C—C(COOH)—N—C(O)—(C)ₙ wherein n is between 2 and 100, preferably between 2 and 25) is conjugated to its side-chain by a disulphide bond, preferably wherein this added sequence only has one lipophilic (fatty acid/cholesterol or derivative thereof) moiety attached in total, wherein a preferred fatty acid is linear or branched, saturated or unsaturated, containing between 2 to 100 carbon atoms, more preferably between 2 to 25 carbon atoms, optionally wherein an attached fatty acid is a myristoyl/palmitoyl/stearoyl group;
(x) at least one cysteine residue in the amino acid sequence, optionally inserted/substituted into the sequence, optionally a cysteine present or substituted into the correspondent position to the (using “mature” [without MIS] IF1 protein numbering) 37^{th} amino acid position of the IF1 protein/fragment (or sequence variant thereof) [incidentally, gray whale IF1 protein has a cysteine residue in this position], which has a cholesterol derivative (e.g. cholesterol modified with a cysteine-reactive 2-bromoacetyl moiety) or a fatty acid (or derivative thereof) conjugated to it, preferably via a disulphide bond, optionally wherein the fatty acid derivative is [hydrogen atoms not shown]: S—C—C(COOH)—N—C(O)—(C)ₙ wherein n is between 2 and 100, preferably between 2 and 25, wherein (C)ₙ can be linear or branched, saturated or unsaturated;
conjugated fatty acid (or derivative thereof) confers self-association and/or binding to albumin in the blood, which (sterically) decreases protease(s) access to peptide/protein, and/or slows its renal clearance, thence increasing its half-life in blood (e.g. from minutes to hours);
conjugated cholesterol/fatty acid (or derivative thereof) increases lipophibicity and confers (or enhances) the ability to passage a biological/plasma membrane(s);
when cholesterol/fatty acid (or derivative thereof) is attached by a disulphide bond, this attachment breaks once inside the reducing intracellular environment;
the pre-sequence/residue increases peptide/protein lipophibicity, which confers (or enhances) its ability to passage a biological/plasma membrane(s), and/or contributes positive charge which enhances the ability to passage into a live cell (negative inside);
in preferred cases, wherein this pre-sequence/residue/attachment is more N-terminal in the fusion protein than a Mitochondrial Import Sequence (MIS), it is inherently cleaved from the IF1 protein/fragment (or sequence variant thereof) when the MIS is cleaved off, in the mitochondrial matrix;
(xi) Cell Penetrating Peptide (CPP) component(s) to fusion protein, which comprises (or consists of) R7 [SEQ ID NO:455] or RRRRRRRG [SEQ ID NO:461] or RRRRRRRP [residues 4-11 of SEQ ID NO:453];
confers better cellular penetration than a Tat sequence; correspondent to amino acid sequences found within the human and mouse proteome and so less immunogenic in those species (amongst others) than a Tat sequence (for example); optional terminal glycine (G) or proline (P) confers flexibility at its C-terminal connection point to fusion protein;
(xii) Mitochondrial Import Sequence (MIS) and IF1 protein/fragment (or sequence variant thereof) components to fusion protein are from different species;
this permits the MIS to be from a species that will be administered the fusion protein, facilitating its better delivery into the mitochondrial matrix in that species, and the IF1 protein/fragment (or sequence variant thereof) to be from a different, longer living species (a species with a greater maximal lifespan);
(xiii) IF1 protein/fragment (or sequence variant thereof) component/entirety comprises (or consists of) that from a long-lived species, preferably at least as long-living as Bos taurus, more preferably from one of the longest-lived species/mammals on Earth e.g. bowhead or blue whale;
(xiv) one or more (or two/three/four/five/six/seven/eight/nine/ten/eleven/twelve/thirteen/fourteen/fifteen/sixteen/seventeen or more) of the following is true of the IF1 protein/fragment (or sequence variant thereof) component/entirety: (using “mature” [without Mitochondrial Import Sequence (MIS)] IF1 protein numbering): 49^{th} residue is not histidine, 14^{th} residue is not a residue that can be phosphorylated (i.e. is not serine or threonine), 26^{th} residue is not glutamic acid, 48^{th} residue is not histidine, 55^{th} residue is not histidine, 56^{th} residue is not histidine, 49^{th} residue is lysine or alanine or arginine, 14^{th} residue is alanine, 26^{th} residue is alanine or glutamine, 48^{th} residue is alanine, 55^{th} residue is alanine, 56^{th} residue is alanine, 79^{th} residue is glycine or asparagine, 76^{th} residue is lysine, 73^{rd} residue is serine, 62^{nd} residue is histidine, 82^{nd} residue is aspartic acid, 83^{rd} residue is aspartic acid, 84^{th} residue is aspartic acid, 85^{th} residue is aspartic acid, 57^{th} residue is valine, 54^{th} residue is serine or aspartic acid, 61^{st} residue is glutamine, 51^{st} residue is asparagine, 47^{th} residue is glutamic acid, 46^{th} residue is arginine, 44^{th} residue is serine, 39^{th} residue is lysine, 38^{th} residue is alanine or glutamic acid, 37^{th} residue is arginine or cysteine or lysine, 36^{th} residue is aspartic acid or glutamic acid, 29^{th} residue is histidine, 27^{th} residue is alanine, 25^{th} residue is lysine, 17^{th} residue is aspartic acid, 12^{th} residue is glycine, 11^{th} residue is serine or threonine, 10^{th} residue is serine or glycine, 9^{th} residue is glycine, 8^{th} residue is leucine or glycine, 6^{th} residue is aspartic acid or glycine, 5^{th} residue is alanine, 4^{th} residue is serine or glycine, 3^{rd} residue is glutamic acid or serine or lysine, 2^{nd} residue is glycine, 1^{st} residue is leucine;
(xv) IF1 protein/fragment (or sequence variant thereof) component/entirety is truncated, without C-terminal regions required for dimerization, teteramerization and higher oligomerization, for example it is only IF1 protein residues {using “mature” [without MIS] IF1 protein numbering}: 14-47 (or 10-47 or 13-47 or 1-56 or 1-58 or 1-60 or 10-56 or 10-58 or 10-60), preferably wherein if its 14^{th} residue isn't alanine, it is substituted to be alanine;
(xvi) IF1 protein/fragment (or sequence variant thereof) component/entirety is truncated, without the ability to inhibit F₁F₀ ATP hydrolysis itself, but wherein it binds at least one part in the C-terminal half of a complete IF1 protein, wherein (because it is truncated appropriately) its binding doesn't occlude the more N-terminal IF1 protein component that inhibits F₁F₀ ATP hydrolysis, but it does occlude one or more of the IF1 protein parts involved in tetramer (and higher) oligomerization (sequesteration) at alkaline pH (e.g. pH 8, normal pH of mitochondrial matrix), liberating IF1 dimers/monomers that can inhibit F₁F₀ ATP hydrolysis at pH 8; advantageously such an IF1 protein/fragment (or sequence variant thereof) tends to be shorter (better for intracellular delivery) than a IF1 protein/fragment (or sequence variant thereof) that can inhibit F₁F₀ ATP hydrolysis itself (e.g. aforementioned IF1 protein residues: 14-47); this particular approach relies on the endogenous IF1 protein and so works best in longer-living species which tend to have more, and/or more potent, IF1 protein; with this approach it is preferred for the IF1 protein/fragment (or sequence variant thereof) to be from the species to be administered or a shorter-living species (IF1 protein from a longer-living species binds ATP synthase more tightly/potently, and other IF1 proteins {to form IF1 protein tetramers and higher oligomers} less tightly; IF1 protein from a shorter-living species binds ATP synthase less tightly/potently and other IF1 proteins {to form IF1 protein tetramers and higher oligomers} more tightly; so shorter-living species have more tightly bound IF1 tetramers {and higher oligomers} and so a IF1 protein/fragment {or sequence variant thereof} solely designed to bind another IF1 protein is better sourced from a shorter than a longer-living species, but preferably from a species not too far evolutionarily removed from a/each species to be administered); preferably this IF1 protein/fragment (or sequence variant thereof) is less than 25 amino acids long, more preferably less than 20;
wherein one or more of the aforementioned features confers increased ability to inhibit/reduce F₁F₀ ATP hydrolysis at alkaline pH e.g. at pH 8 [which is the normal pH of the mitochondrial matrix] e.g. in a Sub-Mitochondrial Particle (SMP) assay of F₁F₀ ATP hydrolysis and/or in a cell and/or in a subject;
wherein one or more of the aforementioned features, when the peptide/protein is administered to a subject, confers increased ability to slow aging (conferring increased associated benefit(s), e.g. increased cosmetic(s) and/or therapeutic effect(s), thereof) in the subject.

29. An organism, preferably/restrictively a non-human organism, optionally a mouse, which has an H49K (or H49A or H49R), and/or S14A (or T14A), substitution in the IF1 protein produced by its mutated/modified ATPIF1 (ATP5IF1) gene,
or/and
a transgenic organism, preferably/restrictively a non-human transgenic organism, optionally a transgenic microorganism, preferably a non-human transgenic mammal, optionally a transgenic mouse, containing/expressing/constitutively expressing at least one transgenic ATPIF1 gene(s) (and/or sequence variant thereof; and/or IF1 protein/fragment [and/or sequence variant thereof] coding polynucleotide sequence[s] without (or with less) introns), optionally at least one (wherein all possible combinations are contemplated {including all possible combinations of elements/descriptors within, and across, different bullet points} except those that are mutually exclusive):
(i) polynucleotide sequence coding for/expressing at least one IF1 protein from a longer living species (longer maximal lifespan), preferably which (using “mature” [without MIS] IF1 protein numbering) has a H49K (or H49A or H49R) substitution and, if its 14^{th} residue is not already alanine, it is substituted to be alanine; or
(ii) polynucleotide sequence coding for/expressing an IF1 protein from blue whale (Balaenoptera musculus), preferably which (using “mature” [without MIS] IF1 protein numbering) has H49K (or H49A or H49R) and/or T14A substitutions; or
(iii) polynucleotide sequence coding for/expressing an IF1 protein from bowhead whale (Balaena mysticetus), preferably which (using “mature” [without MIS] IF1 protein numbering) has an H49K (or H49A or H49R) substitution; or
(iv) polynucleotide sequence coding for/expressing an IF1 protein from human, which (using “mature” [without MIS] IF1 protein numbering) has H49K (or H49A or H49R) and S14A substitutions; or
(v) polynucleotide sequence coding for/expressing at least one IF1 protein fragment (or sequence variant thereof) concatenated at its N-terminal end to a Mitochondrial Import Sequence (MIS, for transport into the mitochondrial matrix), preferably an MIS that the organism species uses for one of one or more proteins it transports from the cytoplasm to the mitochondrial matrix, more preferably the MIS that it uses for its endogenous/native IF1 protein, wherein the IF1 protein fragment (or sequence variant thereof) can optionally be:
(a) IF1 protein fragment (or sequence variant thereof) less than z amino acids long, wherein z is an integer selected from a group comprising 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 [different values of z are different embodiments]; or
(b) IF1 protein fragment (or sequence variant thereof) of (using “mature” {without MIS} IF1 protein numbering) x-y, where x is an integer between 1 and 20 (or between 1 and 44, or between 1 and 84), and y is an integer between 40 and 85 (or between 40 and 85, or between 2 and 85) [different values of x and/or y are different embodiments; within their aforementioned range constraints, all possible combinations of x and y integer values are contemplated]; or
(c) IF1 protein fragment (or sequence variant thereof) selected from a group comprising (using “mature” [without MIS] IF1 protein numbering): 1-84, 2-84, 3-84, 4-84, 5-84, 6-84, 7-84, 8-84, 9-84, 10-84, 11-84, 12-84, 13-84, 14-84, 15-84, 16-84, 17-84, 18-84, 19-84, 20-84, 21-84, 22-84, 23-84, 24-84, 25-84, 26-84, 27-84, 28-84, 29-84, 30-84, 31-84, 32-84, 33-84, 34-84, 35-84, 36-84, 37-84, 38-84, 39-84, 40-84, 41-84, 42-84, 43-84, 44-84, 45-84, 46-84, 47-84, 48-84, 49-84, 50-84, 51-84, 52-84, 53-84, 54-84, 55-84, 56-84, 57-84, 58-84, 59-84, 60-84, 61-84, 62-84, 63-84, 64-84, 65-84, 66-84, 67-84, 68-84, 69-84, 70-84, 71-84, 72-84, 73-84, 74-84, 75-84, 76-84, 77-84, 78-84, 79-84, 80-84, 81-84, 82-84, 83-84, O R a sub-sequence/fragment of one of these aforementioned fragments; or
(d) IF1 protein fragment (or sequence variant thereof) optionally selected from (using “mature” {without MIS} IF1 protein numbering): residues: 14-47, 13-47, 12-47, 11-47, 10-47, 9-47, 8-47, 7-47, 6-47, 5-47, 4-47, 3-47, 2-47, 1-47, 14-48, 14-46, 14-45, 14-44, 14-43, 14-42, 13-48, 13-46, 13-45, 13-44, 13-43, 13-42, 12-48, 12-46, 12-45, 12-44, 12-43, 12-42, 11-48, 11-46, 11-45, 11-44, 11-43, 11-42, 10-48, 10-46, 10-45, 10-44, 10-43, 10-42, 42-58, 42-59, 1-56, 1-57, 1-58, 1-59, 1-60, 10-56, 10-57, 10-58, 10-59, 10-60, 14-60, 10-84, 14-84, 18-84, 10-50, 1-45, 42-56, 42-47, 48-56, 49-55 (or sequence variant thereof of any fragment aforementioned); or
(e) IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60 (or sequence variant thereof); or
(f) bowhead whale IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60; or
(g) blue whale IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60, preferably with a T14A substitution; or
(h) human IF1 protein fragment (using “mature” {without MIS} IF1 protein numbering): residues: 10-47 or 13-47 or 42-58 or 42-59 or 42-56 or 1-56 or 1-58 or 1-60 or 10-56 or 10-58 or 10-60, preferably with a S14A substitution;
in one or more of:
(i) one or both its eyes;
(ii) forebrain/intestine/liver and at least one other brain region/body region/organ/tissue/cell population;
(iii) forebrain/intestine/liver and at least two other brain/body regions/organs/tissues/cell populations;
(iv) two or more of forebrain, midbrain and hindbrain;
(v) cell type(s)/cell population(s)/tissue(s)/organ region(s)/organ(s) that tends to age faster, and underperform/fail/lose optimal function earlier (optionally wherein this underperformance/failure/optimal function loss can cause a pathology/disease in a subject [e.g. of aging] and/or sign(s) of aging/older age/old age e.g. a neurodegenerative disease), than most cell type(s)/cell population(s)/tissue(s)/organ region(s)/organ(s) in the body;
(vi) in one or more dopamine neurons, preferably the majority/all, in the pars compacta (in the substantia nigra);
(vii) in more than one different cell population/tissue/organ region/organ/brain region (and/or one or more sub-parts/regions thereof), optionally in at least 15% or 25% or 50% or 75% or 90% or many/majority of the organism's cells/cell populations/tissues/organs, optionally in all;
preferably wherein this modified organism has a longer healthspan and/or lifespan than is typical for its species, optionally wherein, if the modified organism is a mouse, it has a lifespan in excess of 6 and/or 5 years, optionally wherein this organism is entered into one or more lifespan and/or healthspan assays (and/or competitions e.g. M Prize or similar) of the art, optionally wherein this longer healthspan and/or lifespan is only observed, especially if the organism is of a homeothermic species, when the following is adhered to:
the organism lives (is reared/housed/kept) at a higher/sufficiently high safe ambient temperature (e.g. at, or at a safe temperature in excess of, 25/30/37° C.), and/or is afforded more bodily insulation, to account for it having less endogenous/metabolic heat production and a higher thermoneutral/thermo-comfortable temperature.

30. At least one compound and/or composition for use in a method of any of claims 20-27 and/or a pharmaceutical/cosmetic/supplement composition/medicament/peptide/protein/vector/gene therapy of any of claims 16, 19, 28, wherein the unwanted/undesirable aspect(s)/sign(s) of aging and/or disorder(s)/disease(s) of aging (e.g. incidence increases with increased age/senescence) includes (to illustrate and not restrict) geriatric aging, age-associated decline, age-related/correlated disease/disorder/condition, aging frailty, frailty, frailty syndrome, wasting, sarcopenia, muscle weakness, weakness, muscle fatigue, weight loss, cachexia, functional decline, osteoporosis, sclerosis, kyphosis, reduction in bone density, cognitive decline, neurological decline, cognitive deficit, cognitive impairment, mild cognitive impairment, depression, degenerative diseases, neurodegenerative diseases, motor-associated neurodegenerative diseases, motor neuron disease, motor neuron dysfunction, amyotrophic lateral sclerosis (ALS), primary lateral sclerosis, progressive muscular atrophy, age-related muscular atrophy, age-related fat loss, progressive bulbar palsy, progressive supranuclear palsy, pseudobulbar palsy, hereditary spastic paraplegia, Parkinson's disease, parkinsonism, Multiple System Atrophy (MSA), Progressive Supranuclear Palsy (PSP), essential tremor, resting tremor, Alzheimer's disease, Huntington's disease, spinocerebellar ataxias, Friedreich's ataxia, cerebellar ataxia, dysautonomia, dementia, frontotemporal dementia, chronic traumatic encephalopathy, memory loss, aged cognition, age/aging related cognitive decline/impairment, congential epilepsy, Batten disease, polyglutamine diseases, atherosclerosis, atherosclerotic plaque in a blood vessel, arteriosclerosis, vascular stiffening, arterial stiffness, stiffened arteries, hypertension, cardiovascular disease(s), myocardial infarction, acute myocardial infarction, angina, arrhythmia, cardiomyopathy, congestive heart failure, coronary artery disease, carotid artery disease, endocarditis, coronary thrombosis, myocardial infarction, ischemia reperfusion injury, anemia, hypertension, aortic aneurysm, cardiac diastolic dysfunction, irregularity in heart rhythm, decrease in cardiac stress tolerance, increase in the cross-sectional area of cardiomyocyte(s), hypercholesterolemia, hyperlipidemia, mitral valve prolapsed, peripheral vascular disease, cardiac stress resistance, brain aneurysm, inflammatory or autoimmune disease, cerebrovascular disease, stroke, heart failure, heart failure with preserved ejection fraction, fibrosis, idiopathic pulmonary fibrosis (IPF), pulmonary fibrosis, fibrotic disease, cardiac fibrosis, liver fibrosis, pancreatic fibrosis, oral submucosa fibrosis, cystic fibrosis, gum recession, gingival recession, oral mucositis, pulmonary disease, age-related loss of pulmonary function, chronic obstructive pulmonary disease, emphysema, bronchiectasis, coronary artery disease, hypercholesterolemia, liver disease, fatty liver disease, lysosomal storage disease, amyloidosis, systemic sclerosis, kidney disease, chronic kidney disease, renal disease, renal failure, end-stage renal disease (ESRD), renal insufficiency, glomerulosclerosis, cirrhosis, hepatic cirrhosis, hepatic insufficiency, immunosenscence, clonal hematopoiesis, Chronic Obstructive Pulmonary Disease (COPD), emphysema, breathlessness, asthma, hypertension, hypercholesterolemia, age-related thymic atrophy, chronic inflammatory disease(s), joint pain, arthritis, osteoarthritis, osteoarthritis of knee(s), arthritis (Osteo- and Rheumatoid), Juvenile Rheumatoid Arthritis (JRA), arthrosis, herniated intervertebral disc, kyphosis, degenerative disc disease, vertebral disc degeneration, tendinopathy, androgenetic alopecia, male-pattern baldness, hair loss, Idiopathic Pulmonary Fibrosis, systemic sclerosis, Psoriasis, age-related loss of cardiac/pulmonary/cognitive/vision function, decrease in cardiac stress tolerance, insulin sensitivity, poor glycemic control, diabetes, type 1 diabetes, type 2 diabetes, diabetic ulcer, diabetic retinopathy, diabetic neuropathy, diabetic nephropathy (diabetic kidney disease), diabetic ulcer, boutonneuse fever, obesity, metabolic disease/syndrome/dysfunction, inflammatory bowel disease, andropause, glaucoma, progressive glaucoma, retinal degeneration, sarcopenia, cachexia, age-related cachexia and/or sarcopenia, macular degeneration, Age-related Macular Degeneration (AMD, early/intermediate/late), age-related wet macular degeneration, neovascular/wet AMD, dry age-related macular degeneration, dry AMD, Geographic atrophy (G A), dry age-related macular degeneration with geographic atrophy, wet and dry AMD in the same eye(s), Stargardt's macular degeneration, Best vitelliform macular dystrophy, retinopathy, diabetic retinopathy, proliferative diabetic retinopathy, diabetic macular edema, age/aging-related eye disease, ophthalmological/ophthalmic disease/disorder/condition, ocular disease, vision loss, blindness, progressive vision impairment, myopia (short-sightedness), degenerative myopia, hyperopia (far-sightedness), accommodative dysfunction, cataract formation, cataract(s), retinal degeneration, progressive retinal degeneration, presbyopia, vision loss, retinitis pigmentosa, leber hereditary optic neuropathy, Fuchs spot, Best's disease, Sorsby's fundus dystrophy, vaso-obliteration in eye(s), oxygen induced vaso-obliteration, neovascularization in eye(s), hearing loss (e.g. age-related), deafness, presbycusis, tinnitus, naive T cell shortage, movement disability, nonalcoholic fatty liver disease (NAFLD), nonalcoholic steatohepatitis (NASH), immunosenescence, immune senescence, poor immune response to a vaccine(s) (so countering this improves vaccine response=improves the protection conferred by a vaccine), respiratory/urinary tract infection (RTI/UTI) especially in older/aged/elderly subjects, loss of bladder control, lower urinary tract symptoms (LUTS), Benign Prostatic Hyperplasia (BPH), hyperplasia, polycystic kidney disease, cancer, age-related cellular hypertrophy, dermatological disease/disorder, eczema, psoriasis, hyperpigmentation, nevi, rashes, atopic dermatitis, urticaria, diseases/disorders related to photosensitivity/photoaging, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, a hallmark(s) of aging, genomic instability, telomere attrition, epigenetic alteration(s), loss of proteostasis, deregulated nutrient sensing, mitochondrial dysfunction, cellular senescence, stem cell exhaustion, altered intercellular communication, homeostatic imbalance, reduced fitness, reduced reproductive fitness, infertility, female infertility, menopause, incontinence, sleep disturbances, imbalance, fear, depression, ulcers,
wherein the unwanted/undesirable aspect(s)/sign(s) of aging and/or disorder(s)/disease(s) of aging includes (to illustrate and not restrict) accelerated/premature aging, any accelerated/premature aging disease, any progeroid syndrome, including (to illustrate and not restrict) premature aging because of chemo-/radio-/cancer therapy, Werner syndrome, Bloom syndrome, De Barsy syndrome, Rothmund-Thomson syndrome, Cockayne syndrome, xeroderma pigmentosum, trichothiodystrophy, combined xeroderma pigmentosum-Cockayne syndrome, restrictive dermopathy, Wiedemann-Rautenstrauch syndrome, Hutchinson-Gilford progeria syndrome (progeria), a laminopathy, Ataxia telangiectasia-like disorder 2, XFE progeroid syndrome, Muscular dystrophy, Muscular Dystrophy (Becker's, Duchenne, Limb-Girdle), Yamamoto's Muscular Dystrophy, Mandibuloacral dysplasia, Dilated cardiomyopathy, GAPO syndrome, Cutis laxia, Ehlers-Danlos syndrome, Lenz-Majewski hyperostatic dwarfism, SHORT syndrome, Progressive external opthalmoplegia, Nester-Guillermo progeria syndrome, MDPL syndrome, Dyskeratosis congenital, Down syndrome.
